SEQUENCE LISTING (758 sequences)

SEQ ID NO: 1 - protein, 1424 aa, unidentified
  source 1..1424: note = MG36-5 effector
MKRILGLDLGTNSIGWALVNEAETDEEVSSIIRLGVRVNPLTVDETQNFEKGKSITTNAERTLKRSMRRNLQRYKLRRDALVEFLKELGFITDETILSEQGNRTTFETCRLRAKAAEEEISLEELSRVLLMINKKRGYKSSRKVKKEEDGVLIDGMEIAKKLYEEDLTPGQLCSQLLESGKKVLPDFYRSDLQEELDRIWSVQKQFHPDVFCDAAKEEIKGKNRSQTWAILANYFVWKEEVVGWNDREAKNETIEKECKLVGLKRTTKGYELKKENYRWRAQALTEQLGLEEIAVVLQEINGQINASSGYLGAISDRSKVLYFNHQTVGQYQMAELDKNPHASLRNMVFYRQDYLDEFDRIWEKQAEFHKELTAELKKEIRDMIIFYQRRLKSQKGLISFCEFERLEIVVEKDGKKQTKVIGCKVIPRSHPLFQEFKVWQTLNDIKVLGREKKRNALSASESRALYPEEKEILARELAIKENMKKADVLKLLFENPQELDLNFKQIDGNRTGFSLFSAYSKMIEKYGYEPLDFKKPADEIIGQLRTIFTDLGWNTNLFTIDLTKEGKELEMQPYFRLWHLLYSFEGDNTSTGNGKLIEKIMQLCGVEKEYAVELASLSFQDDYGSLSAKAIKRILPYLKEGNLYDVACEYAGYRHSKSSLTKEEIENKVLKGKLEVLPKNSLRNPVVEKILSQMVNVINTIIDTYGKPDEIRVELARELKKSAKEREELTKAIAKSTREHEEIRKLLQDEFGMMNVSRNDIIRYKLYEELKDNGYKTLYSNQYIPKEKIFSKEIDIEHIIPQSRLFDDSLSNKTLEYKAINIEKGNKTAYDFVKEKYGEEGLQQYINRCESLFNDKKAKLRKLKMEQKDIPDGFIDRDLRNTQYIAKKALAMLNEICRRVVATTGSITDQLREDWQLVDVMKELNLPKYEALGFVETYEDKDGRKIKRIKDWTKRNDHRHHAMDALTVAFTKDAFIQYFNNKNAAQDSGNRNHTNIIGIKTRYFDKGRALPPIPLEQFRIEAKQHLEKLLVSIKAKNKVVTVNVNRTKKRKGENTKIQQTPRGQLHLETVYGSHKQYVTKIEKVNASFDAAKIATVSKRAYRNALLKRLETFGNDPKKAFTGKNALEKNPLYADKYQTIRVPEKVQTVEFETIYTIRKPVDPALNVDKVVDVKVRAILERRLKEYGGDPKKAFVNFWENPIWLNKEKGISIKRVSIRGINNAQSIHVKKDKNGNPIWDENGKQIPVDFVNTGNNHHVAIYRKPVLDKKGQVTFDEDGNLMYELDEVVVPFFEAVTRANLGLPIIDKDYRKSEGWQFLFSMKQNEYFVFPNEKTGFNPKEVDLLNPDNYAMISPNLFRVQTMSKVMYGNNVVRDYKFRHHLETTVKDMKELKDIAYKQYKTLSFGNSVVKIRINHIGQIVSVGEY

SEQ ID NO: 2 - protein, 264 aa, unidentified
  source 1..264: note = MG36-5-B1 transposition protein
MKADVSERQRVKLLADMIGYYRLCCPRIGGFKLFHLLEKDLGHAVTLGRDSFLKVYESKGFKLNPNKRRRTTDSNHVYKRYPNLIKGKDARYSNHIWVSDITYVWILGDVLYLHLVTDAYSHAVLGWCLSDSLSASHTTEALRMAIRIAGGGNLCGTIHHSDRGSQYASEAYVSCLMEHHIRISMTEGYEPTDNAMAERQNGIFKVEWIYEQEMYRDKEQAINEINRMIDFYNNRRPHMSIGMECPMEVYKGKLPGKNLWRKRP

SEQ ID NO: 3 - protein, 126 aa, unidentified
  source 1..126: note = MG36-5-B2 transposition protein
MIKRKPKRVFTEPFKLSVLREYYSSGMSKYAITKKYSLSPPCIYRWLKEYPVGSDQLPLPSETKERLQMVPKQSDLTDMESLQKRIEELRRSLELEKMRSRAFEKMIEIAEEEEGISIFRKDGAKQ

SEQ ID NO: 4 - protein, 286 aa, unidentified
  source 1..286: note = MG36-5-T1 transposition protein
MARPNKEGLDYFPFDVDFFSDEKIGSISGEFGIKGEITAIKLLCAIYRNGYFILWNDALKMSLLRGLPGISLELLEQIVTRLVRWGFFEQTLFSTVSVLTSKGIQERYFKAIKRRKDSSNYPYLLVNVDNNKVNVSNNDINVNTNPIKERKGNKNRESLNTRETLFDNFKNELLRDEEWRRYACQISGLSVAFNDLIPGELDNFLAWMVSTGEGDTLKTIDDVKRRFTYWWQGTGLRAYNQRYGGTRKETFGGYTSHAGAYGKREAPAKTGVQPSEEARKDYTERF

SEQ ID NO: 5 - protein, 203 aa, unidentified
  source 1..203: note = MG36-5-C transposition protein
MIKALGCNYLGIERRQFETDRGNDKVLRFLLYYFNDCPLAESVFPEENYKLHKNLLIVGDPGTGKTLMMQIFADYLKLTDNPKRFVNLSVTQMMNYYKIHGHIDRFTYNEEAGKGSMEGNPFDICLNDIGLETENQKSYGTSLNSVIDEFLYARYEIYQSHQKKYHITSNLSVTDFKNRFGTRLVDRFKSFNVIILNGESRRR

SEQ ID NO: 6 - protein, 724 aa, unidentified
  source 1..724: note = MG39-1 effector
VKIIKRSGAENTFDKEKIENAVAKANITVEEKDRLSEGEIGEIAQNIEDKCSEMNRAMDVETIQDWVEADIMRHGKYTVAKHYITYRYERSIVRQANTTDKQILSLLNFENEEVKQENSNKNPTVNSVQRDYMAGEVSKDITRRFLLPDDIVEAHEKGLIHFHDADYFAQHMHNCCLVNLEDMLQNGTVISEVMIEKPHSFSTACNIATQSIAQIASSQYGGQSITLSHLAPFVQISRDKYRREVKKEFAELNIPADEDTINKVAEMRVKAEIVQGVQMIQYQVITLMTTNGQAPFVTVFMYLDEVPEGQTRDDLAAIIEEMLRQRIQGVKNEKGVYITPAFPKLIYVLEEDNIREGSKYWELTKLAAKCTAKRMVPDYISEKKMKELKVDKNGNGQCYPCMGCRSFLTTYLDENGKPKYYGRFNQGVVTINLVDVACSSYKDMDKFWLIFDERLELCRRALMLRHERLKGTPSDVAPILWQNGALARLKKGETIDKLLFGGYSTISLGYAGLCECVRYMTGKSHTDPSATPFALEVMQHLNDACAKWRAETNIDFSLYGTPLESTTYKFARCLQKRFGVIEGVTDRNYITNSYHIHVTENIDAFDKLTFESQFQALSPGGAISYVEVPNMQNNIEAVLAVMQHIYDNIMYAELNTKSDYCQKCGFDGEIKIVEDDGKLVWECPNCGNRDQNTLNVARRTCGYIGTQFWNQGRTQEIKERVLHL

SEQ ID NO: 7 - protein, 214 aa, unidentified
  source 1..214: note = MG39-1-A transposition protein
MSKRISRATKLARGQGSGEGAKYKSYVMTNEFNSLGTTSVIKDWKTGRGVHCLSQAEALWFYILRWDDNNIDIREQYPLDRNITSRIADKYGFKHPGNSDHIMTTDFLVTKKNNKLHAYSVKPDRDLSKRTLEILCIEKLYWEHNNIEFDMLFKEDVNTILASNIRLVTEYYDESRVFDRYSNIRHNIATKKIRCDMEHKILTNDDLDRIWGEY

SEQ ID NO: 8 - protein, 716 aa, unidentified
  source 1..716: note = MG39-1-B transposition protein
MTILIEYGVNTEMNDYVLAIGDILYDKDNQNSYRIISLIDDHLILCEMETTKLELQQIKYTIIADLVLSNKIEIKKDQALVYDIDQLSESVRNRYILKVHIMNDVKIAYGPSYLGLMGKSSKIELQKILAKYNYPISSFWRMCTTYFQSGMKNYSLINAKSFKSNEVKTYTYKARPGAKSTYNLDNVVKPNEYVIYFEEALNEYKAGREKTLKNAFSRMNILHFTQAEIIDGVATRLLLPECQRPTYKQFYYYAQKHLTKEEKDLIKTSAAEQRNNKRLLISDSLKDVYGPADMVEIDACEADVSLVSELDPDQAIGRPIVYFMIDVYSRIILAVSVAFDNNSILGITNLFLNLADDKQEYCKKYGIEFNDKRLWPSGVIPKRIRVDRGSEFKSYEFDRICNELGIEKQIVSGASGSLKGVVEQAFHQMHAKQNVHLENHGLIEKRYDSLHHKEASLTIHDYTRMVINFVLAHNQQHLETYPLTKEMIEKNIAPVPAILWEYGSKKYGMPQPIPVLEQYLFSLMTPIKAKISKRGISYKGLWYFAPNDKRLMSEMYAAGTRRMPFEVRMDMRDVGAIYYIRNSKLVKIPLNVLITGNSDYKGLTMKQYEEYYSAKKKMQAKGRIDNEKIDTAVYANNESIVKSAKKNVHSRTKDIRSSREIDKQKVSYEGKISARLENKDETSKNISSDEQKENGVTEYRDYASFEEALQDFYDNN

SEQ ID NO: 9 - protein, 534 aa, unidentified
  source 1..534: note = MG39-1-C transposition protein
MAKYNYVTCEEVVSQYDDYDLKEQRIVPARYVEAKLAIDTGNPYIEALPYPRTGRNIISSYSQTMADFDYDKIKSMSTIDKILQIRSLRSIRFPLPFHAELELSFYNALITSYRSRHILHSDNDKVSYSVENQEYAASNILVGDSSASTDAGFSLIGYSGCGKSSAIQMLVSYYPQVIMHTTENGEYFPQITYLVVNCIPNSNFSALYDGIGDAIDKALGNIKPIYSAEIMKIRTLGAKAERIREYVEKFAIGIIIFDEIQLIDFSHTRENSFDSLLTLSNRTKVATAIVGTEDAKAKMFKTLRTARRVGNVINGNMYCMDKKFFYFLVNELFRYQWFDKPVTVSEEITDALYDVTKGIVDQLIGIYIYMNIDYLNKKKKPEINSKYIYAVTNKHFAGLRDVLENLDSDFNNELMQEINDNAKLELDKIEDEAKQLKEAEKILNNNSSEEKVQIKNVVANITAIYDEYTPTQIEDAFKKVIRKKSSVGKAEREISKLVIEQLTKQQRTRSDLCKKNTPSVIQMQNFLGIDKEDK

SEQ ID NO: 10 - protein, 641 aa, unidentified
  source 1..641: note = MG39-1-Q transposition protein
MPVIPVYINPYPDELMYSWIHRLAKKNGLPITTFADSYLNKFNSKIGSLEYDIRYGLLCLNESFFIQKDLKEMFLSMSIFPFEAMFFSVGQQTRYVNNMFRKPDPLNAPVNTMIREIHICPQCIENDIEIFGEPYIHRAHQLSGVCTCHKHKAPLYKYTGIKGHECEYDLSHYTELEIKDIAIENEYTDYAQALFNSNTNCNIADLKHMIYNKLKELGHKATNGYEDFISAFNDSKLVTLFDADLKNFLLVSIISTQSTSARSMLPLLMYLFPNVQEIIHRFENAPPIIQKYHCAECGKNFYATPTSLTEGWGCTYCDANKSIDERYKKLIDFAGKGNYEPLEPFRSLNIKSKIHHKICGETIQIKPRKFIFDHVRCICESLLNEWDVRQKLEKFEDYEFISYDSESSKITMRSKACGHVFSCRFHKFIKYPSCRVCRPKNMTTELYTERVYDLVGDDYTVLSEFVDQRTKIAIKHNKCGGIQEYKPSAFLDGQRCNACNSLIVKKANDSWEKGYALLCEYKEECGTANIPKRDHYKGVFLGNWLQSQRDKYKAGKLTRSQEDALVSLGITLDPLAAEWERRYEQYKRYIQQNNGSSDITKRTIFEGEKLGVWVVLQRRNYNIGKLSEERYKKLCDINMKF

SEQ ID NO: 11 - RNA, 118 nt, synthetic construct
  source 1..118: mol_type = other RNA; note = MG36-5 effector tested sgRNA sequence
nnnnnnnnnnnnnnnnnnnnnnnngttgtgatttgctttcagaaaatgaagcaaatcacaataaggattattccgttgtgaaaacattaggttccctcgcccttctgcgggggatttt

SEQ ID NO: 12 - RNA, 80 nt, synthetic construct
  source 1..80: mol_type = other RNA; note = MG36-5 effector tracrRNA sequence
tactaaaatgaagcaaatcacaataaggattattccgttgtgaaaacattaggttccctcgcccttctgcgggggatttt

SEQ ID NO: 13 - RNA, 46 nt, synthetic construct
  source 1..46: mol_type = other RNA; note = MG39-1 effector tracrRNA sequence 1
tttttgtaaaatcattttgtaaaattacaattcataatgagatatg

SEQ ID NO: 14 - RNA, 66 nt, synthetic construct
  source 1..66: mol_type = other RNA; note = MG39-1 effector tracrRNA sequence 2
tttttgtaaaatcattttgtaaaattacaattcataatgagatatgacaattcataatgagatatt

SEQ ID NO: 15 - RNA, 109 nt, synthetic construct
  source 1..109: mol_type = other RNA; note = MG39-1 effector tracrRNA sequence 3
aaaatataggatattaatgtcagagatatctcattataattgtaattttgttttgtgtacaagctgttaatatctcattatgaattgtcatatctcattatgaattgta

SEQ ID NO: 16 - RNA, 88 nt, synthetic construct
  source 1..88: mol_type = other RNA; note = MG39-1 effector tracrRNA sequence 4
aaaatataggatattaatgtcagagatatctcattataattgtaattttgttttgtgtacaagctgttaatatctcattatgaattgt

SEQ ID NO: 17 - DNA, 36 bp, synthetic construct
  source 1..36: mol_type = other DNA; note = putative MG36-5 transposon end LE1
ttaattgaaaattgaaaattgagaattgaaaattaa

SEQ ID NO: 18 - DNA, 36 bp, synthetic construct
  source 1..36: mol_type = other DNA; note = putative MG36-5 transposon end LE2
tgagaattgaaaattgagaattgggaattgagaatt

SEQ ID NO: 19 - DNA, 28 bp, synthetic construct
  source 1..28: mol_type = other DNA; note = putative MG36-5 transposon end RE1
ttaattttcaattctcaattttcaatta

SEQ ID NO: 20 - DNA, 36 bp, synthetic construct
  source 1..36: mol_type = other DNA; note = putative MG39-1 transposon end LE1
aaaagtattgacaaattgacaaaatggtggtataat

SEQ ID NO: 21 - DNA, 36 bp, synthetic construct
  source 1..36: mol_type = other DNA; note = putative MG39-1 transposon end RE1
attataacctatatttttgattttgtcaatagtttt

SEQ ID NO: 22 - protein, 637 aa, unidentified
  source 1..637: note = MG64-2 effector
MSQITIQCQLVASASTRQQLWLLMTQKNTPLINELLQQVGQHPEFETWRQKGKLQAGIVKALCQPLKADPRFMGQPARFYASAIAVVDYIYRSWLALQKRLQYQLEGQTRWYQMLRSDAELIELYGGSLETLRSKAAEILAQFAPESASVNPQPTKGKKSKKRKNSSNNPNLSTALFEAYRQTEDILSSCAINYLLKNGCKVSEKEEDPEKFAKRRRSVEIRIERLKEKLASRMPKGRDLTDEKWLETLMVASTTVPSSEFQAKSWQDNLLRKSSAIPFPVAYETNEDMTWFKNSKGRICVKFNGLSEQTFEVYCDSRQLYWFQRFLEDQQIKRNSKDQHSSSLFTLRSGRIAWSEGEGKGDPWNIHRLTLYCCVDTRLWTSEGTEQVRHEKADEITQIITKTKQKTDLNEKQQAFIKRKTSTLARINNPFPRPSKPVYQGHSHILVGVSLGLDKPATLAVIDAIANKVITYRSIRQLLGDNYQLLNRQRQQQHQNAHKRQTAQRQDAPHQFSESELGQYIDRLLAKAIVAVAKTYQAGSIVLPQLGDVRESIESEIKARAEQKCPDLVEVQKQYAKQYRSSIHRWSYARLIDSIKSQASQVGIFIEEGKQPVRGSPQDKAKDLAIAVYHSRLNTKS

SEQ ID NO: 23 - protein, 560 aa, unidentified
  source 1..560: note = MG64-2-B transposition protein
MQDDRESEVPIPAEVNEIVTDFSEDAKLMQEVIQSLLEPCDRITYGQRQREAAAKLGKSVRTIRRLVKKWETEGLSALQPNKRTDKGKHRIDQEWQEFIIKTYKEGNKGSKRITPQQVAVRVAAKAADLGQEKYPSYRTVYRVLQPIIEKQEKTQSVRSRGWRGSRLSVKTRDGQDLSVEYSNHVWQCDHTRADILLVDQDGQLLGRPWLTTVIDTYSRCIIGINLGYDAPSAYVVALALRHAILPKQYSSEYKLHCQWGTYGKPEHFYTDGGKDFRSNHLQQIGVQLGFVCHLRDRPSEGGIVERPFGTFNTDLFSTLPGYTGSNVQERPEEAEKEASLTLRELEQLLVRYIVDKYNQSIDARMGDQTRFQRWEAGLIAAPNLISERDLDICLMKQTRRTIYRGGYLQFENLTYRGDYLEGYSGESVVLRYDPKDITTILVYRKEGDKEVFLARAYEQDLQTEQLSFDEVKAISRKLREAKKAVNSRSILAEVRDRETFLTQKKSKKQRQKSEQAQIHRKKELFPIEAEATEFESPVNELETEIIEVFDYEQMREDYGF

SEQ ID NO: 24 - protein, 287 aa, unidentified
  source 1..287: note = MG64-2-C transposition protein
MMNQEKEAKAIAQKLGNIPLNDEKIQAEIQRLNRKNFVPLEQVKALHDWLESKRQARQCCRVIGESRTGKTMACNAYRLRNKAIQSLGQPPTVPVVYIQIPQECTPKELFSVLLEHLNHQMTKGTTAEMRNRTLRVLKACRVEMLIIDEADRLKPKTFADVRDIFDNLEISVVLVGTVRLEKVMTDDEQVCNRFSACYRYGKLSVEEFKKTVNIWENQVLKLPVSSNLTQSKMLDILKDKTQCYIGLMDMILRDAAIRALNKGMPKIDLETLKEVTNEYTAPPKRSK

SEQ ID NO: 25 - protein, 168 aa, unidentified
  source 1..168: note = MG64-2-Q transposition protein
MKASDIQPWLFRVEPFEGESISHFLGRFQRANELTPSGLAKAAGLGGAIARWEKFRFNPPPSPQQLEALALVVEVEAERLVQMLPPAGVGMKMEPIRLCGACYAEVPCHKIEWQFKTTQGCDRHQLSLLSECPNCGARYKVPALWPDGWCSRCFLPFADMVKWQKLIV

SEQ ID NO: 26 - protein, 637 aa, unidentified
  source 1..637: note = MG64-4 effector
MSQITIQCRLVCCESTRASLWKLMAEKNTPLTNELLHQVGHHCEFESWRKKGKIPSGIVKELCQPLKTDPRFTGQPGRFYTSAIALVSYIYKSWLVLMKRSHYKLEGKTRWLEMLSSDAELVERCGISVDGIRARASEILAQLALPDSADTGQVKGRKAKKSKKFQQQQDRDRNLSRTLFEAYRDTEDILTRCAICYLLKNGCQVSDREENSEKFTQRRRQVEIQIERLKEKLAARIPKGRDLTDAKWLETLFLATSHAPESEAEAKLWQDCLLRQPSSVPFPVAYETSEDMTWFKNHKGRISVKFNGLSEHTFEVYCDSRQLHWFERFLEDQQIKRNNQNQYSSSLFTLRSSRIAWQEGKGKGEPWNFNHLTLYCSVDTRLWTAEGTQLVIEEKVEEIVKNITNTKDKGNLNEKQLAHIQRKNSTLARITNPFPRPSKSIYRGQSHILVGVSLGLEKPATVAVVDATTSNVIAYRSIKQLLGDNYRLLNRQRQQKQVLSHQRHLAQRLDALNSLGESELGQYVDRLLAKEIVAIAQTYCAGSIVLPSLGDMREQVQSEIQAKAEQKSDLIEVQQKYAKQYRASVHQWSYGRLISSIQSQAKKAGIAIEEAKQPIRGSPQQKAKELVIAAYNARKTS

SEQ ID NO: 27 - protein, 563 aa, unidentified
  source 1..563: note = MG64-4-B transposition protein
MSKLSEDNHIDNQKPAVGEIVPEIADDNKQLLEVIQKLLEPCDRITYGQRQREAAAQLGKSVRTVRRLVKKWEEEGLAALSQTTRADKGKHRIEQDWQDFIIKTYKEGNKGSKRITPKQVAVRVQAKAAELGQDRYPSYRTVYRVLQPIIERQEQQASVRSRGWRGSRLSVKTRDGKDLSVEYSNHVWQCDHTRVDVLLVDRNGAILSRPWLTTVVDTYSRCIMGFNLGYDAPSSQVVALALRHAILPKQYDWEYQLDCDWGTYGKPEHFYTDGGKDFRSNHLQQIGVQLGFASHLRDRPSEGGIVERPFGTFNTEFFSTLPGYTGSNVQERPEQAEKEARLTLRELERLLVRYIVDKYNQSIDARLGDQTRFQRWEAGLIAAPNPIAERDLDICLMKQTRRSIYRGGYLQFENLTYRGENLAGYAGESVVLRYDPRDITTVLVYRQEAGKEVFLARAFAQDLETEQMSLDEAKASSRKLRETGKTISNRSILAEVRDRETFLTQKKTKKERQKAEQAEVKRAKQPFSVEREEEIEAASIPNQPEPEMPDVFDYEQMREDYGF

SEQ ID NO: 28 - protein, 278 aa, unidentified
  source 1..278: note = MG64-4-C transposition protein
MSLKDAQAIAQQLGNIQPNDARLQTEIQRLNGKSFVPLEQVKILHDWLDGKRQARQGCRVVGESRTGKTIACDAYRLRHKPIQELGKPPIVPVAYILVPPDCGSKDLFGLIVEHLKYQMTKGTVAEIRERTRRVLKGCGVEMLIIDEADRLKPNTFRDVRDIGENLGITVVLVGTDRLDAVIKKDPQVYNRFRACHRFGNLSGDDFKRTVEIWEKKVLQLPVASNLSGKTMLKTLGEATGGYIGLLDMILREAAIRALKKGLQKIDLETLKEVAGEYR

SEQ ID NO: 29 - protein, 167 aa, unidentified
  source 1..167: note = MG64-4-Q transposition protein
MEASDIQPWLFRAEPYEGESISHYLGRFRRANDLTPTGLGKATGLGGAIARWEKFRFNPPPSRKQLEALAVVVGVEADRLAQMIPPAGVGMKLEPIRLCAACYAQAPYHRIEWQFKVTAGCDHHQLRLLSECPNCKARFKIPALWVDGWCQRCFTKFEEMTSNQAAL

SEQ ID NO: 30 - protein, 632 aa, unidentified
  source 1..632: note = MG64-6 effector
MSQITIQCRLIAKESSRQALWRLMAELNTPLINDILNQIANHPDFETWREKGKLPAGIVKQLSDSLKTDPRYIGQPGRFYTSAITLISYIYKSWFKVQQRLQQRLVGQTRWLGILKSDEELVAESDRTLEEIRAKAIGLLASLTPENPSPEPKPAKKTKKAKTSTNKPLLGILFDNYEKTEDILTHAAICYLLKNGCKIPTKLEEPQEFAKKRRKAEIKVERLQEQLNSRKPKGRDLTGEKWLQTLITASTTAPENEAQAKSWQNILLTKSKSIPFPVAYETNEDLTWSKNDKGRLCVHFNGLGEHEFEIYCDQRQLKWLQRFYEDQETKRASKDQHSSALFTLRSGRIGWQEGKGKGEPWNIHRLNLFCTIDTRFWTAEGTEQVRQEKATEIAQTITKMEQKGDLNDKQQAFIHRKHSTLARINNPFPRPSQPLYQGKSHILIGIAMGLENPATAAIINGTTGEALAYRSIKQLLGDNYQLLTRQQKQKQRQSHQRHKAQKNAAPNQFGESELGEYVDRLLAKAIVALAKTYQAGSIVVPELGDMRELVQAEVKAKAEAKIPGCIEAQEKYAKQYRVNTHQWSYGRLIDNIQAQASKIGIVIEQGQQPIRGSPQEKAKEMALLAYHSRSKS

SEQ ID NO: 31 - protein, 578 aa, unidentified
  source 1..578: note = MG64-6-B transposition protein
MKKLFAQDVNIDTEVISNQIPTSDPSQSNLIASELPEEARPKLEVIQSLLEPCDRVTYGERLREGAEKLGLSVRSVQRLFKKYQEKGLIALLSGSRTDKGEHRISELWQNFIVKTYQEGNKGSKRMSPKQVALKVQAKAGAIADDNPPSYRTVLRVLKPILEKQEKAKSIRSPGWRGSTLSVKTRDGDDLDISYSNQVWQCDHTRADVLLVDQHGKLLVRPWLTTVIDSYSRCIMGINLGFDAPSSQVVALALRHAILPKRYGTEYKLNCDWGTYGTPEYLFTDGGKDFRSNHLAEIGLQLGFVCKLRDRPSEGGIVERPFKTLNQSLFSTLPGYTGSNVQERPEDAEKDAQLTLRDLEQLIVRFIVDRYNQSIDARMGDQTRYQRWEAGLQKEPDVISERDLDICLMKMSRRTVQRGGHLQFENVMYLGEYLAGYAGEVVSFRYDPRDITTIWVYRQENDREVFLTRAHAQGLETEQLSVDDAKASAKRLRAAGKTISNQSILQETIEREVLAERTKSRKHRQKEEQSYKRSPSAAVMVEVESEQLEIESSNEANANSVSADIEVWDYDEMREGLGW

SEQ ID NO: 32 - protein, 284 aa, unidentified
  source 1..284: note = MG64-6-C transposition protein
MIKENSSQEKPASQIAEELGDFKVDSQLLQIEIARLNKKSIVPLEHIKDLHDWLDEKRKARQSCRLVGESRTGKTVACEAYTFRNKPKQEGKQAPTVPVVYIMPPAKCGAKELFREIIEYLKYRAVRGTVADFRSRAMEVLKGCEVEMIIIDEADRLKPETFSDVRDINDKLGIAVVLVGTDRLDAVIKRDEQVYNRFRASRRFGKLTGEDFKRTVEIWEDKVLKMPVASNLTNKEMLKILLKATEGYIGRLDEILREAAIKSLSRGFRKVEKAVLQEVAREYS

SEQ ID NO: 33 - protein, 167 aa, unidentified
  source 1..167: note = MG64-6-Q transposition protein
MTENDIQPWLFAIAPLPGESLSHFLGRFRRRNHLTPSSLGQIAKIGAVVARWERFHFNPYPTQQEFEALAEVVGVEVERVWEMLPPMGEGMKCEPIRLCCACYAESPCHRIEWQFKSVWKCDRHQLKLLAKCPQCEARFKIPALWENGRCDRCQITFGELAAYQKSA

SEQ ID NO: 34 - protein, 713 aa, unidentified
  source 1..713: note = MG64-7 effector
MSQITIQCRLVASASTRQKLWKLMAEFNTPLINELLILAYQHPDFKTWQHKGAIPAGIIKQLCEPLKTDARFVGQPGRFFASAIATVSYIYKSWVKVQKRLQLQIDGKTRWLEMLNSDTELVEMAGVALDTLRATATELLNQLNPQPTTEESPKKKGKKAKKTQQPQGERSLSKILFDTYRDAEDIQTRCAISYLLKNGCKIRSQEEDSKKFAQRRRKVEIQIQRLTDQLASRVPKGRDLTATKWLESLLEATRKVPKNEAEAKSWQDSLNRQSSTLPFPVAFESSEDMIWFRKLKLNNIPIKLWTILLYIDYLIVILFVRDSLQNEALWFKNFKINNIHVLTKLWMILLNINSFAGVLYLGGVLKKYQQRIYVHFNGLSDCTFEIYCDSRHIHWFKRFLEDQQIKRSSKNQHSSSLFTIRSGRIAWKSAQGKGKPWNVNRLMLYCCVDTRLWTAEGTKLVVEEKAEEIAKTITRTKEKETKEKVQLNDKQLAYIKRKNATLTRINNPFPRPSKPLYNGQSHILVSVSLGLEKPATLAVLNAMTGKIIAYRSVKQLLGKNYKLLNQQRHQKQALSHQRKIAQTLAAPNQFGDSELGEHIDRLLAKEIIAVAQKFNAGSIVVPNLDNMREQVNSEIQAKAEEKCPESIEAQKKYASSYRRSVNQWSYRRLIDCITNQAAKAGIVIEENKQPIRASPQDKAKQLALSAYHARKKS

SEQ ID NO: 35 - protein, 587 aa, unidentified
  source 1..587: note = MG64-7-B transposition protein
MKDAESATNSPMTHASIADAENGKAEANIIVSELSDEALLKMEVIQSLLKNSDRSTYGELLKQSAEKLGKSVRTVRRLVDKWEKEGLAGLVQNQRDDKGKHRVDKYWQEFVLTTYKENNKGSKRMTRQQVFIRAKARADELEIEPPSRMTVYRILKPIIDKQEQAKSIRSPGWRGSRLSVKTRDGKDLQVEHSNQVWQCDHTRVDVLLVDQHGKILSRPWLTTVIDSYSRCIMGINLGYDTPSSTVVALALRHAILPKQYSLEYGLHEEWGTSGLPQNFYTDGGKDFRSNHLQQIGVQLGFVCHLRDRPSEGGSVERPFKTLNTELFSTLAGYTGSNVQERPEEAEKEASFTLRQLEKMLVRYIVDNYNQRIDARMGDQTRFQRWESGLIAMPDLLSERDLDICLMKQTRRQVQRGGYLQFENLMYRGELLAGYAGESVVLRYDPKDITTILVYRIEEGKEIFLARAYAQDLETEELSLDEAKASSRKVREAGKAISNRSILAEIRERETFPTQKKTRKERQKLEQAEVKKAKQLTPAETEEEIIVVSIDAKPTAKNPLESELCTESGEPDMPEVLDYEQMREDYGW

SEQ ID NO: 36 - protein, 278 aa, unidentified
  source 1..278: note = MG64-7-C transposition protein
MVAKEAQEVAKQLGDIPVNDEKLQAEIHRLNRKGFVPLEQVQTLHDWLEGKRQSRQSGRVVGESRTGKTMGCDAYRLRNKPKQEAGKPPTVPIAYIQIPQECGAKEFFGVILEHLKYQVTKGTVAEVRDRALRVLKGCGVEMLIIDEADRFKPKTFAEVRDIFDKLEIPVILVGTDRLDAVIKRDEQVYNRFRSCHRFGKLSGEEFKRTVDIWEKKVLQLPVTSNLSSKTMLKTLGEATGGYIGLMDMILRESAIRALKKGLQKIDLNTLKEVTAEYR

SEQ ID NO: 37 - protein, 167 aa, unidentified
  source 1..167: note = MG64-7-Q transposition protein
MESEHIKAWLFQVEPFEGESLSHFLGRFRRANDLTPGGLGSQAGLGGAIARWEKFRFNPPPSHQQLEKLAVVAGIDAGRLVQMLAPAGVSIKLEPIRLCAACYAESPCHKIEWQFKETRGCKHHKLRLLSECPNCGASFKIPALWVDGWCHRCFTLFGEMVNHQKPC

SEQ ID NO: 38 - protein, 739 aa, unidentified
  source 1..739: note = MG108-1 effector
MSFQTVRCVLCASEEMRQKVWLWTEKYTLLVNELLDQVASHEKFSEWQAQGSIARKEVEILLKSLKASDQFKGLPGRFYTSAVLMVQYTYLAWLALQKKRHFKIIGKQRWLEVLEADLEMASNTDFEWQEIQSRAFELLAQTKKSSDTVSAKPVKKGAVNQEAAKIQKQTPFNALLERFSSEKDLLNRRAIAHLLRNNLQVEENEESLDKLLFRFEKKRIEIERIEKQLESRRPKGRDPVGTRFMESLEEATALPEHTKSEEIEAELNGWKHQKQVRSLNNLPYPVLFVSITDLIWSLQSNENEQESVKGNQVQPARRGKCKKKQKRSSDRICVNFNGFKKFLGETQGVFKIQCDRRQLPIFRQIVSDWRAYQQLPDDEKFGLGLMLLKSAQLIWKKDQQKLYKKKAKNNSTQHETASGGQLVHPWQTHRLYLHCCINPDLLTAEGTEVVRQKKLPATAKKLEGSKKRQAEVAERIQTSDLGEDSTMLKKLHTNLVNRISANATSLNRLENATPQRPSKPAYHGQPHIIVGVSFSREQLVGVAVVDTRTGRVIEYQSTRLLLSNPAVKAKRGGRTVTQLRLEKYRLVNRRQRQQQQNAIRRSKEQKQNRYARSKSESNLGQYLDRLIASKVIKLAIKWQASSIVVPNLGDIRESVEASLKAEAERKFPNEFERQKKYAKHIRTSFHTWSYGRLTECIRSCAAKAQIPIEQGHQPAQGDLKEKAMQVALHAYYTRQSPKG

SEQ ID NO: 39 - protein, 302 aa, unidentified
  source 1..302: note = MG108-1-C transposition protein
MSQLQFAEPDRPHSSNEETLFLEEPRRSIEAQAEVERIGKADTYLVLDRDIALFDWLDDQRDTKLCGYIIGSQGSGLPKACQFYRMKHVKRRGRLLQVPASVIYAEVKQHGGPTDLYHAILEEIGHPLAKAGALRDLRSRAWGTLKDYGVKLLIVGNADYLKLESFNELIDLFGKLRIAIALVGTDTLAETLDRRGSAYRRIHDAFLESFDFLNLTPEDIREVIEHWENQFLPTHSRLNFARIPDVRKFLELKSKGRIEPLYDLLRKIAILKIDEPKLELNSTNLADRLGKRNAPKDRISTR

SEQ ID NO: 40 - protein, 650 aa, unidentified
  source 1..650: note = MG108-1-B transposition protein
MFRTSADSNENSASSPISELSSPGLVEAGSFKLLQDISPEVQQEMDLIDAIVQASNKCDRKAAIDRACAVLNRHPRSIMRKVQRLRQEGITSLAKGRKDNGQFRVSEQWVNFITKMYARGQKDSLRFSPHKVFVRLTVFAAQGNRLRESKYSDLFKDYPEVLKDLIQGKHPSHPTVYKIIDNYLLQKNTKARHPGAFPGKLVLQTTEGLISLTHSNQVWQIDHTKLDILLVIKVEEDDKVEHEGKKITARPYLTLVVDSYSSCVAGYYLGFEPAGSHEVSLALRNAILPKQYGQEYKLQAKWNIYGVPDYVMTDRAKEFKSEHLKQISFQLGFQRRLRAFPSAGGLVESHFDKINKEYLSEKPGYTGSSVEERPKEAENAACLTLDDLERELVRYFVDHNNQHFYSEDKLTVSERMSKPKRFERWQDGLLVEPEILNERELDICLMKSIPRKVEKYGCVRFETLRYQGECLANKAFIGRDITLRYDQRNIATLLVYSYSTTSQIQEFIGTVQAIGLERERFSLGEWEALKQKMRDRDKEIDNSILMAERMGLIESTKEQIKSSRSKRQRIKKAHTEQERKANRSKVVELYPERAIEDTTVIQENVESTPNSSIQKRVESTQEQQIQQAAPKTRRVRTVAPDWNQIKQDNW

SEQ ID NO: 41 - protein, 200 aa, unidentified
  source 1..200: note = MG110-1-6 effector
MKLFSVKITYLPEKCDHALLAGRCIKVLHAFMSRNGQFNIAVAFPRWSENTIGNQLVFVSPDYKFLDMLLEQPYFRMMIENGLFETGTVVDLPVSDSYVKFVRNQSIDKMTPAAKARRLRRAKKRALARGEVFDPIAPRSKDVDFFHSIPMESSESEMSYLLRVQRYEVQQANPVASFEVCSYGLSTNESHQALIPSSVT

SEQ ID NO: 42 - protein, 342 aa, unidentified
  source 1..342: note = MG110-1-7 effector
MQLPNQLSYKRSINPSKAIFYYRRDEDLYPLPVERIKIRGSKSGFSEAYTAKGIKESATIHSLATGNPHTIDTCYLPPAADSLVCRFSLRVSANSLFPDRCSEIAFKDTVSQFLNSYIAKDGFKELAIRYSKNIAMGTWLWRNKEGNTFDVLVRTSQGNEYKFKNAHMLFWDSVWPDESSELLALLSDELAVALTKPRYVWHCDVWAEVKMPFCSEVFPSQCFVDHDDKQSASKVLLTTDIDGVMTACYNADKVGAAIQMIDDWWDESCDFPLRVNEYAADHENLIARRHPSTERDFYQCLQNLRGYTDKLNKVKSVDDIEPDAHFVASVLVKGGMFQGGKS

SEQ ID NO: 43 - protein, 682 aa, unidentified
  source 1..682: note = MG110-1-8 effector
MSLQDTLQAVKALKAKERYDALKKSFELYSEHVDVTGSELACLTILVNLVSKDAVELEGLRHAKHVLKSDEFWGKFSKVASQLHTHNLKWPDSRVNLKHHIRALPEIGVLPKFGWSGNSGDYRFGRLLTSTFIWQGLEHSLISVWLGDFVAWRKAAYKLGITKVFWYQIKRDLEDLFQESHFPDVVDSYSPELQFPYKDQYLSVTPVASHETQLAIQHVTGLPMHSLSFPHPSALGVLCGSLGGHVRLIRLSPLGNQNVQRSSLGSLSLKNYLNPYVLTAIPAINLYRRIVDVRTYSSLRLKRRARLNALSTLDGILAEWVAPLVQVKLSSNVDGRIDGLGAEEQEFVESSSVDIEEFSRYLNRKLHGMLELGKYSRKFSYHQRLVGVTQKRLASLLRRLLLAETQSESNNTTFIILKSLRINEANGLNNPYVVGMPSIIGLYGFLHRFECQLREIYADISVDSFALHCGEYSYHASNNLPAPSIPDKEMRIKRSGITPEFKFDGKFSIIVKLHCLTDNAVLDVEQIKAAVPERLWGGSVHPPYLYENTEWAAIAYGSADLERYLIRNLFFGNWITPEEEDGFELRKHIEKLNGKNELSLCLVGYKLLEKVKPRSVVSGIHAFCEPVVDLCCLKQTHKVIKPTKSIEQELFWRYVPVSQNCTTLRVSPVCGETHAASQSAEL

SEQ ID NO: 44 - protein, 383 aa, unidentified
  source 1..383: note = MG110-1-Q transposition protein
MGFLPNSIHLYPDETVESALLRLCKANHFERYIDLSIEIRSWLEEHHPTIAGAFPVALDAVNVYHAKQSSAKRVQSLQLLEQLVGLPRFSLLDISFKHTNAVDAGHFAEVRYKQITIPKSFVRASSVPVCIACLRESNYVRFDWHISKVTCCEKHKVKLLSNCPACNAPLNYMISEDPSHCVCGFNLLETTNSEKGADDWRRRISFDQQGSLSISEQLALLLFLERYFSHVELGDFIGNYKSLIDKHLQILTSKNILLATEKINRVSFSSLTDNFLGDVAQIGSLPNFIKQHIANVVIELALETPRSTIANLGDSLVSMRDAALITGSTIDDIFRLYESGILVVGKRIRNEGRIESFNPLFRLRDVAAIALSYSRYGYSQSAW

SEQ ID NO: 45 - protein, 334 aa, unidentified
  source 1..334: note = MG110-1-C transposition protein
MGLTDADKAKLREFKDCFCPYTPVTTVLNDLESLYQSSEIGGEQLSMLLRGDTGTGKSAIIHHFCNIKNGINLESIPVLLSRVPSKLTVEDMTRQLLSDLGVFGSSTHRAKNAQSDAHLTNRLLDALKVKNTKMIIINEFQELIEFKGARDRQAIGNRLKLISEEAAVPIVLVGMPWIDEILNDSQWASRLATRTHTLQYFSLSKRIREYFEFLEAIETYVPCEIETSIIDFEISIALFAASCGEMRQLKAILTEAIKLCLISGEPFSKQALSHSFANLYAGIENPFDMPKEKIKVREVEMHSRYIRGDSTHRAAIEPRRLSDFMSLTQILSKK

SEQ ID NO: 46 - protein, 601 aa, unidentified
  source 1..601: note = MG110-1-B transposition protein
MFDDEFDHDPLKEDVESSNRQSDEASYLSPDLDSYPIKSSEEALVRYELVQFIRERTSKGWTQRNLNPLISEYFSTHRSLERPSWRTVVRWHKKLLEQGDTPVSLIERHHNKGNRNRKLLLDHERFFEAAIDSFLKAKRPSVATAYRYYKDQCLLQGNNINPMSQRAFYDRIDKLNSYEVAVKRFGKYKADIMYGHKGSTLKPERVMQRVEIDHTVLDITLLDDDTGMPIGKPSLTLLKCALSGCIVGYNLTFKAPCFASVAKAICHTLLPKTSSLDIWGIDWPCYGKIEVLVVDNGVEFWIPSMEKMCFELGINVQYNPVKKPWLKPFIERNFKTINSLLLDELSGKTFGNIYVRGDYDSVKNAEIPFGVFVYVFEKWAAEVYNCSPDSQGMKVPSIIWQDGIEKFPPARLSDSDIRELPKITGFKESRKIQSSGITYKHLRYDSTDLAEYRKQYWSNTRKELVTIKIDVDDLSKIFVYLPEIEKYLTVPCVDQEYTKNLSLDQHLITRTYTKKRNKLLGKSELELAKSRDEIRDAVEQHDKKAATSKKVTTSKKVAQFKGYSNESVKNKVAKPISSDDKSRAEHAEMSELEALWNSFRK

SEQ ID NO: 47 - protein, 207 aa, unidentified
  source 1..207: note = MG110-1-A transposition protein
MYNRNLRKPSPNKNLYKFVSRKNHSTVMCESGLEFDACFHLEFSPSIASFESQPTGIEYQADNKIRRYTPDFKIVKNTGEIEYIEVKPERIHSTKKFRDEFEYKRAAYRALGFKLILVSEKQIRSEKLLSNLKILHRYASTNFSELHKLALTHIKKFKSLSIRELAIKLGIRICDCIAACALLIGIGAVKADLESDFLCEHSLLNEA

SEQ ID NO: 48 - protein, 215 aa, unidentified
  source 1..215: note = MG110-2-6 effector
MTDKRYFFAIHYVPAKADCGLLAGRCISTLHGYLLNHAVTQIGVAFPCWSNKSIGRTIAFVSEHSSHLTQFRERTYFQTMQGDGLFELSPVLEVPDDCAEVRFIRNQNLAKLFVGERRRRLLRSKRRVAERGELFVPKAPAINQEIAPFHCALIQSASNAQSYVLHIQKQSCDSEDSTNTYSRYGLASPNSYRGSVPELSSSISTLFPDSLYLKR

SEQ ID NO: 49 - protein, 345 aa, unidentified
  source 1..345: note = MG110-2-7 effector
MELCKHLSYSRSLSPGKAVFFYKTADSDFVPLRVETARVVGQKSGYSEGFDANFQPKNVERYELAYGNPQTIEACYVPPNIVELYCRFSLRIEANSLSPDLCSDPGVVKTLKQLAGLYQEHGGYIELARRYSRNLLMGTWLWRNRHNQGTRIEVKTSQGSHFAIDDVRHLDWFSEWPALAEAQLNQLAAEMAIALSRRDMFWFADITAVMKTGFCQEIFPSQRFTERPDNHDEPSKKLATVECLDGQLAACLHAQKVGAALQQIDDWWAEDADQPLRVHQYGADSKNLTSMRHPVSGLDFYHLLGRADEFLELMRSSPASSPLPGEIHYLMAVLTKGGLFQKGKG

SEQ ID NO: 50 - protein, 710 aa, unidentified
  source 1..710: note = MG110-2-8 effector
MPLHELMNLHDQAEQDKALRRAFAPYTPDIELEGEEYRALVVLVNLTLKRDQAVDLCDESQAQRLVNDDKHLEHCVHPVAWLHSHNLKYPDTRVSGQRLVIDAPPLIPGVVTSAGLQNRLGWANNSADINYAKLFCSSFLYQGLSSNLALQLVADVPAWTGAFRQLGVANTAIAALQAQLAHHLAATAIPSEVSVYSKQVRFWYQGDYCAITPVVSHALMAHLQHMIYEKRCSHLTISHDHPSSVGNLVGAVGGKISVLNYPPPISIPKRRHFSQSRTQRLNEGRSLFDRGMLYDRIFPQALEHLITPAGLTRRQRKQSRLSAMRYLRRQLVAWIGPVIEWRDEIQLLPNIELPITPERLEWKIVTSPVAELPDLATELAGVFHLELQTHHATRRFAYHPELLIPIKSQLRWLLNKLADDENRPSPQHAATSCYLHLSGLRVYDALALANPYLCGIPSLSALAGFCHDYERRLTALLKRPVRFTGVSWYLSHYHLLSGKSLQEPLSPMHAREVSAIRRPGLIDSKYCDLGMDLVIALQVDDEHPLPSANEQDLLQAAFPSRFAGGCLHPPSLYEGKPWCSLYTNRNELFNTLSRLPRTGCWVYPYQSRISNLDDLIDTLSMDRRVRPVSTGYVFLDEPRVRAGSVEKCHVYAESALGLALCVNPVEMRLYGNNHFFNHGVWQLTAINRAILMTGAGNMEQRHGAMQTSEL

SEQ ID NO: 51 - protein, 409 aa, unidentified
  source 1..409: note = MG110-2-Q transposition protein
MRLLIRPEPYPDESLESYLLRLSQENGFERYTLLSGAIRDSLLVQDHEASGAFPLELARVNVFHAQRSSGLRVRALQLFAQLTDLDPACLLQLSLMHSGLHFGSGHACVHRAGVDTPLGFIRTGPIPVCPACLRESAYIRQHWHYSPYLACHQHGSQLLHLCPSCGEALDYQHSESFTHCRCGFDLRTAITQPASAEAQQLSTLICGARFESTNPLLSSEHPSLMFGALYWYWLRNKRADAGQPEFGTLTMAIDYFSAWPANFHHELQQMAQKALQTQTRLLNHTAFREVFGSVLTDCRQLPTRDSHRNFILRGLLDYLTELVCANPKTRVANLADIQLSAIEAAALLGTSVEQVFRLLQDGYLTPAHRRIHGGLKPHEPLFHLRHVIECRQAHRSTFNDAYHPYLPAW

SEQ ID NO: 52 - protein, 335 aa, unidentified
  source 1..335: note = MG110-2-C transposition protein
MELSSTDADKLKSFIDCYVETPLLRIIQDDFDRLRYDKQFAGEPICMLLTGDAGTGKSSLLRHYMAQSPEQSGQGFVRKPLLVSRIPSKPTLESTMVELLKDLGQWGSEYRLHRSSAESLTEALIKCLKRCETELIIIDEFQELIENKTREKRNQIANRLKYISETAKIPIVLVGMPWAAKIAEEPQWASRLMVRRTIPFFKLSEDAESFVRFVMGLARRMPFATPPKLEAKHTIFALFASCHGSIRRLKHLLDESVKQALAAHSETLQHEHIAVAFALFYPDQINPFLQPIDEIQTCEVKQYSRYEIDAAGKDEVLIPTQFTDKIPISQLLKKR

SEQ ID NO: 53 - protein, 630 aa, unidentified
  source 1..630: note = MG110-2-B transposition protein
MMCAQQSSEHPSGLFEDEFALPEPAASSTPSESTVTGISATFDSFPDELKNEALHRLKYLQWIEARLIGGWTEKNISPLLVEAASILPPPVPNWRTLARWRKNYIQQGKKIIALIPRHQAKGNSQSRLPLSDEIFFEEAVHKYLVDEEPSIASSYQLYKSKVELENKTIVQNPIKILSYKAFYDRIKMLPAYQVMKCRKGLHLANAQFKAIGSHKRPTRIMERVEIDHTPLDLILLDDELLVPLGRPCLTLLIDCYSHCVVGFNLNFNQPGYESVRNALLNSIPQKNYIKDKYPVIEHEWPCYGKPETLVVDNGVEFWSNSLEQACLELGINIQYNPVRKPWLKPMIERMFRTINCKLIDPIPGKTFSNYLEKGEYNPEKDAVMRFSVFLEIFHQWIIDIYHYEPDSRHRYIPILSWQYGFDRLPPAKVTGEDMAKLEVILSLCIRCKHTRGGVEHLYLRYDSEEFASYRMKYPSKTDGKQYVLVKLNPRDISYVYVFIDKIGEYIRVPCVDSEGYTRGLSLQAHKINVKLHRDFIGKKIDVVSLAISRAKIESRIIKELTEVRQTLKKHNIKGINKIAKYRDIGSQTAANLLSSTQTPENTNDNPVQPKTDQLPLEDDWDSFTSELEPY

SEQ ID NO: 54 - protein, 207 aa, unidentified
  source 1..207: note = MG110-2-A transposition protein
MYRRHLKHSRVKNLFKFVSAKMNTVFTVESSLEFDTCFHLEYSPAVKTFEAQPEGYYYIFEDRECPYTPDFRVVDERGNPYFIEVKPSAKVATPDFLQRFPIKQQKAIELRSPLKLVTERQIRVDPILGNLKLLHRYSGFQSFTPLHIQLLGLVRQLGRVSLAKLSGSTGAPPEEILATTLSLMARGLIHSDLTEREMGLSSVVWAI

SEQ ID NO: 55 - protein, 400 aa, unidentified
  source 1..400: note = MG64-8 effector
QFNIENETGSAPNAKKRKKSKSANDPNLMSYLFKAHKATEDILSHCSISHLLKNDCKVSETEEDPDKFAHRIHRKQKQIEQLDAKLNARLPKGRDLTGEEFFRTLETATHQISDNVIQAREWDAKLLTRPATLPYPIIYGSSTDLRWGKTKTGRITVNFNGIDKYLNAADPNLKEWFKTHEEHPFQLYCDRRQLHFFQRFLEDWQAYQANKDTYPAGLLTLSSAILIWTECEGKGDPWNVNHLSLHCTYDTRLMTAEGTLVVQQEKSAKAAKNLEREKSDPRNQSTLNRLNNLPARPSKQPYQGNPEISVGLSIGLTNPITAAVVNVTTGEVLTYRTPKTLLGEQYRLLNRHRDRQQQNTLQRQKNQKRCVRYQPSESELGEYVDRLLANKIIQLAQQYQ

SEQ ID NO: 56 - protein, 748 aa, unidentified
  source 1..748: note = MG64-9 effector
MGMKTIQSDLIVRLDPNPRRGKKNLDDLSEFDAVAWCRLCERAYQHTLLIDQISEQIKLNPDVIAWIYAVQNSEDQNSADMDTDTDSEQGYSWLPDGLINGLCALLKNTPQFEGMSGRAYTSATDRVEENFKGWFANHQKLIRQIKGKQHWLTVLESDAELAENSNFNQSEIENQAKQILNEIEADNESKDESVNDNRRVFSILFDRFEATEDVLNRRAIIHLLKNGGKVRWEPKKSRNRKSQKHPSKPMTFEERLVAKRIEIARLEKQLLSQLPRARNLFPDIAFEESLAEVVALPKLSDEEQVEAEQVESEFYNWHESITFKLLEFLREPKSLPYPISFGYEDVRSWQINQAGKIFFKLNGWGDLIFEVRCHRRQLPLIKSFLKDWQTKEQCQEGDQYSGSLMLLRSIELVWKPKPVNEQNDIQLCSQCEVFQQYPSKGFWSECKLSIHWSYDSDALSKQGLEKVRQRKLEPQLEKLRKKQEELEEKQQLLQSIEEVPEALRSKAQSKKMRSLTKAIQELQDDLAKPRPKLDCLQNSLLFDRPDRPLHEGVPNIFVGVLLDLDKHLVVTVVDAMRRKILAIRNARSISKEGYDLLQSYFRQRREHSKERQIDQKAHRHVHQTESNLGQHVARLFAKGIVELGQKYKASTIVIPETDGWRDRLYSQLVASAAIKCKGVKKAMTLYTKQHGEKLHQWDYNRLSQAISDRAATDGLKVMRQNTVYEEDAFQQAANLAIAAYDSLNSVEK

SEQ ID NO: 57 - protein, 422 aa, unidentified
  source 1..422: note = MG64-10 effector
SWQLNQEGKICFKLNGLGDYLFEVRCDRRQLGIVKYFLQDWQTQNKNKNEYSGGLTLLRSAELLVKPKLGKQNAKLPPIHDRQAVVTAYKLSLHCTYDTDYLTHQGLECVRQRKIANQLKGLTDKKAKLTKQQEQLQQLEQEMQQEQIGTSAKRSKRHAQRLKQIEQLKQSISKLQAAIQAELERPRPKLERLQQSQLFQRADRPLYAGVAHLFVGVCLDLDQHLVVTIVDAMRHKVLTKRTVKQIMGEHYPLLQRYRRLKQQHPKQRRQDQKVGRHNHLSETGLGEQVACAIANGLLSLAQQYKVSTIVLPETKGWRERLYSQLVARAKIKCNGSKKAMARYTKAYGKRLHQWDYNRLSRAIETEAQTVGVTVIFQRLEFQANAEQDNQPADEADEQDNQRVNPFELALQIAIAAYDSLQA

SEQ ID NO: 58 - protein, 404 aa, unidentified
  source 1..404: note = MG64-11 effector
MAESTLWEKSEPEPLAQLPTLPHTLPYQIRFESSDDLYWSLLPKGKQEDAEKNEGKFEQAEPLPRPAPKRKRRKRRKIHYQASICVRFKGLAEHSFRVQCSHRQLPIFQQCLDEWQLLNALSDDVKFSLAVFPLRSARLVWCKDKQASKHSKNPKAEWNQYRLYLHCTIDRRTLTAEGTEELRQQKLVEAQKQKEASVKKQKPPKIPAPLTEEQRQAKNQAEALKRNHSTLLRLQNPSPPRLSKPCYVGQPHLVVGVCFSRRDRASAIVYDIQQHQEIEYVGVRELLTDESAEEHYRHFSSNPQRKGKRSLEQMRLEQYRLVERFYREQEKYQQRRAEAQECGLELEDESVSNLGEYLDRLLAARIVEFAVKQQASSIVIPELGNIRESIECEVQMQMENRGFE

SEQ ID NO: 59 - protein, 731 aa, unidentified
  source 1..731: note = MG64-12 effector
MASGILRTIQARLVPSEWMPLDQKASEDSGESQPVPIAVVSSFVEDCTLQLGLTSTRLCRALNECINQHSDFEVWKEGGSIPPKALKEIWNSIRTMPPYQDLPERFLRSAWLRVEGIYASWFAIRTRLAAKQDGLNRWLSIVRSDEYLTKTCDCSLEEMQARATQLLTEPSGSSLSALFDAYFALVKADGSLLDRCAIVHLIKNGCAVASKPEDLKKFAATCQKKRQQVERITKQLAAPAPQVRDLGEAAEAALTDSVERIPSNNADFVAQLAALQRKPIPLPYAVVFYSNDDLEWHQIKRRNSATEKIEERIFVKFKGLNKYLRNRVKYNLEAQLAQILESSGLKLDDISWKIIRRKTAQSTVKYISFKLKTPDPTLKQHLKEQFEQIDLEDKYNLQKEYGFELCCGYRQLADFQTFLSDWQLYCTNRDRYSVSPFALRSAALDWQKEVKQGKSKLQVYLRCTLDQQELTAEGAERARAKAATELNKKIAKLEAAQEQGEVLSEEQQRDLERARSQLRGAEHPYPRPSQPLYEGNPDILVGVCLSWEQVATVAVVNWSTRQVLVYRTIHQLMGEDYPLLSSYRHEQPRNANKRHKRQKRGQSSNISEASKGKHIDRVIAKRLVEVVVEFNAGCLVLPNLNGLRENLQAELMAKAAWKHPGDVAKQKAYLKQYKKNLHCWPYRRLAQFVKERADKIGVPIELGQQPREGDFKEQAVQVAWSFHEAQQADEG

SEQ ID NO: 60 - protein, 741 aa, unidentified
  source 1..741: note = MG64-13 effector
MAIKTLRSQLCATEETREYLWHLFITYTLLINQLLERLPKDAKFSDWRKEGRVPRKAVAALCNKVLEEDGSLKGLPARFYTSAVLCTSYTFASIFAIQNKLRARCEGKQRWLNVAEHDLELAETTAFSLEEIRERAAQVLGQAESERQECLDEEEEPSNIPSIMSLLFKLLDKSNDLLSYRAIAHLLRNDCKVSYEEEDPEKLALRLSKKRIQIERLQEQLNSQLPIGPDPTGERTNQFIEEAIFLPRDSDSSENRIKEFETWEDLLPERIANLSTQLETLPYPLLLGSNDDVSWSWETISTAASSSSTSPNPSPKRTRTRRRRRKAKKRICVSFKGKGLSQIRLRLYCDRRQLPIFRQFVEDSKANKARNKKEKFSLALYPLRSATLLWAEDPKLSTEKNRDKPWNSHRLYLHVTVDTRLLTAEGTEQVRQEKIELMHKFLKGLVEAEETQEANEDEEAFLVAQKNRSRATKKNQTTLKLLESNPPPPRPRQSAYRGNAEIAVKVAFSREHVVGVAVSDGQQPILDYRDVETLLIDSHLELLEQRSHKLRNQPERLRKVKLGDSKKAKKAKRTRYKPKISTRQLQLQPYRLLKRWHRLKRKNVTERRAEQKHGLYRQSQAETNLAHQINHILARNIVDLCQKWSAGTIILPEFGDLRESIESEIQAKARRKYPDDNVERQKQYAKEFRMQFHRWNYKHLSECIRLRAAKVGIACIAGQQPRLGTLREKAIAVTAIPPKPK

SEQ ID NO: 61 - protein, 642 aa, unidentified
  source 1..642: note = MG64-14 effector
MSIITIHCRLIASEPIRRHLWQLMSRSNTPLINDLLKQVSHHADFETWQTRGTVPSNAIRDLCEPLKEVYPGQPARFYASAILMVTYTYESWLALQQTRRRRLHGKQRWLNVVKSDAELLGLSGSTLESIRQRAQDILSQLSTEMETQSAPNPKKRSQRQGQTHSSNDASLMPRLFTAYDTADDILSQCAIVHLIKNGCKISETEEDSEKFAHRIHRKQKEIDQIEAQLQARLPKGRDLTGDVFLETLAIATQQIPETVIQAREWQAKLLSRPASLPYPMIYGSSTDVRWGKTANDRIAVSFNGIDKYLKEADPEIQEWFKLHKEYPFRVYCDQRQLPFFQRFLEDWQAYQANKDTYPAGLLTLSSATLAWREGEGKGEPWEANHLALYCAFDTRLMSAEGTLEIQQEKSTKALKNLTHDNPDPRNQSTLNRLKNVPDRPSRKPYRGNPEILVGLSIGLADPVTAAVVNGRTGEVLTYRSPRTLLAEHYHLLNRYRQQQQQNALQRHRNQKRGVTYQPSESELGQYVDRLLANSIIQLAQTHQAGSIVIPSLTHVRELLASEITAKAERKSRIVEVQDKYAKEYRIAIHRWSYNRLINAVCSKAQQFGMTVESGFQPLQGNPQEQAKDIAIAAYYARTIAIK

SEQ ID NO: 62 - protein, 828 aa, unidentified
  source 1..828: note = MG64-15 effector
MSFKTIRSLLCATEETREYLWHLFLNHTLLINQLLEKLPQKTAFAKWRKSGRIPRKAIVAACNTSLQEDESLEGLPARFYTSAVLSVSYTFAFIFAIQNGLRAKVEGKQRWLNVAEKDLELAQTTSFSPEAIRAVAAQVVDQAEAERQREFEARENRTGQPLPLMTVLFKFWNKTKKPLRRRAIAHLLRNDCQVNSEAEDPDKLTLRLSKKRIQIERLQKQLDSQLPTGRDPLGDRIEKFIEDAIAFAEHSTFVPTQFWLKWHRLLLDVQSSEPMLDLWFFGWTYYRLNAIAEFEAWEQSLSERTANLSTQLKSLPYPLLFGSTDDLYWSWEKQVQPKTPQPAPAHPQASADPTIKRKPKRCRTRKRKKKSTPRICVSFKSRGLSHLCLRLYCDRRQLPVFRQLVEESEANKARKKPDRFSLALSPLRSAGLMWVEDPQQLHKKKHWKLKNLWLKWFCEMSSQAFPDEIREQWFQSLVYLCLSLPWQTAAIDPRLLTAEGTEVVRQEKITLMGKFLEGSKKADEKQAIEELSKKKQAKLLTAEKNRCKVTKKNQTTLTLLQSNSPLPRPSRVAYQGNPDIAVKVAFSREHIAGVAVSDGCQPVLDYHDIKSLLVDPRVDLLEERSHKLRNQPERLWKATLATQKSKPSKSKYPRSKPKISTRQLQLQPYRLLKRWRRLKRENGAKRQLEQRHGLYRPSQAESNLAQYINHLLARNIVDLCQRWSAGSIILPKFGDLRESIESEIQAKAKRKYPDDNVERQKQYAKEFRMEFHRWNYQHLTQCIRSRAATVGVKCVAGQQPRLGTLREKATLAVSAEQRVVTAIPPKPK

SEQ ID NO: 63 - protein, 601 aa, unidentified
  source 1..601: note = MG64-16 effector
MTKLTIHCRLVAEEETRRQVWEMMAGRYAPLIATTLEQVSQHKDFPQWVATGELPTQVVKNLVNQAQSGLPARWCASAQRQVQETYKAWLTKRRKLQQKLQGQQTWLSVLRPDAELAKNAGLSLEEMKIRAQALLHREINNWFQVYQQCQDVVERSIFAYLLKHRLTVPAEPENTDKLRQKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKAQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 64 - protein, 605 aa, unidentified
  source 1..605: note = MG64-17 effector
MTKLTIQCRLVAEEETRRQVWEMMAGRYAPLIATTLEQVSRHKDFPQWVSAGEIPAQVVKSLLHSAKTSYGGLPARWYASAQRQVQEIYKGWLKRRRQLQHKLQGQQTWLSVLQPDAELAQSAGLSLPELQQRAQALLAGDTVTNWYHAYQQCQDAVERSIFAYLLKHRLTVPTEPEDTDKLKRKRRRVEIRIERLETQLAGRSPQGRDLTGIRYATALHEGEQCYWEDDADFLAWQAEILSCPDSVPPPVEYATNTDMTWHKDEQSRLWVTFNGLGKHKFKIACDQRQLHWFERFYKDQELFKAQKGQRSQALFTLRSCELLWKPGNRSGELWQVNCLYLHCTVDPRLWTQEGTAIVQQEKAQKSQAIVKELSERSDLTPGQEDYLRRHQSTLARLHTGYSRPQRRMYQGKSHLSVGISLDMDNLVTVALVDVVEQRIITGTTMKNLLGSGYALVQRLRYEKRRNAHLRKVAQQRGSKIVNHEANLATHIERLLAKAIINFAQQHLAGSLCVPTLKDIRETIQAHIQSRAEERHPNSKELQRRYAKEYRINAHRWSYNRLLELISQQAIVAGLLVEQGLQGSGGTAIKRALGVALSAYYQRSAA

SEQ ID NO: 65 - protein, 613 aa, unidentified
  source 1..613: note = MG64-18 effector
MSIKTIRCRLVASESTRKALWELMAGKNTPLMNHVLALVSSDTNFDSWRQKGEIPASIIKNYINTLKHEPQFAGQPWWCYASSEKQATRIFDNWLATQKQLQSKLNGKEQWLSVLQPDSELASQASCTLAALRRLASQINKQGLTFNQLFNLYTTVQVLKQRAAIAYLLKRQGKLEPNEEDPEQIARKRRKTEISIQRLKKQIQARVPKGRDLTGSEYTAALDECIQTAITTDEEYEAWQGKLTAKTVSVPFPLICESSEVLKWSQTSSGRIVVKFSGLHGLSFDIYCDKTHLRWFSRFLADQEVKKASGGKHSAALFTLRSATLLWRPNKHPSKGDPWSTNYLELHCTVDTRLWTAEGTEQVRREKSTEVEKILTEIGSKDSLSNNQLVYIKRKQATLTRLQGNFDRPHQSPYCGDPDISVGISMGLDKPVTLAIVNLKTGQVSAYRTTRQLLGSNYCLLGRRRKEQERASHRAHINRARHRSDYHTYKESQVGTQIDHLLACSIIEVAMQYKASSIVLPDLEYIREVVEAEIKERAERRIPDYSEGQKRYAKEYRKKVHSWSYRRLHNFIKNKCEAVGIAIEVQRQETIGTPQQKARKLVEKAYANRCFVA

SEQ ID NO: 66 - protein, 543 aa, unidentified
  source 1..543: note = MG64-19 effector
KNLVNQAQSGLPARWCASAQRQVQETYKAWLTKRRKLQQKLQAQQTWLSVLRPDAELAQEAGLSLPELQQRAQALLAGDTITNWYQAYQECQDVVQRSVFAYLLKHRLTVPNEPEDTDKLKRKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 67 - protein, 605 aa, unidentified
  source 1..605: note = MG64-20 effector
MSKLTIQCRLVACEDTRRQVWEMMAGRYAPLIATTLEQVSQHKDFPQWVSAGEIPAQVVKSLLHSAKTSYGGLPARWYASAQRQVQEIYKGWLKRRRQLQHKLQGQQTWLSVLQPDAELAQSAGLSLPELQQRAQALLAGDTVTNWYHAYQQCQDAVERSIFAYLLKHRLTVPTEPEDTDKLKRKRRRVEIRIERLETQLAGRSPQGRDLTGIRYATALHEGEQCYWEDDADFLAWQAEILSCPDSVPPPVEYATNTDMTWHKDEQSRLWVTFNGLGKHKFKIACDQRQLHWFERFYKDQELFKAQKGQRSQALFTLRSGELLWKPGNRSGELWQVNCLYLHCTVDPRLWTQEGTAIVQQEKAQKSQAIVKELSERSDLTPGQEDYLRRHQSTLARLHTGYSRPQRRMYQGKSHLSVGISLDMDNLVTVALVDVVEQRIITGTTMKNLLGSGYALVQRLRYEKRRNAHLRKVAQQRGSKIVNHEANLATHIERLLAKAIINFAQQHLAGSLCVPTLKDIRETIQAHIQSRAEERHPNSKELQRRYAKEYRINAHRWSYNRLLELISQQAIVAGLLVEQGLQGSGGTAIKRALGVALSAYYQRSAA

SEQ ID NO: 68 - protein, 638 aa, unidentified
  source 1..638: note = MG64-21 effector
MGQITIQSRLVASEATRQHLWMLMADRNTPLINQLLEQINQHPDFLTWRQKGKIPVGTVKQLCQPLRSDPRFSGQPGRFYTSAIALVDYIYKSWLTLQQRLQHKLNGQQRWLEMLKSDEELAEISGCSLDTLRQRAANLLQADNPIHPEPSTLPKSGKRRKPKQTETSALSPNISQALFFAYRETEDVKIRAAIAYLLKNGCTLPEKPEDPQKFAKRRRKVEIQIQRLTEQLEGRVPKGRDLTNTQWLAALETATTTAPQSEAEAKSWQDSLLRESSNLPFPVAYETNTDLTWFRNQQGRLCVRFNGLGDHIFQIYCDRRQLHWFERFLDDQQVQKDSKDQHSSALFTLRSVRISWQEGKGKGTPWQIHRLALQCSLDTRLWTQEGTEQVRNEKAADIAKILTKMEDKGNLNDKQEAFIKRKQSTLDRINHTFPRPSKPLYQGQSQIIVGVSIGLEKLATAAVVDASTGKILTYRSIRQLLGNNYRLLNRQRQQQHHNTHQRQIAQRQGKRGLLSESELGQYVDRLLADAIVDLAKTYYAGSIVVPKLGDVRERVQSEIQARAEQVCPDLLSGQQNYAKQHRSAIHRWSYGRLIDQIQSNAKRTGIVVEDAQQPIQGTPQEIARALAITAYQDRLKMI

SEQ ID NO: 69 - protein, 502 aa, unidentified
  source 1..502: note = MG64-22 effector
ANLLQADNPIHPEPSTLPKSSKRRKPKQTETSVLSPNISQSLFVAYRETEDVKTRAAIAYLLKNGCTLPEKPEDPQKFAKRRRKVEIQIQRLTEQLEGRVPKGRDLTNTQWLAALETATTTAPQSEAEAKSWQDSLLRESSNLPFPVAYETNTDLTWFRNQQGRLCVRFNGLGDHIFQIYCDRRQLHWFERFLDDQQVQKDSKDQHSSALFTLRSVRISWQEGKGKGTPWQIHRLALQCSLDTRLWTQEGTEQVRNEKAADIAKILTKMEDKGNLNDKQEAFIKRKQSTLDRINHTFPRPSKPLYQGQSQIIVGVSIGLEKLATAAVVDASTGKILTYRSIRQLLGNNYRLLNRQRQQQHHNTHQRQIAQRQGKRGLLSESELGQYVDRLLADAIVDLAKTYYAGSIVVPKLGDVRERVQSEIQARAEQVCPDLLSGQQNYAKQHRSAIHRWSYGRLIDQTQSNAKRTGIVVEDAQQPIQGTPQEIARALAITAYQDRLKMI

SEQ ID NO: 70 - protein, 541 aa, unidentified
  source 1..541: note = MG64-23 effector
ADSLRSDPRFKSHPQRLVASAGRVVNSMFESWLAIQKGNFARLKGKQRWLAMLKSDEELVEYSDCDLYQIRNHAAQILESVAASVPSQTSNPANKKSKQQKSDRKKSKQKDNNIPKALHGLYDSTDNVLTQAAIIYLLKNGSKVSDCEEDPEKFTQKYRKTELQVERLQKQIDANMPKGRDLTGQVWEETLEIATLTAPRNGDEAKEWQNTLLRKPKNTPYPIFYRNKEDLRWYEIEINGKARRRGKNHNSPKKQLCVTLSGFGGHALKVSCDRRQLHWFKRFLEDQQTKKNSDGQCSSSLFTLRSAQLLWHEQKGNGNSWDVHHLHLHCTVETPRWTAEGTEKIRQAERQEVAKRIESLNQKEALSPSQQKYLQRLDSQLKGLDYPFDRPSRPLYTPQPDIIAAVSMGLQYPATLIIMNSSTQEVLATRTIRELLGSNYGLLLRRRRQQQKNAHQRHKAQKRSASDQFGESNLGEYLDRLIAKEIVAIAKVHKAQSVVIPKLARIREILNCEVMSRAEQRCPGSVEIQKRYAKEYRTKIH

SEQ ID NO: 71 - protein, 614 aa, unidentified
  source 1..614: note = MG64-24 effector
MSQKTVRCRLISTEENRKALWLLMAERNTPLINEALRQLPSHSDFPKWRQKGKLPDIAAKCLIDRLKTDARFANQPVWYCISAQKHVTYIFRSWLAIQRRKQWKLEGKRRWLEILQPDTALAEKAGCSIEALREEAGEIIAEAEIVDPFRHLLAKYRETEDVREQCAIAYLLKRKAELEPEEENLAKLVERHRKTEIFIQRLEAQLDASLPKGRDLTGHLQAEALIQSIHSPLLDDSNYNTWKDALTTEPAKFPFPIMYETTESLVLSRDDRGRILLRFSGLSQQTYKIYCDKPHQHWFERFFEDQETKRVGGDQHSAAAFTLRSAQLMWVPSEKHRDEPDPWNRYYLNLSCTVDTRLWTQEGTKVVVQEKAVKTAQKLTSMQEKKSLTQTQRGYIRRLESTLQRLQNPYPRPSRTVYWGQPEILVGVSMSLDKTVTIAVVNALTEQVLTYRSAKQLLGERYRLLQRARKEIVKISHQGHRQRRKGGRRISQESDVGKYVDRLIAKAIDTLALKYRAGSIVLPNLAYIRESIEAEVQQRAIEKVPDFKDGQKQYAKAYRTQIHRWPFSRLQSAIISKAEQSGITIEIATQQISGSFQDKARELGLQAYAHRSAS

SEQ ID NO: 72 - protein, 440 aa, unidentified
  source 1..440: note = MG64-25 effector
LKNRQRLPKKAEDPEKLAQRRRKVEIKIERLRDQIESCIPKGRDITGEAWLNTLLTAANTVPQSAQEAKTWQDILLTRSKSVPYPVAYETNEDLTWSKNEKNRLCVRFNGMSDYSFQIYCDQRQLKWFERFFQDQETKKQSQNQHPSALFTLRSARIFWQEHQGKGQPWQKHRLILYCSLDTRFWTAEGTEQIRQEKAAKITKTLSNLQAKDELTDSQQAFLRRQTSTLERINTPFPRPNQAAYQGQPHILVGVALGLEKPATVAVVDINTGRAIAYRSIKQLLGSNYKLLNRQRRQKQRNAHQRHKAQKQNKPNQFGDSNLGEYIDRLLAKAILSVAQQYQASSIVLPKLGDLRELVQSEVKVKAEAKIPGYLEGQEQYAKQYRVSVHQWSYGRLIDNIHQQASKIGIAIEQGQQPTRASPQNQARELAITAYQSRTQK

SEQ ID NO: 73 - protein, 561 aa, unidentified
  source 1..561: note = MG64-26 effector
QHKDFPQWVSAGEIPAQVVKNLVNQAQSGLPARWCASAQRQVQETYKAWITKRRKLQQKLQGQQTWLSVLRPDAELAKNAGLSLEEMKIRAQALLHREIHNWFQVYQQCQDVVERSIFAYLLKHRLTVPAEPENTDKLRQKRRQVEIKIEWLETQLAGRSPQGRDLTGSRYVAALNEGEQCYWENDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDPRLWTQEGTAMVQQEKAKKSQAIVKKLSERSDLTAQQKDCLQRHQSTLARLHMGYDRPQRRMYQGKSHLVVGISLDMENLVTVALVDVVKQEVITGCTMKSLLGQDYALVRRLRYEKRQNSHLRKVAQERGSKIVNHEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 74 - protein, 583 aa, unidentified
  source 1..583: note = MG64-27 effector
QVWEMMAGRYASLIATTLEQVSQHKDFPQWVSAGEIPAQVVKNLVNQAQSGLPARWCASAQRQVQETYKAWLTKRRKLQQKLQGQQTWLSVLRPDAELAKNAGLSLEEMKIRAQALLHREINNWFQVYQQCQDVVERSIFAYLLKHRLTVPTEPEDTDKLRRKRRQVEIKIERLETQLAGRSPQGRDLTGSRYVAALNEGEQCYWENDADFLAWQAEILSCPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDPRLWTQEGTAMVQQEKAKKSQAIVKKLSERSDLTAQQRDCLQRHQSTLARLHMGYDRPQRRMYQGKSHLVVGISLDMENLVTVALVDVVKQKVITGCTMKSLLGQDYALVRRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 75 - protein, 417 aa, unidentified
  source 1..417: note = MG64-28 effector
VEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEIAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 76 - protein, 488 aa, unidentified
  source 1..488: note = MG64-29 effector
MGKDILDEIASIGVIEDETEWNSIESALLKKQTLMPHPILFESSDDFIWAEPARFQIEDLQAEEEISRDTEAKELKAKNLQAEKESSNTEPRKRVCVCFKSFDEKYTFEVAGDYRHIHAVWQALKERKEYDKNADKNTSALFLVRSATLIWREYKKNDNRIVRRRKAAKKRAKREGLVASTESDSSLAPEFYDPEFPWNRYQLFLHCTIETRYLSKEGTEIDIEKQKKSTAKSIQTLEKNIAELEKKGESAKTRKDRHNRRSGTLRRLESYDNNYERSSKPLYAGQPHIVTGVALGSSGLVTTTISDAISGKILECRGLEALLGKDYELVKRRQFERQLNSRRRTQNQKRGANDQFGESNLGDTIDKRIANAVIDFAKKHQSGFIVLPDMNDYRRRKQSEIAAFAERECGGWKGIEKKFAKAQKEKIHSWSYGRLTTYITNQAEKEGILVRTRRQPIQGSSQEQGKLMAIEAYKDKPKPKKSRPKKSA

SEQ ID NO: 77 - protein, 467 aa, unidentified
  source 1..467: note = MG64-30 effector
KTAEILAQTTPQPAEARTAKSSKKKQKSKLNGQKQSFDLLFEQYQETDDELTRSAIVYLLKNGRKLPNEPEDPKQFAKRRRKAEIRVERTTNTLHRMQLPTGRDMTDQEWLKTLATAVSNVPEDDGEAASWQAILMSEAHKLPFPILYETNEDLSWFLNDGERLCVTFNGLSEHTFEIHCDRRQLHWFKRFLEDQEIKKASKNQHSCALFTLRSARIAWQEGKEKGEPWNTHRLTLACTVETRAWTAEGTEQIRQEKAAECKKVILGTKAKGNLNQRQEQFIQRREKTIVLMNNSFPRPSRPLYQGQPEIIASVSYGLDRPATLAIVDITTGKAIAYRSIRQLLGDHYKLLTRYRLRQQQNAHRRHNRQRKGASNRIQESQLGEYLDCLIAQAIVSVAQTYQASSIVLPDLGNIREVVEAEVRARAEQRIVGYLEGQQQYAKQYRASIHRWSYGRLSEKIQSQAAQA

SEQ ID NO: 78 - protein, 550 aa, unidentified
  source 1..550: note = MG64-31 effector
PLINALIRELVSHPEFESWREKGRHPSDVVAKLNRTLKTDAQFAGQPSRFFMSAEKVVNYIFKSWFKIQSRLQQKLSGKQKWLEILKSDEELVELCGQSIDVLQKKAVQVLQEVGKAIDKETSDTIPEKKRANLIRQRLFKKLSSAKQPLPQYAAIYLLKNHCKIPNEPEDVENFAQRRRKTEIQIQILQDQIESRIPKGRDLTGKAWTETLLKAVNSIPQDNQEHKQWQDRLLSMPSTAPFPILFETNEDLVWSANERDRICVRFNGLSEHTFQIYCDQRQLHWFKRFLEDQKTKRASKNQHSSALFTLRSARISWQENDRKGNPWETHYITLSCTVDVRLWSAEGTDEVRHEKAADVAKVLTRLNEKDSLTETQAGYARRLTSTLEKLSSPFERPSLPRYQGKDNIIAGISLGWDKPVTLAIWNTDTQEVLTYRSLKQLLGKDYPLYLRHRREQQKQSHERHKAQKQGKGNRFGTSNLGEHLDRLLAKAIVAIAQQYSAGSIAVPKLDSIRDTLQAEIDAKAEQRIPGYLEGQKRYSRDYKRSIHRWS

SEQ ID NO: 79 - protein, 446 aa, unidentified
  source 1..446: note = MG64-32 effector
SIFAYLLKHRLTVPTESEDTDELRRKRRQVEIKIERLETQLAGRSPQGRDLTGSRYATALNDGEQCDWEDDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLELISQQAIVAGLLVEQGLQGSGGTAIKRALGVALSAYYQRSAA

SEQ ID NO: 80 - protein, 649 aa, unidentified
  source 1..649: note = MG64-44 effector
MSQITIQCNLVASEATRQYLWHLMADIYTPFINEMLATIAQHPNFEEWSQNGRIPADVFEDIRKTLKAHPDFQGMPGRWYYAGRDLVKRIFKSWLALRRRLRHQLSGQTHWLEIFQSDDDLVAACGQDLPAIRAEAASILTKIQIEAPNTSKQPKKTKQPKKAGSKTQKPEEEQRNRNLFPALFKEYDGAETELVKCAIACLLKNNCQIPTKAEHPEKFQKRRRKTEIRVERIIEQLARTRLPKGRDLTNEKWLDTLKMAVQQVPKDETEAAAWEADLQTDSSPLPFPIAYESNEDLKWSQNAKGRLCVRFNGLGKHTFEIYCDTRQLHWFKRFLDDQTIKKQGGNSHSAGALTLRSGRISWRLDSSKGNPWDRNRLVLFCSVDTLLWTKEGTEKASQEKASKIAQVISGTKAKGNLTSKQEDFVRKREKTLALLQNPFPRPSRPLYQGSPAILAGVSFGLDKPATLAIVDVTTGKAIAYRSIRQLLGDDHKLLNRQRQRQRQKAQRRRSNQLKFASNRISEGGLGGQIDSLIAKAIVQIAQQYNASSIVLGDLANIREIIESEIQAKAEQKTTLKEIQAKYARDYRASIHRWSYKRLAQKIESNALQAGLIVATIKQPLAGSPQDKARDVAIAGFQSRSVSKILDTGS

SEQ ID NO: 81 - protein, 429 aa, unidentified
  source 1..429: note = MG64-45 effector
MPPCRPPATRQYLWGLAAEKNTHLINALIQEVVSHEDFEDWRLKGRHPADVVAKACRRLKTEVPFSGQPSRFYASAEKAVNYIFKSWFTLQSRLQRQISGKQAWLDMLKSDDELVELCGRPLEGIQATAAQLLKQIEKTLESEATEESQGKSKEDQVRTHLFKKYHSAKQALNLCAAAYLLKNNSKIPDKPEDPNKFAQRQRKAEIQVQRLQDQIEGRIPKGRDLTGQAWLSTLLTVTTTVPRDNREHKQWQDRLLAQPRTIPFPILFETNEDLVWSRNQADRLCVRFNGLSEHTFQIYCDQRQLPWFQRFLEDQQTKRASKNQHSSALFALRSARISWQENDRKGQPWDAHSLTLSCTIDTRLWSAEGTDEVRQEKAADTAKVLTRLNEKGSLSDTQAGYAKRLTSTLNRLDSPFKRPSQPRYQGKPH

SEQ ID NO: 82 - protein, 477 aa, unidentified
  source 1..477: note = MG64-46 effector
EQQNRNLFPALFKEYDGAETELVKCAIACLLKNNCQIPTKAEHPEKFQKRRRKTEIRVERIIEQLARTRLPKGRDLTNEKWLDTLKMAVQQVPKDETEAAAWEADLQTDSSPLPFPIAYESNEDLKWSQNAKGRLCVRFNGLGKHTFEIYCDTRQLHWFKRFLDDQTIKKQGGNSHSAGALTLRSGRISWRLDSSKGNPWDRNRLVLFCSVDTLLWTKEGTEKASQEKASKIAQVISGTKAKGNLTSKQEDFVRKREKTLALLQNPFPRPSRPLYQGSPAILAGVSFGLDKPATLAIVDVTTGKAIAYRSIRQLLGDDHKLLNRQRQRQRQKAQRRRSNQLKFASNRISEGGLGGQIDSLIAKAIVQIAQQYNASSIVLGDLANIREIIESEIQAKAEQKTTLKEIQAKYARDYRASIHRWSYKRLAQKIESNALQAGLIVATIKQPLAGSPQDKARDVAIAGFQSRSVSKILDTGS

SEQ ID NO: 83 - protein, 439 aa, unidentified
  source 1..439: note = MG64-47 effector
LKNGGKISNKEEDPEKFAKRRRKLEIQIERLREQLEARIPKGRDLTDAKWLETLLLATNKAPENEEEAKFWQDSLLKKSSTVPFPVAYETKEDMTWFKNERGRICVKFNGLSEHSFQVYCDTRHLHWFQRFLKDQQIKHNSKNQHSSSLFTLRSGRIVWQEGESKGDPWKVNHLILYCSVDTRLWTAEGTNLVREEKAEEIAKTITQTKVKGELNDKQLAHIKRKNSSLARINNPFPRPSKPLYKGQPHILVGVSLGLEKPTTLAIVDGTTSKVITYRSIKQLLGDNYKLLNRQRQQKHLLSHQRQIAQTLASPNQLGESELGQYVDRLLAKEIVAIAQTYKAGSIVLPKLGDMREQLQSEIQAKAEQKSDLLEVQQKYAKQYRVSVYQWSYGRLIANIRSSAAKAGIVIEESKQPIRGSPKEKAKELAIVAYNSRQTT

SEQ ID NO: 84 - protein, 641 aa, unidentified
  source 1..641: note = MG64-48 effector
MSVITIQCRLVAKEFSLRYLWELMAQKNTPLVNELLQAIVQHSDFESWLQKGSIPQATIKALCDSLKNDPRFSGQPGCFYTSATSLVSYNYKSWFALQKRRQQKIEGKERWLNLLKSERELEEECDRTLEEIRAKATQILTQFQPQAENAETPPGEATQKKKGKKSKSTPSKGKAASLFNTLFNAYAQTKDPLERCALAYLLKNNGEVSLVEEDLEAYQQRRRKKEIEIERLKEQLKSRHPKGRDLTAEKWLETLEAARTQQPESEAEAATWQANLLRNSSPVPFPVNYETNEDLTWFKNEAGRICVFFSGLREHHFEVWCDNRQLHWFERFFQDQQIKRQSKNQHSSSLFTLRSGRIGWQECTGEGEAWNIHHLKLYCTLDTRLWTAEGTEQVAQEKAAEISNILTKTKQKDDLNEKQLAFIKRGETMLARINTPFPRPSKPLYQGQSEIVVGVSLGLEKPATVAVIDVPGDRVLAYRNVKQLLGDHYPLLNRRRQQQRSLSQKRQKAQKQGAANEFGESELGQYIDRLLAKEIVAIARSYQAGSIALPKLTDIREILHSEMQTRVEKKIPGYKEAQDKYAKQYRVNVHRWSYGRLMDAIASQAAKVGIVIEVGKHPSEGSFQDKAREVAISAYCDRSVN

SEQ ID NO: 85 - protein, 709 aa, unidentified
  source 1..709: note = MG64-49 effector
MSIITIRCRLVAGIKQQIDKKSIKNFSAEDRALLQKLLDDKSQTDRDNPKDKQKIFLAQSSEDVRQNLWQLFFTSSALIDQLLDRLSQHPNVQTWQQQGKLPDDELKACWLELKTSPLYDEKLPGRFFSSVQSMVKTIYASWLALHQQKQRRLNGLNRLTEIVYSDETLLEMCDSTFAQLQLNAASMLSEIDKEVVNSEKSRSRIDLLFKKYTELPVEDTIGRSAISYLIKHGCKIESKIELTPKFARWFKTKLKQAQRLEHQLAGHFPRGRDLNGIAFLNALETATKDEPQDNPELMLWQSQILRDPSSLPHPIEFNSNTDLRWLKLYRKQYQCKRTVSGDSIDSIELTQRLFVEFKGLTQGSNYVFEVYCDRRQLAIFQQFFDDDRLLRNSSSEEKYSSSLFTLRSAYLLWDRQESQDRYRRHTLSIQTATEPWNTNQLYLHCSIETKSLTAEGMREIQQQKTQKVNNILAKQSKNAYPSIDQQQSQRKNQTSLNLLDRSLPRPSRPIYQGNPQIIVGLIFDPVRPIYLAVVDVTTGKPITYRSTRQLLGDKYQKLSEYRLKQQQNSHHRRKHNQQGQFQQPTESTQGEYLNRLLAKAVIQIAQEFKAASIALPPINHNIEKVQSKIEADAEEDLPEDVGTQKKITRKTSVVIHKWSYHSLLEYVKSNAAKLGITVETVSLPSLGTPSQQAAEVAISAYNSRKHIKK

SEQ ID NO: 86 - protein, 642 aa, unidentified
  source 1..642: note = MG64-50 effector
MRYAISMSQITIQCQLFADEAARKYLWQLMTARNTPLVNELLRLIALHPDFLKWRSSGKLPTTEVTKLAKTLKTDERFNNQPAKFHISAEKTAIYTFKSWLAIQKRAQQQLEGKISWLRMLRTNEELVADCGQDLAQIRTKAQALILQYQSSEDSAESQKNFRKSLYQAYDIAEPRLGAQNERSLTRSAIAYLLKNRCHIPSDTDEDLKKFLKYRRKIENQVKRLTQQLENRLPQGRDLTGERFLKTLHSATKSVSVDNTEASRWQSQLLERPDLVPFPITLESNMDLMWFITQQDKIGLHIGGISEHEFTIGCGQRQLHYFQRFLSDYQTMLASKRQHTSSLFLLRSAKSIWIPSKDKGEPWNVHQLYLSCTLDTRLLTSEGTELVKQEVAAGTTKKLVTMQEKLDRTDNQDNYVKRLQSTLDRLDRPFNRPSKPLYQGQSNIIVAVSMGLQSPVTAIAIDITTQKILAYRHTKQLLGDDYRLVNRQRNLQTQQRHSSHKAQKQGLSRQCSNSELGEHLDRLFAKAIVELAQTYQAGSIALPKLDQIRLSIQSEIDTKAQQKIPGYVEGQKKYAKQIRINLHNWSYNRVSQLIINKAGQSGIAIEYGDQPARASPNVREASRNENRAGEIALSAYAKRTIP

SEQ ID NO: 87 - protein, 467 aa, unidentified
  source 1..467: note = MG64-51 effector
MFPSFCSPPFLSYFSTPLNGYNRCAISYLLKNGCKINDKEENPEKFAQRRRKLEIQIQRLTEKLAARIPKGRDLTDTQWLEALFTATQTVPENETEAKFWQNSLLKKSSSVPFPVAYETNEDLVWVKNQFGRICIKFSGLSEHTFQIYCDSRQLHWFQRLLEDQKIKKNSKNQHSSALFTLRSGRISWQEGEDKGDPWNVHHLTLYCSVDTRLWTEEGTNLVKEEKAEEIAKTITQTQAKGDLNDNQQAHIKRKNSSLARINNPFPRPSQPLYQGQSHILVGVSLGLENPATIAIVDGTTSKVLTYRNIKQILGDNYKLLNKQRQQKHLLSHQRHIAQRMAASNEFGDSELGEYIDRLLAKEIIAIAQTYKAGSIVLPKLGDMREQVQSEIKAKAEQKSDLVEVQQKYAKQYRVSVHQWSYGRLIANIQCQANKAGIVIEEAKQPIRGSPQEKAKELAINAYQSRKA

SEQ ID NO: 88 - protein, 814 aa, unidentified
  source 1..814: note = MG64-52 effector
MAIRTMETDVIVRTSKPRESKRSIDDLVLFDEAAWCRVCEFGYQHTLFVDTIMKQVKQLPINWLYTNQELQKQDDTTHETETKQGQNGLPEGLVKALHDALASTPQLSKMSGRFYASAIDRVEEIIESWFAAHQKLIHKLRGQRRWLAAVESDTALAESSHFSQYEIEQRAAQILAELEAKGELTAASGNQTPFNVLFKQFDETEAVLSRRAIIHLLKNINEVQVKLKQPKKGKGRNKAAQRSLTERLEAKRVEIERLEKQLLGQLPRARNLFPDQAFDQRLEALIDMPLSEATELERYYFLYFSLLLYLSGSNTYVQLEQHLLIALVLQWSKLDDLHYYRALNYSFILYAASEQQYLQLGSYLLQSIKIEAERVEAAFFAWHESITPKLQDFLREPKALPYPISFGYEDVRSWHVNQKGKIFFKLNGWGDLLFEVRCHRRQLSLIKTFCKDWQTKNNAEHKGQYSGSLMLLRSIELIWKPKKASEQKDAQLCSQCEVFQQYGGKGFWNECKLTIHWTFDTDGLTKQGSEQIRQRKLKPQLKKLQDDQAALEQKQDRLNKLEQQVAEPSQSQAQLKRLEPLKKEVQKLQEKLAKPRPKLDCLQAAKPFDRPDRPLYEGIPNIFVGVLLDLDKHLVVTVVDAMRRKRLTLRSARKVSPEGYRLLQKYFRQRQEHSQQRQQDQKAQRRSHQTESGLGQQVARLFAKGLVELAQAYKASTIVIPITGGWRERLYSQLVARAKIRCNGSKQAVDRYTKEHGERLHQWDYNRLSQAITDCAATHGIEIVLQKTVFEADVFQQSANLAIAAYDSLNSEPP

SEQ ID NO: 89 - protein, 637 aa, unidentified
  source 1..637: note = MG64-53 effector
SMSQITIQCQLFADEAARKYLWQLMTDRNTPLVNELLRLIALHPDFLKWRSSGKLPTTEVTKLAKTLKTDERFNNQPAKFHISAEKTAIYTFKSWLAIQKRAQQQLEGKISWLRMLRTNEELVADCGQDLAQIRTKAQALILQYQSSEDSAESQKNFRKSLYQAYDIAEPRLGAQNERSLTRSAIAYLLKNRCHIPSDTDEDLKKFLKYRRKIENQVKRLTQQLENRLPQGRDLTGKRFLKTLDSATKSVPVDNTEASRWQSQLLERPDLVPFPITLESNMDLMWFIAQQDKIGLHIGGISEHEFTIGCGQRQLHYFQRFLSDYQTMLASKRQHTSSLFLLRSAKLIWIPSKDKGEPWNVHQLYLSCTLDTRLLTSEGTELVKQEVAAGTTKKLVTMQEKLDRTDTQDNYVKRLQSTLDRLDRPFNRPSKPSYQGQSNIIVAVSMGLQSPVTAIAIDITTQKILAYRNTKQLLGDDYRLVNRQRNLQTQQRHLSHKAQKQGLSRQCSNSELGEHLDRLFAKAIVELAQTYQAGSIALPKLDQIRLSIQSEIDTKAQQKIPGYVEGQKKYAKQIRINLHNWSYNRVSQLIINKAGQSGIAIEYGDQPARASPNVREASRNENRAREIAISAYAKRTIP

SEQ ID NO: 90 - DNA, 376 bp, synthetic construct
  source 1..376: mol_type = other DNA; note = MG64-2 effector intergenic region encoding tracrRNA
cacacaacagaaccttgataatagaatagtaaataattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactttaattttttttcgtaaagccaagcgggagccaaaaccctagggggtttacgaaagtctcacgattcttacattgagtaagtttcagtgtttttgggatggttaaccctttatttacaag

SEQ ID NO: 91 - DNA, 390 bp, synthetic construct
  source 1..390: mol_type = other DNA; note = MG64-4 effector intergenic region encoding tracrRNA
tatacaaacgtttgaacctggaaaatagaataatgagaaatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttaattattttttggcaaaccaaagtgggagcaatttcactgggaggttcgccaagttttcaaacaacttattttgtagaggttttgccgttttctgctaagaaaagattttcttgtccagatgcagtgataa

SEQ ID NO: 92 - DNA, 462 bp, synthetic construct
  source 1..462: mol_type = other DNA; note = MG64-6 effector intergenic region encoding tracrRNA
gatttatcagtaccgtacctggaaaattgaatataattgataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccttaattttttattttttcgtgaacctaagcgagagcaaaatctctaggaggttcacgaaaaagctgaatccttcatggaatatgagtttcagtttgttagtgggatgatggcttctcctgaaaaggagaaatggagtaagaattttagaggtttacgaaaatgacctttaaaagctacttcaggcaagtgttacagcgctcgc

SEQ ID NO: 93 - DNA, 456 bp, synthetic construct
  source 1..456: mol_type = other DNA; note = MG64-7 effector intergenic region encoding tracrRNA
tcaaaaagaccgtaccttgaaaatataatggtaacaaacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttaattatttttttggcatggcaaagcgggagcgattttaccgggactgatgccaaagcttcaaatctttttattgacaaggtttctagacttttgtttgtcacttgatttatttttttaattgtcaactagcaagtgattttggtagttttgccaaaagtgtctctaggaatcttgataaataaagggttttaggcgcg

SEQ ID NO: 94 - DNA, 37 bp, synthetic construct
  source 1..37: mol_type = other DNA; note = MG64-2 effector target CRISPR repeat
gtttcaacgaccatcccaactaggggtgggttgaaag

SEQ ID NO: 95 - DNA, 37 bp, synthetic construct
  source 1..37: mol_type = other DNA; note = MG64-4 effector target CRISPR repeat
gtttcaactttccttccagctagaggcgggttgaaag

SEQ ID NO: 96 - DNA, 37 bp, synthetic construct
  source 1..37: mol_type = other DNA; note = MG64-6 effector target CRISPR repeat
gtttcaaccaccatctcaactagggatgggttgaaag

SEQ ID NO: 97 - DNA, 38 bp, synthetic construct
  source 1..38: mol_type = other DNA; note = MG64-7 effector target CRISPR repeat
gtttcaacgccccttcaagctttgggcgggttgaaagc

SEQ ID NO: 98 - DNA, 37 bp, synthetic construct
  source 1..37: mol_type = other DNA; note = MG108-1 effector target CRISPR repeat
gttgcgatcgccgctccggtggcgatggggttgaaag

SEQ ID NO: 99 - DNA, 28 bp, synthetic construct
  source 1..28: mol_type = other DNA; note = MG110-1-6 effector target CRISPR repeat
gtgacctgccgcataggcagctggtaaa

SEQ ID NO: 100 - DNA, 27 bp, synthetic construct
  source 1..27: mol_type = other DNA; note = MG110-2-6 effector target CRISPR repeat
tatgaactgccgcataggcagccaaga

SEQ ID NO: 101 - protein, 922 aa, unidentified
  source 1..922: note = MG64-13-B transposition protein
MLSSQAFNDWCQRLQINSVARAVIEQVRNAAPSRQVQGRRKNVCGSYPSRKMGVTIQFESHRNELARIHELEHDQTVLEYYDQPPPIELVYSSKTGRRTRHQYTPDFFVLRTDGVEWEECKTETELIKLAQENSNRYCQDVEGKWHCPPGEAYAQVFGFQFRVWCNSEINWVLQDNWVWLEDYLGVEVPSVSPAALQALQRHLQQTPGICVSELLAVEPTVSPDHLYHLIATEQVYVELAAARLSEPEQVRVFLDQDMAQAFHRIQGEFELVQTNKQSMLNFSIGTFLHWDEVEWEVINIGANTVGLLRSEGEVIELAKPTLENLVSRQRIVASYNSENIEKQKIINDKIKAILQEATSAEIAEANRRYDLIQACSNTSRHPSSTVRRWQNQYQKAEKIYGNGYVGLLPRYHTKGNRTMRIDDIAYQFMIEFIEQHYENPKQRRKQRVYEAFVSACEAHEPKLIPPSRITFSQAIDKRGGQAQTLKREGRRAAIAKEAFYWQLERTTPQHGSRPFEIVHLDHTQLDIELMSSLSTLAVQPLASNHRTLSQNLGRPWATFMVDAYSRRLLAVYLTYEEPSYRSCMMALRICVQRFERLPQILVVDNGVEFHSHYFEELLAYYACTKKHRPPAMARFGSVVERLFGTANTQFVHELQGNTQITRQVRQVTQSINPKRLAIWTLSDLYPALCEWAYEIYDQREHPALGQSPHDIFTQGLALGGTRDHRRVENNEVFQILTLPAPDQRRRKVQAGRGVKIHNIYYWSNAFRDPKVEGSQVEVKYDPFDASIAYAFVHHQWVKCISTYCADLQGRSERELRLVSEELRQRQRVAGCRHSLSDKELVEFLNSQHAKEGQFLQRRLQAVEQLAVLQQLDPPVDDADAESQQIEFGGDAQPSCNGNSPQTAQTVPAIVNFAVPPEFYGEF

SEQ ID NO: 102 - protein, 364 aa, unidentified
  source 1..364: note = MG64-13-C transposition protein
MAVQDGFPYELLDQPITDRVAYFRHYTMAHPVLLNAAQILMQTVREPAGISLVFVFGPTGVGKSTLLRRVSQKLTEAMFAALQQDQGRIPMTGIEAATPEFGQFDWKDFYLRALAAVQEPFIYPWSTSRLTTLKLRLALEHALRKRQLQVFYIDEAQNFAKVASARKLSDQTDAIKSLANLTGVQFVLTGTYEVLMLRNLSAQLCRRSIDIHFPRYRAEIPEDLQSFRGIVQTFQRHLPLPQEPNLLEQWEFCYERSLGCVGILKDWLSRALVATLEAGAKTLTPQMLEQHAWSLERCMIMLAEAKEEEKKLEVQPLQSALRIALGLETAVVEPASPPTPSVSPRRTAISAKPKRYPVGGGQHD

SEQ ID NO: 103 - protein, 267 aa, unidentified
  source 1..267: note = MG64-13-Q transposition protein
MIEVPSIFESWSLETFQLPRRSHLYSLSPAGLATDAIESLTSYLTRLAEAHRVSVRKLLDEKILLLLKPPRTFKYEIQSAQSWLAATPEVAKAVECLRRLTLRQDLQHLTLLPWQHQLSTPSVFHDVQPFCPVCFEEARNTDAAVYEPLLWTLEAVKVCLRHRRYLQFCCTYCGRKQPFLKLEARPGHCAHCGSWLGLELPFFVSTEPEELDWHINLAQMLGQRLQAAWQRTNQSLSKAKRSKQAQKPSLMSLLESRYELDEIKLVY

SEQ ID NO: 104 - protein, 218 aa, unidentified
  source 1..218: note = MG64-54 effector
MTIITVQCRLKAPEATRRTLWELMAGRNTPLINKLIELLRGHPDLDKWRDKGKLPAGVVGELCKTLKADPLYNGQPSNAYKSAVETVEEQFESLLALHLELKEKLRIKTRRLAIFKSNAELMQLSGLTLDDMKKRAGEVLNELKEDERKKPIGTLYRKYELLFFDDPDRWAVCYLIKNGGKLPKGKEDPEKFEKSRRKLEIMVQKLTMRLDSCLPGGR

SEQ ID NO: 105 - protein, 541 aa, unidentified
  source 1..541: note = MG64-54-B transposition protein
MDKTNNPTLTEGLEGEAALRLEVITDLLQPCDRSTYGQRLREGAAKLGISVRSLQRLFKRYQEEGLSALMSGPRRDRGAHRIGEEWIQFIVKTYQDGNKGSKRLTPKQVAIRVRAQAHQKGQESYPSYRTVLRVLEPSIENKNKSIRSPGWRGEALSLKTRDGDALEAIRSNQVWQCDHTRADVLLVDREGRLIGRPWLTTVIDSYSRCIVGIDLSFDAPSSQKVALALRHAILPKLYGAEYKLNCEWNAYGKPECLFTDGGKDFRSNHLAEIAAQLGFILKLRDRPSEGGIVERPFKTLNQSLFSTLPGYTGPNVKERPEDAEKEATLTLRELEILIVRYIVDNYNQKQDARTGDQTRAQRWEGGIDLPEPLSERELDICLLKSTRRKIQRGGHLQFENITYRGEHLGGYEGDYMNLRYDPRDITSIWVYRQESSGDVFLCRAHAVGLQTEILSLNDAQAAAKRLRDKGRGLDNNSILQESIEREATVTKNLQRRKKEEQAYKTPTPAPEEKVELTPEVSKIEDIEHLEVWDLDELNGWG

SEQ ID NO: 106 - protein, 268 aa, unidentified
  source 1..268: note = MG64-54-C transposition protein
MEDKKLPIDQEWLNGEIARLNKKTVIPIEQVKSLHDWLDEKRAARQSCRVVGESRTGKTVTCQSYALRNTPRLSGNTPPNLPVVYVVPAQKCGPKDFFGAIIGALIYRAVKGSTSELRNRATEILKSCGVEMLIIDEADRLRPETFSEVRDIYDNLEIAVVLVGTDRLDAAIKRDEQVYNRFSACRRFGLLTGEDFIRAVAIWEDKILKLPVASNLTSKETLKILTRGTGGYIGKLDQILKGAAIRSLRKGHKRVETEVLKEVVKEFS

SEQ ID NO: 107 - protein, 178 aa, unidentified
  source 1..178: note = MG64-54-Q transposition protein
MSEDIKPWLLPVQPEPGESLSSFLGRVRQRNHLTAGALGNITRLGATIARWEKFYLNPFPSAAQLEAMAAIVGLTGEFIRDMLPPTGEAMKCEPIRLCPECYAESPYHRLQWQFQSVWKCTSHGNKLLSKCPVCSASFGIPSNWNGYCRRCGTPFELMRARSESIPALSPVDDKGYDR

SEQ ID NO: 108 - protein, 638 aa, unidentified
  source 1..638: note = MG108-2 effector
MSQITIQCRLVASESTRRELWELMAEKNTPLINELLEQIGHHPDFETWREKGKLPADVVKQLAEPLKTDPRFIGQPGRFYTSAIALVKYIYKSWFAVMMQLQYQLKGKIRWLEMLKSDEELVETSGITLDNLRIKAAEILAQCTPQPDSVESQEKKSKKRKKGKKTKKSDSQPSLSKNLFDTYDSTEDIISKSAIVYLLKNGCKLTDKEEDPKEFAKRRRTTEIQIQRLTEQLSARIPKGRDLTNAKWLETLAIATENAPENETEAKSWQNKLLRKSASVPFPVSYETNEDLTWFKNNKGRLCVYFNGLSEHIFQIYCDSQHLHWFQRFLADQEVKENSKNQHSASLFTLRAGKIVWQEGEGKGEPWNINHLTLHCSVDTRLWTAEGTQQVRKEKEVEIIKFLNKNKDRSDLSKNQKANIKRQNSTLVKIANPFPRPSQPLYQGQGQILVGVSIGLESPATLAVVDAITKKVITYRSIRQLLGENYRLLNRQRRQKQSLSHQRQKAQRLADGNQLGESELGQYIDRLIAKKIVVIAAAYNAGSIVLPKLGNVRQIIQSEMDALAEQKCPEYKEGQKKYAKQYRINIHQWSYGRLIECVKTQAAKIGIVIEEAKQSIRGSPQEKAKELALAAYDSRSND

SEQ ID NO: 109 - protein, 572 aa, unidentified
  source 1..572: note = MG108-2-B transposition protein
MYMQDDRTRSAEIPAPSSGEGNSEVETSEVITELPLEARLKQEIIQSLLEPCDRITYGQRQREAAEKLGVSVRTVRRLVKKWEEKGLEALTQTARTDKGKHRIDEEWQEFIIKTYKEGNKGSKRITPKQVAVRVQVRAAQLGEEKYPSYRTVYRVLEPIIEQQEQTQNIRSRGWRGSRLSVKTRTGQDLSVEYSNHVWQCDHTRVDVLLVDRHSKILGRPWLTTVIDTYSRCIVGINLGFDAPSSQVVALALRHAILPKQYGLEYKLHCEWGTFGKPEHFYTDGGKDFRSNHLQQIGVQLGFVCHLRDRPSEGGIVERPFGTFNTELFSTLPGYTGSNVQERPEEAEKEACLTLRELEQLLVRYIVDKHNQSIDARMGDQSRFQRWEAGLIAVPSLMGERELDICLMKQTRRTIYRGGYLQFENLTYRGEHLAGYAGESVVLRYDPRDITTILVYRSEEGKEVFLARAYAQDLETEQLALDEAKASSRKIREAGKTVSNRSILAEVQERETFLTQKKTKKERQKEEQVQLRKAKQPLPIEPEEIEVASVRDEAESEMPEVFDYDQMREDYGW

SEQ ID NO: 110 - protein, 240 aa, unidentified
  source 1..240: note = MG108-2-C transposition protein
MTTQEAKTVAQQLGDFPVNDTKLQAEIQRLNRKTFVPLEQVENLHEWLEEKRQARQSGRVVGESRTGKTMGCNAYRLRHKPKQELGKPPIVPVVYFKVTQECAAKEFFGLIIQNLKYQVVKGTVPELRARAVQVLKGCRVEMLILDEADRLKPKTFAEVQDIFDDLELAVILVGTDRLDAVIKRDEQVYNRFRACHRFGKLSGEEFKRTVEIWEKKVLQLPVASNLFSKTIERGKCSQNG

SEQ ID NO: 111 - RNA, 261 nt, synthetic construct
  source 1..261: mol_type = other RNA; note = MG64-6 active effector sgRNA
ataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccgaaaggatgggttgaaagnnnnnnnnnnnnnnnnnnnnnnn

SEQ ID NO: 112 - RNA, 263 nt, synthetic construct
  source 1..263: mol_type = other RNA; note = MG64-2 effector sgRNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaagnnnnnnnnnnnnnnnnnnnnnnn

SEQ ID NO: 113 - RNA, 265 nt, synthetic construct
  source 1..265: mol_type = other RNA; note = MG64-4 effector sgRNA
gaaatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttgaaagaggcgggttgaaagnnnnnnnnnnnnnnnnnnnnnnn

SEQ ID NO: 114 - RNA, 276 nt, synthetic construct
  source 1..276: mol_type = other RNA; note = MG64-7 effector sgRNA
gtaacaaacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttgaaaaagctttgggcgggttgaaagnnnnnnnnnnnnnnnnnnnnnnn

SEQ ID NO: 115 - RNA, 246 nt, synthetic construct
  source 1..246: mol_type = other RNA; note = MG108-1 effector sgRNA 1
gatgctaagtcgcgcctagcatcaaggagctatgtcttgattgtcttgggtgtccgccctggatgagttgaggtgtagatgcttctatcatggcagctactaaacgccccaagcaaggggaacccatctttaattttggcaaaccgaagcgggggcaaaatctccaggaggttcgccaaaacctttgaaactccttagctggaaatggcgatggggttgaaagnnnnnnnnnnnnnnnnnnnnnnn

SEQ ID NO: 116 - RNA, 274 nt, synthetic construct
  source 1..274: mol_type = other RNA; note = MG108-1 effector sgRNA 2
cttgttcgagttcgtttgaactgaacaagggtaagtatgggccagtttaattgctttccgtcccaggatagctgccagcttctaccgtaggttcgtcctgcaagtgatgctaagtcgcgcctagcatcaaggagctatgtcttgattgtcttgggtgtccgccctggatgagttgaggtgtagatgcttctatcatggcagctactaaacgccccaagcaaggggaacccatcgaaagatggggttgaaagnnnnnnnnnnnnnnnnnnnnnnn

SEQ ID NO: 117 - DNA, 550 bp, synthetic construct
  source 1..550: mol_type = other DNA; note = MG64-13 effector intergenic region encoding tracrRNA
gttgaggtggatttgtgtactgggtcaaaagatttcgctaggtttgccttttcgcgcgctgctgaagcagtgaaaagttccgacagtttaaaagttctaacacttttactgctttccgtcgggagtagttgtccgcttctgcgttgttgagaccgaagtttgtccaggtgataggcagaacgttgatgcaacaggtagatagtcgcgctctatcaggaagctgttctagttattagtgttcgtgcactaataaagaggatacagggatacatgtggttgtgtccagcaatggcacaacagggccactactcgaagccccgagcaagggttgagcctacccaaattttgtactaaaaatttggcaaaccgaagcgaggtcagtttgacccagcaccttgtcaatcagtcgaaatcttgcaccagtctgcgttttgagttggcaacagcctcacgctgaaccacttcaagcagaaagttatggcaatgtgatgacctttgccaaaataggtgtgcaaagccgactgtagcaagaatttgagcactaggagtt

SEQ ID NO: 118 - DNA, 457 bp, synthetic construct
  source 1..457: mol_type = other DNA; note = MG108-2 effector intergenic region encoding tracrRNA
tactagaaaaatttgcaccttgataatagaataatagtcacaatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccttaactattttttcgtgaatccaagcgggagcaaaattcccaagggatagacgaatttggtgaaataaatagccagacttaagtttgagctttttctgggatggtcaaccctcctgttacgacagtttccaaagtaaaattttcgagttttacgagttttgtttctggaagctagtacacaaaagggttgaagcgtttaaa

SEQ ID NO: 119 - DNA, 36 bp, synthetic construct
  source 1..36: mol_type = other DNA; note = MG64-13 effector target CRISPR repeat
tggcaattgcccttccagtgttgggtgggttgaaag

SEQ ID NO: 120 - DNA, 37 bp, synthetic construct
  source 1..37: mol_type = other DNA; note = MG108-2 effector target CRISPR repeat
gtttcaacgaccatcccgacaaggggtgggttgaaag

SEQ ID NO: 121 - RNA, 60 nt, synthetic construct
  source 1..60: mol_type = other RNA; note = MG110-1-6 effector crRNA
ctggtaaannnnnnnnnnnnnnnnnnnnnnnnnnnnnnnngtgacctgccgcataggcag

SEQ ID NO: 122 - RNA, 59 nt, synthetic construct
  source 1..59: mol_type = other RNA; note = MG110-2-6 effector crRNA
ccaagannnnnnnnnnnnnnnnnnnnnnnnnnnnnnnntatgaactgccgcataggcag

SEQ ID NO: 123 - DNA, 385 bp, synthetic construct
  source 1..385: mol_type = other DNA; note = MG64-6 active transposon end LE
gggaggaaaggttaattggggggttgatttattttggttggttgtgttatatttgttattcgcgggcgattagcacagtggtagcgcacttccttcacacggaaggggtcacaggttcaaatcctgtatcgcccatttgtgtacagtgacacattaattgtcatcaatgacagattgctgtcgtggagccaaattatgtgtcgctgagacaaattaatgtcgtttaactatcagtgacaaatttttgtcgcttttcacaacaatagtgtgaaggaagtgcgcctttcaatccatcctagaaattataattccaatccctacttacctagaatggtggttgaaactgtaagattcgcgccgctaataaaactttcagcgatttgga

SEQ ID NO: 124 - DNA, 313 bp, synthetic construct
  source 1..313: mol_type = other DNA; note = MG64-6 active transposon end RE
aacacataatgctatcctagcttacaaaaaagaaagcgacaatcaatctgtcactcctcaaattctcttctttgagaacgacgacagctaaattgtcactgattgggaggacgacacttaatttgtcactaacggctagcgatctttaatcaaggaaaacagctagaatatagagaaatcaatagttttcatcagcgacaacaatttggcatcacgtcaaataattagtcactgtacaggttttatttttaatgtacagtagccctcttgcaaaagtccaagaattaggaatgttagaggaggtttgataact

SEQ ID NO: 125 - DNA, 300 bp, synthetic construct
  source 1..300: mol_type = other DNA; note = MG64-2 putative transposon end LE
ttgtaaaacctcagatgagcaaggtattgatgaataatattttccgccaaaatttacctctgtttgggcaataattgtatgatagaatttatatattagttgcttcaatagcaaaatgcgggtgtagttcagtggtagaacgtcaccttcccaaggtgaatgtcgtgggttcgagtcccatctcccgcttgtgttgttgttgtacagtgttgtcgattgccaaattatttgtccgcgttgacaaattgatgtccgtttgccaaattatttgtcatttacaaaactcgaacccacgacatt

SEQ ID NO: 126 - DNA, 400 bp, synthetic construct
  source 1..400: mol_type = other DNA; note = MG64-2 putative transposon end RE
atctacaaaattgttttatacaactatgtttgtattttagttctcaggacaaagattgtgtcaattcttcacaaaagttgtaattctcgcctttttcaaaaaaggacaaagaatttgtcaactttcctaaaccggacaactaatttgtcaaaaagctttttcaatatataaagggggtataatagctgtaacccttgcgtagagtaggttagtgagctactaggtttggcggtcaataatttggcaagtggtcaaataatttggcaatcgacaattatttatcgttggggacgaatcagtgtcacggagtcaaattatgtgtcgttgtgacagattgatgtcgtctaagcaccagtgacaaattgatgttgctttaataatttaaaatcgtcacgaaata

SEQ ID NO: 127 - DNA, 489 bp, synthetic construct
  source 1..489: mol_type = other DNA; note = MG64-4 putative transposon end LE
tttcgggttctccagttgtgctcacaaggctgcccgtttggcttcatcccgttggcaatacccatacgtttttttttacggctgtgtccaatcttccgtagtgccctcgaaatcgtgcgctgactaatctccccctcccatagctgtgccatctcgctttgggttttatccccatgctctttgacgaaagcccgaaaattctcccaatcgctgattttgcctgtctgttggcatgcttgccttggctttggcttgacatcgcccgtttcggcttggcgctcaaaccataagttaatcgtattgcgactgatattgaacagttgactcgcctcacttttcttcagaccgtccaactcaatcgcttgcattactttttgccggaagtcatcactgtaaggtttagccattgaaatgaggtagacgcaatgggtattcttctagcttacgtcctaacctgcatggctatagctatactatccacgacttgga

SEQ ID NO: 128 - DNA, 602 bp, synthetic construct
  source 1..602: mol_type = other DNA; note = MG64-4 putative transposon end RE
aagtgttcaatcaaaacacatatgtattataaagttaaaaggacatttattttgtcaatgacagtaaaaagtcacaaaaatattgatttcagaaaaaagacatatattttgtcgaatttacaatataggacggctaattcgtcaataacttcgtgctaggaattagcgatagaaaaacaataaaacccttgcatagcaaaggctttagagtaaataataagttgtcaattaatttggcaaacggtcagataatgtgacactcgacagaaatcctttctagggaaaggttttgttgtttatatatctaactttgttcgagcgctcattctcaataagcagcctagaaatgggtacagatcgagatatctagagccggaattgagataggattgagataatttaatcagggccattaagcatatggcaagggtttcggacgctacgaagaaaccacatgagataaacccaagctctcaatctttatagctcgtaagctcaacaatttcttaagtcacattgaaagcactggttctaacgatttcaatctctataggcagtcagaaaagaatccaagtgaagataaacctttgtttgtgctagtg

SEQ ID NO: 129 - DNA, 681 bp, synthetic construct
  source 1..681: mol_type = other DNA; note = MG64-7 putative transposon end LE
agttaggagtttgaagttaggagtttgaagttaccactcagcactcaatactcagcactttctactcatttccctttctttgtggtacactgcaatataggctagtcaaactatgccgggatgtagcgcagcttggtagcgcacttcgttcgggacgaaggggtcgctggttcgaatccagtcatcccgataattatttagttatttatacgacatattacacgtaatgaaagctgtacattaactaattatttgtcactgttaacagattgatgtcaaatttcacaaattgatgtcactattaacagattagtgtgcttgagctagccgttaagactcagcttttgattgtagctgaaatttatttgtttgctatgttacaggctcacccttgaaattaattataactggacatgactttacttgttcaaatacgaactgttctggtatataatgatttttgtttggggtcatacttcaaactgttaggattattttgcaatatacatttcggcagtttttgacccttcctaaaatcttgtgagaaatccgggacaactggattcgctttcaacccaccactaataattttaaattattctttccactgttgattaatccatttaatttcagctaaggcttaaaacatcaaaatattcaaagtatctcactgccaacaga

SEQ ID NO: 130 - DNA, 415 bp, synthetic construct
  source 1..415: mol_type = other DNA; note = MG64-7 putative transposon end RE
atttgtgttttataaaaacatagttgtattatactgacacctggacaaagaatttgttaaattttgcaaaatcattggtttgtttgttttttggaaaatgacaattaatgtgttatatttctgagattggacagttaatatgttaattaaggtttgaaaggctttaaatctgaaaataataaaagccccatcagaaacctttctatgacagggctttcaatatttgatagagttgtgacaataatttgttaagttgacaaataattagttaatgtacaaaatatttaatgtatagttaaaatattgtaatagtaacggttttgtctatgaattatagcaaaggacgggcaaggatgcccgttccacaagattttgatttgtagtttactttggaatttttctttttattatttaa

SEQ ID NO: 131 - DNA, 260 bp, synthetic construct
  source 1..260: mol_type = other DNA; note = MG64-13 putative transposon end LE
tgtcactgtatccataaggtgagccattgtgaaagcataagttgagccaaaaatgcttgaaatgctgatttttgcgttgcttgatttagtttaatccaaacggtttgaaagcataacgtgagccaaagttttattttgaaagcataagctgagcaaacatttgcggcaacggcttgaacagcctcaaaattgtgattagctgaaatcagaagcgatgagttccggtttcacggttgtaactcttcaggaggctgcgaaga

SEQ ID NO: 132 - DNA, 1979 bp, synthetic construct
  source 1..1979: mol_type = other DNA; note = MG64-13 putative transposon end RE
agagcctcactttatcgcattgccacacaagagcgcacacataaaacattaggggtaggttgaaagaggcaggggtctgcaaattaaataccaagcttgacctgaagcagcattagctttaggaactgcgatcgccatcatgcgaactgctttattaatgcagtgagcagaaattcaagtttcgacgagtaattgtcgattattagaaggagttacaaagtatggagccagaagatacggaattgagagtgagcaaaccaatccaagtgggagcaagaaatcaattctggcaaatagattgcactgtttttgatgtcagtctggtcgagaaaactactccagattattggcaaatggattttactgctcttgatgtaaatctggtcggggaaactacttcagatgaccctctttaaccctagctactgtgcgcgacacacaatcgcaaactatgttgggataccaacttacatctgaagaggtacatgggagtgcgatcgcaacactacttcatgctgccgctcgaaaaactatctatcacaatatgaattatttaatctgcgttttcatggaggcatgatggaagaacataaatttggaatcactaatgcagttatccttaattcaatatcagggtacgtcaaactcacaagaagaagacccagcaccctcaaggatattgaagagatggtgaatcgatattttcagcaaagctcaggtgactagaaacaatattcatccacgaatgcttgataagtatagcttgattctcgtctgagcgagaatcaagctggcatatatcgttagaacttcgtggacaacagcacaaaaaacaagagacaaactgctcgttaataaactcaagtttcgcattaaagctatttagaaatgggtgaagaccgttttatcttcatctatttttagtctagctcataaaccttttggaacagtacgttacaaattaattacattagcagccggggacttagcgctacagataaacgtgaatctgggataagctgaaacgcatttaacccgagataaagttagctcatctaggtatagacacactagtccaattaatttggaggctttcttattcaggttatacatactgaaactcttttcctctctatctgccaagcaccgcaactaatttgatagaaggcaaccaggaaatttattggataaattggcgcggtaatttgacgatgtgttagggcagtgatgcactgcttcttgtgaagaagcgtttaaagagtaatttgtctctcatatttcttgctcaacgcagcaaatgtgatgctagtgcaaaatagcgtgacgggtgacacttactaaattgagtcagccgagcatcggatggtgatatggagcgacagcaaaccagagcatcggttgccaatccagcaatctagctgactaagcagtgcaagtaatttgacagcagagcaaaacagcgtgactgttgacaaggagaaatttcatttctagatcgtgccattaatttgcgaatttaggtgaattttgtgctatcaatttgcgaacctagaatttgtgccattgatttaagaacaaaggatcgtgcagtagaccgccagatcggttgcaatgcactaggttcgattccttcaatgtgctttcagatgaacaagcaagtatctgtgccactgatttgcgaatcggtcgaagatttgtgccacagatttacaaatgactattcgaccctaacaagccgattgtgccagcaatttgcgaatcgtgccatattttttgattattcacacgtgcgactttcccaaaagtcagccaaaaatcgctcaaaacgacattagcgtgcgacaagcgacatttatcgtgcgaccgtacatttgcttttacaaaggcggcacccagattcgaactgggggtggaggttttgcagacctctgccttaccacttggcgatgccgccgaattgcatcatggacattgtatca

SEQ ID NO: 133 - DNA, 1444 bp, synthetic construct
  source 1..1444: mol_type = other DNA; note = MG64-54 putative transposon end LE
ccgccggagggtcgagcgacaaactgagcaaaagtagtgataaattttgtctaaaatcaagcgacctcattctctgtacagtgccagattaaatgacgtgatgccaaattagtgtcgtcggttgatacccttgctacatcaacactctacagcctcataaccattcttagacctatcactttagtgccaaattgtgagtcgtttaatcaaataatgacagattaatcgtctttaaagaattaatctcagtgacgaattaactgtcattatccgacgatagccccaagataatatcatgttgcctctaacatgaatatgtaatggggagaatatcctaaaccattagtatcctttcctaaataaacactttcattcgcgcgaagagttatagcgccgttattgccgataaactcaagcgccgttattgccgataaactcaatggtgttattctacaacggcggtcaattaatatttatagtgaaactcctccatttttagtaaaaaatagcaacgtaacaacatagggctatcgtttattataaatacgtaattcccgaaaattagtgggaaggtcacaccttccatccgttccatatatctgagagctgaattgatttgaattcatcaaactccaagtcgcatacattctagtagtacagccatttcttctatcctgacgtgtataagttacagaggcttgattataactcttatggcggcctttaatttgatgatcgaaatctcctgaatatagttcagcattaatattaactccatcatcactagaattaataactactgcgtcctgttctgaagcttgccacataccattaaatgctccaggtgctctattttgagcaaaagcagaagacaatccagaaatagctattacgcaaatagaagaagcaagtacaagtacgagttttttaggtctcatggtttttaattctaaaagctagtcaatttgtgagattgaagggtgttaaactaaagcagaggtagctacaaataatgtagaagctacgaaattacgtatgaaacttttttcaaatccagcgtggtcattatggatagtgatttttacgcctatagcttcttattctaaaggaatttcctattaacgatcgccgctattgtagcagtggcataattgtcacactaaggattgactcccaaccgtataattaaacactatttgaaaggtaggggcagattttgatgtttgctactccacggtataagttttcttcggggagagctaaaacgtcctttattcgccgatgcaggcgaaaccatagagttttgaattacagggtggaagacccaaacaaaacgtgccgctcatgttctgttgtctccgccagtgctaaatgccaaactaattgtcgcattatcttagtatgttgctaggataacaatatgttttattaaaatttttattta

SEQ ID NO: 134 - DNA, 307 bp, synthetic construct
  source 1..307: mol_type = other DNA; note = MG108-1 putative transposon end LE
tgtccattaaagaattatttgtcgcctttaaagaattaatgtccaatttcaggaattttggatgtttcaggctttggcaagaaatctcttgctcaaaattttggtttaaagaattaatgtccaaaatttcgacatacttgcgttgaaatacatcactcatcgttagagtgcgatcgcagcgctcctttcaacccatctacattcacacaaaagcactcttaccaaagactcttactctttcagtgcaatcaaccttggaagggcgattacaaagcagtcttccaaattcagcgtagtattgacattc

SEQ ID NO: 135 - DNA, 619 bp, synthetic construct
  source 1..619: mol_type = other DNA; note = MG108-1 putative transposon end RE
ggtgtgagcagttaacaaccatggcttaaataatgacactttttggtttaaatattcaaccagcgaatagtaaaatttatgacaaattttgctttaaacaaagctatggctattttggtgaagtgaatcagcctgaagatcaatagcagcaagggttgcatcacctagcagtgtttagagccaagatgacaataattctttaaagtcggtcaaacaattctttaatggacaccatgtgtgatgtacactaacgctcatctgtcactctaggcagatgtccattaaggagctatttgtcagttttaaacagctcttgtcaaaaaattttagtactcaacgtattttcaagggttttaagcgacaccccttgatttgctgcttttttgccctgaaattccagttttaaggagatgttgtcacagatgattcatgccttgctaccctgtatcggtatatggacagagtcattacagacgatcgcctgtagtgcgatcgcagcgctcctttctatccacattaattcacatgttttaacgctcaccactaatcgactgctgagcggttgctgtaatagcgtcgagacgtatgtaggccgaaatcgatcgcatccgcaacagcg

SEQ ID NO: 136 - DNA, 709 bp, synthetic construct
  source 1..709: mol_type = other DNA; note = MG110-1-6 putative transposon end LE
tattgttatccctatttattgttattagacaaattgtttatttaaacaacaataaatagataccatcaaaatacaatttgtacagtgataaaatcgaaaaaatagataacaatttgttatatgcagcgcgaaaactggaatagagaactgagcggcgtggtttaatatatgagcggcaatagtggaagtttaagggggaatgtatcgctgacgggtatagcttgttgattgaagcataaattgacaaattcacaaacgaaaaatcaatgagttacgcttctctttgaaccataagatggcaaaaaattaaaccataagctgacaaatttcttagtgaacgtgttacaccacccaaaacgtggtaagccactgttgtaaaattacaagatatctcatgtacttcatagttacgaagtcggtttagctaagggcgagtcactcacgaccagttcaacagtgttacgggcactgtttaccagcgacctatgacgaagaccttcgatactttcaataatactactgtagagcatttcaaaggccatcgaaatatagaggtgtagttatattttaagcgtctcagagcaaaaaagccctctccatagcgctttcacattttcatccattctcgatctcgacaatgaaacttaactcccaaatcaacgagccagcggagaggagcaatttagctagctccgcgtgcattggcttg

SEQ ID NO: 137 - DNA, 479 bp, synthetic construct
  source 1..479: mol_type = other DNA; note = MG110-1-6 putative transposon end RE
aaccgagacctgtaagttgtaagtaaataacaacttaaatataggttatgccagctattactgcaaatttgacaacttatggttcaaatttgtcactttatgcttcaatttacatagctttaagttggtggcatctctgccagcctcaccccggcacccgagtctaccgctgtggctgcttccttccggacctgaccagattcacggtttatcgttgcggggggaccagcagagacaccatagaggaggtctcgttgaccttggtggggcattatacgcactctgtttcgatgtgcaacctacgctatggggtttattatcttttttctattagtgagaatactttaacgaaattcgctggttcgccgctcccgttgctgaagcttttagcgctctcggctatacttgactaactttatcggtttttgccgctgtctctgtatcacagagccggcgatgtattttatgggagcgcgcag

SEQ ID NO: 138 - DNA, 474 bp, synthetic construct
  source 1..474: mol_type = other DNA; note = MG110-2-6 putative transposon end LE
tgaggttgcctccagtacgtccacaccacgtggatgaccagacaggctttgaggttaactaattgatgttgttacaaccataagttgacataacggccataataaaatcaacaggttacaaaacgatttttacccataagttgacataaaaatgaagcataagctgacatagagtgacaagaccacacatggacacaaaatcggcgttgtaagtttacaagatcgacttgtagcatccactataggcaaacacacctacggcagccatcctctcattaagtgcaagtggcactatcgaacacaacgctcaccatttttgcgtagggcaaagtcgatccgccccgaattttctagaccatccgaacctttggctagcaggctacatgaatttctccatgtgccggatatcgagccattgtctgcataattataaaacgctccctattgcttgaaacgacttgcattgttttgagg

SEQ ID NO: 139 - DNA, 334 bp, synthetic construct
  source 1..334: mol_type = other DNA; note = MG110-2-6 putative transposon end RE
agcgcctccaacctgtagttgcttttctacaggtttaggctatgtcaacttatgatgcaagtttatgtcagcttttggttccactatgtcaacttatggttccaacgacaattgatttgccagaaatggtgggggctctgccagccacatcccggcactcgaaggactcgccttggctgctaccttccggtcctgaccaggtcgacgagattccgatgcgggaggaccaacagagcccccataacaacgccggcgagtctacctagcctgaccggcaattgcaatcgctttaccggtcagctgagttcaagtgtctgaaaactcgacaagtagc

SEQ ID NO: 140 - protein, 16 aa, synthetic construct
  source 1..16: note = Nucleoplasmin NLS
KRPAATKKAGQAKKKK

SEQ ID NO: 141 - protein, 19 aa, synthetic construct
  source 1..19: note = SV40 2x NLS
PKKKRKVDGSPKKKRKVDS

SEQ ID NO: 142 - protein, 19 aa, synthetic construct
  source 1..19: note = P2A
ATNFSLLKQAGDVEENPGP

SEQ ID NO: 143 - protein, 18 aa, synthetic construct
  source 1..18: note = T2A
EGRGSLLTCGDVEENPGP

SEQ ID NO: 144 - protein, 9 aa, synthetic construct
  source 1..9: note = HA
YPYDVPDYA

SEQ ID NO: 145 - protein, 10 aa, synthetic construct
  source 1..10: note = Myc
EQKLISEEDL

SEQ ID NO: 146 - protein, 8 aa, synthetic construct
  source 1..8: note = FLAG
DYKDDDDK

SEQ ID NO: 147 - protein, 640 aa, unidentified
  source 1..640: note = MG64-56 effector
MSVITIQCRLVADDKALRHLWELMAEKNTPLVNELLDRLGKHTDFEAWVQAGKVPKTTIKALCDSLKTQEPFIGQPGRFYTSATTLVAYIYKSWLALHKRRQRKIEGKERWLEMLKSDVELEQESNSSLELIRTIATEILSKFSASSTDGINQKSKGKKSKKLKKDKADEPMSIKPGVLFEAYQKTEDILRRSALVYLIKNNCQVNFAEEDPDKYAKMRRKKEIEIERLKEQLKSRVPKGRDLTGKKWLETLEKAVNSIPQDENEAKSWQAGLLRKSSTVPFPVAYETNEDMHWEISDKGRIFVSFNGLSKLKLEVYCDQRHLPWFQRFVEDQETKRKGKNQHSSGLFTLRSGRLSWLKQEGKGEPWSVNRLILFCSVDTRMWTVEGTQQVAIEKIADVEQNLTKAKEKGELNSNQQAFVTRQQSTLAKINTPFPRPSKPLYEGKSHILVGVSLGLENPATVAVFDAVNNKVLAYRSVKQLLGNNYNLLNRQQQQKQRLSHDRHKAQKDFARNDFGESELGQYVDRLLAKEIVAIAVTYFAGSIVLPKLGDMREIIQSEVQARAEKKIPGFKEGQQKYAKEYRKQVHNWSYGRLIENIQSQAAKVGILIETGQQPIRGSPQEQARDLALFAYQCRIASSI

SEQ ID NO: 148 - protein, 579 aa, unidentified
  source 1..579: note = MG64-56-B transposition protein
MGETLNSNEVDESLVLYDGSDEVDEISESEDTKQNNVIVTELSEEAKLRMEVLQSLIEPCDRKTYGIKLKQAAEKLGKTVRTVQRLVKKYQEQGLSGVTEVERSDKGGYRIDDDWQDFIVKTYKEGNKGGRKMTPAQVAIRVQVRAGQLGLEKYPCHMTVYRVLNPIIERKEQKQKVRNIGWRGSRVSHQTRDGQTLDVHHSNHVWQCDHTKLDVMLVDQYGETLARPWLTKITDSYSRCIMGIHLGFDAPSSLVVALAMRHAMLRKQYSSEYKLHCEWGTYGVPENLFTDGGKDFRSEHLKQIGFQLGFECHLRDRPPEGGIEERGFGTINTDFLSGFYGYLGSNVQERAEGAEEEACITLRELHLLIVRYIVDNYNQRIDARSGNQTRFQRWEAGLPALPNLVNERELDICLMKKTRRSIYKGGYVSFENIMYRGDYLSAYAGESVLLRYDPRDISTVFVYRQDSGKEVLLSQAHAIDLETEQISLEETKAASRKIRNAGKQLSNKSILAEVQDRDTFIKQKKKSHKERKKEEQAQVNFVKPPQTNEPVETVEEIPQPQKRRPRVFDYEQLRKDYDD

SEQ ID NO: 149 - protein, 278 aa, unidentified
  source 1..278: note = MG64-56-C transposition protein
MAEDYLRKWVQNLWGDDPIPEELLPIIERLITPSVVELEHIQKIHDWLDSLRLSKQCGRIVAPPRAGKSVTCDVYKLLNKPQKRTGKRDIVPVLYMQVPGECSAGELLTLILESLKYDAISGKLTDLRRRVLRLLKESKVEMLVIDEANFLKLNTFSEIARIYDLLKISIVLVGTDGLDNLIKKEPYIHDRFIECYRLPLVSEKKFPEFVQIWEDEVLCLPVPSNLTKRETLMPLYQKTSGKIGLVDRVLRRAAILSLRKGLKNIDKATLDEVLEWFE

SEQ ID NO: 150 - protein, 176 aa, unidentified
  source 1..176: note = MG64-56-Q transposition protein
MEIPAEQPRFFQVEPLEGESLSHFLGRFRRENYLTATQLGKLTGIGAVISRWEKFYLNPFPTPQELEALAAVVEVKVDRLIEMLPPRGVTMKPRPIRLCSACYQESPCHRVEWQFKDVMVCDCLRHCPLNNRHQLALLTKCTNCETPFPIPADWVQGECPHCFLPFTKMARRQKRY

SEQ ID NO: 151 - DNA, 485 bp, synthetic construct
  source 1..485: mol_type = other DNA; note = MG64-56 effector intergenic region encoding tracrRNA
caaaataccgaaccttgaaaacttaatatgaaagtaacagcgccgcagttcatgctcttctgagtctctgtactgtgataaatctgggttagtttaacggttgaaagaccgttttgctttctgaccctggtagctgctcgctcttgatgctgctgtcttttgacaggataggtgcgctcccagcaataaagagttaaagctgataaagcttgagccgttgtaaaacggtggggtttacctcagtggtggctactgaatcacccccttcgtcgggggaaccctcctaaatattttttttggcgtgtcaaagcgggggcaaaaatcctggagtcccgccaaaatctcaaaacctttgtcctatcttgacttgataaactagcatgtcagttaatttagttttttgatgtcaagtaggagatgcttttaggcagtcctgccaaagatgtgtatggaaagctctaatagcaagggttctagacggatcg

SEQ ID NO: 152 - DNA, 37 bp, synthetic construct
  source 1..37: mol_type = other DNA; note = MG64-56 effector target CRISPR repeat
gtttcaacaaccatcccagctaggggtgggttgaaag

SEQ ID NO: 153 - DNA, 595 bp, synthetic construct
  source 1..595: mol_type = other DNA; note = MG64-56 putative transposon end LE
tatgcgacttgagcgatttgtgtaagttttgtgttgcacaaaccatcctaagcgacattagtttgcaaaaaacgacattaatttacgaatcgcgacctttaatttgcgaatatacaacagattttgtcgattaactaattatttgtcgtcttaacaaattaatgtcgcccaaatcttcaagactataatccttatgtatcaaaggttatagccttttgaacttatattggctatcatcaaatatttaactaattaagtgtcgtcttttaattaattaacattttaaatgtcgttttttcaaaaaacacctttccaaatttttcttttgctcataacaaaataactgtcgtcttttggaagtgagtgaaaaatataaaattaaatgtcgctttttggaacaaagtagtatgatatttattaggcaatagtagctatgtaacaacaaaaacatagttagattgaagtcttcttttttgtctctagctacgaagtcattacccttgctgcgattaaatttagacgcaagctaatttcgctcttagacttgctgtaccgtattgcctaaccaactagtttcaagcgatgaagtttgttt

SEQ ID NO: 154 - DNA, 493 bp, synthetic construct
  source 1..493: mol_type = other DNA; note = MG64-56 putative transposon end RE
taagttctattaactccaagttttaataattgcatggcaataacaatcctttttagaaaggatttaagagggttgaaaggaatgtcaccttcccaagaatacttttcaaaagctattttgggttagggaagaataatcacagataactaatatgcacaagtaagtctaaaatagggataagtctgtcgattagtccaatagcaaggcatcttgttagacgacattaatttgttaacgttagttggaactaattcgacgacattaattcgttaacagcgacattaatttgttaatgacgacattaatctgttaacgacgacattaatctgttaacgacgacaaataatctgttaattgacagatttgaaagcgggtgatgggactcgaacccacgacgttcaccttgggaaggtgacattctaccactgaattacacccgcaaatggagtttaggctcaataaagctaacccccattataacacgatttgccca

SEQ ID NO: 155 - DNA, 568 bp, synthetic construct
  source 1..568: mol_type = other DNA; note = MG64-2 active transposon end RE
cctcgtgatacgcctatttttataggttaatgtcatgataataatggtttcttagacgtcaggtggcacttttcggggaaatgtgttctatctacaaaattgttttatacaactatgtttgtattttagttctcaggacaaagattgtgtcaattcttcacaaaagttgtaattctcgcctttttcaaaaaaggacaaagaatttgtcaactttcctaaaccggacaactaatttgtcaaaaagctttttcaatatataaagggggtataatagctgtaacccttgcgtagagtaggttagtgagctactaggtttggcggtcaataatttggcaagtggtcaaataatttggcaatcgacaattatttatcgttggggacgaatcagtgtcacggagtcaaattatgtgtcgttgtgacagattgatgtcgtctaagcaccagtgacaaattgatgttgctttaataatttaaaatcgtcacgaaataagcctaacgtgagttttcgttccactgagcgtcagaccaccgttgatgataccgctgccttactgggtgcattagccag

SEQ ID NO: 156 - RNA, 227 nt, synthetic construct
  source 1..227: mol_type = other RNA; note = MG64-4 effector putative tracrRNA
aatagcgccgcttgttcatgcgcgaaagcgtctctgaacagtgtaaatgtgggttagtttgactgtcgtgaagacggtcttgctttctgaccctggtagctgcccaccttgaagctgctgtctcttgtagacaggaatcaggtgcgcccccagtaatataggtgcgggtttaccgcagtggtggctacccaatcacctccgagcaaggaggaacccaccttaattat

SEQ ID NO: 157 - RNA, 228 nt, synthetic construct
  source 1..228: mol_type = other RNA; note = MG64-7 effector putative tracrRNA
aacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccaccttaattat

SEQ ID NO: 158 - RNA, 328 nt, synthetic construct
  source 1..328: mol_type = other RNA; note = MG64-9 effector putative tracrRNA
gctgaagttagtctctggatactgaagtcagtgtctagggactgaagtaaggggagctttacccgcaagggatactttcgaccccctgtagctgcccgctcctggtggggtgccctgacacccgcctcattacagcaatgtatgactgtctgggctaatgaaagaaggattagggacgcaggttcacgacctacttcaatcatagtctgtgcaacccagataagtgagtatgacccgcaagggtctaaacgcctttagtagaatggttcttccagtttgttaagggcagggcttggttctccaaggtggctacgaattcttctcgatt

SEQ ID NO: 159 - RNA, 187 nt, synthetic construct
  source 1..187: mol_type = other RNA; note = MG64-10 effector putative tracrRNA
ggtagcgctggagccacatagttcataagctcacgcttcttggacttcctgtgttctctaaaacgggttctgttttacccttaccaagggatactttcagatccgagtagctgcaagctcatggcggagtgtcccctgacgctttgccaccgtcatagcgatgtgatggccgtctggcgtatgaacg

SEQ ID NO: 160 - RNA, 326 nt, synthetic construct
  source 1..326: mol_type = other RNA; note = MG64-12 effector putative tracrRNA
aatagcgcccctaatcaaatcgaaggcgtttgaggacggggaaaatgggtaagtttccatcgaaaggtggttcttttcagccctatgtagtcccactcccttgtggagtgttagcatctgaggtgcctagcacagcaaagtctctcaggagacggagtcaaagctggggaacaagtattacgaacgaatctcgcgattctattagtatacgtaggtcgctcccatgcaacaagatgctcatctctcagcaatgagggtagggaacgtatcccaaacactacatgaatagctcgtcgggcttgcccggtgagtatccaccaagcctt

SEQ ID NO: 161 - RNA, 311 nt, synthetic construct
  source 1..311: mol_type = other RNA; note = MG64-13 effector putative tracrRNA
cgctaggtttgccttttcgcgcgctgctgaagcagtgaaaagttccgacagtttaaaagttctaacacttttactgctttccgtcgggagtagttgtccgcttctgcgttgttgagaccgaagtttgtccaggtgataggcagaacgttgatgcaacaggtagatagtcgcgctctatcaggaagctgttctagttattagtgttcgtgcactaataaagaggatacagggatacatgtggttgtgtccagcaatggcacaacagggccactactcgaagccccgagcaagggttgagcctacccaaattt

SEQ ID NO: 162 - RNA, 342 nt, synthetic construct
  source 1..342: mol_type = other RNA; note = MG64-14 effector putative tracrRNA
aacagcgccgcagtttaagctctatagccgctgaactgtgaaaaatgtgggtcagtttggtcgttgcaagacgatcgtgctttccgaccctagtaactgtccgctcactgactgccatcctggggcaaatcttcaaattttgtgtatttgtgtggggatggaaagctgcattagtcgattctcttcctccaatgtagcgtaggtgcgcacccagcagaagtgagttaagccttcacaatgtggaggtacagaagcatcatctctccattttttggtgtagatggtgtgactgaagtggtagttaccgaatcgcccctgatcaagggggagccctccataatt

SEQ ID NO: 163 - RNA, 297 nt, synthetic construct
  source 1..297: mol_type = other RNA; note = MG64-15 effector putative tracrRNA
aatgacgtttgtttctgtgcgcgctgctgaagcagtgaaaaatacggacagtctaatttgctttccgtccggagtagttgtccgcttctgcgttgttggaaccgaagtttgtccaggtgataggcggagtgtttcggcaacagatggagagtcgcacctctatcagggggcagttttagtggttagtgtttagcgcactatcactaggatacagggatacatgtggtagtgaccagagatggcactgcgggaccgctactcgaagccccgaacaagggttgagcttacccaaatctt

SEQ ID NO: 164 - RNA, 226 nt, synthetic construct
  source 1..226: mol_type = other RNA; note = MG64-16 effector putative tracrRNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaaaagggaacgggtttactggagtggtggtcgccgaatcacccccgagcaagggggactcgtcctttgc

SEQ ID NO: 165 - RNA, 227 nt, synthetic construct
  source 1..227: mol_type = other RNA; note = MG64-17 effector putative tracrRNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 166 - RNA, 227 nt, synthetic construct
  source 1..227: mol_type = other RNA; note = MG64-19 effector putative tracrRNA
aatagcgccgcagtttaagctcagcaagcctctggactgcgaaaagtatggggtagtttgaccgtcggtaaacggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagggggactcgtcctttgc

SEQ ID NO: 167 - RNA, 211 nt, synthetic construct
  source 1..211: mol_type = other RNA; note = MG64-20 effector putative tracrRNA
aatagcgccgcagtttaagctcaatgagcctcttgactgcgaacagtatggggtagtttgaccggcggtaaccggttgtgctttctgcccctggcgactgcccaccccgatgctgtcgatttctcaaccgggaatcgagaatcaggtgcgcccccagcaagagggaacgggtttactggagtggtggtcgccgaatcacccccgagcaagg

SEQ ID NO: 168 - RNA, 222 nt, synthetic construct
  source 1..222: mol_type = other RNA; note = MG64-21 effector putative tracrRNA
aatagcgccgcagttcatgcttctttgaagcctctgtgctgtgcaaaatgtgggttagtttggctgttgaagaaacagccttgctttctgaccctggtagctgtccaccctgaagctgctatcccctgtggataggataggtgcgcccccagcaataggggagcgggtataccgcagtggtggctactgaatcacctccaagcaaggaggaatccactttat

SEQ ID NO: 169 - RNA, 222 nt, synthetic construct; identical to SEQ ID NO: 168
  source 1..222: mol_type = other RNA; note = MG64-22 effector putative tracrRNA

SEQ ID NO: 170 - RNA, 248 nt, synthetic construct
  source 1..248: mol_type = other RNA; note = MG64-24 effector putative tracrRNA
aaaagcgccgtagaacatgctcacgcctctgttctgcgaaaaattagggtttgtttggctgtctgacagcagtcttactttctgtccctagaatctgaccactccgatgctgctgttgtaagtgaacttgattgagctcggacaccatgcaacaggataaggggcgcacccagcaagagaggacggacttaccgtagtgttggcttctgaagcaactccgaccaaggagtagtccatgcattcatcat

SEQ ID NO: 171 - RNA, 277 nt, synthetic construct
  source 1..277: mol_type = other RNA; note = MG64-25 effector putative tracrRNA
aatcgcgccgcacattcatgttccttgagaacctctgaattgcgaaagtgtgggctagtttgttcgcttgatgcgaatgtgctttctggccctggtagctgtccgccctgatgctgatttctacgggtaactgtaggaatgattaactcgttctatagacaggtttcgtgctttctattgtaacggggtcggtgcgctcccagcaataggggtgtgggtctactacagtgatggctactgaatcacctccgagcaaggaggaatccaccttaacttt

SEQ ID NO: 172 - RNA, 226 nt, synthetic construct
  source 1..226: mol_type = other RNA; note = MG64-27 effector putative tracrRNA
aatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 173 - RNA, 226 nt, synthetic construct; identical to SEQ ID NO: 172
  source 1..226: mol_type = other RNA; note = MG64-28 effector putative tracrRNA

SEQ ID NO: 174 - RNA, 255 nt, synthetic construct
  source 1..255: mol_type = other RNA; note = MG64-29 effector putative tracrRNA
tagcgcacgaacctgactagcgtcattacgatgcgagttctggaaatgggacagtttcattgctttccgtccctggcactgcctgcttactcacgaccacaaggagacaattcagcgctgtgagttcgcgattctctactgtttatgacaaactaacgtttgtctggcagtacagcaagattgtgtatcagaccatgtttggcgaagatacatggcaggccgaatcgccaatgaaaccagcggacttcccctaat

SEQ ID NO: 175 - RNA, 226 nt, synthetic construct
  source 1..226: mol_type = other RNA; note = MG64-32 effector putative tracrRNA
aatagcgccgcagtttaagctcaatgagcctcttgactgcgaacagtatggggtagtttgaccggcggtaaccggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcctttgc

SEQ ID NO: 176 - RNA, 203 nt, synthetic construct
  source 1..203: mol_type = other RNA; note = MG64-44 effector putative tracrRNA
tctagcgccgcagctcatgtcagcaatggccaatgtgttgtgctaaatgcgagctagtttgactgcctgctaagcagtcttgctttctggctcaggtgactatccacccaaaggtcgttggtgcgctggcgatttgagggcacgggttccggagtgatagttaccattacacctccggccaaggaggaatccaccccaccccc

SEQ ID NO: 177 - RNA, 203 nt, synthetic construct; identical to SEQ ID NO: 176
  source 1..203: mol_type = other RNA; note = MG64-46 effector putative tracrRNA

SEQ ID NO: 178 - RNA, 310 nt, synthetic construct
  source 1..310: mol_type = other RNA; note = MG64-49 effector putative tracrRNA
gattgcgcctcgatcgatgctctatgagccgctcgatcgtagaaaaatgggtgagtttgattatctacttcgttagataatgctgctttccgaccctggcattctgtccgcccttgaagctgcttctcatggactagcgtaagctcgttggtaagaaggaaaagtcataatttaaagtcacgtctttctagtatgacataggtgcgctcccacgcaatatagggttcagcttttattttataaaagtagagactttcctctagtgacagtgccgaaatgaccccgtgcgaggggtaactacctaagtttt

SEQ ID NO: 179 - RNA, 227 nt, synthetic construct
  source 1..227: mol_type = other RNA; note = MG64-51 effector putative tracrRNA
aacagcgccgcagttcatgtttgttataaacctctgtactgcgataaatgcgggttagtttgactgttgtgagacagtcttgctttctgaccctagtagctgcccaccttgatgctgctgttcccagtgaacaggaataaggtgcgcccccagtaatagaggtgcgggtttaccgcagtggtggctactgaatcacctccgactaaggaggaatccaccttaattat

SEQ ID NO: 180 - RNA, 380 nt, synthetic construct
  source 1..380: mol_type = other RNA; note = MG64-52 effector putative tracrRNA
accggggatcgtcctggtgaagggagagttgctttggatcggtcacaagcttttccttaactaattctcactgactgactaggatgatcgagggggttatgttttaccactgcaaggtggatactttcaaacccctgtggtagctgctcgctcctggtgaggtgccctgacacttcaccccactacagcaatgtgtgtggctgtctggttatgagagaagttagggcataggttcgttacctgcattcaatcataaattacgcgaccagataagtgagtatgatccgcaaggatctatatgtctttagcaaagaagtgcttctgctttgttactggcgtagggcatggttctctaaagtggctaccgaaccttcccaatt

SEQ ID NO: 181 - RNA, 257 nt, synthetic construct
  source 1..257: mol_type = other RNA; note = MG64_56 effector putative tracrRNA
aacagcgccgcagttcatgctcttctgagtctctgtactgtgataaatctgggttagtttaacggttgaaagaccgttttgctttctgaccctggtagctgctcgctcttgatgctgctgtcttttgacaggataggtgcgctcccagcaataaagagttaaagctgataaagcttgagccgttgtaaaacggtggggtttacctcagtggtggctactgaatcacccccttcgtcgggggaaccctcctaaatatt

SEQ ID NO: 182 - RNA, 246 nt, synthetic construct
  source 1..246: mol_type = other RNA; note = MG108-1 effector putative tracrRNA
tttagcgcacttgttcgagttcgtttgaactgaacaagggtaagtatgggccagtttaattgctttccgtcccaggatagctgccagcttctaccgtaggttcgtcctgcaagtgatgctaagtcgcgcctagcatcaaggagctatgtcttgattgtcttgggtgtccgccctggatgagttgaggtgtagatgcttctatcatggcagctactaaacgccccaagcaaggggaacccatcttta

SEQ ID NO: 183 - RNA, 223 nt, synthetic construct
  source 1..223: mol_type = other RNA; note = MG108-2 effector putative tracrRNA
aatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccttaactat

SEQ ID NO: 184 - DNA, 41 bp, synthetic construct
  source 1..41: mol_type = other DNA; note = MG64-9 effector target CRISPR repeat
gtctttcatcctatctcgcgccagatcgcttcctgcaaccc

SEQ ID NO: 185 - DNA, 37 bp, synthetic construct
  source 1..37: mol_type = other DNA; note = MG64-12 effector target CRISPR repeat
gttgcaagcgcctccttggctgttggtgggtggaaag

SEQ ID NO: 186 - DNA, 37 bp, synthetic construct
  source 1..37: mol_type = other DNA; note = MG64-14 effector target CRISPR repeat
gttgcaatcgccttcccagagatgggtgggctgaaag

SEQ ID NO: 187 - DNA, 38 bp, synthetic construct
  source 1..38: mol_type = other DNA; note = MG64-15 effector target CRISPR repeat
gttacaattaccctcccagcgttgggtgggttgaaagg

SEQ ID NO: 188 - DNA, 39 bp, synthetic construct
  source 1..39: mol_type = other DNA; note = MG64-16 effector target CRISPR repeat
aagttgcatccgctttccagcaaccagggcgggtgaaag

SEQ ID NO: 189 - DNA, 37 bp, synthetic construct
  source 1..37: mol_type = other DNA; note = MG64-17 effector target CRISPR repeat
gttgcatccgctttccagcaaccagggcgggtgaaag

SEQ ID NO: 190 - DNA, 33 bp, synthetic construct
  source 1..33: mol_type = other DNA; note = MG64-19 effector target CRISPR repeat
gtacccaaagccttttttccttaagcctatccg

SEQ ID NO: 191 - DNA, 27 bp, synthetic construct
  source 1..27: mol_type = other DNA; note = MG64-25 effector target CRISPR repeat
gtttcaaccgccatcccagctaggggt

SEQ ID NO: 192 - DNA, 38 bp, synthetic construct
  source 1..38: mol_type = other DNA; note = MG64-27 effector target CRISPR repeat
agttgcatccgctttccagcaaccagggcgggtgaaag

SEQ ID NO: 193 - DNA, 38 bp, synthetic construct
  source 1..38: mol_type = other DNA; note = MG64-28 effector target CRISPR repeat
gttgcatctgcttttcagcaactagggcgggggaaagc

SEQ ID NO: 194 - DNA, 37 bp, synthetic construct
  source 1..37: mol_type = other DNA; note = MG64-29 effector target CRISPR repeat
ggcgcgatcgcctttatgggtacgggcaagttgaaag

SEQ ID NO: 195 - DNA, 41 bp, synthetic construct
  source 1..41: mol_type = other DNA; note = MG64-32 effector target CRISPR repeat
aagttgcatccgctttccagcaaccagggcgggtgaaagtt

SEQ ID NO: 196 - DNA, 37 bp, synthetic construct
  source 1..37: mol_type = other DNA; note = MG64-44 effector target CRISPR repeat
gttgcctcccgcttcgaggcacgggaacgattgaaag

SEQ ID NO: 197 - DNA, 37 bp, synthetic construct; identical to SEQ ID NO: 196
  source 1..37: mol_type = other DNA; note = MG64-46 effector target CRISPR repeat

SEQ ID NO: 198 - DNA, 43 bp, synthetic construct
  source 1..43: mol_type = other DNA; note = MG64-49 effector target CRISPR repeat
gttgcaacactccctgactgcctgacacaaatgcctcgaaagc

SEQ ID NO: 199 - DNA, 34 bp, synthetic construct
  source 1..34: mol_type = other DNA; note = MG64-52 effector target CRISPR repeat
gtcgcaatgactattttggcttggggcggaatga

SEQ ID NO: 200 - DNA, 37 bp, synthetic construct
  source 1..37: mol_type = other DNA; note = MG64-51 effector target CRISPR repeat
gtttcaacacccctcccgaagtggggcgggttgaaag

SEQ ID NO: 201 - RNA, 261 nt, synthetic construct; identical to SEQ ID NO: 111
  source 1..261: mol_type = other RNA; note = MG64-6 active effector sgRNA

SEQ ID NO: 202 - RNA, 263 nt, synthetic construct; identical to SEQ ID NO: 112
  source 1..263: mol_type = other RNA; note = MG64-2 effector sgRNA

SEQ ID NO: 203 - RNA, 265 nt, synthetic construct; identical to SEQ ID NO: 113
  source 1..265: mol_type = other RNA; note = MG64-4 effector sgRNA

SEQ ID NO: 204 - RNA, 276 nt, synthetic construct; identical to SEQ ID NO: 114
  source 1..276: mol_type = other RNA; note = MG64-7 effector sgRNA

SEQ ID NO: 205 - RNA, 246 nt, synthetic construct; identical to SEQ ID NO: 115
  source 1..246: mol_type = other RNA; note = MG108-1 effector sgRNA 1

SEQ ID NO: 206 - RNA, 274 nt, synthetic construct; identical to SEQ ID NO: 116
  source 1..274: mol_type = other RNA; note = MG108-1 effector sgRNA 2

SEQ ID NO: 207 - RNA, 60 nt, synthetic construct; identical to SEQ ID NO: 121
  source 1..60: mol_type = other RNA; note = MG110-1-6 effector crRNA

SEQ ID NO: 208 - RNA, 59 nt, synthetic construct; identical to SEQ ID NO: 122
  source 1..59: mol_type = other RNA; note = MG110-2-6 effector crRNA

SEQ ID NO: 209 - RNA, 227 nt, synthetic construct; identical to SEQ ID NO: 156
  source 1..227: mol_type = other RNA; note = MG64-4 effector putative tracrRNA

SEQ ID NO: 210 - RNA, 228 nt, synthetic construct; identical to SEQ ID NO: 157
  source 1..228: mol_type = other RNA; note = MG64-7 effector putative tracrRNA

SEQ ID NO: 211 - RNA, 328 nt, synthetic construct; identical to SEQ ID NO: 158
  source 1..328: mol_type = other RNA; note = MG64-9 effector putative tracrRNA

SEQ ID NO: 212 - RNA, 187 nt, synthetic construct; identical to SEQ ID NO: 159
  source 1..187: mol_type = other RNA; note = MG64-10 effector putative tracrRNA

SEQ ID NO: 213 - RNA, 326 nt, synthetic construct; identical to SEQ ID NO: 160
  source 1..326: mol_type = other RNA; note = MG64-12 effector putative tracrRNA

SEQ ID NO: 214 - RNA, 311 nt, synthetic construct; identical to SEQ ID NO: 161
  source 1..311: mol_type = other RNA; note = MG64-13 effector putative tracrRNA

SEQ ID NO: 215 - RNA, 342 nt, synthetic construct; identical to SEQ ID NO: 162
  source 1..342: mol_type = other RNA; note = MG64-14 effector putative tracrRNA

SEQ ID NO: 216 - RNA, 297 nt, synthetic construct; identical to SEQ ID NO: 163
  source 1..297: mol_type = other RNA; note = MG64-15 effector putative tracrRNA

SEQ ID NO: 217 - RNA, 226 nt, synthetic construct; identical to SEQ ID NO: 164
  source 1..226: mol_type = other RNA; note = MG64-16 effector putative tracrRNA

SEQ ID NO: 218 - RNA, 227 nt, synthetic construct; identical to SEQ ID NO: 165
  source 1..227: mol_type = other RNA; note = MG64-17 effector putative tracrRNA

SEQ ID NO: 219 - RNA, 227 nt, synthetic construct; identical to SEQ ID NO: 166
  source 1..227: mol_type = other RNA; note = MG64-19 effector putative tracrRNA

SEQ ID NO: 220 - RNA, 211 nt, synthetic construct; identical to SEQ ID NO: 167
  source 1..211: mol_type = other RNA; note = MG64-20 effector putative tracrRNA

SEQ ID NO: 221 - RNA, 222 nt, synthetic construct; identical to SEQ ID NO: 168
  source 1..222: mol_type = other RNA; note = MG64-21 effector putative tracrRNA

SEQ ID NO: 222 - RNA, 222 nt, synthetic construct; identical to SEQ ID NO: 168
  source 1..222: mol_type = other RNA; note = MG64-22 effector putative tracrRNA

SEQ ID NO: 223 - RNA, 248 nt, synthetic construct; identical to SEQ ID NO: 170
  source 1..248: mol_type = other RNA; note = MG64-24 effector putative tracrRNA

SEQ ID NO: 224 - RNA, 277 nt, synthetic construct; identical to SEQ ID NO: 171
  source 1..277: mol_type = other RNA; note = MG64-25 effector putative tracrRNA

SEQ ID NO: 225 - RNA, 226 nt, synthetic construct; identical to SEQ ID NO: 172
  source 1..226: mol_type = other RNA; note = MG64-27 effector putative tracrRNA

SEQ ID NO: 226 - RNA, 226 nt, synthetic construct; identical to SEQ ID NO: 172
  source 1..226: mol_type = other RNA; note = MG64-28 effector putative tracrRNA

SEQ ID NO: 227 - RNA, 255 nt, synthetic construct; identical to SEQ ID NO: 174
  source 1..255: mol_type = other RNA; note = MG64-29 effector putative tracrRNA

SEQ ID NO: 228 - RNA, 226 nt, synthetic construct; identical to SEQ ID NO: 175
  source 1..226: mol_type = other RNA; note = MG64-32 effector putative tracrRNA

SEQ ID NO: 229 - RNA, 203 nt, synthetic construct; identical to SEQ ID NO: 176
  source 1..203: mol_type = other RNA; note = MG64-44 effector putative tracrRNA

SEQ ID NO: 230 - RNA, 203 nt, synthetic construct; identical to SEQ ID NO: 176
  source 1..203: mol_type = other RNA; note = MG64-46 effector putative tracrRNA

SEQ ID NO: 231 - RNA, 310 nt, synthetic construct; identical to SEQ ID NO: 178
  source 1..310: mol_type = other RNA; note = MG64-49 effector putative tracrRNA

SEQ ID NO: 232 - RNA, 227 nt, synthetic construct; identical to SEQ ID NO: 179
  source 1..227: mol_type = other RNA; note = MG64-51 effector putative tracrRNA

SEQ ID NO: 233 - RNA, 380 nt, synthetic construct; identical to SEQ ID NO: 180
  source 1..380: mol_type = other RNA; note = MG64-52 effector putative tracrRNA

SEQ ID NO: 234 - RNA, 257 nt, synthetic construct; identical to SEQ ID NO: 181
  source 1..257: mol_type = other RNA; note = MG64-56 effector putative tracrRNA

SEQ ID NO: 235 - RNA, 246 nt, synthetic construct; identical to SEQ ID NO: 182
  source 1..246: mol_type = other RNA; note = MG108-1 effector putative tracrRNA

SEQ ID NO: 236 - RNA, 223 nt, synthetic construct; identical to SEQ ID NO: 183
  source 1..223: mol_type = other RNA; note = MG108-2 effector putative tracrRNA

SEQ ID NO: 237 - RNA, 41 nt, synthetic construct
  source 1..41: mol_type = other RNA; note = MG64-9 effector crRNA
gtctttcatcctatctcgcgccagatcgcttcctgcaaccc

SEQ ID NO: 238 - RNA, 37 nt, synthetic construct
  source 1..37: mol_type = other RNA; note = MG64-12 effector crRNA
gttgcaagcgcctccttggctgttggtgggtggaaag

SEQ ID NO: 239 - RNA, 37 nt, synthetic construct
  source 1..37: mol_type = other RNA; note = MG64-14 effector crRNA
gttgcaatcgccttcccagagatgggtgggctgaaag

SEQ ID NO: 240 - RNA, 38 nt, synthetic construct
  source 1..38: mol_type = other RNA; note = MG64-15 effector crRNA
gttacaattaccctcccagcgttgggtgggttgaaagg

SEQ ID NO: 241 - RNA, 39 nt, synthetic construct
  source 1..39: mol_type = other RNA; note = MG64-16 effector crRNA
aagttgcatccgctttccagcaaccagggcgggtgaaag

SEQ ID NO: 242 - RNA, 37 nt, synthetic construct
  source 1..37: mol_type = other RNA; note = MG64-17 effector crRNA
gttgcatccgctttccagcaaccagggcgggtgaaag

SEQ ID NO: 243 - RNA, 33 nt, synthetic construct
  source 1..33: mol_type = other RNA; note = MG64-19 effector crRNA
gtacccaaagccttttttccttaagcctatccg

SEQ ID NO: 244 - RNA, 27 nt, synthetic construct
  source 1..27: mol_type = other RNA; note = MG64-25 effector crRNA
gtttcaaccgccatcccagctaggggt

SEQ ID NO: 245 - RNA, 38 nt, synthetic construct
  source 1..38: mol_type = other RNA; note = MG64-27 effector crRNA
agttgcatccgctttccagcaaccagggcgggtgaaag

SEQ ID NO: 246 - RNA, 38 nt, synthetic construct
  source 1..38: mol_type = other RNA; note = MG64-28 effector crRNA
gttgcatctgcttttcagcaactagggcgggggaaagc

SEQ ID NO: 247 - RNA, 37 nt, synthetic construct
  source 1..37: mol_type = other RNA; note = MG64-29 effector crRNA
ggcgcgatcgcctttatgggtacgggcaagttgaaag

SEQ ID NO: 248 - RNA, 41 nt, synthetic construct
  source 1..41: mol_type = other RNA; note = MG64-32 effector crRNA
aagttgcatccgctttccagcaaccagggcgggtgaaagtt

SEQ ID NO: 249 - RNA, 37 nt, synthetic construct
  source 1..37: mol_type = other RNA; note = MG64-44 effector crRNA
gttgcctcccgcttcgaggcacgggaacgattgaaag

SEQ ID NO: 250 - RNA, 37 nt, synthetic construct; identical to SEQ ID NO: 249
  source 1..37: mol_type = other RNA; note = MG64-46 effector crRNA

SEQ ID NO: 251 - RNA, 43 nt, synthetic construct
  source 1..43: mol_type = other RNA; note = MG64-49 effector crRNA
gttgcaacactccctgactgcctgacacaaatgcctcgaaagc

SEQ ID NO: 252 - RNA, 34 nt, synthetic construct
  source 1..34: mol_type = other RNA; note = MG64-52 effector crRNA
gtcgcaatgactattttggcttggggcggaatga

SEQ ID NO: 253 - RNA, 37 nt, synthetic construct
  source 1..37: mol_type = other RNA; note = MG64-51 effector crRNA
gtttcaacacccctcccgaagtggggcgggttgaaag

SEQ ID NO: 254 - RNA, 37 nt, synthetic construct
  source 1..37: mol_type = other RNA; note = MG64-56 effector crRNA
gtttcaacaaccatcccagctaggggtgggttgaaag

SEQ ID NO: 255 - RNA, 37 nt, synthetic construct
  source 1..37: mol_type = other RNA; note = MG64-2 effector crRNA
gtttcaacgaccatcccaactaggggtgggttgaaag

SEQ ID NO: 256 - RNA, 37 nt, synthetic construct
  source 1..37: mol_type = other RNA; note = MG64-4 effector crRNA
gtttcaactttccttccagctagaggcgggttgaaag

SEQ ID NO: 257 - RNA, 37 nt, synthetic construct
  source 1..37: mol_type = other RNA; note = MG64-6 effector crRNA
gtttcaaccaccatctcaactagggatgggttgaaag

SEQ ID NO: 258 - RNA, 38 nt, synthetic construct
  source 1..38: mol_type = other RNA; note = MG64-7 effector crRNA
gtttcaacgccccttcaagctttgggcgggttgaaagc

SEQ ID NO: 259 - RNA, 36 nt, synthetic construct
  source 1..36: mol_type = other RNA; note = MG64-13 effector crRNA
tggcaattgcccttccagtgttgggtgggttgaaag

SEQ ID NO: 260 - RNA, 37 nt, synthetic construct
  source 1..37: mol_type = other RNA; note = MG108-2 effector crRNA
gtttcaacgaccatcccgacaaggggtgggttgaaag

SEQ ID NO: 261 - RNA, 37 nt, synthetic construct
  source 1..37: mol_type = other RNA; note = MG108-1 effector crRNA
gttgcgatcgccgctccggtggcgatggggttgaaag

SEQ ID NO: 262 - RNA, 222 nt, synthetic construct
  source 1..222: mol_type = other RNA
aattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccact

SEQ ID NO: 263 - RNA, 237 nt, synthetic construct
  source 1..237: mol_type = other RNA
ataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccttaattttttatttttt

SEQ ID NO: 264 - protein, 633 aa, unidentified
  source 1..633: note = MG64-57 effector
MSIITIQCRLVASESARRYLWQIMSEKNTPLVNELLKQVGKHSDFEKWLQAGKLPPGVVRSVCNLLASQECFAEQPKRFYKSAIELTEYIYKSWLALQQRRQKQLDGKTHWLSMLKTDAELVEISGCSLENIRAKALEILVRITTHPSNQPQQKKKSKKGSNSKVSSKLSTTLFQAYDQTEDEPERCAITYLLKNNCQVTEEEEDAKKFALRRRKKEKEIERLKEQLASRMPHGRDLTGEQWLQTLLIAKTTVPQSEEEARSWQSSLLKKPSSIPFPVQFTSKEDLIWSKNQNGRICIKFSGLGEHIFEVYCDRRQLHWFQRFLEDQQIKRSGKDQYSSGLFTLCSGRLAWQENEGRGEPWNVHCLTLYCSLDTRLWTIEGTETVSQDKAAEVAKELTKMQDKGDLNPNQQNYIKRLSSTLTKINNPYPRPSKPLYQGQTSILVGVSLGLEKPATAAVVDASTNTVLAYRSLKQLLDENYKLLNRQRQQQQRNAHERHKAQKRSTPNQLSESELGKHLDNLLAKAIITLAQTYQAGSIIVPNIKNVREGIHSEIEAKAENKCPNFKEGQQKYAKQYRQTIHRWSYNRLIDCIHSQAAKAKIPVEQAPQPIRGSPQEKARSIAIAAYHSRQNKS

SEQ ID NO: 265 - protein, 713 aa, unidentified
  source 1..713: note = MG64-58 effector
MSQITIQCRLVASASTRQKLWKLMAELNTPLINELLILAYQHPDFETWQHKGAIPAGIIKQLCEPLKTDARFVGQPGRFFASAIATVSYIYKSWVKVQKRLQLQIDGKTRWLEMLNSDTELVEMAGVALDTLRATATEFLNQLNPQPTTEESPKKKGKKTKKTQQLQGERSLSKILFDTYSDTEDVQTRCAISYLLKNGCKIPNKEEDSKKFAQRRRKVEIQIQRLTDQLASRVPKGRDLTGAKWLECLLEAVRKVPKNEAEAKSWQDSLLRQSSSLPFPVAFESSEDMTWFRTLKLNNIPIKLWTLLLYIDYLTVISFVRDSLQNEALWFKNLKINNIDVLKKVWMILLNINSFAGILFLDGVLKKYQKRICVHFNGLSDLMFEIYCDSRHLHWFKRFLEDQQIKRSSKNQHSSSLFTIRSGRIAWKSTQGKGKPWNVNRLMLYCCVDTRLWTAEGTNLVVEEKALEIAKTITRTKEKETKEQVQLNDKQLAYIKRKNATLTRINSPFPRPSKPLYSGQSHILVGVSLGLEKPATLAVLNAITGKIIAYRSVKQLLGKNYKLINRQRHEKLALSHQRKVAQTLAAPNEFGDSELGEHIDRLLAKEIIAVAQKFNAGSIVVPNLDNMREQVNSEIQAKAEEKCPESIEAQKKYASSYRRSVNQWSYRRLIDCITNQAAKAGIVIEENKQPIRASPQDKAKELALSAYNARKKS

SEQ ID NO: 266 - protein, 650 aa, unidentified
  source 1..650: note = MG64-59 effector
MLLLNIFVHWLFDMSQITIQCRLIASVYIRQNLWKLMAELNTPLINELLMQMRQHPDFETWRQKGKISTVVVKQLCEPLKTDHRFTGQSARFYTSAVTTVSYIYKSWLALMKRTQYQLEGKIRWLEMLNSDTELIEASGVSLDSLHTKAAEILLQLAPIDTAETQPVQRKKVKKGKKSQNSDSERNLSKNLFDAYGNTEDNLTRCAISYLLKNGCKISDKEENPDKFAQRRRKLEIQIQRLTEQLEARVPKGRDLTDTKWLETLYTAAQTVPENEAEAKLWQNILLRKSSKVPFPVAYETNEDMTWFKNQFGRICVKFNGLSEHSFQVYCDSRQLHWFKRFLEDQQIKKDSKNQHSSALFTLRSGCISWQEEEGKEEPWNIHHLTLSCCVDTRLWTEEGTKLVKEEKAEEIAKTITQTKAKGDLNVQQQAHIKRKNSSLARINNPFPRPSKSLYKGQPHILLGVSLGLEKPATVAVVDVVLGKVLTYRNIKQLLGDNYKLLNRQRQQKQALSHARQIAQTQASPNQYGDSELGEYIDRLLAKEIIAIAQKYSATSIVLPKLDGMREQVNSEIQAKAEQKCPESIEAQKKYAKQYRRSVNQWSYGRLIENIKSQSAQAGIAIEEAKQLIQGSPQEKAKELAFVAYNSRKKS

SEQ ID NO: 267 - protein, 700 aa, unidentified
  source 1..700: note = MG64-61 effector
MKKKDSETLITIQCRLVAEENTLRQLWELMADKYTPLINELLVQVGQHPDFDKWLKQGEVSKEAIETIKKSLITQEPFAGQPGRFYKSAVILVEEIYKSWFALQQKRQRKIEGKERWLKMFKSDIELQKKSQCSLDILRNKAKEILSSFSTKSNQPIKQISKTQKRKNKEEVEYSTLFTSLFDLYDKTENCLSKYAIAYLLKNDCQVSEIDEEQVQYAKRRRRKEIQVERLKEQLNSQKPKGRDLTGEKWLSTLKETTNQVPLDEFEAKSWQDSLLTQTSNIPYPVDYETNTDLDWFIHSADDEIKRKIIIVWQIYFLNELIKSGAYSLIKHLYFKMGCLPQKDVNCLNLQNKPGRIFIKFNGLKKNVNNPEFYICCDSRQLHYYQRFCQDWQVWHDKEINYSSALFVLRSARLLWQERKGKGAPWNVHRLILQCSIETRLWTKEEMELVRTEKIAQAEKTIRNMEQKTDLTKKQLSRLKGERTLRQALEQPSPHCPSKSLYQGKSNIIVGVSLGLEKPVTLAVVDVVKNKVLAYRSVKQLLAENYNLLNRQRQQQQRLSHERHKAQKQNAPNSFGESELGQYVDRLLADAIIAIAQKYQAGSIVLPKLRDMREQISSEIQSRAENRCPGYKEGQQKYAKEYRISVHRWSYGRLTESIKSQAVQAGISIEIGTQPIRGSPQEKARDLALFSYQERQALLI

SEQ ID NO: 268 - protein, 641 aa, unidentified
  source 1..641: note = MG64-60 effector
MGFITIQCRLVASKAVRLLLWYLMASKNTPLVNELLKQASQHDDFETWQRKGTVPEKSVQNLCKLLKTHPDFAGQPGRFYTSASLMVIYIYQSWLALQQKRRQRLDGKQRWLSVVKSDIELVRISNCSLQTIQDKAREILAQHNAQIGVDPDQTKQKSKKKQPLEANGNLRRSLFQAYETTEDVLSQCAIVYLLKNECQVREQEENPEAFAQRIHRKQKEIERLEAQLKSRLPKGRDLTGEEFLQTLSTATGRVPADDIEQMLWQAKLLAKPATLPYPIIFGSQTDLRWSINKKGRICVSFNGLDKAIPELKENPLQIYCDQRQLPLFQRFLEDWQAYQTNKNTYPSGLSLLKTGMLSWQEGTEKGEPWQANHLTLHCTINTRCLTVEGTEQLRQDGIIRLRKQLAEGKKSEELTENQQDFIKRQKSTLTRLENFSQLPKKSLYQGQPDILVGVSIGLAHPITISVVNARTGDVLTYRSTSQLLGDNYRLLNRQRQQQQRNTLKRHKNQVKGYTHQPSESELGQYVDRLLAESVIEVAQKFQASCIVLPHTNNLREHLAAEIKAKAERKSDLVEVQDKYAKQYRINIHRWSYDRLLSTIGAKADKMGLAIETTAQPHQGSPQEKARDIAVAAYNFRQVALN

SEQ ID NO: 269 - protein, 801 aa, unidentified
  source 1..801: note = MG64-62 effector
MAVSRPKPSEKKKKLDELYKVVCCWLCVDEATRKIIWEAMEKYTLLVNLLTERVAQLPEFEQWRQQGWVPEKAVMGMCQALKQEEQFKGLPSRFYMSAQSMVKDTLDGWLKLQQRLSRKINGKKRWLKLVEEDIELATVTDFDPEVIQNRAKEILLELSQPENSEPQLSESLEEAPDESQQEDGSRDGQQSPFSLLFDLYEIAEDCLSRRAIVHLLKNGGEVNEETEDLEKLTQKLATKREAIQRLEKQLVSRLPKGRDPTGEQALQFLQEAIQFPEHSNRYPSLFLFDIFYHYASLPMPYVQYAAYLLQAILAESKIVESEYLEWQGAMTERFHNAAKIPNSLPYPLLFGSTDDLYWSLESRGGSQQNSQPGKPCKGAKSRKRPSKRQRKKLKTRSEERICVRFKGLGNYVFKIYCDRRQLPIFRLFATDWLTYKELDKDSKYSLGLFALRSAKLIWKEDEQLLYKKGKSKQANSGSTGISSELEALPPWKAHRLCLHCSIDPALLTAEGTERVRSRKLSETKKTLDKAREKEQKLLVQISEVELAEEEKQKLENELAKRRSRILSYETSLIQLNNSPPRPSKVPYQGEANITLGISFCRQELVGVAVVDLQSQQVLEYYSIWKLLANQRVKKPRRDRSLLQLNLEKYRLVNRLRKLSKRNLAYRREEQKQGQYVESKAESNLGQYLERLIAARIAQVALEWRASSVTIPDLGDMREYIESTVQARAKQLFPNHREQQKNYAKQFRIEGHRWSYAMLAEFICSRTLSEGITVETGRQSTTGNLVNKALNVALSAHSLNLT

SEQ ID NO: 270 - protein, 635 aa, unidentified
  source 1..635: note = MG64-63 effector
MSQSTIQCQLTANEATRSYLWALMAEKNTPLINELIGQVTKHSDFETWKLKGKIPSSVVSQLCQPLKTDPRFIGQPARFYTSATHVVDYTFKSWLALQKRLSDTLQRKIRWSEMLKSDPELVELSGCNLATIRAKAAEILAVATNKSQQGGTEPSQKTRKSKQPKDSAPGRTVSSLLFEAHRNTQDILSQCAICYLLKNSCKVSDREEDPDKFAKRRRKVEIQIQRLQDQIEGRLPKGRDLTGQSWLNTLAIATSTVPVDNAEAKRWQDKLLTQSTSLPFPVSFETNEDLVWSNNQKGRISVRLNGLGEHTFQIACDQRQLPWFKRFLEDQQTKREGKNQHSSALFALRSGRLAWQEGKEKGEPWKANRLILYCTVDTRLWTAEGTEQVRNEKAIEIAQTLTRMKEKEGLSQTQQDYIKRLDSSLTRINSPFDRPSQSLYQPLPHILVGICLGLEQPATVVVLDINTNQVLTHRTVRQLLGRNYALLLKRRREQQRTAHQRHKAQKRDAPRSLGESELGQYVDRLLAKATVELARTHRASSIVVPKLGDMREIVQSEIQAKAEQKIPGSIEAQQKYAKQYRINVHRWSYSRLIESIRAQAAKDGIALEEGRQPLQGSLEERAKGVAIAAYQSRKK

SEQ ID NO: 271 - protein, 504 aa, unidentified
  source 1..504: note = MG64-64 effector
CWLNVLKSDDELVELAGKPITDIYSQANEILSRFGDSVNRDSIFRTLFQRFKDSGDSPSRWAIAFLLKNGCKLPLEIEDVNKFAKRRRKVEIQIERLTAQLEARLPKGRDLTGQKWLDTLVVAATTMPESEIEAHRWQSELLKKPRTIPFPLIFETNEDLSWFKNAKERLCMHLPGLREHSFEIYCDQRQLHWFKRFLEDQKVKRQGKDQHSSGLFTLRSTRLSWLEGKGKGEPWNIHRLALYCTVDTRLWTMEGTEQVRQEKVTEILKVIDHAKTKSSLTKTQQSFVQRKESTLAKINNPFPRPSQQLYIGRSNILVGVSIGLQTPATIAVVDGITGKVLVYRSVKQLLGDRYNLLNRHRQQQRQNTHRRHNAQKRFNSNQFETTDLGQYLDELLAQAIVKVAQKYQAGSIVVPKLENLREVIQSEIQTKAEQKLPGFVELQKQYAKQYRINIHAWSYNRLIQSIQSQALQANLCIEECVLDKQGNPQEKAKALAIAAYKARC

SEQ ID NO: 272 - protein, 639 aa, unidentified
  source 1..639: note = MG64-65 effector
MSVITIQCRLVAEEETLRHLWELMAEKNTPLANEILERLAKHDDFKTWVEDAIVPKTVIKELCDFLKNQEPFAGQPGRFYTSATTLVKYIYKSWLKLNRQLQRKIQGKERWLNMLKSDAELEKESNSTIETIRQKASEILISLNAQRTKNIELKSIKPKNQKQVQTVQSPKITSSVLFESYLQAEDTLTQCAIVYLLKNNFKINSNEEDIDKYLKQRRKKEIEIERLKDQLKSRVPKGRDLTGERWLSTLEQAVSTTFKDENEAKSWQAGLLRKSSNLPFPVLYETNEDMKWEMNDKGRLFVSFNGLSKLKFEVFCDKRHLYLFHRFLEDQETKRQGKNQHSSALFTLRSGRIAWSEQAGKGQPWNLSRLHLFCSLDTLMLTSQGTQQVIEKKITGVQDKLAKAQEKEKEEGGLNSQQQADVIRKKSTLAKIKTPFSRPSKPLYQGKSHIIVGVSLGLEKLATIAVFDAVSNKVLAYRSTKQLLGSKYNLLNRQRQQKKRLSQERHKSQKQFAPNNFGESALGQYVDRLLAKEIVAVAKTYGAGTIVVPKLSDMREIIQSEVQAKAENKIPGFVEGQKNYAKSYRISVHNWSYGRLIESINTQSAKVGIVIETGQQPTKGSPQEQARDLALFAYQYRIA

SEQ ID NO: 273 - protein, 640 aa, unidentified
  source 1..640: note = MG64-66 effector
MSVITIQCRLVADESTLRHLWELMAEKNTPLVNELLERLAKHSDFEAWLEDSKIPKTAIKELCDSLKTQESFAGQPGRFYTSATTLVAYIYKSWLALQKRRQRKIEGKERWLEMLKSDAELEQESNSSLEIIRTKATQLLCFFTAPHNSDTIQKSKAKKSKKTKENNAGSSFSIKSGVLFETYRKSEDVLTKCALVYLIKNNCHLSFLEEDPDKYVKLRRKKEIEIERLKDQLKSRVPKGRDLTKEKWLETLEKAVNSVPQDENEAKSWQASLLRKSSSVPFPVTYETNEDMHWEVNDQDRIFVSFSGLGKLKFEVYCDQRHLHWFQRFVEDQETKRKSKNKHSSSLFTLRLGRLSWLKQEEKGEPWHVNRLILFCSVDTRMWTVEGTQQVAIEKIADVEQNLIKAKEKGELNSNQQAFVTRQQSTLARINTPFPRPNKPLYEGKSHILVGVSLGLKNPANVAVFDAANNKVLAYRSVKQLLGDNYHLLNRQRQQKQRLSHERHKAQKVFAPNDFGESELGLYLDRLLAKEIIAIALMYSAGSIVLPKLGDMREIIHSEVQARAEKKIPGFKEGQQKYAKEYRKQVHNWSYGQLIENIQSQAAKVGILIETGQQSIRGSPQEQARDLALFAYQCRIASSI

SEQ ID NO: 274 - protein, 668 aa, unidentified
  source 1..668: note = MG64-67 effector
MNLRLKAIALLRSWLHNQSGLLLKIFNSMSVITIQCRLVADEKTLRHLWELMAEKNTPLVNELLDRLGKHTDFEAWVQAGKVPKTTIKAMCDSLKTQEPFIGQPGRFYTSATDLLAYICKSWLVLQKRRQRKIEGKERWLEMLKSDVKLEQESNSSLELIRTIATEILNKFSASSTDGINQKSKGNKSKKFKKDKADEPISIKPGVLFEAYQKTEDILRRSALVYLIKNNCQVSLIKEDPDKYAKMRRKKEIEIERLKEQLKSRVPKGRDLTGKKWLETLDKAANSIPQDENEAKSWQASLLRKSSTVPFPVTYETNEDMYWEINDKGRIFVGFNGLSKLKFEVYCDQRHLPWFQRFVEDQETKRKGKNQHSSGLFTLRSGRLSWFKQEGKGEPWSVNRLILFCSVDTRMWTVEGTQQVAIEKIADVEQNLTKAKEKGELNSNQQAFVTRQQSTLARINTPFPRPSKALYEGKSYILVGVSLGLENPATVAVFDAANNKVLAYRSVKQLLGNNYNLLNRQQQQKQRLSHDRHKAQKQFALNDFGESELGQYVDRLLAKEIVAIALTYFAGSIVLPKLGDMREIIQSEVQARAEKKIPGFKEGQQKYAKDYKRSIHNWSYGRLIENIQSQAAKPGILIETGQQSIRGSPQEQARDLALFAYQSRIASSI

SEQ ID NO: 275 - protein, 640 aa, unidentified
  source 1..640: note = MG64-68 effector
MSVITIQCRLVADDKTLRHLWELMAEKNTPLVNELLDRLGKHTDFEAWVQAGKVPKTTIKALCDSLKTQEPFIGQPGRFYTSATILVAYIYKSWLALHKRRQRKIEGKERWLEMLKSDVELEQESHSSLELIRTRATEILSKFSASSTDGINQKSKGKKSKKVKKDKADEPISIKPGVLFEAYQKTEDILRRSALVYLIKNNCQVNLAEEDPDKYAKMRRKKEIEIERLKEQLKSRVPKGRDLTGKKWLETLEKAVNSIPQDENEAKSWQASLLRKPSTVPFPVAYETNEDMHWEISDKGRIFVSFNGLSKLKFEVYCDQRHLPWFQRFVEDQETKRKGKNQHSSGLFTLRSGRLSWLKQEGKGEPWSVNRLILFCSVDTRMWTVEGTQQVAIEKIGDVEQSLTKAKEKGELNSNQQAFVTRQQSTLAKINTPFPRPSKPLYEGKSHILVGVSLGLENPATVAVFDAGNNKVLAYRSVKQLLGNNYNLLNRQQQQKQRLSHDRHKAQKDFTRNDFGESELGQHIDRLLAKEIVAIAVTYFAGSIVLPKLGDMREIIQSEVQARAEKKIPGFKEGQQKYAKEYRKQVHNWSYGRLIENIQSQAAKVGILIETGQQPIRGSPQEQARDLALFAYQCRIASSI

SEQ ID NO: 276 - protein, 866 aa, unidentified
  source 1..866: note = MG64-69 effector
MSSSEKPIPFEKAMQTILALLTIDQDSRQYLWNMCIAYTLLINEIFQRVAQHPKFLEWLNQGKLPIGIVKLICKKLEEDEFTGLPKRVYVSAILLVSHTFKAYFAMQSDLQLKLKGKQRWLEVMETDLELAKNTDITSDLIRVRAAEILTEIEAQRSLSSNQQDRQSEPQEQNNSTSLASNSLMSFLFKKWDIAESSLERRAIAHLLRNDCQVNSEEEDPDKLSLRLERKEIEIQRLEDRLKSRLPKGRDPLGERSQQFLEEAIAFAEHYSYVIQNWFWLKWHQTILSRHPADAERLNYWTLFLIYYRWNNAAEFKAWEQDLSRRAANLQTSFSSLPYPLLFESTDDLYWSWEKEEIKDQTRAQNQSTSKNCKKDLKQKRHRTRKRKRQLEKRICVSFKSKGLKCFRFKLYCDRRQLPVFRQFVTDFETYRALPKEDKFSIGLFALRSAHLLWKEDKQGLERKKHWRLQNLWLKWYCAMLHNSSLEGEIMDSWCRSLIYLEISIRLPWKTHRLHLQCTFDPRLLLAEGTEAVRQEKLTLVRKKLDNLEKSLEISEKQIQSCQNSEAQSIDNLTESEELAEPPSEEEKNQKLINRQKARIRILSTLDRLENSTPTRPSMISYNGNCDIVASVCFSRLNVIGIAVINTRSQTVLAYQNLRTLLTNQRTEVLERRAVKITSRKGRTIVKHSAEHPAKFKARRKIKVQRKARRSVVQLSLEQYRLFNRWQNEQRKNLSGRKEEQKRGLYAESRKESNQAQYLNRLIARRLIQLCQKWQVGSIILPDFGDLRESVECEIQARAKRKFPDDNVKLQKQYAKHLRMAFHRWNHKGLSQAICSCAASMGIPVKTGQQPSQGTLREKALAMAIAV

SEQ ID NO: 277 - protein, 705 aa, unidentified
  source 1..705: note = MG64-70 effector
MVVSMFTIQSRLCASEETRRYFWELMEKHTLLVNELLEKIAQHPQFQEWQKKGAISGNTVRGILAPLKENSGYVGLPGRFYTSAELISCYTYKSWLALQKERQLQLLGKKRWLQAVESELELIATTDFNPDEIRVKAHEIQKKALDKLNKESKKQKALISILLDMHDGTAEAPLSRRAINHLLINNLKINEKEQNLDQLSERLDKKRKEIERLEEQLTSRLPKGRDPTEQRYLENLCHVTALPELSDDPEKLAAELETLTVQKQLPLLKELPYPIQFGSSGDLYWSVETQEKKHCQRPQQRICVRFKGAKDHTFKIQCDCRQLSIFRQFLIDYQTYQELPYEERFSQGLFALRSACLIWRKDDSKHGSNKKRTTDNQEAQLKPWNTHRLYLHCTVDRQMLTAEGTEQVREAKKKEVIKTLKNKEKLQELELEQLGLTKTQIESVGRKRSTLTYLEKNSPPPRPNAKPYQGQPHIVVGVSFSRHEPVAIAVVDVEKEEVLERQSAKELLNRGEAQYIWRNGKKEPLIKDGTEQRHPNGGKLYIRKGKRVRRKPHRLVQQLHQRHQQNSRRRSEEQKQDRYRSSNSDSDLGLYVERLIASKIVELALQRKAGTIAIPQLKGIRESVESDIRARAERLFPNEKERQKEYGKGYRASFHSWSYSRLSDCIKECASSEGIAVVIRQQPSGIELEQKAIAIALSSYNVKTS

SEQ ID NO: 278 - protein, 641 aa, unidentified
  source 1..641: note = MG64-71 effector
MSIITIQCRLVANESTRRYLWQIMAEKNTPLVNELLKQVGKHPDFEKWLQAGKLPPGVVRPLCNSLVSQECFANQPKRFYKSAIELTEYIYKSWLALQQRRQKQVDGKTRWLSMLKSDAELVEISGCSLENIRAKALEILVRITTYSSNQQQQKKKSKKVSNSKASSKLSTTLFETYDQTEDKLERCAITYLLKNNCQVTEEEEDEKKFALRRRKKEKEIERLKEQLASRMPHGRDLTGEQWLQTLLIATTTVPESEEEARSWQSSLLKKPSSIPFPVEFTSKEDLVWSKNQNGRICVKFSGLGEHIFEVYCDRRQLHWFQRFLEDQQIKRSGKDHYSSGLFTLCSGCLAWQENEGRGVRGSAATAALWNVHRLTLYCSLDTRLWTIEGTETVSQDKAAEVAKELTKMQEKGDLNPNQQNYVKRLSSTLTKINNPYPRPSKPLYQGQTSILVGVSLGLEKPATAAVVDASTNTVLAYRSLKQLLSENYKLLNRQRQQQQRNAHERHKAQKRSTPNQLSESELGKHLDNLLAKSIITLAQTYQAGSIVVPNIKNVREVIHSEIEAKAENKCPNFKEGQQKYAKQYRQNIHRWSYSRLIDCIHTQAAKAKIPLEQGPQPIRGSPQEKARSLAIAAYHSRQNKS

SEQ ID NO: 279 - protein, 704 aa, unidentified
  source 1..704: note = MG64-72 effector
MSKSTFLFRLDALGDASQIREENRRYYWELMTQQHTPLINQLNLKVAQHPNFADWQINNAVNADELKSLWRSFKEHPQFETMPERAFVSARLVVGDTYESWLALQGDRQEELAKLSNSLEVLKTDAELVAISGCSLDAICSKAREILSQANTQVAGESKKKSKKAINGGIYKVLYEKHRIADDPLKKCAAAYLIKNRFEIKGDETEEDIEHLKNRIHCKEKQIELLLKQLQSRLPKGRPWVGDGILDEIKSIGVVEDEAEWNLVESALLKQQTFLPHPMLFHSSDDLIWFEHERPPSQDSTTEDERKPNAESSRRVCVRFKSFDEKYAFEISGDRRHLHILQQALRERMIYDSDIDGNTSKLFLVRSATLIWKEYRKNENRIIRRRKAANKRAKRIVQSIDKAPEIQSAPAFYNPEFPWNRYQLFLHCTVETEFLSQEGTELVIQQQRKPIIKALQTLEERMAESENKGESTQTRKANHSRKTGTLRRIDSYDNNYDRPSKPLYAGIPHILTGVALGSGGLVTVTIVDATSGKILGCRGLKALLGDNYRLVNRRQFQRQLNLRRRTERQKRGASSQSGESNLGDAIDQHTANAVLDFAKKHHSGCIVLPDMKDYRVRQQSEIAALAERECSGWKGIEKRFAKAQNMKIHAWSYGRLMEYIRNQAHKGGILVKIGQQPLYGSSQEQAGKMAIDAYQNKTVPKNSP

SEQ ID NO: 280 - protein, 612 aa, unidentified
  source 1..612: note = MG64-73 effector
MSQITVQCRLVAPEPVRQTLWELMANLNTPFINELLQQTAQHPDFGQWRQQGRLKATIIKQLGNQLKEDSRYLGQPGRFYTSGISLVEYVFKSWLKLQQRLQQKLYRKRRWLEVLKSDEELIIETGVNLDVIRNKATKIIQAHQSEDKLFNTLFDAYKEEQDLLTRNALRYLLKNRCQLPKAEEDAQKFARLRRKTEISTERLQDQLDSRLPQGRDLSNNTWLETLAIARNTDPKDQKEARSWQDKLLTQASSIPFPVTYETNEDLTWSKSKKGRLCVQFSGLSDLIFQIYCDQRQLKWFQRFYEDQQVKKNGKDQHSSALFTLRSGRILWQEGTGKGHPWNIHRLTLQCTLDTRLWTAEGTEQIKQEKAEEIAKVLTRMNEKGELNKNQQAFIKRQQTTLGRLGNAFPRPSKPLYQGQSNIIAGVSMGLEKPATVAIIDVITGKTLTIRNTKQLLGKNYFLLNRQRILKQSQSHQRDVAQRKEAFNRFGDSQLGEYIDRLLAGAIVQLAKTYQAGSIAIPKLEDIRESVQAEIQARAEEKIPNCLEAQAKYAKQYRVSVHQWSYGRLIENIQAQADKVGIVIEEAEQVIRASPQQQARELAINAYAARSLA

SEQ ID NO: 281 - protein, 789 aa, unidentified
  source 1..789: note = MG64-74 effector
MTMKAIQFDVIARKDPKYRRNREKPTDQLEEEALWEVVQASCHHTLLVIEILKQMEQPSAFPARIKKLKQPQADGILPDIKQEEEWLETEIEKACKSLKEQAEFQNLPGRIYSSAIHQSLQPLKGWLENQWQLLLRLSGKNRFLAVVETDADLAQASDFSWSDIQARAQAILQQTQEAIAAKAKDETAAKDTKQLLKSLLKQYDATSNILARRAIIHLLRNKFKVRRKPENPKRLQALLEGKRVEIERLEAQVPRLPRLRNLVPEQAYDTALEELTTYPLSDVAVSERVAWLLHYRVLICFFLIYITSAEKNLQLADCLLHLVRIEIERGEAQFYQWHDGVPAKINQFLTIPKSLPYPIYFGGDNLRSWQLNQEGKICFKLNGLGDYLFEVRCDRRQLGIVKYFLQDWQTQNKNKNEYSGGLTLLRSAELLVKPKLGKQNAKLPPIHDRQAVVTAYKLSLHCTYDTDYLTHQGLECVRQRKIANQLKGLTDKKAKLTKQQEQLQQLEQEMQQEQIGTSAKRSKRHAQRLKQIEQLKQSISKLQAAIQAELERPRPKLERLQQSQLFQRADRPLYAGVAHLFVGVCLDLDQHLVVTIVDAMRHKVLSKRTGKQIMGEHYPLLQRYRRLKQQHPKQRRQDQKVGRHNHLSETGLGEQVACAIANGLLSLAQQYKVSTIVLPETKGWRERLYSQLVARAKIKCNGSKKAMARYTKAYGKRLHQWDYNRLSRAIETEAQTVGVTVIFQRLEFQANAEQDNQPADEADEQDNQRVNPFELALQIAIAAYDSLQA

SEQ ID NO: 282 - protein, 642 aa, unidentified
  source 1..642: note = MG64-75 effector
MSIITIHCRLVASEPIRRHLWHLMTESNTPLINDLLNQVSQHPDFETWQRRGTVPEKTVKELCEPLKAIYPGQPARFYASAILMVTYTYESWLALQQNRRRRLDGKQKWLNVVKSDAALLELSSTTLEAIQERAQTVLKQLNVEPETQAASNPKKRKAAQQQTQSANKASPMTLLFEAYDAIDETLSRCAIAYLIKNGCKIPETEEDPEKFAHRLRRKQKEIEQLEAQLQARLPKGRDLTGEEFMETLAIATQQISESVAQAREWQAKLLSRSVCLPYPIIYGSSTDVRWGTTAKGRIAVSFNGIDKYLKATDPDIEAWFKTSQEPPFRLYCDQRQLPFFQRFLRDWQAYQAEKDTYPAGLLTLSSAMLAWREGEGKGDPWNVNHLALYCSFDTRLMTAEGTLEVQREKADKALKNLTHAKPDPRNQSTLNRLKNLPDRPSKKPYQGKPEILVGLSIGLANPVTVAVVNGTTGDALIYRTPHTLLGNHYHLFNRHRQQQQQNALQRHKNQRRGVAYQPSESELGQYVDRLLAKAIIQLAQTYQAGSIVVPNLTHLRELLASEITARAEQKASLVEAQNKYAKEYRQTIHRWSYNRLIEAIRSKAQQLGITVESGFQPLQGNSQEQAKDMAIAAYHARAINTK

SEQ ID NO: 283 - protein, 640 aa, unidentified
  source 1..640: note = MG64-76 effector
MTVITIQCRLVAKEETLRHLWELMTQKNTPLINEVLEQIGKHPELEECIQKGKLPIGLVKTLCNSLKTDLQFSGQPGRFYSSAISLVDYIYKSWLALQQGRQRKIEAKERWLSILISDDELEKACNSSLDVIRAKATKLLTQNGPPSNLNHNQPTESKKGQKTKKGKADKPPRRLFNLLLDAYENTTDPLERCSLAYLLKNDCQVSELEEDPTEFALRRRAKEIEIERLKEQLESRLPKGRDLTGEKWLEALETAIHNISKDEDEAKAWEAALLRKSSSVPFPVAYESNEDMNWFKNDQGRICVRFNGLGKHTFEVWCDQRQLHLFQRFLEDQQTKRDSKDQHSSSLFTLRAGRICWLERQGKGLMWNRHRLILYCCVDTRLLTAQGTQQVKSEKAAKIAKILTKTKDKEELDDKQQAFVKRQQSTLDRINTPFRRPSKPLYQGQPSILIGVSLGLKKPATVAVVDAKEGKVLTYRSVKQLLGENYKLLNRQRQQQQSLSHERHKAQKRDTPNEFGESELGQYVDRLLAHEIVAIAKAYQAGSIVLPRLGDMREIVSSEVQARAEQKVPGYKEGQQKYAKQYRVSVHRWSYGRLIEIIQSLAAKTGIAIEVGQQSIRGSPQEKARVLALSAYSSRITCMN

SEQ ID NO: 284 - protein, 742 aa, unidentified
  source 1..742: note = MG64-77 effector
MNMLMFTIQCRLCASEQTRRYWWESMEKYTLLVNELLEKIAQHPQFQEWQKKGDISREAVRKILNPLKESLQYAGLPKRFYTSAELISCYTYQSWLALQQQRQLRLLGKQRWSEAVESEFELSATTDFSPDKIRVKAHTILNKAIQKLNQQGKKPKHLMDILLKKHQKTAKDSLSRRAINHLLINNLKVSQEEQNLNELSERLDKKRVEIKRLEEQLKSRLPKGRDPTRQRYLQILSHISVLPDLRDDPQKLEAELDRLTIQQQLPLFNELPYPILFYSSSDLYWSVQPQDTSHPSEPENGSHPELPKSKKHQKPHGKRPTERISVKFKGVQSKEAKPNTGQSKEAKDHTFKIQCDRRQLPLFRRFLIDYQTYDNLPEEERFSEGLFTLRSACLIWRKDESRHRSTKKIGTDQPEDQLKPWNTHHLYLHCTVDRRLLTAEGTEQVRAEKKQATLKELKGKDKLEQTELDELGLTKNQISSIKRKCSTLNRLENHSLPPRPNSVPYQGQPGITVGVSFSRHQPVAIAVVDVNKQEVLERQSAKELLNRGKAQYLWRNGLKESLTRDGTERRHPNGGKLLIRKGKRVRWKPYRLVEQLHRRHQHHSQQRAKQQQQNRYQESNVDSNLGLYVDRLIASRIVKLALQRKVGTIVIPQLKGIRESVESDIRAQAERKFPNEKERQKEYAKHYRASFHSWSYARLAKCIKECGAREGIAVVERKHSSQGDLEQKAIAIALSSYNVKTS

SEQ ID NO: 285 - protein, 515 aa, unidentified
  source 1..515: note = MG64-78 effector
GRDPTRQRYLQILDHISVLPELQDDPQKLEAELDRLTQLPLYNELPYPILFHSSGDLYWSIESQDTSNPCSPENGIHPELPKIKKRHKKHCKQPTERICVQFKGTESEEAKPKKSQSEVAKDYTFKIQCERRQLPVFRQFLIDYQTHKQLPEEERFSEGLFALRSACLIWRKDDKRHRSKKTRTADQPEESPKPWHTHRLYLHCTVGRPLITAEGTEQVRQEKKREVIEELQGRDQLEESQLQELGLNKKQIVYVKRRRSTLNRLKNNSPPRPSIQPYQGQPHIVIGVSFSRHQPVAIAVVDVNKEEVLECQSAKELLNRGEAQYLWRNGKKELLIRDGTERRHPNGGKLYIRKGKRIRWKPYRLVEQLHQRYQHHSRQRAKQQQQNRYQQSDSDSNLGLYVDRLIAAQIVELALQRKAGTIVIPQLRGIGESVESDIRAQAQRLFPNEKERQKKYALHYRASFHCWSYARLSQCIRECATREGIAVVERKQSSQGDLEQKAIAIALSLCNVKSS

SEQ ID NO: 286 - protein, 638 aa, unidentified
  source 1..638: note = MG64-79 effector
MSQITIQCRLVASEPTRQHLWKLMADTNTPLINELLKQVGQHPDFESWRHKGKLPAGIVKQLCQPLRTDPRFIGQPGRFYMSAITVVDYIYKAWLALQKRLQYQLEGKTRWLEMLKSDTELIEATGCTLDILRIKATEILAQHAAFAPDPTQTPTTKGKKGKKRRTANTNHNLSEALFEAYRETDDILTRACICYLLKNGCKVTTKEEDPEKFNLRRRKVEIRVKRLTEQLASRMPKGRDLTSETWLETLAIATSHVPQNEDEAKSWQASLLRQSSSVPFPVAFETNEDLRWSKNQKGRLCVEFNGLSEHTFEVYCDKRQLHWFQRFLEDSLIKRDSKNQHSSSLFTLRSGRIAWQEGEGKGDPWNVHRLTLYCSIDTRLWTHEGTEQVRDEKAAEIAKTLTAMKEKGDLNEKQQAFIKRKNSTLARINNSFPRPSNPLYQGQSNVLVGVSLGLEKPATAAVVDAMTGKVLTYRSIRQLLGENYKLLNRQRQEQHQNSSKRHNAQSQGAPNQFGESNLGEYVDRLLAKAIIALAKTYHAGSIVLPKLKDVRESIQSEVQARAEQKCPELIEAQKNYAKQYRSSAHSWSYGRLIESIQSQAAQAGIIVEEARQALVGSPQDKAKKLAIVAYTSRLQAII

SEQ ID NO: 287 - protein, 510 aa, unidentified
  source 1..510: note = MG64-80 effector
MSEITIQCRLMTSEETRRYLWQLTAPKNTPLVNELLKLVSQHPDFEAWRRRGTLPGNAVKQLCEPLRQDPRFVGQPGRFYSSAIRIVQQTYKAWIASIRAKQASLDGKKRWVETVESDAQLAEMGNFSPEVIYTKAREILEQVSTVLLSPTTPAKQPKRAKKSKKNKENSSVINTLFELFNTTEDLLSRRAIIHLLRHGWQVNEQEEDPEKLSQLLARKRQEIKRLEEQLQARCPRGRACTEEEAQERVRRAISLPEHPGLMFSLYLSVALLCAPTPSSRQLILTWLLKRICQFEEARVHSEFLDWEENFPSNRLSLMRSPKNLPYPILYGPEDLNKWCRNEKGRICLSFNGLSEYTFELQCDRRQLSLFELFMEDWQTLRAKENQKQYSGSKLLLREATLFWQEPTQKIIKKKFNRQPDTQTEHPLENEATQQRCGRNSDPWNKYRLTLHCTIDTKLLTLEGTEQIRQEKLAKRSKELESRSQKSKLDEDQALNRERAKQECLQNAKYS

SEQ ID NO: 288 - protein, 746 aa, unidentified
  source 1..746: note = MG64-81 effector
MFTIQCRLCTSEETRRDIWQWMEKYTLLVNELLEKIAQHPQFPKWHKKGNITRKAVGEILNPLKENPQYAGLPSRFYTSAELISCDTYKSWLALQQQRQLRLLGKQRWLEAVETELELSATTDFDPDKIRAKAHSIREEALQKLNQQGKKPKDLMDILFKKHQKTVKDSLSRRAINHLLINNLKVSQEEQNLNELSERLDKKRVEIKRLSEQLKSRLPKGRDPTRQRYLQILDHISVLPELQDDPQKLEAELDRLTQLPLYNELPYPILFHSSGDLYWSIQSQDTSNPSSPENGIHPELPKIKKRHKKHCKQPKERICVQFKGTDSEEAKPKKSQSEGAKDYTFKIQCDRRQLPVFRQFLIDYQTHKQLPEEERFSEGLFALRSACLIWRKDDNRHCSKKKRTADQQEESPKPWHTHRLYLHCTVGRPLITAEGTEQVREEKKREAIEELQGRDQLEESQLQELGLNKKQIVYVKRRRSTLNRLKNNSPPRPSIQPYQGQPHIAIGVSFSRHEPVAIAVVDVNKEEVLECQSAKELLNRGEAQYISRKGSKELLIRDGTERRHPNGGKLYIRKGKRIRWKPYRLVEQLHRRHQHHSRQRAKQQQQNRYQQSDSDSNLGLYVDRLIAAQIVNLALQRKAGTIAIPQLRGIGESVESDIRAQAERLFPNEKERQKKYALHYRASFHRWSYGRLSQCIRECATREGIAVVEKKQSSQGDLEQKAIAIAISLCNVKSSWLKHSSICTLTR

SEQ ID NO: 289 - protein, 530 aa, unidentified
  source 1..530: note = MG64-82 effector
MFNAYYATDDTLQKCAIAYLIKNKRQVNDKEEDLKKLTRLIKQKKKKIERLQKQLESRLPKGRQWLGNDFIDNLKAFGIPESEVEWFSLQSALLGEHNFIPYPILFGSSDDLIWSKQLKISNNSNLIKEKLESEKSRERICVQFKGLKEVVFEISCDRRQLPLFQQFLKDWTIYSQNPKEHTSSLFLIRSATLIWKDTKKTKNRQKRWNKNKTVNQKCIDPQQEELKQLVQAGINEEEQPWNRYQLFLHCTVATEFLSKEGTQQLGQKKQELALKAIATLEQKILELEKEGKSTKNDRESFSRKQGTVRRLNNLDNPFERPSRPLYQAQPNILLGVSLGSSKLATATVVDVTTEKVLECQGVRCLLGDNYKLLTRKQYLHEMHSHLRSKAQKRGAKNLLREAKLGEHIDRLIATAIIALARKYQASTIVLPDMKDYTEKKQSEIEAFAEQECSGWKCVEKRFTKAQSVKLHRWSYSRLSKIICQQASKVGIAVEIGQQPIHGSSQEQSRAMAIETYHSRKNSLKSKNLRS

SEQ ID NO: 290 - protein, 690 aa, unidentified
  source 1..690: note = MG64-83 effector
MSTITIQCRLVASEPTRQQLWTLMAERNTPLINELLAQISQHPDFDTWRQQAKLKPVIVKQLCQPLKSDPRFSGQPGRFYDSAIALVEYIYKSWLKIQQRLQRKLEGQSRWLEMLKSDEELVQMSNCTLEVIRAKAALVLTPLASQNQSTQPTNTKSKKRKKPQASNSNRSVSKALFEAYANTEDILTKSALCYLLKNGCKISDKEEDPEKFAKRCRQTEIKISRLTEQIASRIPKGRDLTGEKWLETLITATSTAPESETQARSWQDRLLTQSKSIPFPVAYLSNVGLTWSKKEKEENGKNLSKYRKGGRKNQEGRLCVKFNGLGEHIFEIYCDQRQFQWFQRFYEDGQIKKESKDQHSSALFTLRSAQIVWQEGFGKGEPWNIHRLALYCTLDTRFWTTEGTEQIRQEKIITLEKTLLRIKPELTLELFFRSHLILKFLSIWCVITTHKTVEMLKEGDLSEQRKNSRAFRKSTESSLQKINTPFPRPSQPLYQGQPHILVSVALGLDKPATAAVIDGTTGKAIAYRSIKQLLGDNYKLLNRQRQQKRSYSHQRHKAQKKAASNQVGGSDLGQYLDRLLAQAIVKLAQTHQAGSIVLPKLGDMREVVQSEIQARAEQKIPGYIEAQEKYAKQYRVNIHHWSYGRLMDNIKAQSSKVGIFIEEGEQPIRGSPQEKAKNMGISAYHARSNS

SEQ ID NO: 291 - protein, 846 aa, unidentified
  source 1..846: note = MG64-84 effector
MSDVLLVSDRDTEGELKAMRTIRLVLLADAETRQHFWCLSLVHTFLVNELFQTLPKHKDFPKWQRQRRVSVESVKKLIDQLKKKDFLGTPPQIFSSAIAIVCSTFKAYFALQQKCQLKLDGMRRWLRTVESDLELARTTAFSAETICSRARALLVELEAQRHHNRHEPQLQASHEPQPLPAPTSLMSDLFKRLESAEDPLERRAVVHLLKNACAVNVAEEDPDKLALRLEKKKIQIQRLEKQMASRLPVGRDPTGDRAHQAIEAAISFAEHTPVSFWLKWHGVLLTNRSVNALSLELWVLGYLYDCLNADAEFEAWEQALPSRMANLSTQWAALPYPLIFDSTDDLYWSRAPEPPLKPPCSGKKAAGTMKPLHHKRKRSRTRKRQKKLTERIMVRFKGKGLSHCRFKVGVDRRQLPIVQQMVDDEQAHKARAADDKFSLGLFALRSACLRWDVDPQKLHTKQHWKLQSLWLKWFCTLPNGTLMQQSELDLWFISLFYLALSKSIPWQTHRLSLHCTIDPRLLTAEGTEAVRQEKLAQTLERIAQVEKKLQLAEQQTEQQAHEGGETLATAGEITQLDEDLDEESVAQKQESVAQKRQDRQGAIKRLHSTLTRLGNASPSRPPRQPFAPQRDIAIGVCFSRKNVLGVAVVDTRSQAVLEFCNLRSLLTDDRLALLTKRAAKIPASRKGKRSVRQFQLKDYRLFNRWRRLRHQNLTQRGDQQRHGLYAESSQESNLAQHLNRVIAKKLLQLAQQWQASRLTLPDFGNLRESVECEMQARARRKFPDDNVKLQKQYAKELRMFHHRWNHKQLAQCLRACAARTGVPVITGTQPKEGELRDKAVALALAG

SEQ ID NO: 292 - protein, 641 aa, unidentified
  source 1..641: note = MG64-85 effector
MSIITIHCRLVASEPIRRHLWHLMTESNTPLINDLLNQVSQHPDFETWQRRGTVPEKTVKELCEPLKASYPGQPARFYASAILMVTYTYESWLALQQNRRRRLDGKQKWLHVVKSDAALLELSGTTLKAIQQQAQTILNQIDVGPETQGLPNAKRRKPAQKQAKSASTASLMTRLFEAYEATDEILSRCAIAYVIKNGCKIPETEEDPENFAHRLRRKQKEIEQLEAQLQARLPKGRDLTGEEFLETLAIATQQISESVAQAREWQAKLLTRPASLPYPIIYGSSTDVRWGNTAKRITVNFSGIDKYLKANDPDLAAWFKTTKASPFQVYCDQRQLTFFQRFLDDWQTYQANKDTYPAGLLTLSSAMLAWREGKGKGEPWHVNHLALYCSFDTRLMTAEGTLEVQQEKAAKALKNLAHANPDPRNQSTLNRLQHLPDRPSQKLYQGKPDILVGLSIGLANPVTAAVVNASKGNLLTYCTPRTLLGDHYHLLNRHRQHQQQNVLQRHKNQQRGVAYQPSESELGQYVDCLLAKAIIQLAQAYKAGSIVIPNLTHLRELLASEITARAEQKASLVEAQDKYAKEYRQTIHRWSYNRLIEAIRSKAQQLGITVESGFQPLQGNPQEQAKDVAIAAYHARAINAK

SEQ ID NO: 293 - protein, 637 aa, unidentified
  source 1..637: note = MG64-86 effector
MSHITIQCRLVASLPTRRQLWELMADKNTPLINELLALVANHPDFETWRQKGKLPSGTVKQLCQPLKTDPRFISQPARFYTSAIKVVDYIYKSWLALMKRLQYQLEGKTRWLEMLKSDAELVESSGVTLETLRSKATEILAQLTPESDSVASQPPKAKSKKKKKSKALDSKPNVSHILFDAYRNTADILNLCAISYLLKNGCKINDKEEDQNKFSQRRRKVEIQIQRLTEKLTARIPKGRDLTNTRWLETLAEATSCVPQNEAQAKYWQDNLLKGFSLVPFPIIYETNEDMTWFKNVSSRLCVKFSGLGEHTFQVYCDQRHLHWFQRFLEDQEIKKNSKDQHSSGLFTLRSSSMAWQEGEGKGEPWNLHHLTLYCCVDTRLWTAEGTKQVKEEKATEIAKILTKAKEKGDLNQQQQSFIQRKNSTLTRINNPFPRPSQPLYQGQGNILVGVSLGLEKPATVAVVDAIAHKVITYRSIRQLLGENYKLLNRQRQAQRSSSHERQNAQRRDAFNQLGESELGEYIDRLLAKEIVAIAQKYQAGSIVLPKLGDMREIVQSEIQALAEQKCPEFLEGQQKYAKQYRVSVHQWSYARLIDCIQTQAKKLGIAIEEGQQPVRGSPQDRAKELAIAAYHLRSKA

SEQ ID NO: 294 - protein, 725 aa, unidentified
  source 1..725: note = MG64-87 effector
MSMFTIQCRLCANEETRRSFWKWMEKYTLLMNELLENIAQNPQFPEWQKKDNISRVEVREILKPLKESSRYEGLPGRFYTSAELISCGIYKSWLALNKRRKLQAIGKERWIKAVESEFELSATTEFNSDEIRSEAHRILEKETQELKKKERNPKELIAILLNRHEKTEHSLSRRAINHLLINNLQINEEELNLDKLSERLDKKKVEVRRLKEQLISQLPKGRDSTRQQYLQILDHISVSVLPELSDDPQKLEAELDKLTIQQQLPLFNELPYPIRFDSSRDLYWSIQSQSPSNPSENGSHQQLPKNEKPQTKHDQRAKDRICVEFKGTKDYIFKIQCDRRQLPLFQQFLTDYQTYTQLPEEERFSEALFALRSARLIWRKDDDSRHSSKKKRTTDKQEDQLKPWNTHRLYLHCTVDRRRLTAEGTEQVREEKKREVIKKLKGRDRLEEAQLQELGLTKNQISDVKRKRSTLNRLKNYSPPPRPRVPLYQGQPHIVVGVSFSRHQPVAIAVVDVAKAEVLECQSAKELLNRGEAQYIWRNGQKEPLSKNGSERRHPNGGKLLIRKRKRVRFKPYRLVEQLHRRHQQHPPRRAEQQKQNCYTNNNSDSNLGLYVDRLIAAKIVELALKRKAGTIAIPQLEGIRESVESDIRAQAERLFPNEKERQKEYAKHYRASFHRWSYARLSECIRECAKREGIAVVESKQSSQGDLEQKAIAIALSSYNVKTS

SEQ ID NO: 295 - protein, 696 aa, unidentified
  source 1..696: note = MG64-88 effector
MVVRTIRCRLTASRETRQFFWEKMVAYTCLINQLFSKVAQDEQFNDWQQSSSVPRKPLEEIIKTIEKESGSYHLPARFYTSAVLMTQYVYKSWFALQKRRQWQIQGKRRWLEIMKQDSLLAHTDFSPETIYAKAREILSQTSNNRNEPKKKRSEGKKSLLGSLMTKFEETDDLLTKCAVLHLLKNDFEVSEETHDDPDDFKLRLESKRIEIERLEEQLQSRLPKGRDPTGDRFAENLIEAIALPDDTVSNYSDLVFSSWLEQKQIKLLNPLPYPIIWGSADDLRWTSEPRKLPPNAPRASSNTKKKKKPAKKKQITSEDIIGVRFKGLSAHTFKVQCDRRQLPIFRQFFTDYKAYNALPEEERFSQKIFALCSAQLIWCQDSSKSKQKKPKDNPSKESWDSHRLYLHCTIDTQYLTAEGTADAIRLAKQKILKELGERATIPPEEIDSLDLTQPQKSHIKRKRTTLKRLDNPSPIRPRREEYQGNPLITVGISLSRQMPLTACVVDIRTSKVLECQATKRLLLIKKFKIKSKKHNAHQLKRAHWRLVNKLNLRKKRNSVQRQSKQKQDAYRESESESNLGAYTERLLANRVVALAMAWEAGSIVVPDLKNIREVAESDIKARAQERFPHEKQLQKQYCKDLRASYHRWSYSRLVGYISDRAALFGISVMTGKQPTHMSLPDSALHVAMSGHQLSAV

SEQ ID NO: 296 - protein, 601 aa, unidentified
  source 1..601: note = MG64-89 effector
MSKLTIQCRLVACEDTRRQVWEMMAGRYAPLIATTLEQVSQHKDFPQWVSAGEIPAQVVKNLVNQAQSGLPARWCASAQRQVQETYKAWLTKRRKLQQKLQGQQTWLSVLRPDAELAQEAGLSLEEMKIRAQALLHREINNWFQVYQQCQDVVERSIFAYLLKHRLTVPTEPEDTDKLRRKRRQVEIKIERLETQLAGRSPQGRDLTGSRYAAALNEGEQCYWENDADFLAWQAEILSRPDSLPPPVEYATNTDMTWHKDEQGRLAVTFNGLGKLKFKIACDQRQLHWFQRFYQDQEQFKSQKGQRSQALFTLRSAELLWKPGNRSGDPWQANFLYLHCTVDSRLWTQEGTAMVQQEKAKKSQAIVKKLSERSDLTAQQKDCLQRHQSTLARLHMGYDRPQRRMYQGKSHLVVGISLDMENLVTVALVDVVKQKVITGCTMKSLLGQDYALVQRLRYEKRQNSHLRKVAQERGSKIVNYEANLAIHVERLLVKAIIHFAQQHLAGSLCVPTLKDIRETIQAHLQCRAEERFPDSKELQRRYAKEYRINAHRWSYNRLLKLLNQQAKFAGLVVEQGVQSAGETALERALGVALSAYYQRSAA

SEQ ID NO: 297 - protein, 634 aa, unidentified
  source 1..634: note = MG64-90 effector
MSQITIQCRLIAKESTRRQLWELMAHKNTPLINELLERIGQHPDFLTWRQNGKLPPGFIKQLGESLKTDPRYAGQPSRFYMSAIALVNYIYKSWFALMKRLQYQLEGKTRWLEMLKSDAELLETSGVTLEILRHQATEILTQLTPQSDSPKPKNTGKKAKKPKASESDRTLSHSLFAAYRQSEDNLTRNAIAYLLKNGCKLTDKEENPEKFAKRRRRVEIQIQRLTEQLTARIPKGRDLTNAQWLETLAIASTTVPETDTQAKSWQDRLLKQFSVVPFPVTYETNEDLTWFKNTSNRLCVKFSGLSEHSFQIYCDQRQILWFERFLEDQQIKRASKNQHSSSLFTLRSGRIAWQEGEGKGEPWNLHRLTLYCTVDTRLWTAEGTEQVKEEKAAEIAKVLTKTKEKGNLNENQQAFIQRKSSTLTRINNPFPRPSKPLYQGQSHIIVGVSLGLEKPATLAVVDAIASKPLTYRSVKQLLGKNYPLLNRQRQEKQRNSQQRHSAQKQSAYNHFGESELGEYVDRLLAKEIVAIAQTYQASSIVLPKLGDMREIVQSEIQALAEQKCPEYLEGQRNYAKQYRVSIHQWSYGRLIECIKSQAAKIGIAIEEGQQPVRGSPQEKAREMAIASYQSRSQV

SEQ ID NO: 298 - protein, 633 aa, unidentified
  source 1..633: note = MG64-91 effector
MTLKTIECRLYAPPETLRHLWELMAKKNTPLINELLHGISEHPDFDKWFKQKKLPQKEIKSLCDRLKTEQAYQNQPGRFYSSAIALTEYIYKSWFAIQKKLQQRIEGKQRWLNLLKSDSELETECGQSLEKIEIEAKNILDRFEKDSSLTKKKKQPKSQSKSEKTLFNYLFDQYNETTDPLNRCVLAYLLKNNCQIPEQDEDLDRYQFRRQKKEIEIKRLQTQRQNRLPKDRDLSGQLWLKILGTVNNCVPQDELEAASWQADLLRKSPVIPFPVSYETNTDLIWSKDGREHFQVRFNGLGKQHQFEIRCDKRQLCWFQRFFEDGEILRCDREQYSSALFTLRSARLLWREGKKDKDNPWEIHTLYLQCSVDTRFWTAEGTKQIASDKSATVQEILNNLKEKAELTPSQLAYQKRQQSTFTRITNPFPRPQKPLYQGDPSIIMGVSLGLEKPATIAIVNVTNNRVLAYRSIKQLLGKNYKLLNHQQRQKQKLSHLRHQAQKTESNNQFGESELGEYIDRLIAKAIVEIAQKYRVSSIVLPHLKQIREITDSELMAKAQRKIPGYKEGQKKYIKQYRCNIHQWSYGRLIESIEQAAAKIGIDIEQIQQSRQGTPQEQAKQLAISAYNSRLQRAI

SEQ ID NO: 299 - protein, 658 aa, unidentified
  source 1..658: note = MG64-92 effector
MGIITIHCHLGTIEPIRRLLWQAMVESNTPLISTLLRQVANHSDFDTWQIKGSVPVKAVRTIGDPLKAHYPPQPGRFYASAYQMVSYTYESWLALQKKTKFSLEGKRRWLSIVKSDAELLELTGLSLESLRQSAREVLSQISAEIAAERVPDTQKNKPKVKSRKSKKKSTGKDKDLIGKLFKVYGTTDDLTQRCILAYLIKNGGTILDQEETTEAFARRVHRKQKEIARLENRLEARLPKGRDLTGDIFTDTLLLAQQQEPEDIAQMRDWQAKLLMRPADLPYPIRYDSSTDMMWKPDDQGRISVNFNGLDKFLKNSDPEVRSWLKEHQGYPFRIQCDQRQLPCFQRFLADWQAYTADAENYPAGLLTLSSAMLAWRKGKKNGKGEPWNIHQLALYCSFDTRLLTAEGTVEVQQEKIKKAQKQAKSADGKKLDEKQLQARTSNATTLRKLDNLPNRPGCKPYQARPELLLGISIGLSEPVTVAIVDAATHQVLTYRTSRTLLGEQHRLLRRQRQKQQQNRLKRQQNQKQGIRHQPSESELGQYVDCLLAKAITQLALTNQVSSIVLPDLLNRRDILDSEIQAKAERKCPGSISAQEKYAKDFRRSLHSWDYRRLIEAIRSSACKHGIPLEETFLTASSDPKEQAKEIAIAAYQARTED

SEQ ID NO: 300 - protein, 698 aa, unidentified
  source 1..698: note = MG64-93 effector
MTKASTIKIYEAKLIPSAWKRPTKKEPTSIATPSEEVKKQVLLLSIRSTQLCKAIGDSLSKRDELDEWISKGAIPEKVLKAEWEIARNQKCFRDMPSRFQTTALLRIQETFSGWFEQRKQKRREIDRSQRWLDIVKSDAELMEISGLDIEDIKAKALEILQEAKKISETSQAQPEPAPESCVSEEKNSADTTPTANPYPNQPYKSVFSILFEMYEEVVQHDLVATCAISHLLKNRASIATETEDTKAFEARIRKKKKEVERLKSQLNSRLPKFRIFSDQLLPYLFDECADLDKAKPPSKAIGKNIPRKHSDLPYSVLFYSRDDITWELIQRMNPNSQVLEDRIFIKIKGLDKYVKQQGIAQPAVFEICCDVRQLSYFKRCHEEWQLYSKNRNDYSTRDFLLQSASLVWKKKNVPVGQRKSSNELDRYEPYLQITIDTDKLTFESSEKKRLVELEAVERIIASYEAKQQDGELTIQQQKGLQRSLTTRRKLESNSFPRPSKPLYQGNQNLVLGVSFGLEKPVAVAIVDLTKEQTITVRSAKQFLGANHNQLSAYRHEQRHNSSQRRKNQRQRKSAEISEHRRGMHIDRVMAKAIVNLAQEYKAGLIVLADCKGIRDRIQSGIEAKAEHKYSKDIERQKAYLKQYRVNVHKWDFRRLSQCIRGKAGKEGIPVEIVKQRYQGDLPAKAAQVAFDGAKVLSS

SEQ ID NO: 301 - protein, 558 aa, unidentified
  source 1..558: note = MG64-94 effector
MLVDRLKLDPRFGEQPVWYYVSAQKQVAYTFRSWLSSQRRKQWRLEGKRRWLEILRPDAELAEIAKCSTEALRSAASRILKEVDDPAPFNFLLKEYGTVNSRKRQCALAYLLKRNAKLDPEVEDLEKLEARRGKTEIQIKRLEMQLQANLPKGRDLTGQIQSEALAQCVQTAFIDDAVYSAWQSTITRKPASLPFPIIYETVESLVWSKDCYGRYSVCFQGQGTSTHTFKIYCDKPHQHWFERFWIDQETKRSGGDQHSAGLFTLRSARLSWVPSKDHQDEPEPWNRYYLNLSCTVDTDLWTQEGTQLVLQKKAASTASKLQAMREKESLNENQQGYVRRLESTMKRLQTPYPRPSRDLYQGRSDILVGVSMGLDKPATVAVINVLNGDVLAYRSTKQLLGEKYPLLQRARSERAKIAHQGHRQRRKGEKNVAQESNLGEYVDRLLAKAIVEVAQQYWAGGIVLPDLSHIREIIEAEVKQKAAAKVPDFVDGQKQYAKAYRAQVHQWSYNRLQNFITSKAEQSGLSVETTKQEYSGSPQEKAKLLCFAGYENRLVLLS

SEQ ID NO: 302 - protein, 649 aa, unidentified
  source 1..649: note = MG64-95 effector
MSQITIQCNLVASEATRQYLWHLMADIYTPFINEMLATIAQHPNFEEWSQSGKIPADVFEDIRKTLKAHPDFQGMPGRWYYAGRDLVKRIFKSWLALRRRLRHQLSGQTHWLEIFQSDDDLVAACGQDLPAIRAEAASILTKIQIEAPNTSKQPKKTKQPKKAGSKTQKPEEEQRNRNLFPALFKEYDGAETELVKCAIACLLKNNCQIPTKAEHPEKFQKRRRKTEIRVERIIEQLARTRLPKGRDLTNEKWLDTLKMAVQQVPKDETEAAAWEADLQTDSSPLPFPIAYESNEDLKWSQNAKGRLCVRFNGLGKHTFEIYCDTRQLHWFKRFLDDQTIKKQGGNSHSAGALTLRSGRISWRLDSSKGNPWDRNRLVLFCSVDTLLWTKEGTEKASQEKASKIAQVISGTKAKGNLTSKQEDFVRKREKTLALLQNPFPRPSRPLYQGSPAILAGVSFGLDKPATLAIVDVTTGKAIAYRSIRQLLGDDHKLLNRQRQRQRQKAQRRRSNQLKFASNRISEGGLGGQIDSLIAKAIVQIAQQYNASSIVLGDLANIREIIESEIQAKAEQKTTLKEIQAKYARDYRASIHRWSYKRLAQKIESNALQAGLIVATIKQPLAGSPQDKARDVAIAGFQSRSVSKILDTGS

SEQ ID NO: 303 - protein, 575 aa, unidentified
  source 1..575: note = MG64-96 effector
MQSEIDLLKTTEFELSEITAAAKLALNSARKQKKKSDKSVEGSSPSLFSILIDIQFKTKSPLKKRGINHLLLNDLNIRQREFELSDLETRLEAKFLEVEDLENRLRSRLPKGRDPDGQRYVSALAEAASISEQYLSPERINEIQASIPIYNELPYPLIYEGGSNISWILLESANSKSKSGRLQVYFSGISELKFSIQCSRRQLPIFRGFYEDKTENKNRFRREEIPFSEGLNRFRSAQIIWKPDSNFQFRKKRGEITSFPWEVNRLHFHCSVNRVTLSAEGTEQLRQAKLKKLTTKDEKSLIPRKRTELERLRNAEPPPRPSVPIHIRDPNIVIGVCFSPDEPVIVVPIDLEIEAALYALNTKALLNQEKKTIWRNGKKETVSDSGELQLHSNGGKLSSRKPGQWFVQKPYDLVTRLNTLTEQEAKLRKREQSKGKYENSESLSNLSLYVCRLIAARLVELSLKLNVSRVILPDLEGIRDWVQAIISAKAAKAFPDSKKQQKQFLQQFRVKYHRWNYRKLCQEIESCARKSGLQVTYARQPNLHEISEQVSAFSRDEEFYTFKVAAEKMALPQEL

SEQ ID NO: 304 - protein, 667 aa, unidentified
  source 1..667: note = MG64-97 effector
MHSILLNIYGIIEHNVFLNLKSMSQNTIQGRLVASVVTRQQLWKLMADKNTPLINELVLQVAQDPDFETWREKGKIPTGIVKQLCAVLKTDSRFIGQPGRFYTSAINRVNYIYKSLLALMKKLQYQLDGKNRWLEMLKSDIELVEASGVNLESLRLKAAEILAQVTPQSDMVEPQPAKGKKRQKTKKSKDSDSDCAERTLRDRTVSKSLFEAYSNTEDNLTRCAISYLLKNRCKVSEKEEDLKKFAQRRRKVEIQIERLTEQLTARIPKGRDLTDTKWLETLIIATQKVPSNEAEAKSWQDNLLKKPSRVPFPVAFETNEDMTWFKNKDGRICVKFNGFSEHTFQIYCDSRQLHWFQRFYEDQQIKRIGKKQHSSSLFTLRSGLIAWQEEEGKGDPWDVNHLILYCSVDTRLWTDEGTNLVREEKAEEIAKILTKIKAKDDLNDKQLAYIKRKNSTLARINNPFPRPNKPLYKGRSHILVGVSFGLEKPTTVAVVDGSTGEVLIHRSIKQILGDNYRLLNRQRQQKHSLSHQRQIAQTLAAPNQLGESELGQYVDRLIAKEIIAISQTYKAGSIVLPKLGDMREQVQSEVQAKAEQKSDLIKVQKKYAKQYRVSVHQWSYSRLITSIQNQAKKAGIVVEEAKQSVRGSPQEKAKELAIAAYYSRKIN

SEQ ID NO: 305 - protein, 581 aa, unidentified
  source 1..581: note = MG64-57-B transposition protein
MSQDSQNSLVMDTDDEHPQIGSKGKLINSHKLPSNELLTDEVNLRMEVIQSLTEPCDRKTYAIRKKEAAEKLGVSIRQVERLLKKWREERLVGLATTRSDKGKYRLDQEWIDFIIDTFKQGNEGSKRMTRHQVFLRVKGRAKQLNLNKGEYPSHQSIYRILDEYIEQKQRKLKARSPGMLGERLTHMTRDGRELEVECSNDVWQCDHTRLDVRLVDEYGVLDRPWLTIVIDSYSRCLVGFYLGFDHPSSQIDALALRHAILPKSYSSEYQLRNEWRTYGKPNYFYTDGGKDFTSIHTTQQVAVQIGFNCALRRRPSDGGIVERFFKTLNEQVLNLLPGYTGSNVQKRPENVDKDACLTLKDLEKVIVRYIVGEYNQHTDARMKDQSRIGRWEAGLMADPYLYDELDLAICLMKRERRKVQKYGCIQFENLTYRAEHLRGRDGEIVAFRYDPVDVTTLFVYKINADGTEEFLDYGHAQGFETERLSLRELKAINKRKKEASQEINNDSILEAMLDRQAFVEQIVKQNRKQRREAANEQVNPVESVAKKFTVPEPKEIAVESEPEAELPKYEVRYMDEFYEED

SEQ ID NO: 306 - protein, 280 aa, unidentified
  source 1..280: note = MG64-57-C transposition protein
MTTNAISLAKQFGVIEEPTPEVQAEIERLSREPYLELDQVKYCHAWMYELVISRMTGLLVGDSRCGKTVTCKAFAHRYNKSRQAKGQRLKPVVYIQIPKNCGSRDFFIKILKALNKPSNGTISDLRERTLDSLAIHQVEMLIIDEANHLKFETFSDVRHIYDDDNLRISVLLVGTTSRLLAIVKRDEQVINRFLEQFELDRLEDAQFKQMIQIWERDVLRLPEESKLASGDNLKLLKQATKKLIGRLDMILRKAAIRSLLRGHKKVDKDVLKEVIAASKL

SEQ ID NO: 307 - protein, 173 aa, unidentified
  source 1..173: note = MG64-57-Q transposition protein
MEENVDEKRQLWLTRVEPYEGESISHFLGRFRRAKGNKFSAPSGLGDVAGLGAKLARWEKFYFNPFPARQELEALAAVVEVDADRLREMLPPPSVGMKHSPIRLCGDCYAESPCHKIEWQFKVTVGCYRHKLRLLSKCPVCGKPFPIPALWVEGHCPRCFTPFAQMAKSQKYY

SEQ ID NO: 308 - protein, 235 aa, unidentified
  source 1..235: note = MG64-58-B1 transposition protein
MKDAESTTNSPMTHASIVDAENGKAEANIIVSELSDEALLKMEVIQSLLKNSDRSTYGELLKQSAEKLGRSVRTIRRLVDKWEKEGLAGLVQNQRDDKGKHRVDKYWQEFVLTTYKENNKGSKRMTRQQVFIRAKARADELGIEPPSHMTVYRILKPIIDKQEQAKSIRSPGWRGSRLSVKTRDGKDLQVEHSNQVWQCDHTRVDVLLVDQHGKILSRPWLTTSIAIRVALWVLI

SEQ ID NO: 309 - protein, 355 aa, unidentified
  source 1..355: note = MG64-58-B2 transposition protein
VGINLGYDAPSSTVVALALRHAILPKQYSSEYGLHEEWGTSGLPQNFYTDGGKDFRSNHLQQIGVQLGFVCHLRDRPSEGGSVERPFKTLNTELFSTLAGYTGSNVQERPEEAEKEASFTLRQLEKMLVRYIVDNYNQRIDARMGDQTRFQRWESGLIAMPDLLSERDLDICLMKQTRRQVQRGGYLQFENLMYRGELLAGYAGESVVLRYDPKDITTILVYRIEGDKEIFLARAYAQDLETEELSLDEAKASSRKVREAGKAISNRSILAEIRERETFPTQKKTRKERQKLEQAEVKKAKQLTPAETEEEIIVVSIDAKPTAKNPLESELCTESGEPDMPEVLDYEQMREDYGW

SEQ ID NO: 310 - protein, 278 aa, unidentified
  source 1..278: note = MG64-58-C transposition protein
MVAKEAQEVAKQLGDIPVNDEKLQAEIHRLNRKGFVPLEQVQTLHDWLEGKRQSRQSGRVVGESRTGKTMGCDAYRLRNKPKQEAGKPPTVPVAYIQIPQECGAKEFFGVILEHLKYQVTKGTVAEVRDRALRVLKGCGVEMLIIDEADRFKPKTFAEVRDIFDKLEIPVILIGTDRLDAVIKRDEQVYNRFRSCHRFGKLSGEEFKRTVEIWEKKVLQLPVASNLSSKTMLKTLGEATGGYIGLMDMILRESAIRALKKGLQKIDLNTLKEVTAEYR

SEQ ID NO: 311 - protein, 167 aa, unidentified
  source 1..167: note = MG64-58-Q transposition protein
MESEYIKAWLFQVEPFEGESLSHFLGRFRRTNDLTPGGLGSQAGLGGAIARWEKFRFNPPPSLGQLEKLAVVAGIDAGRLVQMLAPPGVSMKLEPIRLCAACYAESPCHKIEWQFKETQGCKHHKLRLLSECPNCGARFRVPALWVDAWCHRCFTLFGGMVNHQKPC

SEQ ID NO: 312 - protein, 569 aa, unidentified
  source 1..569: note = MG64-59-B transposition protein
MQDAEFSTASTTKASSTDVSSTEASIIVSELSDEALLKMEVIQSLLENSDRTTYTKCLQEAAKKLGKSVRTVRRLVDKWEQEGLAGLVQNQRVDKGKHRVDENWQEFVLKTYKEGNKGSKRMTRQQVFIRAKARADELGVKPPSHMTVYRILQPLIDKIEQAKSIRSPGWRGSRLSVKTRDGKNLQVEHSNQVWQCDHTPADILLVDQHGKLLSRPWLTTVVDSYSRCIMGIHLGYDAPSSQVVALALRHAILPKQYNSEYGLHEEWGTYGLPQHLYTDGGKDFRSNHLQQIGVQLGFVCHLRDRPSEGGSVERPFKTLNTELFSTLQGYTGSNVQERPEEAEKEACLTLRQLEQMLVRYIVDNYNQRLDARMGDQTRFQRWESGLIAAPDLLSERDLDICLMKQIRRQIQRGGYLQFENLMYRGELLAGYAGESVVLRYDPRDITTILVYRTEGDKEVFIARAIAQDLETEELSLDEAKAISRRVREAGKAVSNRSILAEVREREVFPTQKKTKKERQKLEQAEVKKAKQLIPVEPEESVEVVSIDSETEPDMPEVFDYEQMREDYGW

SEQ ID NO: 313 - protein, 278 aa, unidentified
  source 1..278: note = MG64-59-C transposition protein
MSAKEAQAIAQQLGDIPVNSEKVQAEIRRLNRKGFVPLQQVQTLHDWLEGKRQSRQSGRVVGESRTGKTMGCDAYRLRNKPKQEAGKPPTVPVAYIQIPQECGAKEFFGVIMEHLKYQVTKGTVAEIRDRTLRVLKGCGVEMLIIDEADRFKAKTFAEVRDIFDKLEISVILVGTDRLDAVIKRDEQVYNRFRSCHRFGKMSGQDFKQTVEIWEKQILKLPVASNLGSKTMLKTLGEATGGYIGLMDMILREAAIRALKKGLQKIDLETLKEVAAEYR

SEQ ID NO: 314 - protein, 173 aa, unidentified
  source 1..173: note = MG64-59-Q transposition protein
MMEAENIKPWLFQVEPLDGESLSHFLGRFRRANDLTPSGLAKTAGLGGVVARWEKFRFNPPPSRQQLLSLAVVVGIDADRLTLMLPPVGMGMKMEPIRLCGACYGESPCHKIEWQFKVMQGCKLHNLSLLSECPNCGARFKVPALWVDGWCQRCFTPFGKMIEGQYKVLHSSI

SEQ ID NO: 315 - protein, 627 aa, unidentified
  source 1..627: note = MG64-61-B transposition protein
MSGFHSMADEEFEFTEGTTQVADAILLDKSNFVVDPSHIILATSDRHKLTFNLIQWLAESPNRAIKSQRKQAVANTLDVSTRQVERLLKQYDEDKLRETAGIERADKGKYRVSEYWQNFIKTIYEKSLKDKHPISPASIVREVKRHAIVDLKLKLGEYPHQATVYRILDPLIEQHKRKTRVRNPGSGSWMTVVTRDGELLKAEFSNQIIQCDHTKLDVRIVDSDRNLLSDRPWLTTIVDTYSSCIVGFRLWIKQPGSTEVALALRHAILPKKYPDDYQLNKSWDICGHPYQYFFTDGGKDFRSKHLKAIGKKLGFQCELRDRPPEGGIVERIFKTINTQVLKELPGYTGANVQERPENAEKEACLTIQDLDKILASFFCDIYNHEPYPKEPRDTRFERWFKGMGEKLPEPLDERELDICLMKEAQRVVQAHGSIQFENLIYRGEFLKAHKGEYVTLRYDPDHILSLYIYSGETDDNAGEFLGYAHAVNMDTHDLSIEELKALNKERSNARKEHFNYDALLALGKRKELVEERKEDKKAKRNSEQKRLRSASKKDSNVIELRTTRASKSLKKQENQEVLPERISREEIKFEKIEQQPQENLSTLPNPQEEERHKLVISSRKKNLQNIW

SEQ ID NO: 316 - protein, 247 aa, unidentified
  source 1..247: note = MG64-61-C transposition protein
MAQPQLATQPIVEVLAPKLDIKAQIAKTIDIEEIFRTYFITTDRASECFRWLDELRILKQCGRIIGPRNVGKSRAALHYRDDDKKRVSYVKAWSASSSKRLFSQILKDINHAAPTGKRQDLRPRLAGSLELFGLELVIIDNAENLQKEALIDLKQLFEECNVPIVLAGGKELDELLQDCDLLTNFPTLYEFERLEYDDFKKTLTTIEFDVLSLPEASNLTEGNMFEILAASTQARMGILIKISCYAA

SEQ ID NO: 317 - protein, 184 aa, unidentified
  source 1..184: note = MG64-61-Q transposition protein
MVQNIFLSKTEIEINEDDEIRPRLGYVEPYEGESISHYLGRFRRFKANSLPSGYSLGKIAGIGAVTTRWEKLYFNPFPTRQELEDLASVVGVNVDRLMEMLPSQGMTMKPRPIRLCAACYAEVPCHRMEWQFKDKIRCDRHNLRLLTKCTNCETPFPIPADWVKGECPHCSLPFAKMVKKQRRD

SEQ ID NO: 318 - protein, 620 aa, unidentified
  source 1..620: note = MG64-60-B transposition protein
MNERQDESLEAASEISDEIVLSDRAFDTDPSKILIGASDPQKLRFRLIEWLSESPNRKVKAERKQAIIKTLDVSTRQVERLLNQYYEDKLRETAGTERSDKGDHRIDDYWQDYIREVYEKSLKDKHPLKPADVVREVQRHAVIDLRHEEGDWPHPATVYRILKPLVERHKRKQKIRNPGSGSWMAVETRDGKLLKADYSNQIVQCDHTKLDIRIIDKEGKLLNWRPWLTTIVDTFSSCLIGYHLWHKQPGSHEVALTLRHAILPKQYPAEYELKKPWDIYGAPLQYFFTDGGKDLSKSKLIKAIGKKLGFQCELRDRPIQGGIVERLFKTINTEVLAALPGYISKEEGGAERAEKEACFTLEDIDKILAGYFCDDYNHKPYPKDPRDTRFERWFRGMGNKLPEPLNERDLDICLMKEEQRFVQAYGSVYFENLTYRCEELRSLKGEPVTLTYDPDHVLTLYIYRQSTGDEVREFIGYAHAINMDNQDWSLDELKQFNKVRNTAKRDHSNYDALLALNQREKLAEQRKQEKKERQRSEQKKLRGKSKQNSNVVELRKERAGKSAGSAEPIELLPERALPEMSEPQTPAPPSQTPEPAASKPPTEERHRLIIPKNQTLKRIW

SEQ ID NO: 319 - protein, 281 aa, unidentified
  source 1..281: note = MG64-60-C transposition protein
MAQSQLATQVPVEVLAPQLNLTDVLAKMVAIEELFETAFIPTDRASQCFRWLDELRLLKQCGRVIGVSEVGKSRTAKHYREDNPKRVAYVKAWTNSSSKRLFTQILKEIKHAAPNGRHKDLRSRLAGSLEPFGIELVIVDNANNLQREALLDLKQLFEESKVSIALVGGHELDTMLDDFDLLTCFPNLFEFARLDCEDFEKTLKTIELDILALPEASNLTEGKIFEILASATEARMGMLIKILTKAILRSLNKGSGKVDAGALEAIANRYGKKYKPPTAKK

SEQ ID NO: 320 - protein, 174 aa, unidentified
  source 1..174: note = MG64-60-Q transposition protein
MFEADDDQLRLGCVEAYDGESISHYLGRLRRIKANSLPSAYSLGQLVGLGGTLARWENLRFNPFPSDAELETLGKLMQVAPVKLAQMLPLKGVTHQPAPIKLCGACYAETPCHRIGWQAKSKQETLGCNRHQLRLLSKCPQCKRPFPIPSLWKEGSCNNCSLPFAKMLRFQKAL

SEQ ID NO: 321 - protein, 578 aa, unidentified
  source 1..578: note = MG64-65-B transposition protein
MGETLNSDEGNTSLAFDSSDELDEILEDEDAEPTNTIITQLSDEAKLRMEVLQSLIEPCDRKTYGIKLKQAAEKLGKTVRTVQRLVKKYQEQGLSAITEPERSDKGNYRIDEEWQEFIVKTYKEGNKSGRKMTPAQVAIRVQVRAEQLGLERYPSHMTVYRVLNPIIERKEQKQKVRNIGWRGSRVSHQTRDGQTLEPRYSNHTWQCDHTKLDVMLVDQYGETLARPWLTKITDSYSRCIMGIHLGFDAPSSLVVALAMRHAVLPKKYPSEYKLHCEWGTFGIPENLFTDGGKDFRSEHLKQIGFQLGFECHLRDRPSEGGIEERSFGTINTDFLSGFYGYLGSNIQQRPENAEEEACITLRDLQLLIVRYVVDNYNQRLDARSGQTRFQRWEAGLPALPSLIDERQLDICLMKKTRRSIYKGGYVSFENITYRGEYLAAYAGEGVILRFDPRDISTVFVYRQDSGKEVLLSQAHAIDLETEQISLEEAKAASRKIRDTGKQVSNKSILAEVRDRDVFIKQKKKSQREHKKEEQAQVHSVYKPPQIHEPVEQTQETPQLQKRRPRVYDYEQLRRDYDD

SEQ ID NO: 322 - protein, 278 aa, unidentified
  source 1..278: note = MG64-65-C transposition protein
MTDESLRRWVQNLWGDDPIPEELMPQIERLITPSVVELDHIQRIHDWLDSLRLSKQCGRIVAPPRAGKSVTCDVYKLLNKPQKRTGKRDIVPVLYMQVPGDCSAGELLTLILESLKYDATSGKLTDLRRRVLRLIKESKVEMLIIDEANFLKLNTFSEIARIYDLLKIAIVLVGTDGLDNLIKKEPYIHDRFIECYRLQLVSEKKFAELLQIWEDEVLCLPVPSNLTRKETLMPLYQKTGGKIGLVDRVLRRTAILSLRKGLSNIDKATLDEVLEWFE

SEQ ID NO: 323 - protein, 167 aa, unidentified
  source 1..167: note = MG64-65-Q transposition protein
MENIEKKTRFFDIELLQGESLSHFLGRFRRENYLTTTQLGKLTGLGAVIGRWEKFYFNPFPTQQELEALAGIVGVEVEKLREILPPKGVTMKPRPIRLCGACYAEVPYHRMKWQFKDKMKCDRHNLGLLTKCTNCETPFPIPSDWVQGECSHCFLPFATMAKRQKRY

SEQ ID NO: 324 - protein, 274 aa, unidentified
  source 1..274: note = MG64-66-B transposition protein
MQLGFECHLRDRPSEGGIEERSFGTINTDFLAGLYGYLGSNIQQRPENAEEEACITLRELHLLLVRYIVDNYNQRIDARSGNQTRFQRWEAGLPALPNLIEERQLDICLMKKTRRSIYKGGYVSFENIMYRGDYLAAYAGESVLLRYDPRDISTVFVYRQDSGKEVLLSQAHAIDLETEQISLEETKAANRKIRNTGKQLSNKSILAEVQDRDTFIKQKKKSHKERKKEEQAQVRSVKPSQTNKPVETVEEIPQPQKRRPRVFDYEQLRKDYDD

SEQ ID NO: 325 - protein, 229 aa, unidentified
  source 1..229: note = MG64-66-C1 transposition protein
MAEDYLRKWVQNFWGDDPIPEELLPIIERLITPSVVELEHIQKIHDWLDSLRLSKQCGRIVAPPRAGKSVTCDVYKLLNKPQKRTGKRDLVPVLYMQVPGDCSAGELLTLILESLKYDATSGKLTDLRRRVLRLLKESKVEMLVIDEANFLKLNTFSEIARIYDLLNVSIVLVGTDGLDNLIKKEPYIHDRFIECYRLPLVSEKKFPEFVQIWEDEVLCLPVPSNLTKS

SEQ ID NO: 326 - protein, 46 aa, unidentified
  source 1..46: note = MG64-66-C2 transposition protein
MPLYQKTSGKIGLVDRVLRRAAILSLRKGLKNIDKATLDEVLEWFE

SEQ ID NO: 327 - protein, 81 aa, unidentified
  source 1..81: note = MG64-66-Q transposition protein
MEIPAKEPRFFQVEPLEGESLSHFLGRFRRENYLTATQLGKLTGIGAVISRWEKFYLNPFPTPQELEALAAVQNEFGNSRH

SEQ ID NO: 328 - protein, 579 aa, unidentified
  source 1..579: note = MG64-68-B transposition protein
MGETLNSNEVDESLVLYDGSDEVDEISESEDTKQSNVIVTELSEEAKLRMEVLQSLIEPCDRKTYGIKLKQAAEKLGKTVRTVQRLVKKYQEQGLSGVSEVERSDKGGYRIDDDWQDFIVKTYKEGNKGGRKMTPAQVAIRVQVRAGQLGLEKYPCHMTVYRVLNPIIERKEQKQKVRNIGWRGSRVSHQTRDGQTLDVHHSNHVWQCDHTKLDVMLVDQYGETLARPWLTKITDSYSRCIMGIHLGFDAPSSLVVALAMRHAMLRKQYSSEYKLHCEWGTYGVPENLFTDGGKDFRSEHLKQIGFQLGFECHLRDRPPEGGIEERGFGTINTDFLSGFYGYLGSNVQERPEGAEEEACITLRELHLLILRYIVDNYNQRIDARSGNQTRFQRWEAGLPALPNLVNERELDICLMKKTRRSIYKGGYVSFENIMYRGDYLSAYAGESVLLRYDPRDISTVFVYRQDSGKEVLLSQAHAIDLETEQISLEETKAASRKIRNAGKQLSNKSILAEVQDRDTFIKQKKKSHKQRKKEEQAQVHSVKPPQTNEPVETVEEIPQPQKRRPRVFDYEQLRKDYDD

SEQ ID NO: 329 - protein, 278 aa, unidentified
  source 1..278: note = MG64-68-C transposition protein
MAEDYLRKWVQNLWGDDPIPEELLPIIERLITPSVVELEHIQKIHDWLDSLRLSKQCGRIVAPPRAGKSVTCDVYKLLNKPQKRTGKRDIVPVLYMQVPGDCSAGELLTLILESLKYDAISGKLTDLRRRVLRLLKESKVEMLVIDEANFLKLNTFSEIARIYDLLKISIVLVGTDGLDNLIKKEPYIHDRFIECYRLPLVSEKKFPEFVQIWEDEVLCLPVPSNLTKSETLMPLYQKTSGKIGLVDRVLRRAAILSLRKGLKNIDKATLDEVLEWFE

SEQ ID NO: 330 - protein, 167 aa, unidentified
  source 1..167: note = MG64-68-Q transposition protein
MEIPAKEPRFFQVEPLEGESLSHFLGRFRRANYLTATQLGKLTGIGAVISRWEKFYLNPFPTPHELEALAAVVEVKVDRLSEMLPPKGVTMKPRPIRLCGACYQESPCHRVEWQFKDVMVCDRHNLRLLTKCTNCETPFPIPADWVQGECPHCFLPFATMAKRQKAC

SEQ ID NO: 331 - protein, 545 aa, unidentified
  source 1..545: note = MG64-67-B transposition protein
MGKRLNSNEVDESLLLHDDSDEVDEISESEDAKENNVIVTELSEEAKLRMQVLQNLVEPCDRKTYGIKLKQAAEKLGKTVRTVQRLVKKYQQQGLSGIIEVERSDKGDYRIDDDWQDFIVKTYKEGNKGGRKMTPAQAAIRVQVRAGQLGLDKYPCHMTVYRVLNPIIERKEQKQKVRNIGWRGSRVSHQTRDGQTLDVHHSNHVWQCDHTKLDVMLVDQYGETLARPWLTKITDSYSRCIMGIHLGFDAPSSLVVALAMRHAMLRKQYSSEYKLHCEWGTYGVPENLFTDGGQDFRSEHLKQIGFQLGFECHLRDRPPEGGIEERGFGTINTDFLSGFYGYLGSNVQARPEGAEEEACITLRELHLLIVRYIVDNYNQRIDARSGNQTRFQRWEAGLPALPNLVNERELDICLMKKTRRNIYKGGYVSFENIMYRGDYLSAYAGESVLLRYDPRDISTVFVYRQDSGKEVLLSQAHAIDLETEQISWEEAKAASRKIRNAGKQLSNKSILAEVQDRDTFIKQKKKSHNISDTAGVRSVGCCGGS

SEQ ID NO: 332 - protein, 99 aa, unidentified
  source 1..99: note = MG64-67-Q transposition protein
MAAVVEVKVDRLIEMLPPKSVTMKPRPIRLCGACYQESPCHRVEWQFKDVMVCDRHQLRLLTKCTNCETPFPIPADWVQGECPHCLLPFATMARRQKRC

SEQ ID NO: 333 - protein, 254 aa, unidentified
  source 1..254: note = MG64-71-C transposition protein
MSQDSQNSLVMDTDDEHPQVRYKGKLINSHKLPSNELLTDEVNFRMEVIQSLTEPCDRKTYAIRKKEAAEKLGVSIRQVERLLKKWREERLVGLATTRSDKGKYRLDQEWIDFIIDTFKKGNEGSKRMTRHQVFLRVKGRAKQLNLNKGEYPSHQSVYRILDEYIEEKQRKLKARSPGMLGERLTHMTRDGRELEVECSNDVWQCDHTRLDVRLVDEYGVLDRPWLTIVIDSYSRCLVGFYLGFDNPSSQIDAL

SEQ ID NO: 334 - protein, 165 aa, unidentified
  source 1..165: note = MG64-71-Q transposition protein
MPYEGESISHFLGRFRREKGNKFSAASGLGDVAGLGAVLARWEKFYFNPFPTHQELEALATVVEVEADRLREMLPPPGVGMKHSPIRLCGHCYAESPCHQIEWQFKVTVGCVSEAPLKEARHKLRLLSKCPICEKPFTIPALWVEGHCPRCFTSFAEMAKYQKHY

SEQ ID NO: 335 - protein, 742 aa, unidentified
  source 1..742: note = MG64-72-B transposition protein
MNLLEASEFSDQQDDRDFSLIYEEEDTSIEEPPIADEIEGLGTGKPETTYQPSEQSIACYGVPEDDTAVEFLDRRYALEDEIILENGEKVRLQLTEEQKLKREVIRSLLEVRHNRKLFSEQLKEGAKKLNLSTRQVRRIFQDWVDLGITSLQKAPRSDRGKPRRNKYWYDLSVKIYKDGNKGNRSMTRTQVADRIQDQIYEYVKPELPSEVSKLQEAGFSGEALDQELSRLIEERQETCQQQQQEKQEEIAELKTKVSELRKRKLDTQAAHSRIKQLKEQKQAIVAFEFWRTYGQPPCTRTVEIWLKPIEEKNHKANTSRNPGWHGDTLILRTRKGEAISVTRSNQVWQIDHTKADVLLVDEDGVEIGRPILTTVIDCHSRCIVGYRLGLKAPSSLVVALALRHAMLPKQYGPEYEMRCQWITHGSPRYIYTDGAADFNFLEHVGDQLGFKAERREQPSQGGIVERPFRTLSELLSEAPGYTGPNVQKRPKDVAKVVKMTLRELDMMLAGYFADNYNQAADPRTRANPFVPEQSRMARWEAGLKTPPAVIKPRELDICLMQTDERVVYDNGYVRFANLLYKGENLGKHAKDPVFLRFDPRDITLLLVYSRQDGREKFIARAYAVGLEADQLSLEEVKHSAKKLEKAGKQINNIAIREETIRRRKLFASKQNITRTDRRNAEEAKANPVPERYEEDQHKRVLKPPVEEQPRPVDVPIDVSIALPEPFIDDDDDSEDYAIEVDD

SEQ ID NO: 336 - protein, 381 aa, unidentified
  source 1..381: note = MG64-72-C transposition protein
MQNQLSEGIATLSQAKESVPQEKLLPALDILSQPKALTQDLLLSLIAALSNPQEQVDDLIAIAKEMFAAMACIAEPEPNILRLDQVSSFIDWIQGRLKLRKPGKAIGETGLGKTCACHACLEEFEPVQHPNQPSERPFVYVQIDENRCAPGRFLQLILIALRKPTSGNLHQLKQRVKKFFKQYKVQILLVDEAHCLHFDALKTARSFYDDKDLGVIPILIGTSNRLDTLMEKDEQVNNRFANTFVFEELAGDKFSGVVKIWGEKIIRMQDPLISKDPSKAGGKKRIISLLKTGKAGINKDLASTLEEMTSGELRLLDNILRDAAVRLLEKKLGEIYSLVLQALKTDSKVLAFDAKKILKEIRIDKSFLQSMKGEYLRGRSM

SEQ ID NO: 337 - protein, 173 aa, unidentified
  source 1..173: note = MG64-72-Q transposition protein
MQFEPHRIELERVEPFPGESISHFLGRFERANVWTTYQIGRVTGIKAGVSRWKKLYLSPLFPTKQELEVLAELVEVSADRLAEMLPSESQAMKPPGTIYLCAACYTEMPCHLIQWQFKGVNACDRHRLYLLSKCPKCKKTFSAPADWEEGICFHCGKSFFEMVVHQGRVKNAK

SEQ ID NO: 338 - protein, 649 aa, unidentified
  source 1..649: note = MG64-85-B transposition protein
MEASQVACLDTDLQAMDEVMLSDRAFDTDPLKILIESAEPQKLRFQLIQWLAESPNRTVKAERKEVIVQTLAISTRQVERMLNQYQEDRLRETTGIERSDKGQHRIHNYWQNYIREVYEKSLKDKHPLKPADVVREVQRHAVIDLRHEEGDYPHPATIYRILKPLVERQKRRQKIRNPGSGSWLAVETRDGKLLRADYSNQIIQCDHTKLDIRIADKDRKLLNWRPWLTTVVDTFSSGLIGYHLWHKQPGAHEVALTLRQAILPKRFPPDYAISKPWCYGPPLQYFFTDGGKDLSKSKLIKAIGKKLGFRCELRDRPTQGGIVERLFQTINTEFLQCLPGYISKEEGGAERAEKEACLTFEDIDKFLAGYFCEYNHDLYPKDESETRFERWFRGMGKQLPEPLDERALDICLMKEEQRVVQAHGSIYFENLTYRCEELRSLKGEYVTLSYDPDHVLTLYIYRQATSDDAVEFIGYAHAINMDTQDLSLDELKQLNKVRSNARREHSNYDALLALDTRQKLVEQRKQEKKERQRSEQRKLREKSKQNSNVVDMRKARAGKSTSRAEPMELLPERISSEQLKLQSPVPIPGISEPAGLLTEERHNLVSASSQTSKIAVPPPQISETAASAPSSEERHRLIIPKNQTLKRIW

SEQ ID NO: 339 - protein, 288 aa, unidentified
  source 1..288: note = MG64-85-C transposition protein
MAQSELAVQVPVEVLAPQLDITDVIAKTAAIEELFKTAFIPTDRASQYFRWLDELRLLKQCGRVIGPKGVGKSRSSKHYREEDRKRVSHIAAWSNSSSKKLFSQILKDINHAAPRVRRHDLRDRLAGCLEPFGIELLLVDNAENLQREALVDLKQLHEESGVPVILIGEQDLDNNLENADLLTCFPTLFEFDKLDYEDLRKTLRTIELDLLALPQASNLAEGNLLEILAVSTQAHMGTLIKLLTKVVLHSLNKGHMKVDEAILRNIASRYGKRYISPEARKKPELGEG

SEQ ID NO: 340 - protein, 176 aa, unidentified
  source 1..176: note = MG64-85-Q transposition protein
VSQPEANEPRLGSVEPLEHESISHYLGRLRRLKANSLPSAYALGQAAGIGGITVRWEKLYFNPFPTEAGLGAIARLIGLDTRRLQAMLPSQGMTLQPRPIRLCGACYAEMPCHQMSWQHKDIVAVCPHHPLRLLERCPSCKKPFQIPALWMDGKCHHCGIWFTAMVKYQERIKKTG

SEQ ID NO: 341 - protein, 128 aa, unidentified
  source 1..128: note = MG64-86-Q transposition protein
MAGLGGAIARWEKFYHNPFPTLQQLEALATVIGVEADKLANMLPPAGVGIKHEPTRLCGACYAEFACHRIEWQFKTTNRCERHQLRLLSECPNCKARFKVPALWVDGHCQRCFMKFAEMAKYQKPVLQ

SEQ ID NO: 342 - protein, 304 aa, unidentified
  source 1..304: note = MG64-49-C transposition protein
MSQTHLASVPDPSINNRSATQQSPQLWQKPIRSPEIQAEVERIGTIDPYVAVGRDEALFTCLNNWRDRRTCGRIMTVDRLGLFKDLDYYTNQQTRRRGDLIRMPAPVAYIEIDDPGNGKNVFLSILDFLANPVSCGNPRDLRLRTWATIKKCGVKILVVNYADLLLFSGLNELMRISEKCGISVILCGTSRLDEILDAKHRKRYLPIHNTFLNHHKLSVLSASEIATVIEQWENTLGCSKSMGLATDERIVKILNQLSDGQIQPLYDLLKRISIWHLDNPHVEINVNNVSTLLTTVQAPQVGLE

SEQ ID NO: 343 - protein, 177 aa, unidentified
  source 1..177: note = MG64-49-Q transposition protein
MTEPTAECFSIALQPYEDESMSHYLGRWKRQDVVSLSSIGSLSRQLRLGTAMSRWEKFYLNPFPTLKQLEQLGKVMGIEGERLLLMFPPKGEPINVRIIRLCCACYDEAHYHRMRWQFQSTAACDRHQLRLLYKCPNCEQHLPIPAEWESGRCKKCQMLFRSMIKHQKPARTEGEKS

SEQ ID NO: 344 - protein, 173 aa, unidentified
  source 1..173: note = MG108-1-Q transposition protein
VETQTEQALPWQIQPYEGESISHYLGRVRRADAISASSASGLSKALGLGIALARWEKLRFNPYPSRVELEALDKVVGLGVDRLADLFPPKDEPIRLQPIRFCPACYVEFPYHRMKWQYQSTAGCDRHHLKLLCKCPGCKDYFQVPSLWTEPKCKRCGMPFKRMVKHQKPHEGD

SEQ ID NO: 345 - protein, 621 aa, synthetic construct
MKKLFAQDVNIDTEVISNQIPTSDPSQSNLIASELPEEARPKLEVIQSLLEPCDRVTYGERLREGAEKLGLSVRSVQRLFKKYQEKGLIALLSGSRTDKGEHRISELWQNFIVKTYQEGNKGSKRMSPKQVALKVQAKAGAIADDNPPSYRTVLRVLKPILEKQEKAKSIRSPGWRGSTLSVKTRDGDDLDISYSNQVWQCDHTRADVLLVDQHGKLLVRPWLTTVIDSYSRCIMGINLGFDAPSSQVVALALRHAILPKRYGTEYKLNCDWGTYGTPEYLFTDGGKDFRSNHLAEIGLQLGFVCKLRDRPSEGGIVERPFKTLNQSLFSTLPGYTGSNVQERPEDAEKDAQLTLRDLEQLIVRFIVDRYNQSIDARMGDQTRYQRWEAGLQKEPDVISERDLDICLMKMSRRTVQRGGHLQFENVMYLGEYLAGYAGEVVSFRYDPRDITTIWVYRQENDREVFLTRAHAQGLETEQLSVDDAKASAKRLRAAGKTISNQSILQETIEREVLAERTKSRKHRQKEEQSYKRSPSAAVMVEVESEQLEIESSNEANANSVSADIEVWDYDEMREGLGWGGGGSGGGGSYPYDVPDYAGSGSPKKKRKVDGSPKKKRKVDSG

SEQ ID NO: 346 - protein, 618 aa, synthetic construct
MKRPAATKKAGQAKKKKYPYDVPDYAGGGGSGGGGSGGGGSKKLFAQDVNIDTEVISNQIPTSDPSQSNLIASELPEEARPKLEVIQSLLEPCDRVTYGERLREGAEKLGLSVRSVQRLFKKYQEKGLIALLSGSRTDKGEHRISELWQNFIVKTYQEGNKGSKRMSPKQVALKVQAKAGAIADDNPPSYRTVLRVLKPILEKQEKAKSIRSPGWRGSTLSVKTRDGDDLDISYSNQVWQCDHTRADVLLVDQHGKLLVRPWLTTVIDSYSRCIMGINLGFDAPSSQVVALALRHAILPKRYGTEYKLNCDWGTYGTPEYLFTDGGKDFRSNHLAEIGLQLGFVCKLRDRPSEGGIVERPFKTLNQSLFSTLPGYTGSNVQERPEDAEKDAQLTLRDLEQLIVRFIVDRYNQSIDARMGDQTRYQRWEAGLQKEPDVISERDLDICLMKMSRRTVQRGGHLQFENVMYLGEYLAGYAGEVVSFRYDPRDITTIWVYRQENDREVFLTRAHAQGLETEQLSVDDAKASAKRLRAAGKTISNQSILQETIEREVLAERTKSRKHRQKEEQSYKRSPSAAVMVEVESEQLEIESSNEANANSVSADIEVWDYDEMREGLGW

SEQ ID NO: 347 - protein, 326 aa, synthetic construct
MKRPAATKKAGQAKKKKGGGGSGGGGSGGGGSDYKDDDDKNATIKENSSQEKPASQIAEELGDFKVDSQLLQIEIARLNKKSIVPLEHIKDLHDWLDEKRKARQSCRLVGESRTGKTVACEAYTFRNKPKQEGKQAPTVPVVYIMPPAKCGAKELFREIIEYLKYRAVRGTVADFRSRAMEVLKGCEVEMIIIDEADRLKPETFSDVRDINDKLGIAVVLVGTDRLDAVIKRDEQVYNRFRASRRFGKLTGEDFKRTVEIWEDKVLKMPVASNLTNKEMLKILLKATEGYIGRLDEILREAAIKSLSRGFRKVEKAVLQEVAREYS

SEQ ID NO: 348 - RNA, 238 nt, synthetic construct
  source 1..238: mol_type = other RNA
gtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccgaaaggatgggttgaaagnnnnnnnnnnnnnnnnnnnnnnn

SEQ ID NO: 349 - skipped

SEQ ID NO: 350 - RNA, 241 nt, synthetic construct
  source 1..241: mol_type = other RNA
ataacagcgccgcaggtcatgccgtcaaaagcctgaaagggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccagaaatgggttgaaagnnnnnnnnnnnnnnnnnnnnnnn

SEQ ID NO: 351 - RNA, 230 nt, synthetic construct
  source 1..230: mol_type = other RNA
ataacagcgccgcaggtcatgccgtcaaaagcctctgaactgtgttaaatgggggttagtttgactgttgaaagacagttgtgctttctgaccctggtagctgcccaccctgatgctgaaagtaataggggtgtagatgtactacagtggtggctactaaatcacctccgaccaaggaggaatccatccgaaaggatgggttgaaagnnnnnnnnnnnnnnnnnnnnnnn

SEQ ID NO: 352 - RNA, 222 nt, synthetic construct
  source 1..222: mol_type = other RNA
ataacagcgccgcaggtcatgccgtcaaaagcctgaaagggttagtttgactgttgaaagacagttgtgctttctgaccctggtagctgcccaccctgatgctgtaaggtgcgctcccagtaataggggtgtagatgtactacagtggtggctactaaatcacctccgaccaaggaggaatccagaaatgggttgaaagnnnnnnnnnnnnnnnnnnnnnnn

SEQ ID NO: 353 - RNA, 240 nt, synthetic construct
  source 1..240: mol_type = other RNA
ataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggagaaagnnnnnnnnnnnnnnnnnnnnnnn

SEQ ID NO: 354 - DNA, 119 bp, synthetic construct
  source 1..119: mol_type = other DNA
tgtacagtgactaattatttgacgtgatgccaaattgttgtcgctgatgaaaactattgatttctctatattctagctgttttccttgattaaagatcgctagccgttagtgacaaatt

SEQ ID NO: 355 - DNA, 133 bp, synthetic construct
  source 1..133: mol_type = other DNA
tgtacagtgactaattatttggtcctcccaatcagtgacaatttagctgtcgtcgttctcaaagaagagaatttgaggagtgacagattgattgtcgctttcttttttgtaagctaggatagcattatgtgtt

SEQ ID NO: 356 - DNA, 146 bp, synthetic construct
  source 1..146: mol_type = other DNA
tgtacagtgactaattatttgacgtgatgccaaattgttgtcgctgatgaaaactattgatttctctatattctagctgttttccttgattaaagatcgctagccgttagtgacaaattaagtgtcgtcctcccaatcagtgacaa

SEQ ID NO: 357 - DNA, 158 bp, synthetic construct
  source 1..158: mol_type = other DNA
tgtacagtgactaattatttgacgtgatgccaaattgttgtcgctgatgaaaactattgatttctctatattctagctgttttccttgattaaagatcgctagccgttagtgacaaattaagtgtcgtcctcccaatcagtgacaatttagctgtcgt

SEQ ID NO: 358 - DNA, 161 bp, synthetic construct
  source 1..161: mol_type = other DNA
tgtacagtgactaattatttggctagccgttagtgacaaattaagtgtcgtcctcccaatcagtgacaatttagctgtcgtcgttctcaaagaagagaatttgaggagtgacagattgattgtcgctttcttttttgtaagctaggatagcattatgtgtt

SEQ ID NO: 359 - DNA, 104 bp, synthetic construct
  source 1..104: mol_type = other DNA
tgtacagtgacacattaattgtcatcaatgacagattgctgtcgtggagccaaattatgtgtcgctgagacaaattaatgtcgtttaactatcagtgacaaatt

SEQ ID NO: 360 - DNA, 124 bp, synthetic construct
  source 1..124: mol_type = other DNA
tgtacagtgacacattaattgtcatcaatgacagattgctgtcgtggagccaaattatgtgtcgctgagacaaattaatgtcgtttaactatcagtgacaaatttttgtcgcttttcacaacaa

SEQ ID NO: 361 - DNA, 124 bp, synthetic construct; identical to SEQ ID NO: 360
  source 1..124: mol_type = other DNA

SEQ ID NO: 362 - DNA, 165 bp, synthetic construct
  source 1..165: mol_type = other DNA
tgtacagtgacacattaattgtcatcaatgacagattgctgtcgtggagccaaattatgtgtcgctgagacaaattaatgtcgtttaactatcagtgacaaatttttgtcgcttttcacaacaatagtgtgaaggaagtgcgcctttcaatccatcctagaaatt

SEQ ID NO: 363 - DNA, 186 bp, synthetic construct
  source 1..186: mol_type = other DNA
tgtacagtgacacattaatttcgtttaactatcagtgacaaatttttgtcgcttttcacaacaatagtgtgaaggaagtgcgcctttcaatccatcctagaaattataattccaatccctacttacctagaatggtggttgaaactgtaagattcgcgccgctaataaaactttcagcgatttgga

SEQ ID NO: 364 - RNA, 37 nt, synthetic construct
  source 1..37: mol_type = other RNA
gttgcaggaagcgatctggcgcgagataggatgaaag

SEQ ID NO: 365 - RNA, 37 nt, synthetic construct; identical to SEQ ID NO: 242
  source 1..37: mol_type = other RNA

SEQ ID NO: 366 - RNA, 37 nt, synthetic construct; identical to SEQ ID NO: 242
  source 1..37: mol_type = other RNA

SEQ ID NO: 367 - RNA, 36 nt, synthetic construct
  source 1..36: mol_type = other RNA
gggggaatcctatgcaagccatagttgcattgaaag

SEQ ID NO: 368 - RNA, 36 nt, synthetic construct
  source 1..36: mol_type = other RNA
gtacccaaagccttttttccttaagcctatccgcac

SEQ ID NO: 369 - RNA, 37 nt, synthetic construct
  source 1..37: mol_type = other RNA
atcgcgatcgccgtcccagctttgggcgggttgaaag

SEQ ID NO: 370 - RNA, 37 nt, synthetic construct
  source 1..37: mol_type = other RNA
gtttcaaccgccatcccagctaggggtgggttgaaag

SEQ ID NO: 371 - RNA, 38 nt, synthetic construct
  source 1..38: mol_type = other RNA
ggttgcatctgcttttcagcaactagggcgggggaaag

SEQ ID NO: 372 - RNA, 39 nt, synthetic construct
  source 1..39: mol_type = other RNA
gttgcatccgctttccagcaaccagggcgggtgaaagtt

SEQ ID NO: 373 - RNA, 37 nt, synthetic construct; identical to SEQ ID NO: 249
  source 1..37: mol_type = other RNA

SEQ ID NO: 374 - RNA, 37 nt, synthetic construct; identical to SEQ ID NO: 249
  source 1..37: mol_type = other RNA

SEQ ID NO: 375 - RNA, 37 nt, synthetic construct
  source 1..37: mol_type = other RNA
taaacaaatctactactcaacatagctttgccaaacg

SEQ ID NO: 376 - RNA, 37 nt, synthetic construct
  source 1..37: mol_type = other RNA
gtaacaataaccctccccgtgtagggcgggttgaaag

SEQ ID NO: 377 - RNA, 37 nt, synthetic construct
  source 1..37: mol_type = other RNA
gtttcaatgccccttcaagctttgggcgggttgaaag

SEQ ID NO: 378 - RNA, 37 nt, synthetic construct
  source 1..37: mol_type = other RNA
gtttcaacgccgcttccagcttgaggcgggttgaaag

SEQ ID NO: 379 - RNA, 37 nt, synthetic construct
  source 1..37: mol_type = other RNA
gtcgcaatctgcctctcagagatgggtgggctgaaag

SEQ ID NO: 380 - RNA, 37 nt, synthetic construct
  source 1..37: mol_type = other RNA
gtttcaactaccatcccgactaggggtgggttgaaag

SEQ ID NO: 381 - RNA, 39 nt, synthetic construct
  source 1..39: mol_type = other RNA
gttgcatcagccctcccagcgttgggcgggttgaaagaa

SEQ ID NO: 382 - RNA, 37 nt, synthetic construct
  source 1..37: mol_type = other RNA
gttacaatcgcctgtccgaattggggcaggttgaaag

SEQ ID NO: 383 - RNA, 38 nt, synthetic construct
  source 1..38: mol_type = other RNA
ggtttcaatcgctcccatcgatgggagcaggttgaaag

SEQ ID NO: 384 - RNA, 37 nt, synthetic construct
  source 1..37: mol_type = other RNA
gtttcaacaaccattccggctaggggtgggttgaaag

SEQ ID NO: 385 - RNA, 37 nt, synthetic construct; identical to SEQ ID NO: 384
  source 1..37: mol_type = other RNA

SEQ ID NO: 386 - RNA, 37 nt, synthetic construct
  source 1..37: mol_type = other RNA
gtttcaacaaccatcccggctaggggtgggttgaaag

SEQ ID NO: 387 - RNA, 38 nt, synthetic construct
  source 1..38: mol_type = other RNA
ctttcaacccacccctagccgggatggttgttgaaact

SEQ ID NO: 388 - RNA, 37 nt, synthetic construct
  source 1..37: mol_type = other RNA
gtttctactgccctcctgacctacggtgggctgaaag

SEQ ID NO: 389 - RNA, 37 nt, synthetic construct
  source 1..37: mol_type = other RNA
gtttcatcaaccttcccgctcttgggtgggttgaaag

SEQ ID NO: 390 - RNA, 37 nt, synthetic construct
  source 1..37: mol_type = other RNA
gtaacaataaccctcctggtgtagggtgggttgaaag

SEQ ID NO: 391 - RNA, 38 nt, synthetic construct
  source 1..38: mol_type = other RNA
ggcgcaatagccttcctagacacggataagctgaaaga

SEQ ID NO: 392 - RNA, 39 nt, synthetic construct
  source 1..39: mol_type = other RNA
gtttcaacaaccatcccgatacgagggtgggttgaaaga

SEQ ID NO: 393 - RNA, 37 nt, synthetic construct
  source 1..37: mol_type = other RNA
gtcgcaatggccgttttggccggagaagggatgaaag

SEQ ID NO: 394 - RNA, 37 nt, synthetic construct
  source 1..37: mol_type = other RNA
gacgcaatcgccttcccagagatgggtgggctgaaag

SEQ ID NO: 395 - RNA, 37 nt, synthetic construct
  source 1..37: mol_type = other RNA
gtttcatcaaccctcccgcaacagggtgggttgaaag

SEQ ID NO: 396 - RNA, 37 nt, synthetic construct
  source 1..37: mol_type = other RNA
gtttcatcagccctcccgcctatgggtgggttgaaag

SEQ ID NO: 397 - RNA, 37 nt, synthetic construct
  source 1..37: mol_type = other RNA
gtttcatcagccctcccgcctctgggtgggttgaaag

SEQ ID NO: 398 - RNA, 37 nt, synthetic construct
  source 1..37: mol_type = other RNA
gtttcaacgaccatcccagctaggggtgggttgaaag

SEQ ID NO: 399 - RNA, 37 nt, synthetic construct
  source 1..37: mol_type = other RNA
gttgcaacagccctctcagagatgcgtgggttgaaag

SEQ ID NO: 400 - RNA, 38 nt, synthetic construct
  source 1..38: mol_type = other RNA
gtttcatcagccctcccgccttggggtgggttgaaaga

SEQ ID NO: 401 - RNA, 37 nt, synthetic construct
  source 1..37: mol_type = other RNA
ggcgcaacagcccttttaggcatgggtgagttgaaag

SEQ ID NO: 402 - RNA, 37 nt, synthetic construct; identical to SEQ ID NO: 255
  source 1..37: mol_type = other RNA

SEQ ID NO: 403 - RNA, 37 nt, synthetic construct
  source 1..37: mol_type = other RNA
gtcgccatcgacttcctggcaacaggtgggttgaaag

SEQ ID NO: 404 - RNA, 35 nt, synthetic construct
  source 1..35: mol_type = other RNA
cgccatcgccctcccagagatgggcgggctgaaag

SEQ ID NO: 405 - RNA, 37 nt, synthetic construct
  source 1..37: mol_type = other RNA
gtttcaacgaccatcccgactaggggtgggttgaaag

SEQ ID NO: 406 - RNA, 37 nt, synthetic construct
  source 1..37: mol_type = other RNA
gtttcatcgaccctcccgcctttgggtgggttgaaag

SEQ ID NO: 407 - RNA, 39 nt, synthetic construct
  source 1..39: mol_type = other RNA
gttgcgatcgccttttcagctcgatgagggttgaaagat

SEQ ID NO: 408 - RNA, 37 nt, synthetic construct; identical to SEQ ID NO: 242
  source 1..37: mol_type = other RNA

SEQ ID NO: 409 - RNA, 38 nt, synthetic construct
  source 1..38: mol_type = other RNA
gtttcaatggccatctcgattaggggtgggttgaaaga

SEQ ID NO: 410 - RNA, 37 nt, synthetic construct
  source 1..37: mol_type = other RNA
gtttcaatgaccctcccgaatgggggtaggttgaaag

SEQ ID NO: 411 - RNA, 37 nt, synthetic construct
  source 1..37: mol_type = other RNA
gtcgcaactgccccttcattcgaagggaggttgaaag

SEQ ID NO: 412 - RNA, 38 nt, synthetic construct
  source 1..38: mol_type = other RNA
ggtcgccatcagcgattcagccggtggtggatagaaag

SEQ ID NO: 413 - RNA, 37 nt, synthetic construct
  source 1..37: mol_type = other RNA
gtcgcaacgggcttttaccgtctgtgaggattgaaac

SEQ ID NO: 414 - RNA, 37 nt, synthetic construct; identical to SEQ ID NO: 249
  source 1..37: mol_type = other RNA

SEQ ID NO: 415 - RNA, 37 nt, synthetic construct
  source 1..37: mol_type = other RNA
gtcgcaatcctctgcgcgggtgtggggatgttgaaag

SEQ ID NO: 416 - RNA, 37 nt, synthetic construct
  source 1..37: mol_type = other RNA
gtttcaacacccctcccagctagaggcgggttgaaag

SEQ ID NO: 417 - RNA, 222 nt, synthetic construct; identical to SEQ ID NO: 262
  source 1..222: mol_type = other RNA

SEQ ID NO: 418 - RNA, 230 nt, synthetic construct
  source 1..230: mol_type = other RNA
ataacagcgccgcaggtcatgccgtcaaaagcctctgaactgtgttaaatgggggttagtttgactgttgaaagacagttgtgctttctgaccctggtagctgcccaccctgatgctgctatctttcgggataggaataaggtgcgctcccagtaataggggtgtagatgtactacagtggtggctactaaatcacctccgaccaaggaggaatccatccttaatttttt

SEQ ID NO: 419 - RNA, 244 nt, synthetic construct
  source 1..244: mol_type = other RNA
ctaaaaatcgtgccgtagactatgtgcaaacctctggtctgcgaaaaataagacttagtttgggaagcttgatgtttcccttgctttctgggtctagcgactaaccactccgaagctgctgctagtaagcggtgttcccactggacacaagtgaatctggcagaataggggcacacccagcaagagaggacagactactgtagtgttggttgctgcttcacccccgatcaagggggaatcctat

SEQ ID NO: 420 - RNA, 222 nt, synthetic construct; identical to SEQ ID NO: 168
  source 1..222: mol_type = other RNA

SEQ ID NO: 421 - RNA, 222 nt, synthetic construct; identical to SEQ ID NO: 168
  source 1..222: mol_type = other RNA

SEQ ID NO: 422 - RNA, 226 nt, synthetic construct; identical to SEQ ID NO: 172
  source 1..226: mol_type = other RNA

SEQ ID NO: 423 - RNA, 226 nt, synthetic construct; identical to SEQ ID NO: 172
  source 1..226: mol_type = other RNA

SEQ ID NO: 424 - RNA, 203 nt, synthetic construct; identical to SEQ ID NO: 176
  source 1..203: mol_type = other RNA

SEQ ID NO: 425 - RNA, 203 nt, synthetic construct; identical to SEQ ID NO: 176
  source 1..203: mol_type = other RNA

SEQ ID NO: 426 - RNA, 277 nt, synthetic construct
  source 1..277: mol_type = other RNA
gattgcgcctcgatcgatgctctatgagccgctcgatcgtagaaaaatgggtgagtttgattatctacttcgttagataatgctgctttccgaccctggcattctgtccgcccttgaagctgcttctcatggactagcgtaagctcgttggtaagaaggaaaagtcataatttaaagtcacgtctttctagtatgacataggtgcgctcccacgcaatatagggttcagcttttattttataaaagtagagactttcctctagtgacagtgccgaaa

SEQ ID NO: 427 - RNA, 72 nt, synthetic construct
  source 1..72: mol_type = other RNA
ctattaatcgcgccgcgttgaatgttagcaataaccgctccaacgtgttaaatgagggtttgtttgacagta

SEQ ID NO: 428 - RNA, 72 nt, synthetic construct; identical to SEQ ID NO: 427
  source 1..72: mol_type = other RNA

SEQ ID NO: 429 - RNA, 220 nt, synthetic construct
  source 1..220: mol_type = other RNA
taaataatagcgccgcagttcatgttcttaggaaccgctgaactgtgaaaaatctgggttagtttgactattggaagatagtcttgctttctgaccctagtagctgctcaccccgatgctgctgtctgcggacaggaattaggtgcgctcccagcaaaaagggcgcggatatactgctgtagtggctaccaaatcaccctcgaccaagggggaacccatc

SEQ ID NO: 430 - RNA, 225 nt, synthetic construct
  source 1..225: mol_type = other RNA
taacaaacagcgccgcagttcatgcgtcttatggcgcctctgtgctgtgcaaaatgtgggttagtttgactgttggaagacagtcttgctttctgaccctggtagctgcccaccttgaagctgctatcccttgtggataggaatcaggtgcgcccccagtaatagaggtgcgggtttaccgcagtggtggctaccgaatcacctccgagcaaggaggaacccacc

SEQ ID NO: 431 - RNA, 279 nt, synthetic construct
  source 1..279: mol_type = other RNA
taatcaacagcgccgttgttcatgcgtttttacgcctctgagcaatgataaatttgggttagtttgactgttagaaatacagttttgctttctgaccctggtagctgcccaccttgaagctgctatctcttgtagataggaaataaggtgcgcccccagtaatagaggtgcgggtttaccgcagtggtgagtccacttaccgggaaaacgttcttttcggtaaagtggcgaatccgaagggtggctatcgaatcacctccgatcaaggaggaacccact

SEQ ID NO: 432 - RNA, 305 nt, synthetic construct
  source 1..305: mol_type = other RNA
tgactaacagcgccgcagttcatgctccttgagccgctgaactgtgaaaaatgaggggtcagtttggtcgttgtgagacgatcgtgctttccgcccctaagtagctgcccggtcactgactgccatcttggtcgttgttcgattatgaacaactgatggggatgggaagttgcgtggatgagttgctcttccaatgtaacgcaggtgcgcgcccagcagaagtgatcccagccttcagcaatgaaggtacagcctggttctgtagtggcagctactgattgtctccgagcaaggaggagtcctcc

SEQ ID NO: 433 - RNA, 228 nt, synthetic construct
  source 1..228: mol_type = other RNA
tcagcaatagcgcgccgttaccaaacggtgttaagtaaaggggtcagtttaattgctttccgccccggtagctgacatctcttcctatggatttccatgggtacaatgcagggtcgcgcctagcattaaagggaaattcttattattagtggttcccgcctctaataataaggatacagaaatacttgtctgtcggctactaaaagcccgagcaagggtccccccgat

SEQ ID NO: 434 - RNA, 214 nt, synthetic construct
  source 1..214: mol_type = other RNA
tatgtaatagcgcagccgttcatgttgtttacagcctctgaactgtaatgggttagtttgactgttggaagacagtcgtgctttctgaccctggtagctgctcaccccgatgctgctgtctcttgagacaggataggtgcgctcccagcaataagggcgcggatgtaccgctgtagtggctaccgaaccacccccgatcaagggggaacccgct

SEQ ID NO: 435 - RNA, 224 nt, synthetic construct
  source 1..224: mol_type = other RNA
gtttgaacagcgccgcagttcatgcttgccatcaagcctctgtgctgtgaaaaatatgggttagtttggttattggaaaatagccttgctttctgaccctagtggctgcttaccccgatgctgctgtctcttggaacaggaataaggtgcgctcccagcaaaaagggcgcgggtataccgctgtagtggctactgaatcactcccgagcaagggagaaccttct

SEQ ID NO: 436 - RNA, 262 nt, synthetic construct
  source 1..262: mol_type = other RNA
agatgaacagcgccgcagttcatgctctttgagccaatgtgctgcgataaatctgggttagtttgacggttggaaaaccgttatgctttctgaccctggtagctgcccgcttctgatgctgccatctgtagaattctatagatgggataggtgcgctcccagcaataaggagtaaggcttttagctgtagccgttgttctcaacggtgcgggttaccgcagtggtggctactgaatcacccccttcgtcgggggaaccctct

SEQ ID NO: 437 - RNA, 345 nt, synthetic construct
  source 1..345: mol_type = other RNA
aaataaatagcgccgcagttcatgctctttgagccaatgtactgtgataaatctgggttagtttgacggttggaaaaccgttttgctttctgaccctggtagctgctcgctcttgatgctgctgtctggcttgactaggcaggatatgcccttttgccatcttggataactaagtttttgatgttttcaccgacaagaaaaaacttttatacaagatatatcaaatagggacaggtgcgctcccagcaataaagagtaaagctgtaaagcttgagccgttttataacggtggggattacctcagtggcggttactgaatcacccccttcgtcgggggaaccctct

SEQ ID NO: 438 - RNA, 344 nt, synthetic construct
  source 1..344: mol_type = other RNA
aaacaaacagcgccgcagttcatgctcttcgagccaatgtactgtgataaatctgggttagtttgacggttggaaaaccgttttgctttctgaccctggtagctgcccgctcttgatgctgctgtctggcttaactaggcaggatatgcccttttggcatcttggataactaagtttttgatgtttttaccgacaataaaaaacttttatacaagtatatcaaatagggacaggtgcgctcccagcaataaagagtaaagctgcaaagcttgagccgttttataacggtggggtttacctcagtggtggctactgaatcacccccttcgtcgggggaaccctct

SEQ ID NO: 439 - RNA, 253 nt, synthetic construct
  source 1..253: mol_type = other RNA
aaagtaacagcgccgcagttcatgctcttttgagtctctgtactgtgataaatctgggttagtttgacggttgaaagaccgttttgctttctgaccctggtagctgctcgctcttgatgctgctgtcgtaagacaggataggtgcgctcccagcaataaagagtaaagctgataaagcttgagccgttgtaaaacggtggggtttacctcaataatggctactgaatcacccccttcgtcgggggaaccctcc

SEQ ID NO: 440 - RNA, 329 nt, synthetic construct
  source 1..329: mol_type = other RNA
gacttcatggcgcgctgcttcggcagctaaaaatactgggtcagtttatttgctttccgtcccaggtagttgtccgtttctggtaagtgatgtaagcgacatcctgccttgtgcaggaacatagctcacttcatagatgtacggttgcgccactttacatcaggaagcagtttttgttactagagctattaacctagcaacaaggataccgaattacaggtgctgcctgccagtaaattctttctatttaatagagaggtgcattggtaggcaggtggacagctactaaacgccccaagcaagggttgagcctaccctttttcttcatc

SEQ ID NO: 441 - RNA, 300 nt, synthetic construct
  source 1..300: mol_type = other RNA
tgttcaatagcgcgtctcgttccctgtgaacagatgataagtgtatgggcagtttaattgctttccgccctgtgtagttgtccgcgtctctttaaataattagagagacacgtcgttacggagcttgctgcgtaaatgacgttccttctgcggaacttttccgtagatagggtgcagggtcgcgcctagcatcagggagctatgtttttataaccagcggttagcgcctctggttataaggatacaggtgtacgtgtcgtggcagctaccgaatcgcctccgagcaaggaggagtcctcc

SEQ ID NO: 442 - RNA, 215 nt, synthetic construct
  source 1..215: mol_type = other RNA
taagtaatagcgccgcagttcatgttcttaggaaccgctgaactgtgaaaaatctgggttagtttgactattggaagatagtcttgctttctgaccctagtagctgctcaccccgatgctgctgtctgcggacaggaattaggtgcgctcccagcaaaaagggcgcggatatactgctgtagtggctaccaaatcaccgctccgaaaaacctatg

SEQ ID NO: 443 - RNA, 263 nt, synthetic construct
  source 1..263: mol_type = other RNA
gtattaatagcgccgcagattatctttataactgcgaaaattatgggttagtttgaccgtcggtaggcggttgtgctttctggcccttgtagctgtccaccctgatgccgatctctatacttctggaatagggatgattaacccgagagatgaggttcttagatacttcaattctatggggtaggtgcgcccccggcaataagtggcgtgggtttaccacagtgatggctataaaatcacctccgagcaagggggaatccact

SEQ ID NO: 444 - RNA, 323 nt, synthetic construct
  source 1..323: mol_type = other RNA
agccacatagttcataagctcacgcttcttggacttcctgtgttctctaaaacgggttctgttttacccttaccaagggatactttcagatccgagtagctgcaagctcatggcggagtgtcccctgacgctttgccaccgtcatagcgatgtgatggccgtctggcgtatgaacgatattgagggtgaggttgattcctaaagccgatcatacagcgcaaccaggtgaatgagtaagattccgcaaggatctaaaccttaagcagtcttctgctgaggttgggcatggttctcagtgtggctactgaccttcttgatctttt

SEQ ID NO: 445 - RNA, 341 nt, synthetic construct
  source 1..341: mol_type = other RNA
gaaacaatagcgccgcagttcaagctctttgagccgctgaactgtgaaaaatgagggtcagtttggtcgtcgcaagacaactgtgctttccgaccctggtagctgtccgctcactgactgccgtcctgacggagacttctttttgaggtttatgtggggatgggaagctgcattagttgagtccgttcttcaaatgtagcgcaggtgcgcacccagcagaagtgagtcaagcctttatcgatagggggtacaggagcatcatcacttcgatttattgatggtgatggagtgtgactgaagtggcagctactgaatcgcctccgctcaaggaggagtcctcc

SEQ ID NO: 446 - RNA, 324 nt, synthetic construct
  source 1..324: mol_type = other RNA
aaattaacagcgccgacccttcatgctcttcggagccaatgtaggtgaaaaatgggttagtttgacggttggaaaaccgttttgctttctgaccctggtagctgcccgcttctgatgctgccgtctatcgaattgctcgtccagacgggaaatcttagctctaaatatctaaatagtgtcttacttctaggatatccagagataagagaggtgcgctcccagcaataaggagtaatgcttaacttgcactagcccttggtaacaagggtgcggataaccgcagtggtggctactgaatcacccccttcatcgggggaaccctcc

SEQ ID NO: 447 - RNA, 301 nt, synthetic construct
  source 1..301: mol_type = other RNA
tattcaatagcgcgtctcgttccctgagaacagacgataagtgtaagggcagtttaactgctttccgcccttggtagttgtccgcttctctcgcttaaattggagagagacgttctttacggagcttgctctgtaagcgacgttccttttacggaagttttccgtgaatacggtgcagggtcgcgcctagcatcaaggggcaatgtttttataacagtggtaaagcacctctggttataaggatacagggttacgtgtcgtggcagctacccaatcgccttcgagcaagaaggaatcctcc

SEQ ID NO: 448 - RNA, 297 nt, synthetic construct
  source 1..297: mol_type = other RNA
atgtcaatagcgcgtcttgttccctgagaacatgacaatacagggcagtttaattgctttccgcccttggtagttgtccgcttctctcgcttaaattggagagagacgttctttacggagcttgctctgtaagtgacgttccttctacggaattttctctgtagatacggtgcagggtcgcgcctagcatcaaggggcaatgtttttataacagtggttcgcacctctggttataaggcgacaggagtacgtgtcgtggcagctacccaatcgccttcgagcaagaaggaatcctcc

SEQ ID NO: 449 - RNA, 223 nt, synthetic construct
  source 1..223: mol_type = other RNA
tatcaaatagcgccgcagatcatgcagtaaaaagcctctgaactgtgaaaaatgcgggttagtttgactgttgaaaagcagtcttgctttctgaccctggtagctgcccaccccgaagctgctgttctaaaaggacaggaattaggtgcgcccccagtaataagggtgcgggtataccgcagtggtggctactcaatcacctccgagcaaggaggaatccacc

SEQ ID NO: 450 - RNA, 304 nt, synthetic construct
  source 1..304: mol_type = other RNA
aagaaaacagcgccgcagtaacatgctcttcggagcctatgtactgcgataagtgagggtcaggtttactgttgttcagacagatgctttccgaccctggtagctgtcagcctgtcgggtcgccatctcgcagccagtgctgtatgtagatgggacacggctgtatcatgagcttgctttctcaaggtatagcataggtgcgcccccggctacgataggcaacgctctacccctggcaacagtgcggtacagatatacacttgtggcggctaccgaatcgcctccgagcaaggaggaaccctct

SEQ ID NO: 451 - RNA, 295 nt, synthetic construct
  source 1..295: mol_type = other RNA
atgtcagtagctcgtcttgttccctgagaacatgacaataaagggcagtttaattgctttccgccctggtagctgtccgcttctctcgcttaaattggagagagacgttctttatagagcttgctctgtaagtgacgttccttctgcggaacgtttccgtagatacgatgcagggttgcgcctagcatcaaggggcaatgtttttataacagtggttcacacctctggttataaagatacaggagtacgtgtcgtggcagctacccaatcgccttcgagcaagaaggaatcctcc

SEQ ID NO: 452 - RNA, 224 nt, synthetic construct
  source 1..224: mol_type = other RNA
ataatgctggcggcccagttagtgctttaggcacgaacatgtggaaaaaggtcagttttacccttgggtgctttccgacctgtgtactgtccgctattcatgctgctgcctaataaggcaattcgccacagcaatgagtagcaccgctctaccgccttaaaaaagcggtacagtaatgcagatgtggcagtccaaatcgcctactgaatacggtaggatctccc

SEQ ID NO: 453 - RNA, 220 nt, synthetic construct
  source 1..220: mol_type = other RNA
taatcaacagcgccgcaggtcatgttcatttgaaccgctgaactgcgaacagtatgggttagtttgactgttggaagacagttgtgctttctgaccctggtagctgtccactctgatgctgccgttgaaagacgggaataaggtgcgctcccagcaataggggtgtagacatactacagtgatggctaccgaatcacctccgagcaaggaggaatccact

SEQ ID NO: 454 - RNA, 267 nt, synthetic construct
  source 1..267: mol_type = other RNA
ggaacaatagcgccgcagttcaagctctttgagccgctgaactgtgaaaaatgagggtcagtttggtcgtcgtgagacaactgtgctttccgaccctggtagctgtccgctcactgactgccgtcttggcacagactccattttgaggtttgtgtggggatgggaagctgcattcgttgagtccgttcttcaaatgtagcgcaggtgcgcgcccagcagaagtgagttcagccttcactgtatgaaggtacaggagcatcatcactt

SEQ ID NO: 455 - RNA, 223 nt, synthetic construct
  source 1..223: mol_type = other RNA
tagtaaatagcgccgctggtcatgcttgcaaaagcctctgaacagtgataaatgagggttagtttgactatgggaacatggtcttgctttctggccctggtagctgcccaccccgatgctgctgtcccttgcggacaggaattaggtgcgcccccagtaataagggtgcgggtttaccgcagtggtggctactcaatcacctccgaccaaggaggaacccacc

SEQ ID NO: 456 - RNA, 305 nt, synthetic construct
  source 1..305: mol_type = other RNA
tattcaatagcgcgtctcgttccccgtgaacagatgataagtgtcggggcagtttaattgctttccgcccttggtagttgtccgcgtctctccaatctgtagagaagagagacacgtcgggagcagagcttgctctgtaatcgacgttccttctacgaaactctttcgtagatatggtgcagggtcgcgcctagcatcagggagctatgtttttataaccttggtttagagcctctggttataaggatacaggtttacgtgttgtggcagctaccgaatcgccttcgagcaggaaggaccttccc

SEQ ID NO: 457 - RNA, 238 nt, synthetic construct
  source 1..238: mol_type = other RNA
tattcaatagcgcaccgttctctaagaacaggtgacaattaaggggcagtttaattgctttccgtcccaggtagttgtcctctcttttcataggcttacctatgaatgggtgcagggtcgcgcctagcatcagagagctatgttttcatagggtggttaagcgccactactatgaggatacaggaatacgtgtaatcgggctgctaccaaaccgcctccgagcaaggaggaacccctt

SEQ ID NO: 458 - RNA, 226 nt, synthetic construct
  source 1..226: mol_type = other RNA
gtgaaaatagcgccgcagtttaagctcagcaagcctctggactgcgaaaagtatggggtagtttgaccgtcggtaaacggttgtgctttctgcccctggcgactgcccaccccgatgctgtcgatttcttaactgggaatcgagatgaggtgcgcccccagcaagagggaacgggtttactggagtggtggtcgccgaatcacccccgagcaagggggactcgtcc

SEQ ID NO: 459 - RNA, 223 nt, synthetic construct
  source 1..223: mol_type = other RNA
taattaatagcgccgccggtcatgcttgcaagaacctctgatcggtgataaatgagggttagtttgacggttggaagaccgttgtgctttctgaccctggtagctgcccaccccgaagctgctatcccttggggataggaattaggtgcgcccccagtaataagggtgcgggtttaccgcagtggtggctactgaatcacctccgaccaagggggaacccact

SEQ ID NO: 460 - RNA, 299 nt, synthetic construct
  source 1..299: mol_type = other RNA
agcgtcgcagtccatgctttttaatcaagcctctgcactgtgaaaaaattgggttagtttgactgtctggagatagtccttctttctgaccctggtaactacccgcaactgaagctgctatctctaagtctcagctaggagataggacatacttaaaataaagaacatcgtatctttatcttattaggttaggtgcgctcccagcaattaagttgtatagttttaagactgggaaatatcttaaaacttaatcctgctagttcaggatgtagatgactacagtggcggttactgaatca

SEQ ID NO: 461 - DNA, 576 bp, synthetic construct
  source 1..576: mol_type = other DNA
attcaatatttgagctttttgcaaaaataaacagatatcaaagttagtttgctcgagcgaaattaattgttattagctaaatataatttttcccctcatcaagagatgcgatcactaccttagtaacttgcttgctaacctctgataaataacatccaaccatacaggaatcacagctttaccttcatccagggatgcgatcgcgaatcgctccgcaggaatcgctcttttcctgtcaacttatactaactaatgtatgatctaaattactcttcactgtcctttagtaatcagtgaatctacctgtcaacgtgtacgttcgcacattatatgtcgtatttcgcaagtcatgtcgcaactgcttttaaggactaaaacctttattctataagaatcataagctatttacccctacaaaagacaaatttatataattcgcatattatatgtcgcaaaacttgatttcgcaaattaaacgtcgtttattaagattttggtattttgcaaattagatgtcgcatttttgaggaattcatggtacattagtaccttaattattcatgagtgggtttcact

SEQ ID NO: 462 - DNA, 418 bp, synthetic construct
  source 1..418: mol_type = other DNA
tgcaacaaacttttttgttagtcatataatgggaggattgaaaggagcaactgattcgtaatcaggaattaaagtaaaatgtttatgcaacccggattattaagtatgagcatctaacgaataatgataaataccgtagtgcaatattatgctacacgaaacttaatcagtgtttacataattggcgacattaatttgcgaaattatattaactatctatttaatcaataaaatgttgatgagcgacattaatttgcgaagaacgacattaatttgcgaaatgcgacatataatttgcgaatgtacatactaaagccgatgatgggatttgaacccacgacctactgattacgaatcagttgctctacccctgagccacatcggcatacacagcttaacattatagcattatttacct

SEQ ID NO: 463 - DNA, 652 bp, synthetic construct
  source 1..652: mol_type = other DNA
gtttagcttattgggggaatactaagaggatgtctgtctcaaaagtgtcattttgtcattctcagtagagcgaagtgagactagtactgttttgagggaaacgtcggtggaagcaactggtgagtccagtcctgcaccgacgttttccgcatcttgggatttaactttcccgttgggcggaacgaggagttcctttttaccgtcaaactttgagatactgatacgtgtgttgcattcatacttgtcaccttaccctgccctcacgggcacttttctccaaggcatgccaagggggcacttggtgagatgatttcgccgcaacttggtatgagcgtccaaggtgcacactacccgttcatgtacattcacaaattaaatgtcgctatttcacaaattaatgtcgtttttttattcataaaacccttaccatgcaagggttttatttttaaggtgattttggctactcttttataatttaacatattataagtcgttaacactattttttcactaattaaacgtcgcttatttggataagcggaaaatacacttaattatttttcataaattaaatgtcgtaaagtgagacgtttgatagtacattagttttattaagagcgagaaatcctccacttattgctgtgaggtgaaa

SEQ ID NO: 464 - DNA, 712 bp, synthetic construct
  source 1..712: mol_type = other DNA
gcttgttgctctgcaaagaaccttctgtctttctgttcctatacccagccgtgtcaaaaaatcttgcctgaaaactgcaccagatattttatatctagcccataatttcaaggtagctttggtatatcactagtttatcaatatcgtcagtgaaaagaatcacaaatttgtttgattttcattacacccacagcgcctgaaagtatgtttataggttgctaaatcggcagtatgatcaaaatgagatattgagagcgcgatttggacaaatataactcttcacactagtatattgtcaaatcagcttgcttatggtacaatgacaaattagtgtgatgttcaaaaaagccgaatatgccagcatagcacagtggtagtgcatccgacttgtaatcggaaggtcgtcggttcaaatccgactgctggctttttacttcccatatagcgctgtgttgtacaggtgtacattcacaaattaaatgtcgctgttcacaaattaacgtcgcatttcacaaattgatgtcgtatttcacaaattaacgtcgcatatgcattctctgtagccgttaacaaattaaatgtcgcttactttgattttgatttagctgtacaaggattgctatagcaaagttgctacaagcaaagttagtacgtagcctaatattttaactttacataagctcttccaaacaaagcagacggatttgaaccc

SEQ ID NO: 465 - DNA, 299 bp, synthetic construct
  source 1..299: mol_type = other DNA
atgtattgtcagaatttggtgttgtacattaactaattatttgtcaatttaacaaattattgtcactactctatcaaatgttgaaagccctgtcatggaaaggtttctgacggggtttttattattttcacattcttaacccttcaaagcttgattaacatattagctgtcaaatgccagaattataacacattaattgtcattttccaaaaaacaaacaaaccaatgattttgcaaaatttaacaaattctttgtccagatgtcagtataatacaactatgttttcataaaacacaaa

SEQ ID NO: 466 - DNA, 453 bp, synthetic construct
  source 1..453: mol_type = other DNA
ttttgctggcagtgagatactttcaatattttgatgttttaagccttagctaaaactaaatggattaatcaagagtggaaagaataatttaaaattattagtggtgggttgaaagcgaatccagttacgtccttattaactggttagtgaacttagaaagcaaacaaatcaatttcagttaaaatcaagaggcaaattttagtgactagctttaggacactaatctgtcaaaagtgacattaatttgtcaacgcggtcaaataatttggcaatcgacaacactaaattatttagccaacaaaggtgtcatcggtataaatttatggacgtaactggattcgaaccagtgaccccatccatgtcaaggatgtactctaaccaactgagctatacgtccgaatttttcgcaacttgttaagtatagcatagcttattctacaaggcaagatatat

SEQ ID NO: 467 - DNA, 520 bp, synthetic construct
  source 1..520: mol_type = other DNA
aaatctaccccaaagcttttaagggactgttttttatagatgtcacccaatttccaacggacgaacgtgcaatactcaactgagcagcttttctcaaattttaaatggtagctaaaactgtgactaaatttgtgactaaaatcgtgggttgattctaccggatttgaactaaaatggcacaaacgaagaatccactcctcgaagctagaaggtgcttgtggagcgggaaacaacggaatataaactgtacattaactaattatttgtcaatttaacaaaataattgtcatattattcaaaatcgctcaacccctgtcattgcaataactatggcagggattttgctattaacagcagtcagactgctttcaactttgattcacaaattagatgtcaaaatccagatttttcacaatttaattgtcaaatcccaaaaatagtggaaaatcatgattttgcataaattaacaaattagttgtcactcagatagtataatacaactatgttttgataaaacac

SEQ ID NO: 468 - DNA, 999 bp, synthetic construct
  source 1..999: mol_type = other DNA
ctttgctccatatgtatagcgagattagaactttatttatggtgggttgagaggtaaggcagaggtctgccattattggtgtaaggacaagaatccttagttatgaaaagccatataggttttctaaaagaatgcggacattaatttgtcaaaagcaacttctaccattaaaagttaaatatcaaggacacataatctgttaacagtgacatcaattcgttaaaagtgacaccaatctgttaacagtgacaaataatgcgtgaatgtacaataaacaaaggcgacacccggatttgaaccgggggatggaggttttgcagacctctgccttaccacttggctatgtcgccgcactatatatttattattttaccaatatttgatataaaaatctacccctcctaatttatttgattaaatatccttaaagcattgagcagccagagaaaaggccatcgaaggaaaaacatagctgctaaatattgaatacttcacgaagaaaactatgttttctcataacagtctaaaaaggagtatgactctacaattgccattccaacttacggtttatccataaataggaagcgaggacatctaaaactcatcccgacaattttttcgttttccttctacccattgcattgcctcacatttgtgctaggattttcggttacgtagaatacatcttttttttcataaaaacagtatttatactttttaagatataagtactgcgaaacatgaaaaccaaatttttatatcttaggatagatgaaaaacataaaaaaaaataaaattagtataagatatggaagctaattctcggtttatacttaaaaaattgagaaataagtaaacatacataaccttaaaaatatcgaagactcaacagtattaatatgctactaaagaagcattaactaccagtatacaaaaggcgttatgactacaaaggagaattggatattaatactcgtattagattggtatcaccaaaga

SEQ ID NO: 469 - DNA, 35 bp, synthetic construct
  source 1..35: mol_type = other DNA
ttgacagctagctcagtcctaggtataatgctagc

SEQ ID NO: 470 - DNA, 551 bp, synthetic construct
  source 1..551: mol_type = other DNA
cgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataata

SEQ ID NO: 471 - protein, 16 aa, synthetic construct
KRPAATKKAGQAKKKK

SEQ ID NO: 472 - protein, 19 aa, synthetic construct
PKKKRKVDGSPKKKRKVDS

SEQ ID NO: 473 - RNA, 173 nt, synthetic construct
  source 1..173: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggt

SEQ ID NO: 474 - RNA, 68 nt, synthetic construct
  source 1..68: mol_type = other RNA
gttaccacagtggtggctactgaatcacctccgagcaaggaggaacccactgaaaggtgggttgaaag

SEQ ID NO: 475 - RNA, 180 nt, synthetic construct
  source 1..180: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccac

SEQ ID NO: 476 - RNA, 61 nt, synthetic construct
  source 1..61: mol_type = other RNA
gagtggtggctactgaatcacctccgagcaaggaggaacccactgaaaggtgggttgaaag

SEQ ID NO: 477 - RNA, 104 nt, synthetic construct
  source 1..104: mol_type = other RNA
gaattaatagcgccgccgttcatgcttctaggagcctctgaaaggtgacaaatgcgggttagtttggctgttgtcagacagtcttgctttctgaccctggtagc

SEQ ID NO: 478 - RNA, 137 nt, synthetic construct
  source 1..137: mol_type = other RNA
gtgcccaccccgaagctgctgttccttgtgaacaggaattaggtgcgcccccagtaataagggtatgggtttaccacagtggtggctactgaatcacctccgagcaaggaggaacccactgaaaggtgggttgaaag

SEQ ID NO: 479 - RNA, 115 nt, synthetic construct
  source 1..115: mol_type = other RNA
gaattaatagcgccgccgttcatgcttctaggagcctctgaaaggtgacaaatgcgggttagtttggctgttgtcagacagtcttgctttctgaccctggtagctgcccaccccg

SEQ ID NO: 480 - RNA, 126 nt, synthetic construct
  source 1..126: mol_type = other RNA
gaagctgctgttccttgtgaacaggaattaggtgcgcccccagtaataagggtatgggtttaccacagtggtggctactgaatcacctccgagcaaggaggaacccactgaaaggtgggttgaaag

SEQ ID NO: 481 - RNA, 129 nt, synthetic construct
  source 1..129: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttcct

SEQ ID NO: 482 - RNA, 112 nt, synthetic construct
  source 1..112: mol_type = other RNA
gtgtgaacaggaattaggtgcgcccccagtaataagggtatgggtttaccacagtggtggctactgaatcacctccgagcaaggaggaacccactgaaaggtgggttgaaag

SEQ ID NO: 483 - RNA, 132 nt, synthetic construct
  source 1..132: mol_type = other RNA
aattaatagcgccgccgttcatgcttctaggagcctctgaaaggtgacaaatgcgggttagtttggctgttgtcagacagtcttgctttctgaccctggtagctgcccaccccgaagctgctgttcggtcgg

SEQ ID NO: 484 - RNA, 137 nt, synthetic construct
  source 1..137: mol_type = other RNA
ccgaccgaacaggaattaggtgcgcccccagtaataagggtatgggtttaccacagtggtggctactgaatcacctccgagcaaggaggaacccactgaaaggtgggttgaaagnnnnnnnnnnnnnnnnnnnnnnn

SEQ ID NO: 485 - RNA, 204 nt, synthetic construct
  source 1..204: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcagaaag

SEQ ID NO: 486 - RNA, 194 nt, synthetic construct
  source 1..194: mol_type = other RNA
gaattaatagcgccgccgttcatgcttctaggagcctctgaaaggtgacaaatgcgggttagtttggctgttgtcagacagtcttgctttctgaccctggtagctgcccacccgaaagggtatgggtttaccacagtggtggctactgaatcacctccgagcaaggaggaacccactgaaaggtgggttgaaag

SEQ ID NO: 487 - RNA, 219 nt, synthetic construct
  source 1..219: mol_type = other RNA
gcgccgccgttcagaaatgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaag

SEQ ID NO: 488 - RNA, 224 nt, synthetic construct
  source 1..224: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacagcagtaataagggtatgggtttaccacagtggtggctactgaatcacctccgagcaaggaggaacccactgaaaggtgggttgaaag

SEQ ID NO: 489 - RNA, 234 nt, synthetic construct
  source 1..234: mol_type = other RNA
gaattaatagcgccgccgttcatgcttctaggagcctctgaaaggtgacaaatgcgggttagttttgttagacagtcttgctttctgaccctggtagctgcccaccccgaagctgctgttccttgtgaacaggaattaggtgcgcccccagtaataagggtatgggtttaccacagtggtggctactgaatcacctccgagcaaggaggaacccactgaaaggtgggttgaaag

SEQ ID NO: 490 - RNA, 221 nt, synthetic construct
  source 1..221: mol_type = other RNA
gaattaatagcgccgccgttcatgcttctaggagcctctgaaaggtgacaaatgcgggttagtttggctgttgtcagacagtcttgctttctgaccctggtagctgcccaccccgaagctgctgttccttgtgaacaggaattaggtgcgcccccagtaataagggtatgggtttaccacagtggtggctactgaatcacctccgagcaaggaggagaaag

SEQ ID NO: 491 - RNA, 261 nt, synthetic construct
  source 1..261: mol_type = other RNA
aataatagcgccgcagttcatgttcttaggaaccgctgaactgtgaaaaatctgggttagtttgactattggaagatagtcttgctttctgaccctagtagctgctcaccccgatgctgctgtctgcggacaggaattaggtgcgctcccagcaaaaagggcgcggatatactgctgtagtggctaccaaatcaccctcgaccaagggggaacccatcccgaaagggcgggttgaaagnnnnnnnnnnnnnnnnnnnnnnn

SEQ ID NO: 492 - RNA, 252 nt, synthetic construct
  source 1..252: mol_type = other RNA
taatagcgcagccgttcatgttgtttacagcctctgaactgtaatgggttagtttgactgttggaagacagtcgtgctttctgaccctggtagctgctcaccccgatgctgctgtctcttgagacaggataggtgcgctcccagcaataagggcgcggatgtaccgctgtagtggctaccgaaccacccccgatcaagggggaacccgcttgaaagggcaggttgaaagnnnnnnnnnnnnnnnnnnnnnnn

SEQ ID NO: 493 - RNA, 255 nt, synthetic construct
  source 1..255: mol_type = other RNA
aatagcgccgtagttcatgcttgctaaagcctctgaattgcgaaaagtccgggttagtgctgtcggcagacagcgttgctttctgaccctggtagctgcccaccccgatgctgctgtcccttgcagacaggaaccaggtgcgcccccagtaataagggtgtgggtttaccacagtggtggctactgaatcacctccgagcaaggaggaatccaccgaaaggtgggttgaaagnnnnnnnnnnnnnnnnnnnnnnn

SEQ ID NO: 494 - protein, 89 aa, unidentified
  source 1..89: note = MG190-1 ribosomal protein S15
MALLQQRKQEIISDYQVHETDTGSSDVQVAMLTERINKLSAHLKGNKKDHASRRGLLKMIGQRKRLLAYILRQDKDRYRALITRLGIRG

SEQ ID NO: 495 - protein, 89 aa, unidentified
  source 1..89: note = MG190-2 ribosomal protein S15
MALLQQRKQEIITDYQVHETDTGSSDVQVAMLTERINKLSSHLKGNKKDHASRRGLLKMIGQRKRLLAYIMRQDKDRYRALITRLGIRG

SEQ ID NO: 496 - protein, 89 aa, unidentified
  source 1..89: note = MG190-3 ribosomal protein S15
MPLQQERKQTVINDFQTHGTDTGSADVQVALLTARVEQLSEHLKKNKKDHASRRGLLQIIGRRKRLLAYILKQDRERYQALIKKLGIRG

SEQ ID NO: 497 - protein, 89 aa, unidentified
  source 1..89: note = MG190-4 ribosomal protein S15
MPLQQERKQTAINEYQTHSTDTGSAEVQVALLTARVEQLSEHLKKNKKDHSSRRGLLQIIGRRKRLLAYILKNDREHYQALIKKLGIRG

SEQ ID NO: 498 - protein, 89 aa, unidentified
  source 1..89: note = MG190-5 ribosomal protein S15
MPLQQERKQTVINDFQTHGTDTGSADVQVALLTARVEQLSEHLKKNKKDHASRRGLLMIIGRRKRLLAYILKEDRARYQALIKKLGIRG

SEQ ID NO: 499 - protein, 89 aa, unidentified
  source 1..89: note = MG190-6 ribosomal protein S15
MPLKQEIKQKLINEYQAHPTDTGSAELQVAMLTARVQQLSEHLKANKKDHASRRGLLKIIGRRKRLLAYILKRDRDAYQALIQKLGIRG

SEQ ID NO: 500 - protein, 89 aa, unidentified
  source 1..89: note = MG190-7 ribosomal protein S15
MALTQERKQQLITEYQVHETDTGSTNVQIAILTDRINKLSEHLKTNKNDHSSRRGLLKLIGQRKRLLSYISKENKERYQALIGRLGIRG

SEQ ID NO: 501 - protein, 89 aa, unidentified
  source 1..89: note = MG190-8 ribosomal protein S15
MALTQERKQEIMGQYQAHETDTGSADLQVAMLSDRINKLSAHLKVNQKDFSSRRGLMQLIGRRRRLLSYIQKQDRARYQALIARLGIRG

SEQ ID NO: 502 - protein, 89 aa, unidentified
  source 1..89: note = MG190-9 ribosomal protein S15
MSLTQQRKQEIMTEYQVHETDTGSAEVQVAMMTERINRLSAHLKANHKDHASRRGLLTIIGQRKRLLAYIQKKDQQNYQALIGRLGIRG

SEQ ID NO: 503 - protein, 105 aa, unidentified
  source 1..105: note = MG190-10 ribosomal protein S15
MRDCCINLEKSEIESIMALTQQHKQEIISNYQVHETDTGSADVQIAMLTERINRLSQHLQANKKDHSSRRGLLKLIGQRKRLLSYVQQESREKYQALIGRLGIRG

SEQ ID NO: 504 - protein, 89 aa, unidentified
  source 1..89: note = MG190-11 ribosomal protein S15
MALTQERKQEILTQYQVHETDTGSADVQVAMLTARIIRLSEHLQANKKDHSSRRGLLKLIGQRKRLLSYILEENRERYQALIGRLGIRG

SEQ ID NO: 505 - protein, 89 aa, unidentified
  source 1..89: note = MG190-12 ribosomal protein S15
MALTQQRKQEIITQYQVHETDTGSSDVQVAMLTARIMRLSEHLQGNKKDHSSRRGLLKLIGQRKRLLSYIMQEDRERYQALIARLGIRG

SEQ ID NO: 506 - protein, 89 aa, unidentified
  source 1..89: note = MG190-13 ribosomal protein S15
MALTQERKQEIIVNYQVHETDTGSAEVQVAMLTERINRLSLHLQANKKDHSSRRGLLKLIGQRKRLLAYILKDSREKYQALIGRLGIRG

SEQ ID NO: 507 - protein, 89 aa, unidentified
  source 1..89: note = MG190-14 ribosomal protein S15
MTLTQQRKQELITQYQVHETDTGSADLQVAMLTERINRLSQHLQANKKDHSSRRGLLKLIGQRKRLLSYIQEGSRERYQALIARLGIRG

SEQ ID NO: 508 - protein, 89 aa, unidentified
  source 1..89: note = MG190-15 ribosomal protein S15
MTLTQERKHEIIDGYQVHDTDTGSVDVQVAVLTERINRLSNHLKTNKKDHSSRRGLLQMIGRRKRLLSYLRKEDIARYQNLIQRLGIRG

SEQ ID NO: 509 - protein, 89 aa, unidentified
  source 1..89: note = MG190-16 ribosomal protein S15
MTLTQQRKQELITQYQVHETDTGSAEVQVAMLTERINRLSQHLQTNKKDHSSRRGLLKLIGQRKRLLSYIQEGSRERYQALIGRLGIRG

SEQ ID NO: 510 - protein, 89 aa, unidentified
  source 1..89: note = MG190-17 ribosomal protein S15
MPLLQQRKQELISDYQVHETDTGSADVQVAMLTERINRLSEHLKTNKKDHASRKGLLRMIGLRKRLLSYIQKQDNARYRALITRLGIRG

SEQ ID NO: 511 - protein, 91 aa, unidentified
  source 1..91: note = MG190-18 ribosomal protein S15
MTLTQARKQEIMSAHQLHATDTGSADLQVAMLTERITRLSEHLKANKSDHASRRGLLKMIGQRKRLLAFINTESVQRYQSLADSLGIRRVK

SEQ ID NO: 512 - protein, 91 aa, unidentified
  source 1..91: note = MG190-19 ribosomal protein S15
MSLTQERKQEIMSAHQLHATDTGSSDLQVAMLTERINRLSEHLKANKSDHASRRGLLKMIGQRKRLLAFINTESVQRYQNLADSLGIRRVK

SEQ ID NO: 513 - protein, 89 aa, unidentified
  source 1..89: note = MG190-20 ribosomal protein S15
MALTQERKQEIMGDYQTHETDTGSPDVQVAMLTDRITKLSAHLKINQKDFASRRGLMMMISRRKRLLAYIQKENVDRYKALIARLGIRG

SEQ ID NO: 514 - protein, 89 aa, unidentified
  source 1..89: note = MG190-21 ribosomal protein S15
MALTQERKQEIMGDFQTHETDTGSADVQIAMLSDRISKLSAHLKINQKDFASRRGLMMMISRRKRLLAYLQKENVDRYKALIARLGIRG

SEQ ID NO: 515 - protein, 89 aa, unidentified
  source 1..89: note = MG190-22 ribosomal protein S15
MSLVQEDKQKIITDFQKHETDTGSVEVQVAMLTERINRLSGHLKTNKKDHGSRIGLLKMISLRKRLLSYVQKLDFARYKTLIGRLGIRG

SEQ ID NO: 516 - protein, 89 aa, unidentified
  source 1..89: note = MG190-23 ribosomal protein S15
MALLQERKQEIISEYQVHETDTGSADVQVAMLTERINRLSAHLKGNKKDHASRRGLLKMIGQRKRLLAYILKQDQERYRALVTRLGIRG

SEQ ID NO: 517 - protein, 89 aa, unidentified
  source 1..89: note = MG190-24 ribosomal protein S15
MTLTQEQKQEIINTHQVHATDTGSADVQVAMLTDRISRLSLHLQANKKDYASRQGLLQMIGQRKRLLGYINKRSPEKYRALITKLGIRG

SEQ ID NO: 518 - protein, 89 aa, unidentified
  source 1..89: note = MG190-25 ribosomal protein S15
MALLQAQKQEIISGYQTHETDTGSADVQIAILTEKINRLSLHLRTNKKDHASRQGLLRMISKRKSLLAYLISEDQTRYRALVTRLGIRG

SEQ ID NO: 519 - protein, 89 aa, unidentified
  source 1..89: note = MG190-26 ribosomal protein S15
MALLQERKQELISEYQIHETDTGSSEVQVAMLTERINKLSAHLKTNKKDHASRRGLLKMIGQRKRLLAYIQKKSQDEYRALITRLGIRG

SEQ ID NO: 520 - protein, 89 aa, unidentified
  source 1..89: note = MG190-27 ribosomal protein S15
MSLLQAQKQEIITNYQTHETDTGSADVQIAILTEKVSRLSLHLRTNKKDHASRQGLLRMISQRKRLLAYVLKHDPERYRALVTRLGLRG

SEQ ID NO: 521 - protein, 89 aa, unidentified
  source 1..89: note = MG190-28 ribosomal protein S15
MTLLQERKQEIISDYQVHETDTGSADVQVAILTERINRLSAHLKGNKKDHASRRGLLKMIGQRKRLLAYIMKQDQTRYRALIGRLGIRG

SEQ ID NO: 522 - protein, 89 aa, unidentified
  source 1..89: note = MG190-29 ribosomal protein S15
MTLLQERKQEIINDFQKHGTDTGSADVQVAMLTERINKLSAHLRENKKDHASRRGLLKMIGQRKRLLAYIQAADQDRYRALITRLGIRG

SEQ ID NO: 523 - protein, 89 aa, unidentified
  source 1..89: note = MG190-30 ribosomal protein S15
MTLLQERKQEIINDFQKHGTDTGSADVQVAMLTERINKLSAHLRENKKDHASRRGLLKMIGQRKRLLAYILKEDQGRYRALITRLGIRG

SEQ ID NO: 524 - protein, 89 aa, unidentified
  source 1..89: note = MG190-31 ribosomal protein S15
MTLLQERKQEIINDFQKHGTDTGSADVQVAMLTERINKLSAHLRENKKDHASRRGLLKMIGQRKRLLAYIIKEDQDRYRALITRLGIRG

SEQ ID NO: 525 - protein, 89 aa, unidentified
  source 1..89: note = MG190-32 ribosomal protein S15
MTLTQSKKQELITQYQTHETDTGSADLQVAILTERINQLTGHLQANPKDHASRRGLLQMIGRRRGLLKYIQQKDQGRYQALIGKLGIRR

SEQ ID NO: 526 - protein, 89 aa, unidentified
  source 1..89: note = MG190-33 ribosomal protein S15
MALTQQRKQELITQYQVHETDTGSSDVQVAMLTARIMRLSEHLQGNKKDHSSRRGLLKLIGQRKRLLSYIMQEDRERYQALIARLGIRG

SEQ ID NO: 527 - protein, 89 aa, unidentified
  source 1..89: note = MG190-34 ribosomal protein S15
MTLLQERKQEIINEYQTHETDTGSADVQVAMLTERINKLSAHLRENKKDHASRRGLLKMIGQRKRLLAYILKQDQDRYRALITRLGIRG

SEQ ID NO: 528 - protein, 89 aa, unidentified
  source 1..89: note = MG190-35 ribosomal protein S15
MALVQERKQEIIGQYQVHETDTGSADVQVAILTERINKLSLHLRSNKKDHASRTGLLKMIGQRKRLLAYIQKGDVDRYRALIGRLGIRG

SEQ ID NO: 529 - protein, 89 aa, unidentified
  source 1..89: note = MG190-36 ribosomal protein S15
MTLLQERKQEIINGFQKHGTDTGSADVQVAMLTERINKLSAHLRENKKDHASRRGLLKMIGQRKRLLAYIIKEDQDRYRALITRLGIRG

SEQ ID NO: 530 - protein, 89 aa, unidentified
  source 1..89: note = MG190-37 ribosomal protein S15
MTLLQERKQEIISDYQVHETDTGSADVQVAILTERINRLSAHLKINKKDHASRRGLLKMIGQRKRLLAYILKQDQERYRALIGRLGIRG

SEQ ID NO: 531 - protein, 89 aa, unidentified
  source 1..89: note = MG190-38 ribosomal protein S15
MTLLQERKQEIINDFQKHGTDTGSADVQVAMLTERINKLSAHLRENKKDHASRRGLLKMIGQRKRLLAYILKEDQDRYRALITRLGIRG

SEQ ID NO: 532 - protein, 89 aa, unidentified
  source 1..89: note = MG190-39 ribosomal protein S15
MALTQQRKQELISDYQVHETDTGSSEVQIAMLTERINRLSEHLKANQQDHSSRRGLLKIIGQRKQLLAYVQKSNKEKYQALIARLGIRG

SEQ ID NO: 533 - protein, 89 aa, unidentified
  source 1..89: note = MG190-40 ribosomal protein S15
MALTQQRKQELITGYQVHETDTGSSEVQIAMLTDRITRLSEHLRANQQDHSSRRGLLKIIGQRKRLLAYVQKQNREKYQALIARLGIRG

SEQ ID NO: 534 - protein, 89 aa, unidentified
  source 1..89: note = MG190-41 ribosomal protein S15
MPLLQARKQEIIGEYQVHETDTGSSEVQVAMLTDRIIKLSAHLKTNKKDHASRRGLLKMIGQRKRLLAYINKKDPNNYRQLITRLGIRG

SEQ ID NO: 535 - protein, 89 aa, unidentified
  source 1..89: note = MG190-42 ribosomal protein S15
MALLQERKQEVISEYQVHETDTGSADVQVAMLTERINRLSAHLKGNKKDHASRRGLLKMIGQRKRLLAYILKQDQERYRALVTRLGIRG

SEQ ID NO: 536 - protein, 89 aa, unidentified
  source 1..89: note = MG190-43 ribosomal protein S15
MTLLQERKQEIISGYQVHETDTGSADVQVAILTERINRLSAHLKINKKDHASRRGLLKMIGQRKRLLAYILKQDQERYRALIGKLGIRG

SEQ ID NO: 537 - protein, 110 aa, unidentified
  source 1..110: note = MG190-44 ribosomal protein S15
MVVTCTVGPQPGPSLRFTPQPMPLTTTKKQELINGHQTHGTDTGSVEVQVAMLSERISQLTGHLQKNKHDFSSRQGLLKMIGRRKRLLSYLNGISKERYSALIAKLGIRG

SEQ ID NO: 538 - protein, 89 aa, unidentified
  source 1..89: note = MG190-45 ribosomal protein S15
MALTQQRKQELISGYQVHETDTGSADVQIAMLTDRINRLSQHLQANKKDHSSRRGLLKMIGQRKRLLSYIQQDSREKYQALIARLGIRG

SEQ ID NO: 539 - protein, 89 aa, unidentified
  source 1..89: note = MG190-46 ribosomal protein S15
MPLDTTKKQELINSHQTHGTDTGSVEVQVAMLSERVSQLTGHLQQNKHDFSSRQGLLKMIGRRKRLLGYLRAQSEDRYAQLIAKLGIRG

SEQ ID NO: 540 - protein, 89 aa, unidentified
  source 1..89: note = MG190-47 ribosomal protein S15
MALVQERKQEIISQYQVHETDTGSADVQVAMLTERINKLSLHLRSNKKDHASRTGLLKMIGQRKRLLAYIQKGDTDRYRALITRLGIRG

SEQ ID NO: 541 - protein, 89 aa, unidentified
  source 1..89: note = MG190-48 ribosomal protein S15
MALVQERKQEIITQYQVHETDTGSADVQVAILTERINKLSLHLRSNKKDHASRTGLLKMIGQRKRLLAYIQKGDAAHYRELIARLGIRG

SEQ ID NO: 542 - protein, 89 aa, unidentified
  source 1..89: note = MG190-49 ribosomal protein S15
MALLQQRKQEIISDYQVHETDTGSSEVQVAMLTERINKLSLHLRTNKKDHASRMGLLKMIGQRKRLLAYINKRSPEKYRALITRLGIRG

SEQ ID NO: 543 - protein, 89 aa, unidentified
  source 1..89: note = MG190-50 ribosomal protein S15
MALTQQRKQELISEYQVHDTDTGSTEVQVAMLTERISRLSEHLRSNQKDHSSRRGLLKLIGQRKRLLSFLQSEDKQKYQNLLTRLGIRG

SEQ ID NO: 544 - protein, 89 aa, unidentified
  source 1..89: note = MG190-51 ribosomal protein S15
MALTQQRKQELISEYQVHDTDTGSTEVQIAMLTERINRLSEHLKGNQKDFSSRRGLLKLIGQRKRLLSYLQSENRERYQTLISRLSIRG

SEQ ID NO: 545 - protein, 89 aa, unidentified
  source 1..89: note = MG190-52 ribosomal protein S15
MTLTQQRKHELINEHQVHETDTGSADVQIAMLTERINRLSAHLKSNKSDHASRRGLLTIIGRRKRLLAYVQKEDLSRYQALIAKLGIRG

SEQ ID NO: 546 - protein, 89 aa, unidentified
  source 1..89: note = MG190-53 ribosomal protein S15
MSLTQERKHEIIEGYQVHETDTGSAEVQIAMLTERINRLSEHLKANSKDHSSRRGLLQLIGRRKRLLAYMRRESAERYPALIQRLGIRG

SEQ ID NO: 547 - protein, 89 aa, unidentified
  source 1..89: note = MG190-54 ribosomal protein S15
MALTQERKQEIIVNYQVHETDTGSADVQIAMLTERINRLSLHLQANKKDHSSRRGLLKLIGQRKRLLAYIQKDSREKYQALIGRLGIRG

SEQ ID NO: 548 - protein, 104 aa, unidentified
  source 1..104: note = MG190-55 ribosomal protein S15
MITWEIAGLHELTPSMSLTQERKHELINGYQVHETDTGSADVQIAMLTERINRLSDHLKNNKKDHSSRRGLLTMIGQRKRLLAFLRKENTERYQSLIQRLGIRG

SEQ ID NO: 549 - protein, 89 aa, unidentified
  source 1..89: note = MG190-56 ribosomal protein S15
MSLTQERKHEIIDGYQVHETDTGSADVQIAMLTERINRLSEHLKNNKKDHSSRRGLLQMIGRRKRLLSYLRNENKERYQALIQRLGIRG

SEQ ID NO: 550 - protein, 89 aa, unidentified
  source 1..89: note = MG190-57 ribosomal protein S15
MTLTQERKQEILQGYQVHETDTGSADVQIAMLTERINRLSEHLKTNKKDHSSRRGLLKMIGQRKRLLGYLRKENIERYQALIQRLGIRG

SEQ ID NO: 551 - protein, 89 aa, unidentified
  source 1..89: note = MG190-58 ribosomal protein S15
MTLTQERKHEIIEGYQVHETDTGSADVQIAMLTERINRLSEHLKSNKKDHSSRRGLLKMIGLRKRLLSYLRKEDTARYQALIQRLGIRG

SEQ ID NO: 552 - protein, 89 aa, unidentified
  source 1..89: note = MG190-59 ribosomal protein S15
MSLTQERKHEIIEGYQVHETDTGSADVQIAILTERINRLSEHLRANSKDHASRRGLLQLIGRRKRLLAYMRKGDMSHYQALIQRLGIRG

SEQ ID NO: 553 - protein, 107 aa, unidentified
  source 1..107: note = MG190-60 ribosomal protein S15
MLRVITWETAGLYELTLSMTLTQERKHEIIDGYQVHDTDTGSVDVQVAMLTDRINRLSNHLKTNKKDHSSRRGLLQMIGRRKRLLSYLRKEDIQRYQNLIQRLGIRG

SEQ ID NO: 554 - protein, 89 aa, unidentified
  source 1..89: note = MG190-61 ribosomal protein S15
MALTQQRKQELISEYQVHDTDTGSSEVQIAMLTERINRLSEHLRGNQKDYSSRRGLLKLIGQRKCLLSYVQAEDRQKYQALIARLGIRG

SEQ ID NO: 555 - protein, 89 aa, unidentified
  source 1..89: note = MG190-62 ribosomal protein S15
MALLQERKQEIITDYQIHETDTGSADIQVAMLTERINKLSAHLRENKKDHSSRRGLLKMIGQRKRLLAYILKHDQERYRGLLTRLGIRG

SEQ ID NO: 556 - protein, 89 aa, unidentified
  source 1..89: note = MG190-63 ribosomal protein S15
MSLTQERKHEIIDGFQVHETDTGSAEVQIAMLTERINRLSEHLKINKKDHSSRRGLLQMIGRRKRLLSYIRNENVQRYQALIQRLGIRG

SEQ ID NO: 557 - protein, 89 aa, unidentified
  source 1..89: note = MG190-64 ribosomal protein S15
MALTQQRKQELISEYQVHETDTGSAEVQIAMLTERINRLSQHLKGNQKDFSSRRGLLKLIGQRKSLLSYVQSEDRQKYQALIARLGIRG

SEQ ID NO: 558 - protein, 89 aa, unidentified
  source 1..89: note = MG190-65 ribosomal protein S15
MTLTQERKQEIIEGYQVHETDTGSADVQVAMLTERINRLSMHLRSNKKDHASRRGLLKMIGQRKRLLSYIRNGDTAHYQALIQRLGIRG

SEQ ID NO: 559 - protein, 89 aa, unidentified
  source 1..89: note = MG190-66 ribosomal protein S15
MTLTQERKHEIIEGYQVHETDTGSADVQIAMLTERINRLSEHLKSNKKDHSSRRGLLKMIGLRKRLLSYLRKEDAQRYQALIQRLGIRG

SEQ ID NO: 560 - protein, 89 aa, unidentified
  source 1..89: note = MG190-67 ribosomal protein S15
MSLTQERKHEIIEGYQVHETDTGSAEVQIAILTERINRLSEHLKANSKDHASRRGLLQLIGRRKRLLSYMRRENAERYPALIQRLGIRG

SEQ ID NO: 561 - protein, 106 aa, unidentified
  source 1..106: note = MG190-68 ribosomal protein S15
MRVITWETAGLYESNLSMTLTQERKHELIDGYQVHDTDTGSVDVQVAMLTERINRLSNHLKTNKKDHSSRRGLLQMIGRRKRLLSYLRKEDIARYQNLIQRLGIRG

SEQ ID NO: 562 - protein, 89 aa, unidentified
  source 1..89: note = MG190-69 ribosomal protein S15
MALTQQRKQEIMGDFQTHETDTGSADVQVAMLTDRITKLSAHLKINQKDFASRRGLMMMISRRKRLLAYIQKQNVDRYKALIARLGIRG

SEQ ID NO: 563 - protein, 89 aa, unidentified
  source 1..89: note = MG190-70 ribosomal protein S15
MTLTQERKQEILQGYQVHETDTGSADVQIAMLTERINRLSEHLKTNKKDHSSRRGLLKMIGQRKRLLGYLRKENIERYQGLIQRLGIRG

SEQ ID NO: 564 - protein, 89 aa, unidentified
  source 1..89: note = MG190-71 ribosomal protein S15
MSLTQERKHELINGYQVHETDTGSADVQIAMLTERINRLSEHLKSNKKDHSSRRGLLQMIGRRKRLLTYLRNENLGRYQALIQRLGIRG

SEQ ID NO: 565 - protein, 89 aa, unidentified
  source 1..89: note = MG190-72 ribosomal protein S15
MTLPQQRKHEIMTEYQVHETDTGSTDLQIAMLTERINRLSTHLKANKKDHASRRGLLTMIGTRKRLLSYLQQEDRPRYQALIGRLGIRG

SEQ ID NO: 566 - protein, 89 aa, unidentified
  source 1..89: note = MG190-73 ribosomal protein S15
MALTQQRKQELITGYQVHETDTGSSEVQIAMLTDRITRLSEHLRANQQDHSSRRGLLKIIGQRKRLLSYVQKQNREKYQALIARLGIRG

SEQ ID NO: 567 - protein, 89 aa, unidentified
  source 1..89: note = MG190-74 ribosomal protein S15
MPLLQQRKQELISDYQVHETDTGSADVQVAMLTERINRLSSHLKTNKKDHASRKGLLRMIGLRKRLLSYIQKQDNARYRALITRLGVRG

SEQ ID NO: 568 - protein, 89 aa, unidentified
  source 1..89: note = MG190-75 ribosomal protein S15
MSLTQQRKQELMTEYQIHETDTGSADLQIAILTERINRLSAHLKINKKDHASRRGLLQIIGHRKRLLAYIQKGNKERYQALIARLGIRG

SEQ ID NO: 569 - protein, 89 aa, unidentified
  source 1..89: note = MG190-76 ribosomal protein S15
MTLPQQRKHELMTEYQVHETDTGSTDLQIAMLTERINRLSTHLKANKKDHASRRGLLKMIGTRKRLLSYLQKEDKARYQALIGRLGIRG

SEQ ID NO: 570 - protein, 89 aa, unidentified
  source 1..89: note = MG190-77 ribosomal protein S15
MALTQQRKQEIMGDFQTHETDTGSADVQVAMLTDRITKLSAHLKINQKDFASRRGLMMMISRRKRLLAYLQRENVDRYKALIARLGIRG

SEQ ID NO: 571 - protein, 114 aa, unidentified
  source 1..114: note = MG190-78 ribosomal protein S15
MSSSKKILSQKSNIIQRHQIHDDDTGSPEVQIAILTAEIKNLTEHLKKNPKDYSSRVGLLRKVGRRARLLRYLSSVSLSRYKKTIAANNIKDKLSAGLVASNNSSDDSNNSKDE

SEQ ID NO: 572 - protein, 92 aa, unidentified
  source 1..92: note = MG190-79 ribosomal protein S15
MSLLQERKQELINDYQVHATDTGSPEVQIALLSARISQLSEHLRTHKKDFSSQRGLLRLISQRRQLLLYLRKHHFDRYEAVIQRLGIRGLRS

SEQ ID NO: 573 - protein, 89 aa, unidentified
  source 1..89: note = MG190-80 ribosomal protein S15
MPLLQERKQQVIDSYRTHPTDTGSSDVQIALLSDRVLQLTTHLKEHPKDFSSRRSLLKIIGQRKRLLAYVRRRNPAHYKELITRLGIRG

SEQ ID NO: 574 - protein, 89 aa, unidentified
  source 1..89: note = MG190-81 ribosomal protein S15
MSLTQERKHEIIEGYQIHETDTGSAHVQVAMLTERINRLSEHLKQNQKDHSSRRGLMKMIGQRKRLLSYLRNEEPDKYSALIQRLGLRG

SEQ ID NO: 575 - protein, 89 aa, unidentified
  source 1..89: note = MG190-82 ribosomal protein S15
MPLLQEKKQEILLAYRRHTTDTGSSEVQVALLTGRINQLSEHLKVHPKDFACRRSLLKLIGQRKRLLAYIRREDRQQHKDLIEKLGIRG

SEQ ID NO: 576 - protein, 89 aa, unidentified
  source 1..89: note = MG190-83 ribosomal protein S15
MALQHDLKQDIINTYQIHSTDTGSADVQVAILTERIKQLSEHLKTNKKDHASRRGLLKIIGRRKRLLAYINKHDSQRYRQLIERLGIRG

SEQ ID NO: 577 - protein, 89 aa, unidentified
  source 1..89: note = MG190-84 ribosomal protein S15
MPLLQEQKQEILSVYQRHSTDTGSSDVQVALLTGRIAQLSNHLKLHPKDFASRRSLLKLIGQRKRLLAYIRREDRNRYRELVQKLGIRG

SEQ ID NO: 578 - protein, 89 aa, unidentified
  source 1..89: note = MG190-85 ribosomal protein S15
MALLQERKHEIIADYQRHETDTGSADVQVAMLTERINRLSEHLKSNKKDHSSRHGLLKMIGLRKRLLSYIQAEDRERYKALIGRLGIRG

SEQ ID NO: 579 - protein, 89 aa, unidentified
  source 1..89: note = MG190-86 ribosomal protein S15
MALLQQRKQELISEYQIHETDTGSADLQVAMLSERINRLSLHLRSNKKDHASRMGLMKMIGTRKRLLSYIQKQDEKRYRALIAKLGIRG

SEQ ID NO: 580 - protein, 89 aa, unidentified
  source 1..89: note = MG190-87 ribosomal protein S15
MSLNQAAKHSIMENYRVHETDTGSPEVQVAILTEKINRLTQHLKLNKKDYSSQRGLLRMIGQRRRLLSYLQNIDKNRYGQLIQRLGIRG

SEQ ID NO: 581 - protein, 95 aa, unidentified
  source 1..95: note = MG190-88 ribosomal protein S15
MFSMSLLQEQKQALINEYQMHATDTGSPEVQIALLSTRINQLSEHLRTHKKDFSSQRGLLRLISQRRQLLLYLRKHHFDRYENVIKRLGIRGLRS

SEQ ID NO: 582 - protein, 89 aa, unidentified
  source 1..89: note = MG190-89 ribosomal protein S15
MALLQQRKQELISEYQIHETDTGSADVQVAMLTERINRLSEHLRGNKKDHSSRMGLLKMIGQRKRLLAYIQKQDRDRYKALIGRLGIRG

SEQ ID NO: 583 - protein, 89 aa, unidentified
  source 1..89: note = MG190-90 ribosomal protein S15
MALVQHQKQQIISDYQVHGTDTGSADVQVALLTERINRLSQHLQANKKDHTSRRGLLKMIGRRKQLLAYILRHDEQHYRGLIERLGIRG

SEQ ID NO: 584 - protein, 89 aa, unidentified
  source 1..89: note = MG190-91 ribosomal protein S15
MALTQTRKQELITEYQVHETDTGSPDLQVALLTERISQLTSHLQANPKDHASRRGLLKMIGKRRSLLGYINKQEPARYQALIQRLGIRR

SEQ ID NO: 585 - protein, 89 aa, unidentified
  source 1..89: note = MG190-92 ribosomal protein S15
MALVQQRKQEIITAYQVHETDTGSADVQVAMLTERINRLSEHLKTNKKDHSSRHGLLKMIGLRKRLLAYIQRNDRARYRALIERLGIRG

SEQ ID NO: 586 - protein, 89 aa, unidentified
  source 1..89: note = MG190-93 ribosomal protein S15
MALVQEKKQELINSYQIHETDTGSADVQVAMLTERINRLSAHLKTNKKDFSSRRGLLKMIGQRKRLLSYIIKQDQQHYRELITRLGIRG

SEQ ID NO: 587 - protein, 89 aa, unidentified
  source 1..89: note = MG190-94 ribosomal protein S15
MSLTQESKQEIIDGYQVHETDTGSADVQIAMLTARISQLSSHLKNNKKDHSSRRGLLKMIGRRKRLMSYLRKQDRERYQALIERLGIRG

SEQ ID NO: 588 - protein, 89 aa, unidentified
  source 1..89: note = MG190-95 ribosomal protein S15
MVLVQEQKQNIINEYQIHETDTGSADVQVAMLTERINRLSSHLKTNKKDFSSRRGLLRMIGRRKRLLSYILKQDQARYRELITRLGIRG

SEQ ID NO: 589 - protein, 89 aa, unidentified
  source 1..89: note = MG190-96 ribosomal protein S15
MALTQERKQEIIDSYQIHDTDTGSADVQIAMLSDRISRLSTHLQANKKDHASRRGLLKMIGQRKRLLSYVREGNPEHYQALIKRLGIRG

SEQ ID NO: 590 - protein, 89 aa, unidentified
  source 1..89: note = MG190-97 ribosomal protein S15
MSLTQEQKQEIITEHQVHETDTGSPEIQVAMLTKRINQLSAHLKQNKKDYSSTRGLLKMIGHRKRMLAYIRNKDNDKYRALIQRLGIRG

SEQ ID NO: 591 - protein, 89 aa, unidentified
  source 1..89: note = MG190-98 ribosomal protein S15
MSITQERKQELISEYQVHGTDTGSSDVQVAILSDRINSLTQHLKVNKKDHASRLGLLKLIGRRRRLLTYIQKQDYEHYQQLIRRLGIRR

SEQ ID NO: 592 - protein, 89 aa, unidentified
  source 1..89: note = MG190-99 ribosomal protein S15
MALLQERKQELISEYQVHETDTGSADVQVAMMTERIDKLSQHLHSNKKDYSSRRGLLKMIGRRKRLLSYIAKKDVNQYRELIGRLGIRR

SEQ ID NO: 593 - protein, 89 aa, unidentified
  source 1..89: note = MG190-100 ribosomal protein S15
MALLQEQKQQIISEYQVHETDTGSADVQVAMLTERINQLSAHLKTNKKDHSSRRGLLKIIGQRKRLLSYILKQDQERYRALIKRLGIRG

SEQ ID NO: 594 - protein, 89 aa, unidentified
  source 1..89: note = MG190-101 ribosomal protein S15
MALLQERKQELISEYQVHETDTGSAEVQVAMLTERINKLSQHLRDNKKDYSSRRGLLKMIGRRKRLLSYIAKKDVERYRELIGRLGIRR

SEQ ID NO: 595 - protein, 92 aa, unidentified
  source 1..92: note = MG190-102 ribosomal protein S15
MSLIQEQKQALINEYQIHATDTGSPEVQIALLSARINRLSEHLRTHKKDFSSQRGLLRLISQRKQLLLYLRKHHPDRYEALIQRLGIRGLRA

SEQ ID NO: 596 - protein, 91 aa, unidentified
  source 1..91: note = MG190-103 ribosomal protein S15
MALLQQEKQQIIESYRLHDTDTGSAEVQVALLTSRINQLSQHLQRNPKDFNSRRGLLMMIGRRKRLLNYIAKHSPDRFRELAERLNIRVKK

SEQ ID NO: 597 - protein, 91 aa, unidentified
  source 1..91: note = MG190-104 ribosomal protein S15
MALLQQEKQEIIETYRLHDTDTGSAEVQVALLTSRINQLSQHLQKNPKDFNSRRGLMMMIGRRKRLLNYIAKRSPDRFRELAERLNIRVKK

SEQ ID NO: 598 - protein, 91 aa, unidentified
  source 1..91: note = MG190-105 ribosomal protein S15
MALLQKEKQEIIERYRLHDTDTGSADVQVALLTSRINQLSQHLQRNPKDFNSRRGLLMMIGRRKRLLNYIAKHHPERFRELVERLNIRVKK

SEQ ID NO: 599 - protein, 113 aa, unidentified
  source 1..113: note = MG190-106 ribosomal protein S15
MSSSKKILSQKSNIIQQHQIHDDDTGSPEVQIAILTAEIKNLTEHLKKNPKDYSSRVGLLRKVGRRARLLRYLSSVSLSRYKKTIAANNIKDKLSAGLVASDNSSDDSNSKDE

SEQ ID NO: 600 - protein, 114 aa, unidentified
  source 1..114: note = MG190-107 ribosomal protein S15
MSSSKKILSQKSNIIQQHQIHDDDTGSPEVQIAILTAEIKNLTEHLKKNPKDYSSRVGLLRKVGRRARLLRYLSSVSLSRYKKTIAANNIKDKLSAGLVASDNSSDDSNNNKDE

SEQ ID NO: 601 - protein, 89 aa, unidentified
  source 1..89: note = MG190-108 ribosomal protein S15
MALLQERKQEIISDYQIHETDTGSADVQVAILTERINRLSAHLKENKKDHASRRGLLKMIGQRKRLLAYILKHNPDRYRALINRLGIRG

SEQ ID NO: 602 - protein, 89 aa, unidentified
  source 1..89: note = MG190-109 ribosomal protein S15
MALLQERKQEIISDYQVHETDTGSADVQVAILTERINRLSAHLRENKKDHASRRGLLKMIGQRKRLLAYILKQDQERYRALIGRLGIRG

SEQ ID NO: 603 - protein, 89 aa, unidentified
  source 1..89: note = MG190-110 ribosomal protein S15
MALTQQRKQEIISQYQVHETDTGSADVQIAMLTERINRLSEHLQVNKKDFSSRRGLLKLIGQRKRLLSYIQKENREHYQALISRLGIRG

SEQ ID NO: 604 - protein, 89 aa, unidentified
  source 1..89: note = MG190-111 ribosomal protein S15
MPLLQQRKQEIISEYQVHETDTGSAEVQVAMLTERINRLSTHLRSNKKDHASRMGLMKMIGARKRLLGYIQKKDEQHYRDLIGKLGIRG

SEQ ID NO: 605 - protein, 89 aa, unidentified
  source 1..89: note = MG190-112 ribosomal protein S15
MALLQERKQEIISDYQIHETDTGSADVQVAMLTARINRLSEHLKSNKKDHSSRMGLLKMIGHRKRLLAYIQKQDNDRYRALITKLGIRG

SEQ ID NO: 606 - protein, 89 aa, unidentified
  source 1..89: note = MG190-113 ribosomal protein S15
MALTQQRKQEIISQYQVHETDTGSADVQIAMLTERINRLSEHLQANKKDFSSRRGLLKLIGQRKRLLSYIQKENREHYQALISRLGIRG

SEQ ID NO: 607 - protein, 89 aa, unidentified
  source 1..89: note = MG190-114 ribosomal protein S15
MPLAQERKQELISGYQVHETDTGSPEVQVAILSDRINQLTEHLRAHPKDFSSRRGLLKLIGRRRQLLSYLQKNENDRYRALVERLGLRR

SEQ ID NO: 608 - protein, 89 aa, unidentified
  source 1..89: note = MG190-115 ribosomal protein S15
MALTQQRKQEIISSYQVHETDTGSADVQIAMLTARINRLSEHLQANKKDHSSRRGLLKLIGQRKRLLAYIQQDSREKYQALIGRLGIRG

SEQ ID NO: 609 - protein, 89 aa, unidentified
  source 1..89: note = MG190-116 ribosomal protein S15
MGLPQQRKQELMLEYQIHETDTGSAEVQVAMLTARINQLSSHLENNSKDHAGRRGLLKMIGQRKRLLSYILKQDRGRYQALIGRLGIRG

SEQ ID NO: 610 - protein, 89 aa, unidentified
  source 1..89: note = MG190-117 ribosomal protein S15
MALTQAEKQAIMADYQVHETDTGSADLQVAMLTKRINQLTQHLKANKKDHSSRRGLLRMIGRRKRLLAFIEKEDRSRYLELIGRLGIRR

SEQ ID NO: 611 - protein, 89 aa, unidentified
  source 1..89: note = MG190-118 ribosomal protein S15
MPLSQARKQELMTEYQIHETDTGSADFQVAVLTERISQLSQHLQKNKKDFASQRGLMQMIGRRKRLLGYIRKQDEERYRHLIRRLGIRG

SEQ ID NO: 612 - protein, 89 aa, unidentified
  source 1..89: note = MG190-119 ribosomal protein S15
MALLQEQKQQLISDYQIHETDTGSADVQVAMLTERINRLSAHLKENKKDHASRRGLLKMIGQRKRLLAYILKHDQDRYRALIGKLGIRG

SEQ ID NO: 613 - protein, 89 aa, unidentified
  source 1..89: note = MG190-120 ribosomal protein S15
MPLLQARKQELISEYQVHETDTGSAVVQVAMLTERINKLSSHLQSNQKDYSSRRGLLKMIGRRKRLLSYIAKHNVDEYRELIGRLGLRR

SEQ ID NO: 614 - protein, 89 aa, unidentified
  source 1..89: note = MG190-121 ribosomal protein S15
MALLQERKQQIISDYQIHETDTGSADVQVAMLTERINRLSAHLKENKKDHASRRGLLKMIGQRKRLLAYIQKHDQDRYRALIGKLGIRG

SEQ ID NO: 615 - protein, 89 aa, unidentified
  source 1..89: note = MG190-122 ribosomal protein S15
MPLLQEQKQEILTTYQKHSTDTGSSDVQVALLTGRITQLSNHLKLHPKDFASRRSLLKLIGQRKRLLAYIRREDRNRYRELVQKLGIRG

SEQ ID NO: 616 - protein, 89 aa, unidentified
  source 1..89: note = MG190-123 ribosomal protein S15
MPLLQERKQEVINSFRIHPTDTGSSDVQIALLSDRVVQLTNHLKEHPKDFSSRRSLLKIIGQRKRLLAYVRRRDPAHYQELITRLGIRG

SEQ ID NO: 617 - protein, 92 aa, unidentified
  source 1..92: note = MG190-124 ribosomal protein S15
MSLLQERKQELINEYQMHATDTGSPEVQIALLTDRINQLSEHLRTHKKDFSSQRGLLRLISQRRQLLLYLRKHHLDRYETLIKRLGIRGLRS

SEQ ID NO: 618 - protein, 92 aa, unidentified
  source 1..92: note = MG190-125 ribosomal protein S15
MSLLQEQKQALINEYQMHATDTGSPEVQIALLTDRINQLSEHLRTHKKDFSSQRGLLRLISQRRQLLLYLRKHHLDRYETLIKRLGIRGLRS

SEQ ID NO: 619 - protein, 89 aa, unidentified
  source 1..89: note = MG190-126 ribosomal protein S15
MPLLQEQKQEILSVYQRHSTDTGSSDVQVALLTGRIAQLSNHLKLHPKDFASRRSLLKLIGQRKRLLAYIRREDRNRHRELVQKLGIRG

SEQ ID NO: 620 - protein, 89 aa, unidentified
  source 1..89: note = MG190-127 ribosomal protein S15
MPLLQEQKKEILSLYQRHSTDTGSPEVQIALLTGRINQLSNHLKLHPKDFDSRRSLLKLIGQRKRLLAYLRREDRSRYQELVEKLGIRG

SEQ ID NO: 621 - protein, 104 aa, unidentified
  source 1..104: note = MG190-128 ribosomal protein S15
MLSSMGSKHFQSAMPLPTARKQEIMAARQIHPTDTGSPDVQIALLTERINQLSGHLQNNPKDYNSRRGLLMMIGKRKRLLSYLAKIDEERYRRLVEELNIRVRK

SEQ ID NO: 622 - protein, 92 aa, unidentified
  source 1..92: note = MG190-129 ribosomal protein S15
MSLIQEQKQALINEYQIHATDTGSPEVQIALLSARINRLSEHLRTHKKDFSSQRGLLRLISQRRQLLLYLRKHHPDRYEALIQRLGIRGLRA

SEQ ID NO: 623 - protein, 89 aa, unidentified
  source 1..89: note = MG190-130 ribosomal protein S15
MTLLQARKQELISDYQVHDTDTGSADVQIAMLTDRINQLSAHLQKNKKDYSSRRGLLKMIGHRKRLMAYLLKQDSERYRALIQKLGIRG

SEQ ID NO: 624 - protein, 89 aa, unidentified
  source 1..89: note = MG190-131 ribosomal protein S15
MALLQERKQELISEYQVHETDTGSAEVQVAMLTERINKLSQHLQSNKKDYSSRRGLLKMIGRRKRLLSYIANKDAGKYRELIGRLGIRR

SEQ ID NO: 625 - protein, 89 aa, unidentified
  source 1..89: note = MG190-132 ribosomal protein S15
MALTQQKKQEIMTEHQTHETDTGSAEVQVALLSERITSLSAHLKVHKKDYSSTRGLLQIIGRRKRLLSFIRQKNPSGYQDLIKRLGIRG

SEQ ID NO: 626 - protein, 89 aa, unidentified
  source 1..89: note = MG190-133 ribosomal protein S15
MSLTQEQKQQIITEHQVHETDTGSPEVQVAMLTERINQLSAHLKKNKKDYSSTRGLLKMIGHRKRLLAYIRNKDNDKYRALIQRLGIRG

SEQ ID NO: 627 - protein, 89 aa, unidentified
  source 1..89: note = MG190-134 ribosomal protein S15
MSLTQERKHEIIDGYQLHETDTGSAEVQVAMLSERISRLTEHLKVNSKDHASRRGLLQIIGRRKRLLAYIRKGDKQRYLNLIQRLGIRG

SEQ ID NO: 628 - protein, 89 aa, unidentified
  source 1..89: note = MG190-135 ribosomal protein S15
MSLTQEKKQELITQYQVHETDTGSSEVQVAMLTERINRLSKHLQANKKDHSSRRGLLKMIGQRKRLLSYIQSGDRERYKTLIRSLGIRG

SEQ ID NO: 629 - protein, 107 aa, unidentified
  source 1..107: note = MG190-136 ribosomal protein S15
MAVIILETAALIKEKSRVMSLTQERKQELISQYQVHETDTGSSDVQVAMLTDRINRLSKHLQVNKKDHSSRRGLLKMIGQRKRLLSYIQKGDRERYKTLIRSLGIRG

SEQ ID NO: 630 - protein, 89 aa, unidentified
  source 1..89: note = MG190-137 ribosomal protein S15
MALTQQRKQEIMSEHQTHETDTGSCEVQVAMLTERISKLSEHLKINKKDHASRRGLLQMISRRKSLLGFLQRLDKSRYQALIARLGIRG

SEQ ID NO: 631 - protein, 89 aa, unidentified
  source 1..89: note = MG190-138 ribosomal protein S15
MALTQQRKQEIMGEYQAHETDTGSADLQVAMLSDRINQLSLHLRANQNDFSSRRGLMQLIGRRRRLLSYIKKQNKERYQALIARLGIRG

SEQ ID NO: 632 - protein, 89 aa, unidentified
  source 1..89: note = MG190-139 ribosomal protein S15
MALTQQRKLEIMGEYQTHETDTGSADLQVAMLTDRISKLSAHLKINQKDFASRRGLMLMISRRKRLLSYIQKQSVDRYKALIARLGIRG

SEQ ID NO: 633 - protein, 89 aa, unidentified
  source 1..89: note = MG190-140 ribosomal protein S15
MALTQQEKQELMSEYQIHETDTGSADLQVAMLTKRISQLTEHLKINKKDHSSRLGLLKMIGRRKRLLAYIQKGDPQRYQSLIARLGIRR

SEQ ID NO: 634 - protein, 89 aa, unidentified
  source 1..89: note = MG190-141 ribosomal protein S15
MSLTQEKKQELISQYQVHETDTGSAQVQVAMLTERINRLSKHLQANKKDHSSRRGLLKMIGQRKRLLSYIQKGDRDRYKTLIRSLGIRG

SEQ ID NO: 635 - protein, 89 aa, unidentified
  source 1..89: note = MG190-142 ribosomal protein S15
MALTQQQKQELMTEYQVHETDTGSADLQVAMLTKRIEQLTQHLKVNKKDHSSRKGLLKMIGRRKRLLAYIQKGDPQRYQTLIGRLGIRR

SEQ ID NO: 636 - protein, 89 aa, unidentified
  source 1..89: note = MG190-154 ribosomal protein S15
MTLLQERKQELIAEYQIHETDTGSVDLQIAMLTERINQLSAHLQKNKKDYSSRRGLLKMIGQRKRLMAYLLKKDTERYRNLIQKLGIRG

SEQ ID NO: 637 - protein, 89 aa, unidentified
  source 1..89: note = MG190-155 ribosomal protein S15
MTLLQERKQELISEYQVHETDTGSAEVQVAMLTERINKLSQHLQSNKKDYSSRRGLLKMIGRRKRLLSYIAKKDVNQYRELIGRLGIRR

SEQ ID NO: 638 - protein, 89 aa, unidentified
  source 1..89: note = MG190-156 ribosomal protein S15
MSLLQEQKHQIISDYQVHETDTGSADVQVAMLTERINRLSDHLKANKQDHSSRRGLLQMIGRRKRLLAYIRKQDLERYQALIKRLGIRG

SEQ ID NO: 639 - protein, 114 aa, unidentified
  source 1..114: note = MG190-157 ribosomal protein S15
MSSKHIQAAKPVIVSKHQIHKTDTGSPEVQVAILTEEITKLTDHLKINPKDHSSRRGLLRKVSRRKKLLNYLLGEDKVRYIRTCKKNGIRTNAAVMLTMNHPKKVALAEDEKAE

SEQ ID NO: 640 - protein, 89 aa, unidentified
  source 1..89: note = MG190-158 ribosomal protein S15
MTLLQARKQELISDFQVHETDTGSADLQIAMLTARISQLSEHLQKNKKDYSSRRGLLKMIGQRKRLMGYLQKQDSERYRALIQKLGIRG

SEQ ID NO: 641 - protein, 89 aa, unidentified
  source 1..89: note = MG190-159 ribosomal protein S15
MALTQQRKQELISDYQVHETDTGSSEVQIAMLTERINRLSEHLRANQQDHSSRRGLLKLIGQRKQLLAYVQKSNKEKYQALIARLGIRG

SEQ ID NO: 642 - protein, 89 aa, unidentified
  source 1..89: note = MG190-160 ribosomal protein S15
MALTQERKQELISSYQVHETDTGSAAVQIAMLTERINRLSEHLKSNKKDHSSRRGLLKIIGQRKRLLSYLQTEDREQYQNLIGRLGIRG

SEQ ID NO: 643 - protein, 89 aa, unidentified
  source 1..89: note = MG190-161 ribosomal protein S15
MSLTQERKHEIIEGYQVHETDTGSAEVQIAILTERINRLSEHLKANSKDHSSRRGLLQLIGRRKRLLAYMRRESAERYPALIQRLGIRG

SEQ ID NO: 644 - protein, 89 aa, unidentified
  source 1..89: note = MG190-162 ribosomal protein S15
MALTQQRKQEIINNFQVHGTDTGSTDVQIAMLTERINRLSEHLQANKKDHSSRRGLLKLIGHRKRLLAYLQQESREKYQALISRLGIRG

SEQ ID NO: 645 - protein, 105 aa, unidentified
  source 1..105: note = MG190-163 ribosomal protein S15
MGDCYIHLEKLETESIMALTQLRKQEIISNYQVHETDTGSADVQVAMLTERINRLSEHLQANKKDHSSRRGLLKLIGQRKRLLAYISQESREKYQALIARLGIRG

SEQ ID NO: 646 - protein, 89 aa, unidentified
  source 1..89: note = MG190-164 ribosomal protein S15
MALTQQRKQELISGFQVHETDTGSADVQIAMLTDRINRLSQHLQANKKDHSSRRGLLKMIGQRKRLLAYIQQNNREKYQALIARLGIRG

SEQ ID NO: 647 - protein, 89 aa, unidentified
  source 1..89: note = MG190-165 ribosomal protein S15
MALNQQRKQEVMTSYQVHETDTGSADVQVALLTERINKLSEHLKANSKDHSSRRGLLKMISLRKRLLAYILKQDQQRYRKLIERLGIRG

SEQ ID NO: 648 - protein, 89 aa, unidentified
  source 1..89: note = MG190-166 ribosomal protein S15
MALVQERKQEIITEFQVHETDTGSADVQVAMLTERINKLSLHLRSNKKDHASRTGLLKMIGQRKRLLAYIQKGDKDRYRALITRLGIRG

SEQ ID NO: 649 - protein, 89 aa, unidentified
  source 1..89: note = MG190-167 ribosomal protein S15
MALLLERKQELLSSYQTHPTDTGSSQVQVAMLTERVNQLSSHLKTHPKDFSSRRSLLKMIGQRKRLLAYIKQGSQTDYKELIQRLGVRG

SEQ ID NO: 650 - protein, 92 aa, unidentified
  source 1..92: note = MG190-168 ribosomal protein S15
MTLTQERKQEIMSQYQLHATDTGSSALQVAMLTERINRLSEHLKTNKSDHASRRGLLKMIGQRKRLLAFVQAESVQSYQNLADSLGIRRVKD

SEQ ID NO: 651 - protein, 89 aa, unidentified
  source 1..89: note = MG190-169 ribosomal protein S15
MALLQQRKQEIITDYQIHETDTGSSEVQVAMLTDRINKLSLHLRTNKKDHASRMGLLKMIGQRKRLLAYINKGSQERYRALITRLGIRG

SEQ ID NO: 652 - protein, 89 aa, unidentified
  source 1..89: note = MG190-170 ribosomal protein S15
MALLQERKLEIFAEYQKHPTDTGSSDVQVAMLTERVTQLTVHLKLHPKDFSSRRSLLKIIGQRKRLLAYVRNEDRAHYKQLIQSLGVRG

SEQ ID NO: 653 - protein, 89 aa, unidentified
  source 1..89: note = MG190-171 ribosomal protein S15
MPLLQQRKQELISDYQVHETDTGSSDVQVAMLTERINRLSEHLKTNKKDHASRKGLLGMIGLRKRLLSYIQKQDNARYRALITRLGIRG

SEQ ID NO: 654 - protein, 89 aa, unidentified
  source 1..89: note = MG190-172 ribosomal protein S15
MALLQERKLEIFSEYQKHPTDTGSSDVQVALLTERVTQLTAHLKLHPKDFFFFLSLLKIIGQRKRLLAYVRNQDRAHYKQLIQSLGVRG

SEQ ID NO: 655 - protein, 89 aa, unidentified
  source 1..89: note = MG190-173 ribosomal protein S15
MALLQQRKHEIIADYQVHEMDTGSSDVQVAMLTERINRLSEHLKVNKKDHASRKGLLGMIGLRKRLLAYIQAQDKARYRALITRLGIRG

SEQ ID NO: 656 - protein, 89 aa, unidentified
  source 1..89: note = MG190-174 ribosomal protein S15
MALLQERKLEIFSEYQKHPTDTGSSDVQVALLTERVTQLTTHLKLHPKDFSSRRSLLKIIGQRKRLLAYVRNQDRAHYKQLIQSLGVRG

SEQ ID NO: 657 - protein, 89 aa, unidentified
  source 1..89: note = MG190-175 ribosomal protein S15
MSLVQEDKQKIITDFQKHETDTGSVEVQVAMLTERINRLSGHLKTNKKDHGSRIGLLKMISLRKRLLSYVQKLDYARYKTLIGRLGIRG

SEQ ID NO: 658 - protein, 89 aa, unidentified
  source 1..89: note = MG190-176 ribosomal protein S15
MALTQEKKQELIEGFKTHSTDTGSPEVQVAMLTERITQLTQHLRVNPKDFASRRGLLKIISQRKQLLGYVAKMDTPRYQKIVERLGLRR

SEQ ID NO: 659 - protein, 89 aa, unidentified
  source 1..89: note = MG190-177 ribosomal protein S15
MALTQQRKQELICNFQVHETDTGSADVQIAMLTERINRLSEHLQANKKDHSSRRGLLKMIGQRKRLLSYIHGENREKYQALIGRLGIRG

SEQ ID NO: 660 - protein, 808 aa, unidentified
  source 1..808: note = MG64-98 effector
MVMKTTRFDVIARKDPKYKKNREKPIDQLEEEALWEVVQASCHHTPLGIEILKQMEQPSAFPARIEKLKQPQADGILPDIEQEKKWLEAEIKKVCDSLKQQVAFQSLPGRIYSSAVHQSLKPLKGWLEKQWQLLLSISGKKRFLAVVETDADLAQASDFSWSDIRASAQAILQQTQEEIAAKAEDETAAKDTKQLLNALLKQYEATSDILTCRAIIHLLRNNFKVRRKPENPEKVQEWLEGKRVEIERLEEQVPRLPRLRNLFPDQAYDEGLEGLITYPLSGVAVSERIEWLFHYRVLICFFLIYITSVEKSIQLAYCLLHLVRAEVEREEVQFYEWHDDVSDKIDQFLTIPKSLPYPIYFGGDDLRGWQLNQEGRICFKLNGLGDYLFEVRCDRRQLGIVKYFLQDWQTRNKSKKEYSGGLTLLRSAELLVKPKSGKQNAKLPPVDDRQAVVAGYKLSLHCTYDTDYLSRQGLERVRQRKIAGQLKNLTDKQAKLTKQQAKLQQLEQEMQQEQAGTSPRRRSKRNAQRLEQIEKLKQSISDLQAELERLRPKLERLQQSQLFQRADRPLYEGVANLFVGVCLDLDQHLVVTVVDAMRRKVLTKRTVKQIMGKHYSLLQRYRHLKQEHDKQRQQDQKVGRHNHLSETDLGKQVADAIANGLIALAQQYKVSTIVLPETKGWRERLYSQLAARAKIKCNGSKKAMARYTKQYGKRLHQWDYNRLSQAIETEAQTVGLTVIFQRPEFRQEAHEQGNQSADEANEQDNQRVNPFELALQIAIAAYDSLQAEDNAEESESADGTLPDSAGDT

SEQ ID NO: 661 - protein, 630 aa, unidentified
  source 1..630: note = MG64-99 effector
MSQITIQCRLIAPETTRRYLWELASEKNTPLINELIQGVVSHPDFEIWRQKGRHPTDVVCKHCSQLKAEARFSGQPSRFYMSAEKVVNYIFKSWFKIQNRLQQRLSGKQKWLNILKSDEELAEICGQPLEKIKKKAIQILKDTKQKYEASQTEEAQSSSQKSFIRSRLFKRYRSAKQPLTQCAIAYLLKNNCKIPKQPEDPQKFSQRRRKSELQAKRLQEQLEARIPKGRDLTGQAWLSTLLTAASAVPKDNQEYRRWQDRLLTKPRTIPFPILFETNEDLSWSLNPEGRLCVHFNGLREHTLQIYCDQRQLPWFKRFLEDQQTKRANKNKHSSALFTLRSARISWQEIDTKGHPWDNHYLTLSCTVDKRLWSAEGTDEVRQEKAADTAKILTRLNEKDSLSKTQAAYTRRLASTLERLESSFDRPSQPRYQGQSQIIVGLSLGWDSPLTLAIWKADIQEIVVYRSLQQLLDKDYPLFLKQRREQQKQSHQRHKAQRHGKGNQFGTSNLGQHIDRLLAKAVVKTAKQYGAGSIAIPALDNIRDILQTEIDARAEQKIPGYLEVQKRYTKQYKSNIHKWSYGRLLDQIISKANQESLAIEKSKQPLSGTPQAKAKAVAINAYELRKTVHQN

SEQ ID NO: 662 - protein, 712 aa, unidentified
  source 1..712: note = MG64-100 effector
MSLITIQCRLVADKASLRHLWRLMAEKNTPLINQLLEQLGQHPNFETWLQKGEVPEDTIKTICNSLKTQERFADQPGRFYTSAVTLVKEAYKSWFALQQQQQRQIKGKERWLKMLKSDIELQQESQCNLNVIRAKATEILDSFFAKFTQDKNKQSKTKKANNTKKNKKISSNTTLFGRLFDTYDKTEDCLSKCALVYLLKNNCQVSKVDEDPEQYAKNKRKKEIEIERLRNKIKSQNPKGRDITAEKWLGTLEEATKKVPLNEDEAKSWQASLLKRYNYMPYPIDYESSTDLEWFTNSVDNEKHTGLHNLKNFDSKTKIAIVVFWQIYFLNLALKLKIYSLMKYVYFTMGYYPNKDVNWLNLKNKEGCIFVKFNGLKEKIRNPEFYVCCDSRQLHYFQRFCQDWQILHQDKETYSCGLFILRSARLLWQERKGKGKPWTIHRLILQCSIETRLWTKEETELVRCEKINKAEKTISKMEQQGNLKKTQVNRLQKELTTRQKLNNPFPGRPSQLLYQGKSNILVGVSFGLDKPATVAVLDATSKKVLTYRSVKQLLGDNYNLLNRQRQQQQRLSHERHKAQKQNAPDSGGESELGQYIDRLLADAIVAIAKTYSAGSIVLPKLRDMREIIQSEVQAKAEKKIPGYKEGQQNYAKDYRVSVHRWSYGRLIESIQTQAAKTGISVESGSQPNIGSQQQQARDLALFAYQERQIEVL

SEQ ID NO: 663 - protein, 745 aa, unidentified
  source 1..745: note = MG64-101 effector
MSQITIQCRLVASESSRHQLWKFMADLNTPLINELLHQVNQHPEFETWRQKGKHPNSVVKELCQPLRTDPRFIDQPGRFYDSAIATVNYIYKSWLALMKRLQFQLEGKIRWLGMLKSDAELVEASGVTLESLRAKATEILAQFTLQPDTAEPQSGKEKKRKRTKKSKKLDGESSISDTFQSNTVEEPQPRKEKKRKKTKKSDGERSISDTLFEVYRGTEDNLTRCAISYLLKNGCKISQKDENAEEFANRRRKLEIQIERLIEQLEARTPKGRDLNDAKWLESLLLATHNVPENEAKAKLWQDSLLKKSSKLPFPIGYGSNGDMTWFWQFSLYNVPINLRFFWLWIYIDYLIAILFLRDALKNEKEWLHNLRINNISLLIKLWDLTVDVNCLASILFLHESFHNKYKRRICVKFNGLGEHTFKVYCDFRDLHWFYRFLEEQTIKKHYKNKYTTSLFALRTGRLCWQENEGKGKAWNVNRLILYCCVDTRLWTLEGTNEFKKEKAEEIAKSITKTKAKGELNEKQLDSIQRGNTTLANIDNPFPRPSKPLYKGQSHILVGVSFGLENPATVAVVNGSIGKVITYRNIKQLLGNNYRLLNRQRQQKHTLSHKRQIAQKIAAPNQVGESELGQYVDRLIAQEIVAIAQKYKAGSIVLPKLGDMREQIQSEVQTKAEQKSDLIEVQKKYAKQYRVSVHQWSYGRLIANIQSQAAKAGILIEESKQPIRGSPQEKAKELAIAAYHSREIN

SEQ ID NO: 664 - protein, 670 aa, unidentified
  source 1..670: note = MG64-102 effector
MPRPPAVPTQVWINPPVTPSPSHRGNTNFSLYSVSFLLTYSAVRRHLWHLMSEKNTPLVNALLKQVSQQSKFETWQRESTIPRSVIGELCEPLKKIYPDQPQRFYASAILMVTYTYESWLALQQSRRRRLDGKQRWLNVVMSDADLLALSGATLETIQQKAQTILSQLNAEPETQSNSNEKRSKQARRQTNSSNNSSLFARLFEAYEATDDTPTRCAIAYLIKNGGKIPETEEDPEKFARRIHRKQKEVEQLEAQLQARLPKGRDLTGAEFLETLASATQQLPENVVQAREWQAKLLTRPASLPYPIVFGSSTDVRWGKTAKGRISVSFNGVDKYLKEADSSIQEWFKASKEYPFRLYCDQRQLPFFQRFLQDWQDYQANKDTYPAGLLTLSSAMLAWREGEGKGEPWNVNHLTLYCSFDTRLMTAEGTLAVQQEKATKAQKRLTKPDSDPRIRSTLDRLQNLPKRPSQKPYQSNPEILVGLSIGLASPITAAVINGRTGEVLTYRTLRSLLGDHYQLLNRHRHQQQQNAVQRYRNQKRGVAFQPRESELGLYVDRLLAKAIIQLAQAYQVGSIIIPNLTHLRELLESEITAKAEQKCLGSVEAQNQYAKEYRQRIHRWSYNRLIEAIRSQANQLGITVESGFQPIRGNSQEQARDIAIAAYHSRAIAKN

SEQ ID NO: 665 - protein, 679 aa, unidentified
  source 1..679: note = MG64-103 effector
MSQITIQCRLVASESTRQQLWKLMAGVNSPLINELLEQVGQHPDFGTWRQKGKLPTGIVEKLCKPLKTDPRFIGQPARLYKSAIDIVEYIYESRLAQQQRLQHQLEGQTRWLGMLKSDSELVCRLGCSLDTIRTRAAEVLAQATLTNFVKREGEPLLHKSGVESHHSLSKEGSPSLKKTSVTCDPSNSHPSQDKKRKKTKKRNSKNHNRSLSNALFEAYLATEDVLSKCAITYLLKNGCKVSDQEEDIEKFAKRRRKVEIRVERLTEQLASRMPKGRDLKTAKWLETLFVATTTVPQNEAEARTWQASLLKKPKSVPFPIRIKTSDELLWSKNHKGRLCVRFSGFSEHTFEVYCDRRQLHWFQRFLEDQQVKRDSKNQHSSSLFTLRSGQIAWQEDEGEGEPWNVHCLTLYCIVDTRLWTEEGTEQVRQEKAADIAKVITRMKGKSDLSKTQVGFVKRKHSTLARINNPFPRPSKPLCQGHSHILVGVSLGLEKLATVAVVDASTGKALTYRSIRQLLGDNYKLLNRASSIQQHNSHERHKAQKRSAPNSFGESELGQYVDRLLSGAIIAIAQTHQAGSIVVPKLGEMREIVQSEIQARAEAKCPGCIEAQARYAKQYRCNVHSWSYGRLIQNIQSQAATAGIAVEEEPQPIRGSPQEKARELAITAYXXXXLTTTLEP

SEQ ID NO: 666 - protein, 681 aa, unidentified
  source 1..681: note = MG64-104 effector
ALMALWKSLKKEPRFKGLPNCFLKSARLMVHYTYDSWLALQKEKQTKLDKIIRWVEVVKSDTELVQISGCSLDTIRAKAQDLLVQINTQNQTQQTKTRRVRKRKSSNSSTANQAKSQFIPEAKQNFNGSAEENNKSTTNQVPEIKYKTFDTLFNTYYMSSDILQKCAIAYLIKNKLKVNNKEEDLKKFTKIVRQKKKKIERLQEQLKSRLPKGRHWIGNEFLDNLEAFGIPESETEWFSLQSALLREHNFLPYPILFGSSDDLSWSKQLKISNNSNLIKENSESEKIRERICVQFKGLKEVIFEISCDRRQLPLFQQFLKDWTIYSQNTKEHTSSLFLIRSATLIWEDTKKMKNRQKRQNENKIVNQKSIDSQQEELKKIFQKEIDGEEQPWNRYQLFLHCTVASEFLSKEGTQQLGQKKQELALKAIATLEQKILELEKEGKSTKNDRESFSRKQGTVRRLNNLDNPFKRPSRPLYQAQPNILLGVSLGSSKLATATVVDVTTEKVLECQGVRCLLGDNYKLLTRKRYLHEMHSHLRSKAQKRGAKNLLREAELGEHIDRLIATAIIALARKYQASTIVLPNMKDYTEKKQSEIEAFAEQECSGLKFVEKRFTKAQSVKLHQWSYGRLSEIICQQASKVGIAVEIGQQPRHGSSQEQGRAMAIETYHSRKNSLKSKNLRS

SEQ ID NO: 667 - protein, 640 aa, unidentified
  source 1..640: note = MG64-105 effector
MSVITIQCRLVADDKTLRHLWELMAEKNTPLVNELLDRLGKHTDFEAWVQAGKVPKTTIKALCDSLKTQEPFIGQPGRFYTSATTLVAYIYKSWLALHKRRQRKIEGKERWLEMLKSDVELEQESNSSLELIRTIATEILSKFSASSTDGRNQKSKGKKSKKVKKDKADEPMSIKPGVLFEAYQKTEDILRRSALVYLIKNNCQVNLAEEDPDKYAKMRRKKEIEIERLKEQLKSRVPKGRDLTGKKWLETLEKAVNSIPQDENEAKSWQAGLLRKSSTVPFPVAYETNEDMHWEISDKGRIFVSFNGLSKLKLEVYCDQRHLPWFQRFVEDQETKRKGKNQHSSGLFTLRSGRLSWLKQEGKAEPWSVNRLILFCSVDTRMWTVEGTQQVAIEKIADVEQNLTKAKEKGELNSNQQAFVTRQQSTLAKINTPFPRPSKPLYEGKSHILVGVSLGLENPATVAVFDAVNNKVLAYRSVKQLLGNNYNLLNRQQQQKQRLSHDRHKAQKDFTRNDFGESELGQHIDRLLAKEIVAIAVTYFAGSIVLPKLGDMREIIQSEVQARAEKKIPGFKEGQQKYAKEYRKQVHNWSYGRLIENIQSQAAKVGILIETGQQPIRGSPQEQARDLALFAYQCRIASSI

SEQ ID NO: 668 - protein, 640 aa, unidentified
  source 1..640: note = MG64-106 effector
MSVITIQCRLVAPEETLQHLWELMEKKNTPLINELLEQLGKHPDFETWLQKGKLPTEVVKTLCNSLKTNLCFAGQPGRFYSSAIAFVDYIYKSWFALQKQRQHKIERKERWLSMLKSDGELEQESRCSLDVIRAKAAELLTTVAVQSDSNQNQPTKSNKGSKTKNGKADEVSPTLFNKLFEAYEQTTDTLTRCALAYLLKNGCQVNELEEDSKEFARRRRSKEIEIERLKEQQKSQIPMGRNLTGIPSLETSEIVIHNASKNQGEAKAGQAVLLRKSNCVPFPINYGSSTDLSWFKNDKGRICVKFNGLGKHSFEIYCDRRQLHWFQRFLEDWQIDHDNKDQYSTSLFALRSARLLWSEGKGKDDPWNKHHLTLQCSIDTRAWTAEGTELIRSEKIAAADKQLSNQEQKGELNEKQQDYLQRKRSQRERLNNTLPRPSKPLYQGQSSILVGVSFGLDEPATVTVVDATKGKVLAYRNIKQILGKNYPLLTRQRHQQQSLSHKRHKAQKRSAPNQFGESKLGQYVDRLVAKEIVAIAQTYQAGSIVLPKLSDMREIVQSEIQARAEQKVPGYKEGQQKYAKQYRVSVHRWSYGRLSQCIHESAAKVGIVIEIGRQPIRGSPKEKARDLAIAAYYNRIITQS

SEQ ID NO: 669 - protein, 640 aa, unidentified
  source 1..640: note = MG64-107 effector
MSFITIQCRLVVGETIRRKLWDLMVNKNTPLVNELLKQVTQHGDFETWQREGKVSEKPVQDLCKPLRPDPRFENQPGRFYTSANLMVTYTYQSWFALQKKRCRRLDGMRHWLDVVKSDIELVQTSGCDLERLRAKAQAILGQLSAEESSSKARSPKNKKKQNSKADRDLMGRLFLMYETAEDVLSRCAIAHLLKNDCQVNELEENPEKFADRIRNKQKAIEQLEAKLTSRLPKGRDLTGEEFLETLAIATEQIPENEVDQILWQAKLLAKPATLPYPIVFGSQTDLRWSMNEKSRLCVAFNGVEKFIPELKQTPFQIYCDQRQLPIFQKFLQDWQAHRANEATYPLSLFLFKTASLGWEQGKGKGDPWQVNRLTLHCTINPDLLTAEGTEQVQQQSIAKFETRLSKVTAQETLTEAQQGSIKRQRSSLARLQNAPQRPSKPQYQGFPEIMIGVSIGLVCPITVAVINLKTGQALTYRSTRQLLGDNYRLLNRQRQQQQHHTLKRHKNQTKGYIHQPSESELGQYIDRLLANSIIKLAQQFQASCIVLPQTKNLRERLSAEINARAEKKSDSKQVQDKYAKEVRMSIHRWSYNRLLTAISTQAEKTGLAIETIAQPLQGTPQQKAKDVAIAAYHFRQVSSN

SEQ ID NO: 670 - protein, 637 aa, unidentified
  source 1..637: note = MG64-108 effector
MSFITIQCRLVAPEETLQHLWELMEKKNTPLINELLEQLGKHPDFETWMQKGKLPTGVVDTLCSSLKTNPCFAGQPGRFYYSASTLVDYIYNSWLALQQKRQRQIEGKERWLSILKSDGELEQESGCSLEVVRAKASQILTQVATQSDSKQNQPPKRKKTKKGNANRPPSTVFNQLFDSYGKTKDSLRRCALTYLLKNDCQVSEVEEDPEKLAHRRRKTEIGIERLKEQLKSRIPKGRDLTGQKWLEALEIANHNVPKDEDEAAAWQAALLRKSSSVPFPINYGSNTYLTWFKNEKGRICVKFNCLGKYPFEIYCDRRQLHWFQRCLEDWQIDHDNKGQYHTGLFTLCSARLVWLEGKEKGFPWNVYRLTLHCSIDTRAWTAQGTELIRSEKIAAVDKEIRNKEQKGELNEKQQERLQRKYSERQRLNNTFPRPSKPLYQGQPSILVGVSFGLEKPATVSVVDVTKGNVLAYRTVKQLLGDNYKLLTRQRQQQQSSSHQRHKAQKQSAPNEFGESELGQYVDRLLAKAIVAIAKTYQAGSIILPKLSDMREIVQSEIQARAEQKVPAYKEGQQKYAQQYRVSVHRWSYGRLSQCIHESAAKVGIAIEIGQQAIRGSPQEKARDLAIAAYHARIATLS

SEQ ID NO: 671 - protein, 637 aa, unidentified
  source 1..637: note = MG64-109 effector
MSQITIQCQLVASASTRQQLWLLMAQKNTLLINELLQQVGQHPDFETWRQKGKLQAGIVKALCQPLKTDPRFIGQPARFYSSAIAVVDYIYRSWLALQKRLQYQLEGQTRWYQMLKSDAELIEICGGSLETLRSKAAEILAQLAPESTSVDPQPTKGKKSNKRKNSSNNPNLSAALFEAYRQTEDILSSCAINYLLKNGCQVSEKEEDPEKFAKRRRSVEIRIERLKEQLASRMPKGRDLTDEKWLETLLVASSTVPNSEFQAKSWQDNLLRKSSLVPFPVAYETNEDMTWFKNSKGRICVKFNGLSEQTFEIYCDFRQLNWFQRFLEDQQIKRNSKSQHSSSLFTLRSGRIAWSEGEGKGDPWNIHRLTLYCSVDTRLWTTEGTEQVRHEKADEITRIITKTKEKADLNEQQQAFIKRKTSTLARINSSFPRPSKPVYQGHSHILVGVSLGLDKPATLAVIDAIANKVIAYRSIRQLLGDKYQLLNRQRQQQHQNAHKRKIAQRQGIPNQFSESELGQYIDRLLAKTIVAVAKAYQAGSIVLPKLGDVRESIESEIKARAEQKCPDLVEVQKQYAKQYRSSIHRWSYARLIDSIKSQASQVGILIEEGKQPVRGSLPEKARELAITAYHSRLNTKS

SEQ ID NO: 672 - protein, 720 aa, unidentified
  source 1..720: note = MG64-110 effector
MNMPMFTIPCRLCASEETRRDFWQWMEKYTLLVNELLEKIAEHPQFQEWQKKGDISRKAVREILNPLEKNPCYEGLPRRFYTSAELISCDTYKSWLALQQQRHFQLIGKKRWLQAVESEFELSAITDFKPDQVCAKAGEIREEALQSLNRQGSKHKSLMGVLLDMHGNTAEAPLNRRAINHLLINELQVTDKEQNLDELSKRLDKKRVEIRRLEEQLTSRLPKGRDPTGQRYLQMLCHITALPELSDDLEKLEAELDRWSQQQQLPLGKELLYPIRFDSSSDLYWLLKSQETSNPSETDNQIHQELPKSEKPQKKHRQQSKERIHVQFKGTKDYTFKIQCDRRQLPLFRQFLIDYQTYKQLPEAERFSEGLFALRSAKLIWRKDDTIHGSNKNRGINKQDDQLKPWNTHRLHLHCTVDRRLLTAEGTEQVREEKKREVIKKLKGQDQLEESQLQELGLTKNQISDVKRKCSTLNRLENYSPSRPSTQPYEGQPHIVMGVSFSRRQPVAIAVVNVETQEVLECQSAKEILNRGEAQYICRHGKKEPLIADGAERQHPNGGKLYIRKRKRVQGKPYRLVQQLHQRHQHNSRQKVQQQQQNCYREDNSDSNLGVYVDRLIASRIVELALRRKAGTIVIPQLKGIRESVESDIRAQAERQFPHDKERQKEYAKHYRVSFHGWSYQRLSEFIKECATREGMAVIVRKQPCGGDMEQKAIAIALSP

SEQ ID NO: 673 - protein, 745 aa, unidentified
  source 1..745: note = MG64-111 effector
MAIFTVQCRLDAWGETPESREAIRRYIWEFMAETYTPLVNELISEVAEHPAFKTWQQEGSLDSGALMTLWKSLKKEPRFKGLPDRLLVSARLMVHYTYDAWIALQNEKQTKLDKNIRWVEVVKSDAELVQISGCNLDTIRARAKELLAQINTQNQTQQTTDRRVRKRKASNSSTANQAKSQFIPEAKQNFNGSAEENNKSTTNQVPEIKYKTFDTLFNTYYMSSDILQKCAIAYLIKNKLKVNNKEEDLKKFTKIVRQKKKKIERLQEQLKSRLPKGRHWIGNEFLDNLEAFGIPESETEWFSLQSALLREHNFLPYPILFGSSDDLSWSKQLKISNNSNLIKENSESEKIRERICVQFKGLKEVIFEISCDRRQLPLFQQFLKDWTIYSQNTKEHTSSLFLIRSATLIWEDTKKMKNRQKRQNENKIVNQKSIDTQQEELKKIFQKEIDGEEQPWNRYQLFLHCTVASEFLSKEGTQQLGQKKQELALKAIATLEQKILELEKEGKSTKNDRESFSRKQGTVRRLNNLDNPFERPSRPLYQAQPNILLGVSLGSSKLATATVVDVTTEKVLECQGVRCLLGDNYKLLTRKRYLHEMHSHLRSKAQKRGAKNLLREAELGEHIDRLIATAIIALARKYQASTIVLPNMKDYTEKKQSEIEAFAEQECSGWKCVEKRFTKAQSVKLHRWSYSRLSKIICQQASKVGIAVEIGQQPRHGSSQEQGRAMAIETYHSRKNSLKSKNLRS

SEQ ID NO: 674 - protein, 662 aa, unidentified
  source 1..662: note = MG64-112 effector
MNMSMFTIQGRLCASEETRRYFWEKMEKYTLLVNELLEKIPQQSQFQEWEKKGNISRKIVREILSPLKDNHSYAGLPARFYTSVELISCETYNSWLALQQKRLFKMLSKKRWLQAVESEFELLATTDFNPNAVFAKARDIQNEAMHRLNGQGSKQESLIGILLDMHDDTAENPLSRRAINHLLINHLKISEEEENLDKLSERLDKKRVEIRRLEEQLNSRLPKGRDPTGQRYLQILSYITSLPELSNDPQKLEAELDKLTIQQQLPLFNELPYPIRFESSDDLYWSVLSQDSPNSSHTEHGVHQELPKSEKLQTKHRPQPKERICVQFKGTQNFTFKIQCDHRQLPIFRQFLVDYQTYKKLPETERFSQGLFALRSACLIWRKDDRKHGSKKKRANDKQEDQLKPWNTHRLYLHCTVDRRLLTAEGTEQVCEEKKNRAIQALKGKQELEVSELQKLGLTKAQISDVERKRSTLNRLENNPYPPRPNTIPYEGQPHIVIGVSFSRNEPVTIVIVDVEKQEVLERQSAEELLNRGEPQYIWRNGKKEPLLRDGTERQHPNGGKLYIRKGKYAQCKPYRLVEQLHRQHQRNCKQRIQEQKQDRYRETNSDSSLGLYVDRLIASRIVSLALQRKAGTIAIPQLKGVRENVESDIRAKAERLFPNEM

SEQ ID NO: 675 - protein, 696 aa, unidentified
  source 1..696: note = MG64-113 effector
MLTPDLRNFMSIITIPCRLVSREFSRYCLWKLMADQNTPMINELLALVGNHTEFEAWQQQGKLPAKLVKNLCDSLKSDPRFEGQPSRFYSWAVATVNYIYKSLFAIQQKLRRDLERKSYWLSRLETASSFIQTNKLSWSDLYRKAEEILATAIAQNSTDEAQQRNGKKEKKDKTPRKLATILFGLYNQTEDPAEQCIITCVIANGCCLRKPEEDLPDFTELRREKEIEIERIQEKLKTSRLPKGRDLTGKDWLEALELATFHQLDNEQHQAVQANLLRESSSLPFPVSYESPPDFTWSRNDKGRICVKLSGLAKHHTFEVYCDRRHLPWFERFLKDQETRHDSEDKLSTSLYTLRSGQLIWKEGEVSKSSSVAPSLFKWLFYYGLQILCSLPEVSLWQSGELKLGLLAYLLLKNRKREPWHVHHLHLHCAVETLLWTAEGTQQVANEKAARADKTISKMEEQQKKGELNEAQQASLIRTRSMRDRLNNPFPRPSQPLYQGQSNLVVGVSMGLDKPATVAVVDVNTGKVLTYRNVKQLLGKNYKLLNQQRQRQHRDARRRHKAQKKDAPNQFGASKLGQYVDRLLAKAIVAIALTYKASSIALPKLGEVSERVNSQIQARAEKECLGHKKSYQKYGKQYRSRIHSWSYGRLIENIQQAAAKVGIAIVEGQQPSKGTPQEKAGNVALSAYQNRMRAVS

SEQ ID NO: 676 - protein, 644 aa, unidentified
  source 1..644: note = MG64-114 effector
MSFVTIQCRLVVSESIRCQLWNLMANKNTPLVSELLKCVSQHDEFEAWQRSGKVPVKTVRELGEPFKMDSRFEGQPGRFYTSASLMVAYTYKSWLALQRQRRQRLDGKLRWLNIVKSDAQLVQDSGCDLDTIRTKAGEILAHLNAQASPEPPRSTKQKRKKKKQQSLAADPRLMNLLFQAYEAMEDTLSRCAIAHLLKNDCQVSAQQEDPEAFTQRIHRKKKEIERLQAQLLSRLPKGRDLTGERFLESLAIAVGKVPDDAIEQLLWQAKLLAKRAAMPYPILFGSQDDLRWSINERGRICVAFNGLDKAIPELKRNPCQIYCDRRQLPLFQRFLADWQTFQANQDTYPLGLFLFQTGLLGWEEGQGKGEPWSVNRLTLHCTLDTHPLTAEGTEQLRQAEIARLTEKLSAVQNPEALTQNQQAWLKRQHSTLARLQNPPQRPSKLVYQGDPEILLGISIGLAHPATAAVVNVRTGKVLEYRTTRQLLGKNYRLLNRQRQQQQLHALQRHKHQIKGRTSQLGESELGQYVDRLLAKSIVELAHQFQASCIVLPQTNNLREHLAAEISARAEQKSDSKQAQDKYAKQFRISIHRWSYARLLMTIRHQAEKAGISIETGSQPLRGTSNKKAKELAIATYHLRQVACN

SEQ ID NO: 677 - protein, 705 aa, unidentified
  source 1..705: note = MG64-115 effector
MTMPMLTIQSLLCASEETRQLFWVWMEKYTFLVNELLEKIPQHSQFQEWQKKGDIPLKTVREILSPLKGAPQYAGLPGRFYTSAELMSCNTYKSWLALQKERQIRLTGKKRWLHAVESEFELSAITEFNPDKIRSKAGSLLKKATQKLEKEGGKQKELIEILLEEHDKTAKNPLSRRAINHLLINDLKISEEEQNLSELSERLEKKKVEIRRLEEQLTSRLPKGRDPTGQRYLQILCHISASPELRDDPEKLEAELDKLTEQQQLPLFNTLPYPIRFDSSGDLHWSLENLKSKHWKHPKERICLDFKGVKGRIFKIQCSRRQLPVFRQFLNDYQAHESLLEEERFSEGLFALRSACLIWHKDEMRHRSKKKKQIDQQEDQLKPWNTHRLYLHCTVDRRLLTAEGTEQVREEKKKKTIEALKGKENLEESHLKQLGLNKNQISSVKRQKTTLNRLENYSPPPRPNAKPSEGQSHIVVGVSFSRYEPVTIAIVDVEKKEVLECQSAKELLNRGEAHYIWRNGKKELLKIDGTERQHPNGGKLYIRKGKQVQWKPYRLVKQLHQRHQHNWRQRAKQQQQNRYRQDNSDSNLGLYVDRIISSEIVELALKRKAGTIVIPQLHGIRESIESDIRAQAERRFPHDKERQKEYLKDYRSSFHRWSYGRLSECIKERAQAESIAVVVQKQPSGGNLEQKAIAMALSSYNVKIS

SEQ ID NO: 678 - protein, 721 aa, unidentified
  source 1..721: note = MG64-116 effector
MNMSMFTIQGRLCASEETRRYFWEKMEKYTLLVNELLEKIPQQSQFQEWEKKGNISRKIVREILSPLKDNHSYAGLPARFYTSVELISCETYNSWLALQQKRLFKMLGIKRWLQAAESEFELLATTDFNPNTVLAKARDIQNEAMHRLNGQGSKQESLIGILLDMHDDTAENPLSRRAINHLLINHLKISEEEENLDKLSERLDKKRVEIRRLEEQLNSRLPKGRDPTGQRYLQILSYITSLPELSNDPQKLEAELDKLTIQQQLPLFNELPYPIRFESSDDLYWSVLSQDSPNSSHTEHGVHQELPKSEKLQTKHRPQPKERICVQFKGTQNFTFKIQCAHRQLPIFRQFLVDYQTYKKLPETERFSQGLFALRSACLIWRKDDRKHGSKKKRANDKQEDQLKPWNTHRLYLHCTVDRRLLTAEGTEQVCEEKKNRAIQALKDKQKLEVSELQKLGLTKAQISDVERKRSTLNRLENNPYPPRPNTIPYEGQPHIVIGVSFSRNEPVTIVIVDVEKQEVLERQSAEELLNRGEPQYIWRNGKKEPLLRDGTERQHPNGGKLYIRKGKYAQCKPYRLVEQLHRQHQRNCKQRIQEQKQDRYRETNSDSSLGLYVDRLIASRIVSLALQRKAGTIAIPQLKGVRENVESDIRAKAERLFPNEKELQKKYAKDYRDSFHRWSYARISECIRECAKKEGIAVVLRKQPSQGNLEEKAKAIALSS

SEQ ID NO: 679 - protein, 717 aa, unidentified
  source 1..717: note = MG64-117 effector
YTYKSWLSSHKRDYNKLQGKKKWLAIMDHDLEVAKSTDFSSQLIQTKAAEMLSQAYAQRENLQQNQTQQIKKKPSEKLENCPSIMTILFGMYDKSQNSLECRALAHLLKNGCQVNEEEEDTEALKFRLEKKRIQIARLEKQLESRLPDGRDPTGERFASNLDNAIALPEYSTFISSKFWANWYQALQTNPDNNSQLLILFLSFISYKQNIAAEFDAWAQEEPQRLALTSTIYETLPYPIRFGSTEDLYWSSEQVDPNPKATREERHARQCPKRRRKPKHKQKVNERICVRFKGKGLENHRFRINCDRRQLPVFKQFLTDRETQKQRKKAEKFMGGAFTLRSACLVWKKDTEGLHRKRTSATSLLWMKFLFAIQWKETITDKELDSWSISLLCINTTFPWQTHHLYLHCTIDSRLLTAEGTEEVIAGKAAAAQQYLDESKLKASTSKRQAVERMDEEKQQTPQAKGETTAKKTKTTLARSKNPPPARPSRPVYKGQSYVEAVVVVSRFAIVAIAVVNTQTQEILEFQEVKDLLTEHRSDVLEKRLKKHQGMLKGRQSLEQLQLQQYRLVKRWRKHRKKNLTQRQEEQKRGLYKRSNQESNLAQYLNRLLAKRIVQLCQTYQVGTIVLPELGNIRENFECEIQAKARQKFPDDNVQLQKQYAKEVRMRVHRWNYKNLLHAIRQCATHTRIPVTTRWQPKEGTLRDKATIMTRPVPIGVP

SEQ ID NO: 680 - protein, 739 aa, unidentified
  source 1..739: note = MG64-118 effector
MLAENEFIIWLGMPMFTIQSRISASRETRQYFWEVMHKHTLLVNEVQEKIAQHSQFQEWQKKGNISRETVRGILAPLKENPSYTGLPGRFYSSAELISCYTYKSWLALQQQRQLRLLGKKRWLQAVESEFELLATTNFNPDEVRVKAREIHNEAIQKLNGGGRKYKAPSLIGIVLEMHDRTAEAPLSRRAINHLLINNLKISEEEQNLEKLSEHLGKKRKEIERLEEQLISRLPKGRDPTEQRYLETLYQVTALPELSNDPEKLATELKTLTVQQQLPLFKELPYPIQFGSSGDLYWSVETQGTSNPADAENGINQELPKSKKRQKKCCKRPQERICVQFKGVIDHTFKIQCDRRQLPIFRQFLIDYQTHQELPDEERFSEAEFALRSACLIWRKDDTGQSSNKKRTSDEQEDQLKPWNTHRLYLHCTVERRLLTAEGTEQVREEKKKEVIKTLKGQEKLQELELEQLGLTKTQIEYVRRKRSTLNYLENNSPPPRPNAKPYEGQPHIVIGVSFSRHEPVAIAVVDVKKEEVLECQNAKELLNRGKAQYIWRNGKKEPLIKDGIEQRHPNGGKLNIRKGKRVRRKPHRLVQQLHQRHQQNSRRRSEEQKQDRYRSSNSDSNLGLYVERLIASKIVELALQRKASTIAIPQLKGIRESVESDIQARAERLFPNEKERQKEYAKHYRASFHRWSYNRLSECIKECASSEGIAVVIRQQSSVGELEQKAIAIALSFSNVKTS

SEQ ID NO: 681 - protein, 676 aa, unidentified
  source 1..676: note = MG64-119 effector
MSRNSKKNSTSPILQTIRCHLHASEDVLCKVWEEMTQKNTPLIVQLLSGVSEQPEFEVNKENGKISKPEITELRRFLTKDSDLEKQSGRLRSSADTFVTEVYSSWLTLYQKRKSQKEGKEYFVKNILKSDVELIAESNCDLQTIRDKAQEILAQPEKILKQIVASDENSKQTNSNQKEDKKKSKKNSSTKQKSNIVAQQKDNDSKTLTNILYEIHKKTQDVLTRCAVAYLIKNHNKTSDLKEDIEKLKERRTKKEVEIKRLEKQLQDNRLPNGRDITGTTYAEAFDNLIKQVPKNHEECTTWIADLLKKISPIPYPINYLYSDLSWYKNDKEQICIYFSGWAKYHFQICCNKRQRHLFERFLEDHIAFEASEKGEEKLSGSLVTLRSVQLLWQQAEGKGEPWKVHKLALHCTYDARLWTAEGTEEVIKEKTDKAQKKVSNAEKNENLDNNQQTQLNKNKSSLSRLSNSFNRPSKPVYQYQNNIIVGISFHPVELATVAVIDINTHKVLDNKTVKQLLGDDYHLLSRRRRQQVHFRKEREKAQKKDASCNIGESELGEYVDKLLAKRIVEIAKHYRAASIVLPKLKDMREIRTSEIQAKAETKFSGDINAQKLYIKEYNHQIHNWSYNRLQECIKSKAAQLRICIEFGIQPNYGTLQEKARDLAFSAYQSRTNDIGK

SEQ ID NO: 682 - protein, 621 aa, unidentified
  source 1..621: note = MG64-120 effector
MSQKTIQAQLVATESSRQVIWQLMAERNTPLINALLEQIATSSGFEEACSKGSISQSIVKECCQKLREDSQFSGQPGRFYTSAIVTVSRIFKSWIHIRRKIIYQLEGQTRWLLILQSDEELTEACNCDLDILRSKAAEVLAKVESQTHQKGQFNILFELYRDAKDSLTSGAIAYLLKNGSKLPDKPEDPKKFVKRRRKAEIRAERLTTTLKRLRIPSGRDLTGQKWEETLAVAASSVPENEDEAALWQAVLLSETKKLPFPILYETNEDLTWFLNDEGRLCVNFNGLSEHSFEIYCDQRQLHWFKRFFEDQETKKASKNQHSSALFTLRSARLAWQKGEGKEELWNVHRLVLSCTVETRLWTAEGTEQVREEKATEYAKVIAGTKAKGNLNKNQEKFVQSREKTLALIKNPFPRPNRPLYKGQPTILAGVSYGLDRPATLAIVDITTGKAITYRSIRQLLGDNYDLLNGYRLRQQRNAHRRHNRQRTGASNQIQESDQGEYLDCLIAQAIVSTAQDYQASSIVLPDLGNIREIVQAEVQARAEQKIVGYLEGQKHYAKQYRASIHRWSYSRLNEKIQGQSAQIGIAIEQTKQALQGTPQEKAKNLVLEAYNSRQESSLTRK

SEQ ID NO: 683 - protein, 707 aa, unidentified
  source 1..707: note = MG64-121 effector
MSIITIRCRLVAGVKQQIDKKSIKNFSAEDRALLQKLLDDKSHADRDNPKDKEKIFLAQSSEAVRQNLWQLFLTSSALIDELLDRLSQHPNIHTWQQQGNLPDDELKACWLELKASPLYDEKLPGRFFSSVQSMVKNIYASWLALNQQKQRRLNGLNRLTEIAYSDEALLEMCDLTFTQLQANAESMLAEIDKEIAGSEKPLSRINLLFQKYTELPDSDILGRSAIAYLIRHGCKIESKIEPAAEFKKWFKTKLKQARRLENQLAGHFPRGRDLNGTAFLNVLEIATNDEPQDNQELMLWQSQILRDPSSLPHPIEFNSNTDLRWLKLDRKQYKCQRVASGESIESIELTQRLFVEFNGLTRGTNYVFEVYCDRRQLAIFQQFFNDDRLSRNSSSDEKYSSSLFTLRSAHLLWDRNESQDRHRTLATQTADEPWNSNQLYLHCAIETKSLTAEGMREIKQQKTQKVNNTLVKQSKNIDPSIDQQQSYRKNQTSLALLDRPLPRPSRPKYQGNPQIIVGLIFDPVRPIYLAVVDVTTGKTITCRSTRQLLGDKHPKLSEYRLKQQQNSSLRRKQNQQGQFNQPTESTQGEHLDRLLAKAVIRVAQEFKAASIALPPVNNSIEKNQSELEAYAEEEIPEDIVTQQQLTRKTSVVIHKWSYNRLSGYIRNNATKLDIAVETASSPSPGTPLQQAAESAISAYNSRKHIKK

SEQ ID NO: 684 - protein, 634 aa, unidentified
  source 1..634: note = MG64-122 effector
MKDVGTLLIEIKKPVGDVPLVYKRLRALTRSGAQALNMTMQDSHPNAVAQLRAVWDGEAKQTRTSSEDWRARVRTVLARNWNARLERERLDRKAEPDAEMVYAGINGSALAEETATNITSQFNGENMKEMIALRKGFPEFGIASSFYCGGRYCAVVGTLRETLKRTCATKDDADAAVKELGEGHFARRAGDKWQVVHEDSARVALPLWGTGKKVTELIIGPAGGHVRSTWRQLVAHFERRDEIVQAEKELDAIYRTEAEKAALASLKAVKKTERDKTKRREIDGQIRAVVEASNKRMQSLRDPIEAKLYGLTKIGRIGVVWKERRRKWFVTISYTRYTPSIETKGQAAAVNFGINVFLQAFAADESAFHVDGAQILHKRLAYNAARKRIQKSKRQFGRGSRGRGKRRRELPLTKRQGDETNWTQTFIRQLASDLIAWCKRRGVADLYLEDLSGVRDEFEQATGGQAHPEVKRRIHSWPYAETRMAIVREGTQHGVRVHAKGSRFVSQRCPSCTHTVPENVQEVTIPGPVLPLQSLGKGRWREVPYQRFQDSESKAAQYDLYRRQDKTYRFECVACGMKGQADIVACLNHLQDLGVVPAGKPTPPGTPTPLAKMQEAARASVSNDKRKKVRRTGT

SEQ ID NO: 685 - protein, 634 aa, unidentified
  source 1..634: note = MG64-123 effector
MKDVGTLLIEIKKPVGDVPLVYKRLRALTRTGAQALNMTMQDSHPNAVAQLRANWDGEAKQTRTLSEAWRGQVRTVLARNWNARLERERLDRKVEPDAEMVYAGINGSALAEETATNITSQFNGANVGEMIALRKGFPEFGIATSFYCGGRYCSVAGTLRETLKRTCATKDDADAAVKELGEGHFARRAGDKWRVVHEDSARVALPLWGTGKKVTELIIGPAGGHVRSTWRQLAAHFDRRDDIVLAEKQLDAIHRTEAEKEALASLKAARKAERDRKKRREIDGQIQAVVGAMNKRMQSLRDPIEAKLYGLTKIGRIGVVWKERRRKWFVTISYTRYTPPIETKGQAAAVNFGINVFLQAFAADESAFHVDGAQILHKRLAYNAARKRIQKSKRQFGRGSRGRGKRRRELPLTKRQGDETNWTQTFIRQLASDLIAWCKRRGVADLYLEDLSGVRDEFEQATGGQAHPEVKRRIHSWPYAETRMAIVREGTQHGVRVHAKGSRFVSQRCPSCTHTVPENVQEVTIPGPVLPLQSLGKGRWREVPYQRFQDSESKAAQYDLYRRQDKTYRFECVACGMKGQADIVACLNHLQDLGVVPAGKPTPPGTPTPLAKMQEAARASVSNDKRKKVRRTGT

SEQ ID NO: 686 - protein, 714 aa, unidentified
  source 1..714: note = MG64-124 effector
MSVITIQCRLVASEETRRYLWSVMVEKNTPLINELLKRVKQHPDFEKWRRRGKPSKEAVRELCEPLRNDPQFNGQPGRFYTSAIATVQQIYESWMATQLGLQRSLDGKVHWLEVVESDAELVTQSNVDADAIRAKAREILLDIASGCLPQKTQEKKAKASQNKEKPRQGKKQQKEKPEVPQNLVGVLLDFYDETKEVVKRRALIHLLRNDCEVNEKEDAKKLIDKLNNKRAEIERLKERLEGRLPKGRDLTDQDFLEALEIATSIPADDTLSEFLAWENKVIPNLPKLVKAPKSMPYPIYYETNTDFNVWKRNEKGRICFELNGLSNHIFEVYCDRRQLHFFEQFLRDYETKKTGGKQHSTGLFLLRSAQLLWCEDKTRIKKKKVKVQNPLGGKAKKKAEKTKIVEPWNYNYLSLHCAVDTRLLTEEGTQQVREELKEDIAETLERQRTKLSNLSDEQVEEKKNLRQSIQSKESTLSRSSGSFSRPSRIPYSQFSRSNVLVGVCFEFHNLVTVAVVDASQNQVLAYRSTRQLLTNERVEIPEAAEGSAKRVSRRLRYQNYRLLNRHRQRQQRNARQRREDRKRRIYRNSPKSQSGQHLDRLIASSIISLAQKHRAGSIVLPETKGLLERTEGKVRALAEQKVPDYKQGQEKFAKQHRKRYNRWNYARLLQTVQQQAAKLGISVELGRQISEGSSQEKARDLALAIYHSRQVVDA

SEQ ID NO: 687 - protein, 635 aa, unidentified
  source 1..635: note = MG64-125 effector
MSQITIQCRLVSSPGTRQQLWTLMAERNTPLINALIEQISQHPEFEIWRRKGKISSTLVAQLCKSLKTEVRFNGQPARFYTSAEHAADYIFKSWLAIQKQLQQRLDGKLRWLEMLKSDEELAQAGEVELNVIRDRASAILSQLQPTTPNDESPSHTKKKGKQIKKKVASTDRSLVSQLFDRYRDSKQVLERCAIAFLLKNGCKIPQDNEDPQKFAQQRRKAEIQVKRLQEQIESRIPHGRDLTGASWLNTLETATQTVPKDNAEAKRWQNRLLTNPSVLPFPLIFETNEDLVWKRNAKGRLCVHFNGLSDYTFAIYCDNRQLHWFQRFLEDQETKRSSKNQHSSGLFTLRSARLAWQVGEGKGNPWDVHQLTLYCTIDPRLWTAEGTEQVRQEKAAEVAKKITQMESKGDLLVTQQVYVKRLNSTLTRINTPFDRPSKPLYQGQSHIVVGLSLGLEKPATVAVWNADTNQVLAQYGIRQLLGENYRLFTRRRTEQLKTAHQRHKAQKREAPKQLGESELGQYVDRLLAKSIVTIAKTFKSGSIAVPKLGNIREIVEAEIKAKAEEKCPGFVEGQRKYAKQYRTNVHRWSYGRLIESIRSQATKLGIVIEEAKQPLTGKSEEKAKDVAIAAYQART

SEQ ID NO: 688 - protein, 612 aa, unidentified
  source 1..612: note = MG64-126 effector
MKDESTSVGASSLLQHGATPRPEGEEVSGHVPLRRKEASPETLAKSGTSVPTRSDPAATPEGGEEAPTDVDSGGGSGGHGRSAKRRPKGKRTSIDGKPPDGIVEGEPLPEPTITRATTKFSIRLDALSENLNRARKKGQKRARSLAEDEAREALIVASRYATTAQNVGMRLLYRADSDVLDAFMAEHGRLPIPGEVIWPSAKAYRADSPVYVYRRMVMAVPELSSSIAATLSKKVVEKWASERVDTLVRQQRSAPHYRLGGPLPIPAQVTQWTWDPSSKRAMVDVTLFSTKKRKGVHRVTIPVEARDDRQAKELEMLALGDGNPAVGWRPGEVTIQRDRLRPQKWFLRCAYTRVAVARKEGVAAAINLGMRCLLAFYVEGGPSDIYEARDIEAYLRGIQRRRRDRQNVYRWTDGSRRGHGRPRALQSIEVLAERAANYRKTRMQTLARRFARRLVEMGVNVLYVQKFDGIRDALPELLMASMPRSQWIWERIQEWPYSEMQARIVTCCQDEGIRCVEMAAQYNSQRCPVCGFVSKDLRDLVNWRLKHAGCINRHLDVGHAMNAIARGAATDPDGSRKIKGLAEWNLNLDETARETSGKKAKEKGNGETGGDG

SEQ ID NO: 689 - protein, 692 aa, unidentified
  source 1..692: note = MG64-127 effector
MSIKTIECRIVAEPESLKALWIIMTQKNTPLINELLAQVPNHPDFEQWLEYGNLPQKPIKELCAPLREKEPFANQPGRFYTSAIALVHYIFKSWLNVQKKLRQRIEGKQRWLDMLKSDRELQDESGKDLAEIRLLAVEILQQIEQHLACQKVNLKKKQKKRKKTKSKKTSSTIFNSLHERYDKTDDYATKCAIVYLLKNGCQISEAPEDEEDPEEYALGRRKKEIEIENLQKQLRSRRLPSGRDLKHEVYSQALEAIEDNHVPEDNTESKLLQDNLSRKSVAVPYPVAYESNTDLTWLEDKNGNLSVKFNGLGKHEFEIRCDTRQIDWFRRFLEDQKLKEKSKQDKKKGLRDSEISSALFGLRSGRILWRKGEQKSDRRKKTLDRAFFLLSLSKDYKLALALLQGYGQYKRRLRQEQPWTLHRLYLQCSIETKLWTQEGTKLVAKQKNTKSNNTITQTKEKGDLDSEQQKHIKRKQAQLANLKNIPQRPCKDLYKGNSSMILGVSLGLEKPVTVAVVDVVSGRVLTYRSAKQLLGKNYKLLNRQRQEKLRLSIKRHKSQKRNASNNFGESNLGKYVDRLFADAIIKLAQSFKVSSIAIPEVRQIREITTSEVRAKAERKIKGYKEGQKKYAKQYRVNVHNWSYGRLTNFISQKAGIYGITIEKARQPLGSSNQEQARNIALSVYNSRLENVV

SEQ ID NO: 690 - RNA, 38 nt, synthetic construct
  source 1..38: mol_type = other RNA
ggtcgcaatggccgttttggccggagaagggatgaaag

SEQ ID NO: 691 - RNA, 37 nt, synthetic construct
  source 1..37: mol_type = other RNA
gtcgcaaaaccctaccctggccagggtgggttgaaag

SEQ ID NO: 692 - RNA, 37 nt, synthetic construct
  source 1..37: mol_type = other RNA
gtttcaacaactatcccggctaggggtgggttgaaag

SEQ ID NO: 693 - RNA, 37 nt, synthetic construct
  source 1..37: mol_type = other RNA
gtttcaacacccctcccagcgagaggcgggttgaaag

SEQ ID NO: 694 - RNA, 36 nt, synthetic construct
  source 1..36: mol_type = other RNA
gtcgggatggatctggagaaggaatggtgttggaag

SEQ ID NO: 695 - RNA, 36 nt, synthetic construct
  source 1..36: mol_type = other RNA
gtggcgacgggtgaggaggccggatcgggttggagg

SEQ ID NO: 696 - RNA, 37 nt, synthetic construct
  source 1..37: mol_type = other RNA
gtcgcaatcgctcttccagaaatgggggggctgaaag

SEQ ID NO: 697 - RNA, 37 nt, synthetic construct
  source 1..37: mol_type = other RNA
gtcgcaatcgctctctcgaacagggggagattgaaag

SEQ ID NO: 698 - RNA, 37 nt, synthetic construct
  source 1..37: mol_type = other RNA
gtcgcaatggccctcctagtgataggtgggctgaaag

SEQ ID NO: 699 - RNA, 37 nt, synthetic construct
  source 1..37: mol_type = other RNA
gtttcatcaaccctcccgccttggggtgggttgaaag

SEQ ID NO: 700 - RNA, 37 nt, synthetic construct
  source 1..37: mol_type = other RNA
gtttcatcgaccctcccgcatttgggtgggttgaaag

SEQ ID NO: 701 - RNA, 37 nt, synthetic construct
  source 1..37: mol_type = other RNA
gtttcatcaaccctcccgcctatgggtgggttgaaag

SEQ ID NO: 702 - RNA, 37 nt, synthetic construct
  source 1..37: mol_type = other RNA
gtttcatcaaccctgccgcaacagggtgggttgaaag

SEQ ID NO: 703 - RNA, 37 nt, synthetic construct
  source 1..37: mol_type = other RNA
gttgcaatcgccctcccgccaacgggtgggttgaaag

SEQ ID NO: 704 - RNA, 38 nt, synthetic construct
  source 1..38: mol_type = other RNA
gggcgcaacagcccttttagccacgggtgagttgaaag

SEQ ID NO: 705 - RNA, 37 nt, synthetic construct
  source 1..37: mol_type = other RNA
ggcgcaacagcccttttagccacgggtgagttgaaag

SEQ ID NO: 706 - RNA, 36 nt, synthetic construct
  source 1..36: mol_type = other RNA
gtttcaatgaccatcccaactaggggtgggttgaaa

SEQ ID NO: 707 - RNA, 37 nt, synthetic construct
  source 1..37: mol_type = other RNA
gtcgcgatcgccgtttcagccttgggcagattgaaag

SEQ ID NO: 708 - RNA, 37 nt, synthetic construct
  source 1..37: mol_type = other RNA
gtcgcaatcacctgtccgaattagggcaggttgaaag

SEQ ID NO: 709 - RNA, 38 nt, synthetic construct
  source 1..38: mol_type = other RNA
agtcgcaatcggtctcccagagatgggcgggctgaaag

SEQ ID NO: 710 - RNA, 38 nt, synthetic construct
  source 1..38: mol_type = other RNA
gttgcaatagccctcctaaattagggtgggttgaaagg

SEQ ID NO: 711 - RNA, 38 nt, synthetic construct
  source 1..38: mol_type = other RNA
gtttcattagccttcccgcttttgggtaggttgaaaga

SEQ ID NO: 712 - RNA, 38 nt, synthetic construct
  source 1..38: mol_type = other RNA
gttgattagaccttcccgaactgggatgggttgaaaga

SEQ ID NO: 713 - RNA, 38 nt, synthetic construct
  source 1..38: mol_type = other RNA
agtttcaataccccttccggctaggagtaggttgaaag

SEQ ID NO: 714 - RNA, 38 nt, synthetic construct
  source 1..38: mol_type = other RNA
agttgcaatactccttctgacaaaaggtggtttgaaag

SEQ ID NO: 715 - RNA, 278 nt, synthetic construct
  source 1..278: mol_type = other RNA
ccaggtgaatgagtaagatcccccaaggatctaaaccttgagcagtcttctgctgaggttgggcatggttctcattgtggctactgaccttctcgattttttggcaaagcggagcggggcaaaaatcccggtatccttgccaagttcctcatttagtgccagccatgggtttgcgctcgcgctcattgaatcgttgctagtcagcgaagcgcaaaagcgatgcaaacatcaggctttgccaatgcgaggcttggaaaggttgcagcacattgaatgga

SEQ ID NO: 716 - RNA, 265 nt, synthetic construct
  source 1..265: mol_type = other RNA
tttctaatagcgccgcgactcatgctctgcctctgagtcgtgttaaatgagggttagtttgactgccggaaggcagttttgctttctgaccctggtagctgctcaccctgatgctgtcggataagaagcttaggctttttagaagagattaagtctgatagaacattgctttcctattatgacgtaggtgcgctcccagcaacaagggcgcggatatactgctgtagtggctactgaatcacctccgatcaagggggaacccgcc

SEQ ID NO: 717 - RNA, 257 nt, synthetic construct
  source 1..257: mol_type = other RNA
agaataatagcgccgcagtttatgttctttagagccaatatactgtgaaaaatctgggttagtttgacggttgtcagaccgttttgctttctgaccctagtagctgcccgcttctgatgctgctgtcgcaagacaggataggtgcgctccctgcaataagaagtaaggcttgaaaaagtaatagccgttgctagcaacggtgcgggttaccgcagttggtggctactgaatcacccccttcgtcgggggaaccctcc

SEQ ID NO: 718 - RNA, 242 nt, synthetic construct
  source 1..242: mol_type = other RNA
aaaattcaacagcgccgcagttcatgcttgttataagcctctgtgctgtgtaaatttgggttagtttgactgttgttaaacagtcttgctttctgaccctggtagctgcccaccttgatgctgctgtcccttgtggataggaattaggtgcgcccccagtaatagaggtgcgggtttaccgcagtggtggctactgaatcacctccgagcaaggaggaacccaccttaattattttttggcg

SEQ ID NO: 719 - RNA, 334 nt, synthetic construct
  source 1..334: mol_type = other RNA
gattcaacagcgccgcagttcaagctctttgagccgctgaactgtaaaatgggtcagtttggtcgttgtgaaacggctgtactttccgaccctagtggctctccgctcactgactgccatcctggtacaggtttcatgaggaatttgtgtggggatgggaagctgcattggttgagttcgttcttcaaatgtagcgcaggtgcgcgcccagcagaagtgagtcaagccttcattctgtgaaggtacgggagcgttgtctcttcaagtagttgatgctgatgacgtgaccggagcgacagccactgaatcgcctccgatcaaggaggagtcctcc

SEQ ID NO: 720 - RNA, 218 nt, synthetic construct
  source 1..218: mol_type = other RNA
tgtttaatagcgccgcaggtcatgcttttcggagcctctgaactgtgataaatgagggttagtttgactgttgtgagacagtcttgctttctgaccctggtagctgctcaccccgatgctgctgtcacatgacaggataggtgcgctcccagcaaaaagggtgcgggtatactgctgtagtggctactgaatcacccccgaccaagggggaaccctcc

SEQ ID NO: 721 - RNA, 237 nt, synthetic construct
  source 1..237: mol_type = other RNA
acaataatgctggcgacccagttagtgctttaggcacgaacaagtggaaaaggtcagttttacccttgggtgctttccgacctgtgtactgtccgctattcatgctgctgcctaataaggcaattcgccacagcaatgaatagcaccgctctaccgccttaaaaaagcggtacagtaatgcagatgtggcagtccaaatcgcctactgaatacggtaggatctccccacaaattttt

SEQ ID NO: 722 - RNA, 326 nt, synthetic construct
  source 1..326: mol_type = other RNA
aaattaacagcgccgtcgttcatgctcttcggagcctctgtacggtgaaaaatctgggttagtttgactatcggaagatagttttgctttctgaccctagtagctgcccgctcctgatgctgctgtctatcgaattgttcgtccagacgggaaatcttagctctaaatatctaaagagtattttacttctaggatatccagagataagagaggtgcgctcccagcaataaggagtaatgcatgacttgcactagcccttggtaacaagggtgcggataaccgcagtggtggctactaaatcacccccttcatcgggggaaccctcc

SEQ ID NO: 723 - RNA, 301 nt, synthetic construct
  source 1..301: mol_type = other RNA
tattcaatagcgcgtcttgttccccgcgaacagatgataagtgtcagggcagtttaattgctttccgcccttggtagttgtccgcgtctctctagaaattggagagacacgtcctttacggagcttgctgtgtaatcgacgttccttctacggtacttttccgtagatctggtgcagggtcgcgcctagcatcaaggagctatgtttttataaccagtggtacaacgcttctggttataaggatacaggagtacgtgtcgtggcagctacccaatcgccttcgagcaagaaggaatcctcc

SEQ ID NO: 724 - RNA, 306 nt, synthetic construct
  source 1..306: mol_type = other RNA
tattcaatagcgcgtcttgttccgtgagagcagataataagtattagggcagtttaattgctttccgcccttggtagttgtccgcgtctctctaatctttagagaagagagacacgtcgggagcagagcttactctgtaatcgacgttccttctacggaattttttcgtagatacggtgcaggctcgcgcctagcatcaaggagctatgtttttataatcagtggtataacgcctctggttataaggatacaggagtacgtgtcgtggtagctacccaatcgccttcgagcaagaaggagtcctcc

SEQ ID NO: 725 - RNA, 204 nt, synthetic construct
  source 1..204: mol_type = other RNA
atgatactaatgcgccgtggttcatgctcgaatagagccaatgtgctgcgtctgagtgtgggttaggttaattgctttctgacccaggtagctgccagccctgaaggtggtatgcgctttcaccaataggggtgttgatgtacttccgcagcggctactgaatcacccctgagcaaaggggaatccactccaattttttgattt

SEQ ID NO: 726 - RNA, 324 nt, synthetic construct
  source 1..324: mol_type = other RNA
gaattaacagcgccgacccttcatgctcttcggagccaatgtaggtgaaaaatgggttagtttgactatcggaagatagtcttgctttctgaccctagtagctacccgcttctgatgctgccgtctattgaattgtcgtccagacgggaaatcttagctctaaatatctaagatagtatcttacttctaggatatccagagataggagaggtgcgctcccagcaataaggagtaatgcatgacttgcactagcccttggcaacaagggtgcggatagccgcagtggtatctactgaatcacccccttcatcgggggaaccctcc

SEQ ID NO: 727 - RNA, 290 nt, synthetic construct
  source 1..290: mol_type = other RNA
atttgaatgatgattagcttgttgggtcagtttgactgttgtgagacagttatgctttccggccctggtagctgcccgctcgctgactgccatcctggtacgtgttctcttgcagatgcactatggggatgggaagttgcagttgtagaatctttctttaactgtagcgtaggtgcgcgcccagcagaagtgagtccagccttccataaagaaggtacagcctagccctgtagtggtggctaccgaatcgcctccgagcaaggagggtcctccaaatatttttggcaaac

SEQ ID NO: 728 - RNA, 301 nt, synthetic construct
  source 1..301: mol_type = other RNA
tattcaatagcgcgtctggttccctgagaacagatgataagtgtaagggcagtttaattgctttccgcccttggtagttgtccgcgtttctctagagattagagagacacgttacttgcggagcttgctctgtaagcgacgttcctctacggatgtcaatccgtagatatggtgcagggtcgcgcctagcatcaaggagctatgtttttataaccagcggtttacgatctctggttataaggatacaggagtacatgtcatggcagctacccaatcgccttcgagcaagaaggagtcctcc

SEQ ID NO: 729 - RNA, 306 nt, synthetic construct
  source 1..306: mol_type = other RNA
tattcaatagcgcgtcttgttccgtgagagcagataataagtattagggcagtttaattgctttccgcccttggtagttgtccgcgtctctctaatctttagagaagagagacacgtcgggagcagagcttgctctgtaatcgacgttccttctacggaattttttcgtagatacggtgcaggctcgcgcctagcatcaaggagctatgtttttataatcagtggtataacgcctctggttataaggatacaggagtacgtgtcgtggtagctacccaatcgccttcgagcaagaaggagtcctcc

SEQ ID NO: 730 - RNA, 312 nt, synthetic construct
  source 1..312: mol_type = other RNA
ttttgacgcgctgctatagcagctaaatgggtcagtttcagtactttccgtcccaagtagttgtccgcttctgtcaagtagtgcgatacagctttcctgtttgatgcaggaaaaagcacttacaagacgcggggtgagcttgcctcagtcggggtgagcatgcgtcagcatctcaggaagcagttcttaggaatacaaggatacttacttcttaaggatacagggatacgtgtgatatctacaacttcattgtgttgttgcaacatagaatttgtaggtggacagctactatacgccccaagcatgggttga

SEQ ID NO: 731 - RNA, 245 nt, synthetic construct
  source 1..245: mol_type = other RNA
aaatagttaatagcgccgctgttcatgcttctcggagcctctgaactgtgcaaaatgcgggttagtttggctgttgtcagacagtcttgctttctgaccctggtagctgcccaccccgaagctgctgttccttgtgaacaggatattaggtgcgcccccagtaataagggtgtgggtttaccacagtggtggctactgaatcacctccgagcaaggaggaacccactttaatttttttcgtaaag

SEQ ID NO: 732 - RNA, 318 nt, synthetic construct
  source 1..318: mol_type = other RNA
tatttaatagcgtgcctcgttccccgtgaacaggtaagaagtgtaagggcagtttaattgctttccgccctgtgtagttgtccgcgtctctctaatttgttagagagacacgtcgttaacggagcttgctccgtgaatgacgttccttctgcggaacttttccgtagatatggtgcagggtcgcgcctagcatccaggagctatgtttttataacagtcgtacaacacttctggttataaggatacagctttacgtgtcgtggcagctaccgaatcgcctccgagcaaggaggagtcctccccatctattttttgacg

SEQ ID NO: 733 - RNA, 191 nt, synthetic construct
  source 1..191: mol_type = other RNA
cataatacatgctcttttgagcttctgtataatgctacagtattaacccttttgtagatactgtggaatgggttagtttaacgcttgaaaaagcgtattctttctgaccctagtagctgccaactctacccgtgtggtcatctgattgtttgttagcagtaattgcttgggtaagcagatgctgtttttag

SEQ ID NO: 734 - RNA, 212 nt, synthetic construct
  source 1..212: mol_type = other RNA
ataattctattacgccacggctcatgccagtaatggtctctgtgctgatgctaaacgagttaggttgactattggaagatagtcttgctttctggctctggcgactgtccacctcagaagttgggtgcgttcccagcaaaaaggtgcgggtctaccgcagtggtggttgccgtcttcacctccgagcaaggaggaatctaccctaaaaattt

SEQ ID NO: 735 - RNA, 308 nt, synthetic construct
  source 1..308: mol_type = other RNA
aatgtgattgcgcctcgatagatgctctatgagccgctcggtcgtagaaaaatgggtgagtttgactatctacttcgttagatagtgctgctttccgaccctggcattctgtccgcccttgcagctgcttctcatgggcaagtgaaaacttgctggtgagagggaaaagtcataatttaaagtctcgtctttctagtatgacataggtgcgctctcacgcaatatagggttcagcttttattttatagaagtagagactttcctctagtgacagtgccgaaatgaccccgtgcgaggggtaactacct

SEQ ID NO: 736 - RNA, 202 nt, synthetic construct
  source 1..202: mol_type = other RNA
tattgaacaatagcgtcgcagttcatgctttatgctgatgtgctgcaacaaaaatgggtcaggttgccgctgcacaggcggatttgctttccggccctggttgctacccgtcctgggagattcttccggcgcttaggacattacgggtagttactgaagcgcctccgagcaaggaggaatccttcctattttttggcaaacc

SEQ ID NO: 737 - RNA, 310 nt, synthetic construct
  source 1..310: mol_type = other RNA
gagacaatagcgcagcagttcatgctcatcgagccgctgaactgtgaaaaatgaggggtcagtttgaccgttgtgagacggtcgtgctttccgcccctgaatagctgcccggtcactgactgccatcctggttattgttcatttttgagcaatggatggggatgggaagaaattacattgaaaagagtctcttctccaatgtaacgtaggtgcgcacccagcagaagttgatcctagccttcagcaatgaaggtacagcctagtctgtagcggcagctattaattgcctccgatcaaggaggagtcctcc

SEQ ID NO: 738 - RNA, 275 nt, synthetic construct
  source 1..275: mol_type = other RNA
atgtaaatagcgccgcaacgtattctgcttgcagcgtacgttccgtgaaaaatgagggttagtttggctgtcggcaggcagtcttgctttctgaccctggtagctgctcaccctgatgctgtcaggaaagagacttcggtttctcaactgagattaagtcgtaattgaagagctattctcctttaattatggcgtaggtgcgctcccagcaaaaagggcgcagatatactgctgtagtggctaccgaatcacccccaaccaagggggaacccgct

SEQ ID NO: 739 - RNA, 325 nt, synthetic construct
  source 1..325: mol_type = other RNA
taactaaaagcgtcgcaattcatggcttattaataagtcctctgcatcgccgaaaaatagggttagtttgattgtcggaagatgatttttctttctgaccctggtaagtgcccacttctgaagctgctgtctctagccctcgctaatgtagataggaaagtgccttaatttagatctcgtaactctatattaacggtcaggtgcgctcccagcaataagaagtaagtcttcaagatgaggaacaacttgagactaatccctaagcgggatacagattactggagtggtagttactgaatcacctccttcatcggaggaatccttc

SEQ ID NO: 740 - protein, 7 aa, synthetic construct
PKKKRKV

SEQ ID NO: 741 - protein, 16 aa, synthetic construct
KRPAATKKAGQAKKKK

SEQ ID NO: 742 - protein, 9 aa, synthetic construct
PAAKRVKLD

SEQ ID NO: 743 - protein, 11 aa, synthetic construct
RQRRNELKRSP

SEQ ID NO: 744 - protein, 38 aa, synthetic construct
NQSSNFGPMKGGNFGGRSSGPYGGGGQYFAKPRNQGGY

SEQ ID NO: 745 - protein, 42 aa, synthetic construct
RMRIZFKNKGKDTAELRRRRVEVSVELRKAKKDEQILKRRNV

SEQ ID NO: 746 - protein, 8 aa, synthetic construct
VSRKRPRP

SEQ ID NO: 747 - protein, 8 aa, synthetic construct
PPKKARED

SEQ ID NO: 748 - protein, 8 aa, synthetic construct
PQPKKKPL

SEQ ID NO: 749 - protein, 12 aa, synthetic construct
SALIKKKKKMAP

SEQ ID NO: 750 - protein, 5 aa, synthetic construct
DRLRR

SEQ ID NO: 751 - protein, 7 aa, synthetic construct
PKQKKRK

SEQ ID NO: 752 - protein, 10 aa, synthetic construct
RKLKKKIKKL

SEQ ID NO: 753 - protein, 10 aa, synthetic construct
REKKKFLKRR

SEQ ID NO: 754 - protein, 20 aa, synthetic construct
KRKGDEVDGVDEVAKKKSKK

SEQ ID NO: 755 - protein, 17 aa, synthetic construct
RKCLQAGMNLEARKTKK

SEQ ID NO: 756 - DNA, 42 bp, synthetic construct
  source 1..42: mol_type = other DNA
gctcttccgatctnnnnngatgagcgcattgttagatttcat

SEQ ID NO: 757 - DNA, 20 bp, synthetic construct
  source 1..20: mol_type = other DNA
aaaccgacatcgcaggcttc

SEQ ID NO: 758 - RNA, 16 nt, synthetic construct
  source 1..16: mol_type = other RNA
ctgccgnntaggnagc

(19) United States
(12) Patent Application Publication
(10) Pub. No.: US 2025/0179452 A1
(43) Pub. Date: Jun. 5, 2025

(54) SYSTEMS AND METHODS FOR TRANSPOSING CARGO NUCLEOTIDE SEQUENCES

(71) Applicant: Metagenomi, Inc., Emeryville, CA (US)

(72) Inventors: Brian C. Thomas, Berkeley, CA (US); Christopher Brown, Albany, CA (US); Daniela S.A. Goltsman, Oakland, CA (US); Cristina Noel Butterfield, Oakland, CA (US); Lisa Alexander, Albany, CA (US); Jason Liu, Berkeley, CA (US); Gregory J. Cost, Piedmont, CA (US); Christine Romano, Emeryville, CA (US)

(21) Appl. No.: 18/840,723

(22) PCT Filed: Feb. 23, 2023
(86) PCT No.: PCT/US2023/063181

§ 371 Date: Aug. 22, 2024

Related U.S. Application Data
(60) Provisional application No. 63/313,122, filed on Feb. 23, 2022.

(51) Int. Cl. C12N 9/22; C12N 9/12; C12N 15/11

(52) U.S. Cl. CPC C12N 9/22; C12N 9/1241; C12N 15/111; C12N 2310/20

(57) ABSTRACT

The present disclosure provides systems and methods for transposing a cargo nucleotide sequence into a target nucleic acid site in a target nucleic acid. These systems and methods may comprise a double-stranded nucleic acid comprising the cargo nucleotide sequence, wherein the cargo nucleotide sequence is configured to interact with a recombinase or transposase complex, an effector complex comprising an effector and at least one engineered guide polynucleotide configured to hybridize to the target nucleic acid, and the recombinase or transposase complex wherein said recombinase or transposase complex is configured to recruit the cargo nucleotide to the target nucleic acid site.

### CROSS-REFERENCE

This application is the U.S. National Stage entry of International Application No. PCT/US2023/063181, filed Feb. 23, 2023, which claims the benefit of and priority to U.S. Provisional Patent Application No. 63/313,122, filed Feb. 23, 2022, each of which is hereby incorporated by reference in its entirety for all purposes.

### SEQUENCE LISTING

The contents of the electronic sequence listing (MTG-011WOUS_SL.xml; Size: 942,222 bytes; and Date of Creation: Apr. 4, 2023) is herein incorporated by reference in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaca) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for a system for transposing a cargo nucleotide sequence into a target nucleic acid site comprising: a first double-stranded nucleic acid comprising said cargo nucleotide sequence, wherein said cargo nucleotide sequence is configured to interact with a recombinase or transposase complex; a Cas effector complex comprising a class 2, type II Cas effector and at least one engineered guide polynucleotide configured to hybridize to said target nucleic acid site; and said recombinase or transposase complex, wherein said recombinase or transposase complex is configured to recruit said cargo nucleotide sequence to said target nucleic acid site. In some embodiments, said recombinase or transposase complex binds non-covalently to said Cas effector complex. In some embodiments, said recombinase or transposase complex is covalently linked to said Cas effector complex. In some embodiments, said recombinase or transposase complex is fused to said Cas effector complex in a single polypeptide. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site. In some embodiments, said recombinase or transposase complex is a Tn7 type transposase complex. In some embodiments, said engineered guide polynucleotide is configured to bind said class 2, type II Cas effector. In some embodiments, said class 2, type II Cas effector comprises a polypeptide comprising a sequence having at least 80% identity to SEQ ID NO: 1 or a variant thereof. In some embodiments, said recombinase or transposase complex comprises at least one, at least two, at least three, or four polypeptide(s) comprising a sequence having at least 80% identity to any one of SEQ ID NOs: 2-5 or a variant thereof. In some embodiments, said engineered guide polynucleotide comprises a sequence comprising at least 60-80 consecutive nucleotides having at least 80% identity to SEQ ID NO: 12 or a variant thereof. In some embodiments, said engineered guide polynucleotide comprises a sequence having at least 80% identity to SEQ ID NO: 11 or a variant thereof. In some embodiments, said left-hand recombinase sequence comprises a sequence having at least 80% identity to any one of SEQ ID NOs: 17-18 or a variant thereof. In some embodiments, said right-hand recombinase sequence comprises a sequence having at least 80% identity to SEQ ID NO: 19 or a variant thereof. In some embodiments, said class 2, type II Cas effector and said recombinase or transposase complex are encoded by polynucleotide sequences comprising fewer than about 10 kilobases

In some aspects, the present disclosure provides for a method for transposing a cargo nucleotide sequence into a target nucleic acid site comprising a target nucleotide sequence comprising expressing the system of any of the aspects or embodiments described herein within a cell or introducing the system of any of the aspects or embodiments described herein to a cell.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises a TnsA subunit. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class 2, type V Cas effector is not a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 5′ of said target nucleic acid site. In some embodiments, said engineered guide polynucleotide is configured to bind said class 2, type V Cas effector. In some embodiments, said TnsA subunit comprises a polypeptide having a sequence having at least 80% identity to SEQ ID NO: 7 or a variant thereof. In some embodiments, said Tn7 type transposase complex comprises at least one, at least two, or three polypeptide(s) comprising a sequence having at least 80% identity to any one of SEQ ID NOs: 8-10, or a variant thereof. In some embodiments, said engineered guide polynucleotide comprises a sequence comprising at least about 46-80 consecutive nucleotides having at least 80% identity to any one of SEQ ID NOs: 13-16, or a variant thereof. In some embodiments, said left-hand recombinase sequence comprises a sequence having at least 80% identity to SEQ ID NO: 20, or a variant thereof. In some embodiments, said right-hand recombinase sequence comprises a sequence having at least 80% identity to SEQ ID NO: 21, or a variant thereof. In some embodiments, said class 2, type V Cas effector is not a Cas12k effector. In some embodiments, said class 2, type V Cas effector and said Tn7 type transposase complex are encoded by polynucleotide sequences comprising fewer than about 10 kilobases.

In some aspects, the present disclosure provides for a method for transposing a cargo nucleotide sequence into a target nucleic acid site comprising a target nucleotide sequence comprising expressing the system of any one of any of the aspects or embodiments described herein within a cell or introducing the system of any one of the aspects or embodiments described herein to a cell.

In some aspects, the present disclosure provides for a method for transposing a cargo nucleotide sequence into a target nucleic acid site, comprising contacting a first double-stranded nucleic acid comprising a cargo nucleotide sequence with: a Cas effector complex comprising a class 2, type II Cas effector and at least one engineered guide polynucleotide configured to hybridize to said target nucleic acid site; a recombinase or transposase complex configured to recruit said cargo nucleotide to said target nucleic acid site; and a second double-stranded nucleic acid comprising said target nucleic acid site. In some embodiments, said recombinase or transposase complex binds non-covalently to said Cas effector complex. In some embodiments, said recombinase or transposase complex is covalently linked to said Cas effector complex. In some embodiments, said recombinase or transposase complex is fused to said Cas effector complex in a single polypeptide. In some embodiments, said cargo nucleotide sequence is flanked by a left-hand transposase recognition sequence and a right-hand transposase recognition sequence. In some embodiments, the target nucleic acid further comprises a PAM sequence compatible with said Cas effector complex adjacent to said target nucleic acid site. In some embodiments, said PAM sequence is located 3′ of said target nucleic acid site. In some embodiments, said recombinase or transposase complex is a Tn7 type transposase complex. In some embodiments, said engineered guide polynucleotide is configured to bind said class 2, type II Cas effector. In some embodiments, said class 2, type II Cas effector comprises a polypeptide comprising a sequence having at least 80% identity to SEQ ID NO: 1 or a variant thereof. In some embodiments, said recombinase or transposase complex comprises at least one, at least two, at least three, or four polypeptide(s) comprising a sequence having at least 80% identity to any one of SEQ ID NOs: 2-5 or a variant thereof. In some embodiments, said engineered guide polynucleotide comprises a sequence comprising at least 60-80 consecutive nucleotides having at least 80% identity to SEQ ID NO: 12 or a variant thereof. In some embodiments, said engineered guide polynucleotide comprises a sequence having at least 80% identity to SEQ ID NO: 11 or a variant thereof. In some embodiments, said left-hand recombinase sequence comprises a sequence having at least 80% identity to any one of SEQ ID NOs: 17-18 or a variant thereof. In some embodiments, said right-hand recombinase sequence comprises a sequence having at least 80% identity to SEQ ID NO: 19 or a variant thereof. In some embodiments, said class 2, type II Cas effector and said Tn7 type transposase complex are encoded by polynucleotide sequences comprising fewer than about 10 kilobases.

In some aspects, the present disclosure provides for a method for transposing a cargo nucleotide sequence into a target nucleic acid site, comprising contacting a first double-stranded nucleic acid comprising said cargo nucleotide sequence with: a Cas effector complex comprising a class 2, type V Cas effector and at least one engineered guide polynucleotide configured to hybridize to said target nucleotide sequence; a Tn7 type transposase complex configured to bind said Cas effector complex, wherein said Tn7 type transposase complex comprises a TnsA subunit; and a second double-stranded nucleic acid comprising said target nucleic acid site. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said cargo nucleotide sequence is flanked by a left-hand transposase recognition sequence and a right-hand transposase recognition sequence. In some embodiments, said target nucleic acid site further comprises a PAM sequence compatible with said Cas effector complex adjacent to said target nucleic acid site. In some embodiments, said PAM sequence is located 3′ of said target nucleic acid site. In some embodiments, said engineered guide polynucleotide is configured to bind said class 2, type V Cas effector. In some embodiments, said TnsA subunit comprises a polypeptide having a sequence having at least 80% identity to SEQ ID NO: 7 or a variant thereof. In some embodiments, said Tn7 type transposase complex comprises at least one, at least two, or three polypeptide(s) comprising a sequence having at least 80% identity to any one of SEQ ID NOs: 8-10, or a variant thereof. In some embodiments, said engineered guide polynucleotide comprises a sequence comprising at least about 46-80 consecutive nucleotides having at least 80% identity to any one of SEQ ID NOs: 13-16 or a variant thereof. In some embodiments, said left-hand recombinase sequence comprises a sequence having at least 80% identity to SEQ ID NO: 20, or a variant thereof. In some embodiments, said right-hand recombinase sequence comprises a sequence having at least 80% identity to SEQ ID NO: 21, or a variant thereof. In some embodiments, said class 2, type V Cas effector is not a Cas12k effector. In some embodiments, said class 2, type V Cas effector and said Tn7 type transposase complex are encoded by polynucleotide sequences comprising fewer than about 10 kilobases.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 1, type I-F Cas effector and an engineered guide polynucleotide configured to hybridize to said target nucleotide sequence; and a Tn7 type transposase complex configured to bind said Cas effector complex, wherein said Tn7 type transposase complex comprises a TnsA subunit. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site. In some embodiments, said PAM sequence is located 5′ of said target nucleic acid site. In some embodiments, said engineered guide polynucleotide is configured to bind said class 1, type I-F Cas effector. In some embodiments, said class 1, type I-F Cas effector comprises a polypeptide comprising a sequence having at least 80% identity to any one of SEQ ID NOs: 41-43 and 48-50, or a variant thereof. In some embodiments, said Tn7 type transposase complex comprises at least one, at least two, or three polypeptide(s) comprising a sequence having at least 80% identity to any one of SEQ ID NOs: 44-46, or 51-53, or a variant thereof.

In some aspects, the present disclosure provides for a method for transposing a cargo nucleotide sequence into a target nucleic acid site comprising a target nucleotide sequence comprising expressing the system of any one of the aspects or embodiments described herein within a cell or introducing the system of any one of the aspects or embodiments described herein to a cell.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises TnsB, TnsC, and TniQ components, wherein: (a) said class 2, type V Cas effector comprises a polypeptide having a sequence having at least 80% sequence identity to any one of SEQ ID NOs: 22, 26, 30, 34, 55-89, 104, 147, 264-304, and 660-689, or a variant thereof; or (b) said Tn7 type transposase complex comprises a TnsB, TnsC, or TniQ component having a sequence having at least 80% sequence identity to any one of SEQ ID NOs: 23-25, 27-29, 31-33, 35-37, 101-103, 105-107, 148-150, 305-343, and 345-347, or a variant thereof. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said class 2, type V Cas effector comprises a polypeptide comprising a sequence having at least 80% sequence identity to any one of SEQ ID NOs: 22, 26, 30, 34, 55-89, 104, 147, 264-304, and 660-689, or a variant thereof. In some embodiments, said Tn7 type transposase complex comprises a TnsB, TnsC, or TniQ component comprising a sequence having at least 80% sequence identity to any one of SEQ ID NOs: 23-25, 27-29, 31-33, 35-37, 101-103, 105-107, 148-150, 305-343, and 345-347, or a variant thereof. In some embodiments, said class 2, type V Cas effector is a Cas12k effector. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 5′ of said target nucleic acid site. In some embodiments, said PAM sequence comprises 5′-nGTn-3′ or 5′-nGTt-3′. In some embodiments, said engineered guide polynucleotide is configured to bind said class 2, type V Cas effector. In some embodiments, said TnsB, TnsC, and TniQ components comprise polypeptides having a sequence having at least 80% identity to any one of SEQ ID NOs: 23-25, 27-29, 31-33, 35-37, 101-103, 105-107, 148-150, 305-343, or 345-347, respectively. In some embodiments, said engineered guide polynucleotide comprises a sequence comprising at least about 46-80 consecutive nucleotides having at least 80% identity to any one of SEQ ID NOs: 90, 91, 92, 93, 117, 151, 156-181, or 209-234. In some embodiments, said engineered guide polynucleotide comprises a sequence having at least 80% sequence identity to any one of SEQ ID NOs: 111-114, 201-206, 255, 262, 256, 209, 257, 263, 258, 210, 348, or 350-353, or a variant thereof. In some embodiments, said left-hand recombinase sequence comprises a sequence having at least 80% identity to any one of SEQ ID NOs: 123, 125, 127, 129, 131, 133, 153, 354-358, 461, 463, 465, and 467, or a variant thereof. In some embodiments, said right-hand recombinase sequence comprises a sequence having at least 80% identity to any one of SEQ ID NOs: 124, 126, 128, 130, 132, 154, 155, 359-363, 462, 464, 466, and 468, or a variant thereof. In some embodiments, said class 2, type V Cas effector and said Tn7 type transposase complex are encoded by polynucleotide sequences comprising fewer than about 10 kilobases. In some embodiments: (a) said class 2, type V Cas effector comprises a sequence having at least 80% sequence identity to SEQ ID NO:22 or a variant thereof; (b) said left-hand recombinase sequence comprises a sequence having at least 80% sequence identity to SEQ ID NO:125 or a variant thereof; (c) said right-hand recombinase sequence comprises a sequence having at least 80% identity to SEQ ID NO: 126 or 155, or a variant thereof; (d) said engineered guide polynucleotide: (i) comprises a sequence having at least 80% sequence identity to at least about 46-60 nucleotides of SEQ ID NO: 90; or (ii) comprises a sequence having at least 80% sequence identity to non-degenerate nucleotides of any one of SEQ ID NOs: 94, 112, or 202; or (c) said TnsB, TnsC, and TniQ components comprise sequences having at least 80% sequence identity to any one of SEQ ID NOs: 23-25 or variants thereof. In some embodiments: (a) said class 2, type V Cas effector comprises a sequence having at least 80% sequence identity to SEQ ID NO:26 or a variant thereof; (b) said left-hand recombinase sequence comprises a sequence having at least 80% sequence identity to SEQ ID NO: 127 or a variant thereof; (c) said right-hand recombinase sequence comprises a sequence having at least 880% sequence identity to SEQ ID NO:128 or a variant thereof; (d) said engineered guide polynucleotide: (i) comprises a sequence having at least 80% sequence identity to at least about 46-60 nucleotides of any one of SEQ ID NOs: 91, 156, or 209; or (ii) comprises a sequence having at least 80% sequence identity to non-degenerate nucleotides of any one of SEQ ID NOs: 95, 113, or 203, or (c) said TnsB, TnsC, and TniQ components comprise sequences having at least 80% sequence identity to any one of SEQ ID NOs: 27-29 or variants thereof. In some embodiments: (a) said class 2, type V Cas effector comprises a sequence having at least 80% sequence identity to SEQ ID NO:60 or a variant thereof; (b) said left-hand recombinase sequence comprises a sequence having at least 80% sequence identity to SEQ ID NO:131 or a variant thereof; (c) said right-hand recombinase sequence comprises a sequence having at least 80% sequence identity to SEQ ID NO:132 or a variant thereof; (d) said engineered guide polynucleotide: (i) comprises a sequence having at least 80% sequence identity to at least about 46-60 nucleotides of any one of SEQ ID NOs: 117, 161, or 214; or (ii) comprises a sequence having at least 80% sequence identity to non-degenerate nucleotides of SEQ ID NO: 119; or (c) said TnsB, TnsC, and TniQ components comprise sequences having at least 80% sequence identity to any one of SEQ ID NOs: 101-103 or variants thereof. In some embodiments: (a) said class 2, type V Cas effector comprises a sequence having at least 80% sequence identity to SEQ ID NO: 147 or a variant thereof; (b) said left-hand recombinase sequence comprises a sequence having at least 80% sequence identity to SEQ ID NO:153 or a variant thereof; (c) said right-hand recombinase sequence comprises a sequence having at least 880% sequence identity to SEQ ID NO:154 or a variant thereof; (d) said engineered guide polynucleotide: (i) comprises a sequence having at least 80% sequence identity to at least about 46-60 nucleotides of any one of SEQ ID NOs: 151, 181, or 234; or (ii) comprises a sequence having at least 80% sequence identity to non-degenerate nucleotides of SEQ ID NO: 152 or 254; or (c) said TnsB, TnsC, and TniQ components comprise sequences having at least 80% sequence identity to any one of SEQ ID NOs: 148-150 or variants thereof. In some embodiments: (a) said class 2, type V Cas effector comprises a sequence having at least 80% sequence identity to SEQ ID NO: 34 or a variant thereof; (b) said left-hand recombinase sequence comprises a sequence having at least 80% sequence identity to SEQ ID NO: 129 or a variant thereof; (c) said right-hand recombinase sequence comprises a sequence having at least 880% sequence identity to SEQ ID NO: 130 or a variant thereof; (d) said engineered guide polynucleotide: (i) comprises a sequence having at least 80% sequence identity to at least about 46-60 nucleotides of any one of SEQ ID NOs: 93, 157, or 210; or (ii) comprises a sequence having at least 80% sequence identity to non-degenerate nucleotides of any one of SEQ ID NOs: 97, 114, or 204, or (c) said TnsB, TnsC, and TniQ components comprise sequences having at least 80% sequence identity to any one of SEQ ID NOs: 148-150 or variants thereof. In some embodiments: (a) said class 2, type V Cas effector comprises a sequence having at least 80% sequence identity to SEQ ID NO:30 or a variant thereof; (b) said left-hand recombinase sequence comprises a sequence having at least 80% sequence identity to SEQ ID NO:123 or a variant thereof; (c) said right-hand recombinase sequence comprises a sequence having at least 80% identity to SEQ ID NO: 124, or a variant thereof; (d) said engineered guide polynucleotide: (i) comprises a sequence having at least 80% sequence identity to at least about 46-80 nucleotides of SEQ ID NO:92; or (ii) comprises a sequence having at least 80% identity to the non-degenerate nucleotides of SEQ ID NO:111 or 201; (c) said TnsB, TnsC, and TniQ components comprise polypeptides having a sequence having at least 80% identity to any one of SEQ ID NOs: 31, 32, and 33, or variants thereof; or (f) said PAM sequence comprises 5′-nGTn-3′ or 5′-nGTt-3′.

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises TnsB and TnsC components but does not comprise a TnsA and/or TniQ component. In some embodiments, said transposase complex binds non-covalently to said Cas effector complex. In some embodiments, said transposase complex is covalently linked to said Cas effector complex. In some embodiments, said transposase complex is fused to said Cas effector complex in a single polypeptide. In some embodiments, said Tn7 type transposase complex comprises a polypeptide having a sequencing having at least 80% sequence identity to any one of SEQ ID NOs: 39-40, 109-110, and 344. In some embodiments, said TnsB component comprises a polypeptide comprising a sequence having at least 80% sequence identity to SEQ ID NOs: 40 or 109. In some embodiments, said TnsC component comprises a polypeptide comprising a sequence having at least 80% sequence identity to SEQ ID NOs: 39 or 110. In some embodiments, said class 2, type V Cas effector is a Cas12k effector. In some embodiments, said class 2, type V Cas effector comprises a sequence having at least 80% sequence identity to SEQ ID NO: 38 or SEQ ID NO:108. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, said double-stranded nucleic acid comprising said target nucleic acid site or said system is inside a cell. In some embodiments, the system further comprises a PAM sequence compatible with said Cas effector complex adjacent to said target nucleic acid site. In some embodiments, said PAM sequence is located 5′ of said target nucleic acid site. In some embodiments, said engineered guide polynucleotide is configured to bind said class 2, type V Cas effector. In some embodiments, said TnsB and TnsC components comprise polypeptides having a sequence having at least 80% identity to SEQ ID NOs: 40 and 39 or 109 and 110, respectively. In some embodiments, said engineered guide polynucleotide comprises a sequence comprising at least about 46-80 consecutive nucleotides having at least 80% identity to any one of SEQ ID NOs: 118, 182, 183, 235, and 236, or a variant thereof. In some embodiments, said engineered guide polynucleotide comprises a sequence having at least 80% identity to non-degenerate nucleotides any one of SEQ ID NOs: 115, 116, 205, 206, 261, 235, 260, or 236, or a variant thereof. In some embodiments, said left-hand recombinase sequence comprises a sequence having at least 80% identity to SEQ ID NO:134. In some embodiments, said right-hand recombinase sequence comprises a sequence having at least 80% identity to SEQ ID NO: 135, or a variant thereof. In some embodiments, said class 2, type V Cas effector and said Tn7 type transposase complex are encoded by polynucleotide sequences comprising fewer than about 10 kilobases. In some embodiments (a) said class 2, type V Cas effector comprises a sequence having at least 80% sequence identity to SEQ ID NO: 38 or a variant thereof; (b) said left-hand recombinase sequence comprises a sequence having at least 80% sequence identity to SEQ ID NO:134 or a variant thereof; (c) said right-hand recombinase sequence comprises a sequence having at least 80% identity to SEQ ID NO: 135, or a variant thereof; (d) said engineered guide polynucleotide: (i) comprises a sequence having at least 80% sequence identity to at least about 46-80 nucleotides of SEQ ID NO:182 or 235; or (ii) comprises a sequence having at least 80% identity to the non-degenerate nucleotides of SEQ ID NO: 98, 115-116, 205-206, and 493; or (c) said TnsB and TnsC components comprise polypeptides having a sequence having at least 80% identity to any one of SEQ ID NOs: 40 and 39, or variants thereof.

In some aspects, the present disclosure provides for an engineered nuclease system comprising: an endonuclease comprising a RuvC domain and an HNH domain, wherein said endonuclease is derived from an uncultivated microorganism, wherein said endonuclease is a Class 2, type II endonuclease comprising a sequence having at least 80% identity to SEQ ID NO: 1 or a variant thereof; and an engineered guide polynucleotide, wherein said engineered guide polynucleotide is configured to form a complex with said endonuclease and said engineered guide polynucleotide comprises a spacer sequence configured to hybridize into a target nucleic acid sequence. In some embodiments, said engineered guide polynucleotide comprises at least 60-80 consecutive nucleotides having at least 80% identity to SEQ ID NO:12 or a variant thereof. In some embodiments, said engineered guide polynucleotide comprises a sequence having at least 80% identity to SEQ ID NO: 11 or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class 2, type V endonuclease having at least 80% identity to SEQ ID NO: 5; and an engineered guide polynucleotide, wherein said engineered guide polynucleotide is configured to form a complex with said endonuclease and said engineered guide RNA comprises a spacer sequence configured to hybridize into a target nucleic acid sequence. In some embodiments, said engineered guide polynucleotide comprises a sequence comprising at least about 46-80 consecutive nucleotides having at least 80% identity to any one of SEQ ID NOs: 13-16, or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class 2, type V-K endonuclease having at least 80% identity to any one of SEQ ID NOs: 22, 26, 30, 34, 55-89, 104, 147, 264-304, and 660-689, or a variant thereof; and an engineered guide polynucleotide, wherein said engineered guide polynucleotide is configured to form a complex with said endonuclease and said engineered guide RNA comprises a spacer sequence configured to hybridize into a target nucleic acid sequence. In some embodiments, said engineered guide polynucleotide comprises a sequence comprising at least about 46-80 consecutive nucleotides having at least 80% identity to any one of SEQ ID NOS: 90-93, 117, 151, 156-181, 209-234, 417-460, and 715-739, or a variant thereof. In some embodiments, said engineered guide polynucleotide comprises a sequence having at least 80% sequence identity to non-degenerate nucleotides of any one of SEQ ID NOs: 111-114, 201-206, 255, 262, 256, 209, 257, 263, 258, 210, 348, or 350-353, or a variant thereof.

In some aspects, the present disclosure provides for an engineered nuclease system comprising: an endonuclease comprising a RuvC domain, wherein said endonuclease is derived from an uncultivated microorganism, and wherein said endonuclease is a Class 2, type V-K endonuclease having at least 80% identity to SEQ ID NO: 38 or SEQ ID NO: 108, or a variant thereof; and an engineered guide polynucleotide, wherein said engineered guide polynucleotide is configured to form a complex with said endonuclease and said engineered guide RNA comprises a spacer sequence configured to hybridize into a target nucleic acid sequence. In some embodiments, said engineered guide polynucleotide comprises a sequence comprising at least about 46-80 consecutive nucleotides having at least 80% identity to any one of SEQ ID NOs: 118, 182, 183, 235, and 236, or a variant thereof. In some embodiments, said engineered guide polynucleotide comprises a sequence having at least 80% identity to non-degenerate nucleotides of any one of SEQ ID NOs: 111-114, 201-206, 255, 262, 256, 209, 257, 263, 258, 210, 115, 116, 205, 206, 261, 235, 260, 236, 348, or 350-353, or a variant thereof.

In some aspects, the present disclosure provides for an engineered nuclease system comprising: a Class I, type I-F Cas endonuclease comprising at least one Cas6, Cas7, or Cas8 polypeptide comprising a sequence having at least 80% identity to any one of SEQ ID NOs: 41-43 and 48-50, or a variant thereof; and an engineered guide RNA, wherein said engineered guide RNA is configured to form a complex with said endonuclease and said engineered guide RNA comprises a spacer sequence configured to hybridize into a target nucleic acid sequence. In some embodiments, said engineered guide polynucleotide comprises a sequence having at least 80% identity to non-degenerate nucleotides of any one of SEQ ID NOs: 121, 122, 207, and 208.

In some aspects, the present disclosure provides for a system for transposing a cargo nucleotide sequence into a target nucleic acid site in a target nucleic acid comprising: a Cas effector complex comprising a class 2, type II Cas effector, a small prokaryotic ribosomal protein subunit S15, and an engineered guide polynucleotide configured to hybridize to the target nucleic acid site; a recombinase or transposase complex configured to bind the Cas effector complex; and a double-stranded nucleic acid configured to interact with the recombinase or transposase complex and comprising the cargo nucleotide sequence.

In some embodiments, the Cas effector complex binds non-covalently to the recombinase or transposase complex. In some embodiments, the Cas effector complex is covalently linked to the recombinase or transposase complex. In some embodiments, the Cas effector complex is fused to the recombinase or transposase complex. In some embodiments, the cargo nucleotide sequence is flanked by a left-hand transposase recognition sequence and a right-hand transposase recognition sequence recognized by the recombinase or transposase complex. In some embodiments, the left-hand recombinase sequence comprises a sequence having at least 80% identity to any one of SEQ ID NOs: 17-18. In some embodiments, the right-hand recombinase sequence comprises a sequence having at least 80% identity to SEQ ID NO: 19.

In some embodiments, the target nucleic acid comprises a PAM sequence compatible with the Cas effector complex. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type II Cas effector is not a Cas12k effector. In some embodiments, the class 2, type II Cas effector comprises a polypeptide comprising a sequence having at least 80% identity to SEQ ID NO: 1. In some embodiments, the class 2, type II Cas effector comprises a polypeptide comprising a sequence having at least 90% identity to SEQ ID NO: 1. In some embodiments, the class 2, type II Cas effector comprises a polypeptide comprising a sequence of SEQ ID NO: 1. In some embodiments, the recombinase or transposase complex comprises at least one polypeptide comprising a sequence having at least 80% identity to any one of SEQ ID NOs: 2-5. In some embodiments, the recombinase or transposase complex comprises at least one polypeptide comprising a sequence having at least 90% identity to any one of SEQ ID NOs: 2-5. In some embodiments, the recombinase or transposase complex comprises at least one polypeptide comprising a sequence of any one of SEQ ID NOs: 2-5.

In some embodiments, the engineered guide polynucleotide comprises a sequence comprising at least about 46-80 consecutive nucleotides having at least 80% identity to SEQ ID NO: 12. In some embodiments, the engineered guide polynucleotide comprises a sequence having at least 80% sequence identity to SEQ ID NO: 11.

In some embodiments, the small prokaryotic ribosomal protein subunit S15 comprises a sequence having at least 80% sequence identity to any one of any one of SEQ ID NOs: 494-659. In some embodiments, the class 2, type II Cas effector and the recombinase or transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide configured to hybridize to the target nucleic acid site; a Tn7 type transposase complex configured to bind the Cas effector complex and comprising a TnsA, TnsB, TnsC, and TniQ component; and a double-stranded nucleic acid configured to interact with the Tn7 type transposase complex and comprising the cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex. In some embodiments, the cargo nucleotide sequence is flanked by a left-hand transposase recognition sequence and a right-hand transposase recognition sequence recognized by the recombinase or transposase complex. In some embodiments, the left-hand recombinase sequence comprises a sequence having at least 80% identity to any one of SEQ ID NOs: 20. In some embodiments, the right-hand recombinase sequence comprises a sequence having at least 80% identity to SEQ ID NO: 21.

In some embodiments, the target nucleic acid comprises a PAM sequence compatible with the Cas effector complex. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is not a Cas12k effector. In some embodiments, the TnsA component comprises a polypeptide comprising a sequence having at least 80% identity to SEQ ID NO: 7. In some embodiments, the Tn7 type transposase complex comprises at least one polypeptide comprising a sequence having at least 80% identity to any one of SEQ ID NOs: 8-10.

In some embodiments, the engineered guide polynucleotide comprises a sequence comprising at least about 46-80 consecutive nucleotides having at least 80% identity to any one of SEQ ID NOs: 13-16. In some embodiments, the small prokaryotic ribosomal protein subunit S15 comprises a sequence having at least 80% sequence identity to any one of any one of SEQ ID NOs: 494-659.

In some embodiments, the class 2, type II Cas effector and the recombinase or transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex comprising a class 1, type I-F Cas effector, a small prokaryotic ribosomal protein subunit S15, and an engineered guide polynucleotide configured to hybridize to the target nucleic acid site; a Tn7 type transposase complex configured to bind the Cas effector complex and comprising a TnsA, TnsB, TnsC, and TniQ component; and a double-stranded nucleic acid configured to interact with the Tn7 type transposase complex and comprising a cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex. In some embodiments, the cargo nucleotide sequence is flanked by a left-hand transposase recognition sequence and a right-hand transposase recognition sequence recognized by the recombinase or transposase complex. In some embodiments, the left-hand recombinase sequence comprises a sequence having at least 80% identity to any one of SEQ ID NOs: 136 and 138. In some embodiments, the right-hand recombinase sequence comprises a sequence having at least 80% identity to SEQ ID NO: 137 and 139.

In some embodiments, the target nucleic acid comprises a PAM sequence compatible with the Cas effector complex. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 1, type I-F Cas effector comprises a polypeptide comprising a sequence having at least 80% identity to any one of SEQ ID NOs: 41-43 and 48-50. In some embodiments, the class 1, type I-F Cas effector comprises a polypeptide comprising a sequence having at least 90% identity to any one of SEQ ID NOs: 41-43 and 48-50. In some embodiments, the class 1, type I-F Cas effector comprises a polypeptide comprising a sequence of any one of SEQ ID NOs: 41-43 and 48-50. In some embodiments, the Tn7 type transposase complex comprises at least one polypeptide comprising a sequence having at least 80% identity to any one of SEQ ID NOs: 44-47 and 51-54. In some embodiments, the Tn7 type transposase complex comprises at least one polypeptide comprising a sequence having at least 90% identity to any one of SEQ ID NOs: 44-47 and 51-54. In some embodiments, the Tn7 type transposase complex comprises at least one polypeptide comprising a sequence of any one of SEQ ID NOs: 44-47 and 51-54. In some embodiments, the small prokaryotic ribosomal protein subunit S15 comprises a sequence having at least 80% sequence identity to any one of any one of SEQ ID NOs: 494-659. In some embodiments, the class 2, type II Cas effector and the recombinase or transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to any one of SEQ ID NOs: 22, 26, 30, 34, 55-89, 104, 147, 264-304, and 660-689; and ii) an engineered guide polynucleotide comprising a sequence having at least 80% sequence identity to any one of SEQ ID NOs: 90-93, 111-114, 117, 151, 156-181, 201-204, 209-234, 255-258, 262, 263, 348, 350-353, 417-460, 491-492, and 715-739; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 23-25, 27-29, 31-33, 35-37, 101-103, 105-107, 148-150, 305-343, and 345-347; and a double-stranded nucleic acid configured to interact with the Tn7 type transposase complex and comprising in 5′ to 3′ order: i) a left-hand recombinase sequence comprising a sequence having at least 80% sequence identity to any one of SEQ ID NOs: 123, 125, 127, 129, 131, 133, 153, 354-358, 461, 463, 465, and 467; ii) the cargo nucleotide sequence; and iii) a right-hand recombinase sequence comprising a sequence having at least 80% identity to any one of SEQ ID NOs: 124, 126, 128, 130, 132, 154, 155, 359-363, 462, 464, 466, and 468.

In some aspects, the present disclosure provides for a system for transposing a cargo nucleotide sequence into a target nucleic acid site comprising: a Cas effector complex configured to hybridize to the target nucleic acid site and comprising: i) a class 2, type V Cas effector comprising a polypeptide having a sequence having at least 80% sequence identity to SEQ ID NO: 22; and ii) an engineered guide polynucleotide comprising a sequence having at least 80% sequence identity to any one of SEQ ID NOs: 90, 112, and 202; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 23-25; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 125; ii) the cargo nucleotide sequence; and iii) a right-hand recombinase sequence comprising a sequence having at least 80% identity to any one of SEQ ID NOs: 126 and 155.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to SEQ ID NO: 26; and ii) an engineered guide polynucleotide comprising a sequence having at least 80% sequence identity to any one of SEQ ID NOs: 91, 113, 156, 203, and 209; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 27-29; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 127; ii) the cargo nucleotide sequence; and iii) a right-hand recombinase sequence comprising a sequence having at least 80% identity to SEQ ID NO: 128.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to SEQ ID NO: 60; and ii) an engineered guide polynucleotide comprising a sequence having at least 80% sequence identity to any one of SEQ ID NOs: 117, 119, 161, and 214; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 101-103; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 131; ii) the cargo nucleotide sequence; and iii) a right-hand recombinase sequence comprising a sequence having at least 80% identity to SEQ ID NO: 132.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to SEQ ID NO: 147; and ii) an engineered guide polynucleotide comprising a sequence having at least 80% sequence identity to any one of SEQ ID NOs: 151, 181, and 234; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 148-150; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 153; ii) the cargo nucleotide sequence; and iii) a right-hand recombinase sequence comprising a sequence having at least 80% identity to SEQ ID NO: 154.

In some aspects, the present disclosure provides for a system for transposing a cargo nucleotide sequence into a target nucleic acid site comprising: a Cas effector complex configured to hybridize to the target nucleic acid site in a target nucleic acid and comprising: i) a class 2, type V Cas effector comprising a polypeptide having a sequence having at least 80% sequence identity to SEQ ID NO: 34; and ii) an engineered guide polynucleotide comprising a sequence having at least 80% sequence identity to any one of SEQ ID NOs: 93, 114, 157, 204, and 210; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 148-150; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 129; ii) the cargo nucleotide sequence; and iii) a right-hand recombinase sequence comprising a sequence having at least 80% identity to SEQ ID NO: 130.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to SEQ ID NO: 30; and ii) an engineered guide polynucleotide comprising a sequence having at least 80% sequence identity to any one of SEQ ID NOs: 92, 111, and 201; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 31-33; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 123; ii) the cargo nucleotide sequence; and iii) a right-hand recombinase sequence comprising a sequence having at least 80% identity to SEQ ID NO: 124.

In some aspects, the present disclosure provides for a system for transposing a cargo nucleotide sequence into a target nucleic acid site in a target nucleic acid comprising: a Cas effector complex configured to hybridize to the target nucleic acid site and comprising: i) a class 2, type V Cas effector comprising a polypeptide having a sequence having at least 80% sequence identity to SEQ ID NO: 38; and ii) an engineered guide polynucleotide comprising a sequence having at least 80% sequence identity to any one of SEQ ID NOs: 98, 115-116, 182, 205-206, 235, and 493; a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 39 and 40; and a double-stranded nucleic acid configured to interact with the Tn7 type transposase complex and comprising in 5′ to 3′ order: i) a left-hand recombinase sequence comprising a sequence having at least 80% sequence identity to SEQ ID NO: 134; ii) the cargo nucleotide sequence; and iii) a right-hand recombinase sequence comprising a sequence having at least 80% identity to SEQ ID NO: 135.

In some embodiments, the class 2, type V Cas effector is a Cas12k effector. In some embodiments, the target nucleic acid comprises a PAM sequence compatible with the Cas effector complex. In some embodiments, the PAM sequence is located 5′ of the target nucleic acid site. In some embodiments, the PAM sequence comprises 5′-nGTn-3′ or 5′-nGTt-3′.

In some embodiments, the Cas effector complex further comprises a small prokaryotic ribosomal protein subunit S15. In some embodiments, the small prokaryotic ribosomal protein subunit S15 comprises a sequence having at least 80% sequence identity to any one of SEQ ID NOs: 494-659. In some embodiments, the class 2, type V Cas effector and the Tn7 type transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide configured to hybridize to the target nucleic acid site; a Tn7 type transposase complex configured to bind the Cas effector complex and comprising TnsB and TnsC components but not a TnsA and/or TniQ component; and a double-stranded nucleic acid configured to interact with the Tn7 type transposase complex and comprising the cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex. In some embodiments, the cargo nucleotide sequence is flanked by a left-hand transposase recognition sequence and a right-hand transposase recognition sequence recognized by the recombinase or transposase complex. In some embodiments, the left-hand recombinase sequence comprises a sequence having at least 80% identity to any one of SEQ ID NOs: 134. In some embodiments, the right-hand recombinase sequence comprises a sequence having at least 80% identity to SEQ ID NO: 135.

In some embodiments, the target nucleic acid comprises a PAM sequence compatible with the Cas effector complex. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 80% identity to any one of SEQ ID NOs: 38 and 108. In some embodiments, the class 2, type V Cas effector comprises a polypeptide comprising a sequence having at least 90% identity to any one of SEQ ID NOs: 38 and 108. In some embodiments, the class 2, type V Cas effector comprises a polypeptide comprising a sequence of any one of SEQ ID NOs: 38 and 108. In some embodiments, the TnsB subunit comprises a polypeptide comprising a sequence having at least 80% identity to SEQ ID NOs: 40 or 109. In some embodiments, the TnsC subunit comprises a polypeptide comprising a sequence having at least 80% identity to SEQ ID NOs: 39 or 110. In some embodiments, the Tn7 type transposase complex comprises at least one polypeptide comprising a sequence having at least 80% identity to any one of SEQ ID NOs: 39-40, 109-110, and 344. In some embodiments, the engineered guide polynucleotide comprises a sequence having at least 80% identity to any one of SEQ ID NOs: 115, 116, 205, 206, 261, 235, 260, and 236. In some embodiments, the engineered guide polynucleotide comprises a sequence comprising at least about 46-80 consecutive nucleotides having at least 80% identity to any one of SEQ ID NOs: 118, 182, 183, 235, and 236.

In some embodiments, the small prokaryotic ribosomal protein subunit S15 comprises a sequence having at least 80% sequence identity to any one of SEQ ID NOs: 494-659. In some embodiments, the class 2, type II Cas effector and the recombinase or transposase complex are encoded by polynucleotide sequences comprising fewer than about 10 kilobases.

In some aspects, the present disclosure provides for a system for transposing a cargo nucleotide sequence into a target nucleic acid site in a target nucleic acid comprising: a Cas effector complex comprising a class 2, type II Cas effector, a small prokaryotic ribosomal protein subunit S15, and an engineered guide polynucleotide, the engineered guide polynucleotide capable of hybridizing to the target nucleic acid; a recombinase or transposase complex operably linked to the Cas effector complex; and a double-stranded nucleic acid comprising in 5′ to 3′ order: i) a left-hand recombinase recognition sequence; ii) the cargo nucleotide sequence; and iii) a right-hand recombinase recognition sequence, the left-hand recombinase recognition sequence and the right-hand recombinase recognition sequence capable of being recognized by the recombinase or transposase complex.

In some aspects, the present disclosure provides for a system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide, the engineered guide polynucleotide capable of hybridizing to the target nucleic acid; a Tn7 type transposase complex operably linked to the Cas effector complex and comprising a TnsA, TnsB, TnsC, and TniQ component; and a double-stranded nucleic acid comprising in 5′ to 3′ order: i) a left-hand recombinase recognition sequence; ii) the cargo nucleotide sequence; and iii) a right-hand recombinase recognition sequence, the left-hand recombinase recognition sequence and the right-hand recombinase recognition sequence capable of being recognized by the Tn7 type transposase complex.

In some aspects, the present disclosure provides for a system for transposing a cargo nucleotide sequence into a target nucleic acid site in a target nucleic acid comprising: a Cas effector complex comprising a class 1, type I-F Cas effector, a small prokaryotic ribosomal protein subunit S15, and an engineered guide polynucleotide, the engineered guide polynucleotide capable of hybridizing to the target nucleic acid; a Tn7 type transposase complex operably linked to the Cas effector complex and comprising a TnsA, TnsB, TnsC, and TniQ component; and a double-stranded nucleic acid comprising in 5′ to 3′ order: i) a left-hand recombinase recognition sequence; ii) the cargo nucleotide sequence; and iii) a right-hand recombinase recognition sequence, the left-hand recombinase recognition sequence and the right-hand recombinase recognition sequence capable of being recognized by the Tn7 type transposase complex.

In some aspects, the present disclosure provides for an engineered nuclease system comprising: an endonuclease comprising a RuvC domain and an HNH domain, wherein the endonuclease is derived from an uncultivated microorganism, wherein the endonuclease is a Class 2, type II endonuclease comprising a sequence having at least 80% identity to SEQ ID NO: 1; and an engineered guide polynucleotide, wherein the engineered guide RNA is configured to form a complex with the endonuclease and the engineered guide RNA comprises a spacer sequence configured to hybridize into a target nucleic acid sequence. In some embodiments, the engineered guide polynucleotide comprises at least 60-80 consecutive nucleotides having at least 80% identity to SEQ ID NO: 12. In some embodiments, the engineered guide polynucleotide comprises a sequence having at least 80% identity to SEQ ID NO: 11.

In some aspects, the present disclosure provides for an engineered nuclease system comprising: an endonuclease comprising a RuvC domain, wherein the endonuclease is derived from an uncultivated microorganism, and wherein the endonuclease is a Class 2, type V endonuclease having at least 80% identity to SEQ ID NO: 6; and an engineered guide RNA, wherein the engineered guide RNA is configured to form a complex with the endonuclease and the engineered guide RNA comprises a spacer sequence configured to hybridize into a target nucleic acid sequence. In some embodiments, the engineered guide polynucleotide comprises a sequence comprising at least about 46-80 consecutive nucleotides having at least 80% identity to any one of SEQ ID NOS: 13-16.

In some aspects, the present disclosure provides for an engineered nuclease system comprising: an endonuclease comprising a RuvC domain, wherein the endonuclease is derived from an uncultivated microorganism, and wherein the endonuclease is a Class 2, type V-K endonuclease having at least 80% identity to any one of SEQ ID NOs: 22, 26, 30, 34, 55-89, 104, 147, 264-304, and 660-689; and an engineered guide RNA, wherein the engineered guide RNA is configured to form a complex with the endonuclease and the engineered guide RNA comprises a spacer sequence configured to hybridize into a target nucleic acid sequence. In some embodiments, the engineered guide polynucleotide comprises a sequence comprising at least about 46-80 consecutive nucleotides having at least 80% identity to any one of SEQ ID NOs: 90-93, 117, 151, 156-181, 209-234, 417-460, and 715-739. In some embodiments, the engineered guide polynucleotide comprises a sequence having at least 80% sequence identity to any one of SEQ ID NOs: 111-114, 201-206, 209, 210, 255-258, 262, 263, 348, 350-353, and 473-492.

In some aspects, the present disclosure provides for an engineered nuclease system comprising: an endonuclease comprising a RuvC domain, wherein the endonuclease is derived from an uncultivated microorganism, and wherein the endonuclease is a Class 2, type V-K endonuclease having at least 80% identity to SEQ ID NO: 38 or SEQ ID NO: 108; and an engineered guide RNA, wherein the engineered guide RNA is configured to form a complex with the endonuclease and the engineered guide RNA comprises a spacer sequence configured to hybridize into a target nucleic acid sequence. In some embodiments, the engineered guide polynucleotide comprises a sequence comprising at least about 46-80 consecutive nucleotides having at least 80% identity to any one of SEQ ID NOs: 118, 182, 183, 235, and 236. In some embodiments, the engineered guide polynucleotide comprises a sequence having at least 80% identity to any one of SEQ ID NOs: 111-114, 115, 116, 201-206, 209, 210, 235, 236, 255-258, 260-263, 348, and 350-353.

In some aspects, the present disclosure provides for an engineered nuclease system comprising: a Class 1, type I-F Cas endonuclease comprising at least one Cas6, Cas7, or Cas8 polypeptide comprising a sequence having at least 80% identity to any one of SEQ ID NOs: 41-43 and 48-50; and an engineered guide RNA, wherein the engineered guide RNA is configured to form a complex with the endonuclease and the engineered guide RNA comprises a spacer sequence configured to hybridize into a target nucleic acid sequence. In some embodiments, the engineered guide polynucleotide comprises a sequence having at least 80% identity to any one of SEQ ID NOS: 121, 122, 207, and 208.

In some aspects, the present disclosure provides for a method for transposing a cargo nucleotide sequence into a target nucleic acid site comprising introducing a system of the disclosure to a cell.

In some aspects, the present disclosure provides for a cell comprising a system of the disclosure. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HcLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts example organizations of CRISPR/Cas loci of different classes and types.

FIG. 2 depicts the architecture of a natural Class 2 Type II crRNA/tracrRNA pair, compared to a hybrid sgRNA wherein the crRNA and tracrRNA are joined.

FIG. 3 depicts the two pathways found in Tn7 and Tn7-like elements.

FIGS. 4A-4C depict the genomic context of a Type II Tn7 reduced CAST of the family MG36. FIG. 4A shows the MG36-5 CAST system comprises a CRISPR array (CRISPR repeats), a Type II nuclease with RuvC and HNH endonuclease domains, and four predicted transposase protein open reading frames. The catalytic transposase TnsB is encoded as two subunits. FIG. 4B shows two transposon ends are predicted for the MG36-1 CAST system (TIR-1 and TIR-2). FIG. 4C shows alignment of the predicted Type II Tn7 reduced CAST transposon left end (LE) and right end (RE) sequences, with annotated repeats as arrows. The left and right ends were labeled by their orientation.

FIGS. 5A-5B depict the genomic context of a Type V Tn7 CAST of the family MG39. FIG. 5A shows the MG39-1 CAST system consists of a Type V nuclease, four predicted transposon proteins (TnsABC and TniQ), and a CRISPR array. The transposon ends were predicted for the MG39-1 CAST system (TIR-1). FIG. 5B shows the alignment of the predicted Type V Tn7 CAST transposon left end (LE) and right end (RE) sequences, with annotated inverted repeats represented as arrows.

FIG. 6 and FIG. 7 depict predicted structures (predicted, for example in Example 3) of corresponding sgRNAs of CAST systems described herein.

FIG. 8 depicts the genomic context of MG108-1, a system described herein. This candidate is a Cas12K CAST which naturally lacks TniQ. Genes in the genomic fragment are represented by arrows.

FIG. 9 depicts the phylogenetic gene tree of Cas12k effector sequences. The tree was inferred from a multiple sequence alignment of 64 Cas12k sequences recovered here (orange and black branches) and 229 reference Cas12k sequences from public databases (grey branches). Orange branches indicate Cas12k effectors with confirmed association with CAST transposon components.

FIGS. 10A-10C depict MG110 Cascade CAST. FIG. 10A shows genomic context of the MG110-1 Cascade CAST. Full Tn7 suite (TnsA, TnsB, TnsC/TniB, TniQ) and defective Cascade suite (Cas6, Cas7, fused Cas5-Cas8) are represented by orange arrows. TIR flanking the CAST transposon are represented by connected arrows. FIG. 10B shows repeat secondary structure indicates a stem-loop structure of the crRNA. FIG. 10C shows sequence alignment of CRISPR repeats from A. wodanis, V. cholerae, and the MG110 family CASTs indicates conserved motifs indicative of the crRNA stem-loop secondary structure.

FIG. 11A depicts the MG64-3 CRISPR locus. The tracrRNA is encoded upstream from the CRISPR array, while the transposon end is encoded downstream (inner black box). A sequence corresponding to a partial 3′ CRISPR repeat and a partial spacer are encoded within the transposon (outer box). The self-matching spacer is encoded outside of the transposon end.

FIG. 11B depicts tracrRNA sequence alignment for various CASTs provided herein. Alignment of tracrRNA sequences shows regions of conservation. In particular, the sequence “TGCTTTC” at sequence position 92-98 (top box) may be important for sgRNA tertiary structure and for a non-continuous repeat-anti-repeat pairing with the crRNA. The hairpin “CYCC (n6) GGRG” at positions 265-278 (bottom box) may be important for function, such as by positioning the downstream sequence for crRNA pairing.

FIG. 11C shows presence of repeat-anti-repeat (RAR) motifs in e.g., MG64-2, MG64-4, MG64-5, MG64-6, MG64-7, and MG108-1 families.

FIG. 12A depicts the predicted structure of MG64-2 sgRNA.

FIG. 12B depicts the predicted structure of MG64-4 sgRNA.

FIG. 12C depicts the predicted structure of MG64-6 sgRNA.

FIG. 12D depicts the predicted structure of MG64-7 sgRNA.

FIG. 12E depicts the predicted structure of MG108-1 sgRNA.

FIGS. 13A-13C depict PCR, PAM, and Sanger sequencing data which demonstrate that MG64-6 is active in vitro. Using the protocol described for In vitro targeted integrase activity, the effector protein and its TnsB, TnsC, and TniQ proteins were expressed in an in vitro transcription/translation system. After translation, the target DNA, cargo DNA, and sgRNA were added in reaction buffer. Integration was assayed by PCR across the target/donor junctions. FIG. 13A depicts a gel image of PCRs of transposition showing apo (no sgRNA) and 64-6 with sgRNA 64-6 sgRNA. The PCR 3 detects the RE junction, PAM distal. PCR 4 is LE junction, PAM distal. PCR 5 is RE junction, PAM proximal. PCR 6 is LE junction, PAM proximal. The PCRs are paired across the different possible orientations (PCR 3 and 6 vs PCR 4 and 5). The LE-PAM proximal and RE-PAM distal orientation is preferred. FIG. 13B depicts PAMs from the in vitro transposition assay, sequencing PCRs 5 and 6. FIG. 13C depicts Sanger data which shows the junction of transposition where the excision occurs in the donor DNA. The first panel shows PCRs 3 and 5 (the RE). The second panel shows PCR 4 and 6 (the LE). The Sanger sequencing reaction is of the donor-target product, so the point where the sequencing stops matching the donor DNA is when junction occurs (dark bars underneath sequencing peaks)

FIG. 14 depicts next-generation sequencing (NGS) results of the in vitro transposition products which reveal the insertion site preferences. The NGS reads were processed in CRISPResso2 compared to a reference sequence with transposition at position 60. Indels from this correspond to transpositions earlier or later than this arbitrary reference sequence.

FIG. 15 depicts electrophoretic mobility shift assay (EMSA) results of the 64-2 TnsB and its RE DNA sequence. The EMSA results confirm binding and TnsB recognition. The TnsB protein was expressed in an in vitro transcription/translation system, incubated with FAM-labeled DNA containing the RE sequence, and then separated on a native 5% TBE gel. Binding is observed as a shift upwards in the labeled band. Multiple TnsB binding sites leads to multiple shifts in the EMSA. Lane 1: FAM-labeled DNA only. Lane 2: FAM DNA plus the in vitro transcription/translation system (no TnsB protein). Lane 3: FAM DNA plus TnsB. Upshift of the labeled band in Lane 3 indicates binding of the RE sequence by TnsB, indicating it contains an active RE transposition sequence.

FIGS. 16A-16B depict Cas12k effector diversity. FIG. 16A depicts Cas12k CAST genomic context. The transposon is characterized by terminal inverted repeats (TIR, light orange bars), Tn7-like transposon genes (colored arrows), the dead effector Cas12k (orange arrow), a tracrRNA (pink half arrow), and CRISPR array. A “TAAA” target site duplication (TDS) was observed flanking the TIRs. Middle panel: MG64-1 non-coding region inset showing the tracrRNA, a pseudo repeat and self-targeting spacer, the CRISPR array and transposon left end TIR. Bottom panel: multiple alignment of the pseudo repeat and self-targeting spacer in a group of CAST homologs. FIG. 16B depicts unrooted phylogenetic tree of Cas12k effectors. Cas12k effectors as described here are shown as orange (confirmed transposon in the genome) and black branches, while reference Cas12k sequences are shown in grey. Reference sequences ShCas12k and AcCas12k are shown with red arrows.

FIGS. 17A-17B depicts multiple sequence alignment of CAST right (FIG. 17A) and left (FIG. 17B) ends. Transposon ends inverted motif “TGTNNA” is highlighted with a box.

FIG. 18 depicts alignment of Cas12k CAST tracrRNA sequences, showing regions of sequence and structural conservation. In particular, the sequence “TGCTTTC” at sequence position 90 (top box) may be important for sgRNA tertiary structure and for a non-continuous repeat-anti-repeat pairing with the crRNA. The hairpin “CYCC (n6) GGRG” at position 331 (bottom box) may be important for function, such as by positioning the downstream sequence for crRNA pairing.

FIG. 19 depicts single guide RNA folding of an active MG64-6 CAST system.

FIGS. 20A-20B depict in vitro screening of CAST transposition with a PAM library. FIG. 20A depicts the screening setup of in vitro PAM determination. FIG. 20B depicts a schematic of junction PCR for the detection of transposition products.

FIG. 21A depicts transposition junctions of MG64-6 CAST amplified by PCR.

FIG. 21B depicts SeqLogo representation of detected PAMs for MG64-6.

FIG. 21C depicts integration frequency plotted by distance on proximal and distal distances of MG64-6.

FIGS. 22A-22C depict the results of E. coli integration with MG64-6. FIG. 22A depicts a schematic representation of introduction of a CAST system into E. coli. FIG. 22B depicts NGS data showing greater than 80% editing efficiency. FIG. 22C depicts off-target analysis showing that off-target integration greater than 1% of all the summed transposition events was not detected.

FIGS. 23A-23B depict insertion rates into various endogenous loci of the E. coli genome. FIG. 23A depicts local insertion rates for various endogenous loci of the E. coli genome. FIG. 23B depicts the off-target rate for insertion into various endogenous loci of the E. coli genome.

FIG. 24 depicts NLS Screening of MG64-6 CAST components. All CAST components were synthesized with NLS tags on both N and C termini and expressed in vitro. All components were then tested in in-vitro transposition reactions with MG64-6 donor PCR fragment, single guide RNA, and a target plasmid. Row A: Lane 1=all WT 64-6 CAST components without any NLS tags in apo conditions (without single guide). Lane 2=all WT 64-6 CAST components without any NLS tags in holo condition (with single guide). Lane 3=in vitro transposition with NLS-MG64-6-Cas12k, Lane 4=in vitro transposition with MG64-6-Cas12k-NLS, Lane 5=NLS-MG64-6-B, Lane 6=MG64-6-B-NLS, Lane 7=NLS-MG64-6-C Lane 8=MG64-6-C-NLS, Lane 9=NLS MG64-6-Q Lane 10=MG64-6-NLS. Row B: Combinatorial testing of the CAST components to find active sets proteins in vitro. All reactions are holo conditions (with single guide) except for Lane 2. Lane 1=all WT 64-6 CAST components without any NLS tags in apo conditions (without single guide). Lane 2=all WT 64-6 CAST components without any NLS tags in holo condition (with single guide) Lane 3=NLS-MG64-6-Cas12k, NLS-MG64-6-B, NLS-MG64-6-C, NLS-MG64-6-Q, Lane 4=NLS-MG64-6-Cas12k, NLS-MG64-6-B, NLS-MG64-6-C, MG64-6-Q-NLS, Lane 5=NLS-MG64-6-Cas12k, MG64-6-B-NLS, NLS-MG64-6-C, NLS-MG64-6-Q, Lane 6=NLS-MG64-6-Cas12k, MG64-6-B-NLS, NLS-MG64-6-C, MG64-6-Q-NLS, Lane 7=MG64-6-Cas12k-NLS, NLS-MG64-6-B, NLS-MG64-6-C, NLS-MG64-6-Q, Lane 8=MG64-6-Cas12k-NLS, NLS-MG64-6-B, NLS-MG64-6-C, MG64-6-Q-NLS, Lane 9=MG64-6-Cas12k-NLS, MG64-6-B-NLS, NLS-MG64-6-C, NLS-MG64-6-Q, Lane 10=MG64-6-Cas12k-NLS, MG64-6-B-NLS, NLS-MG64-6-C, MG64-6-Q-NLS

FIG. 25 depicts gel images of PCR junction of in vitro transposition reactions with in vitro expressed CAST components. Cell derived materials were extracted from lentiviral transduced cell lines with expressed CAST NLS components. For each cell extraction, both cytoplasmic and nuclear fractions were tested with a complement set of WT CAST components. Boxed lanes are not relevant for this experiment. Row A: Lane 1=all in vitro expressed CAST components apo condition (no single guide added), Lane 2=all in vitro expressed CAST components holo conditions (single guide added), Lane 3=Cytoplasmic NLS-MG64-6-Cas12k, Lane 4=Cytoplasmic MG64-6-Cas12k-NLS, Lane 5=Cytoplasmic NLS-MG64-6-B, Lane 6=Cytoplasmic MG64-6-B-NLS, Lane 7=Cytoplasmic NLS-MG64-6-C, Lane 8=Cytoplasmic NLS-MG64-6-Q. Row B: Lane 1=Cytoplasmic MG64-6-Q-NLS, Lane 2=Nucleoplasmic NLS-MG64-6-Cas12k, Lane 3=Nucleoplasmic MG64-6-Cas12k-NLS, Lane 4=Nucleoplasmic NLS-MG64-6-B, Lane 5=Nucleoplasmic MG64-6-B-NLS, Lane 6=Nucleoplasmic NLS-MG64-6-C, Lance 7=Nucleoplasmic NLS-MG64-6-Q, Lane 8=Nucleoplasmic MG64-6-Q-NLS. Row C: Lane 1=all in vitro expressed CAST components apo condition (no single guide added), Lane 2=all in vitro expressed CAST components holo conditions (single guide added), Lane 3=skip, Lance 4=skip, Lane 5=Cytoplasmic polycistronic NLS-MG64-6-B and NLS-MG64-6-C, Lane 6=Cytoplasmic polycistronic MG64-6-B-NLS and NLS-MG64-6-C, Lane 7=skip, Lane 8=skip, Lane 9=Nucleoplasmic polycistronic NLS-MG64-6-B and NLS-MG64-6-C, Lane 6=Nucleoplasmic polycistronic MG64-6-B-NLS and NLS-MG64-6-C

FIGS. 26A-26B depict Sanger sequencing data of the integration PCR product which demonstrates that MG64-6 is active in vitro. The reaction is of the donor-target product and the point where the sequencing stops matching the donor DNA is when junction occurs (dark bars underneath sequencing peaks). FIG. 26A depicts Sanger sequencing data for PCR reactions 3 and 5 (RE). FIG. 26B depicts Sanger sequencing data for PCR reactions 4 and 6 (LE).

FIGS. 27A-27C illustrate in vitro screening of MG64-6 Cas12k CAST transposition with homologous Cas12k sgRNAs and effectors. FIG. 27A depicts a schematic illustration of junction PCR for the detection of transposition products. A target substrate with a 5′ PAM followed by the protospacer (Target, Rxn #1) is targeted with the CAST system to integrate cargo DNA (Rxn #2). Upon successful integration, junction PCR reactions are performed with primers to amplify the four putative integration reactions, based on the orientation of cargo integration. FIG. 27B depicts junction PCR reactions for transposition activity of MG64-6 with homologous sgRNAs. Left gel: Rxn #3. Lane 4: ladder. Lanes 1-3: transposition reactions with sgRNA from effectors MG64-57, MG108-1, and MG108-2. Right gel: Lane 10: ladder. Lanes 1-3: Rxn #5 from transposition reactions with sgRNA from effectors MG64-57, MG108-1, and MG108-2. Lanes 7-9: Rxn #6 from transposition reactions with sgRNA from effectors MG64-57, MG108-1, and MG108-2. Boxed lanes are not relevant for this experiment. FIG. 27C depicts junction PCR reactions for transposition activity of MG64-6 with homologous Cas12k effectors. Lane 13: ladder. Lanes 1-12: Rxn #5 from transposition reactions with the Cas12k effector MG64-57 and MG64-6 transposition proteins. 6Tns: MG64-6 TnsB, TnsC and TniQ. 6B: MG64-6 TnsB. 6C: MG64-6 TnsC. 6Q: MG64-6 TniQ.

FIG. 28 depicts results of immunofluorescence staining for localization of Cas12k CAST components in human cells. All rows: CAST proteins were tagged with an HA tag (Cas12k and TnsB) or FLAG tag (TnsC and TniQ). Anti-HA or Anti-FLAG antibody was used for protein detection. DAPI is used to stain DNA (nucleus, First row). Merged DAPI and Anti-tag channels indicate protein localization (row 2). MG64-6 Cas12k, TnsB and TniQ localize in the nucleus, while TnsC localizes both in the nucleus and in the cytoplasm (row 3).

FIGS. 29A and 29B depict the design and testing of engineered minimal LE and RE of MG64-6. FIG. 29A depicts a schematic illustration of inverted repeats across the WT 64-6 Terminal Inverted Repeats (TIR) and minimal LE/RE designed. FIG. 29B depicts junction PCR results of RE1 to PAM target (Min) vs. the wild type RE (WT). A shift in PCR amplified band size represents is expected for a smaller sized RE in the final transposition fragment.

FIG. 30 depicts a schematic illustration of the identification of ribosomal protein S15 homologs in Cyanobacterial genomic fragments. Candidate sequences from the same samples from where Cas12k effectors were recovered are highlighted by dark dots. The reference S15 from E. coli is shown by an arrow.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG36

SEQ ID NO: 1 shows a full-length peptide sequence of a MG36 Cas effector.

SEQ ID NOs: 2-5 show peptide sequences of MG36 transposition proteins that may comprise a recombinase or transposase complex associated with a MG36 Cas effector. The addition of -B1, -B2, -T1, and -C to the end of the labels denotes similarity to TnsB1, TnsB2, TnsT1, and TniC proteins of Tn7-like systems, respectively.

SEQ ID NO: 11 shows a nucleotide sequence of an sgRNA engineered to function with an MG36 Cas effector.

SEQ ID NO: 12 shows a nucleotide sequence of a MG36 tracrRNAs derived from the same loci as a MG36 Cas effector.

SEQ ID NOs: 17-18 show nucleotide sequences of left-hand transposase recognition sequences associated with a MG36 system.

SEQ ID NO: 19 shows a nucleotide sequence of a right-hand transposase recognition sequence associated with a MG36 system.

### MG39

SEQ ID NO: 6 shows the full-length peptide sequence of a MG39-1 Cas effector.

SEQ ID Nos: 7-10 show the peptide sequences of MG39-1 transposition proteins that may comprise a recombinase or transposase complex associated with the MG39-1 Cas effector.

SEQ ID NOs: 13-16 show nucleotide sequences of MG39 tracrRNAs derived from the same loci as a MG39 Cas effector.

SEQ ID NO: 20 shows a nucleotide sequence of a left-hand transposase recognition sequence associated with a MG39 system.

SEQ ID NO: 21 shows a nucleotide sequence of a right-hand transposase recognition sequence associated with a MG39 system.

### MG64

SEQ ID NOs: 22, 26, 30, 34, 55-89, 104, 147, 264-304, and 660-689 show the full-length peptide sequences of MG64 Cas effectors.

SEQ ID NOs: 23-25, 27-29, 31-33, 35-37, 101-103, 105-107, 148-150, 305-343, and 345-347 show the peptide sequences of MG64 transposition proteins that may comprise a recombinase or transposase complex associated with MG64 Cas effectors. The addition of -A, -B, -C, and -Q to the end of the labels denotes similarity to TnsA, TnsB, TnsC, and TniQ proteins of Tn7-like systems, respectively.

SEQ ID NOs: 90-93, 117, 151, 156-181, 209-234, 417-460, and 715-739 show nucleotide sequences of MG64 tracrRNAs derived from the same loci as a MG64 effector.

SEQ ID NOs: 94-97, 119, 152, and 184-200 show nucleotide sequences of MG64 target CRISPR repeats.

SEQ ID NOs: 237-259, 364-416, and 690-714 show nucleotide sequences of MG64 crRNAs.

SEQ ID NOs: 111-114, 201-204, 262, 263, 348, 350-353, and 473-492 show nucleotide sequences of single guide RNAs engineered to function with MG64 Cas effectors.

SEQ ID NOs: 123, 125, 127, 129, 131, 133, 153, 354-358, 461, 463, 465, and 467 show nucleotide sequences of left-hand transposase recognition sequences associated with a MG64 system.

SEQ ID NOs: 124, 126, 128, 130, 132, 154, 155, 359-363, 462, 464, 466, and 468 show nucleotide sequences of right-hand transposase recognition sequences associated with a MG64 system.

### MG108

SEQ ID NOs: 38, and 108 show the full-length peptide sequences of MG108 Cas effectors.

SEQ ID NOs: 39-40, 109-110, and 344 show the peptide sequences of MG108 transposition proteins that may comprise a recombinase or transposase complex associated with MG108 Cas effectors. The addition of -A, -B, -C, and -Q to the end of the labels denotes similarity to TnsA, TnsB, TnsC, and TniQ proteins of Tn7-like systems, respectively.

SEQ ID NO: 98 and 120 show nucleotide sequences of MG108 target CRISPR repeats.

SEQ ID NO: 260-261 show nucleotide sequences of MG108 crRNAs.

SEQ ID NOs: 115-116, 205-206, and 493 show nucleotide sequences of single guide RNAs engineered to function with MG108 Cas effectors.

SEQ ID NOs: 118, 182-183, and 235-236 show nucleotide sequences of MG108 tracrRNAs derived from the same loci as a MG108 effector.

SEQ ID NO: 134 shows a nucleotide sequence of a left-hand transposase recognition sequence associated with a MG108 system.

SEQ ID NO: 135 shows a nucleotide sequence of a right-hand transposase recognition sequence associated with a MG108 system.

### MG110

SEQ ID NOs: 41-43 and 48-50 show the full-length peptide sequences of MG110 Cas effectors. The addition of -6, -7, and -8 to the end of the labels denotes similarity to cas6, cas7, and cas8 proteins of class I, type I-F systems, respectively.

SEQ ID NOs: 44-47 and 51-54 show the peptide sequences of MG110 transposition proteins that may comprise a recombinase or transposase complex associated with MG110 Cas effectors. The addition of -A, -B, -C, and -Q to the end of the labels denotes similarity to TnsA, TnsB, TnsC, and TniQ proteins of Tn7-like systems, respectively.

SEQ ID NOs: 99-100 show nucleotide sequences of MG110 target CRISPR repeats.

SEQ ID NOs: 121-122 and 207-208 show nucleotide sequences of MG110 crRNAs.

SEQ ID NOs: 136 and 138 show nucleotide sequences of left-hand transposase recognition sequences associated with a MG110 system.

SEQ ID NOs: 137 and 139 show nucleotide sequences of right-hand transposase recognition sequences associated with a MG110 system.

### MG190

SEQ ID Nos: 494-659 show peptide sequences of MG190 ribosomal proteins.

### Other Sequences

SEQ ID NOs: 140-141, 471-472, and 740-755 show peptide sequences of nuclear localizing signals.

SEQ ID NOs: 142-143 and 470 show peptide sequences of linkers.

SEQ ID NOs: 144-146 show peptide sequences of epitope tags.

SEQ ID NO: 469 shows the peptide sequence of an E. coli promoter.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of didcoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodaminc (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Bochringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. In a polynucleotide when referring to a T, a T means U (Uracil) in RNA and T (Thymine) in DNA. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” refer to introduction of a nucleic acid into a cell by non-viral or viral-based methods. The nucleic acid molecules may be gene sequences encoding complete proteins or functional portions thereof. Sec, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may refer to a promoter that contains all the basic necessary elements to promote transcriptional expression of an operably linked polynucleotide. Eukaryotic basal promoters typically, though not necessarily, contain a TATA-box and/or a CAAT box. In some embodiments, different promoters direct the expression of a gene in different tissues or cell types, or at different stages of development, or in response to different environmental or physiological conditions or inducer molecules. Promoters that cause a gene to be expressed in most cell types most of the time are commonly referred to as “constitutive promoters.” Promoters that cause the expression of genes in a particular cell and tissue type are commonly referred to as “cell-specific promoters” or “tissue-specific promoters,” respectively. Promoters that cause the expression of genes at specific stages of development or cell differentiation are commonly referred to as “development-specific promoters” or “cell differentiation-specific promoters.” Promoters that induce and result in the expression of genes after exposing or treating cells with agents, biomolecules, chemicals, ligands, light, etc. that induce the promoters are commonly referred to as “inducible promoters” or “regulatable promoters.” It is further recognized, in some embodiments, that since the exact boundaries of regulatory sequences have not been completely defined in most cases, DNA fragments of different lengths have the same promoter activity.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

The terms “engineered,” “synthetic,” and “artificial” are used interchangeably herein to refer to an object that has been modified by human intervention. For example, the terms may refer to a polynucleotide or polypeptide that is non-naturally occurring. An engineered peptide may have, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. For example, VPR and VP64 domains are synthetic transactivation domains. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

The term “tracrRNA” or “tracr sequence,” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid . . . . If the tracrRNA is engineered, it may have about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes, S. aureus, etc. or SEQ ID NOs: *_*). tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA (e.g., a tracrRNA from S. pyogenes, S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” or “guide polynucleotide” refers to a nucleic acid that may hybridize to a target nucleic acid and thereby directs an associated nuclease to the target nucleic acid. A guide nucleic acid may be RNA (guideRNA or gRNA). A guide nucleic acid may be DNA. A guide nucleic acid may be a mixture of RNA and DNA. A guide nucleic acid may comprise a crRNA or a tracrRNA or a combination of both. A guide nucleic acid may be engineered. The guide nucleic acid may be programmed to specifically bind to the target nucleic acid. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment,” a “nucleic acid-targeting sequence,” or a “spacer.” A nucleic acid-targeting segment may comprise a sub-segment that may be referred to as a “protein binding segment” or “protein binding sequence” or “Cas protein binding segment.”

As used herein, the terms “gene editing” and “genome editing” can be used interchangeably. Gene editing or genome editing means to change the nucleic acid sequence of a gene or a genome. Genome editing can include, for example, insertions, deletions, and mutations.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and I to extend gaps for sequences of less than 30 residues (these are the default parameters for BLASTP in the BLAST suite available at https://blast.ncbi.nlm.nih.gov); CLUSTALW with parameters of; the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

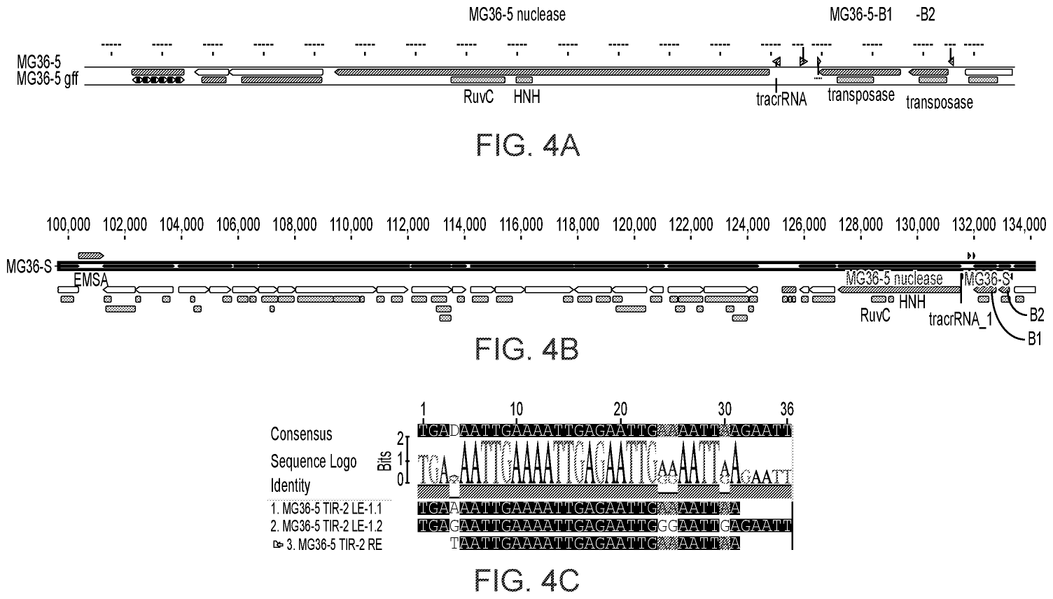

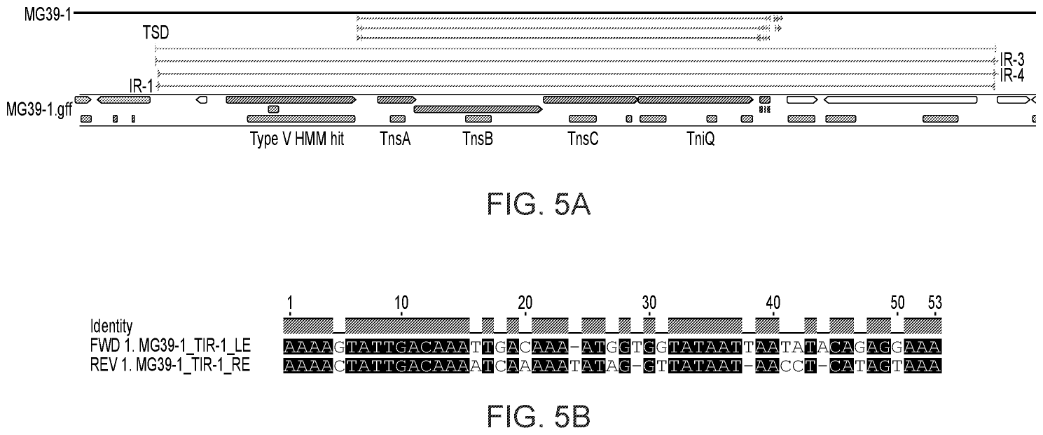

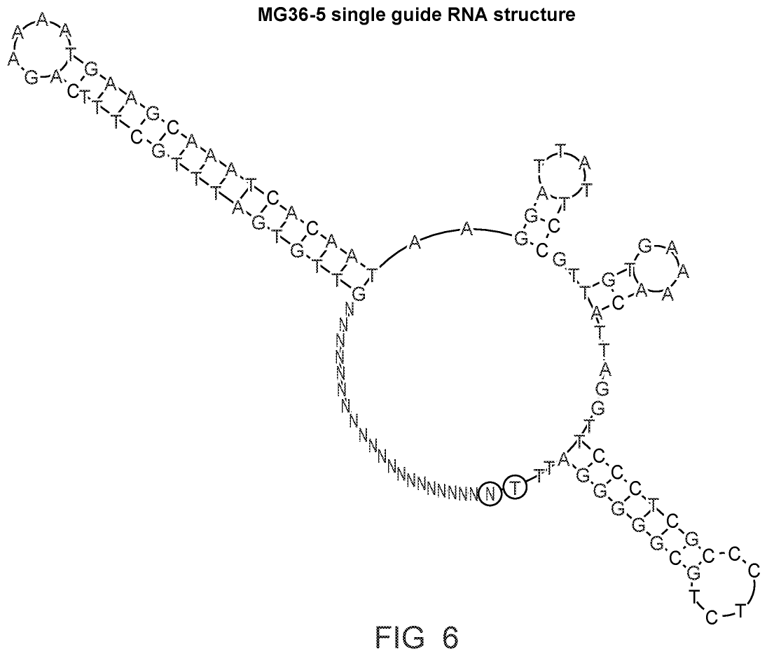

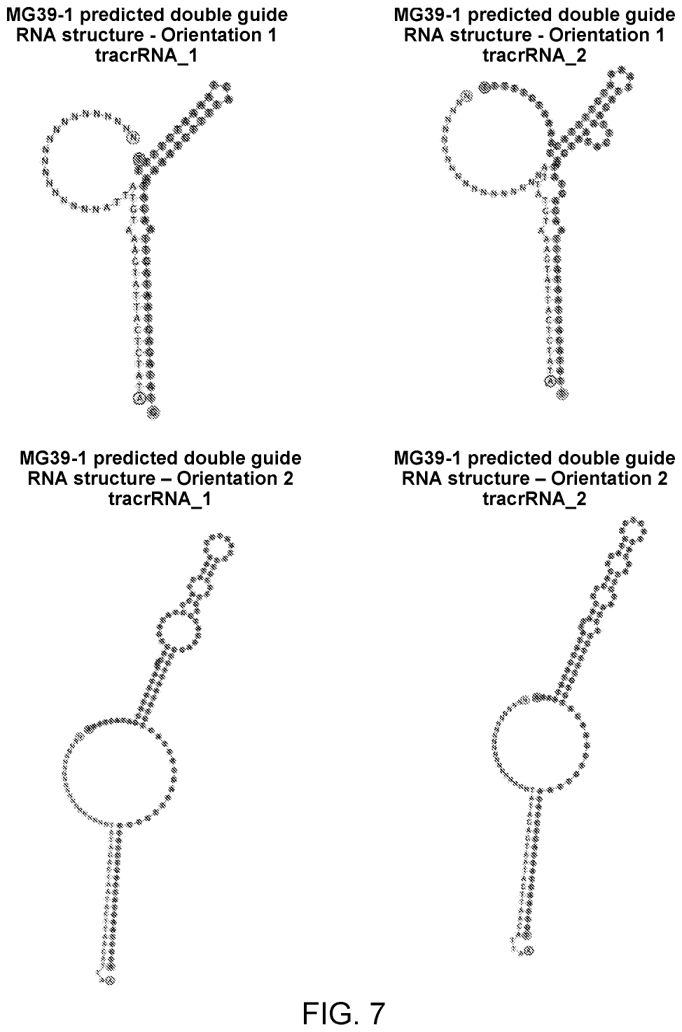

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein (e.g., MG36 or MG39 systems described herein). In some embodiments, such conservatively substituted variants are functional variants. Such functional variants can encompass sequences with substitutions such that the activity of critical active site residues of the endonuclease is not disrupted. In some embodiments, a functional variant of any of the systems described herein lack substitution of at least one of the conserved or functional residues called out in FIGS. 4 and 5. In some embodiments, a functional variant of any of the systems described herein lacks substitution of all of the conserved or functional residues called out in FIGS. 4 and 5.

Conservative substitution tables providing functionally similar amino acids are available from a variety of references (see, for example, Creighton, Proteins: Structures and Molecular Properties (W H Freeman & Co.; 2^{nd} Edition (December 1993))). The following eight groups each contain amino acids that are conservative substitutions for one another:
1) Alaninc (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Scrine(S), Threonine (T); and
8) Cysteine (C), Methionine (M).

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “recombinase” refers to an enzyme that mediates the recombination of DNA fragments located between recombinase recognition sequences, which results in the excision, insertion, inversion, exchange, or translocation of the DNA fragments located between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the excision, insertion, inversion, exchange, or translocation of a nucleic acid sequence, e.g., in or between one or more nucleic acid molecules.

As used herein, the term “transposon,” or “transposable element” refers to a nucleic acid sequence in a genome that is a mobile genetic element that can change its position in a genome. In some cases, the transposon transports additional “cargo DNA” excised from the genome. Transposons comprise, for example retrotransposons, DNA transposons, autonomous and non-autonomous transposons, and class III transposons. Transposon nucleic acid sequences comprise, for example genes coding for a cognate transposase, one or more recognition sequences for the transposase, or combinations thereof. In some cases, these transposons differ on the type of nucleic acid to transpose, the type of repeat at the ends of the transposon, the type of cargo to be carried or by the mode of transposition (i.e. self-repair or host-repair). As used herein, the term “transposase” or “transposases” refers to an enzyme that binds to the recognition sequences of a transposon and catalyzes its movement to another part of the genome. In some cases, the movement is by a cut and paste mechanism or a replicative transposition

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

As used herein, the term “complex” refers to a joining of at least two components. The two components may each retain the properties/activities they had prior to forming the complex. The joining may be by covalent bonding, non-covalent bonding (i.e., hydrogen bonding, ionic interactions, Van der Waals interactions, and hydrophobic bond), use of a linker, fusion, or any other suitable method. In some cases, components in a complex are polynucleotides, polypeptides, or combinations thereof. For example, a complex may comprise a Cas protein and a guide nucleic acid.

In some cases, the CAST systems described herein comprise one or more Tn7 or Tn7 like transposases. In certain example embodiments, the Tn7 or Tn7 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

In some cases, the CAST systems described herein comprise one or more Tn5053 or Tn5053 like transposases. In certain example embodiments, the Tn5053 or Tn5053 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

As used herein, the term “Cas12k” (alternatively “class 2, type V-K”) refers to a subtype of Type V CRISPR systems that have been found to be defective in nuclease activity (e.g., they may comprise at least one defective RuvC domain that lacking at least one catalytic residue important for DNA cleavage). Such subtype of effectors have been generally associated with CAST systems.

As used herein, the term “type I-F” (alternatively class 1, type I-F CRISPR) refers to a subtype of class 1, type I CRISPR systems. Such systems generally comprise multi-component CRISPR effectors comprising Cas8, Cas7, and Cas6 proteins. In some cases, such systems are found associated with CAST systems. In some cases, type I-F CRISPR systems comprise crRNAs comprising an 8-nt 5′ handles for Cas8 and/or Cas5 binding, 32-nt spacers bound by six copies of Cas7 for target recognition, or a 20-nt 3′ hairpins for Cas6 binding and pre-crRNA processing. In some cases, type-F systems utilize a 5′-CC PAM on the non-target strand for target binding.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

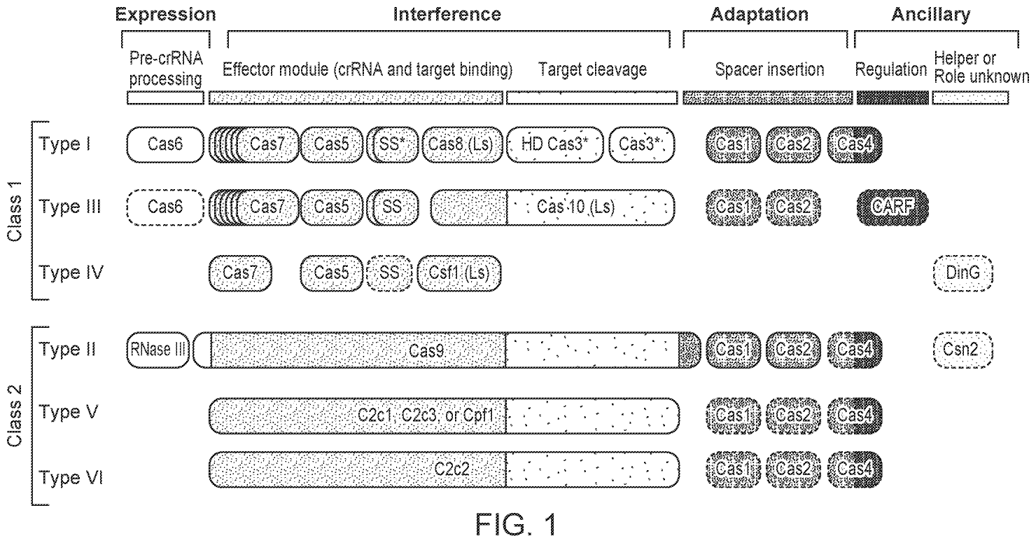

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class 1 CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that consists of a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class 2 CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRIPSR-Cas systems have RNA-guided RNA endonucleases. Instead of RuvC-like domains, the single polypeptide effector of Type VI systems (e.g., Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class 2 CRISPR-Cas have been most widely adopted for engineering and development as designer nuclease/genome editing applications.

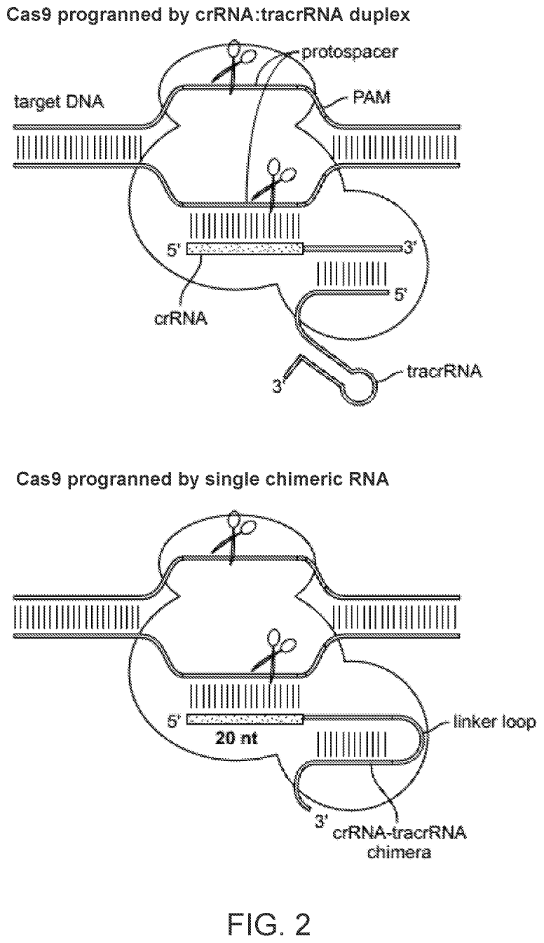

One of the early adaptations of such a system for in vitro use involved (i) recombinantly-expressed, purified full-length Cas9 (e.g., a Class 2, Type II Cas enzyme) isolated from S. pyogenes SF370, (ii) purified mature ˜42 nt crRNA bearing a ˜20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg2+. A later improved, engineered system involved the crRNA of (ii) joined to the 5′ end of (iii) by a linker (e.g., GAAA) to form a single fused synthetic guide RNA (sgRNA) capable of directing Cas9 to a target by itself (compare top and bottom panel of FIG. 2).

Such engineered systems can be adapted for use in mammalian cells by providing DNA vectors encoding (i) an ORF encoding codon-optimized Cas9 (e.g., a Class 2, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Transposons are mobile elements that can move between positions in a genome. Such transposons have evolved to limit the negative effects they exert on the host. A variety of regulatory mechanisms are used to maintain transposition at a low frequency and sometimes coordinate transposition with various cell processes. Some prokaryotic transposons also can mobilize functions that benefit the host or otherwise help maintain the element. Certain transposons may have also evolved mechanisms of tight control over target site selection, the most notable example being the Tn7 family.

Transposon Tn7 and similar elements may be reservoirs for antibiotic resistance and pathogenesis functions in clinical settings, as well as encoding other adaptive functions in natural environments. The Tn7 system, for example, has evolved mechanisms to almost completely avoid integrating into important host genes, but also maximize dispersal of the element by recognizing mobile plasmids and bacteriophage capable of moving Tn7 between host bacteria.

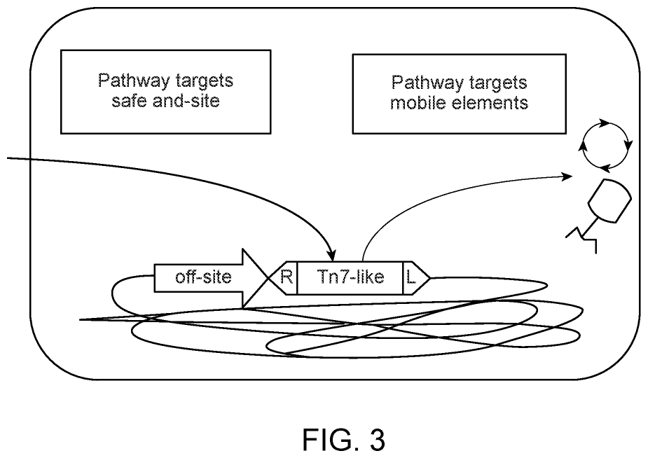

Tn7 and Tn7-like elements may control where and when they insert, possessing one pathway that directs insertion into a single conserved position in bacterial genomes and a second pathway that appears to be adapted to maximizing targeting into mobile plasmids capable of transporting the element between bacteria (see FIG. 3). The association between Tn7-like transposons and CRISPR-Cas systems suggests that the transposons might have hijacked CRISPR effectors to generate R-loops in target sites and facilitate the spread of transposons via plasmids and phages.

### MG36 Systems

Provided herein, in some embodiments, are MG36 systems for transposing a cargo nucleotide sequence into a target nucleic acid site. See FIGS. 4A-4C. In some embodiments, the system comprises a double-stranded nucleic acid. In some embodiments, this cargo nucleotide sequence is configured to interact with a recombinase complex. In some embodiments, the system comprises a Cas effector complex. In some embodiments, the Cas effector complex comprises a class 2, type II Cas effector and at least one engineered guide polynucleotide configured to hybridize to the target nucleic acid site. In some embodiments, the class 2, type II Cas effector comprises a RuvC domain and an HNH domain. In some embodiments, the system comprises the recombinase or transposase complex, wherein the recombinase or transposase complex is configured to recruit the cargo nucleotide sequence to the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class 2, type II Cas effector. In some cases, the class 2, type II Cas effector comprises a polypeptide which has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1. In some cases, the class 2, type II Cas effector comprises a polypeptide comprising a sequence having at least about 70% identity to SEQ ID NO: 1. In some cases, the class 2, type II Cas effector comprises a polypeptide comprising a sequence having at least about 75% identity to SEQ ID NO: 1. In some cases, the class 2, type II Cas effector comprises a polypeptide comprising a sequence having at least about 80% identity to SEQ ID NO: 1. In some cases, the class 2, type II Cas effector comprises a polypeptide comprising a sequence having at least about 85% identity to SEQ ID NO: 1. In some cases, the class 2, type II Cas effector comprises a polypeptide comprising a sequence having at least about 90% identity to SEQ ID NO: 1. In some cases, the class 2, type II Cas effector comprises a polypeptide comprising a sequence having at least about 91% identity to SEQ ID NO: 1. In some cases, the class 2, type II Cas effector comprises a polypeptide comprising a sequence having at least about 92% identity to SEQ ID NO: 1. In some cases, the class 2, type II Cas effector comprises a polypeptide comprising a sequence having at least about 93% identity to SEQ ID NO: 1. In some cases, the class 2, type II Cas effector comprises a polypeptide comprising a sequence having at least about 94% identity to SEQ ID NO: 1. In some cases, the class 2, type II Cas effector comprises a polypeptide comprising a sequence having at least about 95% identity to SEQ ID NO: 1. In some cases, the class 2, type II Cas effector comprises a polypeptide comprising a sequence having at least about 96% identity to SEQ ID NO: 1. In some cases, the class 2, type II Cas effector comprises a polypeptide comprising a sequence having at least about 97% identity to SEQ ID NO: 1. In some cases, the class 2, type II Cas effector comprises a polypeptide comprising a sequence having at least about 98% identity to SEQ ID NO: 1. In some cases, the class 2, type II Cas effector comprises a polypeptide comprising a sequence having at least about 99% identity to SEQ ID NO: 1. In some cases, the class 2, type II Cas effector comprises a polypeptide comprising a sequence having 100% identity to SEQ ID NO: 1.

In some cases, the recombinase or transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2-5. In some cases, the recombinase or transposase complex comprises at least one polypeptide comprising a sequence having at least about 70% identity to any one of SEQ ID NOs: 2-5. In some cases, the recombinase or transposase complex comprises at least one polypeptide comprising a sequence having at least about 75% identity to any one of SEQ ID NOs: 2-5. In some cases, the recombinase or transposase complex comprises at least one polypeptide comprising a sequence having at least about 80% identity to any one of SEQ ID NOs: 2-5. In some cases, the recombinase or transposase complex comprises at least one polypeptide comprising a sequence having at least about 85% identity to any one of SEQ ID NOs: 2-5. In some cases, the recombinase or transposase complex comprises at least one polypeptide comprising a sequence having at least about 90% identity to any one of SEQ ID NOs: 2-5. In some cases, the recombinase or transposase complex comprises at least one polypeptide comprising a sequence having at least about 91% identity to any one of SEQ ID NOs: 2-5. In some cases, the recombinase or transposase complex comprises at least one polypeptide comprising a sequence having at least about 92% identity to any one of SEQ ID NOs: 2-5. In some cases, the recombinase or transposase complex comprises at least one polypeptide comprising a sequence having at least about 93% identity to any one of SEQ ID NOs: 2-5. In some cases, the recombinase or transposase complex comprises at least one polypeptide comprising a sequence having at least about 94% identity to any one of SEQ ID NOs: 2-5. In some cases, the recombinase or transposase complex comprises at least one polypeptide comprising a sequence having at least about 95% identity to any one of SEQ ID NOs: 2-5. In some cases, the recombinase or transposase complex comprises at least one polypeptide comprising a sequence having at least about 96% identity to any one of SEQ ID NOs: 2-5. In some cases, the recombinase or transposase complex comprises at least one polypeptide comprising a sequence having at least about 97% identity to any one of SEQ ID NOs: 2-5. In some cases, the recombinase or transposase complex comprises at least one polypeptide comprising a sequence having at least about 98% identity to any one of SEQ ID NOs: 2-5. In some cases, the recombinase or transposase complex comprises at least one polypeptide comprising a sequence having at least about 99% identity to any one of SEQ ID NOs: 2-5. In some cases, the recombinase or transposase complex comprises at least one polypeptide comprising a sequence having 100% identity to any one of SEQ ID NOs: 2-5.

In some cases, the recombinase or transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2-5. In some cases, the recombinase or transposase complex comprises at least a first polypeptide and a second polypeptide each independently comprising a sequence having at least about 70% identity to any one of SEQ ID NOs: 2-5. In some cases, the recombinase or transposase complex comprises at least a first polypeptide and a second polypeptide each independently comprising a sequence having at least about 75% identity to any one of SEQ ID NOs: 2-5. In some cases, the recombinase or transposase complex comprises at least a first polypeptide and a second polypeptide each independently comprising a sequence having at least about 80% identity to any one of SEQ ID NOs: 2-5. In some cases, the recombinase or transposase complex comprises at least a first polypeptide and a second polypeptide each independently comprising a sequence having at least about 85% identity to any one of SEQ ID NOs: 2-5. In some cases, the recombinase or transposase complex comprises at least a first polypeptide and a second polypeptide each independently comprising a sequence having at least about 90% identity to any one of SEQ ID NOs: 2-5. In some cases, the recombinase or transposase complex comprises at least a first polypeptide and a second polypeptide each independently comprising a sequence having at least about 91% identity to any one of SEQ ID NOs: 2-5. In some cases, the recombinase or transposase complex comprises at least a first polypeptide and a second polypeptide each independently comprising a sequence having at least about 92% identity to any one of SEQ ID NOs: 2-5. In some cases, the recombinase or transposase complex comprises at least a first polypeptide and a second polypeptide each independently comprising a sequence having at least about 93% identity to any one of SEQ ID NOs: 2-5. In some cases, the recombinase or transposase complex comprises at least a first polypeptide and a second polypeptide each independently comprising a sequence having at least about 94% identity to any one of SEQ ID NOs: 2-5. In some cases, the recombinase or transposase complex comprises at least a first polypeptide and a second polypeptide each independently comprising a sequence having at least about 95% identity to any one of SEQ ID NOs: 2-5. In some cases, the recombinase or transposase complex comprises at least a first polypeptide and a second polypeptide each independently comprising a sequence having at least about 96% identity to any one of SEQ ID NOs: 2-5. In some cases, the recombinase or transposase complex comprises at least a first polypeptide and a second polypeptide each independently comprising a sequence having at least about 97% identity to any one of SEQ ID NOs: 2-5. In some cases, the recombinase or transposase complex comprises at least a first polypeptide and a second polypeptide each independently comprising a sequence having at least about 98% identity to any one of SEQ ID NOs: 2-5. In some cases, the recombinase or transposase complex comprises at least a first polypeptide and a second polypeptide each independently comprising a sequence having at least about 99% identity to any one of SEQ ID NOs: 2-5. In some cases, the recombinase or transposase complex comprises at least a first polypeptide and a second polypeptide each independently comprising a sequence having 100% identity to any one of SEQ ID NOs: 2-5.

In some cases, the recombinase or transposase complex comprises a TnsB1 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 2. In some cases, the recombinase or transposase complex comprises a TnsB1 polypeptide comprising a sequence having at least about 70% identity to SEQ ID NO: 2. In some cases, the recombinase or transposase complex comprises a TnsB1 polypeptide comprising a sequence having at least about 75% identity to SEQ ID NO: 2. In some cases, the recombinase or transposase complex comprises a TnsB1 polypeptide comprising a sequence having at least about 80% identity to SEQ ID NO: 2. In some cases, the recombinase or transposase complex comprises a TnsB1 polypeptide comprising a sequence having at least about 85% identity to SEQ ID NO: 2. In some cases, the recombinase or transposase complex comprises a TnsB1 polypeptide comprising a sequence having at least about 90% identity to SEQ ID NO: 2. In some cases, the recombinase or transposase complex comprises a TnsB1 polypeptide comprising a sequence having at least about 91% identity to SEQ ID NO: 2. In some cases, the recombinase or transposase complex comprises a TnsB1 polypeptide comprising a sequence having at least about 92% identity to SEQ ID NO: 2. In some cases, the recombinase or transposase complex comprises a TnsB1 polypeptide comprising a sequence having at least about 93% identity to SEQ ID NO: 2. In some cases, the recombinase or transposase complex comprises a TnsB1 polypeptide comprising a sequence having at least about 94% identity to SEQ ID NO: 2. In some cases, the recombinase or transposase complex comprises a TnsB1 polypeptide comprising a sequence having at least about 95% identity to SEQ ID NO: 2. In some cases, the recombinase or transposase complex comprises a TnsB1 polypeptide comprising a sequence having at least about 96% identity to SEQ ID NO: 2. In some cases, the recombinase or transposase complex comprises a TnsB1 polypeptide comprising a sequence having at least about 97% identity to SEQ ID NO: 2. In some cases, the recombinase or transposase complex comprises a TnsB1 polypeptide comprising a sequence having at least about 98% identity to SEQ ID NO: 2. In some cases, the recombinase or transposase complex comprises a TnsB1 polypeptide comprising a sequence having at least about 99% identity to SEQ ID NO: 2. In some cases, the recombinase or transposase complex comprises a TnsB1 polypeptide comprising a sequence having 100% identity to SEQ ID NO: 2.

In some cases, the recombinase or transposase complex comprises a TnsB2 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3. In some cases, the recombinase or transposase complex comprises a TnsB2 polypeptide comprising a sequence having at least about 70% identity to SEQ ID NO: 3. In some cases, the recombinase or transposase complex comprises a TnsB2 polypeptide comprising a sequence having at least about 75% identity to SEQ ID NO: 3. In some cases, the recombinase or transposase complex comprises a TnsB2 polypeptide comprising a sequence having at least about 80% identity to SEQ ID NO: 3. In some cases, the recombinase or transposase complex comprises a TnsB2 polypeptide comprising a sequence having at least about 85% identity to SEQ ID NO: 3. In some cases, the recombinase or transposase complex comprises a TnsB2 polypeptide comprising a sequence having at least about 90% identity to SEQ ID NO: 3. In some cases, the recombinase or transposase complex comprises a TnsB2 polypeptide comprising a sequence having at least about 91% identity to SEQ ID NO: 3. In some cases, the recombinase or transposase complex comprises a TnsB2 polypeptide comprising a sequence having at least about 92% identity to SEQ ID NO: 3. In some cases, the recombinase or transposase complex comprises a TnsB2 polypeptide comprising a sequence having at least about 93% identity to SEQ ID NO: 3. In some cases, the recombinase or transposase complex comprises a TnsB2 polypeptide comprising a sequence having at least about 94% identity to SEQ ID NO: 3. In some cases, the recombinase or transposase complex comprises a TnsB2 polypeptide comprising a sequence having at least about 95% identity to SEQ ID NO: 3. In some cases, the recombinase or transposase complex comprises a TnsB2 polypeptide comprising a sequence having at least about 96% identity to SEQ ID NO: 3. In some cases, the recombinase or transposase complex comprises a TnsB2 polypeptide comprising a sequence having at least about 97% identity to SEQ ID NO: 3. In some cases, the recombinase or transposase complex comprises a TnsB2 polypeptide comprising a sequence having at least about 98% identity to SEQ ID NO: 3. In some cases, the recombinase or transposase complex comprises a TnsB2 polypeptide comprising a sequence having at least about 99% identity to SEQ ID NO: 3. In some cases, the recombinase or transposase complex comprises a TnsB2 polypeptide comprising a sequence having 100% identity to SEQ ID NO: 3.

In some cases, the recombinase or transposase complex comprises a TnsT1 polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 4. In In some cases, the recombinase or transposase complex comprises a TnsT1 polypeptide comprising a sequence having at least about 70% identity to SEQ ID NO: 4. In some cases, the recombinase or transposase complex comprises a TnsT1 polypeptide comprising a sequence having at least about 75% identity to SEQ ID NO: 4. In some cases, the recombinase or transposase complex comprises a TnsT1 polypeptide comprising a sequence having at least about 80% identity to SEQ ID NO: 4. In some cases, the recombinase or transposase complex comprises a TnsT1 polypeptide comprising a sequence having at least about 85% identity to SEQ ID NO: 4. In some cases, the recombinase or transposase complex comprises a TnsT1 polypeptide comprising a sequence having at least about 90% identity to SEQ ID NO: 4. In some cases, the recombinase or transposase complex comprises a TnsT1 polypeptide comprising a sequence having at least about 91% identity to SEQ ID NO: 4. In some cases, the recombinase or transposase complex comprises a TnsT1 polypeptide comprising a sequence having at least about 92% identity to SEQ ID NO: 4. In some cases, the recombinase or transposase complex comprises a TnsT1 polypeptide comprising a sequence having at least about 93% identity to SEQ ID NO: 4. In some cases, the recombinase or transposase complex comprises a TnsT1 polypeptide comprising a sequence having at least about 94% identity to SEQ ID NO: 4. In some cases, the recombinase or transposase complex comprises a TnsT1 polypeptide comprising a sequence having at least about 95% identity to SEQ ID NO: 4. In some cases, the recombinase or transposase complex comprises a TnsT1 polypeptide comprising a sequence having at least about 96% identity to SEQ ID NO: 4. In some cases, the recombinase or transposase complex comprises a TnsT1 polypeptide comprising a sequence having at least about 97% identity to SEQ ID NO: 4. In some cases, the recombinase or transposase complex comprises a TnsT1 polypeptide comprising a sequence having at least about 98% identity to SEQ ID NO: 4. In some cases, the recombinase or transposase complex comprises a TnsT1 polypeptide comprising a sequence having at least about 99% identity to SEQ ID NO: 4. In some cases, the recombinase or transposase complex comprises a TnsT1 polypeptide comprising a sequence having 100% identity to SEQ ID NO: 4.

In some cases, the recombinase or transposase complex comprises a TnsC component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 5. In some cases, the recombinase or transposase complex comprises a TnsC component comprising a sequence having at least about 70% identity to SEQ ID NO: 5. In some cases, the recombinase or transposase complex comprises a TnsC component comprising a sequence having at least about 75% identity to SEQ ID NO: 5. In some cases, the recombinase or transposase complex comprises a TnsC component comprising a sequence having at least about 80% identity to SEQ ID NO: 5. In some cases, the recombinase or transposase complex comprises a TnsC component comprising a sequence having at least about 85% identity to SEQ ID NO: 5. In some cases, the recombinase or transposase complex comprises a TnsC component comprising a sequence having at least about 90% identity to SEQ ID NO: 5. In some cases, the recombinase or transposase complex comprises a TnsC component comprising a sequence having at least about 91% identity to SEQ ID NO: 5. In some cases, the recombinase or transposase complex comprises a TnsC component comprising a sequence having at least about 92% identity to SEQ ID NO: 5. In some cases, the recombinase or transposase complex comprises a TnsC component comprising a sequence having at least about 93% identity to SEQ ID NO: 5. In some cases, the recombinase or transposase complex comprises a TnsC component comprising a sequence having at least about 94% identity to SEQ ID NO: 5. In some cases, the recombinase or transposase complex comprises a TnsC component comprising a sequence having at least about 95% identity to SEQ ID NO: 5. In some cases, the recombinase or transposase complex comprises a TnsC component comprising a sequence having at least about 96% identity to SEQ ID NO: 5. In some cases, the recombinase or transposase complex comprises a TnsC component comprising a sequence having at least about 97% identity to SEQ ID NO: 5. In some cases, the recombinase or transposase complex comprises a TnsC component comprising a sequence having at least about 98% identity to SEQ ID NO: 5. In some cases, the recombinase or transposase complex comprises a TnsC component comprising a sequence having at least about 99% identity to SEQ ID NO: 5. In some cases, the recombinase or transposase complex comprises a TnsC component comprising a sequence having 100% identity to SEQ ID NO: 5.

In some embodiments, a system disclosed herein comprises at least one engineered guide polynucleotide, e.g., a gRNA.

In some embodiments, provided herein are engineered guide polynucleotides such as guide RNAs (gRNAs).

In some cases, the engineered guide polynucleotide comprises a sequence comprising at least 60-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 11. In some embodiments, the engineered guide polynucleotide comprises a sequence comprising at least 60-80 consecutive nucleotides at least about 70% to SEQ ID NO: 11. In some embodiments, the engineered guide polynucleotide comprises a sequence comprising at least 60-80 consecutive nucleotides at least about 75% to SEQ ID NO: 11. In some embodiments, the engineered guide polynucleotide comprises a sequence comprising at least 60-80 consecutive nucleotides at least about 80% to SEQ ID NO: 11. In some embodiments, the engineered guide polynucleotide comprises a sequence comprising at least 60-80 consecutive nucleotides at least about 85% to SEQ ID NO: 11. In some embodiments, the engineered guide polynucleotide comprises a sequence comprising at least 60-80 consecutive nucleotides at least about 90% to SEQ ID NO: 11. In some embodiments, the engineered guide polynucleotide comprises a sequence comprising at least 60-80 consecutive nucleotides at least about 91% to SEQ ID NO: 11. In some embodiments, the engineered guide polynucleotide comprises a sequence comprising at least 60-80 consecutive nucleotides at least about 92% to SEQ ID NO: 11. In some embodiments, the engineered guide polynucleotide comprises a sequence comprising at least 60-80 consecutive nucleotides at least about 93% to SEQ ID NO: 11. In some embodiments, the engineered guide polynucleotide comprises a sequence comprising at least 60-80 consecutive nucleotides at least about 94% to SEQ ID NO: 11. In some embodiments, the engineered guide polynucleotide comprises a sequence comprising at least 60-80 consecutive nucleotides at least about 95% to SEQ ID NO: 11. In some embodiments, the engineered guide polynucleotide comprises a sequence comprising at least 60-80 consecutive nucleotides at least about 96% to SEQ ID NO: 11. In some embodiments, the engineered guide polynucleotide comprises a sequence comprising at least 60-80 consecutive nucleotides at least about 97% to SEQ ID NO: 11. In some embodiments, the engineered guide polynucleotide comprises a sequence comprising at least 60-80 consecutive nucleotides at least about 98% to SEQ ID NO: 11. In some embodiments, the engineered guide polynucleotide comprises a sequence comprising at least 60-80 consecutive nucleotides at least about 99% to SEQ ID NO: 11. In some embodiments, the engineered guide polynucleotide comprises a sequence comprising at least 60-80 consecutive nucleotides 100% identical to SEQ ID NO: 11.

In some embodiments, the guide RNAs comprise various structural elements including but not limited to: a spacer sequence which binds to the protospacer sequence (target sequence), a crRNA, and an optional tracrRNA. In some embodiments, the guide RNA comprises a crRNA comprising a spacer sequence. In some embodiments, the guide RNA additionally comprises a tracrRNA or a modified tracrRNA.

In some embodiments, the systems provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a crRNA is part or considered part of a guide RNA, or is comprised in a guide RNA, e.g., a crRNA: tracrRNA chimera.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence. In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 17-18. In some cases, the left-hand recombinase sequence comprises a sequence having at least about 70% identity to any one of SEQ ID NOs: 17-18. In some cases, the left-hand recombinase sequence comprises a sequence having at least about 75% identity to any one of SEQ ID NOs: 17-18. In some cases, the left-hand recombinase sequence comprises a sequence having at least about 80% identity to any one of SEQ ID NOs: 17-18. In some cases, the left-hand recombinase sequence comprises a sequence having at least about 85% identity to any one of SEQ ID NOs: 17-18. In some cases, the left-hand recombinase sequence comprises a sequence having at least about 90% identity to any one of SEQ ID NOs: 17-18. In some cases, the left-hand recombinase sequence comprises a sequence having at least about 91% identity to any one of SEQ ID NOs: 17-18. In some cases, the left-hand recombinase sequence comprises a sequence having at least about 92% identity to any one of SEQ ID NOs: 17-18. In some cases, the left-hand recombinase sequence comprises a sequence having at least about 93% identity to any one of SEQ ID NOs: 17-18. In some cases, the left-hand recombinase sequence comprises a sequence having at least about 94% identity to any one of SEQ ID NOs: 17-18. In some cases, the left-hand recombinase sequence comprises a sequence having at least about 95% identity to any one of SEQ ID NOs: 17-18. In some cases, the left-hand recombinase sequence comprises a sequence having at least about 96% identity to any one of SEQ ID NOs: 17-18. In some cases, the left-hand recombinase sequence comprises a sequence having at least about 97% identity to any one of SEQ ID NOs: 17-18. In some cases, the left-hand recombinase sequence comprises a sequence having at least about 98% identity to any one of SEQ ID NOs: 17-18. In some cases, the left-hand recombinase sequence comprises a sequence having at least about 99% identity to any one of SEQ ID NOs: 17-18. In some cases, the left-hand recombinase sequence comprises a sequence having 100% identity to any one of SEQ ID NOs: 17-18.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 19. In some cases, the right-hand recombinase sequence comprises a sequence having at least about 70% identity to SEQ ID NO: 19. In some cases, the right-hand recombinase sequence comprises a sequence having at least about 75% identity to SEQ ID NO: 19. In some cases, the right-hand recombinase sequence comprises a sequence having at least about 80% identity to SEQ ID NO: 19. In some cases, the right-hand recombinase sequence comprises a sequence having at least about 85% identity to SEQ ID NO: 19. In some cases, the right-hand recombinase sequence comprises a sequence having at least about 90% identity to SEQ ID NO: 19. In some cases, the right-hand recombinase sequence comprises a sequence having at least about 91% identity to SEQ ID NO: 19. In some cases, the right-hand recombinase sequence comprises a sequence having at least about 92% identity to SEQ ID NO: 19. In some cases, the right-hand recombinase sequence comprises a sequence having at least about 93% identity to SEQ ID NO: 19. In some cases, the right-hand recombinase sequence comprises a sequence having at least about 94% identity to SEQ ID NO: 19. In some cases, the right-hand recombinase sequence comprises a sequence having at least about 95% identity to SEQ ID NO: 19. In some cases, the right-hand recombinase sequence comprises a sequence having at least about 96% identity to SEQ ID NO: 19. In some cases, the right-hand recombinase sequence comprises a sequence having at least about 97% identity to SEQ ID NO: 19. In some cases, the right-hand recombinase sequence comprises a sequence having at least about 98% identity to SEQ ID NO: 19. In some cases, the right-hand recombinase sequence comprises a sequence having at least about 99% identity to SEQ ID NO: 19. In some cases, the right-hand recombinase sequence comprises a sequence having 100% identity to SEQ ID NO: 19.

In some cases, the class 2, type II Cas effector and the recombinase or transposase complex are encoded by polynucleotide sequences comprising fewer than about 20 kilobases, fewer than about 15 kilobases, fewer than about 10 kilobases, or fewer than about 5 kilobases.

### MG39 Systems

Provided herein, in some embodiments, are MG39 systems for transposing a cargo nucleotide sequence into a target nucleic acid site. See FIGS. 5A-5B In some embodiments, the system comprises a double-stranded nucleic acid. In some embodiments, this cargo nucleotide sequence is configured to interact with a Tn7 type transposase complex. In some embodiments, the system comprises a Cas effector complex. In some embodiments, the Cas effector complex comprises a class 2, type V Cas effector and an engineered guide polynucleotide configured to hybridize to the target nucleotide sequence. In some embodiments, the class 2, type V Cas effector comprises a RuvC domain. In some embodiments, the system comprises the Tn7 type transposase complex configured to bind the Cas effector complex, wherein the Tn7 type transposase complex comprises a TnsA subunit.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class 2, type V Cas effector. In some cases, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 6. In some cases, the class 2, type V Cas effector comprises a polypeptide comprising a sequence having at least about 70% identity to SEQ ID NO: 6. In some cases, the class 2, type V Cas effector comprises a polypeptide comprising a sequence having at least about 75% identity to SEQ ID NO: 6. In some cases, the class 2, type V Cas effector comprises a polypeptide comprising a sequence having at least about 80% identity to SEQ ID NO: 6. In some cases, the class 2, type V Cas effector comprises a polypeptide comprising a sequence having at least about 85% identity to SEQ ID NO: 6. In some cases, the class 2, type V Cas effector comprises a polypeptide comprising a sequence having at least about 90% identity to SEQ ID NO: 6. In some cases, the class 2, type V Cas effector comprises a polypeptide comprising a sequence having at least about 91% identity to SEQ ID NO: 6. In some cases, the class 2, type V Cas effector comprises a polypeptide comprising a sequence having at least about 92% identity to SEQ ID NO: 6. In some cases, the class 2, type V Cas effector comprises a polypeptide comprising a sequence having at least about 93% identity to SEQ ID NO: 6. In some cases, the class 2, type V Cas effector comprises a polypeptide comprising a sequence having at least about 94% identity to SEQ ID NO: 6. In some cases, the class 2, type V Cas effector comprises a polypeptide comprising a sequence having at least about 95% identity to SEQ ID NO: 6. In some cases, the class 2, type V Cas effector comprises a polypeptide comprising a sequence having at least about 96% identity to SEQ ID NO: 6. In some cases, the class 2, type V Cas effector comprises a polypeptide comprising a sequence having at least about 97% identity to SEQ ID NO: 6. In some cases, the class 2, type V Cas effector comprises a polypeptide comprising a sequence having at least about 98% identity to SEQ ID NO: 6. In some cases, the class 2, type V Cas effector comprises a polypeptide comprising a sequence having at least about 99% identity to SEQ ID NO: 6. In some cases, the class 2, type V Cas effector comprises a polypeptide comprising a sequence having 100% identity to SEQ ID NO: 6.

In some cases, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8-10. In some cases, the Tn7 type transposase complex comprises at least one polypeptide comprising a sequence having at least about 70% identity to any one of SEQ ID NOs: 8-10. In some cases, the Tn7 type transposase complex comprises at least one polypeptide comprising a sequence having at least about 75% identity to any one of SEQ ID NOs: 8-10. In some cases, the Tn7 type transposase complex comprises at least one polypeptide comprising a sequence having at least about 80% identity to any one of SEQ ID NOs: 8-10. In some cases, the Tn7 type transposase complex comprises at least one polypeptide comprising a sequence having at least about 85% identity to any one of SEQ ID NOs: 8-10. In some cases, the Tn7 type transposase complex comprises at least one polypeptide comprising a sequence having at least about 90% identity to any one of SEQ ID NOs: 8-10. In some cases, the Tn7 type transposase complex comprises at least one polypeptide comprising a sequence having at least about 91% identity to any one of SEQ ID NOs: 8-10. In some cases, the Tn7 type transposase complex comprises at least one polypeptide comprising a sequence having at least about 92% identity to any one of SEQ ID NOs: 8-10. In some cases, the Tn7 type transposase complex comprises at least one polypeptide comprising a sequence having at least about 93% identity to any one of SEQ ID NOs: 8-10. In some cases, the Tn7 type transposase complex comprises at least one polypeptide comprising a sequence having at least about 94% identity to any one of SEQ ID NOs: 8-10. In some cases, the Tn7 type transposase complex comprises at least one polypeptide comprising a sequence having at least about 95% identity to any one of SEQ ID NOs: 8-10. In some cases, the Tn7 type transposase complex comprises at least one polypeptide comprising a sequence having at least about 96% identity to any one of SEQ ID NOs: 8-10. In some cases, the Tn7 type transposase complex comprises at least one polypeptide comprising a sequence having at least about 97% identity to any one of SEQ ID NOs: 8-10. In some cases, the Tn7 type transposase complex comprises at least one polypeptide comprising a sequence having at least about 98% identity to any one of SEQ ID NOs: 8-10. In some cases, the Tn7 type transposase complex comprises at least one polypeptide comprising a sequence having at least about 99% identity to any one of SEQ ID NOs: 8-10. In some cases, the Tn7 type transposase complex comprises at least one polypeptide comprising a sequence having 100% identity to any one of SEQ ID NOs: 8-10.

In some cases, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8-10. In some cases, the Tn7 type transposase complex comprises at least a first polypeptide and a second polypeptide each independently comprising a sequence having at least about 70% identity to any one of SEQ ID NOs: 8-10. In some cases, the Tn7 type transposase complex comprises at least a first polypeptide and a second polypeptide each independently comprising a sequence having at least about 75% identity to any one of SEQ ID NOs: 8-10. In some cases, the Tn7 type transposase complex comprises at least a first polypeptide and a second polypeptide each independently comprising a sequence having at least about 80% identity to any one of SEQ ID NOs: 8-10. In some cases, the Tn7 type transposase complex comprises at least a first polypeptide and a second polypeptide each independently comprising a sequence having at least about 85% identity to any one of SEQ ID NOs: 8-10. In some cases, the Tn7 type transposase complex comprises at least a first polypeptide and a second polypeptide each independently comprising a sequence having at least about 90% identity to any one of SEQ ID NOs: 8-10. In some cases, the Tn7 type transposase complex comprises at least a first polypeptide and a second polypeptide each independently comprising a sequence having at least about 91% identity to any one of SEQ ID NOs: 8-10. In some cases, the Tn7 type transposase complex comprises at least a first polypeptide and a second polypeptide each independently comprising a sequence having at least about 92% identity to any one of SEQ ID NOs: 8-10. In some cases, the Tn7 type transposase complex comprises at least a first polypeptide and a second polypeptide each independently comprising a sequence having at least about 93% identity to any one of SEQ ID NOs: 8-10. In some cases, the Tn7 type transposase complex comprises at least a first polypeptide and a second polypeptide each independently comprising a sequence having at least about 94% identity to any one of SEQ ID NOs: 8-10. In some cases, the Tn7 type transposase complex comprises at least a first polypeptide and a second polypeptide each independently comprising a sequence having at least about 95% identity to any one of SEQ ID NOs: 8-10. In some cases, the Tn7 type transposase complex comprises at least a first polypeptide and a second polypeptide each independently comprising a sequence having at least about 96% identity to any one of SEQ ID NOs: 8-10. In some cases, the Tn7 type transposase complex comprises at least a first polypeptide and a second polypeptide each independently comprising a sequence having at least about 97% identity to any one of SEQ ID NOs: 8-10. In some cases, the Tn7 type transposase complex comprises at least a first polypeptide and a second polypeptide each independently comprising a sequence having at least about 98% identity to any one of SEQ ID NOs: 8-10. In some cases, the Tn7 type transposase complex comprises at least a first polypeptide and a second polypeptide each independently comprising a sequence having at least about 99% identity to any one of SEQ ID NOs: 8-10. In some cases, the Tn7 type transposase complex comprises at least a first polypeptide and a second polypeptide each independently comprising a sequence having 100% identity to any one of SEQ ID NOs: 8-10.

In some cases, the Tn7 type transposase complex comprises a TnsA component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 7. In some cases, the Tn7 type transposase complex comprises a TnsA component comprising a sequence having at least about 70% identity to SEQ ID NO: 7. In some cases, the Tn7 type transposase complex comprises a TnsA component comprising a sequence having at least about 75% identity to SEQ ID NO: 7. In some cases, the Tn7 type transposase complex comprises a TnsA component comprising a sequence having at least about 80% identity to SEQ ID NO: 7. In some cases, the Tn7 type transposase complex comprises a TnsA component comprising a sequence having at least about 85% identity to SEQ ID NO: 7. In some cases, the Tn7 type transposase complex comprises a TnsA component comprising a sequence having at least about 90% identity to SEQ ID NO: 7. In some cases, the Tn7 type transposase complex comprises a TnsA component comprising a sequence having at least about 91% identity to SEQ ID NO: 7. In some cases, the Tn7 type transposase complex comprises a TnsA component comprising a sequence having at least about 92% identity to SEQ ID NO: 7. In some cases, the Tn7 type transposase complex comprises a TnsA component comprising a sequence having at least about 93% identity to SEQ ID NO: 7. In some cases, the Tn7 type transposase complex comprises a TnsA component comprising a sequence having at least about 94% identity to SEQ ID NO: 7. In some cases, the Tn7 type transposase complex comprises a TnsA component comprising a sequence having at least about 95% identity to SEQ ID NO: 7. In some cases, the Tn7 type transposase complex comprises a TnsA component comprising a sequence having at least about 96% identity to SEQ ID NO: 7. In some cases, the Tn7 type transposase complex comprises a TnsA component comprising a sequence having at least about 97% identity to SEQ ID NO: 7. In some cases, the Tn7 type transposase complex comprises a TnsA component comprising a sequence having at least about 98% identity to SEQ ID NO: 7. In some cases, the Tn7 type transposase complex comprises a TnsA component comprising a sequence having at least about 99% identity to SEQ ID NO: 7. In some cases, the Tn7 type transposase complex comprises a TnsA component comprising a sequence having 100% identity to SEQ ID NO: 7.

In some cases, the Tn7 type transposase complex comprises a TnsB component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 8. In some cases, the Tn7 type transposase complex comprises a TnsB component comprising a sequence having at least about 70% identity to SEQ ID NO: 8. In some cases, the Tn7 type transposase complex comprises a TnsB component comprising a sequence having at least about 75% identity to SEQ ID NO: 8. In some cases, the Tn7 type transposase complex comprises a TnsB component comprising a sequence having at least about 80% identity to SEQ ID NO: 8. In some cases, the Tn7 type transposase complex comprises a TnsB component comprising a sequence having at least about 85% identity to SEQ ID NO: 8. In some cases, the Tn7 type transposase complex comprises a TnsB component comprising a sequence having at least about 90% identity to SEQ ID NO: 8. In some cases, the Tn7 type transposase complex comprises a TnsB component comprising a sequence having at least about 91% identity to SEQ ID NO: 8. In some cases, the Tn7 type transposase complex comprises a TnsB component comprising a sequence having at least about 92% identity to SEQ ID NO: 8. In some cases, the Tn7 type transposase complex comprises a TnsB component comprising a sequence having at least about 93% identity to SEQ ID NO: 8. In some cases, the Tn7 type transposase complex comprises a TnsB component comprising a sequence having at least about 94% identity to SEQ ID NO: 8. In some cases, the Tn7 type transposase complex comprises a TnsB component comprising a sequence having at least about 95% identity to SEQ ID NO: 8. In some cases, the Tn7 type transposase complex comprises a TnsB component comprising a sequence having at least about 96% identity to SEQ ID NO: 8. In some cases, the Tn7 type transposase complex comprises a TnsB component comprising a sequence having at least about 97% identity to SEQ ID NO: 8. In some cases, the Tn7 type transposase complex comprises a TnsB component comprising a sequence having at least about 98% identity to SEQ ID NO: 8. In some cases, the Tn7 type transposase complex comprises a TnsB component comprising a sequence having at least about 99% identity to SEQ ID NO: 8. In some cases, the Tn7 type transposase complex comprises a TnsB component comprising a sequence having 100% identity to SEQ ID NO: 8.

In some cases, the Tn7 type transposase complex comprises a TnsC component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 9. In some cases, the Tn7 type transposase complex comprises a TnsC component comprising a sequence having at least about 70% identity to SEQ ID NO: 9. In some cases, the Tn7 type transposase complex comprises a TnsC component comprising a sequence having at least about 75% identity to SEQ ID NO: 9. In some cases, the Tn7 type transposasc complex comprises a TnsC component comprising a sequence having at least about 80% identity to SEQ ID NO: 9. In some cases, the Tn7 type transposase complex comprises a TnsC component comprising a sequence having at least about 85% identity to SEQ ID NO: 9. In some cases, the Tn7 type transposase complex comprises a TnsC component comprising a sequence having at least about 90% identity to SEQ ID NO: 9. In some cases, the Tn7 type transposase complex comprises a TnsC component comprising a sequence having at least about 91% identity to SEQ ID NO: 9. In some cases, the Tn7 type transposase complex comprises a TnsC component comprising a sequence having at least about 92% identity to SEQ ID NO: 9. In some cases, the Tn7 type transposase complex comprises a TnsC component comprising a sequence having at least about 93% identity to SEQ ID NO: 9. In some cases, the Tn7 type transposase complex comprises a TnsC component comprising a sequence having at least about 94% identity to SEQ ID NO: 9. In some cases, the Tn7 type transposase complex comprises a TnsC component comprising a sequence having at least about 95% identity to SEQ ID NO: 9. In some cases, the Tn7 type transposase complex comprises a TnsC component comprising a sequence having at least about 96% identity to SEQ ID NO: 9. In some cases, the Tn7 type transposase complex comprises a TnsC component comprising a sequence having at least about 97% identity to SEQ ID NO: 9. In some cases, the Tn7 type transposase complex comprises a TnsC component comprising a sequence having at least about 98% identity to SEQ ID NO: 9. In some cases, the Tn7 type transposase complex comprises a TnsC component comprising a sequence having at least about 99% identity to SEQ ID NO: 9. In some cases, the Tn7 type transposase complex comprises a TnsC component comprising a sequence having 100% identity to SEQ ID NO: 9.

In some cases, the Tn7 type transposase complex comprises a TniQ polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 10. In some cases, the Tn7 type transposase complex comprises a TniQ polypeptide comprising a sequence having at least about 70% identity to SEQ ID NO: 10. In some cases, the Tn7 type transposase complex comprises a TniQ polypeptide comprising a sequence having at least about 75% identity to SEQ ID NO: 10. In some cases, the Tn7 type transposase complex comprises a TniQ polypeptide comprising a sequence having at least about 80% identity to SEQ ID NO: 10. In some cases, the Tn7 type transposase complex comprises a TniQ polypeptide comprising a sequence having at least about 85% identity to SEQ ID NO: 10. In some cases, the Tn7 type transposase complex comprises a TniQ polypeptide comprising a sequence having at least about 90% identity to SEQ ID NO: 10. In some cases, the Tn7 type transposase complex comprises a TniQ polypeptide comprising a sequence having at least about 91% identity to SEQ ID NO: 10. In some cases, the Tn7 type transposase complex comprises a TniQ polypeptide comprising a sequence having at least about 92% identity to SEQ ID NO: 10. In some cases, the Tn7 type transposase complex comprises a TniQ polypeptide comprising a sequence having at least about 93% identity to SEQ ID NO: 10. In some cases, the Tn7 type transposase complex comprises a TniQ polypeptide comprising a sequence having at least about 94% identity to SEQ ID NO: 10. In some cases, the Tn7 type transposase complex comprises a TniQ polypeptide comprising a sequence having at least about 95% identity to SEQ ID NO: 10. In some cases, the Tn7 type transposase complex comprises a TniQ polypeptide comprising a sequence having at least about 96% identity to SEQ ID NO: 10. In some cases, the Tn7 type transposase complex comprises a TniQ polypeptide comprising a sequence having at least about 97% identity to SEQ ID NO: 10. In some cases, the Tn7 type transposase complex comprises a TniQ polypeptide comprising a sequence having at least about 98% identity to SEQ ID NO: 10. In some cases, the Tn7 type transposase complex comprises a TniQ polypeptide comprising a sequence having at least about 99% identity to SEQ ID NO: 10. In some cases, the Tn7 type transposase complex comprises a TniQ polypeptide comprising a sequence having 100% identity to SEQ ID NO: 10.

In some embodiments, a system disclosed herein comprises at least one engineered guide polynucleotide, e.g., a gRNA.

In some embodiments, provided herein are engineered guide polynucleotides such as guide RNAs (gRNAs).

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 13-16. In some embodiments, the engineered guide polynucleotide comprises a sequence comprising at least 46-80 consecutive nucleotides at least about 70% to any one of SEQ ID NOs: 13-16. In some embodiments, the engineered guide polynucleotide comprises a sequence comprising at least 46-80 consecutive nucleotides at least about 75% to any one of SEQ ID NOs: 13-16. In some embodiments, the engineered guide polynucleotide comprises a sequence comprising at least 46-80 consecutive nucleotides at least about 80% to any one of SEQ ID NOs: 13-16. In some embodiments, the engineered guide polynucleotide comprises a sequence comprising at least 46-80 consecutive nucleotides at least about 85% to any one of SEQ ID NOs: 13-16. In some embodiments, the engineered guide polynucleotide comprises a sequence comprising at least 46-80 consecutive nucleotides at least about 90% to any one of SEQ ID NOs: 13-16. In some embodiments, the engineered guide polynucleotide comprises a sequence comprising at least 46-80 consecutive nucleotides at least about 91% to any one of SEQ ID NOs: 13-16. In some embodiments, the engineered guide polynucleotide comprises a sequence comprising at least 46-80 consecutive nucleotides at least about 92% to any one of SEQ ID NOs: 13-16. In some embodiments, the engineered guide polynucleotide comprises a sequence comprising at least 46-80 consecutive nucleotides at least about 93% to any one of SEQ ID NOs: 13-16. In some embodiments, the engineered guide polynucleotide comprises a sequence comprising at least 46-80 consecutive nucleotides at least about 94% to any one of SEQ ID NOs: 13-16. In some embodiments, the engineered guide polynucleotide comprises a sequence comprising at least 46-80 consecutive nucleotides at least about 95% to any one of SEQ ID NOs: 13-16. In some embodiments, the engineered guide polynucleotide comprises a sequence comprising at least 46-80 consecutive nucleotides at least about 96% to any one of SEQ ID NOs: 13-16. In some embodiments, the engineered guide polynucleotide comprises a sequence comprising at least 46-80 consecutive nucleotides at least about 97% to any one of SEQ ID NOs: 13-16. In some embodiments, the engineered guide polynucleotide comprises a sequence comprising at least 46-80 consecutive nucleotides at least about 98% to any one of SEQ ID NOs: 13-16. In some embodiments, the engineered guide polynucleotide comprises a sequence comprising at least 46-80 consecutive nucleotides at least about 99% to any one of SEQ ID NOs: 13-16. In some embodiments, the engineered guide polynucleotide comprises a sequence comprising at least 46-80 consecutive nucleotides 100% identical to any one of SEQ ID NOs: 13-16.

In some embodiments, the guide RNAs comprise various structural elements including but not limited to: a spacer sequence which binds to the protospacer sequence (target sequence), a crRNA, and an optional tracrRNA. In some embodiments, the guide RNA comprises a crRNA comprising a spacer sequence. In some embodiments, the guide RNA additionally comprises a tracrRNA or a modified tracrRNA.

In some embodiments, the systems provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a crRNA is part or considered part of a guide RNA, or is comprised in a guide RNA, e.g., a crRNA: tracrRNA chimera.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 20. In some cases, the left-hand recombinase sequence comprises a sequence having at least about 70% identity to SEQ ID NO: 20. In some cases, the left-hand recombinase sequence comprises a sequence having at least about 75% identity to SEQ ID NO: 20. In some cases, the left-hand recombinase sequence comprises a sequence having at least about 80% identity to SEQ ID NO: 20. In some cases, the left-hand recombinase sequence comprises a sequence having at least about 85% identity to SEQ ID NO: 20. In some cases, the left-hand recombinase sequence comprises a sequence having at least about 90% identity to SEQ ID NO: 20. In some cases, the left-hand recombinase sequence comprises a sequence having at least about 91% identity to SEQ ID NO: 20. In some cases, the left-hand recombinase sequence comprises a sequence having at least about 92% identity to SEQ ID NO: 20. In some cases, the left-hand recombinase sequence comprises a sequence having at least about 93% identity to SEQ ID NO: 20. In some cases, the left-hand recombinase sequence comprises a sequence having at least about 94% identity to SEQ ID NO: 20. In some cases, the left-hand recombinase sequence comprises a sequence having at least about 95% identity to SEQ ID NO: 20. In some cases, the left-hand recombinase sequence comprises a sequence having at least about 96% identity to SEQ ID NO: 20. In some cases, the left-hand recombinase sequence comprises a sequence having at least about 97% identity to SEQ ID NO: 20. In some cases, the left-hand recombinase sequence comprises a sequence having at least about 98% identity to SEQ ID NO: 20. In some cases, the left-hand recombinase sequence comprises a sequence having at least about 99% identity to SEQ ID NO: 20. In some cases, the left-hand recombinase sequence comprises a sequence having 100% identity to SEQ ID NO: 20.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 21. In some cases, the right-hand recombinase sequence comprises a sequence having at least about 70% identity to SEQ ID NO: 21. In some cases, the right-hand recombinase sequence comprises a sequence having at least about 75% identity to SEQ ID NO: 21. In some cases, the right-hand recombinase sequence comprises a sequence having at least about 80% identity to SEQ ID NO: 21. In some cases, the right-hand recombinase sequence comprises a sequence having at least about 85% identity to SEQ ID NO: 21. In some cases, the right-hand recombinase sequence comprises a sequence having at least about 90% identity to SEQ ID NO: 21. In some cases, the right-hand recombinase sequence comprises a sequence having at least about 91% identity to SEQ ID NO: 21. In some cases, the right-hand recombinase sequence comprises a sequence having at least about 92% identity to SEQ ID NO: 21. In some cases, the right-hand recombinase sequence comprises a sequence having at least about 93% identity to SEQ ID NO: 21. In some cases, the right-hand recombinase sequence comprises a sequence having at least about 94% identity to SEQ ID NO: 21. In some cases, the right-hand recombinase sequence comprises a sequence having at least about 95% identity to SEQ ID NO: 21. In some cases, the right-hand recombinase sequence comprises a sequence having at least about 96% identity to SEQ ID NO: 21. In some cases, the right-hand recombinase sequence comprises a sequence having at least about 97% identity to SEQ ID NO: 21. In some cases, the right-hand recombinase sequence comprises a sequence having at least about 98% identity to SEQ ID NO: 21. In some cases, the right-hand recombinase sequence comprises a sequence having at least about 99% identity to SEQ ID NO: 21. In some cases, the right-hand recombinase sequence comprises a sequence having 100% identity to SEQ ID NO: 21.

In some cases, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### MG64 Systems

Provided herein, in some embodiments, are MG64 systems for transposing a cargo nucleotide sequence into a target nucleic acid site. In some embodiments, the system comprises a double-stranded nucleic acid comprising a cargo nucleotide sequence. In some embodiments, the cargo nucleotide sequence configured to interact with a Tn7 type or Tn5053 type transposase complex. In some embodiments, the system comprises a Cas effector complex. In some embodiments, the Cas effector complex comprises a class 2, type V Cas effector and an engineered guide polynucleotide configured to hybridize to the target nucleotide sequence. In some embodiments, the system comprises a Tn7 type or Tn5053 type transposase complex configured to bind the Cas effector complex. In some embodiments, the class 2, type V Cas effector comprises a RuvC domain.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site. In some cases, the PAM sequence comprises 5′-nGTn-3′ or 5′-nGTt-3′.

In some cases, the engineered guide polynucleotide is configured to bind the class 2, type V Cas effector. In some cases, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 22, 26, 30, 34, 55-89, 104, 147, 264-304, and 660-689. In some cases, the class 2, type V Cas effector comprises a polypeptide comprising a sequence having at least about 70% identity to any one of SEQ ID NOs: 22, 26, 30, 34, 55-89, 104, 147, 264-304, and 660-689. In some cases, the class 2, type V Cas effector comprises a polypeptide comprising a sequence having at least about 75% identity to any one of SEQ ID NOs: 22, 26, 30, 34, 55-89, 104, 147, 264-304, and 660-689. In some cases, the class 2, type V Cas effector comprises a polypeptide comprising a sequence having at least about 80% identity to any one of SEQ ID NOs: 22, 26, 30, 34, 55-89, 104, 147, 264-304, and 660-689. In some cases, the class 2, type V Cas effector comprises a polypeptide comprising a sequence having at least about 85% identity to any one of SEQ ID NOs: 22, 26, 30, 34, 55-89, 104, 147, 264-304, and 660-689. In some cases, the class 2, type V Cas effector comprises a polypeptide comprising a sequence having at least about 90% identity to any one of SEQ ID NOs: 22, 26, 30, 34, 55-89, 104, 147, 264-304, and 660-689. In some cases, the class 2, type V Cas effector comprises a polypeptide comprising a sequence having at least about 91% identity to any one of SEQ ID NOs: 22, 26, 30, 34, 55-89, 104, 147, 264-304, and 660-689. In some cases, the class 2, type V Cas effector comprises a polypeptide comprising a sequence having at least about 92% identity to any one of SEQ ID NOs: 22, 26, 30, 34, 55-89, 104, 147, 264-304, and 660-689. In some cases, the class 2, type V Cas effector comprises a polypeptide comprising a sequence having at least about 93% identity to any one of SEQ ID NOs: 22, 26, 30, 34, 55-89, 104, 147, 264-304, and 660-689. In some cases, the class 2, type V Cas effector comprises a polypeptide comprising a sequence having at least about 94% identity to any one of SEQ ID NOs: 22, 26, 30, 34, 55-89, 104, 147, 264-304, and 660-689. In some cases, the class 2, type V Cas effector comprises a polypeptide comprising a sequence having at least about 95% identity to any one of SEQ ID NOS: 22, 26, 30, 34, 55-89, 104, 147, 264-304, and 660-689. In some cases, the class 2, type V Cas effector comprises a polypeptide comprising a sequence having at least about 96% identity to any one of SEQ ID NOs: 22, 26, 30, 34, 55-89, 104, 147, 264-304, and 660-689. In some cases, the class 2, type V Cas effector comprises a polypeptide comprising a sequence having at least about 97% identity to any one of SEQ ID NOs: 22, 26, 30, 34, 55-89, 104, 147, 264-304, and 660-689. In some cases, the class 2, type V Cas effector comprises a polypeptide comprising a sequence having at least about 98% identity to any one of SEQ ID NOs: 22, 26, 30, 34, 55-89, 104, 147, 264-304, and 660-689. In some cases, the class 2, type V Cas effector comprises a polypeptide comprising a sequence having at least about 99% identity to any one of SEQ ID NOs: 22, 26, 30, 34, 55-89, 104, 147, 264-304, and 660-689. In some cases, the class 2, type V Cas effector comprises a polypeptide comprising a sequence having 100% identity to any one of SEQ ID NOs: 22, 26, 30, 34, 55-89, 104, 147, 264-304, and 660-689.

In some cases, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 70% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 75% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 80% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 85% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 90% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 91% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 92% identity to any one of SEQ ID NOS: 23-25, 27-29, 31-33, 35-37, 101-103, 105-107, 148-150, 305-343, and 345-347. In some cases, the Tn7 type transposase complex comprises at least one polypeptide comprising a sequence having at least about 93% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 94% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 95% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 96% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 97% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 98% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having at least about 99% identity to any one of SEQ ID NOs: 23-25, 27-29, 31-33, 35-37, 101-103, 105-107, 148-150, 305-343, and 345-347. In some cases, the Tn7 type transposase complex comprises at least one polypeptide comprising a sequence having 100% identity to any one of SEQ ID NOs: 23-25, 27-29, 31-33, 35-37, 101-103, 105-107, 148-150, 305-343, and 345-347. In some embodiments, the Tn7 type transposase complex comprises TnsB, TnsC, and TniQ

In some cases, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 70% identity to any one of SEQ ID NOS: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 75% identity to any one of SEQ ID NOS: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 80% identity to any one of SEQ ID NOS: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 85% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 90% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 91% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 92% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 93% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 94% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 95% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 96% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 97% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 98% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having at least about 99% identity to any one of SEQ ID NOs: 23-25, 27-29, 31-33, 35-37, 101-103, 105-107, 148-150, 305-343, and 345-347. In some cases, the Tn7 type transposase complex comprises at least a first polypeptide and a second polypeptide each independently comprising a sequence having 100% identity to any one of SEQ ID NOs: 23-25, 27-29, 31-33, 35-37, 101-103, 105-107, 148-150, 305-343, and 345-347.

In some embodiments, a system disclosed herein comprises at least one engineered guide polynucleotide, e.g., a gRNA.

In some embodiments, provided herein are engineered guide polynucleotides such as guide RNAs (gRNAs).

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 90-93, 117, 151, 156-181, 209-234, 417-460, and 715-739. In some cases, the engineered guide polynucleotide comprises a sequence comprising at least 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 70% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 75% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 80% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 85% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 90% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 91% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 92% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 93% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 94% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 95% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 96% to any one of SEQ ID NOs: 90-93, 117, 151, 156-181, 209-234, 417-460, and 715-739.

In some embodiments, the engineered guide polynucleotide comprises a sequence comprising at least 46-80 consecutive nucleotides at least about 97% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 98% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides at least about 99% to any one of SEQ ID NOs: 90-93, 117, 151, 156-181, 209-234, 417-460, and 715-739. In some embodiments, the engineered guide polynucleotide comprises a sequence comprising at least 46-80 consecutive nucleotides 100% identical to any one of SEQ ID NOs: 90-93, 117, 151, 156-181, 209-234, 417-460, and 715-739.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 111-114, 201-204, 262, 263, 348, 350-353, and 473-492.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 111-114, 201-204, 262, 263, 348, 350-353, and 473-492. In some embodiments, the engineered guide polynucleotide comprises a sequence having at least about 70% to any one of SEQ ID NOs: 111-114, 201-204, 262, 263, 348, 350-353, and 473-492. In some embodiments, the engineered guide polynucleotide comprises a sequence having at least about 75% to any one of SEQ ID NOs: 111-114, 201-204, 262, 263, 348, 350-353, and 473-492. In some embodiments, the engineered guide polynucleotide comprises a sequence having at least about 80% to any one of SEQ ID NOs: 111-114, 201-204, 262, 263, 348, 350-353, and 473-492. In some embodiments, the engineered guide polynucleotide comprises a sequence having at least about 85% to any one of SEQ ID NOs: 111-114, 201-204, 262, 263, 348, 350-353, and 473-492. In some embodiments, the engineered guide polynucleotide comprises a sequence having at least about 90% to any one of SEQ ID NOs: 111-114, 201-204, 262, 263, 348, 350-353, and 473-492. In some embodiments, the engineered guide polynucleotide comprises a sequence having at least about 91% to any one of SEQ ID NOs: 111-114, 201-204, 262, 263, 348, 350-353, and 473-492. In some embodiments, the engineered guide polynucleotide comprises a sequence having at least about 92% to any one of SEQ ID NOs: 111-114, 201-204, 262, 263, 348, 350-353, and 473-492. In some embodiments, the engineered guide polynucleotide comprises a sequence having at least about 93% to any one of SEQ ID NOs: 111-114, 201-204, 262, 263, 348, 350-353, and 473-492. In some embodiments, the engineered guide polynucleotide comprises a sequence having at least about 94% to any one of SEQ ID NOs: 111-114, 201-204, 262, 263, 348, 350-353, and 473-492. In some embodiments, the engineered guide polynucleotide comprises a sequence having at least about 95% to any one of SEQ ID NOs: 111-114, 201-204, 262, 263, 348, 350-353, and 473-492. In some embodiments, the engineered guide polynucleotide comprises a sequence having at least about 96% to any one of SEQ ID NOs: 111-114, 201-204, 262, 263, 348, 350-353, and 473-492. In some embodiments, the engineered guide polynucleotide comprises a sequence having at least about 97% to any one of SEQ ID NOs: 111-114, 201-204, 262, 263, 348, 350-353, and 473-492. In some embodiments, the engineered guide polynucleotide comprises a sequence having at least about 98% to any one of SEQ ID NOs: 111-114, 201-204, 262, 263, 348, 350-353, and 473-492. In some embodiments, the engineered guide polynucleotide comprises a sequence having at least about 99% to any one of SEQ ID NOs: 111-114, 201-204, 262, 263, 348, 350-353, and 473-492. In some embodiments, the engineered guide polynucleotide comprises a sequence having 100% identical to any one of SEQ ID NOs: 111-114, 201-204, 262, 263, 348, 350-353, and 473-492.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of any one of SEQ ID NOs: 123, 125, 127, 129, 131, 133, 153, 354-358, 461, 463, 465, and 467. In some cases, the left-hand recombinase sequence comprises a sequence having at least about 70% identity to any one of SEQ ID NOs: 123, 125, 127, 129, 131, 133, 153, 354-358, 461, 463, 465, and 467. In some cases, the left-hand recombinase sequence comprises a sequence having at least about 75% identity to any one of SEQ ID NOs: 123, 125, 127, 129, 131, 133, 153, 354-358, 461, 463, 465, and 467. In some cases, the left-hand recombinase sequence comprises a sequence having at least about 80% identity to any one of SEQ ID NOs: 123, 125, 127, 129, 131, 133, 153, 354-358, 461, 463, 465, and 467. In some cases, the left-hand recombinase sequence comprises a sequence having at least about 85% identity to any one of SEQ ID NOs: 123, 125, 127, 129, 131, 133, 153, 354-358, 461, 463, 465, and 467. In some cases, the left-hand recombinase sequence comprises a sequence having at least about 90% identity to any one of SEQ ID NOs: 123, 125, 127, 129, 131, 133, 153, 354-358, 461, 463, 465, and 467. In some cases, the left-hand recombinase sequence comprises a sequence having at least about 91% identity to any one of SEQ ID NOs: 123, 125, 127, 129, 131, 133, 153, 354-358, 461, 463, 465, and 467. In some cases, the left-hand recombinase sequence comprises a sequence having at least about 92% identity to any one of SEQ ID NOs: 123, 125, 127, 129, 131, 133, 153, 354-358, 461, 463, 465, and 467. In some cases, the left-hand recombinase sequence comprises a sequence having at least about 93% identity to any one of SEQ ID NOs: 123, 125, 127, 129, 131, 133, 153, 354-358, 461, 463, 465, and 467. In some cases, the left-hand recombinase sequence comprises a sequence having at least about 94% identity to any one of SEQ ID NOs: 123, 125, 127, 129, 131, 133, 153, 354-358, 461, 463, 465, and 467. In some cases, the left-hand recombinase sequence comprises a sequence having at least about 95% identity to any one of SEQ ID NOs: 123, 125, 127, 129, 131, 133, 153, 354-358, 461, 463, 465, and 467. In some cases, the left-hand recombinase sequence comprises a sequence having at least about 96% identity to any one of SEQ ID NOs: 123, 125, 127, 129, 131, 133, 153, 354-358, 461, 463, 465, and 467. In some cases, the left-hand recombinase sequence comprises a sequence having at least about 97% identity to any one of SEQ ID NOs: 123, 125, 127, 129, 131, 133, 153, 354-358, 461, 463, 465, and 467. In some cases, the left-hand recombinase sequence comprises a sequence having at least about 98% identity to any one of SEQ ID NOs: 123, 125, 127, 129, 131, 133, 153, 354-358, 461, 463, 465, and 467. In some cases, the left-hand recombinase sequence comprises a sequence having at least about 99% identity to any one of SEQ ID NOs: 123, 125, 127, 129, 131, 133, 153, 354-358, 461, 463, 465, and 467. In some cases, the left-hand recombinase sequence comprises a sequence having 100% identity to any one of SEQ ID NOs: 123, 125, 127, 129, 131, 133, 153, 354-358, 461, 463, 465, and 467.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 124, 126, 128, 130, 132, 154, 155, 359-363, 462, 464, 466, and 468. In some cases, the right-hand recombinase sequence comprises a sequence having at least about 70% identity to any one of SEQ ID NOs: 124, 126, 128, 130, 132, 154, 155, 359-363, 462, 464, 466, and 468. In some cases, the right-hand recombinase sequence comprises a sequence having at least about 75% identity to any one of SEQ ID NOs: 124, 126, 128, 130, 132, 154, 155, 359-363, 462, 464, 466, and 468. In some cases, the right-hand recombinase sequence comprises a sequence having at least about 80% identity to any one of SEQ ID NOs: 124, 126, 128, 130, 132, 154, 155, 359-363, 462, 464, 466, and 468. In some cases, the right-hand recombinase sequence comprises a sequence having at least about 85% identity to any one of SEQ ID NOs: 124, 126, 128, 130, 132, 154, 155, 359-363, 462, 464, 466, and 468. In some cases, the right-hand recombinase sequence comprises a sequence having at least about 90% identity to any one of SEQ ID NOs: 124, 126, 128, 130, 132, 154, 155, 359-363, 462, 464, 466, and 468. In some cases, the right-hand recombinase sequence comprises a sequence having at least about 91% identity to any one of SEQ ID NOs: 124, 126, 128, 130, 132, 154, 155, 359-363, 462, 464, 466, and 468. In some cases, the right-hand recombinase sequence comprises a sequence having at least about 92% identity to any one of SEQ ID NOs: 124, 126, 128, 130, 132, 154, 155, 359-363, 462, 464, 466, and 468. In some cases, the right-hand recombinase sequence comprises a sequence having at least about 93% identity to any one of SEQ ID NOs: 124, 126, 128, 130, 132, 154, 155, 359-363, 462, 464, 466, and 468. In some cases, the right-hand recombinase sequence comprises a sequence having at least about 94% identity to any one of SEQ ID NOs: 124, 126, 128, 130, 132, 154, 155, 359-363, 462, 464, 466, and 468. In some cases, the right-hand recombinase sequence comprises a sequence having at least about 95% identity to any one of SEQ ID NOs: 124, 126, 128, 130, 132, 154, 155, 359-363, 462, 464, 466, and 468. In some cases, the right-hand recombinase sequence comprises a sequence having at least about 96% identity to any one of SEQ ID NOs: 124, 126, 128, 130, 132, 154, 155, 359-363, 462, 464, 466, and 468. In some cases, the right-hand recombinase sequence comprises a sequence having at least about 97% identity to any one of SEQ ID NOs: 124, 126, 128, 130, 132, 154, 155, 359-363, 462, 464, 466, and 468. In some cases, the right-hand recombinase sequence comprises a sequence having at least about 98% identity to any one of SEQ ID NOs: 124, 126, 128, 130, 132, 154, 155, 359-363, 462, 464, 466, and 468. In some cases, the right-hand recombinase sequence comprises a sequence having at least about 99% identity to any one of SEQ ID NOs: 124, 126, 128, 130, 132, 154, 155, 359-363, 462, 464, 466, and 468. In some cases, the right-hand recombinase sequence comprises a sequence having 100% identity to any one of SEQ ID NOs: 124, 126, 128, 130, 132, 154, 155, 359-363, 462, 464, 466, and 468.

In some cases, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### MG108 Systems

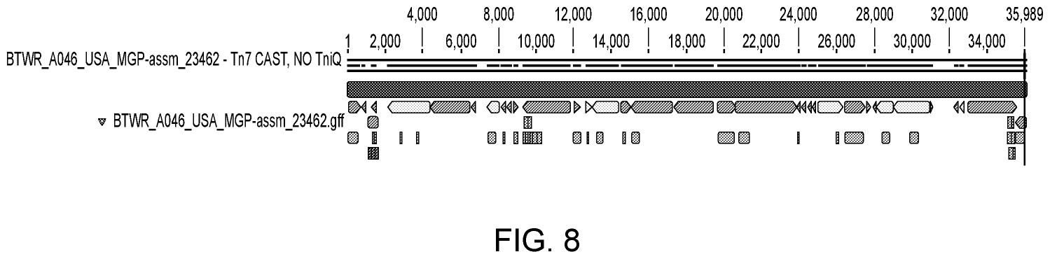

Provided herein, in some embodiments, are MG108 systems for transposing a cargo nucleotide sequence into a target nucleic acid site. See FIG. 8. In some embodiments, the system comprises a double-stranded nucleic acid comprising a cargo nucleotide sequence. In some embodiments, the cargo nucleotide sequence is configured to interact with a Tn7 type transposase complex. In some embodiments, the system comprises a Cas effector complex. In some embodiments, the Cas effector complex comprises a class 2, type V Cas effector and an engineered guide polynucleotide configured to hybridize to the target nucleotide sequence. In some embodiments, the class 2, type V Cas effector comprises a RuvC domain. In some embodiments, the system comprises a Tn7 type transposase complex configured to bind the Cas effector complex. In some cases, the Tn7 type transposase complex comprises TnsB and TnsC components but does not comprise a TnsA and/or TniQ component.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class 2, type V Cas effector. In some cases, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 38 or SEQ ID NO: 108. In some cases, the class 2, type V Cas effector comprises a polypeptide comprising a sequence having at least about 70% identity to SEQ ID NO: 38 or SEQ ID NO: 108. In some cases, the class 2, type V Cas effector comprises a polypeptide comprising a sequence having at least about 75% identity to SEQ ID NO: 38 or SEQ ID NO: 108. In some cases, the class 2, type V Cas effector comprises a polypeptide comprising a sequence having at least about 80% identity to SEQ ID NO: 38 or SEQ ID NO: 108. In some cases, the class 2, type V Cas effector comprises a polypeptide comprising a sequence having at least about 85% identity to SEQ ID NO: 38 or SEQ ID NO: 108. In some cases, the class 2, type V Cas effector comprises a polypeptide comprising a sequence having at least about 90% identity to SEQ ID NO: 38 or SEQ ID NO: 108. In some cases, the class 2, type V Cas effector comprises a polypeptide comprising a sequence having at least about 91% identity to SEQ ID NO: 38 or SEQ ID NO: 108. In some cases, the class 2, type V Cas effector comprises a polypeptide comprising a sequence having at least about 92% identity to SEQ ID NO: 38 or SEQ ID NO: 108. In some cases, the class 2, type V Cas effector comprises a polypeptide comprising a sequence having at least about 93% identity to SEQ ID NO: 38 or SEQ ID NO: 108. In some cases, the class 2, type V Cas effector comprises a polypeptide comprising a sequence having at least about 94% identity to SEQ ID NO: 38 or SEQ ID NO: 108. In some cases, the class 2, type V Cas effector comprises a polypeptide comprising a sequence having at least about 95% identity to SEQ ID NO: 38 or SEQ ID NO: 108. In some cases, the class 2, type V Cas effector comprises a polypeptide comprising a sequence having at least about 96% identity to SEQ ID NO: 38 or SEQ ID NO: 108. In some cases, the class 2, type V Cas effector comprises a polypeptide comprising a sequence having at least about 97% identity to SEQ ID NO: 38 or SEQ ID NO: 108. In some cases, the class 2, type V Cas effector comprises a polypeptide comprising a sequence having at least about 98% identity to SEQ ID NO: 38 or SEQ ID NO: 108. In some cases, the class 2, type V Cas effector comprises a polypeptide comprising a sequence having at least about 99% identity to SEQ ID NO: 38 or SEQ ID NO: 108. In some cases, the class 2, type V Cas effector comprises a polypeptide comprising a sequence having 100% identity to SEQ ID NO: 38 or SEQ ID NO: 108.

In some cases, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 39-40, 109-110, and 344. In some cases, the Tn7 type transposase complex comprises at least one polypeptide comprising a sequence having at least about 70% identity to any one of SEQ ID NO: 39-40, 109-110, and 344. In some cases, the Tn7 type transposase complex comprises at least one polypeptide comprising a sequence having at least about 75% identity to any one of SEQ ID NO: 39-40, 109-110, and 344. In some cases, the Tn7 type transposase complex comprises at least one polypeptide comprising a sequence having at least about 80% identity to any one of SEQ ID NO: 39-40, 109-110, and 344. In some cases, the Tn7 type transposase complex comprises at least one polypeptide comprising a sequence having at least about 85% identity to any one of SEQ ID NO: 39-40, 109-110, and 344. In some cases, the Tn7 type transposase complex comprises at least one polypeptide comprising a sequence having at least about 90% identity to any one of SEQ ID NO: 39-40, 109-110, and 344. In some cases, the Tn7 type transposase complex comprises at least one polypeptide comprising a sequence having at least about 91% identity to any one of SEQ ID NO: 39-40, 109-110, and 344. In some cases, the Tn7 type transposase complex comprises at least one polypeptide comprising a sequence having at least about 92% identity to any one of SEQ ID NO: 39-40, 109-110, and 344. In some cases, the Tn7 type transposase complex comprises at least one polypeptide comprising a sequence having at least about 93% identity to any one of SEQ ID NO: 39-40, 109-110, and 344. In some cases, the Tn7 type transposase complex comprises at least one polypeptide comprising a sequence having at least about 94% identity to any one of SEQ ID NO: 39-40, 109-110, and 344. In some cases, the Tn7 type transposase complex comprises at least one polypeptide comprising a sequence having at least about 95% identity to any one of SEQ ID NO: 39-40, 109-110, and 344. In some cases, the Tn7 type transposase complex comprises at least one polypeptide comprising a sequence having at least about 96% identity to any one of SEQ ID NO: 39-40, 109-110, and 344. In some cases, the Tn7 type transposase complex comprises at least one polypeptide comprising a sequence having at least about 97% identity to any one of SEQ ID NO: 39-40, 109-110, and 344. In some cases, the Tn7 type transposase complex comprises at least one polypeptide comprising a sequence having at least about 98% identity to any one of SEQ ID NO: 39-40, 109-110, and 344. In some cases, the Tn7 type transposase complex comprises at least one polypeptide comprising a sequence having at least about 99% identity to any one of SEQ ID NO: 39-40, 109-110, and 344. In some cases, the Tn7 type transposase complex comprises at least one polypeptide comprising a sequence having 100% identity to any one of SEQ ID NO: 39-40, 109-110, and 344.

In some cases, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 39-40, 109-110, and 344. In some cases, the Tn7 type transposase complex comprises at least a first polypeptide and a second polypeptide each independently comprising a sequence having at least about 70% identity to any one of SEQ ID NO: 39-40, 109-110, and 344. In some cases, the Tn7 type transposase complex comprises at least a first polypeptide and a second polypeptide each independently comprising a sequence having at least about 75% identity to any one of SEQ ID NO: 39-40, 109-110, and 344. In some cases, the Tn7 type transposase complex comprises at least a first polypeptide and a second polypeptide each independently comprising a sequence having at least about 80% identity to any one of SEQ ID NO: 39-40, 109-110, and 344. In some cases, the Tn7 type transposase complex comprises at least a first polypeptide and a second polypeptide each independently comprising a sequence having at least about 85% identity to any one of SEQ ID NO: 39-40, 109-110, and 344. In some cases, the Tn7 type transposase complex comprises at least a first polypeptide and a second polypeptide each independently comprising a sequence having at least about 90% identity to any one of SEQ ID NO: 39-40, 109-110, and 344. In some cases, the Tn7 type transposase complex comprises at least a first polypeptide and a second polypeptide each independently comprising a sequence having at least about 91% identity to any one of SEQ ID NO: 39-40, 109-110, and 344. In some cases, the Tn7 type transposase complex comprises at least a first polypeptide and a second polypeptide each independently comprising a sequence having at least about 92% identity to any one of SEQ ID NO: 39-40, 109-110, and 344. In some cases, the Tn7 type transposase complex comprises at least a first polypeptide and a second polypeptide each independently comprising a sequence having at least about 93% identity to any one of SEQ ID NO: 39-40, 109-110, and 344. In some cases, the Tn7 type transposase complex comprises at least a first polypeptide and a second polypeptide each independently comprising a sequence having at least about 94% identity to any one of SEQ ID NO: 39-40, 109-110, and 344. In some cases, the Tn7 type transposase complex comprises at least a first polypeptide and a second polypeptide each independently comprising a sequence having at least about 95% identity to any one of SEQ ID NO: 39-40, 109-110, and 344. In some cases, the Tn7 type transposase complex comprises at least a first polypeptide and a second polypeptide each independently comprising a sequence having at least about 96% identity to any one of SEQ ID NO: 39-40, 109-110, and 344. In some cases, the Tn7 type transposase complex comprises at least a first polypeptide and a second polypeptide each independently comprising a sequence having at least about 97% identity to any one of SEQ ID NO: 39-40, 109-110, and 344. In some cases, the Tn7 type transposase complex comprises at least a first polypeptide and a second polypeptide each independently comprising a sequence having at least about 98% identity to any one of SEQ ID NO: 39-40, 109-110, and 344. In some cases, the Tn7 type transposase complex comprises at least a first polypeptide and a second polypeptide each independently comprising a sequence having at least about 99% identity to any one of SEQ ID NO: 39-40, 109-110, and 344. In some cases, the Tn7 type transposase complex comprises at least a first polypeptide and a second polypeptide each independently comprising a sequence having 100% identity to any one of SEQ ID NO: 39-40, 109-110, and 344.

In some cases, the Tn7 type transposase complex comprises a TnsB component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 40 and 109. In some cases, the Tn7 type transposase complex comprises a TnsB component comprising a sequence having at least about 70% identity to any one of SEQ ID NOs: 40 and 109. In some cases, the Tn7 type transposase complex comprises a TnsB component comprising a sequence having at least about 75% identity to any one of SEQ ID NOs: 40 and 109. In some cases, the Tn7 type transposase complex comprises a TnsB component comprising a sequence having at least about 80% identity to any one of SEQ ID NOs: 40 and 109. In some cases, the Tn7 type transposase complex comprises a TnsB component comprising a sequence having at least about 85% identity to any one of SEQ ID NOs: 40 and 109. In some cases, the Tn7 type transposase complex comprises a TnsB component comprising a sequence having at least about 90% identity to any one of SEQ ID NOs: 40 and 109. In some cases, the Tn7 type transposase complex comprises a TnsB component comprising a sequence having at least about 91% identity to any one of SEQ ID NOs: 40 and 109. In some cases, the Tn7 type transposase complex comprises a TnsB component comprising a sequence having at least about 92% identity to any one of SEQ ID NOs: 40 and 109. In some cases, the Tn7 type transposase complex comprises a TnsB component comprising a sequence having at least about 93% identity to any one of SEQ ID NOs: 40 and 109. In some cases, the Tn7 type transposase complex comprises a TnsB component comprising a sequence having at least about 94% identity to any one of SEQ ID NOs: 40 and 109. In some cases, the Tn7 type transposase complex comprises a TnsB component comprising a sequence having at least about 95% identity to any one of SEQ ID NOs: 40 and 109. In some cases, the Tn7 type transposase complex comprises a TnsB component comprising a sequence having at least about 96% identity to any one of SEQ ID NOs: 40 and 109. In some cases, the Tn7 type transposase complex comprises a TnsB component comprising a sequence having at least about 97% identity to any one of SEQ ID NOs: 40 and 109. In some cases, the Tn7 type transposase complex comprises a TnsB component comprising a sequence having at least about 98% identity to any one of SEQ ID NOs: 40 and 109. In some cases, the Tn7 type transposase complex comprises a TnsB component comprising a sequence having at least about 99% identity to any one of SEQ ID NOs: 40 and 109. In some cases, the Tn7 type transposase complex comprises a TnsB component comprising a sequence having 100% identity to any one of SEQ ID NOs: 40 and 109.

In some cases, the Tn7 type transposase complex comprises a TnsC component comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of any one of SEQ ID NOs: 39 and 110. In some cases, the Tn7 type transposasc complex comprises a TnsC component comprising a sequence having at least about 70% identity to any one of SEQ ID NOs: 39 and 110. In some cases, the Tn7 type transposase complex comprises a TnsC component comprising a sequence having at least about 75% identity to any one of SEQ ID NOs: 39 and 110. In some cases, the Tn7 type transposase complex comprises a TnsC component comprising a sequence having at least about 80% identity to any one of SEQ ID NOs: 39 and 110. In some cases, the Tn7 type transposase complex comprises a TnsC component comprising a sequence having at least about 85% identity to any one of SEQ ID NOs: 39 and 110. In some cases, the Tn7 type transposase complex comprises a TnsC component comprising a sequence having at least about 90% identity to any one of SEQ ID NOs: 39 and 110. In some cases, the Tn7 type transposase complex comprises a TnsC component comprising a sequence having at least about 91% identity to any one of SEQ ID NOs: 39 and 110. In some cases, the Tn7 type transposase complex comprises a TnsC component comprising a sequence having at least about 92% identity to any one of SEQ ID NOs: 39 and 110. In some cases, the Tn7 type transposase complex comprises a TnsC component comprising a sequence having at least about 93% identity to any one of SEQ ID NOs: 39 and 110. In some cases, the Tn7 type transposase complex comprises a TnsC component comprising a sequence having at least about 94% identity to any one of SEQ ID NOs: 39 and 110. In some cases, the Tn7 type transposase complex comprises a TnsC component comprising a sequence having at least about 95% identity to any one of SEQ ID NOs: 39 and 110. In some cases, the Tn7 type transposase complex comprises a TnsC component comprising a sequence having at least about 96% identity to any one of SEQ ID NOs: 39 and 110. In some cases, the Tn7 type transposase complex comprises a TnsC component comprising a sequence having at least about 97% identity to any one of SEQ ID NOs: 39 and 110. In some cases, the Tn7 type transposase complex comprises a TnsC component comprising a sequence having at least about 98% identity to any one of SEQ ID NOs: 39 and 110. In some cases, the Tn7 type transposase complex comprises a TnsC component comprising a sequence having at least about 99% identity to any one of SEQ ID NOs: 39 and 110. In some cases, the Tn7 type transposase complex comprises a TnsC component comprising a sequence having 100% identity to any one of SEQ ID NOs: 39 and 110.

In some cases, the Tn7 type transposase complex comprises TnsB and TnsC components comprising sequences having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 40 and 39 or 109 and 110, or a variant thereof, respectively. In some cases, the Tn7 type transposase complex comprises TnsB and TnsC components comprising sequences substantially identical to any one of SEQ ID NOs: 40 and 39 or 109 and 110, or a variant thereof, respectively.

In some embodiments, a system disclosed herein comprises at least one engineered guide polynucleotide, e.g., a gRNA.

In some embodiments, provided herein are engineered guide polynucleotides such as guide RNAs (gRNAs).

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 118, 182, 183, 235, and 236. In some embodiments, the engineered guide polynucleotide comprises a sequence comprising at least 46-80 consecutive nucleotides at least about 70% to any one of SEQ ID NOs: 118, 182, 183, 235, and 236. In some embodiments, the engineered guide polynucleotide comprises a sequence comprising at least 46-80 consecutive nucleotides at least about 75% to any one of SEQ ID NOS: 118, 182, 183, 235, and 236. In some embodiments, the engineered guide polynucleotide comprises a sequence comprising at least 46-80 consecutive nucleotides at least about 80% to any one of SEQ ID NOs: 118, 182, 183, 235, and 236. In some embodiments, the engineered guide polynucleotide comprises a sequence comprising at least 46-80 consecutive nucleotides at least about 85% to any one of SEQ ID NOs: 118, 182, 183, 235, and 236. In some embodiments, the engineered guide polynucleotide comprises a sequence comprising at least 46-80 consecutive nucleotides at least about 90% to any one of SEQ ID NOs: 118, 182, 183, 235, and 236. In some embodiments, the engineered guide polynucleotide comprises a sequence comprising at least 46-80 consecutive nucleotides at least about 91% to any one of SEQ ID NOs: 118, 182, 183, 235, and 236. In some embodiments, the engineered guide polynucleotide comprises a sequence comprising at least 46-80 consecutive nucleotides at least about 92% to any one of SEQ ID NOs: 118, 182, 183, 235, and 236. In some embodiments, the engineered guide polynucleotide comprises a sequence comprising at least 46-80 consecutive nucleotides at least about 93% to any one of SEQ ID NOs: 118, 182, 183, 235, and 236. In some embodiments, the engineered guide polynucleotide comprises a sequence comprising at least 46-80 consecutive nucleotides at least about 94% to any one of SEQ ID NOS: 118, 182, 183, 235, and 236. In some embodiments, the engineered guide polynucleotide comprises a sequence comprising at least 46-80 consecutive nucleotides at least about 95% to any one of SEQ ID NOs: 118, 182, 183, 235, and 236. In some embodiments, the engineered guide polynucleotide comprises a sequence comprising at least 46-80 consecutive nucleotides at least about 96% to any one of SEQ ID NOs: 118, 182, 183, 235, and 236. In some embodiments, the engineered guide polynucleotide comprises a sequence comprising at least 46-80 consecutive nucleotides at least about 97% to any one of SEQ ID NOs: 118, 182, 183, 235, and 236. In some embodiments, the engineered guide polynucleotide comprises a sequence comprising at least 46-80 consecutive nucleotides at least about 98% to any one of SEQ ID NOs: 118, 182, 183, 235, and 236. In some embodiments, the engineered guide polynucleotide comprises a sequence comprising at least 46-80 consecutive nucleotides at least about 99% to any one of SEQ ID NOs: 118, 182, 183, 235, and 236. In some embodiments, the engineered guide polynucleotide comprises a sequence comprising at least 46-80 consecutive nucleotides 100% identical to any one of SEQ ID NOs: 118, 182, 183, 235, and 236.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 115-116, 205-206, and 493, or a variant thereof. In some cases, the engineered guide polynucleotide comprises a sequence comprising at least about 46-80 consecutive nucleotides substantially identical to the non-degenerate nucleotides of any one of SEQ ID NOs: 115-116, 205-206, and 493, or a variant thereof.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 115-116, 205-206, and 493. In some embodiments, the engineered guide polynucleotide comprises a sequence having at least about 70% to any one of SEQ ID NOs: 115-116, 205-206, and 493. In some embodiments, the engineered guide polynucleotide comprises a sequence having at least about 75% to any one of SEQ ID NOs: 115-116, 205-206, and 493. In some embodiments, the engineered guide polynucleotide comprises a sequence having at least about 80% to any one of SEQ ID NOs: 115-116, 205-206, and 493. In some embodiments, the engineered guide polynucleotide comprises a sequence having at least about 85% to any one of SEQ ID NOs: 115-116, 205-206, and 493. In some embodiments, the engineered guide polynucleotide comprises a sequence having at least about 90% to any one of SEQ ID NOs: 115-116, 205-206, and 493. In some embodiments, the engineered guide polynucleotide comprises a sequence having at least about 91% to any one of SEQ ID NOs: 115-116, 205-206, and 493. In some embodiments, the engineered guide polynucleotide comprises a sequence having at least about 92% to any one of SEQ ID NOs: 115-116, 205-206, and 493. In some embodiments, the engineered guide polynucleotide comprises a sequence having at least about 93% to any one of SEQ ID NOs: 115-116, 205-206, and 493. In some embodiments, the engineered guide polynucleotide comprises a sequence having at least about 94% to any one of SEQ ID NOs: 115-116, 205-206, and 493. In some embodiments, the engineered guide polynucleotide comprises a sequence having at least about 95% to any one of SEQ ID NOs: 115-116, 205-206, and 493. In some embodiments, the engineered guide polynucleotide comprises a sequence having at least about 96% to any one of SEQ ID NOs: 115-116, 205-206, and 493. In some embodiments, the engineered guide polynucleotide comprises a sequence having at least about 97% to any one of SEQ ID NOs: 115-116, 205-206, and 493. In some embodiments, the engineered guide polynucleotide comprises a sequence having at least about 98% to any one of SEQ ID NOs: 115-116, 205-206, and 493. In some embodiments, the engineered guide polynucleotide comprises a sequence having at least about 99% to any one of SEQ ID NOs: 115-116, 205-206, and 493. In some embodiments, the engineered guide polynucleotide comprises a sequence having 100% identical to any one of SEQ ID NOs: 115-116, 205-206, and 493.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or

nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 134. In some cases, the left-hand recombinase sequence comprises a sequence having at least about 70% identity to SEQ ID NO: 134. In some cases, the left-hand recombinase sequence comprises a sequence having at least about 75% identity to SEQ ID NO: 134. In some cases, the left-hand recombinase sequence comprises a sequence having at least about 80% identity to SEQ ID NO: 134. In some cases, the left-hand recombinase sequence comprises a sequence having at least about 85% identity to SEQ ID NO: 134. In some cases, the left-hand recombinase sequence comprises a sequence having at least about 90% identity to SEQ ID NO: 134. In some cases, the left-hand recombinase sequence comprises a sequence having at least about 91% identity to SEQ ID NO: 134. In some cases, the left-hand recombinase sequence comprises a sequence having at least about 92% identity to SEQ ID NO: 134. In some cases, the left-hand recombinase sequence comprises a sequence having at least about 93% identity to SEQ ID NO: 134. In some cases, the left-hand recombinase sequence comprises a sequence having at least about 94% identity to SEQ ID NO: 134. In some cases, the left-hand recombinase sequence comprises a sequence having at least about 95% identity to SEQ ID NO: 134. In some cases, the left-hand recombinase sequence comprises a sequence having at least about 96% identity to SEQ ID NO: 134. In some cases, the left-hand recombinase sequence comprises a sequence having at least about 97% identity to SEQ ID NO: 134. In some cases, the left-hand recombinase sequence comprises a sequence having at least about 98% identity to SEQ ID NO: 134. In some cases, the left-hand recombinase sequence comprises a sequence having at least about 99% identity to SEQ ID NO: 134. In some cases, the left-hand recombinase sequence comprises a sequence having 100% identity to SEQ ID NO: 134.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 135. In some cases, the right-hand recombinase sequence comprises a sequence having at least about 70% identity to SEQ ID NO: 135. In some cases, the right-hand recombinase sequence comprises a sequence having at least about 75% identity to SEQ ID NO: 135. In some cases, the right-hand recombinase sequence comprises a sequence having at least about 80% identity to SEQ ID NO: 135. In some cases, the right-hand recombinase sequence comprises a sequence having at least about 85% identity to SEQ ID NO: 135. In some cases, the right-hand recombinase sequence comprises a sequence having at least about 90% identity to SEQ ID NO: 135. In some cases, the right-hand recombinase sequence comprises a sequence having at least about 91% identity to SEQ ID NO: 135. In some cases, the right-hand recombinase sequence comprises a sequence having at least about 92% identity to SEQ ID NO: 135. In some cases, the right-hand recombinase sequence comprises a sequence having at least about 93% identity to SEQ ID NO: 135. In some cases, the right-hand recombinase sequence comprises a sequence having at least about 94% identity to SEQ ID NO: 135. In some cases, the right-hand recombinase sequence comprises a sequence having at least about 95% identity to SEQ ID NO: 135. In some cases, the right-hand recombinase sequence comprises a sequence having at least about 96% identity to SEQ ID NO: 135. In some cases, the right-hand recombinase sequence comprises a sequence having at least about 97% identity to SEQ ID NO: 135. In some cases, the right-hand recombinase sequence comprises a sequence having at least about 98% identity to SEQ ID NO: 135. In some cases, the right-hand recombinase sequence comprises a sequence having at least about 99% identity to SEQ ID NO: 135. In some cases, the right-hand recombinase sequence comprises a sequence having 100% identity to SEQ ID NO: 135.

In some cases, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### MG110 Systems

Provided herein, in some embodiments, are MG110 systems for transposing a cargo nucleotide sequence into a target nucleic acid site. In some embodiments, the system comprises a double-stranded nucleic acid comprising a cargo nucleotide sequence. In some embodiments, the cargo nucleotide sequence is configured to interact with a Tn7 type transposase complex. In some embodiments, the system comprises a Cas effector complex. In some embodiments, the Cas effector complex comprises a class I, type I Cas effector and an engineered guide polynucleotide configured to hybridize to the target nucleotide sequence. In some embodiments, the system comprises a Tn7 type transposase complex configured to bind the Cas effector complex.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence.

In some cases, a target nucleic acid comprises the target nucleic acid site. In some cases, the target nucleic acid comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

In some cases, the engineered guide polynucleotide is configured to bind the class 1, type I Cas effector. In some cases, the class 1, type I Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 41-43 and 48-50. In some cases, the class 1, type I Cas effector comprises a polypeptide comprising a sequence having at least about 70% identity to any one of SEQ ID NOs: 41-43 and 48-50. In some cases, the class 1, type I Cas effector comprises a polypeptide comprising a sequence having at least about 75% identity to any one of SEQ ID NOs: 41-43 and 48-50. In some cases, the class 1, type I Cas effector comprises a polypeptide comprising a sequence having at least about 80% identity to any one of SEQ ID NOs: 41-43 and 48-50. In some cases, the class 1, type I Cas effector comprises a polypeptide comprising a sequence having at least about 85% identity to any one of SEQ ID NOs: 41-43 and 48-50. In some cases, the class 1, type I Cas effector comprises a polypeptide comprising a sequence having at least about 90% identity to any one of SEQ ID NOs: 41-43 and 48-50. In some cases, the class 1, type I Cas effector comprises a polypeptide comprising a sequence having at least about 91% identity to any one of SEQ ID NOS: 41-43 and 48-50. In some cases, the class 1, type I Cas effector comprises a polypeptide comprising a sequence having at least about 92% identity to any one of SEQ ID NOs: 41-43 and 48-50. In some cases, the class 1, type I Cas effector comprises a polypeptide comprising a sequence having at least about 93% identity to any one of SEQ ID NOs: 41-43 and 48-50. In some cases, the class 1, type I Cas effector comprises a polypeptide comprising a sequence having at least about 94% identity to any one of SEQ ID NOs: 41-43 and 48-50. In some cases, the class 1, type I Cas effector comprises a polypeptide comprising a sequence having at least about 95% identity to any one of SEQ ID NOs: 41-43 and 48-50. In some cases, the class 1, type I Cas effector comprises a polypeptide comprising a sequence having at least about 96% identity to any one of SEQ ID NOs: 41-43 and 48-50. In some cases, the class 1, type I Cas effector comprises a polypeptide comprising a sequence having at least about 97% identity to any one of SEQ ID NOs: 41-43 and 48-50. In some cases, the class 1, type I Cas effector comprises a polypeptide comprising a sequence having at least about 98% identity to any one of SEQ ID NOs: 41-43 and 48-50. In some cases, the class 1, type I Cas effector comprises a polypeptide comprising a sequence having at least about 99% identity to any one of SEQ ID NOS: 41-43 and 48-50. In some cases, the class 1, type I Cas effector comprises a polypeptide comprising a sequence having 100% identity to any one of SEQ ID NOs: 41-43 and 48-50.

In some cases, the engineered guide polynucleotide is configured to bind the class 1, type I Cas effector. In some cases, the class 1, type I Cas effector comprises Cas6, Cas7, and Cas8 effectors comprising sequences having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 41-43 and 48-50. In some cases, the class 1, type I Cas effector comprises Cas6, Cas7, and Cas8 effectors comprising sequences substantially identical to any one of SEQ ID NOs: 41-43 and 48-50.

In some cases, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47 and 51-54. In some cases, the Tn7 type transposase complex comprises at least one polypeptide comprising a sequence having at least about 70% identity to any one of SEQ ID NOS: 44-47 and 51-54. In some cases, the Tn7 type transposase complex comprises at least one polypeptide comprising a sequence having at least about 75% identity to any one of SEQ ID NOS: 44-47 and 51-54. In some cases, the Tn7 type transposase complex comprises at least one polypeptide comprising a sequence having at least about 80% identity to any one of SEQ ID NOS: 44-47 and 51-54. In some cases, the Tn7 type transposase complex comprises at least one polypeptide comprising a sequence having at least about 85% identity to any one of SEQ ID NOs: 44-47 and 51-54. In some cases, the Tn7 type transposase complex comprises at least one polypeptide comprising a sequence having at least about 90% identity to any one of SEQ ID NOs: 44-47 and 51-54. In some cases, the Tn7 type transposase complex comprises at least one polypeptide comprising a sequence having at least about 91% identity to any one of SEQ ID NOS: 44-47 and 51-54. In some cases, the Tn7 type transposase complex comprises at least one polypeptide comprising a sequence having at least about 92% identity to any one of SEQ ID NOS: 44-47 and 51-54. In some cases, the Tn7 type transposase complex comprises at least one polypeptide comprising a sequence having at least about 93% identity to any one of SEQ ID NOS: 44-47 and 51-54. In some cases, the Tn7 type transposase complex comprises at least one polypeptide comprising a sequence having at least about 94% identity to any one of SEQ ID NOS: 44-47 and 51-54. In some cases, the Tn7 type transposase complex comprises at least one polypeptide comprising a sequence having at least about 95% identity to any one of SEQ ID NOS: 44-47 and 51-54. In some cases, the Tn7 type transposase complex comprises at least one polypeptide comprising a sequence having at least about 96% identity to any one of SEQ ID NOS: 44-47 and 51-54. In some cases, the Tn7 type transposase complex comprises at least one polypeptide comprising a sequence having at least about 97% identity to any one of SEQ ID NOs: 44-47 and 51-54. In some cases, the Tn7 type transposase complex comprises at least one polypeptide comprising a sequence having at least about 98% identity to any one of SEQ ID NOS: 44-47 and 51-54. In some cases, the Tn7 type transposase complex comprises at least one polypeptide comprising a sequence having at least about 99% identity to any one of SEQ ID NOS: 44-47 and 51-54. In some cases, the Tn7 type transposase complex comprises at least one polypeptide comprising a sequence having 100% identity to any one of SEQ ID NOs: 44-47 and 51-54.

In some cases, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 44-47 and 51-54. In some cases, the Tn7 type transposase complex comprises at least a first polypeptide and a second polypeptide each independently comprising a sequence having at least about 70% identity to any one of SEQ ID NOs: 44-47 and 51-54. In some cases, the Tn7 type transposase complex comprises at least a first polypeptide and a second polypeptide each independently comprising a sequence having at least about 75% identity to any one of SEQ ID NOs: 44-47 and 51-54. In some cases, the Tn7 type transposase complex comprises at least a first polypeptide and a second polypeptide each independently comprising a sequence having at least about 80% identity to any one of SEQ ID NOs: 44-47 and 51-54. In some cases, the Tn7 type transposase complex comprises at least a first polypeptide and a second polypeptide each independently comprising a sequence having at least about 85% identity to any one of SEQ ID NOs: 44-47 and 51-54. In some cases, the Tn7 type transposase complex comprises at least a first polypeptide and a second polypeptide each independently comprising a sequence having at least about 90% identity to any one of SEQ ID NOS: 44-47 and 51-54. In some cases, the Tn7 type transposase complex comprises at least a first polypeptide and a second polypeptide each independently comprising a sequence having at least about 91% identity to any one of SEQ ID NOs: 44-47 and 51-54. In some cases, the Tn7 type transposase complex comprises at least a first polypeptide and a second polypeptide each independently comprising a sequence having at least about 92% identity to any one of SEQ ID NOs: 44-47 and 51-54. In some cases, the Tn7 type transposase complex comprises at least a first polypeptide and a second polypeptide each independently comprising a sequence having at least about 93% identity to any one of SEQ ID NOs: 44-47 and 51-54. In some cases, the Tn7 type transposase complex comprises at least a first polypeptide and a second polypeptide each independently comprising a sequence having at least about 94% identity to any one of SEQ ID NOs: 44-47 and 51-54. In some cases, the Tn7 type transposase complex comprises at least a first polypeptide and a second polypeptide each independently comprising a sequence having at least about 95% identity to any one of SEQ ID NOs: 44-47 and 51-54. In some cases, the Tn7 type transposase complex comprises at least a first polypeptide and a second polypeptide each independently comprising a sequence having at least about 96% identity to any one of SEQ ID NOs: 44-47 and 51-54. In some cases, the Tn7 type transposase complex comprises at least a first polypeptide and a second polypeptide each independently comprising a sequence having at least about 97% identity to any one of SEQ ID NOs: 44-47 and 51-54. In some cases, the Tn7 type transposase complex comprises at least a first polypeptide and a second polypeptide each independently comprising a sequence having at least about 98% identity to any one of SEQ ID NOs: 44-47 and 51-54. In some cases, the Tn7 type transposase complex comprises at least a first polypeptide and a second polypeptide each independently comprising a sequence having at least about 99% identity to any one of SEQ ID NOs: 44-47 and 51-54. In some cases, the Tn7 type transposase complex comprises at least a first polypeptide and a second polypeptide each independently comprising a sequence having 100% identity to any one of SEQ ID NOs: 44-47 and 51-54. In some cases, the Tn7 type transposase complex comprises TnsA, TnsB, TnsC, and TniQ components.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to non-degenerate nucleotides of any one of SEQ ID NOs: 121, 122, 207, and 208, or a variant thereof. In some cases, the engineered guide polynucleotide comprises a sequence comprising at least about 46-80 consecutive nucleotides substantially identical to the non-degenerate nucleotides of any one of SEQ ID NOs: 121, 122, 207, and 208.

In some embodiments, a system disclosed herein comprises at least one engineered guide polynucleotide, e.g., a gRNA.

In some embodiments, provided herein are engineered guide polynucleotides such as guide RNAs (gRNAs).

In some cases, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 121, 122, 207, and 208. In some embodiments, the engineered guide polynucleotide comprises a sequence comprising at least 46-80 consecutive nucleotides at least about 70% to any one of SEQ ID NOs: 121, 122, 207, and 208. In some embodiments, the engineered guide polynucleotide comprises a sequence comprising at least 46-80 consecutive nucleotides at least about 75% to any one of SEQ ID NOs: 121, 122, 207, and 208. In some embodiments, the engineered guide polynucleotide comprises a sequence comprising at least 46-80 consecutive nucleotides at least about 80% to any one of SEQ ID NOS: 121, 122, 207, and 208. In some embodiments, the engineered guide polynucleotide comprises a sequence comprising at least 46-80 consecutive nucleotides at least about 85% to any one of SEQ ID NOs: 121, 122, 207, and 208. In some embodiments, the engineered guide polynucleotide comprises a sequence comprising at least 46-80 consecutive nucleotides at least about 90% to any one of SEQ ID NOs: 121, 122, 207, and 208. In some embodiments, the engineered guide polynucleotide comprises a sequence comprising at least 46-80 consecutive nucleotides at least about 91% to any one of SEQ ID NOs: 121, 122, 207, and 208. In some embodiments, the engineered guide polynucleotide comprises a sequence comprising at least 46-80 consecutive nucleotides at least about 92% to any one of SEQ ID NOs: 121, 122, 207, and 208. In some embodiments, the engineered guide polynucleotide comprises a sequence comprising at least 46-80 consecutive nucleotides at least about 93% to any one of SEQ ID NOs: 121, 122, 207, and 208. In some embodiments, the engineered guide polynucleotide comprises a sequence comprising at least 46-80 consecutive nucleotides at least about 94% to any one of SEQ ID NOs: 121, 122, 207, and 208. In some embodiments, the engineered guide polynucleotide comprises a sequence comprising at least 46-80 consecutive nucleotides at least about 95% to any one of SEQ ID NOs: 121, 122, 207, and 208. In some embodiments, the engineered guide polynucleotide comprises a sequence comprising at least 46-80 consecutive nucleotides at least about 96% to any one of SEQ ID NOs: 121, 122, 207, and 208. In some embodiments, the engineered guide polynucleotide comprises a sequence comprising at least 46-80 consecutive nucleotides at least about 97% to any one of SEQ ID NOs: 121, 122, 207, and 208. In some embodiments, the engineered guide polynucleotide comprises a sequence comprising at least 46-80 consecutive nucleotides at least about 98% to any one of SEQ ID NOs: 121, 122, 207, and 208. In some embodiments, the engineered guide polynucleotide comprises a sequence comprising at least 46-80 consecutive nucleotides at least about 99% to any one of SEQ ID NOs: 121, 122, 207, and 208. In some embodiments, the engineered guide polynucleotide comprises a sequence comprising at least 46-80 consecutive nucleotides 100% identical to any one of SEQ ID NOs: 121, 122, 207, and 208.

In some cases, the engineered guide polynucleotide comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 121, 122, 207, and 208. In some embodiments, the engineered guide polynucleotide comprises a sequence having at least about 70% to any one of SEQ ID NOs: 121, 122, 207, and 208. In some embodiments, the engineered guide polynucleotide comprises a sequence having at least about 75% to any one of SEQ ID NOs: 121, 122, 207, and 208. In some embodiments, the engineered guide polynucleotide comprises a sequence having at least about 80% to any one of SEQ ID NOs: 121, 122, 207, and 208. In some embodiments, the engineered guide polynucleotide comprises a sequence having at least about 85% to any one of SEQ ID NOs: 121, 122, 207, and 208. In some embodiments, the engineered guide polynucleotide comprises a sequence having at least about 90% to any one of SEQ ID NOs: 121, 122, 207, and 208. In some embodiments, the engineered guide polynucleotide comprises a sequence having at least about 91% to any one of SEQ ID NOs: 121, 122, 207, and 208. In some embodiments, the engineered guide polynucleotide comprises a sequence having at least about 92% to any one of SEQ ID NOs: 121, 122, 207, and 208. In some embodiments, the engineered guide polynucleotide comprises a sequence having at least about 93% to any one of SEQ ID NOs: 121, 122, 207, and 208. In some embodiments, the engineered guide polynucleotide comprises a sequence having at least about 94% to any one of SEQ ID NOs: 121, 122, 207, and 208. In some embodiments, the engineered guide polynucleotide comprises a sequence having at least about 95% to any one of SEQ ID NOs: 121, 122, 207, and 208. In some embodiments, the engineered guide polynucleotide comprises a sequence having at least about 96% to any one of SEQ ID NOs: 121, 122, 207, and 208. In some embodiments, the engineered guide polynucleotide comprises a sequence having at least about 97% to any one of SEQ ID NOs: 121, 122, 207, and 208. In some embodiments, the engineered guide polynucleotide comprises a sequence having at least about 98% to any one of SEQ ID NOs: 121, 122, 207, and 208. In some embodiments, the engineered guide polynucleotide comprises a sequence having at least about 99% to any one of SEQ ID NOs: 121, 122, 207, and 208. In some embodiments, the engineered guide polynucleotide comprises a sequence having 100% identical to any one of SEQ ID NOs: 121, 122, 207, and 208.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur(S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some cases, the left-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 136 or 138. In some cases, the left-hand recombinase sequence comprises a sequence having at least about 70% identity to SEQ ID NO: 136 or 138. In some cases, the left-hand recombinase sequence comprises a sequence having at least about 75% identity to SEQ ID NO: 136 or 138. In some cases, the left-hand recombinase sequence comprises a sequence having at least about 80% identity to SEQ ID NO: 136 or 138. In some cases, the left-hand recombinase sequence comprises a sequence having at least about 85% identity to SEQ ID NO: 136 or 138. In some cases, the left-hand recombinase sequence comprises a sequence having at least about 90% identity to SEQ ID NO: 136 or 138. In some cases, the left-hand recombinase sequence comprises a sequence having at least about 91% identity to SEQ ID NO: 136 or 138. In some cases, the left-hand recombinase sequence comprises a sequence having at least about 92% identity to SEQ ID NO: 136 or 138. In some cases, the left-hand recombinase sequence comprises a sequence having at least about 93% identity to SEQ ID NO: 136 or 138. In some cases, the left-hand recombinase sequence comprises a sequence having at least about 94% identity to SEQ ID NO: 136 or 138. In some cases, the left-hand recombinase sequence comprises a sequence having at least about 95% identity to SEQ ID NO: 136 or 138. In some cases, the left-hand recombinase sequence comprises a sequence having at least about 96% identity to SEQ ID NO: 136 or 138. In some cases, the left-hand recombinase sequence comprises a sequence having at least about 97% identity to SEQ ID NO: 136 or 138. In some cases, the left-hand recombinase sequence comprises a sequence having at least about 98% identity to SEQ ID NO: 136 or 138. In some cases, the left-hand recombinase sequence comprises a sequence having at least about 99% identity to SEQ ID NO: 136 or 138. In some cases, the left-hand recombinase sequence comprises a sequence having 100% identity to SEQ ID NO: 136 or 138.

In some cases, the right-hand recombinase sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 137 or 139. In some cases, the right-hand recombinase sequence comprises a sequence having at least about 70% identity to SEQ ID NO: 137 or 139. In some cases, the right-hand recombinase sequence comprises a sequence having at least about 75% identity to SEQ ID NO: 137 or 139. In some cases, the right-hand recombinase sequence comprises a sequence having at least about 80% identity to SEQ ID NO: 137 or 139. In some cases, the right-hand recombinase sequence comprises a sequence having at least about 85% identity to SEQ ID NO: 137 or 139. In some cases, the right-hand recombinase sequence comprises a sequence having at least about 90% identity to SEQ ID NO: 137 or 139. In some cases, the right-hand recombinase sequence comprises a sequence having at least about 91% identity to SEQ ID NO: 137 or 139. In some cases, the right-hand recombinase sequence comprises a sequence having at least about 92% identity to SEQ ID NO: 137 or 139. In some cases, the right-hand recombinase sequence comprises a sequence having at least about 93% identity to SEQ ID NO: 137 or 139. In some cases, the right-hand recombinase sequence comprises a sequence having at least about 94% identity to SEQ ID NO: 137 or 139. In some cases, the right-hand recombinase sequence comprises a sequence having at least about 95% identity to SEQ ID NO: 137 or 139. In some cases, the right-hand recombinase sequence comprises a sequence having at least about 96% identity to SEQ ID NO: 137 or 139. In some cases, the right-hand recombinase sequence comprises a sequence having at least about 97% identity to SEQ ID NO: 137 or 139. In some cases, the right-hand recombinase sequence comprises a sequence having at least about 98% identity to SEQ ID NO: 137 or 139. In some cases, the right-hand recombinase sequence comprises a sequence having at least about 99% identity to SEQ ID NO: 137 or 139. In some cases, the right-hand recombinase sequence comprises a sequence having 100% identity to SEQ ID NO: 137 or 139.

In some cases, the class 1, type I Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In some embodiments, the systems described herein comprise a nuclear localization signal (NLS) sequence. In some embodiments, the NLS is at an N-terminus of the Cas effector. In some embodiments, the NLS is at a C-terminus of the Cas effector. In some embodiments, the NLS is at an N-terminus and a C-terminus of the Cas effector

In some embodiments, the NLS comprises a sequence of any one of SEQ ID NOs: 740-755, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 740-755. In some cases, the NLS comprises a sequence having at least about 80% identity to any one of SEQ ID NOs: 740-755. In some cases, the NLS comprises a sequence having at least about 85% identity to any one of SEQ ID NOs: 740-755. In some cases, the NLS comprises a sequence having at least about 90% identity to any one of SEQ ID NOs: 740-755. In some cases, the NLS comprises a sequence having at least about 91% identity to any one of SEQ ID NOs: 740-755. In some cases, the NLS comprises a sequence having at least about 92% identity to any one of SEQ ID NOs: 740-755. In some cases, the NLS comprises a sequence having at least about 93% identity to any one of SEQ ID NOs: 740-755. In some cases, the NLS comprises a sequence having at least about 94% identity to any one of SEQ ID NOs: 740-755. In some cases, the NLS comprises a sequence having at least about 95% identity to any one of SEQ ID NOs: 740-755. In some cases, the NLS comprises a sequence having at least about 96% identity to any one of SEQ ID NOs: 740-755. In some cases, the NLS comprises a sequence having at least about 97% identity to any one of SEQ ID NOs: 740-755. In some cases, the NLS comprises a sequence having at least about 98% identity to any one of SEQ ID NOs: 740-755. In some cases, the NLS comprises a sequence having at least about 99% identity to any one of SEQ ID NOs: 740-755. In some cases, the NLS comprises a sequence having 100% identity to any one of SEQ ID NOS: 740-755.

TABLE 1
Exemplary NLS Sequences

| | | SEQ |
|---|---|---|
| | | ID |
| Source | NLS amino acid sequence | NO: |
| SV40 | PKKKRKV | 740 |
| nucleoplasmin | KRPAATKKAGQAKKKK | 741 |
| bipartite NLS | | |
| c-myc NLS | PAAKRVKLD | 742 |
| c-myc NLS | RQRRNELKRSP | 743 |
| hRNPA1 M9 NLS | NQSSNFGPMKGGNFGGRSSGPYGGGGQYFAKPRNQGGY | 744 |
| Importin-alpha | RMRIZFKNKGKDTAELRRRRVEVSVELRKAKKDEQILKRRNV | 745 |
| IBB domain | | |
| Myoma T protein | VSRKRPRP | 746 |
| Myoma T protein | PPKKARED | 747 |
| p53 | PQPKKKPL | 748 |
| mouse c-abl IV | SALIKKKKKMAP | 749 |
| influenza virus | DRLRR | 750 |
| NS1 | | |
| influenza virus | PKQKKRK | 751 |
| NS1 | | |
| Hepatitis virus | RKLKKKIKKL | 752 |
| delta antigen | | |
| mouse Mx1 | REKKKFLKRR | 753 |
| protein | | |
| human poly(ADP- | KRKGDEVDGVDEVAKKKSKK | 754 |
| ribose) polymerase | | |
| steroid hormone | RKCLQAGMNLEARKTKK | 755 |
| receptors (human) | | |
| glucocorticoid | | |

In some embodiments, the Cas effector complex further comprises a small prokaryotic ribosomal protein subunit S15. In some cases, the S15 comprises a sequence having at least about 70% identity to any one of SEQ ID NOs: 494-659. In some cases, the S15 comprises a sequence having at least about 75% identity to any one of SEQ ID NOs: 494-659. In some cases, the S15 comprises a sequence having at least about 80% identity to any one of SEQ ID NOs: 494-659. In some cases, the S15 comprises a sequence having at least about 85% identity to any one of SEQ ID NOs: 494-659. In some cases, the S15 comprises a sequence having at least about 90% identity to any one of SEQ ID NOs: 494-659. In some cases, the S15 comprises a sequence having at least about 91% identity to any one of SEQ ID NOs: 494-659. In some cases, the S15 comprises a sequence having at least about 92% identity to any one of SEQ ID NOs: 494-659. In some cases, the S15 comprises a sequence having at least about 93% identity to any one of SEQ ID NOs: 494-659. In some cases, the S15 comprises a sequence having at least about 94% identity to any one of SEQ ID NOs: 494-659. In some cases, the S15 comprises a sequence having at least about 95% identity to any one of SEQ ID NOs: 494-659. In some cases, the S15 comprises a sequence having at least about 96% identity to any one of SEQ ID NOs: 494-659. In some cases, the S15 comprises a sequence having at least about 97% identity to any one of SEQ ID NOs: 494-659. In some cases, the S15 comprises a sequence having at least about 98% identity to any one of SEQ ID NOs: 494-659. In some cases, the S15 comprises a sequence having at least about 99% identity to any one of SEQ ID NOs: 494-659. In some cases, the S15 comprises a sequence having 100% identity to any one of SEQ ID NOs: 494-659.

In some cases, the S15 comprises a sequence having at least about 70% identity to any one of SEQ ID NOs: 501, 526, 528, 536, 577, 602, 653, and 659. In some cases, the S15 comprises a sequence having at least about 75% identity to any one of SEQ ID NOs: 501, 526, 528, 536, 577, 602, 653, and 659. In some cases, the S15 comprises a sequence having at least about 80% identity to any one of SEQ ID NOs: 501, 526, 528, 536, 577, 602, 653, and 659. In some cases, the S15 comprises a sequence having at least about 85% identity to any one of SEQ ID NOs: 501, 526, 528, 536, 577, 602, 653, and 659. In some cases, the S15 comprises a sequence having at least about 90% identity to any one of SEQ ID NOs: 501, 526, 528, 536, 577, 602, 653, and 659. In some cases, the S15 comprises a sequence having at least about 91% identity to any one of SEQ ID NOs: 501, 526, 528, 536, 577, 602, 653, and 659. In some cases, the S15 comprises a sequence having at least about 92% identity to any one of SEQ ID NOs: 501, 526, 528, 536, 577, 602, 653, and 659. In some cases, the S15 comprises a sequence having at least about 93% identity to any one of SEQ ID NOs: 501, 526, 528, 536, 577, 602, 653, and 659. In some cases, the S15 comprises a sequence having at least about 94% identity to any one of SEQ ID NOs: 501, 526, 528, 536, 577, 602, 653, and 659. In some cases, the S15 comprises a sequence having at least about 95% identity to any one of SEQ ID NOS: 501, 526, 528, 536, 577, 602, 653, and 659. In some cases, the S15 comprises a sequence having at least about 96% identity to any one of SEQ ID NOs: 501, 526, 528, 536, 577, 602, 653, and 659. In some cases, the S15 comprises a sequence having at least about 97% identity to any one of SEQ ID NOs: 501, 526, 528, 536, 577, 602, 653, and 659. In some cases, the S15 comprises a sequence having at least about 98% identity to any one of SEQ ID NOs: 501, 526, 528, 536, 577, 602, 653, and 659. In some cases, the S15 comprises a sequence having at least about 99% identity to any one of SEQ ID NOs: 501, 526, 528, 536, 577, 602, 653, and 659. In some cases, the S15 comprises a sequence having 100% identity to any one of SEQ ID NOs: 501, 526, 528, 536, 577, 602, 653, and 659.

### Fusion Proteins

Described herein, in some embodiments, are systems for transposing a cargo nucleotide sequence into a target nucleic acid site comprising a fusion protein or a nucleic acid encoding the fusion protein. In some embodiments, the fusion protein or a nucleic acid encoding the fusion protein comprises a Cas effector, a small prokaryotic ribosomal protein subunit S15, a transposase, a gRNA, or combinations thereof. In some embodiments, the fusion protein comprises one or more transposases.

In some embodiments, an NLS is fused to the Cas effector. In some embodiments, the NLS is fused at an N-terminus of the Cas effector. In some embodiments, the NLS is fused at a C-terminus of the Cas effector. In some embodiments, the NLS is fused at an N-terminus and a C-terminus of the Cas effector.

In some embodiments, the NLS comprises a sequence of any one of SEQ ID NOs: 740-755, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 740-755. In some cases, the NLS comprises a sequence having at least about 80% identity to any one of SEQ ID NOs: 740-755. In some cases, the NLS comprises a sequence having at least about 85% identity to any one of SEQ ID NOs: 740-755. In some cases, the NLS comprises a sequence having at least about 90% identity to any one of SEQ ID NOs: 740-755. In some cases, the NLS comprises a sequence having at least about 91% identity to any one of SEQ ID NOs: 740-755. In some cases, the NLS comprises a sequence having at least about 92% identity to any one of SEQ ID NOs: 740-755. In some cases, the NLS comprises a sequence having at least about 93% identity to any one of SEQ ID NOs: 740-755. In some cases, the NLS comprises a sequence having at least about 94% identity to any one of SEQ ID NOs: 740-755. In some cases, the NLS comprises a sequence having at least about 95% identity to any one of SEQ ID NOs: 740-755. In some cases, the NLS comprises a sequence having at least about 96% identity to any one of SEQ ID NOs: 740-755. In some cases, the NLS comprises a sequence having at least about 97% identity to any one of SEQ ID NOs: 740-755. In some cases, the NLS comprises a sequence having at least about 98% identity to any one of SEQ ID NOs: 740-755. In some cases, the NLS comprises a sequence having at least about 99% identity to any one of SEQ ID NOs: 740-755. In some cases, the NLS comprises a sequence having 100% identity to any one of SEQ ID NOs: 740-755.

In some embodiments, the nucleic acid comprises a fusion of S15 and a nuclear localization sequence (NLS). In some embodiments, the NLS is fused at an N-terminus of S15.

In some embodiments, the S15 protein further comprises a cleavable peptide. In some embodiments, the peptide is a 2A peptide.

In some embodiments, the S15 fusion protein comprises a sequence having at least about 70% sequence identity to any one of any one of SEQ ID NOs: 494-659. In some embodiments, the S15 fusion protein has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of any one of SEQ ID NOs: 494-659. In some cases, the S15 comprises a sequence having at least about 70% identity to any one of SEQ ID NOs: 494-659. In some cases, the S15 comprises a sequence having at least about 75% identity to any one of SEQ ID NOs: 494-659. In some cases, the S15 comprises a sequence having at least about 80% identity to any one of SEQ ID NOs: 494-659. In some cases, the S15 comprises a sequence having at least about 85% identity to any one of SEQ ID NOs: 494-659. In some cases, the S15 comprises a sequence having at least about 90% identity to any one of SEQ ID NOs: 494-659. In some cases, the S15 comprises a sequence having at least about 91% identity to any one of SEQ ID NOs: 494-659. In some cases, the S15 comprises a sequence having at least about 92% identity to any one of SEQ ID NOs: 494-659. In some cases, the S15 comprises a sequence having at least about 93% identity to any one of SEQ ID NOs: 494-659. In some cases, the S15 comprises a sequence having at least about 94% identity to any one of SEQ ID NOs: 494-659. In some cases, the S15 comprises a sequence having at least about 95% identity to any one of SEQ ID NOs: 494-659. In some cases, the S15 comprises a sequence having at least about 96% identity to any one of SEQ ID NOs: 494-659. In some cases, the S15 comprises a sequence having at least about 97% identity to any one of SEQ ID NOs: 494-659. In some cases, the S15 comprises a sequence having at least about 98% identity to any one of SEQ ID NOs: 494-659. In some cases, the S15 comprises a sequence having at least about 99% identity to any one of SEQ ID NOs: 494-659. In some cases, the S15 comprises a sequence having 100% identity to any one of SEQ ID NOs: 494-659.

In some embodiments, the S15 fusion protein comprises a sequence having at least about 70% sequence identity to any one of SEQ ID NOs: 501, 526, 528, 536, 577, 602, 653, and 659. In some embodiments, the S15 fusion protein has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 501, 526, 528, 536, 577, 602, 653, and 659. In some cases, the S15 comprises a sequence having at least about 70% identity to any one of SEQ ID NOs: 501, 526, 528, 536, 577, 602, 653, and 659. In some cases, the S15 comprises a sequence having at least about 75% identity to any one of SEQ ID NOs: 501, 526, 528, 536, 577, 602, 653, and 659. In some cases, the S15 comprises a sequence having at least about 80% identity to any one of SEQ ID NOs: 501, 526, 528, 536, 577, 602, 653, and 659. In some cases, the S15 comprises a sequence having at least about 85% identity to any one of SEQ ID NOs: 501, 526, 528, 536, 577, 602, 653, and 659. In some cases, the S15 comprises a sequence having at least about 90% identity to any one of SEQ ID NOs: 501, 526, 528, 536, 577, 602, 653, and 659. In some cases, the S15 comprises a sequence having at least about 91% identity to any one of SEQ ID NOs: 501, 526, 528, 536, 577, 602, 653, and 659. In some cases, the S15 comprises a sequence having at least about 92% identity to any one of SEQ ID NOs: 501, 526, 528, 536, 577, 602, 653, and 659. In some cases, the S15 comprises a sequence having at least about 93% identity to any one of SEQ ID NOs: 501, 526, 528, 536, 577, 602, 653, and 659. In some cases, the S15 comprises a sequence having at least about 94% identity to any one of SEQ ID NOs: 501, 526, 528, 536, 577, 602, 653, and 659. In some cases, the S15 comprises a sequence having at least about 95% identity to any one of SEQ ID NOS: 501, 526, 528, 536, 577, 602, 653, and 659. In some cases, the S15 comprises a sequence having at least about 96% identity to any one of SEQ ID NOs: 501, 526, 528, 536, 577, 602, 653, and 659. In some cases, the S15 comprises a sequence having at least about 97% identity to any one of SEQ ID NOs: 501, 526, 528, 536, 577, 602, 653, and 659. In some cases, the S15 comprises a sequence having at least about 98% identity to any one of SEQ ID NOs: 501, 526, 528, 536, 577, 602, 653, and 659. In some cases, the S15 comprises a sequence having at least about 99% identity to any one of SEQ ID NOs: 501, 526, 528, 536, 577, 602, 653, and 659. In some cases, the S15 comprises a sequence having 100% identity to any one of SEQ ID NOs: 501, 526, 528, 536, 577, 602, 653, and 659.

In some embodiments, an NLS is fused to the transposase. In some embodiments, the NLS is fused at an N-terminus of the transposase. In some embodiments, the NLS is fused at a C-terminus of the transposase. In some embodiments, the NLS is fused at an N-terminus and a C-terminus of the transposase. In some embodiments, the NLS comprises a sequence of any one of SEQ ID NOs: 740-755, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 740-755. In some embodiments, the transposase is TnsB, TasC, or TniQ. In some embodiments, the transposase is TnsB. In some embodiments, the transposase is TnsC. In some embodiments, the transposase is TniQ.

In some embodiments, the class 2, type V effector, the small prokaryotic ribosomal protein subunit S15, the transposase, the single gRNA, or a fusion protein or gene editing system comprising any combination thereof, comprises a tag. In some embodiments, the tag is an affinity tag. Exemplary affinity tags include, but are not limited to, a His-tag, a Flag tag, a Myc-tag, an MBP-tag, and a GST-tag.

In some embodiments, the class 2, type V effector, the small prokaryotic ribosomal protein subunit S15, the transposase, the single gRNA, or a fusion protein or gene editing system comprising any combination thereof, comprises a protease cleavage site. Exemplary protease cleavage sites include, but are not limited to, a TEV site, a C3 site, a Factor Xa site, and an Enterokinase site.

### Cells

Described herein, in certain embodiments, are cells comprising the systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaca cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof.

### Delivery and Vectors

Disclosed herein, in some embodiments, are nucleic acid sequences encoding a CAST system described herein comprising a class 2, type V effector, a small prokaryotic ribosomal protein subunit S15, a transposase, a gRNA, a fusion protein or a gene editing system disclosed herein.

In some embodiments, the nucleic acid encoding the CAST system described herein is a DNA, for example a linear DNA, a plasmid DNA, or a minicircle DNA. In some embodiments, the nucleic acid encoding the CAST system described herein is an RNA, for example a mRNA.

In some embodiments, the nucleic acid encoding the CAST system described herein is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3XFLAG, pSF-CMV-NEO-COOH-3XFLAG, pSF-CMV—PURO-NH2-GST-TEV, pSF-OXB20-COOH-TEV-FLAG (R)-6His, pCEP4 pDEST27, pSF-CMV-Ub-KrYFP, pSF-CMV-FMDV-daGFP, pEFla-mCherry-N1 vector, pEFla-tdTomato vector, pSF-CMV-FMDV-Hygro, pSF-CMV-PGK-Puro, pMCP-tag (m), pSF-CMV—PURO-NH2-CMYC, pSF-OXB20-BctaGal,pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EFla, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter. In some embodiments, the promoter is encoded by a sequence of any one of SEQ ID NOs: 190-191, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity of any one of SEQ ID NOs: 190-191.
In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.
In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV—PHP—B, AAV-PHP-EB, AAV-2.5, AAV-21YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.
In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV—PHP—B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.
In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.
In some embodiments, the nucleic acid encoding the MG64 system delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the fusion protein or genome editing system is introduced into the cell in any suitable way, cither stably or transiently. In some embodiments, a fusion protein or genome editing system is transfected into the cell. In some embodiments, the cell is transduced or transfected with a nucleic acid construct that encodes a fusion protein or genome editing system. For example, a cell is transduced (e.g., with a virus encoding a fusion protein or genome editing system), or transfected (e.g., with a plasmid encoding a fusion protein or genome editing system) with a nucleic acid that encodes a fusion protein or genome editing system, or the translated fusion protein or genome editing system. In some embodiments, the transduction is a stable or transient transduction. In some embodiments, cells expressing a fusion protein or genome editing system or containing a fusion protein or genome editing system are transduced or transfected with one or more gRNA molecules, for example when the fusion protein or genome editing system comprises a CRISPR nuclease. In some embodiments, a plasmid expressing a fusion protein or genome editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is part of a liposome or a nanoparticle that specifically targets a host cell.

Additional methods for the delivery of nucleic acids to cells are known to those skilled in the art. Sec, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Methods for Transposition

The present disclosure provides methods for transposing a cargo nucleotide sequence into a target nucleic acid site. In some embodiments, the method comprises expressing a system described herein within a cell or introducing a system described herein to a cell. In some embodiments, the method comprises contacting a cell with a system described herein.

In some embodiments, the present disclosure provides for a method for transposing a cargo nucleotide sequence into a target nucleic acid site, comprising contacting a double-stranded nucleic acid comprising a cargo nucleotide sequence with a Cas effector complex. In some embodiments, Cas effector complex comprises a class 2, type II Cas effector, a class 2, type V, or a class I, type I-F, and at least one engineered guide polynucleotide configured to hybridize to the target nucleic acid site. In some embodiments, the method comprises contacting the double-stranded nucleic acid comprising the cargo nucleotide sequence with a recombinase or transposase complex configured to recruit the cargo nucleotide to the target nucleic acid site. In some embodiments, the method comprises contacting the double-stranded nucleic acid comprising the cargo nucleotide sequence with a target nucleic acid comprising the target nucleic acid site.

In some cases, the cargo nucleotide sequence is flanked by a left-hand transposase recognition sequence. In some cases, the cargo nucleotide sequence is flanked by a right-hand transposase recognition sequence. In some cases, the cargo nucleotide sequence is flanked by a left-hand transposase recognition sequence and a right-hand transposase recognition sequence. In some cases, the Cas effector complex further comprises a PAM sequence compatible with the Cas effector complex adjacent to the target nucleic acid site. In some cases, the PAM sequence is located 3′ of the target nucleic acid site. In some cases, the PAM sequence is located 5′ of the target nucleic acid site.

### Uses

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance in bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Kits

In some embodiments, this disclosure provides kits comprising one or more nucleic acid constructs encoding the various components of the genome editing system described herein, e.g., comprising a nucleotide sequence encoding the components of the genome editing system capable of modifying a target DNA sequence. In some embodiments, the nucleotide sequence comprises a heterologous promoter that drives expression of the RNA genome editing system components.

In some embodiments, the class 2, type V effector, the small prokaryotic ribosomal protein subunit S15, the transposase, the single gRNA, or a fusion protein or gene editing system comprising any combination thereof disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1—(General Protocol) PAM Sequence Identification/Confirmation for Systems Described Herein

Putative endonucleases were expressed in an E. coli lysate-based expression system. PAM sequences were determined by sequencing plasmids containing randomly-generated potential PAM sequences that could be cleaved by the putative nucleases. In this system, an E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence in the in vitro expression system followed by CRISPR array processing provided active in vitro CRISPR nuclease complexes.

A library of target plasmids containing a spacer sequence matching that in the minimal array preceded by 8N mixed bases (potential PAM sequences) was incubated with the output of the in vitro expression reaction. After 1-3 hr, the reaction was stopped and the DNA was recovered via a DNA clean-up kit. Adapter sequences were blunt-end ligated to DNA with active PAM sequences that were cleaved by the endonuclease, whereas DNA that was not cleaved is inaccessible for ligation. DNA segments comprising active PAM sequences were then amplified by PCR with primers specific to the library and the adapter sequence. The PCR amplification products were resolved on a gel to identify amplicons that correspond to cleavage events. The amplified segments of the cleavage reaction were also used as templates for preparation of an NGS library or as a substrate for Sanger sequencing. Sequencing this resulting library, which is a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template is omitted.

For endonucleases which are binding-competent but nuclease deficient, the PAM was determined via a modification of the above procedure. After expression in the in vitro expression system, the sgRNA or crRNA and PAM library were added. Upon binding of the effector in a sgRNA-dependent manner to the spacer sequence, the spacer sequence was sequestered within the effector protein. The appropriate restriction enzyme that targets within the spacer sequence was added and all unprotected plasmids within the library were cleaved. The uncleaved (endonuclease-bound) members of the library which contain the PAM were identified by PCR and subsequent NGS library preparation of the band.

### Example 2—In Vitro Targeted Integrase Activity

Integrase activity was assayed with a previously identified PAM but may be conducted with a PAM library substrate instead, with reduced efficiency. One arrangement of components for in vitro testing involved three plasmids other than that containing the donor sequence: (1) an expression plasmid with effector (or effectors) under a T7 promoter; (2) an expression plasmid with integrase genes under a T7 promoter; a sgRNA or crRNA and tracrRNA; (3) a target plasmid which contained the spacer site and appropriate PAM; and (4) a donor plasmid which contained the required left end (LE) and right end (RE) DNA sequences for transposition around a cargo gene (e.g., a selection marker such as a Tet resistance gene). Using an in vitro transcription/translation system (e.g., E. coli lysate- or reticulocyte lysate-based system), the effector and integrase genes were expressed. After expression, the RNA, target DNA, and donor DNA were added and incubated to allow for transposition to occur. Transposition was detected via PCR across the junction of the integrase site, with one primer on the target DNA and one primer on the donor DNA. The resulting PCR product was sequenced via NGS to determine the exact insertion topology relative to the sgRNA/crRNA targeted site. The primers were located downstream such that a variety of insertion sites can be accommodated and detected. Primers were designed such that integration is detected in either orientation of cargo or on either side of the spacer, as the integration direction was also not previously documented.

Integration efficiency was measured via quantitative PCR (qPCR) measurements of the experimental output of target DNA with integrated cargo, normalized to the amount of unmodified target DNA also measured via qPCR.

This assay may be conducted with purified protein components rather than from lysate-based expression. In this case the proteins were expressed in an E. coli protease deficient B strain under a T7 inducible promoter, the cells were lysed using sonication, and the His-tagged protein of interest was purified using Ni-NTA affinity chromatography on an FPLC. Purity was determined using densitometry of the protein bands resolved on SDS-PAGE and coomassie stained acrylamide gels. The protein was desalted in storage buffer composed of 50 mM Tris-HCl, 300 mM NaCl, 1 mM TCEP, 5% glycerol; pH 7.5 (or other buffers as determined for maximum stability) and stored at −80° C. After purification the effector(s) and integrase(s) were added to the sgRNA, target DNA, and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol, (final pH 7.5) supplemented with 15 mM Mg (OAc)₂.

### Example 3—Predicted RNA Folding

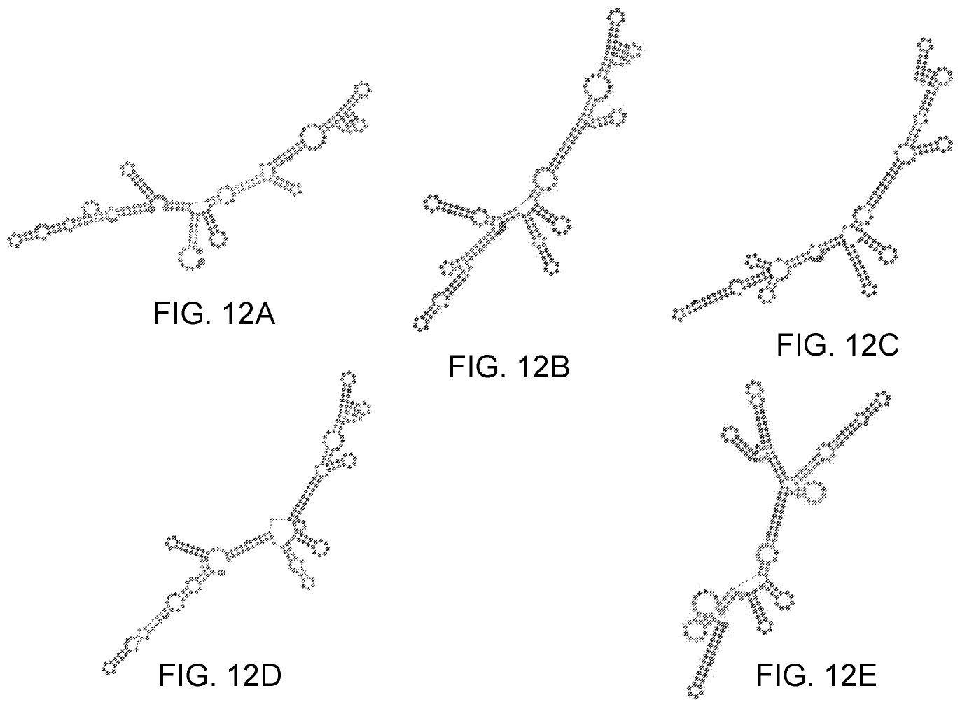

Predicted RNA folding of the active single RNA sequence was computed at 37° using the method of Andronescu 2007. All hairpin-loop secondary structures were singly deleted from the structure and iteratively compiled into a smaller single guide. In a second approach, the tracrRNA of MG64-1 was aligned to documented type V-k tracrRNA, and areas of unique insertions were mutated out of the single guide and minimized by 57 bases. FIG. 12A depicts the predicted structure of MG64-2 sgRNA (SEQ ID NO: 202). FIG. 12B depicts the predicted structure of MG64-4 sgRNA (SEQ ID NO: 203). FIG. 12C depicts the predicted structure of MG64-6 sgRNA (SEQ ID NO: 201). FIG. 12D depicts the predicted structure of MG64-7 sgRNA (SEQ ID NO: 204). FIG. 12E depicts the predicted structure of MG108-1 sgRNA (SEQ ID NO: 206). The shading of the bases corresponds to the probability of base pairing of that base.

### Example 4—Transposon End Verification Via Gel Shift

The transposon ends were tested for TnsB binding via an electrophoretic mobility shift assay (EMSA). In this case the potential LE or RE was synthesized as a DNA fragment (100-500 bp) and end-labeled with FAM via PCR with FAM-labeled primers. The TnsB protein was synthesized in an in vitro transcription/translation system. After synthesis, 1 μL of TnsB protein was added to 50 nM of the labeled RE or LE in a 10 μL reaction in binding buffer (20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding was incubated at 30° for 40 minutes, then 2 uL of 6× loading buffer (60 mM KCl, 10 mM Tris pH 7.6, 50% glycerol) was added. The binding reaction was separated on a 5% TBE gel and visualized. Shifts of the LE or RE in the presence of TnsB were attributed to successful binding and were indicative of transposase activity.

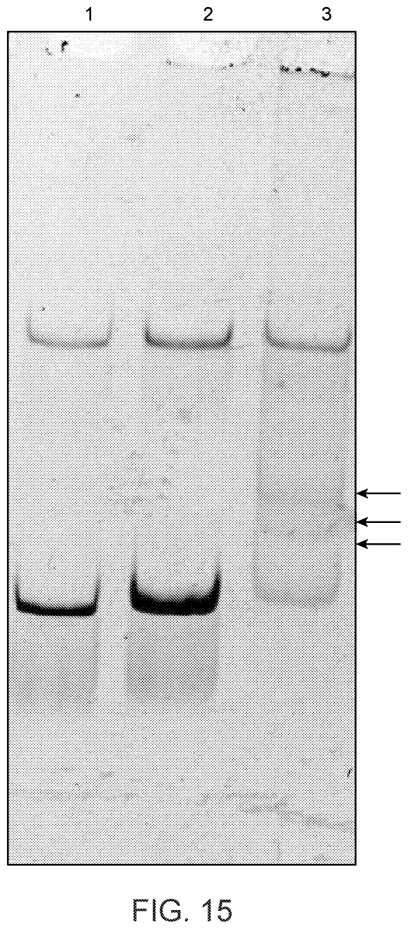

FIG. 15 shows an example of this experiment, where the RE DNA sequence for MG64-2 (e.g., SEQ ID NO: 155) was end-labeled with FAM by the above procedure and incubated with the corresponding MG64-2 TnsB-like component (e.g., SEQ ID NO: 23). Upshift of the labeled band in Lane 3 indicates binding of the RE sequence by TnsB, indicating it contains an active RE transposition sequence.

### Example 5—Integrase Activity in E. coli (Prophetic)

As E. coli lacks the capacity to efficiently repair genomic double-stranded DNA breaks, transformation of E. coli by agents able to cause double-stranded breaks in the E. coli genome causes cell death. Exploiting this phenomenon, endonuclease or effector-assisted integrase activity is tested in E. coli by recombinantly expressing either the endonuclease or effector-assisted integrase and a guide RNA (determined e.g., as in Example 3) in a target strain with spacer/target and PAM sequences integrated into its genomic DNA.

Engineered strains are then transformed with a plasmid containing the nuclease or effector with single guide RNA, a plasmid expressing the integrase and accessory genes, and a plasmid containing a temperature sensitive origin of replication with a selectable marker flanked by left end (LE) and right end (RE) transposon motifs for integration. Transformants induced for expression of these genes are then screened for transfer of the marker to the genomic target by selection at restrictive temperature for plasmid replication and the marker integration in the genome is confirmed by PCR.

Off-target integrations are screened using an unbiased approach. In brief, purified gDNA is fragmented with Tn5 integrase or shearing, and DNA of interest is then PCR amplified using primers specific to a ligated adaptor and the selectable marker. The amplicons are then prepared for NGS sequencing. Analysis of the resulting sequences is trimmed of the transposon sequences and flanking sequences are mapped to the genome to determine insertion position, and off target insertion rates are determined.

### Example 6—Colony PCR Screen of Transposase Activity (Prophetic)

For testing of nuclease or effector assisted integrase activity in bacterial cells, strain MGB0032 is constructed from BL21 (DE3) E. coli cells which are engineered to contain the target and corresponding PAM sequence specific to MG64_1. MGB0032 E. coli cells are then transformed with pJL56 (plasmid that expresses the MG64_1 effector and helper suite, ampicillin resistant) and pTCM 64_1 sg, a chloramphenicol-resistant plasmid that expresses the single guide RNA sequence for the engineered target of interest driven by a T7 promoter.

An MGB0032 culture containing both plasmids is then grown to a saturation, diluted at least 1:10 into growth culture with appropriate antibiotics, and incubated at 37° C. until OD of approximately 1. Cells from this growth stage are made electrocompetent and transformed with streamlined 64_1 pDonor, a plasmid bearing a tetracycline resistance marker flanked by left end (LE) and right end (RE) transposon motifs for integration. Electroporated cells are then recovered for 2 hours on LB medium in the presence or absence of IPTG at a concentration of 100 μM before being plated on LB-agar-ampicillin-chloramphenicol-tetracycline and incubated 4 days at 37° C. Sterile toothpicks are used to sample each resultant CFU, which is mixed into water. To this solution is added Q5 High Fidelity PCR mastermix and primers LA155 (5′-GCTCTTCCGATCTNNNNNGATGAGCGCATTGTTAGATTTCAT-3′ (SEQ ID NO; 756)) and oJL50 (5′-AAACCGACATCGCAGGCTTC-3′ (SEQ ID NO: 757)). These primers flank the predicted insertion junction. The predicted product size is 609 bp. DNA amplified PCR product is visualized on a 2% agarose gel. Sanger sequencing of PCR products confirms the transposition event.

### Example 7—in Cell Expression/In Vitro Assay (Prophetic)

To test the functionality of the NLS constructs in a physiologically relevant environment, constructs cloned with active NLS-tagged CAST components are integrated into K562 cells using lentiviral transduction. Briefly, constructs cloned into lentiviral transfer plasmids are transfected into 293T cells with envelope and packaging plasmids, and virus containing supernatant is harvested from the media after 72 hr incubation. Media containing virus is then incubated with K562 cell lines with 8 μg/mL of polybrene for 72 hrs, and transfected cells are then selected for integration in bulk using Puromycin at 1 μg/mL for 4 days. Cell lines undergoing selection are harvested at the end of 4 days, and differentially lysed for nuclear and cytoplasmic fractions. Subsequent fractions are then tested for transposition capability with a complementary set of in vitro expressed components.

10 million cells are centrifuged and washed once with 1×PBS pH7.4. Supernatant wash is aspirated completely to the cell pellet, and flash frozen at −80° C. for 16 hrs. After thawing on ice, cell pellet size is measured by mass, and appropriate extraction volumes of cell fractionation and nuclear extraction reagent is used to natively extract proteins in cell fractions. Briefly, cytoplasmic extraction reagent is used at 1:10 mass of cells to volume of extraction reagent. Cell suspension is mixed by vortexing and lysed with non-ionic detergent. Cells are then centrifuged at 16,000×g at 4° C. for 5 minutes. Cytoplasmic extraction supernatant is then decanted and saved for in vitro testing. Nuclear extraction reagent is then added 1:2 original cell mass to nuclear extraction reagent and incubated on ice for 1 hr on ice with intermittent vortexing. Nuclear suspension is then centrifuged at 16,000×g for 10 minutes at 4° C. and supernatant nuclear extract is decanted and tested for in vitro transposition activity. Using 4 μL of each cell and nuclear extract for each condition, the in vitro transposition reaction is performed with a complementary set of in vitro expressed proteins, donor DNA, pTarget, and buffer. Evidence of transposition activity is assayed by PCR amplification of donor-target junctions.

### Example 8—Activity in Mammalian Cells (Prophetic)

To show targeting and cleavage activity in mammalian cells, nuclear localization sequences are fused to the C terminus of each of the nuclease or effector proteins and integrase proteins and the fusion proteins are purified. A single guide RNA targeting a genomic locus of interest is synthesized and incubated with the nuclease/effector protein to form a ribonucleoprotein complex. Cells are transfected with a plasmid containing a selectable neomycin resistance marker (NeoR) or a fluorescent marker flanked by the left end (LE) and right end (RE) motifs, recovered for 4-6 hours, and subsequently electroporated with nuclease RNP and integrase proteins. Integration of a plasmid into the genome is quantified by counting G418-resistant colonies or fluorescence activated cell cytometry. Genomic DNA is extracted 72 hours after electroporation and used for the preparation of an NGS-library. Off target frequency is assayed by fragmenting the genome and preparing amplicons of the transposon marker and flanking DNA for NGS library preparation. At least 40 different target sites are chosen for testing each targeting system's activity.

### Example 9—Activity of Targeted Nuclease

In situ expression and protein sequence analyses suggest that some RNA guided effectors are active nucleases. They contain predicted endonuclease-associated domains (matching RuvC and HNH_endonuclease domains) and predicted HNH and RuvC catalytic residues (see, e.g., FIG. 4A, which shows predicted catalytic residues of the MG36-5 effector).

Candidate activity is tested with engineered single guide RNA sequences using the in vitro expression system system and in vitro transcribed RNA. Active proteins are identified as those that successfully cleave the library to yield a band around 170 bp in agarose gel electrophoresis

### Example 10—Identification of Transposons

Transposons are predicted to be active when they contain one or more protein sequences with integrase and/or integrase function between the left and right ends of the transposon. An example Tn7 transposon generally comprises a catalytic integrase TnsB, but may also contain TnsA, TnsC, TnsD, TnsE, TniQ, and/or other integrases or integrases. The transposon ends comprise predicted integrase binding sites, which contain direct and/or inverted repeats of 15 bp to 150 bp in length flanking the integrase proteins and other ‘cargo’ genes. Protein sequence analysis indicated that the integrases contain integrase domains, integrase domains and/or integrase catalytic residues, suggesting that they are active (e.g., FIG. 4A, which shows a locus diagram for an example MG36-5 effector-based CAST system containing TnsB elements; and FIG. 5A, which shows a locus diagram for an example MG39-1 effector-based CAST system containing TnsA, TnsB, TnsC, and TniQ elements).

### Example 11—Identification of CRISPR-Associated Transposons

Putative CRISPR-associated transposons (CAST) contain a DNA and/or RNA targeting CRISPR effector and proteins with predicted integrase function in the vicinity of a CRISPR array. In some systems, the effector is predicted to have nuclease activity based on the presence of endonuclease-associated catalytic domains and/or catalytic residues (e.g., FIG. 4A, which shows predicted catalytic residues of the MG36-5 effector in the context of a CAST system locus containing TnsB elements). The integrases were predicted to be associated with the active nucleases when the CRISPR loci (CRISPR nuclease and array) and the integrase proteins are located between the predicted transposon left and right ends (e.g., FIGS. 4B-4C). In this case, the effector was predicted to direct DNA integration to specific genomic locations based on a guide RNA.

In some systems, the effector was predicted to have homology with documented CRISPR effector proteins, but to be inactive based on the absence of endonuclease domains and/or catalytic residues (FIG. 5A). The integrases are predicted to be associated with the effector when the CRISPR loci (inactive CRISPR nuclease and array) and the integrase proteins are located within the predicted transposon left and right ends (FIGS. 5A-5B).

### Example 12—CAST Discovery

CRISPR-associated transposons (CAST) are systems that comprise a transposon that has evolved to interact with a CRISPR system to promote targeted integration of DNA cargo.

CASTs are genomic sequences encoding one or more protein sequences involved in DNA transposition within the signature left and right ends of the transposon. An example Tn7 transposon, generally comprises a catalytic transposase TnsB, but may also contain a catalytic transposase TnsA, a loader protein TnsC or TniB, and target recognition proteins TnsD, TnsE, TniQ, and/or other transposon-associated components. The transposon ends comprise predicted transposase binding sites, which contain direct and/or inverted repeats of 15 bp to 150 bp in length flanking the transposon machinery and other ‘cargo’ genes.

In addition, CASTs also encode a DNA and/or RNA targeting CRISPR nuclease or effector in the vicinity of a CRISPR array. In some systems, the effector is predicted to be an active nuclease based on the presence of endonuclease-associated catalytic domains and/or catalytic residues. In some systems, the effector was predicted to have sequence similarity with documented CRISPR effector proteins, but to be inactive based on the absence of endonuclease domains and/or catalytic residues. The transposons are predicted to be associated with the effector when the CRISPR locus and the transposon-associated proteins are located within the predicted transposon left and right ends. In this case, the effector is predicted to direct DNA integration to specific genomic locations based on a guide RNA.

### Example 13a—Cas12k CAST

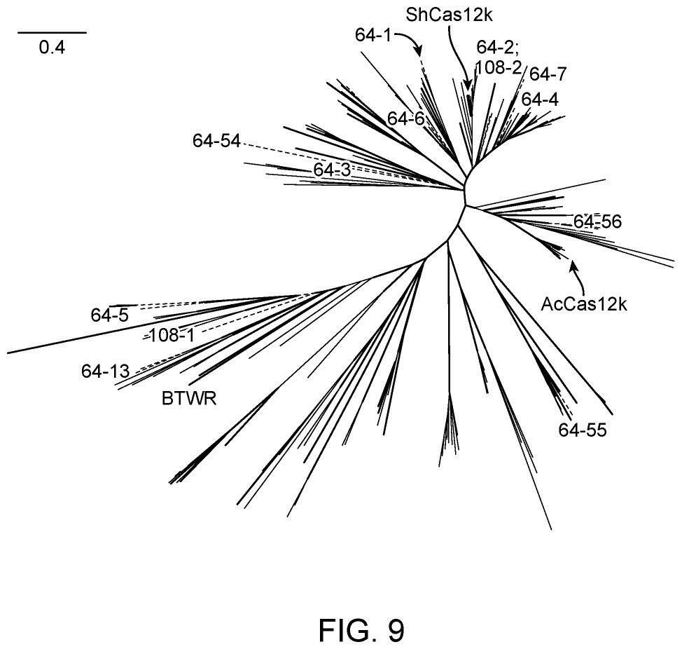

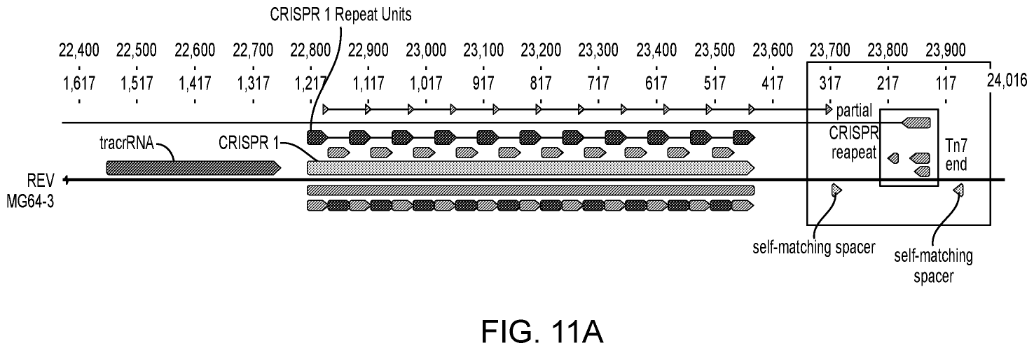

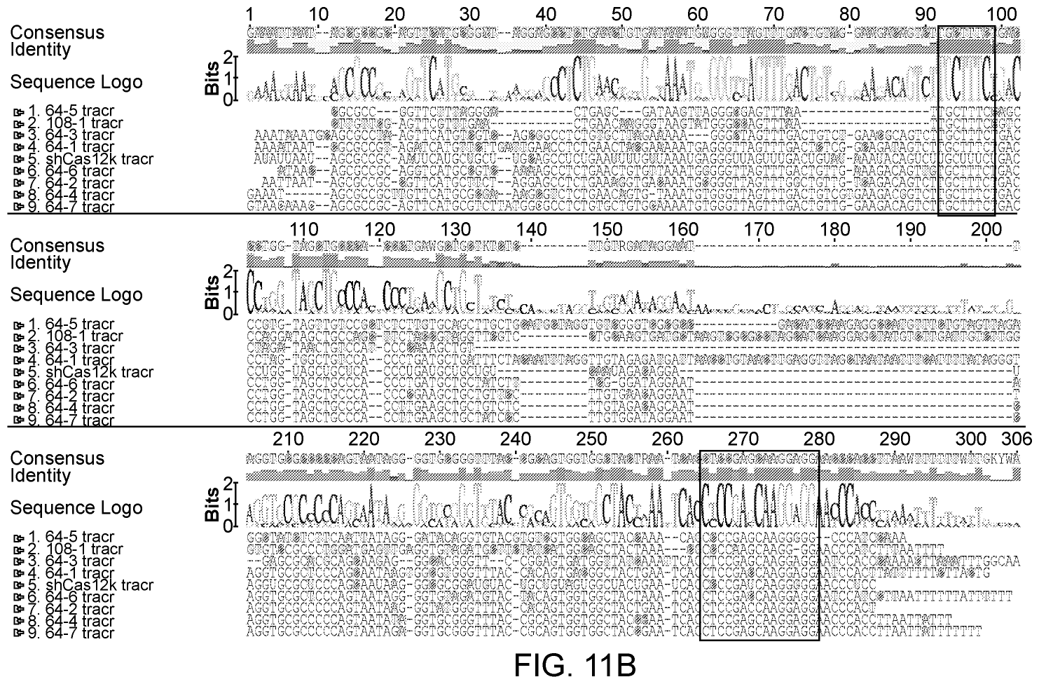

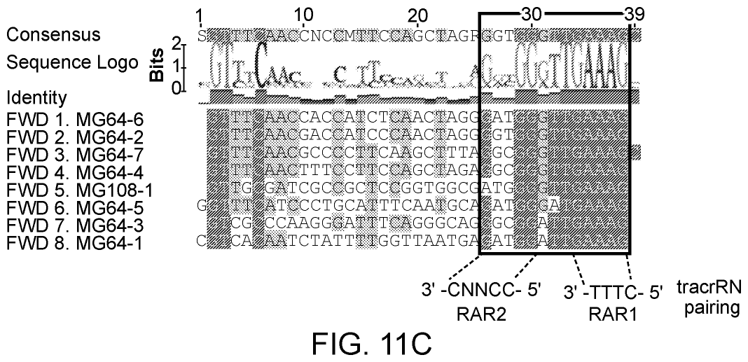

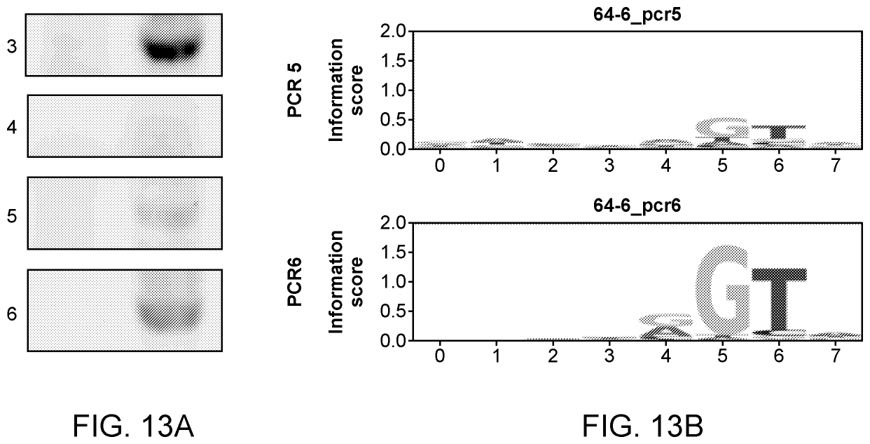

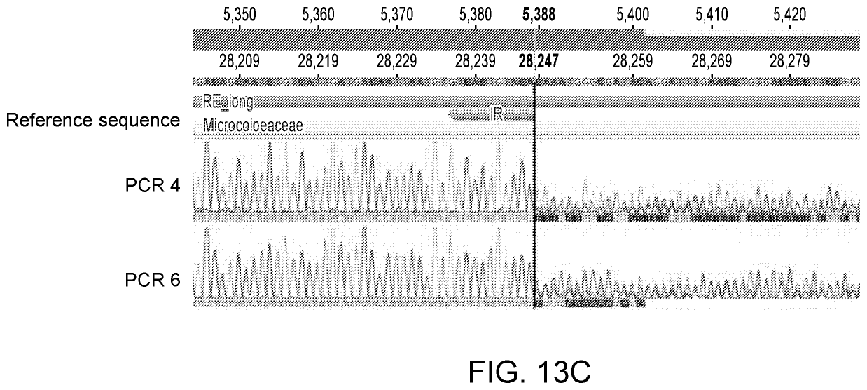

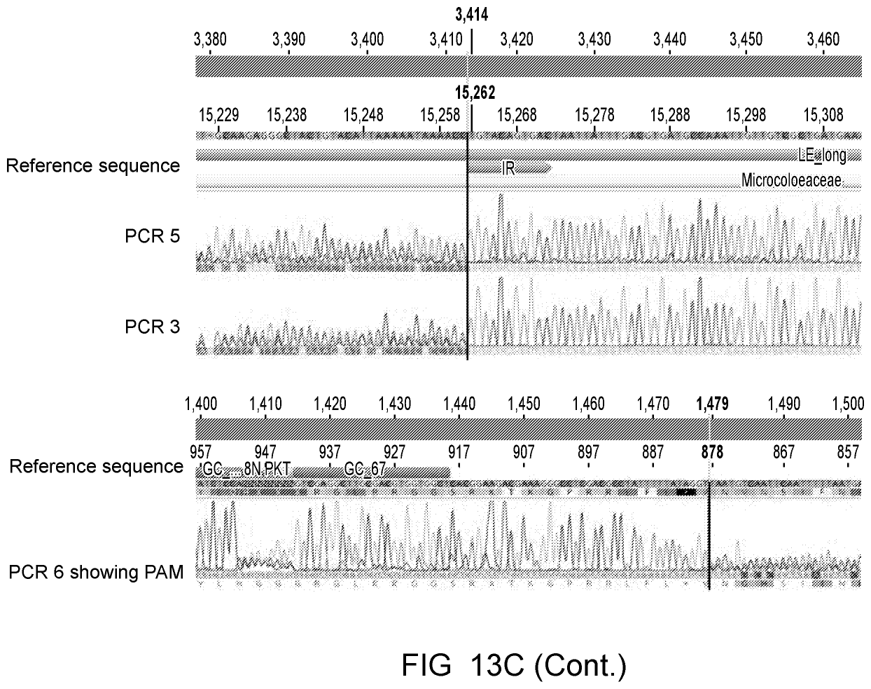

Cas12k CAST systems encode a nuclease-defective CRISPR Cas12k effector, a CRISPR array, a tracrRNA, and Tn7-like transposition proteins (see, e.g., FIG. 8, which shows a locus organization diagram for MG108-1 CAST system containing Cas12k). Cas12k effectors are phylogenetically diverse and features that confirm their association with CASTs have been confirmed for several (see, e.g., FIG. 9, which shows how MG64-1, MG64-2, MG64-3, MG 64-5, MG64-6, MG64-7, MG64-13, MG64-54, MG64-56, MG108-1, and MG108-2 effectors are part of this group). One such characteristic feature was transposon ends identified in the context of the MG64-3 CRISPR locus; the transposon left end was identified downstream from the MG64-3 CRISPR locus, as shown by terminal inverted repeats and self-matching spacer sequences (FIG. 11A). Another such characteristic that was identified includes Cas12k CAST CRISPR repeats (crRNA) which contain a conserved motif 5′-GNNGGNNTGAAAG-3′ (scc e.g., MG64-2, MG64-4, MG64-5, MG64-6, MG64-7, and MG108-1 and FIG. 11B). Short repeat-antirepeats (RAR) within the crRNA motif aligned with different regions of the tracrRNA, and RAR motifs appeared to define the start and end of the tracrRNA. FIG. 13C shows presence of these RAR motifs in e.g., MG64-2, MG64-4, MG64-5, MG64-6, MG64-7, and MG108-1 families.

### Example 13b—Class 1 Type I-F CAST

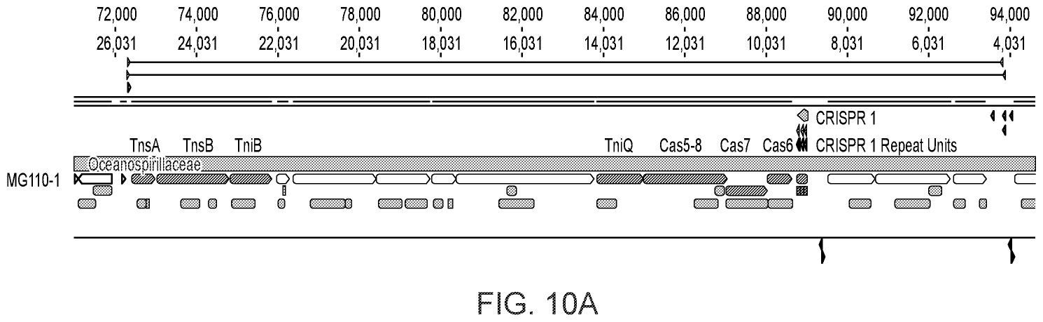

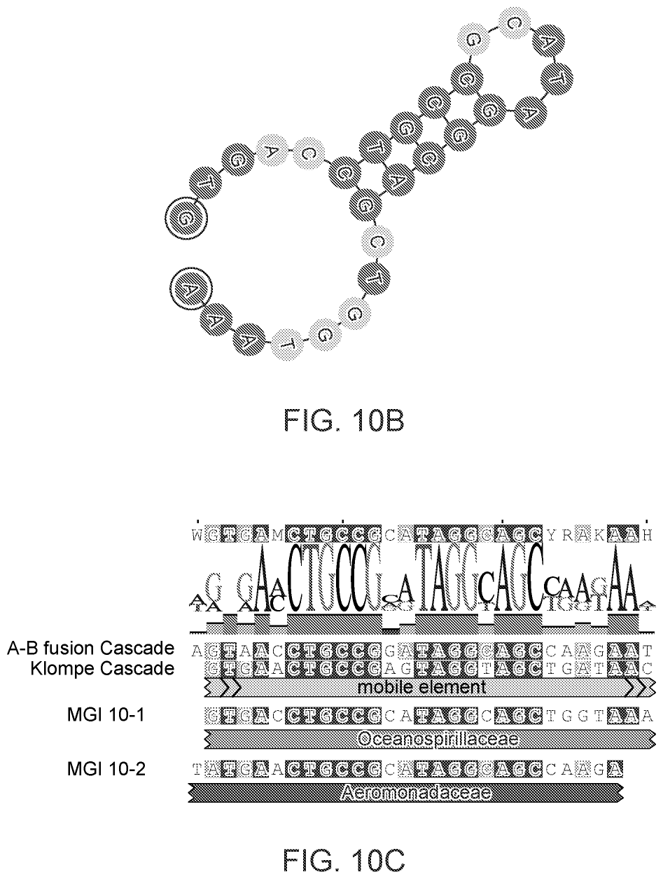

Some CASTs encode nuclease-defective CRISPR Type I-F Cascade effector proteins, a CRISPR array, and Tn7-like transposition proteins (see, e.g., FIG. 10A, which shows a locus organization diagram of a MG110-1 effector-based Type I-F CAST system). Type I-F Cascade CAST were predicted to function with a single guide RNA encoded by the crRNA, which contains a conserved motif 5′-CTGCCGNNTAGGNAGC-3′ (SEQ ID NO: 758) likely involved in formation of a stem-loop structure (see, e.g., FIGS. 10B-10C, which show an alignment of this feature in MG110-1 and MG110-2 family crRNAs SEQ ID NOs: 207 and 208). Based in part on its having these same features, the MG110-2 effector-containing and family was also identified as a Type I-F CAST system.

### Example 14—Transposon End Prediction

Transposon ends were estimated from intergenic regions flanking the effector and the transposon machinery. For example, for Cas12k CAST, the intergenic region located directly upstream from TnsB and directly downstream from the CRISPR locus, were predicted as containing the Tn7 transposon left and right ends (LE and RE) (see e.g., FIG. 11A, which shows LE and RE analysis in the context of an MG64-3 family CAST locus diagram).

Direct and inverted repeats (DR/IR) of ˜12 bp were predicted on the contig, with up to 2 mismatches. In addition, the Dotplot algorithm was used to find short (˜ 10-20 bp) DR/IR flanking CAST transposons. Matching DR/IR located in intergenic regions flanking CAST effector and transposon genes were predicted to encode transposon binding sites. LE and RE extracted from intergenic regions, which encode putative transposon binding sites, were aligned to define the transposon ends boundaries. Putative transposon LE and RE ends are identified as regions: a) located within 400 bp upstream and downstream from the first and last predicted transposon encoded genes; b) sharing multiple short inverted repeats; and c) sharing >65% nucleotide id. This process was repeated to identify putative LE/RE sequences for MG36-5 (SEQ ID NOs: 17-18), MG39-1 (SEQ ID NOs: 20-21), MG64-2 (SEQ ID NOs: 125-126), MG64-4 (SEQ ID NOs: 127-128), MG64-6 (SEQ ID NOs: 123-124), MG64-7 (SEQ ID NOs: 129-130), MG64-13 (SEQ ID NOs: 131-132), MG64-54 (SEQ ID NO: 133), MG108-1 (SEQ ID NOs: 134-135), MG110-1 (SEQ ID NOs: 136-137), and MG110-2 (SEQ ID NOs: 138-139).

### Example 15—Single Guide Design for Class 2, Type V CAST Systems

Analysis of the intergenic regions surrounding the Cas effector and CRISPR array for MG 64 sub-families identified a potential anti-repeat sequence and a conserved “CYCC (N6) GGRG” stem-loop structure neighboring the antirepeat corresponding to the sequence of the tracrRNA (FIG. 11B). TracrRNA and crRNA repeat were folded and trimmed, adding a tetraloop sequence of GAAA to maintain the stem loop region of the crRNA-tracrRNA complementary sequence, in order to generate the sgRNA. These sequences are outlined in Table 2 below.

TABLE 2
Corresponding crRNA-tracrRNA sequences for MG families described herein.

| Description | SEQ ID NO: | Sequence |
|---|---|---|
| MG64-2 crRNA | 255 | See sequence listing |
| MG64-2 tracrRNA | 262 | AAUUAAUAGCGCCGCCGUUCAU |
| | | GCUUCUAGGAGCCUCUGAAAGG |
| | | UGACAAAUGCGGGUUAGUUUGG |
| | | CUGUUGUCAGACAGUCUUGCUU |
| | | UCUGACCCUGGUAGCUGCCCAC |
| | | CCCGAAGCUGCUGUUCCUUGUG |
| | | AACAGGAAUUAGGUGCGCCCCC |
| | | AGUAAUAAGGGUAUGGGUUUAC |
| | | CACAGUGGUGGCUACUGAAUCA |
| | | CCUCCGAGCAAGGAGGAACCCA |
| | | CU |
| MG64-4 crRNA | 256 | See sequence listing |
| MG64-4 tracrRNA | 209 | See sequence listing |
| MG64-6 crRNA | 257 | See sequence listing |
| MG64-6 tracrRNA | 263 | AUAACAGCGCCGCAGGUCAUGC |
| | | CGUCAAAAGCCUCUGAACUGUG |
| | | UUAAAUGGGGGUUAGUUUGACU |
| | | GUUGAAAGACAGUUGUGCUUUC |
| | | UGACCCUGGUAGCUGCCCACCC |
| | | UGAUGCUGCUAUCUUUCGGGAU |
| | | AGGAAUAAGGUGCGCUCCCAGU |
| | | AAUAGGGGUGUAGAUGUACUAC |
| | | AGUGGUGGCUACUAAAUCACCU |
| | | CCGACCAAGGAGGAAUCCAUCC |
| | | UUAAUUUUUUAUUUUUU |
| MG64-7 crRNA | 258 | See sequence listing |
| MG64-7 tracrRNA | 210 | See sequence listing |
| MG108-1 crRNA | 261 | See sequence listing |
| MG108-1 tracrRNA | 235 | See sequence listing |
| MG108-2 crRNA | 260 | See sequence listing |
| MG108-2 tracrRNA | 236 | See sequence listing |

### Example 16—In Vitro Integration Activity Using Targeted Nuclease

In situ expression and protein sequence analyses indicated that some RNA guided effectors are active nucleases. They contain predicted endonuclease-associated domains (matching RuvC and HNH_endonuclease domains), and/or predicted HNH and RuvC catalytic residues. Candidate activity was tested with engineered single guide RNA sequences using the in vitro expression system and in vitro transcribed RNA. Active proteins are identified as those that successfully cleave the library to yield a band around 170 bp in agarose gel electrophoresis.

### Example 17—Programmable DNA Integration

CAST activity was tested by combining five types of components in a single reaction: (1) a Cas effector protein expressed by an in vitro expression system; (2) a target DNA fragment or plasmid containing the target sequence and PAM corresponding to the Cas enzyme; (3) a donor DNA fragment containing a marker or fragment of DNA flanked by the predicted LE and RE of the transposase system in a DNA fragment or plasmid; (4) any combination of additional transposase proteins predicted to be part of the array expressed using an in vitro expression system; and (5) an engineered in vitro transcribed single guide RNA sequence. Active systems that successfully transposed the donor fragment were assayed by PCR amplification of the donor-target junction.

FIG. 13 shows example data demonstrating that the MG64-6 system comprising the MG64-6 effector, TnsB, TnsC, and TniQ proteins (SEQ ID NOs: 30-33) using the predicted LE/RE donor sequences (SEQ ID NOs: 123-124) and in silico designed sgRNA (SEQ ID NO:201) is active. After performing the transposition reaction by combining all the MG64-6 components, PCR amplification of the junction showed that proper donor-target formation occurred and the transposition reaction was sg dependent. (FIG. 13A). Presence of amplified bands in PCR reactions #3 and #4 (spanning the LE/RE junctions when the LE/RE is inserted distal to the PAM, respectively) indicated that both orientations of the donor relative to the target are made: one where the LE is closer to the PAM, and one where the RE is closer to the PAM. While both transposition orientations were made, there was a preference for donor integration in the target where the LE is closer to the PAM, represented by strong band present for reactions #4 and #5 (which span the LE junction when it is inserted distal to the PAM and the RE junction when it is inserted proximal to the PAM, respectively).

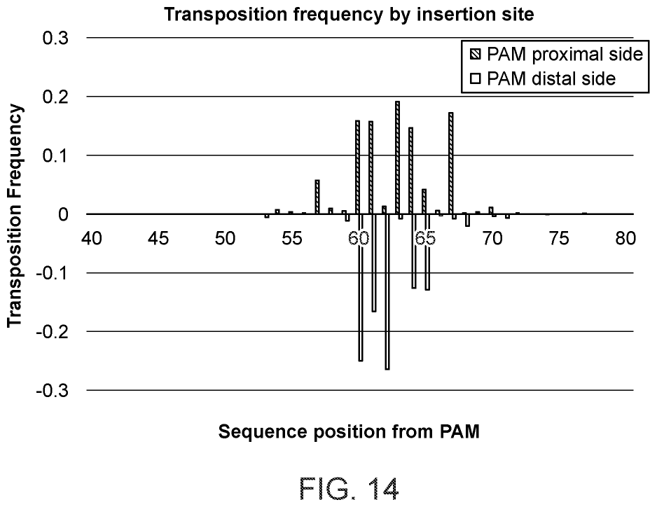

Sanger sequencing of the preferred orientation product was performed. Of the integrations that occur with the LE closer to the PAM, there was a clear degradation of the sequencing chromatogram signal from either the forward or reverse direction over the target/donor junction (FIG. 13C). This indicated that, of the products that are oriented with the LE closer to the PAM, integration occurred over a range of nucleotides, with the primary product of LE-closer-to-PAM products as a 61 bp integration from the PAM (FIG. 14). Sequencing that originated from the donor over the donor-target junction defined the composition of the essential outer bounds of the LE and RE sequences. Further investigation of the LE and RE domains will determine the inner limits of the LE and RE sequences and thus the minimal LE/RE that are essential for transposition. Sequencing of the RE on LE-closer-to-PAM products showed a 3 bp duplication downstream of the donor RE. This is in part due to the Tn7 transposase integration event that cleaves and ligates the donor fragment at a staggered cut site. A 3 bp duplication is smaller than the expected 5 bp of duplication from other Tn7 transposases.

Sanger sequencing of the PCR amplified product over the 8N library of the target plasmid also elucidated that the PAM preference of the MG64-6 effector as a nGTn/nGTt on the 5′ end of the spacer. NGS analysis of the PAM library target corroborated the nGTn motif selectivity at the 5′ end (FIG. 13B).

### Example 18—Integration Window Determination

PCR junctions of the PAM that were amplified in Example 17 above were indexed for NGS libraries and sequenced. Reads were mapped and quantified using CRISPResso using an amplicon sequence of a putative transposition sequence with a 60 bp distance of integration from the PAM (guideseq=20 bp 3′ end of LE or RE, center of window=0, window size=20) Indel histogram was normalized to total indel reads detected, and frequencies were plotted relative to the 60 bp reference sequence (FIG. 14).

Both PCR reactions 5 (LE proximal to PAM, FIG. 13A) and PCR 4 (RE distal to PAM, FIG. 13B) were plotted on the sequence and distance from the PAM for MG64-6 (FIG. 14). Analysis of the integration window indicated that 95% of the integrations that occurred at the spacer PAM site are within a 10 bp window between 58 and 68 nucleotides away from the PAM. Differences in the integration distance between the distal and the proximal frequencies reflected the integration site duplication—a 3-5 base pair duplication as a result of staggered nuclease activity of the transposase upon integration.

### Example 19—Transposon End Verification Via Gel Shift

In order to verify the activity of TnsB on the predicted transposon end sequence, the RE of MG64-6 was amplified using FAM labeled oligos. MG64-6 TnsB protein was expressed using a cell free transcription/translation system and incubated with the RE FAM labeled product. After incubation for 30 minutes, binding was observed on a native 5% TBE gel (FIG. 15). Multiple bands of fluorescent product within the co-incubated lane (FIG. 15, lane 3) indicated a minimum of 3 TnsB binding sites.

### Example 20—Colony PCR Screen of Transposase Activity (Prophetic)

Transposition activity is assayed via a colony PCR screen. After transformation with the pDonor plasmids, E. coli are plated onto LB-agar containing ampicillin, chloramphenicol, and tetracycline. Select CFUs are added to a solution containing PCR reagents and primers that flank the insertion junction.

### Example 21—LE-RE Minimization (Prophetic)

Sequencing of the target-transposition junction helps to identify the terminal inverted repeats by identifying the outmost sequence from the donor plasmid that are incorporated into the target reaction. By performing repeat analysis of 14 bp with variability of 10%, short repeats contained within the terminal ends are identified; identifying the minimal sequences to be included in truncations of these that preserve the repeats while deleting superfluous sequence. Prediction and cloning is done in multiple iterations, with each interaction tested with in vitro transposition. Transposition is predicted to be active down to a LE region of 68 bp combined with a RE region of 96 bp.

### Example 22—Overhang Influence of Transposition (Prophetic)

In order to test whether superfluous sequence outside of the TnsB binding motifs are necessary for transposition, oligos designed for the TGTACA or TGTCGA motifs of both LE and RE are designed and synthesized with 0, 1, 2, 3, 5 and 10 bp extra base pairs. These synthesized oligos are used to generate donor PCR fragments with overhangs and tested for their ability to transpose into the target site.

### Example 23—CAST NLS Design (Prophetic)

Eukaryotic genome editing for therapeutic purposes is dependent on the import of editing enzymes into the nucleus. Small polypeptide stretches of larger proteins signal to cellular components for protein import across the nuclear membrane. Placement of these tags may require optimization, as import function versus function of the protein to which it is fused are potential tradeoffs depending on the location of the NLS tag. In order to test functional orientations of the NLS to each of the components of the CAST complex, constructs fusing Nucleoplasmin NLS to the N-terminus and SV40 NLS to the C-terminus of each of the components of the MG CAST are synthesized. Proteins of these constructs are expressed in cell free in vitro transcription/translation reactions and tested for in vitro transposition activity with a complement set of untagged components. NLS-tagged constructs are assessed for maintenance of activity by PCR of the donor-target junction using PCR 4 (Assessing RE distal transpositions) and the cognate transposition event, PCR 5 (Assessing LE proximal transposition).

### Example 24—Cas12k and TniQ Protein Fusion Construct Design and Testing (Prophetic)

To simplify/minimize the expression of the protein components and facilitate delivery of these components into cells, fusion constructs between the Cas12k effector and the TniQ protein with various linkers, linker lengths, and domain boundaries are designed, synthesized, and tested. Both orientations of the TniQ fused to the Cas12k are designed and synthesized, a C-terminal fusion, Cas-TniQ, and an N-terminal fusion, TniQ-Cas.

Two other linkers are also employed to fuse the effector and TniQ genes. P2A, a self-stopping translation sequence is active in a Cas-NLS-P2A-NLS-TniQ construct, and an MCV Internal Ribosome Entry Sequence (IRES) mRNA-based linker allows for independent translation of the two components in cells.

### Example 25—Intracellular Expression Coupled with In Vitro Transposition Testing (Prophetic)

To test the functionality of the NLS constructs in a physiologically relevant environment, constructs cloned with active NLS-tagged CAST components are integrated into K562 cells using lentiviral transduction. Briefly, constructs cloned into lentiviral transfer plasmids are transfected into 293T cells with envelope and packaging plasmids, and virus containing supernatant are harvested from the media after 72 hr incubation. Media containing virus is then incubated with K562 cell lines with 8 μg/mL of polybrene for 72 hrs, and transfected cells are then selected for integration in bulk using Puromycin at 1 μg/mL for 4 days. Cell lines undergoing selection are harvested at the end of 4 days, and differentially lysed for nuclear and cytoplasmic fractions. Subsequent fractions are then tested for transposition capability with a complementary set of in vitro expressed components.

Both NLS-TnsB and TnsB-NLS are tested by cell fractionation and in vitro transposition, and transposition is detected across both cytoplasmic and nuclear fractions

Cas12k fusions in the cell are similarly fractionated and tested for transposition. Cas-NLS Cas-NLS-P2A-NLS-TniQ are transduced into cells, fractionated, and tested in vitro for subcellular activity. Cas-NLS-P2A-NLS-TniQ is able to transpose in the cytoplasm with the addition of single guide to the reaction. By supplementing holo Cas protein (+sgRNA) or additional TniQ with sgRNA, the Cas-NLS-P2A-NLS-TniQ construct can be complemented in the nuclear fraction.

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Example 26-Class 2 Cas12k CAST System Prediction

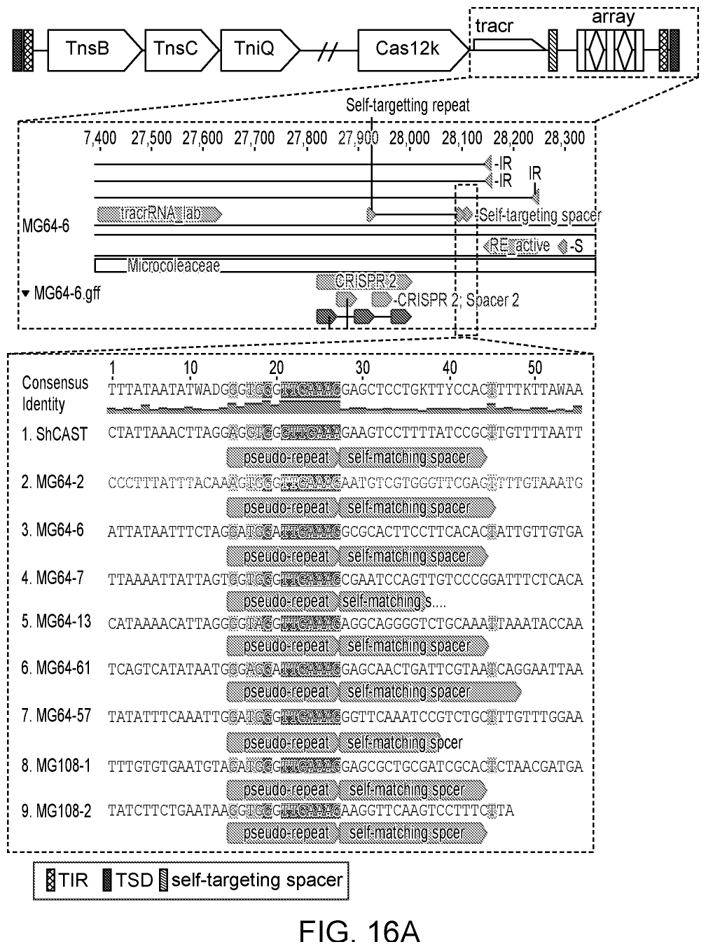

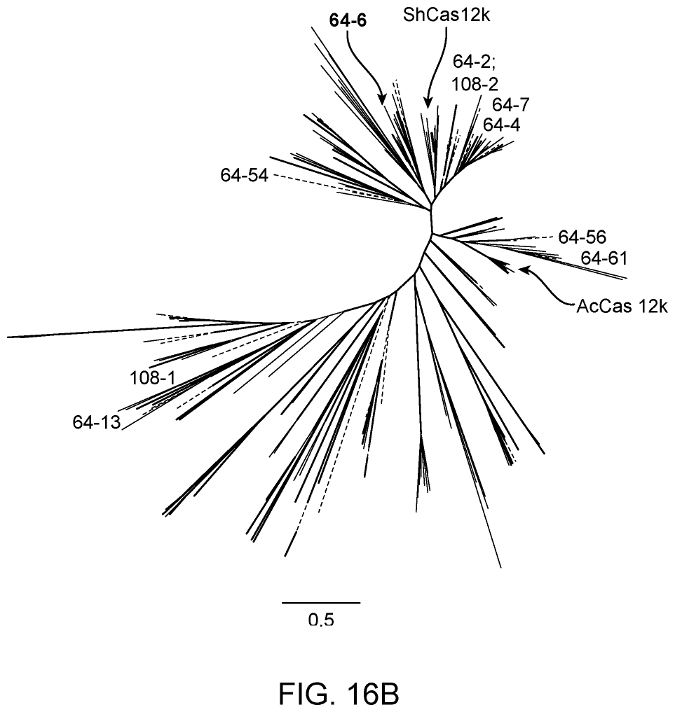

Cas12k CAST systems encode a nuclease-defective CRISPR Cas12k effector, a CRISPR array, a tracrRNA, and Tn5053-like transposition proteins (FIG. 16A). Cas12k effectors are phylogenetically diverse and features that establish their association with CASTs have been confirmed (FIGS. 16A-16B). For example, the transposon left end was identified downstream from many Cas12k effectors and their CRISPR locus, as shown by terminal inverted repeats and self-matching spacer sequences (FIG. 16A (center) and FIG. 16B).

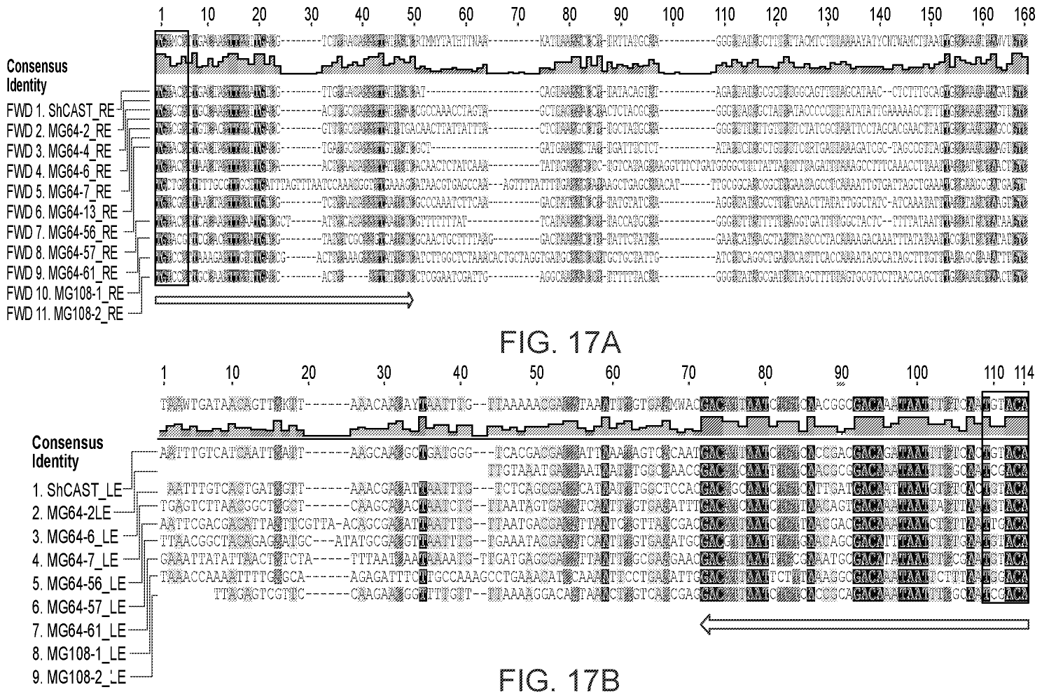

Transposon ends of Cas12k CAST systems were determined from intergenic regions flanking the CRISPR locus and the transposon machinery. For example, the intergenic region located directly upstream from TnsB and directly downstream from the CRISPR locus, were predicted as containing the transposon left and right ends (LE and RE). These intergenic regions were aligned among several homologs and regions of conservation were used to predict the transposon ends boundaries (FIG. 17).

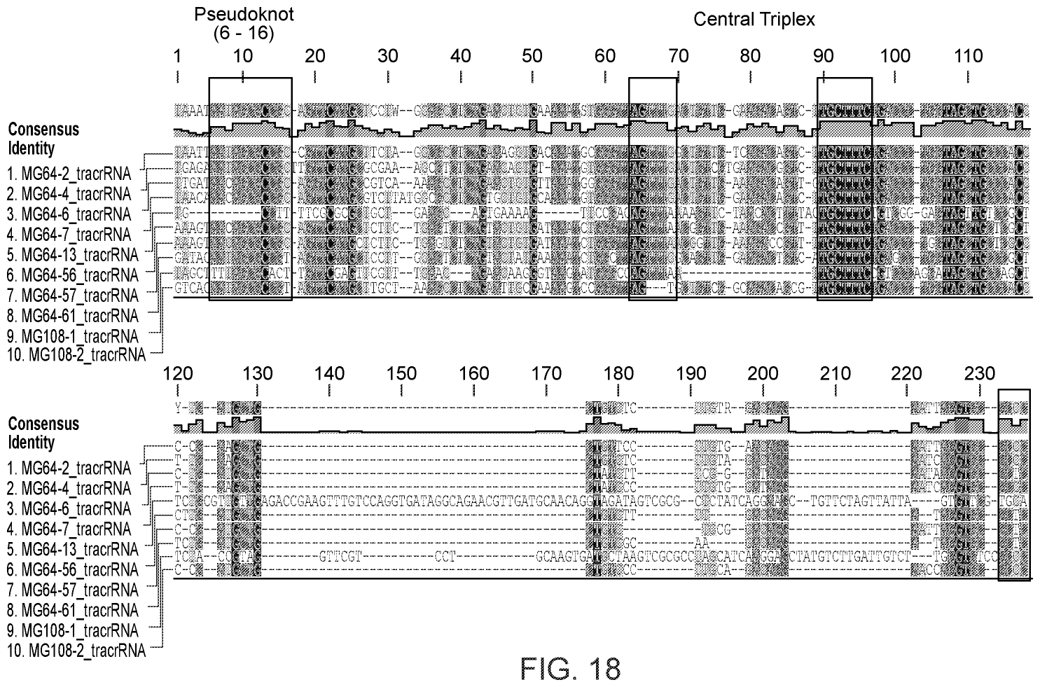

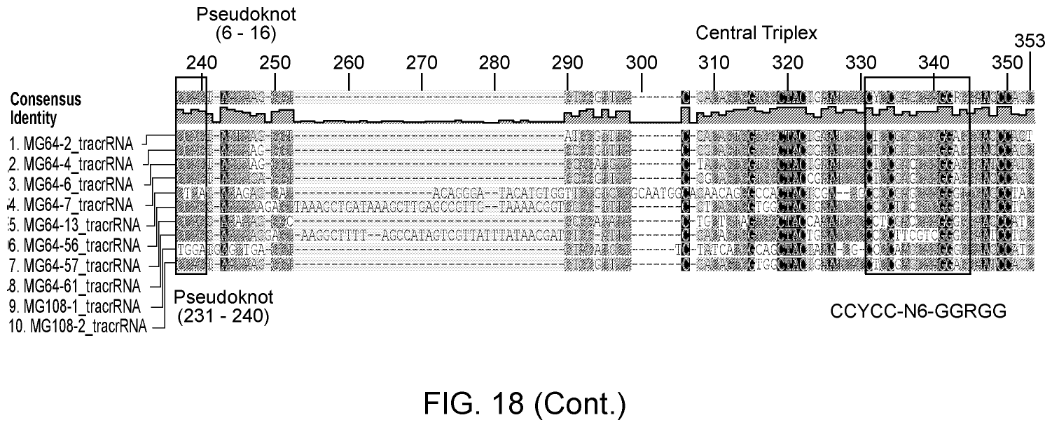

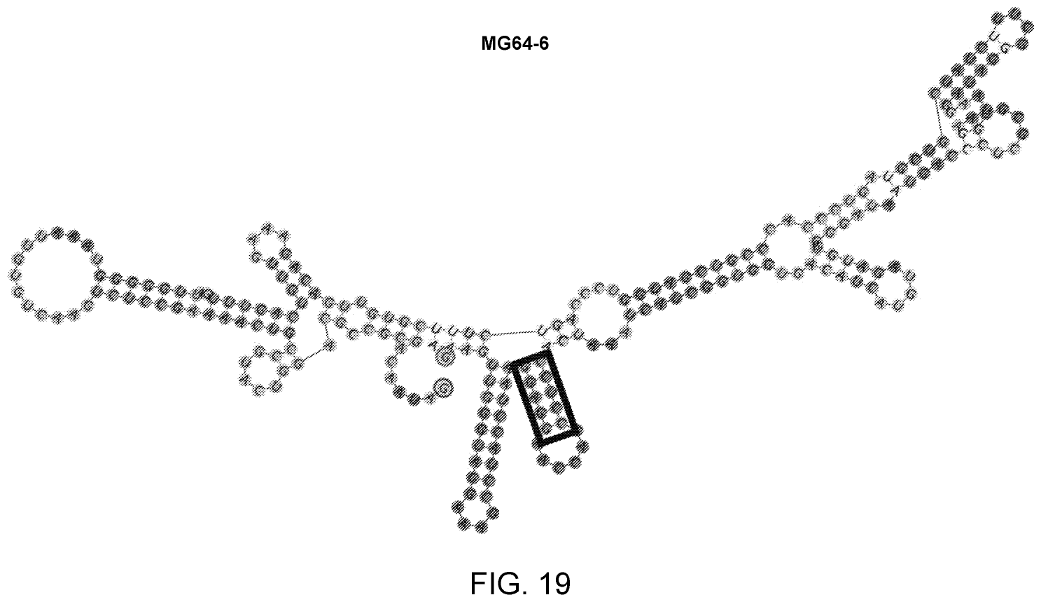

The 3′ end of Cas12k CAST CRISPR repeats (crRNA) contain a conserved motif 5′-GNNGGNNTGAAAG-3′ when aligned among homologs, and they are predicted to bind to different regions of the tracrRNA to form secondary and tertiary guide RNA structures (FIG. 18 and FIG. 19). Self-matching spacers within the CAST transposon are often found next to a pseudo CRISPR repeat in the vicinity of the CRISPR arrays (FIG. 16A, bottom alignment).

Analysis of the intergenic regions surrounding the Cas effector and CRISPR array identified a potential anti-repeat sequence and a conserved “CCYCC (n6) GGRGG” stem-loop structure neighboring the antirepeat, corresponding to the duplexing sequence of the tracrRNA (FIG. 18). Good quality tracrRNAs were used to build covariance models and searched on all Cas12k CAST genomic fragments identified in this study.

For single guide RNA (sgRNA) design, tracrRNA and crRNA repeat were folded and trimmed, adding a tetraloop sequence of GAAA to maintain the stem loop region of the crRNA-tracrRNA complementary sequence (FIG. 19). Generally, sgRNAs share conserved structural features despite sharing less than 70% pairwise nucleotide identity (FIG. 18).

### Example 27-In Vitro Characterization of Novel Cas12k CAST Systems

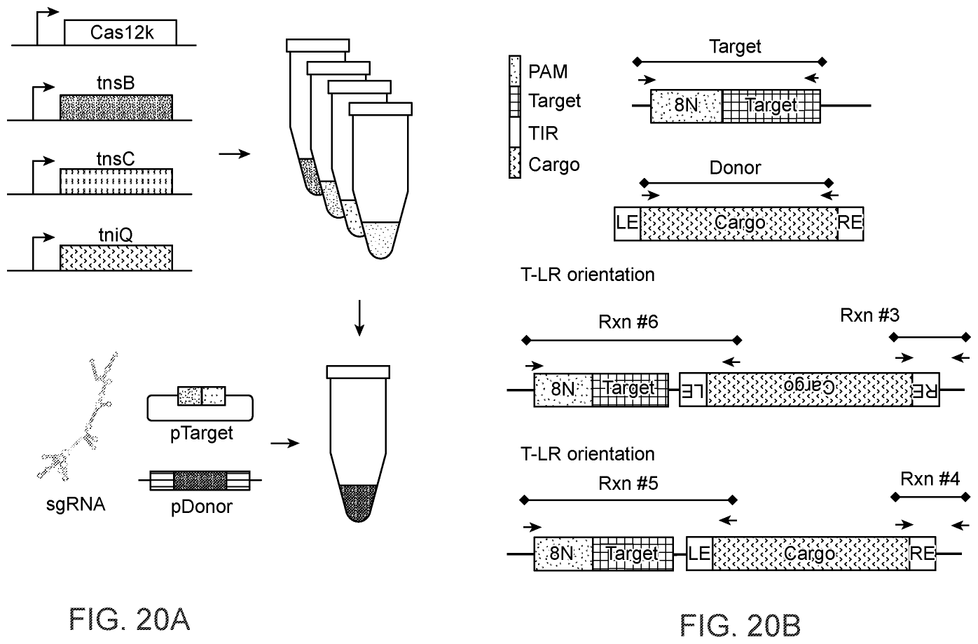

In order to test the function of Cas12k CAST systems and elucidate the potential PAM, a transposition reaction was assembled using synthesized Cas12k effectors and Tn5053-like proteins under the control of a T7 promoter. Each open reading frame was expressed in vitro with an in vitro expression system and assembled in a transposition reaction with a transposition buffer, a donor PCR fragment, and a plasmid based target with an 8N target library (FIG. 20A). When CAST systems are active and can transpose the donor fragment into the library of target plasmids, the transposition reaction can be PCR amplified to recover each donor-target junction of the two potential products of transposition (FIG. 20B).

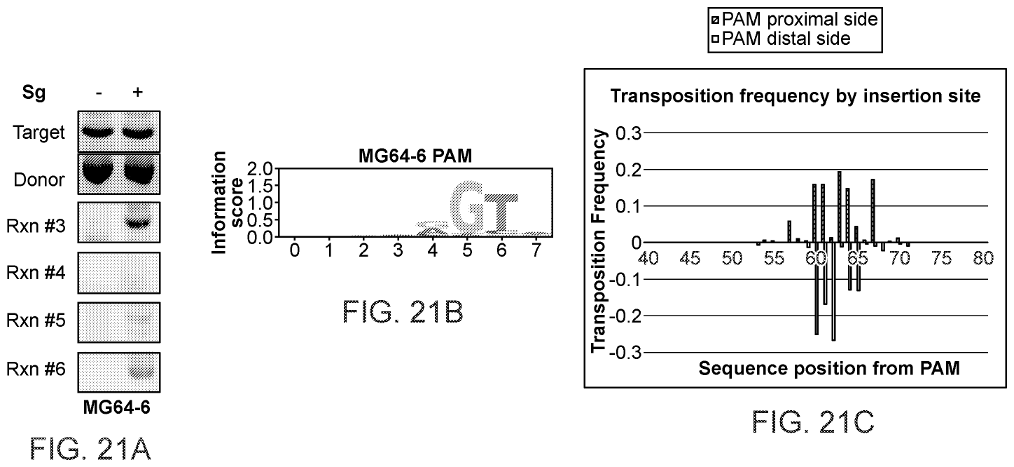

Of the discovered Cas12k CAST systems, ten systems were prioritized for their novelty and completeness and tested for transposition potential in vitro. MG64-6 CAST was able to transpose the cargo to the donor plasmid in an sgRNA-dependent manner (FIG. 21A). For each of the potential junction PCR amplifications, it was predicted that all four junctions would be observed if both orientations of integration were complete, or only two of the junctions if integration only occurred in a single orientation (FIG. 20B). Surprisingly, robust transposition was observed in three of the four junction PCR reactions. The observed reactions represent both potential Left End junction products, in both Target-LE-Cargo-RE (T-LR) and Target-RE-Cargo-LE (T-RL) orientations (PCR5 and PCR3 respectively), and the T-LR oriented right end product (PCR4) (FIG. 21A)

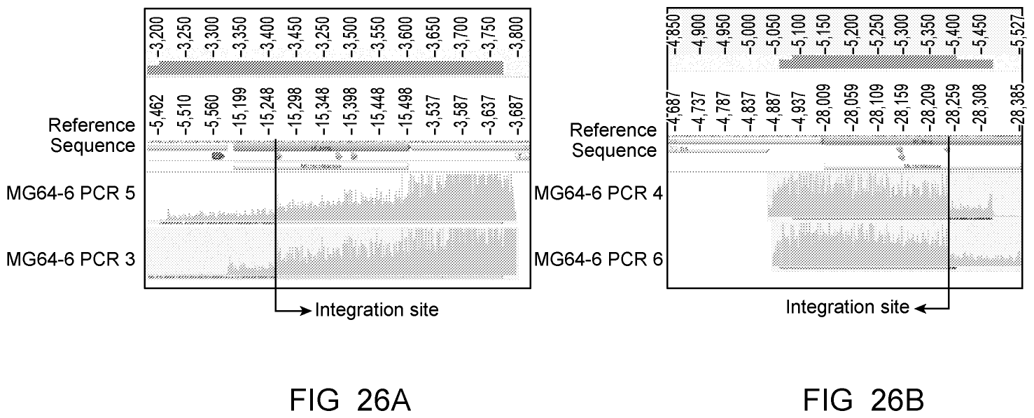

Sanger-based sequencing of the PCR transposition fragments accurately aligned to the target sequence and the sequencing signal quickly degraded at the target-donor junction (FIG. 26). Signal degradation indicates a population of integration products and sequencing from the donor end of the transposition products verified both the LE and RE prediction for the MG64-6 system.

To clucidate the PAM preference of the active CAST systems, successful transposition events from the population of the 8N randomized library that received the donor sequence were sequenced via NGS. Sequencing reads identified an rGTN-5′ PAM for MG64-6 (FIG. 21B).

In addition to identifying the PAM, it is also beneficial to determine the integration window size: the respective distance from the PAM where integration occurs. Cas12k CASTs integrate cargo at a specific window 60 bp offset from the PAM motif. When NGS reads of the transposition junction for MG64-6 were quantified, it was determined that 99% of the integration events occurred between 57 and 67 base pairs away from the PAM (FIG. 21C).

### Example 28—E. coli Integration Activity with MG64-6

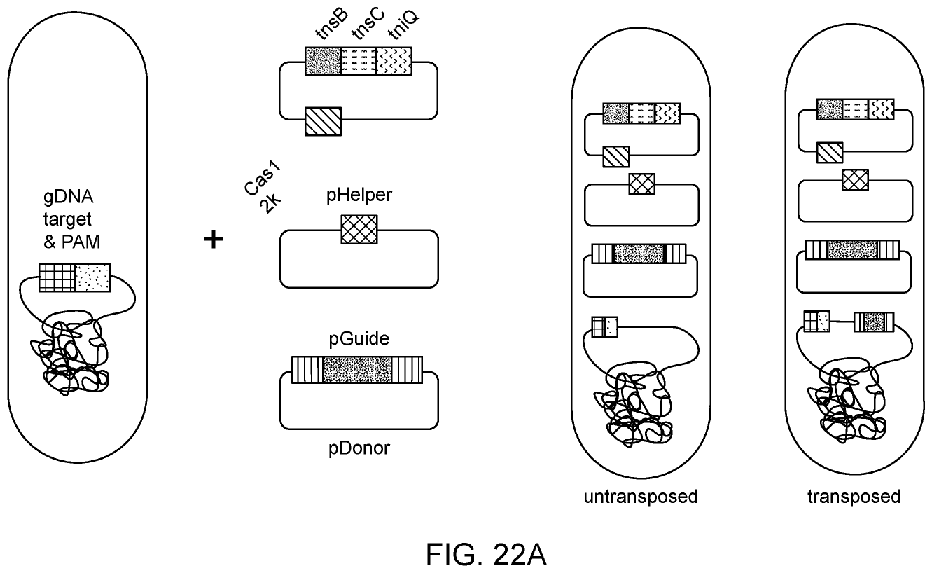

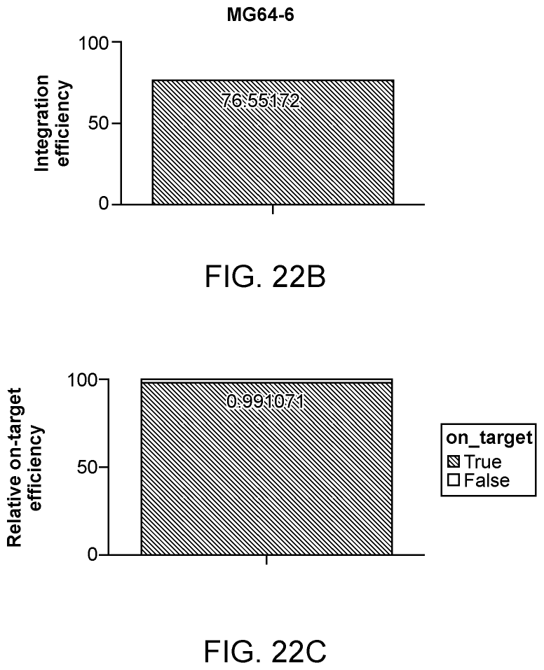

To test the transposition efficiency in a cellular context, a strain of E. coli BL21 (DE3) was engineered to include the spacer sequence confirmed for activity in vitro. A plasmid containing the polycistronic Tn5053-like genes and the effector under the T7 promoter was used to express the CAST proteins, and a separate plasmid was co-transformed to introduce the guide under the control of the J23119 promoter (FIG. 22A). The pDonor plasmids contained an antibiotic resistance cargo flanked by the confirmed WT LE and RE and the minimized LE and RE for MG64-6.

An NGS based method was developed to assess transposition efficiency for MG64-6. NGS reads indicate over 75% editing efficiency (FIG. 22B) and enabled determination of the off-target profile associated with MG64-6. The off-target editing rate was determined as a single NGS read that mapped to the LE or RE with an additional 14 bases mapping elsewhere in the E. coli genome. Off-target integration greater than 1% of all the summed transposition events was not detected (FIG. 22C).

### Example 29-Endogenous Locus Targeting

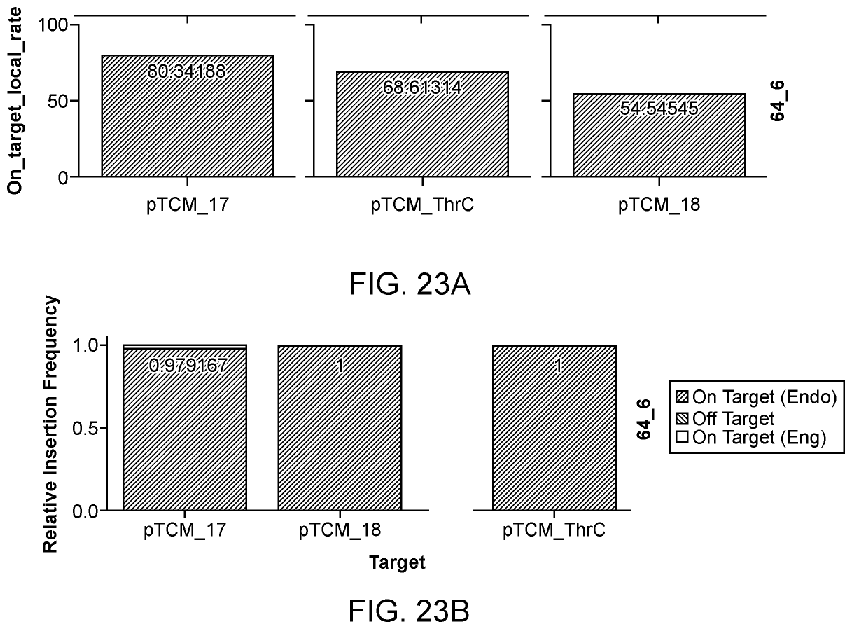

In order to test the programmability of these systems to integrate into the E. coli genome, three target sites with rGTN-5′ PAMs were chosen to integrate into. From WGS data, MG64-6 CAST was able to integrate at multiple loci with efficiencies ranging between 54-80% (FIG. 23A). Together with the low off-target rate, these data demonstrate that this Cas12k system is capable of achieving high rates of genomic integration with a programmable RNA guide (FIG. 23B).

### Example 30—CAST NLS Design

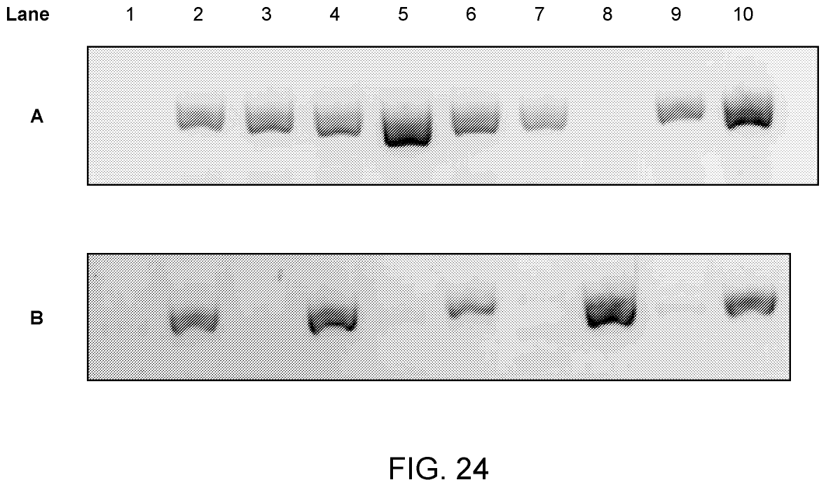

Eukaryotic genome editing for therapeutic purposes is largely dependent on the import of editing enzymes into the nucleus. Small polypeptide stretches of larger proteins signal to cellular components for protein import across the nuclear membrane. Placement of these tags is not trivial, as these NLS tags need to provide import function while also maintaining function of the protein to which it is fused. In order to test functional orientations of the NLS to each of the components of the MG64-6 CAST complex, constructs fusing Nucleoplasmin NLS to the N-terminus and SV40 NLS to the C-terminus of each of the components of the CAST were designed and synthesized. Proteins of these constructs were expressed in cell-free in vitro transcription/translation reactions and tested for in vitro transposition activity with a complement set of untagged components. NLS-tagged constructs were assessed for maintenance of activity by PCR of the donor-target junction using PCR 5 (LE to proximal transposition) (Panel A of FIG. 24).

### Example 31—Combinatorial NLS Testing of NLS CAST Components

In order to test the abilities of the NLS components to properly interact with each component of the complex, both N terminal and C terminal tagged NLS constructs of each tnsB, tniQ, and Cas12k with NLS-tnsC were tested combinatorically. From the ability to transpose, it was observed that tniQ-NLS was the more robust orientation for NLS fused to tniQ and the strongest transposition with the inclusion of NLS-Cas12k in a transposition reaction (Panel B of FIG. 24, lane 8).

### Example 32—Intracellular Expression Coupled with In Vitro Transposition Testing

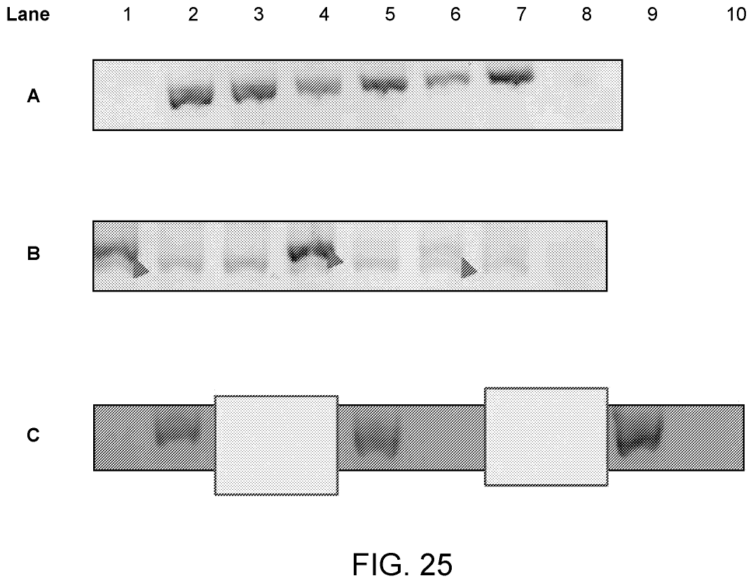

To test the functionality of the NLS constructs in a physiologically relevant environment, constructs cloned with active NLS-tagged CAST components were integrated into K562 cells using lentiviral transduction. All NLS components that were active in vitro were also active in cytoplasmic fractions except for NLS-TniQ (Panels A and B of FIG. 25). Nuclear fractions tested indicated a strong transposition in NLS-TnsB (Panel B of FIG. 25, lane 4), TnsB-NLS (Panel B of FIG. 25, lane 5), and NLS-TnsC (Panel B of FIG. 25, lane 6).

In order to test the ability of multiple constructs to be expressed and imported into the nucleus, both NLS-TnsB and TnsB-NLS co-expressed with NLS-TnsC were tested by cell fractionation and in vitro transposition. From the cell fractionation experiments and testing in vitro, co-expressed NLS-TnsB with NLS-TnsC was active in both cytoplasmic and nuclear fractions (Panel C of FIG. 25, lanes 5 & 9).

### Example 33—In Vitro Targeted Integrase Activity of MG64-6 CAST with Homologous CAST Components

### In Vitro Targeted Integrase Activity Experiments

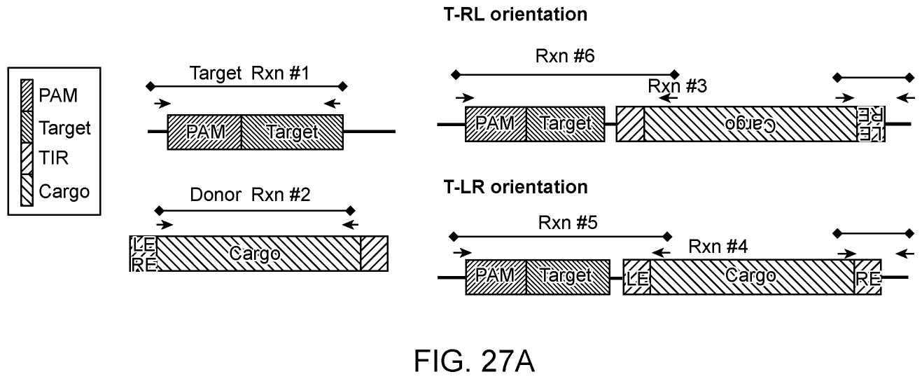

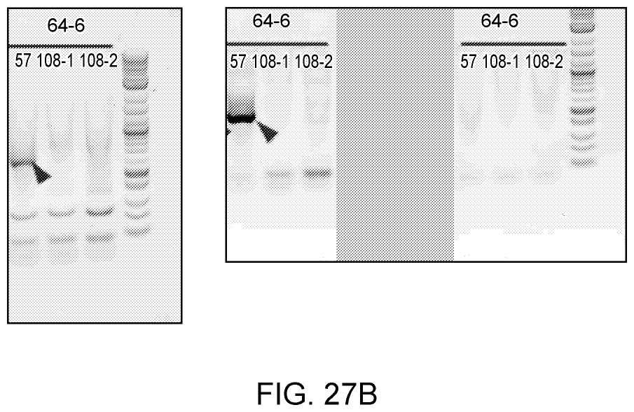

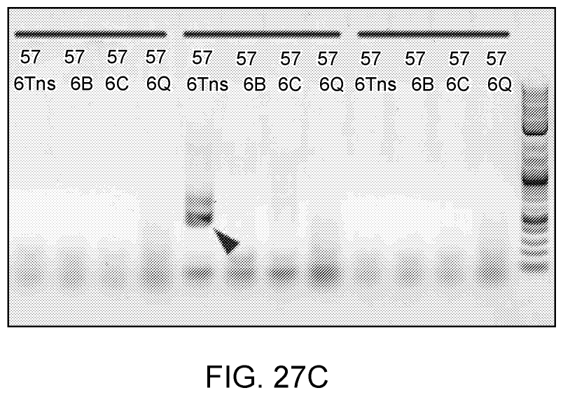

Integrase activity was assayed with a substrate containing the required 5′ NGT PAM adjacent to the spacer sequence (FIG. 27A). T7 promoter sequences were introduced by PCR amplification of all transposase, single guide and effector components, and expressed independently in an in vitro transcription/translation system. Purified in vitro transcribed single guide RNA for MG64-6, as well as those associated with homologous Cas12k effectors was refolded in duplex buffer (10 mM Tris pH 7.0, 150 mM NaCl, 1 mM MgCl2) and normalized to 1 μM. Donor fragments were PCR amplified from plasmid pDonors of the respective systems, which contain a kanamycin or tetracycline resistance marker flanked by left end (LE) and right end (RE) transposon motifs for integration and normalized to 50 ng/μL.

After expression, 1 μL of Cas12k in vitro expression reaction was added to 0.5 picomoles of sgRNA and incubated for 20 minutes at 25° C. Individually expressed transposase proteins were then added volumetrically at 1 μL per expression. 50 ng of target DNA and 50 ng donor DNA were then added to the transposition reaction in a reaction buffer, with final concentrations of 26 mM HEPES pH 7.5, 4.2 mM TRIS pH 8, 50 μg/mL BSA, 2 mM ATP, 2.1 mM TCEP, 0.05 mM EDTA, 0.2 mM MgCl2, 28 mM NaCl, 21 mM KCl, 1.35% glycerol (final pH 7.5), and 15 mM Mg(OAc)₂. In vitro transposition reactions were performed at 37° C. for 2 hours, transposition reactions were diluted tenfold in water, and used subsequently as a template for junction PCR analysis.

### Junction PCT Analysis

Junction PCR reactions were performed with Q5 polymerase and amplified with primers flanking: Rxn #1 (Target), Rxn #2 (Donor), Rxn #5 (Forward LE), Rxn #4 (Forward RE), Rxn #3 (Reverse LE), and Rxn #6 (Reverse RE) (FIG. 27A). PCR fragments were run on a 2% agarose gel in 1×TAE and analyzed for size discrimination. Appropriately sized bands of each PCR junction were then gel excised, and the PCR fragments were recovered through purification and sanger sequenced using both amplification primers. Resulting Sanger sequencing was mapped to a putative forward or reverse integration at ˜60 bp away from the PAM.

### Results: Single Guide RNA and Cas12k Effector Swapping Experiments

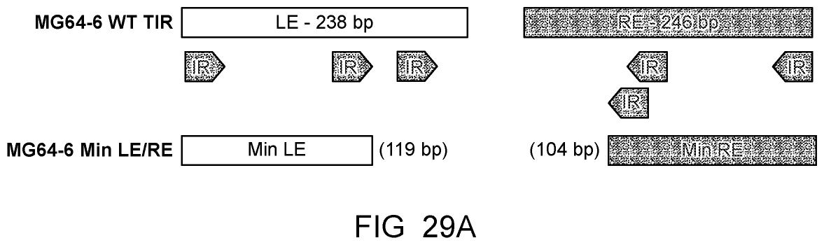

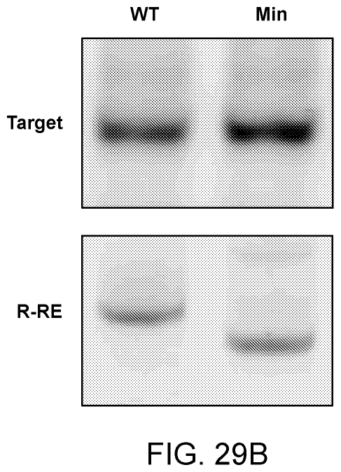

We tested the cross-functional potential of predicted single guide RNAs (sgRNAs) associated with diverse Cas12k effectors that could complement the MG64-6 CAST system (SEQ ID No. 30-33). sgRNA of effector MG64-57 (SEQ ID No. 491) is recognized by the MG64-6 CAST system for targetable cargo integration, as demonstrated by the expected PCR product bands of integration junctions reactions (FIG. 29B). In addition, the ability of the MG64-6 CAST system to promote targetable integration with Cas12k effectors from homologous systems was evaluated. The wild type MG64-6 Cas12k (SEQ ID No. 30) was replaced for effectors from alternative CAST systems, the MG64-6 transposon suite (SEQ ID No. 31-33) was functional with the Cas12k effector from MG64-57 (SEQ ID No. 264), as demonstrated by the expected PCR product bands of integration junctions reactions Rxn #3 or Rxn #5 (FIG. 27C).

### Example 34—NLS-CAST Component Immunofluorescence

### Fixed Cell Staining Shows NLS-CAST Components are all Capable of Translocation to the Nucleus

In order to test functional orientations of NLS tags to each of the components of the CAST complex for Eukaryotic nuclear import, constructs were designed and synthesized fusing Nucleoplasmin NLS to the N-terminus and SV40 NLS to the C-terminus of each of the components of the MG CAST. Proteins were expressed in cell free in vitro transcription/translation reactions. By exposing the CAST-NLS proteins in cells whether through genomic expression or in the form of mRNA, the cells are fixed and a stain is performed for the introduced epitope tag using fluorescently conjugated antibodies. By visualizing the fluorescence relative to DAPI nuclear staining, the likelihood of the protein to be translocated into the nucleus for activity can be determined.

### Immunofluorescence in HEK293T Cells

HEK293T cells were transduced with Lentivirus containing transfer plasmid containing MG64-6 NLS-HA-Cas12k, NLS-HA-TnsB, NLS-FLAG-TnsC, or TniQ-FLAG-NLS co-expressed with Puromycin. NLS component expressing cells were selected using Puromycin at 2 μg/mL for 96 hours refreshing Puromycin every 48 hrs. Selected protein NLS-CAST expressing cells were plated on a collagen coated coverslip at 50,000 cells per 24-well plate. Cell cultures were left to adhere to the cover slip overnight. After 48 hours of expression, cells were fixed using 4% formaldehyde, cell membranes were permeabilized with Triton X-100, then washed with 2% BSA and probed overnight with anti-HA antibody (NLS-HA-Cas12k and NLS-HA-TnsB) or anti-FLAG antibody (NLS-FLAG-TnsC and TniQ-FLAG-TniQ). Cells were then washed with 2% BSA in PBS and then subsequently stained with FITC conjugated goat anti-Mouse secondary antibody. Post secondary antibody exposure, cells were washed with PBS, and mounted on DAPI mounting epoxy and cured overnight. Visualization of cells was performed on an imaging system for fluorescence and nuclear localization was determined by FITC co-localization with DAPI staining.

Results: All NLS-CAST Components Co-Localize with DAPI in the Nucleoplasm

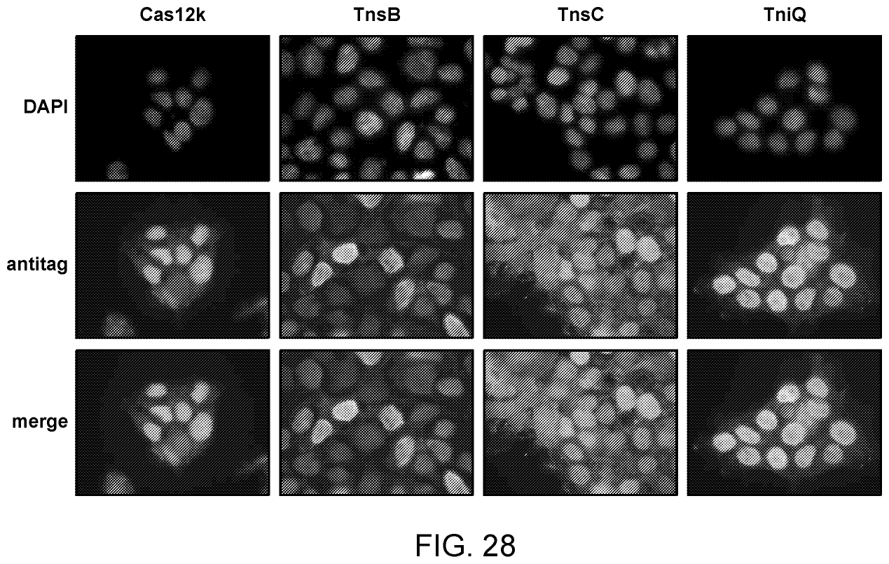

DAPI stains DNA and is the reference stain for nuclear localization in fixed cells. All cells were able to localize DAPI into the nucleus indicating sufficient fixing and permeabilization (FIG. 28, row 1). NLS-HA-Cas12k, NLS-HA-TnsB, and TniQ-FLAG-NLS TnsC-FLAG-NLS of the MG64-6 CAST complex were capable of localizing to the nucleoplasm. However, TnsC was incomplete in its translocation into the nuclear compartment (FIG. 28, rows 2-3).

### Example 35—LE-RE Minimization

### Terminal Inverted Repeat Identification Enables Design of Minimized LE/RE for MG64-6

Sequencing of the target-transposition junction helped to identify the terminal inverted repeats by identifying the outmost sequence from the donor plasmid that was incorporated into the target reaction. By performing repeat analysis of 14 bp with variability of 10%, short repeats contained within the terminal ends were identified and truncations to preserve the TIR repeats while deleting unneeded sequences were designed.

### Results

LE was minimized by length through a series of truncations and internal deletions (SEQ ID No. 354-363): 133 bp (105 internal deletion) [LE2], 161 bp (77 bp internal deletion) [LE5], 119 bp [LE1], 146 bp [LE3], and 158 bp [LE4] truncations were tested. In addition, the RE was minimized by a series of truncations: 104 bp [RE1], 124 bp [RE2], 165 bp [RE4], and 186 bp (60 bp internal deletion) [RE5] were tested (FIG. 29A). In vitro transposition assays with MG64-6 CAST indicated that the minimized LE1 (119 bp) (SEQ ID No. 354) and RE1 (104 bp) (SEQ ID No. 359) were active (FIG. 29B). Together, this represents a minimization of TIR at >50% the WT size.

### Example 36—Ribosomal Protein S15 Homologs for Targeted Integration

### Results: Bioinformatic Discovery of RPS15

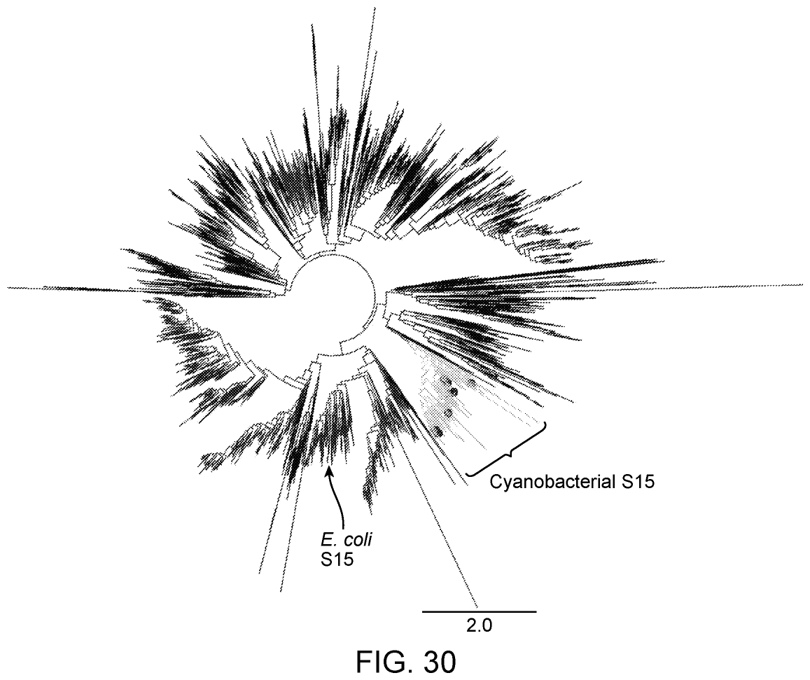

Recently, the small prokaryotic ribosomal protein subunit S15 was deemed necessary for targeted transposition by Cas12k CAST in vitro (Schmitz et al. (2022), Cell 185 (26); Park et al. (2022), Nature 613, 775-782). Ribosomal protein S15 distant homologs were identified from Pfam PF00312 domain searches with significant e-value of 1e⁻⁵. Of >1 million S15 protein hits, nearly 3,500 full-length, unique S15 sequences were identified in metagenomic assemblies in which Cas12k CAST effectors were also identified. Clustering at 99% average amino acid identity enabled classification of nearly 2,700 S15 cluster members by taxonomic affiliation, of which 166 (SEQ ID NOs. 494-659) were derived from Cyanobacteria (FIG. 30). Eight ribosomal protein S15 candidate sequences (MG190-8, MG190-33, MG190-35, MG190-43, MG190-84, MG190-109, MG190-171, and MG190-177) (SEQ ID No. 501, 526, 528, 536, 577, 602, 653, and 659) were identified in the same samples in which the Cas12k effectors of MG64-6, MG64-7, MG64-13, MG64-18, MG64-29, MG64-51, and MG64-52 CASTs were identified (FIG. 30) and are likely associated with these CAST systems.

### Example 37-NLS Fusion with S15 of the MG190 Family is Necessary for Transposition (Prophetic)

The need for S15 with and without NLS tags in transposition experiments with MG64-6 or a Cas12k CAST of the MG64 or MG108 families is evaluated. NLS tags are fused to the N- and/or C-termini of S15 and tested in in vitro transposition experiments. Wheat Germ Extract in a Eukaryotic transcription/translation system can be used, which does not contain S15, to express MG64-1 CAST components and NLS-S15 constructs. CAST templates are amplified to contain a T7 promoter and a 40 bp Poly A tail for transcriptional stability of mRNA templates. Proteins are expressed from the dsDNA template via transcription/translation reactions, which are then used in an in vitro transposition reaction, as described previously.

### Example 38—in Cell Transposition with CAST and S15 of the MG190 Family (Prophetic)

NLS-tagged CAST proteins are expressed on high expression plasmids for transposition experiments in human cells. A targeting plasmid expresses the protein targeting complex, including S15, under control of a pCAG promoter. The targeting plasmid also contains a pU6 PolIII promoter driving transcription of a humanized sgRNA for in-cell targeted integration. A second donor plasmid containing DNA cargo flanked by the LE and RE terminal inverted repeats is transfected into cells. Cells are seeded 24 hours before lipid based transfection of the two plasmid system in 9 μg: 9 μg of targeting: donor plasmid. Cells are incubated for 72 hours at 37° C., then harvested by resuspension in 4 mL 1×PBS pH 7.2. 2 mL of resuspended cells are harvested for gDNA extraction and eluted in 200 μL of elution buffer. 5 μL extracted gDNA is assayed for transposition in 100 μl Q5 PCR reactions with primers specific for the target site. Amplified PCR reactions are visualized on a 2% agarose gel. Transpositions are predicted to transpose at 60-65 bp away from the PAM and are determined to be active by the presence of a single band for junction PCR amplification at the predicted size. PCR amplicons are Sanger sequenced and NGS sequenced for transposition profile analysis.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

TABLE 3
Sequence Listing of Protein and Nucleic Acid Sequences Referred to Herein

| | SEQ ID | | | | Other | |
|---|---|---|---|---|---|---|
| Category | NO: | Description | Type | Organism | Information | Sequence |
| MG64 | 264 | MG64-57 | protein | unknown | uncultivated | MSIITIQCRLVASESARRYLWQIMSEKNTPLVNELLKQVG |
| effectors | | effector | | | organism | KHSDFEKWLQAGKLPPGVVRSVCNLLASQECFAEQPKRF |
| | | | | | | YKSAIELTEYIYKSWLALQQRRQKQLDGKTHWLSMLKT |
| | | | | | | DAELVEISGCSLENIRAKALEILVRITTHPSNQPQQKKKSK |
| | | | | | | KGSNSKVSSKLSTTLFQAYDQTEDEPERCAITYLLKNNC |
| | | | | | | QVTEEEEDAKKFALRRRKKEKEIERLKEQLASRMPHGRD |
| | | | | | | LTGEQWLQTLLIAKTTVPQSEEEARSWQSSLLKKPSSIPFP |
| | | | | | | VQFTSKEDLIWSKNQNGRICIKFSGLGEHIFEVYCDRRQL |
| | | | | | | HWFQRFLEDQQIKRSGKDQYSSGLFTLCSGRLAWQENE |
| | | | | | | GRGEPWNVHCLTLYCSLDTRLWTIEGTETVSQDKAAEV |
| | | | | | | AKELTKMQDKGDLNPNQQNYIKRLSSTLTKINNPYPRPS |
| | | | | | | KPLYQGQTSILVGVSLGLEKPATAAVVDASTNTVLAYRS |
| | | | | | | LKQLLDENYKLLNRQRQQQQRNAHERHKAQKRSTPNQL |
| | | | | | | SESELGKHLDNLLAKAIITLAQTYQAGSIIVPNIKNVREGI |
| | | | | | | HSEIEAKAENKCPNFKEGQQKYAKQYRQTIHRWSYNRLI |
| | | | | | | DCIHSQAAKAKIPVEQAPQPIRGSPQEKARSIAIAAYHSRQ |
| | | | | | | NKS |
| MG64 | 265 | MG64-58 | protein | unknown | uncultivated | MSQITIQCRLVASASTRQKLWKLMAELNTPLINELLILAY |
| effectors | | effector | | | organism | QHPDFETWQHKGAIPAGIIKQLCEPLKTDARFVGQPGRFF |
| | | | | | | ASAIATVSYIYKSWVKVQKRLQLQIDGKTRWLEMLNSD |
| | | | | | | TELVEMAGVALDTLRATATEFLNQLNPQPTTEESPKKKG |
| | | | | | | KKTKKTQQLQGERSLSKILFDTYSDTEDVQTRCAISYLLK |
| | | | | | | NGCKIPNKEEDSKKFAQRRRKVEIQIQRLTDQLASRVPKG |
| | | | | | | RDLTGAKWLECLLEAVRKVPKNEAEAKSWQDSLLRQSS |
| | | | | | | SLPFPVAFESSEDMTWFRTLKLNNIPIKLWTLLLYIDYLT |
| | | | | | | VISFVRDSLQNEALWFKNLKINNIDVLKKVWMILLNINSF |
| | | | | | | AGILFLDGVLKKYQKRICVHENGLSDLMFEIYCDSRHLH |
| | | | | | | WFKRFLEDQQIKRSSKNQHSSSLFTIRSGRIAWKSTQGKG |
| | | | | | | KPWNVNRLMLYCCVDTRLWTAEGTNLVVEEKALEIAKT |
| | | | | | | ITRTKEKETKEQVQLNDKQLAYIKRKNATLTRINSPFPRP |
| | | | | | | SKPLYSGQSHILVGVSLGLEKPATLAVLNAITGKIIAYRSV |
| | | | | | | KQLLGKNYKLINRQRHEKLALSHQRKVAQTLAAPNEFG |
| | | | | | | DSELGEHIDRLLAKEIIAVAQKFNAGSIVVPNLDNMREQV |
| | | | | | | NSEIQAKAEEKCPESIEAQKKYASSYRRSVNQWSYRRLID |
| | | | | | | CITNQAAKAGIVIEENKQPIRASPQDKAKELALSAYNARK |
| | | | | | | KS |
| MG64 | 266 | MG64-59 | protein | unknown | uncultivated | MLLLNIFVHWLFDMSQITIQCRLIASVYIRQNLWKLMAE |
| effectors | | effector | | | organism | LNTPLINELLMQMRQHPDFETWRQKGKISTVVVKQLCEP |
| | | | | | | LKTDHRFTGQSARFYTSAVTTVSYIYKSWLALMKRTQY |
| | | | | | | QLEGKIRWLEMLNSDTELIEASGVSLDSLHTKAAEILLQL |
| | | | | | | APIDTAETQPVQRKKVKKGKKSQNSDSERNLSKNLFDAY |
| | | | | | | GNTEDNLTRCAISYLLKNGCKISDKEENPDKFAQRRRKL |
| | | | | | | EIQIQRLTEQLEARVPKGRDLTDTKWLETLYTAAQTVPE |
| | | | | | | NEAEAKLWQNILLRKSSKVPFPVAYETNEDMTWFKNQF |
| | | | | | | GRICVKFNGLSEHSFQVYCDSRQLHWFKRFLEDQQIKKD |
| | | | | | | SKNQHSSALFTLRSGCISWQEEEGKEEPWNIHHLTLSCCV |
| | | | | | | DTRLWTEEGTKLVKEEKAEEIAKTITQTKAKGDLNVQQQ |
| | | | | | | AHIKRKNSSLARINNPFPRPSKSLYKGQPHILLGVSLGLEK |
| | | | | | | PATVAVVDVVLGKVLTYRNIKQLLGDNYKLLNRQRQQK |
| | | | | | | QALSHARQIAQTQASPNQYGDSELGEYIDRLLAKEIIAIA |
| | | | | | | QKYSATSIVLPKLDGMREQVNSEIQAKAEQKCPESIEAQK |
| | | | | | | KYAKQYRRSVNQWSYGRLIENIKSQSAQAGIAIEEAKQLI |
| | | | | | | QGSPQEKAKELAFVAYNSRKKS |
| MG64 | 267 | MG64-61 | protein | unknown | uncultivated | MKKKDSETLITIQCRLVAEENTLRQLWELMADKYTPLIN |
| effectors | | effector | | | organism | ELLVQVGQHPDFDKWLKQGEVSKEAIETIKKSLITQEPFA |
| | | | | | | GQPGRFYKSAVILVEEIYKSWFALQQKRQRKIEGKERWL |
| | | | | | | KMFKSDIELQKKSQCSLDILRNKAKEILSSFSTKSNQPIKQ |
| | | | | | | ISKTQKRKNKEEVEYSTLFTSLFDLYDKTENCLSKYAIAY |
| | | | | | | LLKNDCQVSEIDEEQVQYAKRRRRKEIQVERLKEQLNSQ |
| | | | | | | KPKGRDLTGEKWLSTLKETTNQVPLDEFEAKSWQDSLLT |
| | | | | | | QTSNIPYPVDYETNTDLDWFIHSADDEIKRKIIIVWQIYFL |
| | | | | | | NELIKSGAYSLIKHLYFKMGCLPQKDVNCLNLQNKPGRI |
| | | | | | | FIKFNGLKKNVNNPEFYICCDSRQLHYYQRFCQDWQVW |
| | | | | | | HDKEINYSSALFVLRSARLLWQERKGKGAPWNVHRLILQ |
| | | | | | | CSIETRLWTKEEMELVRTEKIAQAEKTIRNMEQKTDLTK |
| | | | | | | KQLSRLKGERTLRQALEQPSPHCPSKSLYQGKSNIIVGVS |
| | | | | | | LGLEKPVTLAVVDVVKNKVLAYRSVKQLLAENYNLLNR |
| | | | | | | QRQQQQRLSHERHKAQKQNAPNSFGESELGQYVDRLLA |
| | | | | | | DAIIAIAQKYQAGSIVLPKLRDMREQISSEIQSRAENRCPG |
| | | | | | | YKEGQQKYAKEYRISVHRWSYGRLTESIKSQAVQAGISIE |
| | | | | | | IGTQPIRGSPQEKARDLALFSYQERQALLI |
| MG64 | 268 | MG64-60 | protein | unknown | uncultivated | MGFITIQCRLVASKAVRLLLWYLMASKNTPLVNELLKQA |
| effectors | | effector | | | organism | SQHDDFETWQRKGTVPEKSVQNLCKLLKTHPDFAGQPG |
| | | | | | | RFYTSASLMVIYIYQSWLALQQKRRQRLDGKQRWLSVV |
| | | | | | | KSDIELVRISNCSLQTIQDKAREILAQHNAQIGVDPDQTK |
| | | | | | | QKSKKKQPLEANGNLRRSLFQAYETTEDVLSQCAIVYLL |
| | | | | | | KNECQVREQEENPEAFAQRIHRKQKEIERLEAQLKSRLPK |
| | | | | | | GRDLTGEEFLQTLSTATGRVPADDIEQMLWQAKLLAKP |
| | | | | | | ATLPYPIIFGSQTDLRWSINKKGRICVSFNGLDKAIPELKE |
| | | | | | | NPLQIYCDQRQLPLFQRFLEDWQAYQTNKNTYPSGLSLL |
| | | | | | | KTGMLSWQEGTEKGEPWQANHLTLHCTINTRCLTVEGT |
| | | | | | | EQLRQDGIIRLRKQLAEGKKSEELTENQQDFIKRQKSTLT |
| | | | | | | RLENFSQLPKKSLYQGQPDILVGVSIGLAHPITISVVNART |
| | | | | | | GDVLTYRSTSQLLGDNYRLLNRQRQQQQRNTLKRHKNQ |
| | | | | | | VKGYTHQPSESELGQYVDRLLAESVIEVAQKFQASCIVLP |
| | | | | | | HTNNLREHLAAEIKAKAERKSDLVEVQDKYAKQYRINIH |
| | | | | | | RWSYDRLLSTIGAKADKMGLAIETTAQPHQGSPQEKARD |
| | | | | | | IAVAAYNFRQVALN* |
| MG64 | 269 | MG64-62 | protein | unknown | uncultivated | MAVSRPKPSEKKKKLDELYKVVCCWLCVDEATRKIIWE |
| effectors | | effector | | | organism | AMEKYTLLVNLLTERVAQLPEFEQWRQQGWVPEKAVM |
| | | | | | | GMCQALKQEEQFKGLPSRFYMSAQSMVKDTLDGWLKL |
| | | | | | | QQRLSRKINGKKRWLKLVEEDIELATVTDFDPEVIQNRA |
| | | | | | | KEILLELSQPENSEPQLSESLEEAPDESQQEDGSRDGQQSP |
| | | | | | | FSLLFDLYEIAEDCLSRRAIVHLLKNGGEVNEETEDLEKL |
| | | | | | | TQKLATKREAIQRLEKQLVSRLPKGRDPTGEQALQFLQE |
| | | | | | | AIQFPEHSNRYPSLFLFDIFYHYASLPMPYVQYAAYLLQA |
| | | | | | | ILAESKIVESEYLEWQGAMTERFHNAAKIPNSLPYPLLFG |
| | | | | | | STDDLYWSLESRGGSQQNSQPGKPCKGAKSRKRPSKRQ |
| | | | | | | RKKLKTRSEERICVRFKGLGNYVFKIYCDRRQLPIFRLFA |
| | | | | | | TDWLTYKELDKDSKYSLGLFALRSAKLIWKEDEQLLYK |
| | | | | | | KGKSKQANSGSTGISSELEALPPWKAHRLCLHCSIDPALL |
| | | | | | | TAEGTERVRSRKLSETKKTLDKAREKEQKLLVQISEVEL |
| | | | | | | AEEEKQKLENELAKRRSRILSYETSLIQLNNSPPRPSKVPY |
| | | | | | | QGEANITLGISFCRQELVGVAVVDLQSQQVLEYYSIWKL |
| | | | | | | LANQRVKKPRRDRSLLQLNLEKYRLVNRLRKLSKRNLA |
| | | | | | | YRREEQKQGQYVESKAESNLGQYLERLIAARIAQVALE |
| | | | | | | WRASSVTIPDLGDMREYIESTVQARAKQLFPNHREQQKN |
| | | | | | | YAKQFRIEGHRWSYAMLAEFICSRTLSEGITVETGRQSTT |
| | | | | | | GNLVNKALNVALSAHSLNLT* |
| MG64 | 270 | MG64-63 | protein | unknown | uncultivated | MSQSTIQCQLTANEATRSYLWALMAEKNTPLINELIGQV |
| effectors | | effector | | | organism | TKHSDFETWKLKGKIPSSVVSQLCQPLKTDPRFIGQPARF |
| | | | | | | YTSATHVVDYTFKSWLALQKRLSDTLQRKIRWSEMLKS |
| | | | | | | DPELVELSGCNLATIRAKAAEILAVATNKSQQGGTEPSQ |
| | | | | | | KTRKSKQPKDSAPGRTVSSLLFEAHRNTQDILSQCAICYL |
| | | | | | | LKNSCKVSDREEDPDKFAKRRRKVEIQIQRLQDQIEGRLP |
| | | | | | | KGRDLTGQSWLNTLAIATSTVPVDNAEAKRWQDKLLTQ |
| | | | | | | STSLPFPVSFETNEDLVWSNNQKGRISVRLNGLGEHTFQI |
| | | | | | | ACDQRQLPWFKRFLEDQQTKREGKNQHSSALFALRSGR |
| | | | | | | LAWQEGKEKGEPWKANRLILYCTVDTRLWTAEGTEQVR |
| | | | | | | NEKAIEIAQTLTRMKEKEGLSQTQQDYIKRLDSSLTRINSP |
| | | | | | | FDRPSQSLYQPLPHILVGICLGLEQPATVVVLDINTNQVL |
| | | | | | | THRTVRQLLGRNYALLLKRRREQQRTAHQRHKAQKRD |
| | | | | | | APRSLGESELGQYVDRLLAKATVELARTHRASSIVVPKL |
| | | | | | | GDMREIVQSEIQAKAEQKIPGSIEAQQKYAKQYRINVHR |
| | | | | | | WSYSRLIESIRAQAAKDGIALEEGRQPLQGSLEERAKGVA |
| | | | | | | IAAYQSRKK* |
| MG64 | 271 | MG64-64 | protein | unknown | uncultivated | CWLNVLKSDDELVELAGKPITDIYSQANEILSRFGDSVNR |
| effectors | | effector | | | organism | DSIFRTLFQRFKDSGDSPSRWAIAFLLKNGCKLPLEIEDVN |
| | | | | | | KFAKRRRKVEIQIERLTAQLEARLPKGRDLTGQKWLDTL |
| | | | | | | VVAATTMPESEIEAHRWQSELLKKPRTIPFPLIFETNEDLS |
| | | | | | | WFKNAKERLCMHLPGLREHSFEIYCDQRQLHWFKRFLE |
| | | | | | | DQKVKRQGKDQHSSGLFTLRSTRLSWLEGKGKGEPWNI |
| | | | | | | HRLALYCTVDTRLWTMEGTEQVRQEKVTEILKVIDHAK |
| | | | | | | TKSSLTKTQQSFVQRKESTLAKINNPFPRPSQQLYIGRSNI |
| | | | | | | LVGVSIGLQTPATIAVVDGITGKVLVYRSVKQLLGDRYN |
| | | | | | | LLNRHRQQQRQNTHRRHNAQKRFNSNQFETTDLGQYLD |
| | | | | | | ELLAQAIVKVAQKYQAGSIVVPKLENLREVIQSEIQTKAE |
| | | | | | | QKLPGFVELQKQYAKQYRINIHAWSYNRLIQSIQSQALQ |
| | | | | | | ANLCIEECVLDKQGNPQEKAKALAIAAYKARC* |
| MG64 | 272 | MG64-65 | protein | unknown | uncultivated | MSVITIQCRLVAEEETLRHLWELMAEKNTPLANEILERLA |
| effectors | | effector | | | organism | KHDDFKTWVEDAIVPKTVIKELCDFLKNQEPFAGQPGRF |
| | | | | | | YTSATTLVKYIYKSWLKLNRQLQRKIQGKERWLNMLKS |
| | | | | | | DAELEKESNSTIETIRQKASEILISLNAQRTKNIELKSIKPK |
| | | | | | | NQKQVQTVQSPKITSSVLFESYLQAEDTLTQCAIVYLLKN |
| | | | | | | NFKINSNEEDIDKYLKQRRKKEIEIERLKDQLKSRVPKGR |
| | | | | | | DLTGERWLSTLEQAVSTTFKDENEAKSWQAGLLRKSSN |
| | | | | | | LPFPVLYETNEDMKWEMNDKGRLFVSFNGLSKLKFEVF |
| | | | | | | CDKRHLYLFHRFLEDQETKRQGKNQHSSALFTLRSGRIA |
| | | | | | | WSEQAGKGQPWNLSRLHLFCSLDTLMLTSQGTQQVIEK |
| | | | | | | KITGVQDKLAKAQEKEKEEGGLNSQQQADVIRKKSTLA |
| | | | | | | KIKTPFSRPSKPLYQGKSHIIVGVSLGLEKLATIAVFDAVS |
| | | | | | | NKVLAYRSTKQLLGSKYNLLNRQRQQKKRLSQERHKSQ |
| | | | | | | KQFAPNNFGESALGQYVDRLLAKEIVAVAKTYGAGTIVV |
| | | | | | | PKLSDMREIIQSEVQAKAENKIPGFVEGQKNYAKSYRISV |
| | | | | | | HNWSYGRLIESINTQSAKVGIVIETGQQPTKGSPQEQARD |
| | | | | | | LALFAYQYRIA* |
| MG64 | 273 | MG64-66 | protein | unknown | uncultivated | MSVITIQCRLVADESTLRHLWELMAEKNTPLVNELLERL |
| effectors | | effector | | | organism | AKHSDFEAWLEDSKIPKTAIKELCDSLKTQESFAGQPGRF |
| | | | | | | YTSATTLVAYIYKSWLALQKRRQRKIEGKERWLEMLKS |
| | | | | | | DAELEQESNSSLEIIRTKATQLLCFFTAPHNSDTIQKSKAK |
| | | | | | | KSKKTKENNAGSSFSIKSGVLFETYRKSEDVLTKCALVY |
| | | | | | | LIKNNCHLSFLEEDPDKYVKLRRKKEIEIERLKDQLKSRV |
| | | | | | | PKGRDLTKEKWLETLEKAVNSVPQDENEAKSWQASLLR |
| | | | | | | KSSSVPFPVTYETNEDMHWEVNDQDRIFVSFSGLGKLKF |
| | | | | | | EVYCDQRHLHWFQRFVEDQETKRKSKNKHSSSLFTLRL |
| | | | | | | GRLSWLKQEEKGEPWHVNRLILFCSVDTRMWTVEGTQQ |
| | | | | | | VAIEKIADVEQNLIKAKEKGELNSNQQAFVTRQQSTLARI |
| | | | | | | NTPFPRPNKPLYEGKSHILVGVSLGLKNPANVAVFDAAN |
| | | | | | | NKVLAYRSVKQLLGDNYHLLNRQRQQKQRLSHERHKA |
| | | | | | | QKVFAPNDFGESELGLYLDRLLAKEIIAIALMYSAGSIVLP |
| | | | | | | KLGDMREIIHSEVQARAEKKIPGFKEGQQKYAKEYRKQV |
| | | | | | | HNWSYGQLIENIQSQAAKVGILIETGQQSIRGSPQEQARD |
| | | | | | | LALFAYQCRIASSI* |
| MG64 | 274 | MG64-67 | protein | unknown | uncultivated | MNLRLKAIALLRSWLHNQSGLLLKIFNSMSVITIQCRLVA |
| effectors | | effector | | | organism | DEKTLRHLWELMAEKNTPLVNELLDRLGKHTDFEAWV |
| | | | | | | QAGKVPKTTIKAMCDSLKTQEPFIGQPGRFYTSATDLLA |
| | | | | | | YICKSWLVLQKRRQRKIEGKERWLEMLKSDVKLEQESN |
| | | | | | | SSLELIRTIATEILNKFSASSTDGINQKSKGNKSKKFKKDK |
| | | | | | | ADEPISIKPGVLFEAYQKTEDILRRSALVYLIKNNCQVSLI |
| | | | | | | KEDPDKYAKMRRKKEIEIERLKEQLKSRVPKGRDLTGKK |
| | | | | | | WLETLDKAANSIPQDENEAKSWQASLLRKSSTVPFPVTY |
| | | | | | | ETNEDMYWEINDKGRIFVGFNGLSKLKFEVYCDQRHLP |
| | | | | | | WFQRFVEDQETKRKGKNQHSSGLFTLRSGRLSWFKQEG |
| | | | | | | KGEPWSVNRLILFCSVDTRMWTVEGTQQVAIEKIADVEQ |
| | | | | | | NLTKAKEKGELNSNQQAFVTRQQSTLARINTPFPRPSKAL |
| | | | | | | YEGKSYILVGVSLGLENPATVAVFDAANNKVLAYRSVK |
| | | | | | | QLLGNNYNLLNRQQQQKQRLSHDRHKAQKQFALNDFG |
| | | | | | | ESELGQYVDRLLAKEIVAIALTYFAGSIVLPKLGDMREIIQ |
| | | | | | | SEVQARAEKKIPGFKEGQQKYAKDYKRSIHNWSYGRLIE |
| | | | | | | NIQSQAAKPGILIETGQQSIRGSPQEQARDLALFAYQSRIA |
| | | | | | | SSI* |
| MG64 | 275 | MG64-68 | protein | unknown | uncultivated | MSVITIQCRLVADDKTLRHLWELMAEKNTPLVNELLDRL |
| effectors | | effector | | | organism | GKHTDFEAWVQAGKVPKTTIKALCDSLKTQEPFIGQPGR |
| | | | | | | FYTSATILVAYIYKSWLALHKRRQRKIEGKERWLEMLKS |
| | | | | | | DVELEQESHSSLELIRTRATEILSKFSASSTDGINQKSKGK |
| | | | | | | KSKKVKKDKADEPISIKPGVLFEAYQKTEDILRRSALVYL |
| | | | | | | IKNNCQVNLAEEDPDKYAKMRRKKEIEIERLKEQLKSRV |
| | | | | | | PKGRDLTGKKWLETLEKAVNSIPQDENEAKSWQASLLR |
| | | | | | | KPSTVPFPVAYETNEDMHWEISDKGRIFVSFNGLSKLKFE |
| | | | | | | VYCDQRHLPWFQRFVEDQETKRKGKNQHSSGLFTLRSG |
| | | | | | | RLSWLKQEGKGEPWSVNRLILFCSVDTRMWTVEGTQQV |
| | | | | | | AIEKIGDVEQSLTKAKEKGELNSNQQAFVTRQQSTLAKIN |
| | | | | | | TPFPRPSKPLYEGKSHILVGVSLGLENPATVAVFDAGNNK |
| | | | | | | VLAYRSVKQLLGNNYNLLNRQQQQKQRLSHDRHKAQK |
| | | | | | | DFTRNDFGESELGQHIDRLLAKEIVAIAVTYFAGSIVLPKL |
| | | | | | | GDMREIIQSEVQARAEKKIPGFKEGQQKYAKEYRKQVH |
| | | | | | | NWSYGRLIENIQSQAAKVGILIETGQQPIRGSPQEQARDL |
| | | | | | | ALFAYQCRIASSI* |
| MG64 | 276 | MG64-69 | protein | unknown | uncultivated | MSSSEKPIPFEKAMQTILALLTIDQDSRQYLWNMCIAYTL |
| effectors | | effector | | | organism | LINEIFQRVAQHPKFLEWLNQGKLPIGIVKLICKKLEEDEF |
| | | | | | | TGLPKRVYVSAILLVSHTFKAYFAMQSDLQLKLKGKQR |
| | | | | | | WLEVMETDLELAKNTDITSDLIRVRAAEILTEIEAQRSLSS |
| | | | | | | NQQDRQSEPQEQNNSTSLASNSLMSFLFKKWDIAESSLE |
| | | | | | | RRAIAHLLRNDCQVNSEEEDPDKLSLRLERKEIEIQRLED |
| | | | | | | RLKSRLPKGRDPLGERSQQFLEEAIAFAEHYSYVIQNWF |
| | | | | | | WLKWHQTILSRHPADAERLNYWTLFLIYYRWNNAAEFK |
| | | | | | | AWEQDLSRRAANLQTSFSSLPYPLLFESTDDLYWSWEKE |
| | | | | | | EIKDQTRAQNQSTSKNCKKDLKQKRHRTRKRKRQLEKRI |
| | | | | | | CVSFKSKGLKCFRFKLYCDRRQLPVFRQFVTDFETYRAL |
| | | | | | | PKEDKFSIGLFALRSAHLLWKEDKQGLERKKHWRLQNL |
| | | | | | | WLKWYCAMLHNSSLEGEIMDSWCRSLIYLEISIRLPWKT |
| | | | | | | HRLHLQCTFDPRLLLAEGTEAVRQEKLTLVRKKLDNLEK |
| | | | | | | SLEISEKQIQSCQNSEAQSIDNLTESEELAEPPSEEEKNQK |
| | | | | | | LINRQKARIRILSTLDRLENSTPTRPSMISYNGNCDIVASV |
| | | | | | | CFSRLNVIGIAVINTRSQTVLAYQNLRTLLTNQRTEVLER |
| | | | | | | RAVKITSRKGRTIVKHSAEHPAKFKARRKIKVQRKARRS |
| | | | | | | VVQLSLEQYRLFNRWQNEQRKNLSGRKEEQKRGLYAES |
| | | | | | | RKESNQAQYLNRLIARRLIQLCQKWQVGSIILPDFGDLRE |
| | | | | | | SVECEIQARAKRKFPDDNVKLQKQYAKHLRMAFHRWN |
| | | | | | | HKGLSQAICSCAASMGIPVKTGQQPSQGTLREKALAMAI |
| | | | | | | AV* |
| MG64 | 277 | MG64-70 | protein | unknown | uncultivated | MVVSMFTIQSRLCASEETRRYFWELMEKHTLLVNELLEK |
| effectors | | effector | | | organism | IAQHPQFQEWQKKGAISGNTVRGILAPLKENSGYVGLPG |
| | | | | | | RFYTSAELISCYTYKSWLALQKERQLQLLGKKRWLQAV |
| | | | | | | ESELELIATTDFNPDEIRVKAHEIQKKALDKLNKESKKQK |
| | | | | | | ALISILLDMHDGTAEAPLSRRAINHLLINNLKINEKEQNLD |
| | | | | | | QLSERLDKKRKEIERLEEQLTSRLPKGRDPTEQRYLENLC |
| | | | | | | HVTALPELSDDPEKLAAELETLTVQKQLPLLKELPYPIQF |
| | | | | | | GSSGDLYWSVETQEKKHCQRPQQRICVRFKGAKDHTFKI |
| | | | | | | QCDCRQLSIFRQFLIDYQTYQELPYEERFSQGLFALRSAC |
| | | | | | | LIWRKDDSKHGSNKKRTTDNQEAQLKPWNTHRLYLHCT |
| | | | | | | VDRQMLTAEGTEQVREAKKKEVIKTLKNKEKLQELELE |
| | | | | | | QLGLTKTQIESVGRKRSTLTYLEKNSPPPRPNAKPYQGQP |
| | | | | | | HIVVGVSFSRHEPVAIAVVDVEKEEVLERQSAKELLNRG |
| | | | | | | EAQYIWRNGKKEPLIKDGTEQRHPNGGKLYIRKGKRVRR |
| | | | | | | KPHRLVQQLHQRHQQNSRRRSEEQKQDRYRSSNSDSDL |
| | | | | | | GLYVERLIASKIVELALQRKAGTIAIPQLKGIRESVESDIR |
| | | | | | | ARAERLFPNEKERQKEYGKGYRASFHSWSYSRLSDCIKE |
| | | | | | | CASSEGIAVVIRQQPSGIELEQKAIAIALSSYNVKTS* |
| MG64 | 278 | MG64-71 | protein | unknown | uncultivated | MSIITIQCRLVANESTRRYLWQIMAEKNTPLVNELLKQV |
| effectors | | effector | | | organism | GKHPDFEKWLQAGKLPPGVVRPLCNSLVSQECFANQPK |
| | | | | | | RFYKSAIELTEYIYKSWLALQQRRQKQVDGKTRWLSML |
| | | | | | | KSDAELVEISGCSLENIRAKALEILVRITTYSSNQQQQKK |
| | | | | | | KSKKVSNSKASSKLSTTLFETYDQTEDKLERCAITYLLKN |
| | | | | | | NCQVTEEEEDEKKFALRRRKKEKEIERLKEQLASRMPHG |
| | | | | | | RDLTGEQWLQTLLIATTTVPESEEEARSWQSSLLKKPSSIP |
| | | | | | | FPVEFTSKEDLVWSKNQNGRICVKFSGLGEHIFEVYCDR |
| | | | | | | RQLHWFQRFLEDQQIKRSGKDHYSSGLFTLCSGCLAWQE |
| | | | | | | NEGRGVRGSAATAALWNVHRLTLYCSLDTRLWTIEGTE |
| | | | | | | TVSQDKAAEVAKELTKMQEKGDLNPNQQNYVKRLSSTL |
| | | | | | | TKINNPYPRPSKPLYQGQTSILVGVSLGLEKPATAAVVDA |
| | | | | | | STNTVLAYRSLKQLLSENYKLLNRQRQQQQRNAHERHK |
| | | | | | | AQKRSTPNQLSESELGKHLDNLLAKSIITLAQTYQAGSIV |
| | | | | | | VPNIKNVREVIHSEIEAKAENKCPNFKEGQQKYAKQYRQ |
| | | | | | | NIHRWSYSRLIDCIHTQAAKAKIPLEQGPQPIRGSPQEKAR |
| | | | | | | SLAIAAYHSRQNKS* |
| MG64 | 279 | MG64-72 | protein | unknown | uncultivated | MSKSTFLFRLDALGDASQIREENRRYYWELMTQQHTPLI |
| effectors | | effector | | | organism | NQLNLKVAQHPNFADWQINNAVNADELKSLWRSFKEHP |
| | | | | | | QFETMPERAFVSARLVVGDTYESWLALQGDRQEELAKL |
| | | | | | | SNSLEVLKTDAELVAISGCSLDAICSKAREILSQANTQVA |
| | | | | | | GESKKKSKKAINGGIYKVLYEKHRIADDPLKKCAAAYLI |
| | | | | | | KNRFEIKGDETEEDIEHLKNRIHCKEKQIELLLKQLQSRLP |
| | | | | | | KGRPWVGDGILDEIKSIGVVEDEAEWNLVESALLKQQTF |
| | | | | | | LPHPMLFHSSDDLIWFEHERPPSQDSTTEDERKPNAESSR |
| | | | | | | RVCVRFKSFDEKYAFEISGDRRHLHILQQALRERMIYDSD |
| | | | | | | IDGNTSKLFLVRSATLIWKEYRKNENRIIRRRKAANKRAK |
| | | | | | | RIVQSIDKAPEIQSAPAFYNPEFPWNRYQLFLHCTVETEFL |
| | | | | | | SQEGTELVIQQQRKPIIKALQTLEERMAESENKGESTQTR |
| | | | | | | KANHSRKTGTLRRIDSYDNNYDRPSKPLYAGIPHILTGVA |
| | | | | | | LGSGGLVTVTIVDATSGKILGCRGLKALLGDNYRLVNRR |
| | | | | | | QFQRQLNLRRRTERQKRGASSQSGESNLGDAIDQHTANA |
| | | | | | | VLDFAKKHHSGCIVLPDMKDYRVRQQSEIAALAERECSG |
| | | | | | | WKGIEKRFAKAQNMKIHAWSYGRLMEYIRNQAHKGGIL |
| | | | | | | VKIGQQPLYGSSQEQAGKMAIDAYQNKTVPKNSP* |
| MG64 | 280 | MG64-73 | protein | unknown | uncultivated | MSQITVQCRLVAPEPVRQTLWELMANLNTPFINELLQQT |
| effectors | | effector | | | organism | AQHPDFGQWRQQGRLKATIIKQLGNQLKEDSRYLGQPG |
| | | | | | | RFYTSGISLVEYVFKSWLKLQQRLQQKLYRKRRWLEVL |
| | | | | | | KSDEELIIETGVNLDVIRNKATKIIQAHQSEDKLENTLFDA |
| | | | | | | YKEEQDLLTRNALRYLLKNRCQLPKAEEDAQKFARLRR |
| | | | | | | KTEISTERLQDQLDSRLPQGRDLSNNTWLETLAIARNTDP |
| | | | | | | KDQKEARSWQDKLLTQASSIPFPVTYETNEDLTWSKSKK |
| | | | | | | GRLCVQFSGLSDLIFQIYCDQRQLKWFQRFYEDQQVKKN |
| | | | | | | GKDQHSSALFTLRSGRILWQEGTGKGHPWNIHRLTLQCT |
| | | | | | | LDTRLWTAEGTEQIKQEKAEEIAKVLTRMNEKGELNKN |
| | | | | | | QQAFIKRQQTTLGRLGNAFPRPSKPLYQGQSNIIAGVSMG |
| | | | | | | LEKPATVAIIDVITGKTLTIRNTKQLLGKNYFLLNRQRILK |
| | | | | | | QSQSHQRDVAQRKEAFNRFGDSQLGEYIDRLLAGAIVQL |
| | | | | | | AKTYQAGSIAIPKLEDIRESVQAEIQARAEEKIPNCLEAQA |
| | | | | | | KYAKQYRVSVHQWSYGRLIENIQAQADKVGIVIEEAEQV |
| | | | | | | IRASPQQQARELAINAYAARSLA* |
| MG64 | 281 | MG64-74 | protein | unknown | uncultivated | MTMKAIQFDVIARKDPKYRRNREKPTDQLEEEALWEVV |
| effectors | | effector | | | organism | QASCHHTLLVIEILKQMEQPSAFPARIKKLKQPQADGILP |
| | | | | | | DIKQEEEWLETEIEKACKSLKEQAEFQNLPGRIYSSAIHQS |
| | | | | | | LQPLKGWLENQWQLLLRLSGKNRFLAVVETDADLAQAS |
| | | | | | | DFSWSDIQARAQAILQQTQEAIAAKAKDETAAKDTKQLL |
| | | | | | | KSLLKQYDATSNILARRAIIHLLRNKFKVRRKPENPKRLQ |
| | | | | | | ALLEGKRVEIERLEAQVPRLPRLRNLVPEQAYDTALEELT |
| | | | | | | TYPLSDVAVSERVAWLLHYRVLICFFLIYITSAEKNLQLA |
| | | | | | | DCLLHLVRIEIERGEAQFYQWHDGVPAKINQFLTIPKSLP |
| | | | | | | YPIYFGGDNLRSWQLNQEGKICFKLNGLGDYLFEVRCDR |
| | | | | | | RQLGIVKYFLQDWQTQNKNKNEYSGGLTLLRSAELLVK |
| | | | | | | PKLGKQNAKLPPIHDRQAVVTAYKLSLHCTYDTDYLTH |
| | | | | | | QGLECVRQRKIANQLKGLTDKKAKLTKQQEQLQQLEQE |
| | | | | | | MQQEQIGTSAKRSKRHAQRLKQIEQLKQSISKLQAAIQAE |
| | | | | | | LERPRPKLERLQQSQLFQRADRPLYAGVAHLFVGVCLDL |
| | | | | | | DQHLVVTIVDAMRHKVLSKRTGKQIMGEHYPLLQRYRR |
| | | | | | | LKQQHPKQRRQDQKVGRHNHLSETGLGEQVACAIANGL |
| | | | | | | LSLAQQYKVSTIVLPETKGWRERLYSQLVARAKIKCNGS |
| | | | | | | KKAMARYTKAYGKRLHQWDYNRLSRAIETEAQTVGVT |
| | | | | | | VIFQRLEFQANAEQDNQPADEADEQDNQRVNPFELALQI |
| | | | | | | AIAAYDSLQA* |
| MG64 | 282 | MG64-75 | protein | unknown | uncultivated | MSIITIHCRLVASEPIRRHLWHLMTESNTPLINDLLNQVSQ |
| effectors | | effector | | | organism | HPDFETWQRRGTVPEKTVKELCEPLKAIYPGQPARFYAS |
| | | | | | | AILMVTYTYESWLALQQNRRRRLDGKQKWLNVVKSDA |
| | | | | | | ALLELSSTTLEAIQERAQTVLKQLNVEPETQAASNPKKRK |
| | | | | | | AAQQQTQSANKASPMTLLFEAYDAIDETLSRCAIAYLIK |
| | | | | | | NGCKIPETEEDPEKFAHRLRRKQKEIEQLEAQLQARLPKG |
| | | | | | | RDLTGEEFMETLAIATQQISESVAQAREWQAKLLSRSVC |
| | | | | | | LPYPIIYGSSTDVRWGTTAKGRIAVSFNGIDKYLKATDPD |
| | | | | | | IEAWFKTSQEPPFRLYCDQRQLPFFQRFLRDWQAYQAEK |
| | | | | | | DTYPAGLLTLSSAMLAWREGEGKGDPWNVNHLALYCSF |
| | | | | | | DTRLMTAEGTLEVQREKADKALKNLTHAKPDPRNQSTL |
| | | | | | | NRLKNLPDRPSKKPYQGKPEILVGLSIGLANPVTVAVVN |
| | | | | | | GTTGDALIYRTPHTLLGNHYHLFNRHRQQQQQNALQRH |
| | | | | | | KNQRRGVAYQPSESELGQYVDRLLAKAIIQLAQTYQAGS |
| | | | | | | IVVPNLTHLRELLASEITARAEQKASLVEAQNKYAKEYR |
| | | | | | | QTIHRWSYNRLIEAIRSKAQQLGITVESGFQPLQGNSQEQ |
| | | | | | | AKDMAIAAYHARAINTK* |
| MG64 | 283 | MG64-76 | protein | unknown | uncultivated | MTVITIQCRLVAKEETLRHLWELMTQKNTPLINEVLEQIG |
| effectors | | effector | | | organism | KHPELEECIQKGKLPIGLVKTLCNSLKTDLQFSGQPGRFY |
| | | | | | | SSAISLVDYIYKSWLALQQGRQRKIEAKERWLSILISDDE |
| | | | | | | LEKACNSSLDVIRAKATKLLTQNGPPSNLNHNQPTESKK |
| | | | | | | GQKTKKGKADKPPRRLFNLLLDAYENTTDPLERCSLAYL |
| | | | | | | LKNDCQVSELEEDPTEFALRRRAKEIEIERLKEQLESRLPK |
| | | | | | | GRDLTGEKWLEALETAIHNISKDEDEAKAWEAALLRKSS |
| | | | | | | SVPFPVAYESNEDMNWFKNDQGRICVRFNGLGKHTFEV |
| | | | | | | WCDQRQLHLFQRFLEDQQTKRDSKDQHSSSLFTLRAGRI |
| | | | | | | CWLERQGKGLMWNRHRLILYCCVDTRLLTAQGTQQVK |
| | | | | | | SEKAAKIAKILTKTKDKEELDDKQQAFVKRQQSTLDRIN |
| | | | | | | TPFRRPSKPLYQGQPSILIGVSLGLKKPATVAVVDAKEGK |
| | | | | | | VLTYRSVKQLLGENYKLLNRQRQQQQSLSHERHKAQKR |
| | | | | | | DTPNEFGESELGQYVDRLLAHEIVAIAKAYQAGSIVLPRL |
| | | | | | | GDMREIVSSEVQARAEQKVPGYKEGQQKYAKQYRVSV |
| | | | | | | HRWSYGRLIEIIQSLAAKTGIAIEVGQQSIRGSPQEKARVL |
| | | | | | | ALSAYSSRITCMN* |
| MG64 | 284 | MG64-77 | protein | unknown | uncultivated | MNMLMFTIQCRLCASEQTRRYWWESMEKYTLLVNELLE |
| effectors | | effector | | | organism | KIAQHPQFQEWQKKGDISREAVRKILNPLKESLQYAGLP |
| | | | | | | KRFYTSAELISCYTYQSWLALQQQRQLRLLGKQRWSEA |
| | | | | | | VESEFELSATTDFSPDKIRVKAHTILNKAIQKLNQQGKKP |
| | | | | | | KHLMDILLKKHQKTAKDSLSRRAINHLLINNLKVSQEEQ |
| | | | | | | NLNELSERLDKKRVEIKRLEEQLKSRLPKGRDPTRQRYL |
| | | | | | | QILSHISVLPDLRDDPQKLEAELDRLTIQQQLPLFNELPYPI |
| | | | | | | LFYSSSDLYWSVQPQDTSHPSEPENGSHPELPKSKKHQKP |
| | | | | | | HGKRPTERISVKFKGVQSKEAKPNTGQSKEAKDHTFKIQ |
| | | | | | | CDRRQLPLFRRFLIDYQTYDNLPEEERFSEGLFTLRSACLI |
| | | | | | | WRKDESRHRSTKKIGTDQPEDQLKPWNTHHLYLHCTVD |
| | | | | | | RRLLTAEGTEQVRAEKKQATLKELKGKDKLEQTELDEL |
| | | | | | | GLTKNQISSIKRKCSTLNRLENHSLPPRPNSVPYQGQPGIT |
| | | | | | | VGVSFSRHQPVAIAVVDVNKQEVLERQSAKELLNRGKA |
| | | | | | | QYLWRNGLKESLTRDGTERRHPNGGKLLIRKGKRVRWK |
| | | | | | | PYRLVEQLHRRHQHHSQQRAKQQQQNRYQESNVDSNL |
| | | | | | | GLYVDRLIASRIVKLALQRKVGTIVIPQLKGIRESVESDIR |
| | | | | | | AQAERKFPNEKERQKEYAKHYRASFHSWSYARLAKCIK |
| | | | | | | ECGAREGIAVVERKHSSQGDLEQKAIAIALSSYNVKTS* |
| MG64 | 285 | MG64-78 | protein | unknown | uncultivated | GRDPTRQRYLQILDHISVLPELQDDPQKLEAELDRLTQLP |
| effectors | | effector | | | organism | LYNELPYPILFHSSGDLYWSIESQDTSNPCSPENGIHPELP |
| | | | | | | KIKKRHKKHCKQPTERICVQFKGTESEEAKPKKSQSEVA |
| | | | | | | KDYTFKIQCERRQLPVFRQFLIDYQTHKQLPEEERFSEGL |
| | | | | | | FALRSACLIWRKDDKRHRSKKTRTADQPEESPKPWHTHR |
| | | | | | | LYLHCTVGRPLITAEGTEQVRQEKKREVIEELQGRDQLEE |
| | | | | | | SQLQELGLNKKQIVYVKRRRSTLNRLKNNSPPRPSIQPYQ |
| | | | | | | GQPHIVIGVSFSRHQPVAIAVVDVNKEEVLECQSAKELLN |
| | | | | | | RGEAQYLWRNGKKELLIRDGTERRHPNGGKLYIRKGKRI |
| | | | | | | RWKPYRLVEQLHQRYQHHSRQRAKQQQQNRYQQSDSD |
| | | | | | | SNLGLYVDRLIAAQIVELALQRKAGTIVIPQLRGIGESVES |
| | | | | | | DIRAQAQRLFPNEKERQKKYALHYRASFHCWSYARLSQ |
| | | | | | | CIRECATREGIAVVERKQSSQGDLEQKAIAIALSLCNVKS |
| | | | | | | S* |
| MG64 | 286 | MG64-79 | protein | unknown | uncultivated | MSQITIQCRLVASEPTRQHLWKLMADTNTPLINELLKQV |
| effectors | | effector | | | organism | GQHPDFESWRHKGKLPAGIVKQLCQPLRTDPRFIGQPGR |
| | | | | | | FYMSAITVVDYIYKAWLALQKRLQYQLEGKTRWLEMLK |
| | | | | | | SDTELIEATGCTLDILRIKATEILAQHAAFAPDPTQTPTTK |
| | | | | | | GKKGKKRRTANTNHNLSEALFEAYRETDDILTRACICYL |
| | | | | | | LKNGCKVTTKEEDPEKFNLRRRKVEIRVKRLTEQLASRM |
| | | | | | | PKGRDLTSETWLETLAIATSHVPQNEDEAKSWQASLLRQ |
| | | | | | | SSSVPFPVAFETNEDLRWSKNQKGRLCVEFNGLSEHTFE |
| | | | | | | VYCDKRQLHWFQRFLEDSLIKRDSKNQHSSSLFTLRSGRI |
| | | | | | | AWQEGEGKGDPWNVHRLTLYCSIDTRLWTHEGTEQVR |
| | | | | | | DEKAAEIAKTLTAMKEKGDLNEKQQAFIKRKNSTLARIN |
| | | | | | | NSFPRPSNPLYQGQSNVLVGVSLGLEKPATAAVVDAMT |
| | | | | | | GKVLTYRSIRQLLGENYKLLNRQRQEQHQNSSKRHNAQ |
| | | | | | | SQGAPNQFGESNLGEYVDRLLAKAIIALAKTYHAGSIVLP |
| | | | | | | KLKDVRESIQSEVQARAEQKCPELIEAQKNYAKQYRSSA |
| | | | | | | HSWSYGRLIESIQSQAAQAGIIVEEARQALVGSPQDKAKK |
| | | | | | | LAIVAYTSRLQAII* |
| MG64 | 287 | MG64-80 | protein | unknown | uncultivated | MSEITIQCRLMTSEETRRYLWQLTAPKNTPLVNELLKLVS |
| effectors | | effector | | | organism | QHPDFEAWRRRGTLPGNAVKQLCEPLRQDPRFVGQPGR |
| | | | | | | FYSSAIRIVQQTYKAWIASIRAKQASLDGKKRWVETVES |
| | | | | | | DAQLAEMGNFSPEVIYTKAREILEQVSTVLLSPTTPAKQP |
| | | | | | | KRAKKSKKNKENSSVINTLFELFNTTEDLLSRRAIIHLLRH |
| | | | | | | GWQVNEQEEDPEKLSQLLARKRQEIKRLEEQLQARCPRG |
| | | | | | | RACTEEEAQERVRRAISLPEHPGLMFSLYLSVALLCAPTP |
| | | | | | | SSRQLILTWLLKRICQFEEARVHSEFLDWEENFPSNRLSL |
| | | | | | | MRSPKNLPYPILYGPEDLNKWCRNEKGRICLSFNGLSEYT |
| | | | | | | FELQCDRRQLSLFELFMEDWQTLRAKENQKQYSGSKLLL |
| | | | | | | REATLFWQEPTQKIIKKKFNRQPDTQTEHPLENEATQQR |
| | | | | | | CGRNSDPWNKYRLTLHCTIDTKLLTLEGTEQIRQEKLAK |
| | | | | | | RSKELESRSQKSKLDEDQALNRERAKQECLQNAKYS* |
| MG64 | 288 | MG64-81 | protein | unknown | uncultivated | MFTIQCRLCTSEETRRDIWQWMEKYTLLVNELLEKIAQH |
| effectors | | effector | | | organism | PQFPKWHKKGNITRKAVGEILNPLKENPQYAGLPSRFYT |
| | | | | | | SAELISCDTYKSWLALQQQRQLRLLGKQRWLEAVETELE |
| | | | | | | LSATTDFDPDKIRAKAHSIREEALQKLNQQGKKPKDLMD |
| | | | | | | ILFKKHQKTVKDSLSRRAINHLLINNLKVSQEEQNLNELS |
| | | | | | | ERLDKKRVEIKRLSEQLKSRLPKGRDPTRQRYLQILDHIS |
| | | | | | | VLPELQDDPQKLEAELDRLTQLPLYNELPYPILFHSSGDL |
| | | | | | | YWSIQSQDTSNPSSPENGIHPELPKIKKRHKKHCKQPKERI |
| | | | | | | CVQFKGTDSEEAKPKKSQSEGAKDYTFKIQCDRRQLPVF |
| | | | | | | RQFLIDYQTHKQLPEEERFSEGLFALRSACLIWRKDDNRH |
| | | | | | | CSKKKRTADQQEESPKPWHTHRLYLHCTVGRPLITAEGT |
| | | | | | | EQVREEKKREAIEELQGRDQLEESQLQELGLNKKQIVYV |
| | | | | | | KRRRSTLNRLKNNSPPRPSIQPYQGQPHIAIGVSFSRHEPV |
| | | | | | | AIAVVDVNKEEVLECQSAKELLNRGEAQYISRKGSKELLI |
| | | | | | | RDGTERRHPNGGKLYIRKGKRIRWKPYRLVEQLHRRHQ |
| | | | | | | HHSRQRAKQQQQNRYQQSDSDSNLGLYVDRLIAAQIVN |
| | | | | | | LALQRKAGTIAIPQLRGIGESVESDIRAQAERLFPNEKERQ |
| | | | | | | KKYALHYRASFHRWSYGRLSQCIRECATREGIAVVEKKQ |
| | | | | | | SSQGDLEQKAIAIAISLCNVKSSWLKHSSICTLTR* |
| MG64 | 289 | MG64-82 | protein | unknown | uncultivated | MFNAYYATDDTLQKCAIAYLIKNKRQVNDKEEDLKKLT |
| effectors | | effector | | | organism | RLIKQKKKKIERLQKQLESRLPKGRQWLGNDFIDNLKAF |
| | | | | | | GIPESEVEWFSLQSALLGEHNFIPYPILFGSSDDLIWSKQL |
| | | | | | | KISNNSNLIKEKLESEKSRERICVQFKGLKEVVFEISCDRR |
| | | | | | | QLPLFQQFLKDWTIYSQNPKEHTSSLFLIRSATLIWKDTK |
| | | | | | | KTKNRQKRWNKNKTVNQKCIDPQQEELKQLVQAGINEE |
| | | | | | | EQPWNRYQLFLHCTVATEFLSKEGTQQLGQKKQELALK |
| | | | | | | AIATLEQKILELEKEGKSTKNDRESFSRKQGTVRRLNNLD |
| | | | | | | NPFERPSRPLYQAQPNILLGVSLGSSKLATATVVDVTTEK |
| | | | | | | VLECQGVRCLLGDNYKLLTRKQYLHEMHSHLRSKAQKR |
| | | | | | | GAKNLLREAKLGEHIDRLIATAIIALARKYQASTIVLPDM |
| | | | | | | KDYTEKKQSEIEAFAEQECSGWKCVEKRFTKAQSVKLH |
| | | | | | | RWSYSRLSKIICQQASKVGIAVEIGQQPIHGSSQEQSRAM |
| | | | | | | AIETYHSRKNSLKSKNLRS* |
| MG64 | 290 | MG64-83 | protein | unknown | uncultivated | MSTITIQCRLVASEPTRQQLWTLMAERNTPLINELLAQIS |
| effectors | | effector | | | organism | QHPDFDTWRQQAKLKPVIVKQLCQPLKSDPRFSGQPGRF |
| | | | | | | YDSAIALVEYIYKSWLKIQQRLQRKLEGQSRWLEMLKSD |
| | | | | | | EELVQMSNCTLEVIRAKAALVLTPLASQNQSTQPTNTKS |
| | | | | | | KKRKKPQASNSNRSVSKALFEAYANTEDILTKSALCYLL |
| | | | | | | KNGCKISDKEEDPEKFAKRCRQTEIKISRLTEQIASRIPKG |
| | | | | | | RDLTGEKWLETLITATSTAPESETQARSWQDRLLTQSKSI |
| | | | | | | PFPVAYLSNVGLTWSKKEKEENGKNLSKYRKGGRKNQE |
| | | | | | | GRLCVKFNGLGEHIFEIYCDQRQFQWFQRFYEDGQIKKE |
| | | | | | | SKDQHSSALFTLRSAQIVWQEGFGKGEPWNIHRLALYCT |
| | | | | | | LDTRFWTTEGTEQIRQEKIITLEKTLLRIKPELTLELFFRSH |
| | | | | | | LILKFLSIWCVITTHKTVEMLKEGDLSEQRKNSRAFRKST |
| | | | | | | ESSLQKINTPFPRPSQPLYQGQPHILVSVALGLDKPATAA |
| | | | | | | VIDGTTGKAIAYRSIKQLLGDNYKLLNRQRQQKRSYSHQ |
| | | | | | | RHKAQKKAASNQVGGSDLGQYLDRLLAQAIVKLAQTH |
| | | | | | | QAGSIVLPKLGDMREVVQSEIQARAEQKIPGYIEAQEKY |
| | | | | | | AKQYRVNIHHWSYGRLMDNIKAQSSKVGIFIEEGEQPIRG |
| | | | | | | SPQEKAKNMGISAYHARSNS* |
| MG64 | 291 | MG64-84 | protein | unknown | uncultivated | MSDVLLVSDRDTEGELKAMRTIRLVLLADAETRQHFWC |
| effectors | | effector | | | organism | LSLVHTFLVNELFQTLPKHKDFPKWQRQRRVSVESVKKL |
| | | | | | | IDQLKKKDFLGTPPQIFSSAIAIVCSTFKAYFALQQKCQLK |
| | | | | | | LDGMRRWLRTVESDLELARTTAFSAETICSRARALLVEL |
| | | | | | | EAQRHHNRHEPQLQASHEPQPLPAPTSLMSDLFKRLESA |
| | | | | | | EDPLERRAVVHLLKNACAVNVAEEDPDKLALRLEKKKI |
| | | | | | | QIQRLEKQMASRLPVGRDPTGDRAHQAIEAAISFAEHTPV |
| | | | | | | SFWLKWHGVLLTNRSVNALSLELWVLGYLYDCLNADA |
| | | | | | | EFEAWEQALPSRMANLSTQWAALPYPLIFDSTDDLYWSR |
| | | | | | | APEPPLKPPCSGKKAAGTMKPLHHKRKRSRTRKRQKKLT |
| | | | | | | ERIMVRFKGKGLSHCRFKVGVDRRQLPIVQQMVDDEQA |
| | | | | | | HKARAADDKFSLGLFALRSACLRWDVDPQKLHTKQHW |
| | | | | | | KLQSLWLKWFCTLPNGTLMQQSELDLWFISLFYLALSKS |
| | | | | | | IPWQTHRLSLHCTIDPRLLTAEGTEAVRQEKLAQTLERIA |
| | | | | | | QVEKKLQLAEQQTEQQAHEGGETLATAGEITQLDEDLDE |
| | | | | | | ESVAQKQESVAQKRQDRQGAIKRLHSTLTRLGNASPSRP |
| | | | | | | PRQPFAPQRDIAIGVCFSRKNVLGVAVVDTRSQAVLEFC |
| | | | | | | NLRSLLTDDRLALLTKRAAKIPASRKGKRSVRQFQLKDY |
| | | | | | | RLFNRWRRLRHQNLTQRGDQQRHGLYAESSQESNLAQH |
| | | | | | | LNRVIAKKLLQLAQQWQASRLTLPDFGNLRESVECEMQ |
| | | | | | | ARARRKFPDDNVKLQKQYAKELRMFHHRWNHKQLAQC |
| | | | | | | LRACAARTGVPVITGTQPKEGELRDKAVALALAG* |
| MG64 | 292 | MG64-85 | protein | unknown | uncultivated | MSIITIHCRLVASEPIRRHLWHLMTESNTPLINDLLNQVSQ |
| effectors | | effector | | | organism | HPDFETWQRRGTVPEKTVKELCEPLKASYPGQPARFYAS |
| | | | | | | AILMVTYTYESWLALQQNRRRRLDGKQKWLHVVKSDA |
| | | | | | | ALLELSGTTLKAIQQQAQTILNQIDVGPETQGLPNAKRRK |
| | | | | | | PAQKQAKSASTASLMTRLFEAYEATDEILSRCAIAYVIKN |
| | | | | | | GCKIPETEEDPENFAHRLRRKQKEIEQLEAQLQARLPKGR |
| | | | | | | DLTGEEFLETLAIATQQISESVAQAREWQAKLLTRPASLP |
| | | | | | | YPIIYGSSTDVRWGNTAKRITVNFSGIDKYLKANDPDLAA |
| | | | | | | WFKTTKASPFQVYCDQRQLTFFQRFLDDWQTYQANKDT |
| | | | | | | YPAGLLTLSSAMLAWREGKGKGEPWHVNHLALYCSFDT |
| | | | | | | RLMTAEGTLEVQQEKAAKALKNLAHANPDPRNQSTLNR |
| | | | | | | LQHLPDRPSQKLYQGKPDILVGLSIGLANPVTAAVVNAS |
| | | | | | | KGNLLTYCTPRTLLGDHYHLLNRHRQHQQQNVLQRHKN |
| | | | | | | QQRGVAYQPSESELGQYVDCLLAKAIIQLAQAYKAGSIVI |
| | | | | | | PNLTHLRELLASEITARAEQKASLVEAQDKYAKEYRQTI |
| | | | | | | HRWSYNRLIEAIRSKAQQLGITVESGFQPLQGNPQEQAK |
| | | | | | | DVAIAAYHARAINAK* |
| MG64 | 293 | MG64-86 | protein | unknown | uncultivated | MSHITIQCRLVASLPTRRQLWELMADKNTPLINELLALV |
| effectors | | effector | | | organism | ANHPDFETWRQKGKLPSGTVKQLCQPLKTDPRFISQPAR |
| | | | | | | FYTSAIKVVDYIYKSWLALMKRLQYQLEGKTRWLEMLK |
| | | | | | | SDAELVESSGVTLETLRSKATEILAQLTPESDSVASQPPK |
| | | | | | | AKSKKKKKSKALDSKPNVSHILFDAYRNTADILNLCAISY |
| | | | | | | LLKNGCKINDKEEDQNKFSQRRRKVEIQIQRLTEKLTARI |
| | | | | | | PKGRDLTNTRWLETLAEATSCVPQNEAQAKYWQDNLLK |
| | | | | | | GFSLVPFPIIYETNEDMTWFKNVSSRLCVKFSGLGEHTFQ |
| | | | | | | VYCDQRHLHWFQRFLEDQEIKKNSKDQHSSGLFTLRSSS |
| | | | | | | MAWQEGEGKGEPWNLHHLTLYCCVDTRLWTAEGTKQV |
| | | | | | | KEEKATEIAKILTKAKEKGDLNQQQQSFIQRKNSTLTRIN |
| | | | | | | NPFPRPSQPLYQGQGNILVGVSLGLEKPATVAVVDAIAH |
| | | | | | | KVITYRSIRQLLGENYKLLNRQRQAQRSSSHERQNAQRR |
| | | | | | | DAFNQLGESELGEYIDRLLAKEIVAIAQKYQAGSIVLPKL |
| | | | | | | GDMREIVQSEIQALAEQKCPEFLEGQQKYAKQYRVSVH |
| | | | | | | QWSYARLIDCIQTQAKKLGIAIEEGQQPVRGSPQDRAKEL |
| | | | | | | AIAAYHLRSKA* |
| MG64 | 294 | MG64-87 | protein | unknown | uncultivated | MSMFTIQCRLCANEETRRSFWKWMEKYTLLMNELLENI |
| effectors | | effector | | | organism | AQNPQFPEWQKKDNISRVEVREILKPLKESSRYEGLPGRF |
| | | | | | | YTSAELISCGIYKSWLALNKRRKLQAIGKERWIKAVESEF |
| | | | | | | ELSATTEFNSDEIRSEAHRILEKETQELKKKERNPKELIAIL |
| | | | | | | LNRHEKTEHSLSRRAINHLLINNLQINEEELNLDKLSERL |
| | | | | | | DKKKVEVRRLKEQLISQLPKGRDSTRQQYLQILDHISVSV |
| | | | | | | LPELSDDPQKLEAELDKLTIQQQLPLFNELPYPIRFDSSRD |
| | | | | | | LYWSIQSQSPSNPSENGSHQQLPKNEKPQTKHDQRAKDR |
| | | | | | | ICVEFKGTKDYIFKIQCDRRQLPLFQQFLTDYQTYTQLPE |
| | | | | | | EERFSEALFALRSARLIWRKDDDSRHSSKKKRTTDKQED |
| | | | | | | QLKPWNTHRLYLHCTVDRRRLTAEGTEQVREEKKREVI |
| | | | | | | KKLKGRDRLEEAQLQELGLTKNQISDVKRKRSTLNRLKN |
| | | | | | | YSPPPRPRVPLYQGQPHIVVGVSFSRHQPVAIAVVDVAK |
| | | | | | | AEVLECQSAKELLNRGEAQYIWRNGQKEPLSKNGSERR |
| | | | | | | HPNGGKLLIRKRKRVRFKPYRLVEQLHRRHQQHPPRRAE |
| | | | | | | QQKQNCYTNNNSDSNLGLYVDRLIAAKIVELALKRKAG |
| | | | | | | TIAIPQLEGIRESVESDIRAQAERLFPNEKERQKEYAKHYR |
| | | | | | | ASFHRWSYARLSECIRECAKREGIAVVESKQSSQGDLEQ |
| | | | | | | KAIAIALSSYNVKTS* |
| MG64 | 295 | MG64-88 | protein | unknown | uncultivated | MVVRTIRCRLTASRETRQFFWEKMVAYTCLINQLFSKVA |
| effectors | | effector | | | organism | QDEQFNDWQQSSSVPRKPLEEIIKTIEKESGSYHLPARFYT |
| | | | | | | SAVLMTQYVYKSWFALQKRRQWQIQGKRRWLEIMKQD |
| | | | | | | SLLAHTDFSPETIYAKAREILSQTSNNRNEPKKKRSEGKK |
| | | | | | | SLLGSLMTKFEETDDLLTKCAVLHLLKNDFEVSEETHDD |
| | | | | | | PDDFKLRLESKRIEIERLEEQLQSRLPKGRDPTGDRFAENL |
| | | | | | | IEAIALPDDTVSNYSDLVFSSWLEQKQIKLLNPLPYPIIWG |
| | | | | | | SADDLRWTSEPRKLPPNAPRASSNTKKKKKPAKKKQITS |
| | | | | | | EDIIGVRFKGLSAHTFKVQCDRRQLPIFRQFFTDYKAYNA |
| | | | | | | LPEEERFSQKIFALCSAQLIWCQDSSKSKQKKPKDNPSKE |
| | | | | | | SWDSHRLYLHCTIDTQYLTAEGTADAIRLAKQKILKELG |
| | | | | | | ERATIPPEEIDSLDLTQPQKSHIKRKRTTLKRLDNPSPIRPR |
| | | | | | | REEYQGNPLITVGISLSRQMPLTACVVDIRTSKVLECQAT |
| | | | | | | KRLLLIKKFKIKSKKHNAHQLKRAHWRLVNKLNLRKKR |
| | | | | | | NSVQRQSKQKQDAYRESESESNLGAYTERLLANRVVAL |
| | | | | | | AMAWEAGSIVVPDLKNIREVAESDIKARAQERFPHEKQL |
| | | | | | | QKQYCKDLRASYHRWSYSRLVGYISDRAALFGISVMTG |
| | | | | | | KQPTHMSLPDSALHVAMSGHQLSAV* |
| MG64 | 296 | MG64-89 | protein | unknown | uncultivated | MSKLTIQCRLVACEDTRRQVWEMMAGRYAPLIATTLEQ |
| effectors | | effector | | | organism | VSQHKDFPQWVSAGEIPAQVVKNLVNQAQSGLPARWCA |
| | | | | | | SAQRQVQETYKAWLTKRRKLQQKLQGQQTWLSVLRPD |
| | | | | | | AELAQEAGLSLEEMKIRAQALLHREINNWFQVYQQCQD |
| | | | | | | VVERSIFAYLLKHRLTVPTEPEDTDKLRRKRRQVEIKIER |
| | | | | | | LETQLAGRSPQGRDLTGSRYAAALNEGEQCYWENDADF |
| | | | | | | LAWQAEILSRPDSLPPPVEYATNTDMTWHKDEQGRLAV |
| | | | | | | TFNGLGKLKFKIACDQRQLHWFQRFYQDQEQFKSQKGQ |
| | | | | | | RSQALFTLRSAELLWKPGNRSGDPWQANFLYLHCTVDS |
| | | | | | | RLWTQEGTAMVQQEKAKKSQAIVKKLSERSDLTAQQKD |
| | | | | | | CLQRHQSTLARLHMGYDRPQRRMYQGKSHLVVGISLDM |
| | | | | | | ENLVTVALVDVVKQKVITGCTMKSLLGQDYALVQRLRY |
| | | | | | | EKRQNSHLRKVAQERGSKIVNYEANLAIHVERLLVKAIIH |
| | | | | | | FAQQHLAGSLCVPTLKDIRETIQAHLQCRAEERFPDSKEL |
| | | | | | | QRRYAKEYRINAHRWSYNRLLKLLNQQAKFAGLVVEQG |
| | | | | | | VQSAGETALERALGVALSAYYQRSAA* |
| MG64 | 297 | MG64-90 | protein | unknown | uncultivated | MSQITIQCRLIAKESTRRQLWELMAHKNTPLINELLERIG |
| effectors | | effector | | | organism | QHPDFLTWRQNGKLPPGFIKQLGESLKTDPRYAGQPSRF |
| | | | | | | YMSAIALVNYIYKSWFALMKRLQYQLEGKTRWLEMLKS |
| | | | | | | DAELLETSGVTLEILRHQATEILTQLTPQSDSPKPKNTGK |
| | | | | | | KAKKPKASESDRTLSHSLFAAYRQSEDNLTRNAIAYLLK |
| | | | | | | NGCKLTDKEENPEKFAKRRRRVEIQIQRLTEQLTARIPKG |
| | | | | | | RDLTNAQWLETLAIASTTVPETDTQAKSWQDRLLKQFSV |
| | | | | | | VPFPVTYETNEDLTWFKNTSNRLCVKFSGLSEHSFQIYCD |
| | | | | | | QRQILWFERFLEDQQIKRASKNQHSSSLFTLRSGRIAWQE |
| | | | | | | GEGKGEPWNLHRLTLYCTVDTRLWTAEGTEQVKEEKAA |
| | | | | | | EIAKVLTKTKEKGNLNENQQAFIQRKSSTLTRINNPFPRPS |
| | | | | | | KPLYQGQSHIIVGVSLGLEKPATLAVVDAIASKPLTYRSV |
| | | | | | | KQLLGKNYPLLNRQRQEKQRNSQQRHSAQKQSAYNHFG |
| | | | | | | ESELGEYVDRLLAKEIVAIAQTYQASSIVLPKLGDMREIV |
| | | | | | | QSEIQALAEQKCPEYLEGQRNYAKQYRVSIHQWSYGRLI |
| | | | | | | ECIKSQAAKIGIAIEEGQQPVRGSPQEKAREMAIASYQSRS |
| | | | | | | QV* |
| MG64 | 298 | MG64-91 | protein | unknown | uncultivated | MTLKTIECRLYAPPETLRHLWELMAKKNTPLINELLHGIS |
| effectors | | effector | | | organism | EHPDFDKWFKQKKLPQKEIKSLCDRLKTEQAYQNQPGRF |
| | | | | | | YSSAIALTEYIYKSWFAIQKKLQQRIEGKQRWLNLLKSDS |
| | | | | | | ELETECGQSLEKIEIEAKNILDRFEKDSSLTKKKKQPKSQS |
| | | | | | | KSEKTLFNYLFDQYNETTDPLNRCVLAYLLKNNCQIPEQ |
| | | | | | | DEDLDRYQFRRQKKEIEIKRLQTQRQNRLPKDRDLSGQL |
| | | | | | | WLKILGTVNNCVPQDELEAASWQADLLRKSPVIPFPVSY |
| | | | | | | ETNTDLIWSKDGREHFQVRENGLGKQHQFEIRCDKRQLC |
| | | | | | | WFQRFFEDGEILRCDREQYSSALFTLRSARLLWREGKKD |
| | | | | | | KDNPWEIHTLYLQCSVDTRFWTAEGTKQIASDKSATVQE |
| | | | | | | ILNNLKEKAELTPSQLAYQKRQQSTFTRITNPFPRPQKPL |
| | | | | | | YQGDPSIIMGVSLGLEKPATIAIVNVTNNRVLAYRSIKQL |
| | | | | | | LGKNYKLLNHQQRQKQKLSHLRHQAQKTESNNQFGESE |
| | | | | | | LGEYIDRLIAKAIVEIAQKYRVSSIVLPHLKQIREITDSELM |
| | | | | | | AKAQRKIPGYKEGQKKYIKQYRCNIHQWSYGRLIESIEQ |
| | | | | | | AAAKIGIDIEQIQQSRQGTPQEQAKQLAISAYNSRLQRAI* |
| MG64 | 299 | MG64-92 | protein | unknown | uncultivated | MGIITIHCHLGTIEPIRRLLWQAMVESNTPLISTLLRQVAN |
| effectors | | effector | | | organism | HSDFDTWQIKGSVPVKAVRTIGDPLKAHYPPQPGRFYAS |
| | | | | | | AYQMVSYTYESWLALQKKTKFSLEGKRRWLSIVKSDAE |
| | | | | | | LLELTGLSLESLRQSAREVLSQISAEIAAERVPDTQKNKP |
| | | | | | | KVKSRKSKKKSTGKDKDLIGKLFKVYGTTDDLTQRCILA |
| | | | | | | YLIKNGGTILDQEETTEAFARRVHRKQKEIARLENRLEAR |
| | | | | | | LPKGRDLTGDIFTDTLLLAQQQEPEDIAQMRDWQAKLL |
| | | | | | | MRPADLPYPIRYDSSTDMMWKPDDQGRISVNFNGLDKF |
| | | | | | | LKNSDPEVRSWLKEHQGYPFRIQCDQRQLPCFQRFLADW |
| | | | | | | QAYTADAENYPAGLLTLSSAMLAWRKGKKNGKGEPWN |
| | | | | | | IHQLALYCSFDTRLLTAEGTVEVQQEKIKKAQKQAKSAD |
| | | | | | | GKKLDEKQLQARTSNATTLRKLDNLPNRPGCKPYQARP |
| | | | | | | ELLLGISIGLSEPVTVAIVDAATHQVLTYRTSRTLLGEQH |
| | | | | | | RLLRRQRQKQQQNRLKRQQNQKQGIRHQPSESELGQYV |
| | | | | | | DCLLAKAITQLALTNQVSSIVLPDLLNRRDILDSEIQAKAE |
| | | | | | | RKCPGSISAQEKYAKDFRRSLHSWDYRRLIEAIRSSACKH |
| | | | | | | GIPLEETFLTASSDPKEQAKEIAIAAYQARTED* |
| MG64 | 300 | MG64-93 | protein | unknown | uncultivated | MTKASTIKIYEAKLIPSAWKRPTKKEPTSIATPSEEVKKQ |
| effectors | | effector | | | organism | VLLLSIRSTQLCKAIGDSLSKRDELDEWISKGAIPEKVLKA |
| | | | | | | EWEIARNQKCFRDMPSRFQTTALLRIQETFSGWFEQRKQ |
| | | | | | | KRREIDRSQRWLDIVKSDAELMEISGLDIEDIKAKALEILQ |
| | | | | | | EAKKISETSQAQPEPAPESCVSEEKNSADTTPTANPYPNQ |
| | | | | | | PYKSVFSILFEMYEEVVQHDLVATCAISHLLKNRASIATE |
| | | | | | | TEDTKAFEARIRKKKKEVERLKSQLNSRLPKFRIFSDQLL |
| | | | | | | PYLFDECADLDKAKPPSKAIGKNIPRKHSDLPYSVLFYSR |
| | | | | | | DDITWELIQRMNPNSQVLEDRIFIKIKGLDKYVKQQGIAQ |
| | | | | | | PAVFEICCDVRQLSYFKRCHEEWQLYSKNRNDYSTRDFL |
| | | | | | | LQSASLVWKKKNVPVGQRKSSNELDRYEPYLQITIDTDK |
| | | | | | | LTFESSEKKRLVELEAVERIIASYEAKQQDGELTIQQQKG |
| | | | | | | LQRSLTTRRKLESNSFPRPSKPLYQGNQNLVLGVSFGLEK |
| | | | | | | PVAVAIVDLTKEQTITVRSAKQFLGANHNQLSAYRHEQR |
| | | | | | | HNSSQRRKNQRQRKSAEISEHRRGMHIDRVMAKAIVNL |
| | | | | | | AQEYKAGLIVLADCKGIRDRIQSGIEAKAEHKYSKDIERQ |
| | | | | | | KAYLKQYRVNVHKWDFRRLSQCIRGKAGKEGIPVEIVK |
| | | | | | | QRYQGDLPAKAAQVAFDGAKVLSS* |
| MG64 | 301 | MG64-94 | protein | unknown | uncultivated | MLVDRLKLDPRFGEQPVWYYVSAQKQVAYTFRSWLSSQ |
| effectors | | effector | | | organism | RRKQWRLEGKRRWLEILRPDAELAEIAKCSTEALRSAAS |
| | | | | | | RILKEVDDPAPFNFLLKEYGTVNSRKRQCALAYLLKRNA |
| | | | | | | KLDPEVEDLEKLEARRGKTEIQIKRLEMQLQANLPKGRD |
| | | | | | | LTGQIQSEALAQCVQTAFIDDAVYSAWQSTITRKPASLPF |
| | | | | | | PIIYETVESLVWSKDCYGRYSVCFQGQGTSTHTFKIYCDK |
| | | | | | | PHQHWFERFWIDQETKRSGGDQHSAGLFTLRSARLSWV |
| | | | | | | PSKDHQDEPEPWNRYYLNLSCTVDTDLWTQEGTQLVLQ |
| | | | | | | KKAASTASKLQAMREKESLNENQQGYVRRLESTMKRLQ |
| | | | | | | TPYPRPSRDLYQGRSDILVGVSMGLDKPATVAVINVLNG |
| | | | | | | DVLAYRSTKQLLGEKYPLLQRARSERAKIAHQGHRQRR |
| | | | | | | KGEKNVAQESNLGEYVDRLLAKAIVEVAQQYWAGGIVL |
| | | | | | | PDLSHIREIIEAEVKQKAAAKVPDFVDGQKQYAKAYRAQ |
| | | | | | | VHQWSYNRLQNFITSKAEQSGLSVETTKQEYSGSPQEKA |
| | | | | | | KLLCFAGYENRLVLLS* |
| MG64 | 302 | MG64-95 | protein | unknown | uncultivated | MSQITIQCNLVASEATRQYLWHLMADIYTPFINEMLATIA |
| effectors | | effector | | | organism | QHPNFEEWSQSGKIPADVFEDIRKTLKAHPDFQGMPGRW |
| | | | | | | YYAGRDLVKRIFKSWLALRRRLRHQLSGQTHWLEIFQSD |
| | | | | | | DDLVAACGQDLPAIRAEAASILTKIQIEAPNTSKQPKKTK |
| | | | | | | QPKKAGSKTQKPEEEQRNRNLFPALFKEYDGAETELVKC |
| | | | | | | AIACLLKNNCQIPTKAEHPEKFQKRRRKTEIRVERIIEQLA |
| | | | | | | RTRLPKGRDLTNEKWLDTLKMAVQQVPKDETEAAAWE |
| | | | | | | ADLQTDSSPLPFPIAYESNEDLKWSQNAKGRLCVRENGL |
| | | | | | | GKHTFEIYCDTRQLHWFKRFLDDQTIKKQGGNSHSAGAL |
| | | | | | | TLRSGRISWRLDSSKGNPWDRNRLVLFCSVDTLLWTKEG |
| | | | | | | TEKASQEKASKIAQVISGTKAKGNLTSKQEDFVRKREKT |
| | | | | | | LALLQNPFPRPSRPLYQGSPAILAGVSFGLDKPATLAIVD |
| | | | | | | VTTGKAIAYRSIRQLLGDDHKLLNRQRQRQRQKAQRRRS |
| | | | | | | NQLKFASNRISEGGLGGQIDSLIAKAIVQIAQQYNASSIVL |
| | | | | | | GDLANIREIIESEIQAKAEQKTTLKEIQAKYARDYRASIHR |
| | | | | | | WSYKRLAQKIESNALQAGLIVATIKQPLAGSPQDKARDV |
| | | | | | | AIAGFQSRSVSKILDTGS |
| MG64 | 303 | MG64-96 | protein | unknown | uncultivated | MQSEIDLLKTTEFELSEITAAAKLALNSARKQKKKSDKSV |
| effectors | | effector | | | organism | EGSSPSLFSILIDIQFKTKSPLKKRGINHLLLNDLNIRQREF |
| | | | | | | ELSDLETRLEAKFLEVEDLENRLRSRLPKGRDPDGQRYV |
| | | | | | | SALAEAASISEQYLSPERINEIQASIPIYNELPYPLIYEGGSN |
| | | | | | | ISWILLESANSKSKSGRLQVYFSGISELKFSIQCSRRQLPIF |
| | | | | | | RGFYEDKTENKNRFRREEIPFSEGLNRFRSAQIIWKPDSNF |
| | | | | | | QFRKKRGEITSFPWEVNRLHFHCSVNRVTLSAEGTEQLR |
| | | | | | | QAKLKKLTTKDEKSLIPRKRTELERLRNAEPPPRPSVPIHI |
| | | | | | | RDPNIVIGVCFSPDEPVIVVPIDLEIEAALYALNTKALLNQ |
| | | | | | | EKKTIWRNGKKETVSDSGELQLHSNGGKLSSRKPGQWF |
| | | | | | | VQKPYDLVTRLNTLTEQEAKLRKREQSKGKYENSESLSN |
| | | | | | | LSLYVCRLIAARLVELSLKLNVSRVILPDLEGIRDWVQAII |
| | | | | | | SAKAAKAFPDSKKQQKQFLQQFRVKYHRWNYRKLCQEI |
| | | | | | | ESCARKSGLQVTYARQPNLHEISEQVSAFSRDEEFYTFKV |
| | | | | | | AAEKMALPQEL* |
| MG64 | 304 | MG64-97 | protein | unknown | uncultivated | MHSILLNIYGIIEHNVFLNLKSMSQNTIQGRLVASVVTRQ |
| effectors | | effector | | | organism | QLWKLMADKNTPLINELVLQVAQDPDFETWREKGKIPT |
| | | | | | | GIVKQLCAVLKTDSRFIGQPGRFYTSAINRVNYIYKSLLA |
| | | | | | | LMKKLQYQLDGKNRWLEMLKSDIELVEASGVNLESLRL |
| | | | | | | KAAEILAQVTPQSDMVEPQPAKGKKRQKTKKSKDSDSD |
| | | | | | | CAERTLRDRTVSKSLFEAYSNTEDNLTRCAISYLLKNRCK |
| | | | | | | VSEKEEDLKKFAQRRRKVEIQIERLTEQLTARIPKGRDLT |
| | | | | | | DTKWLETLIIATQKVPSNEAEAKSWQDNLLKKPSRVPFP |
| | | | | | | VAFETNEDMTWFKNKDGRICVKFNGFSEHTFQIYCDSRQ |
| | | | | | | LHWFQRFYEDQQIKRIGKKQHSSSLFTLRSGLIAWQEEEG |
| | | | | | | KGDPWDVNHLILYCSVDTRLWTDEGTNLVREEKAEEIA |
| | | | | | | KILTKIKAKDDLNDKQLAYIKRKNSTLARINNPFPRPNKP |
| | | | | | | LYKGRSHILVGVSFGLEKPTTVAVVDGSTGEVLIHRSIKQ |
| | | | | | | ILGDNYRLLNRQRQQKHSLSHQRQIAQTLAAPNQLGESE |
| | | | | | | LGQYVDRLIAKEIIAISQTYKAGSIVLPKLGDMREQVQSE |
| | | | | | | VQAKAEQKSDLIKVQKKYAKQYRVSVHQWSYSRLITSIQ |
| | | | | | | NQAKKAGIVVEEAKQSVRGSPQEKAKELAIAAYYSRKIN |
| | | | | | | * |
| MG64 | 305 | MG64-57-B | protein | unknown | uncultivated | MSQDSQNSLVMDTDDEHPQIGSKGKLINSHKLPSNELLT |
| transposition | | transposition | | | organism | DEVNLRMEVIQSLTEPCDRKTYAIRKKEAAEKLGVSIRQ |
| proteins | | protein | | | | VERLLKKWREERLVGLATTRSDKGKYRLDQEWIDFIIDT |
| | | | | | | FKQGNEGSKRMTRHQVFLRVKGRAKQLNLNKGEYPSHQ |
| | | | | | | SIYRILDEYIEQKQRKLKARSPGMLGERLTHMTRDGRELE |
| | | | | | | VECSNDVWQCDHTRLDVRLVDEYGVLDRPWLTIVIDSY |
| | | | | | | SRCLVGFYLGFDHPSSQIDALALRHAILPKSYSSEYQLRN |
| | | | | | | EWRTYGKPNYFYTDGGKDFTSIHTTQQVAVQIGENCALR |
| | | | | | | RRPSDGGIVERFFKTLNEQVLNLLPGYTGSNVQKRPENV |
| | | | | | | DKDACLTLKDLEKVIVRYIVGEYNQHTDARMKDQSRIG |
| | | | | | | RWEAGLMADPYLYDELDLAICLMKRERRKVQKYGCIQF |
| | | | | | | ENLTYRAEHLRGRDGEIVAFRYDPVDVTTLFVYKINADG |
| | | | | | | TEEFLDYGHAQGFETERLSLRELKAINKRKKEASQEINND |
| | | | | | | SILEAMLDRQAFVEQIVKQNRKQRREAANEQVNPVESVA |
| | | | | | | KKFTVPEPKEIAVESEPEAELPKYEVRYMDEFYEED |
| MG64 | 306 | MG64-57-C | protein | unknown | uncultivated | MTTNAISLAKQFGVIEEPTPEVQAEIERLSREPYLELDQV |
| transposition | | transposition | | | organism | KYCHAWMYELVISRMTGLLVGDSRCGKTVTCKAFAHR |
| proteins | | protein | | | | YNKSRQAKGQRLKPVVYIQIPKNCGSRDFFIKILKALNKP |
| | | | | | | SNGTISDLRERTLDSLAIHQVEMLIIDEANHLKFETFSDVR |
| | | | | | | HIYDDDNLRISVLLVGTTSRLLAIVKRDEQVINRFLEQFEL |
| | | | | | | DRLEDAQFKQMIQIWERDVLRLPEESKLASGDNLKLLKQ |
| | | | | | | ATKKLIGRLDMILRKAAIRSLLRGHKKVDKDVLKEVIAA |
| | | | | | | SKL |
| MG64 | 307 | MG64-57-Q | protein | unknown | uncultivated | MEENVDEKRQLWLTRVEPYEGESISHFLGRFRRAKGNKF |
| transposition | | transposition | | | organism | SAPSGLGDVAGLGAKLARWEKFYFNPFPARQELEALAA |
| proteins | | protein | | | | VVEVDADRLREMLPPPSVGMKHSPIRLCGDCYAESPCHK |
| | | | | | | IEWQFKVTVGCYRHKLRLLSKCPVCGKPFPIPALWVEGH |
| | | | | | | CPRCFTPFAQMAKSQKYY |
| MG64 | 308 | MG64-58-B1 | protein | unknown | uncultivated | MKDAESTTNSPMTHASIVDAENGKAEANIIVSELSDEALL |
| transposition | | transposition | | | organism | KMEVIQSLLKNSDRSTYGELLKQSAEKLGRSVRTIRRLV |
| proteins | | protein | | | | DKWEKEGLAGLVQNQRDDKGKHRVDKYWQEFVLTTY |
| | | | | | | KENNKGSKRMTRQQVFIRAKARADELGIEPPSHMTVYRI |
| | | | | | | LKPIIDKQEQAKSIRSPGWRGSRLSVKTRDGKDLQVEHSN |
| | | | | | | QVWQCDHTRVDVLLVDQHGKILSRPWLTTSIAIRVALW |
| | | | | | | VLI |
| MG64 | 309 | MG64-58-B2 | protein | unknown | uncultivated | VGINLGYDAPSSTVVALALRHAILPKQYSSEYGLHEEWG |
| transposition | | transposition | | | organism | TSGLPQNFYTDGGKDFRSNHLQQIGVQLGFVCHLRDRPS |
| proteins | | protein | | | | EGGSVERPFKTLNTELFSTLAGYTGSNVQERPEEAEKEAS |
| | | | | | | FTLRQLEKMLVRYIVDNYNQRIDARMGDQTRFQRWESG |
| | | | | | | LIAMPDLLSERDLDICLMKQTRRQVQRGGYLQFENLMY |
| | | | | | | RGELLAGYAGESVVLRYDPKDITTILVYRIEGDKEIFLAR |
| | | | | | | AYAQDLETEELSLDEAKASSRKVREAGKAISNRSILAEIR |
| | | | | | | ERETFPTQKKTRKERQKLEQAEVKKAKQLTPAETEEEIIV |
| | | | | | | VSIDAKPTAKNPLESELCTESGEPDMPEVLDYEQMREDY |
| | | | | | | GW |
| MG64 | 310 | MG64-58-C | protein | unknown | uncultivated | MVAKEAQEVAKQLGDIPVNDEKLQAEIHRLNRKGFVPL |
| transposition | | transposition | | | organism | EQVQTLHDWLEGKRQSRQSGRVVGESRTGKTMGCDAY |
| proteins | | protein | | | | RLRNKPKQEAGKPPTVPVAYIQIPQECGAKEFFGVILEHL |
| | | | | | | KYQVTKGTVAEVRDRALRVLKGCGVEMLIIDEADRFKP |
| | | | | | | KTFAEVRDIFDKLEIPVILIGTDRLDAVIKRDEQVYNRFRS |
| | | | | | | CHRFGKLSGEEFKRTVEIWEKKVLQLPVASNLSSKTMLK |
| | | | | | | TLGEATGGYIGLMDMILRESAIRALKKGLQKIDLNTLKE |
| | | | | | | VTAEYR |
| MG64 | 311 | MG64-58-Q | protein | unknown | uncultivated | MESEYIKAWLFQVEPFEGESLSHFLGRFRRTNDLTPGGLG |
| transposition | | transposition | | | organism | SQAGLGGAIARWEKFRFNPPPSLGQLEKLAVVAGIDAGR |
| proteins | | protein | | | | LVQMLAPPGVSMKLEPIRLCAACYAESPCHKIEWQFKET |
| | | | | | | QGCKHHKLRLLSECPNCGARFRVPALWVDAWCHRCFTL |
| | | | | | | FGGMVNHQKPC |
| MG64 | 312 | MG64-59-B | protein | unknown | uncultivated | MQDAEFSTASTTKASSTDVSSTEASIIVSELSDEALLKME |
| transposition | | transposition | | | organism | VIQSLLENSDRTTYTKCLQEAAKKLGKSVRTVRRLVDK |
| proteins | | protein | | | | WEQEGLAGLVQNQRVDKGKHRVDENWQEFVLKTYKEG |
| | | | | | | NKGSKRMTRQQVFIRAKARADELGVKPPSHMTVYRILQP |
| | | | | | | LIDKIEQAKSIRSPGWRGSRLSVKTRDGKNLQVEHSNQV |
| | | | | | | WQCDHTPADILLVDQHGKLLSRPWLTTVVDSYSRCIMGI |
| | | | | | | HLGYDAPSSQVVALALRHAILPKQYNSEYGLHEEWGTY |
| | | | | | | GLPQHLYTDGGKDFRSNHLQQIGVQLGFVCHLRDRPSEG |
| | | | | | | GSVERPFKTLNTELFSTLQGYTGSNVQERPEEAEKEACLT |
| | | | | | | LRQLEQMLVRYIVDNYNQRLDARMGDQTRFQRWESGLI |
| | | | | | | AAPDLLSERDLDICLMKQIRRQIQRGGYLQFENLMYRGE |
| | | | | | | LLAGYAGESVVLRYDPRDITTILVYRTEGDKEVFIARAIA |
| | | | | | | QDLETEELSLDEAKAISRRVREAGKAVSNRSILAEVRERE |
| | | | | | | VFPTQKKTKKERQKLEQAEVKKAKQLIPVEPEESVEVVSI |
| | | | | | | DSETEPDMPEVFDYEQMREDYGW |
| MG64 | 313 | MG64-59-C | protein | unknown | uncultivated | MSAKEAQAIAQQLGDIPVNSEKVQAEIRRLNRKGFVPLQ |
| transposition | | transposition | | | organism | QVQTLHDWLEGKRQSRQSGRVVGESRTGKTMGCDAYR |
| proteins | | protein | | | | LRNKPKQEAGKPPTVPVAYIQIPQECGAKEFFGVIMEHLK |
| | | | | | | YQVTKGTVAEIRDRTLRVLKGCGVEMLIIDEADRFKAKT |
| | | | | | | FAEVRDIFDKLEISVILVGTDRLDAVIKRDEQVYNRFRSC |
| | | | | | | HRFGKMSGQDFKQTVEIWEKQILKLPVASNLGSKTMLKT |
| | | | | | | LGEATGGYIGLMDMILREAAIRALKKGLQKIDLETLKEV |
| | | | | | | AAEYR |
| MG64 | 314 | MG64-59-Q | protein | unknown | uncultivated | MMEAENIKPWLFQVEPLDGESLSHFLGRFRRANDLTPSG |
| transposition | | transposition | | | organism | LAKTAGLGGVVARWEKFRFNPPPSRQQLLSLAVVVGIDA |
| proteins | | protein | | | | DRLTLMLPPVGMGMKMEPIRLCGACYGESPCHKIEWQF |
| | | | | | | KVMQGCKLHNLSLLSECPNCGARFKVPALWVDGWCQR |
| | | | | | | CFTPFGKMIEGQYKVLHSSI |
| MG64 | 315 | MG64-61-B | protein | unknown | uncultivated | MSGFHSMADEEFEFTEGTTQVADAILLDKSNFVVDPSHII |
| transposition | | transposition | | | organism | LATSDRHKLTFNLIQWLAESPNRAIKSQRKQAVANTLDV |
| proteins | | protein | | | | STRQVERLLKQYDEDKLRETAGIERADKGKYRVSEYWQ |
| | | | | | | NFIKTIYEKSLKDKHPISPASIVREVKRHAIVDLKLKLGEY |
| | | | | | | PHQATVYRILDPLIEQHKRKTRVRNPGSGSWMTVVTRDG |
| | | | | | | ELLKAEFSNQIIQCDHTKLDVRIVDSDRNLLSDRPWLTTI |
| | | | | | | VDTYSSCIVGFRLWIKQPGSTEVALALRHAILPKKYPDDY |
| | | | | | | QLNKSWDICGHPYQYFFTDGGKDFRSKHLKAIGKKLGFQ |
| | | | | | | CELRDRPPEGGIVERIFKTINTQVLKELPGYTGANVQERP |
| | | | | | | ENAEKEACLTIQDLDKILASFFCDIYNHEPYPKEPRDTRFE |
| | | | | | | RWFKGMGEKLPEPLDERELDICLMKEAQRVVQAHGSIQF |
| | | | | | | ENLIYRGEFLKAHKGEYVTLRYDPDHILSLYIYSGETDDN |
| | | | | | | AGEFLGYAHAVNMDTHDLSIEELKALNKERSNARKEHF |
| | | | | | | NYDALLALGKRKELVEERKEDKKAKRNSEQKRLRSASK |
| | | | | | | KDSNVIELRTTRASKSLKKQENQEVLPERISREEIKFEKIE |
| | | | | | | QQPQENLSTLPNPQEEERHKLVISSRKKNLQNIW |
| MG64 | 316 | MG64-61-C | protein | unknown | uncultivated | MAQPQLATQPIVEVLAPKLDIKAQIAKTIDIEEIFRTYFITT |
| transposition | | transposition | | | organism | DRASECFRWLDELRILKQCGRIIGPRNVGKSRAALHYRD |
| proteins | | protein | | | | DDKKRVSYVKAWSASSSKRLFSQILKDINHAAPTGKRQD |
| | | | | | | LRPRLAGSLELFGLELVIIDNAENLQKEALIDLKQLFEECN |
| | | | | | | VPIVLAGGKELDELLQDCDLLTNFPTLYEFERLEYDDFKK |
| | | | | | | TLTTIEFDVLSLPEASNLTEGNMFEILAASTQARMGILIKIS |
| | | | | | | CYAA |
| MG64 | 317 | MG64-61-Q | protein | unknown | uncultivated | MVQNIFLSKTEIEINEDDEIRPRLGYVEPYEGESISHYLGR |
| transposition | | transposition | | | organism | FRRFKANSLPSGYSLGKIAGIGAVTTRWEKLYFNPFPTRQ |
| proteins | | protein | | | | ELEDLASVVGVNVDRLMEMLPSQGMTMKPRPIRLCAAC |
| | | | | | | YAEVPCHRMEWQFKDKIRCDRHNLRLLTKCTNCETPFPI |
| | | | | | | PADWVKGECPHCSLPFAKMVKKQRRD |
| MG64 | 318 | MG64-60-B | protein | unknown | uncultivated | MNERQDESLEAASEISDEIVLSDRAFDTDPSKILIGASDPQ |
| transposition | | transposition | | | organism | KLRFRLIEWLSESPNRKVKAERKQAIIKTLDVSTRQVERL |
| proteins | | protein | | | | LNQYYEDKLRETAGTERSDKGDHRIDDYWQDYIREVYE |
| | | | | | | KSLKDKHPLKPADVVREVQRHAVIDLRHEEGDWPHPAT |
| | | | | | | VYRILKPLVERHKRKQKIRNPGSGSWMAVETRDGKLLK |
| | | | | | | ADYSNQIVQCDHTKLDIRIIDKEGKLLNWRPWLTTIVDTF |
| | | | | | | SSCLIGYHLWHKQPGSHEVALTLRHAILPKQYPAEYELK |
| | | | | | | KPWDIYGAPLQYFFTDGGKDLSKSKLIKAIGKKLGFQCE |
| | | | | | | LRDRPIQGGIVERLFKTINTEVLAALPGYISKEEGGAERAE |
| | | | | | | KEACFTLEDIDKILAGYFCDDYNHKPYPKDPRDTRFERW |
| | | | | | | FRGMGNKLPEPLNERDLDICLMKEEQRFVQAYGSVYFEN |
| | | | | | | LTYRCEELRSLKGEPVTLTYDPDHVLTLYIYRQSTGDEVR |
| | | | | | | EFIGYAHAINMDNQDWSLDELKQFNKVRNTAKRDHSNY |
| | | | | | | DALLALNQREKLAEQRKQEKKERQRSEQKKLRGKSKQN |
| | | | | | | SNVVELRKERAGKSAGSAEPIELLPERALPEMSEPQTPAP |
| | | | | | | PSQTPEPAASKPPTEERHRLIIPKNQTLKRIW |
| MG64 | 319 | MG64-60-C | protein | unknown | uncultivated | MAQSQLATQVPVEVLAPQLNLTDVLAKMVAIEELFETAF |
| transposition | | transposition | | | organism | IPTDRASQCFRWLDELRLLKQCGRVIGVSEVGKSRTAKH |
| proteins | | protein | | | | YREDNPKRVAYVKAWTNSSSKRLFTQILKEIKHAAPNGR |
| | | | | | | HKDLRSRLAGSLEPFGIELVIVDNANNLQREALLDLKQLF |
| | | | | | | EESKVSIALVGGHELDTMLDDFDLLTCFPNLFEFARLDCE |
| | | | | | | DFEKTLKTIELDILALPEASNLTEGKIFEILASATEARMGM |
| | | | | | | LIKILTKAILRSLNKGSGKVDAGALEAIANRYGKKYKPPT |
| | | | | | | AKK |
| MG64 | 320 | MG64-60-Q | protein | unknown | uncultivated | MFEADDDQLRLGCVEAYDGESISHYLGRLRRIKANSLPS |
| transposition | | transposition | | | organism | AYSLGQLVGLGGTLARWENLRFNPFPSDAELETLGKLM |
| proteins | | protein | | | | QVAPVKLAQMLPLKGVTHQPAPIKLCGACYAETPCHRIG |
| | | | | | | WQAKSKQETLGCNRHQLRLLSKCPQCKRPFPIPSLWKEG |
| | | | | | | SCNNCSLPFAKMLRFQKAL |
| MG64 | 321 | MG64-65-B | protein | unknown | uncultivated | MGETLNSDEGNTSLAFDSSDELDEILEDEDAEPTNTIITQL |
| transposition | | transposition | | | organism | SDEAKLRMEVLQSLIEPCDRKTYGIKLKQAAEKLGKTVR |
| proteins | | protein | | | | TVQRLVKKYQEQGLSAITEPERSDKGNYRIDEEWQEFIV |
| | | | | | | KTYKEGNKSGRKMTPAQVAIRVQVRAEQLGLERYPSHM |
| | | | | | | TVYRVLNPIIERKEQKQKVRNIGWRGSRVSHQTRDGQTL |
| | | | | | | EPRYSNHTWQCDHTKLDVMLVDQYGETLARPWLTKITD |
| | | | | | | SYSRCIMGIHLGFDAPSSLVVALAMRHAVLPKKYPSEYK |
| | | | | | | LHCEWGTFGIPENLFTDGGKDFRSEHLKQIGFQLGFECHL |
| | | | | | | RDRPSEGGIEERSFGTINTDFLSGFYGYLGSNIQQRPENAE |
| | | | | | | EEACITLRDLQLLIVRYVVDNYNQRLDARSGQTRFQRWE |
| | | | | | | AGLPALPSLIDERQLDICLMKKTRRSIYKGGYVSFENITY |
| | | | | | | RGEYLAAYAGEGVILRFDPRDISTVFVYRQDSGKEVLLS |
| | | | | | | QAHAIDLETEQISLEEAKAASRKIRDTGKQVSNKSILAEV |
| | | | | | | RDRDVFIKQKKKSQREHKKEEQAQVHSVYKPPQIHEPVE |
| | | | | | | QTQETPQLQKRRPRVYDYEQLRRDYDD |
| MG64 | 322 | MG64-65-C | protein | unknown | uncultivated | MTDESLRRWVQNLWGDDPIPEELMPQIERLITPSVVELD |
| transposition | | transposition | | | organism | HIQRIHDWLDSLRLSKQCGRIVAPPRAGKSVTCDVYKLL |
| proteins | | protein | | | | NKPQKRTGKRDIVPVLYMQVPGDCSAGELLTLILESLKY |
| | | | | | | DATSGKLTDLRRRVLRLIKESKVEMLIIDEANFLKLNTFS |
| | | | | | | EIARIYDLLKIAIVLVGTDGLDNLIKKEPYIHDRFIECYRL |
| | | | | | | QLVSEKKFAELLQIWEDEVLCLPVPSNLTRKETLMPLYQ |
| | | | | | | KTGGKIGLVDRVLRRTAILSLRKGLSNIDKATLDEVLEWF |
| | | | | | | E |
| MG64 | 323 | MG64-65-Q | protein | unknown | uncultivated | MENIEKKTRFFDIELLQGESLSHFLGRFRRENYLTTTQLG |
| transposition | | transposition | | | organism | KLTGLGAVIGRWEKFYFNPFPTQQELEALAGIVGVEVEK |
| proteins | | protein | | | | LREILPPKGVTMKPRPIRLCGACYAEVPYHRMKWQFKD |
| | | | | | | KMKCDRHNLGLLTKCTNCETPFPIPSDWVQGECSHCFLP |
| | | | | | | FATMAKRQKRY |
| MG64 | 324 | MG64-66-B | protein | unknown | uncultivated | MQLGFECHLRDRPSEGGIEERSFGTINTDFLAGLYGYLGS |
| transposition | | transposition | | | organism | NIQQRPENAEEEACITLRELHLLLVRYIVDNYNQRIDARS |
| proteins | | protein | | | | GNQTRFQRWEAGLPALPNLIEERQLDICLMKKTRRSIYK |
| | | | | | | GGYVSFENIMYRGDYLAAYAGESVLLRYDPRDISTVFVY |
| | | | | | | RQDSGKEVLLSQAHAIDLETEQISLEETKAANRKIRNTGK |
| | | | | | | QLSNKSILAEVQDRDTFIKQKKKSHKERKKEEQAQVRSV |
| | | | | | | KPSQTNKPVETVEEIPQPQKRRPRVFDYEQLRKDYDD |
| MG64 | 325 | MG64-66-C1 | protein | unknown | uncultivated | MAEDYLRKWVQNFWGDDPIPEELLPIIERLITPSVVELEHI |
| transposition | | transposition | | | organism | QKIHDWLDSLRLSKQCGRIVAPPRAGKSVTCDVYKLLNK |
| proteins | | protein | | | | PQKRTGKRDLVPVLYMQVPGDCSAGELLTLILESLKYDA |
| | | | | | | TSGKLTDLRRRVLRLLKESKVEMLVIDEANFLKLNTFSEI |
| | | | | | | ARIYDLLNVSIVLVGTDGLDNLIKKEPYIHDRFIECYRLPL |
| | | | | | | VSEKKFPEFVQIWEDEVLCLPVPSNLTKS |
| MG64 | 326 | MG64-66-C2 | protein | unknown | uncultivated | MPLYQKTSGKIGLVDRVLRRAAILSLRKGLKNIDKATLD |
| transposition | | transposition | | | organism | EVLEWFE |
| proteins | | protein | | | | |
| MG64 | 327 | MG64-66-Q | protein | unknown | uncultivated | MEIPAKEPRFFQVEPLEGESLSHFLGRFRRENYLTATQLG |
| transposition | | transposition | | | organism | KLTGIGAVISRWEKFYLNPFPTPQELEALAAVQNEFGNSR |
| proteins | | protein | | | | H |
| MG64 | 328 | MG64-68-B | protein | unknown | uncultivated | MGETLNSNEVDESLVLYDGSDEVDEISESEDTKQSNVIVT |
| transposition | | transposition | | | organism | ELSEEAKLRMEVLQSLIEPCDRKTYGIKLKQAAEKLGKT |
| proteins | | protein | | | | VRTVQRLVKKYQEQGLSGVSEVERSDKGGYRIDDDWQD |
| | | | | | | FIVKTYKEGNKGGRKMTPAQVAIRVQVRAGQLGLEKYP |
| | | | | | | CHMTVYRVLNPIIERKEQKQKVRNIGWRGSRVSHQTRD |
| | | | | | | GQTLDVHHSNHVWQCDHTKLDVMLVDQYGETLARPWL |
| | | | | | | TKITDSYSRCIMGIHLGFDAPSSLVVALAMRHAMLRKQY |
| | | | | | | SSEYKLHCEWGTYGVPENLFTDGGKDFRSEHLKQIGFQL |
| | | | | | | GFECHLRDRPPEGGIEERGFGTINTDFLSGFYGYLGSNVQ |
| | | | | | | ERPEGAEEEACITLRELHLLILRYIVDNYNQRIDARSGNQ |
| | | | | | | TRFQRWEAGLPALPNLVNERELDICLMKKTRRSIYKGGY |
| | | | | | | VSFENIMYRGDYLSAYAGESVLLRYDPRDISTVFVYRQD |
| | | | | | | SGKEVLLSQAHAIDLETEQISLEETKAASRKIRNAGKQLS |
| | | | | | | NKSILAEVQDRDTFIKQKKKSHKQRKKEEQAQVHSVKPP |
| | | | | | | QTNEPVETVEEIPQPQKRRPRVFDYEQLRKDYDD |
| MG64 | 329 | MG64-68-C | protein | unknown | uncultivated | MAEDYLRKWVQNLWGDDPIPEELLPIERLITPSVVELEHI |
| transposition | | transposition | | | organism | QKIHDWLDSLRLSKQCGRIVAPPRAGKSVTCDVYKLLNK |
| proteins | | protein | | | | PQKRTGKRDIVPVLYMQVPGDCSAGELLTLILESLKYDAI |
| | | | | | | SGKLTDLRRRVLRLLKESKVEMLVIDEANFLKLNTFSEIA |
| | | | | | | RIYDLLKISIVLVGTDGLDNLIKKEPYIHDRFIECYRLPLVS |
| | | | | | | EKKFPEFVQIWEDEVLCLPVPSNLTKSETLMPLYQKTSGK |
| | | | | | | IGLVDRVLRRAAILSLRKGLKNIDKATLDEVLEWFE |
| MG64 | 330 | MG64-68-Q | protein | unknown | uncultivated | MEIPAKEPRFFQVEPLEGESLSHFLGRFRRANYLTATQLG |
| transposition | | transposition | | | organism | KLTGIGAVISRWEKFYLNPFPTPHELEALAAVVEVKVDR |
| proteins | | protein | | | | LSEMLPPKGVTMKPRPIRLCGACYQESPCHRVEWQFKDV |
| | | | | | | MVCDRHNLRLLTKCTNCETPFPIPADWVQGECPHCFLPF |
| | | | | | | ATMAKRQKAC |
| MG64 | 331 | MG64-67-B | protein | unknown | uncultivated | MGKRLNSNEVDESLLLHDDSDEVDEISESEDAKENNVIV |
| transposition | | transposition | | | organism | TELSEEAKLRMQVLQNLVEPCDRKTYGIKLKQAAEKLG |
| proteins | | protein | | | | KTVRTVQRLVKKYQQQGLSGIIEVERSDKGDYRIDDDW |
| | | | | | | QDFIVKTYKEGNKGGRKMTPAQAAIRVQVRAGQLGLDK |
| | | | | | | YPCHMTVYRVLNPIIERKEQKQKVRNIGWRGSRVSHQTR |
| | | | | | | DGQTLDVHHSNHVWQCDHTKLDVMLVDQYGETLARP |
| | | | | | | WLTKITDSYSRCIMGIHLGFDAPSSLVVALAMRHAMLRK |
| | | | | | | QYSSEYKLHCEWGTYGVPENLFTDGGQDFRSEHLKQIGF |
| | | | | | | QLGFECHLRDRPPEGGIEERGFGTINTDFLSGFYGYLGSN |
| | | | | | | VQARPEGAEEEACITLRELHLLIVRYIVDNYNQRIDARSG |
| | | | | | | NQTRFQRWEAGLPALPNLVNERELDICLMKKTRRNIYKG |
| | | | | | | GYVSFENIMYRGDYLSAYAGESVLLRYDPRDISTVFVYR |
| | | | | | | QDSGKEVLLSQAHAIDLETEQISWEEAKAASRKIRNAGK |
| | | | | | | QLSNKSILAEVQDRDTFIKQKKKSHNISDTAGVRSVGCC |
| | | | | | | GGS |
| MG64 | 332 | MG64-67-Q | protein | unknown | uncultivated | MAAVVEVKVDRLIEMLPPKSVTMKPRPIRLCGACYQESP |
| transposition | | transposition | | | organism | CHRVEWQFKDVMVCDRHQLRLLTKCTNCETPFPIPADW |
| proteins | | protein | | | | VQGECPHCLLPFATMARRQKRC |
| MG64 | 333 | MG64-71-C | protein | unknown | uncultivated | MSQDSQNSLVMDTDDEHPQVRYKGKLINSHKLPSNELLT |
| transposition | | transposition | | | organism | DEVNFRMEVIQSLTEPCDRKTYAIRKKEAAEKLGVSIRQ |
| proteins | | protein | | | | VERLLKKWREERLVGLATTRSDKGKYRLDQEWIDFIIDT |
| | | | | | | FKKGNEGSKRMTRHQVFLRVKGRAKQLNLNKGEYPSHQ |
| | | | | | | SVYRILDEYIEEKQRKLKARSPGMLGERLTHMTRDGREL |
| | | | | | | EVECSNDVWQCDHTRLDVRLVDEYGVLDRPWLTIVIDS |
| | | | | | | YSRCLVGFYLGFDNPSSQIDAL |
| MG64 | 334 | MG64-71-Q | protein | unknown | uncultivated | MPYEGESISHFLGRFRREKGNKFSAASGLGDVAGLGAVL |
| transposition | | transposition | | | organism | ARWEKFYFNPFPTHQELEALATVVEVEADRLREMLPPPG |
| proteins | | protein | | | | VGMKHSPIRLCGHCYAESPCHQIEWQFKVTVGCVSEAPL |
| | | | | | | KEARHKLRLLSKCPICEKPFTIPALWVEGHCPRCFTSFAE |
| | | | | | | MAKYQKHY |
| MG64 | 335 | MG64-72-B | protein | unknown | uncultivated | MNLLEASEFSDQQDDRDFSLIYEEEDTSIEEPPIADEIEGL |
| transposition | | transposition | | | organism | GTGKPETTYQPSEQSIACYGVPEDDTAVEFLDRRYALED |
| proteins | | protein | | | | EIILENGEKVRLQLTEEQKLKREVIRSLLEVRHNRKLFSEQ |
| | | | | | | LKEGAKKLNLSTRQVRRIFQDWVDLGITSLQKAPRSDRG |
| | | | | | | KPRRNKYWYDLSVKIYKDGNKGNRSMTRTQVADRIQD |
| | | | | | | QIYEYVKPELPSEVSKLQEAGFSGEALDQELSRLIEERQET |
| | | | | | | CQQQQQEKQEEIAELKTKVSELRKRKLDTQAAHSRIKQL |
| | | | | | | KEQKQAIVAFEFWRTYGQPPCTRTVEIWLKPIEEKNHKA |
| | | | | | | NTSRNPGWHGDTLILRTRKGEAISVTRSNQVWQIDHTKA |
| | | | | | | DVLLVDEDGVEIGRPILTTVIDCHSRCIVGYRLGLKAPSSL |
| | | | | | | VVALALRHAMLPKQYGPEYEMRCQWITHGSPRYIYTDG |
| | | | | | | AADFNFLEHVGDQLGFKAERREQPSQGGIVERPFRTLSEL |
| | | | | | | LSEAPGYTGPNVQKRPKDVAKVVKMTLRELDMMLAGY |
| | | | | | | FADNYNQAADPRTRANPFVPEQSRMARWEAGLKTPPAV |
| | | | | | | IKPRELDICLMQTDERVVYDNGYVRFANLLYKGENLGK |
| | | | | | | HAKDPVFLRFDPRDITLLLVYSRQDGREKFIARAYAVGL |
| | | | | | | EADQLSLEEVKHSAKKLEKAGKQINNIAIREETIRRRKLF |
| | | | | | | ASKQNITRTDRRNAEEAKANPVPERYEEDQHKRVLKPPV |
| | | | | | | EEQPRPVDVPIDVSIALPEPFIDDDDDSEDYAIEVDD |
| MG64 | 336 | MG64-72-C | protein | unknown | uncultivated | MQNQLSEGIATLSQAKESVPQEKLLPALDILSQPKALTQD |
| transposition | | transposition | | | organism | LLLSLIAALSNPQEQVDDLIAIAKEMFAAMACIAEPEPNIL |
| proteins | | protein | | | | RLDQVSSFIDWIQGRLKLRKPGKAIGETGLGKTCACHAC |
| | | | | | | LEEFEPVQHPNQPSERPFVYVQIDENRCAPGRFLQLILIAL |
| | | | | | | RKPTSGNLHQLKQRVKKFFKQYKVQILLVDEAHCLHFD |
| | | | | | | ALKTARSFYDDKDLGVIPILIGTSNRLDTLMEKDEQVNN |
| | | | | | | RFANTFVFEELAGDKFSGVVKIWGEKIIRMQDPLISKDPS |
| | | | | | | KAGGKKRIISLLKTGKAGINKDLASTLEEMTSGELRLLDN |
| | | | | | | ILRDAAVRLLEKKLGEIYSLVLQALKTDSKVLAFDAKKIL |
| | | | | | | KEIRIDKSFLQSMKGEYLRGRSM |
| MG64 | 337 | MG64-72-Q | protein | unknown | uncultivated | MQFEPHRIELERVEPFPGESISHFLGRFERANVWTTYQIG |
| transposition | | transposition | | | organism | RVTGIKAGVSRWKKLYLSPLFPTKQELEVLAELVEVSAD |
| proteins | | protein | | | | RLAEMLPSESQAMKPPGTIYLCAACYTEMPCHLIQWQFK |
| | | | | | | GVNACDRHRLYLLSKCPKCKKTFSAPADWEEGICFHCG |
| | | | | | | KSFFEMVVHQGRVKNAK |
| MG64 | 338 | MG64-85-B | protein | unknown | uncultivated | MEASQVACLDTDLQAMDEVMLSDRAFDTDPLKILIESAE |
| transposition | | transposition | | | organism | PQKLRFQLIQWLAESPNRTVKAERKEVIVQTLAISTRQVE |
| proteins | | protein | | | | RMLNQYQEDRLRETTGIERSDKGQHRIHNYWQNYIREV |
| | | | | | | YEKSLKDKHPLKPADVVREVQRHAVIDLRHEEGDYPHP |
| | | | | | | ATIYRILKPLVERQKRRQKIRNPGSGSWLAVETRDGKLLR |
| | | | | | | ADYSNQIIQCDHTKLDIRIADKDRKLLNWRPWLTTVVDT |
| | | | | | | FSSGLIGYHLWHKQPGAHEVALTLRQAILPKRFPPDYAIS |
| | | | | | | KPWCYGPPLQYFFTDGGKDLSKSKLIKAIGKKLGFRCEL |
| | | | | | | RDRPTQGGIVERLFQTINTEFLQCLPGYISKEEGGAERAE |
| | | | | | | KEACLTFEDIDKFLAGYFCEYNHDLYPKDESETRFERWF |
| | | | | | | RGMGKQLPEPLDERALDICLMKEEQRVVQAHGSIYFENL |
| | | | | | | TYRCEELRSLKGEYVTLSYDPDHVLTLYIYRQATSDDAV |
| | | | | | | EFIGYAHAINMDTQDLSLDELKQLNKVRSNARREHSNYD |
| | | | | | | ALLALDTRQKLVEQRKQEKKERQRSEQRKLREKSKQNS |
| | | | | | | NVVDMRKARAGKSTSRAEPMELLPERISSEQLKLQSPVPI |
| | | | | | | PGISEPAGLLTEERHNLVSASSQTSKIAVPPPQISETAASAP |
| | | | | | | SSEERHRLIIPKNQTLKRIW |
| MG64 | 339 | MG64-85-C | protein | unknown | uncultivated | MAQSELAVQVPVEVLAPQLDITDVIAKTAAIEELFKTAFI |
| transposition | | transposition | | | organism | PTDRASQYFRWLDELRLLKQCGRVIGPKGVGKSRSSKHY |
| proteins | | protein | | | | REEDRKRVSHIAAWSNSSSKKLFSQILKDINHAAPRVRRH |
| | | | | | | DLRDRLAGCLEPFGIELLLVDNAENLQREALVDLKQLHE |
| | | | | | | ESGVPVILIGEQDLDNNLENADLLTCFPTLFEFDKLDYED |
| | | | | | | LRKTLRTIELDLLALPQASNLAEGNLLEILAVSTQAHMGT |
| | | | | | | LIKLLTKVVLHSLNKGHMKVDEAILRNIASRYGKRYISPE |
| | | | | | | ARKKPELGEG |
| MG64 | 340 | MG64-85-Q | protein | unknown | uncultivated | VSQPEANEPRLGSVEPLEHESISHYLGRLRRLKANSLPSA |
| transposition | | transposition | | | organism | YALGQAAGIGGITVRWEKLYFNPFPTEAGLGAIARLIGLD |
| proteins | | protein | | | | TRRLQAMLPSQGMTLQPRPIRLCGACYAEMPCHQMSWQ |
| | | | | | | HKDIVAVCPHHPLRLLERCPSCKKPFQIPALWMDGKCHH |
| | | | | | | CGIWFTAMVKYQERIKKTG |
| MG64 | 341 | MG64-86-Q | protein | unknown | uncultivated | MAGLGGAIARWEKFYHNPFPTLQQLEALATVIGVEADK |
| transposition | | transposition | | | organism | LANMLPPAGVGIKHEPTRLCGACYAEFACHRIEWQFKTT |
| proteins | | protein | | | | NRCERHQLRLLSECPNCKARFKVPALWVDGHCQRCFMK |
| | | | | | | FAEMAKYQKPVLQ |
| MG64 | 342 | MG64-49-C | protein | unknown | uncultivated | MSQTHLASVPDPSINNRSATQQSPQLWQKPIRSPEIQAEV |
| transposition | | transposition | | | organism | ERIGTIDPYVAVGRDEALFTCLNNWRDRRTCGRIMTVDR |
| proteins | | protein | | | | LGLFKDLDYYTNQQTRRRGDLIRMPAPVAYIEIDDPGNG |
| | | | | | | KNVFLSILDFLANPVSCGNPRDLRLRTWATIKKCGVKILV |
| | | | | | | VNYADLLLFSGLNELMRISEKCGISVILCGTSRLDEILDAK |
| | | | | | | HRKRYLPIHNTFLNHHKLSVLSASEIATVIEQWENTLGCS |
| | | | | | | KSMGLATDERIVKILNQLSDGQIQPLYDLLKRISIWHLDN |
| | | | | | | PHVEINVNNVSTLLTTVQAPQVGLE |
| MG64 | 343 | MG64-49-Q | protein | unknown | uncultivated | MTEPTAECFSIALQPYEDESMSHYLGRWKRQDVVSLSSI |
| transposition | | transposition | | | organism | GSLSRQLRLGTAMSRWEKFYLNPFPTLKQLEQLGKVMGI |
| proteins | | protein | | | | EGERLLLMFPPKGEPINVRIIRLCCACYDEAHYHRMRWQ |
| | | | | | | FQSTAACDRHQLRLLYKCPNCEQHLPIPAEWESGRCKKC |
| | | | | | | QMLFRSMIKHQKPARTEGEKS |
| MG108 | 344 | MG108-1-Q | protein | unknown | uncultivated | VETQTEQALPWQIQPYEGESISHYLGRVRRADAISASSAS |
| transposition | | transposition | | | organism | GLSKALGLGIALARWEKLRFNPYPSRVELEALDKVVGLG |
| proteins | | protein | | | | VDRLADLFPPKDEPIRLQPIRFCPACYVEFPYHRMKWQY |
| | | | | | | QSTAGCDRHHLKLLCKCPGCKDYFQVPSLWTEPKCKRC |
| | | | | | | GMPFKRMVKHQKPHEGD |
| MG64 active | 345 | MG64-6-B- | protein | artificial | | MKKLFAQDVNIDTEVISNQIPTSDPSQSNLIASELPEEARP |
| transposition | | NLS active | | sequence | | KLEVIQSLLEPCDRVTYGERLREGAEKLGLSVRSVQRLFK |
| protein | | transposition | | | | KYQEKGLIALLSGSRTDKGEHRISELWQNFIVKTYQEGN |
| | | protein | | | | KGSKRMSPKQVALKVQAKAGAIADDNPPSYRTVLRVLK |
| | | | | | | PILEKQEKAKSIRSPGWRGSTLSVKTRDGDDLDISYSNQV |
| | | | | | | WQCDHTRADVLLVDQHGKLLVRPWLTTVIDSYSRCIMG |
| | | | | | | INLGFDAPSSQVVALALRHAILPKRYGTEYKLNCDWGTY |
| | | | | | | GTPEYLFTDGGKDFRSNHLAEIGLQLGFVCKLRDRPSEG |
| | | | | | | GIVERPFKTLNQSLFSTLPGYTGSNVQERPEDAEKDAQLT |
| | | | | | | LRDLEQLIVRFIVDRYNQSIDARMGDQTRYQRWEAGLQ |
| | | | | | | KEPDVISERDLDICLMKMSRRTVQRGGHLQFENVMYLG |
| | | | | | | EYLAGYAGEVVSFRYDPRDITTIWVYRQENDREVFLTRA |
| | | | | | | HAQGLETEQLSVDDAKASAKRLRAAGKTISNQSILQETIE |
| | | | | | | REVLAERTKSRKHRQKEEQSYKRSPSAAVMVEVESEQLE |
| | | | | | | IESSNEANANSVSADIEVWDYDEMREGLGWGGGGSGGG |
| | | | | | | GSYPYDVPDYAGSGSPKKKRKVDGSPKKKRKVDSG |
| MG64 active | 346 | NLS-MG64- | protein | artificial | | MKRPAATKKAGQAKKKKYPYDVPDYAGGGGSGGGGSG |
| transposition | | 6-B active | | sequence | | GGGSKKLFAQDVNIDTEVISNQIPTSDPSQSNLIASELPEE |
| protein | | transposition | | | | ARPKLEVIQSLLEPCDRVTYGERLREGAEKLGLSVRSVQ |
| | | protein | | | | RLFKKYQEKGLIALLSGSRTDKGEHRISELWQNFIVKTYQ |
| | | | | | | EGNKGSKRMSPKQVALKVQAKAGAIADDNPPSYRTVLR |
| | | | | | | VLKPILEKQEKAKSIRSPGWRGSTLSVKTRDGDDLDISYS |
| | | | | | | NQVWQCDHTRADVLLVDQHGKLLVRPWLTTVIDSYSRC |
| | | | | | | IMGINLGFDAPSSQVVALALRHAILPKRYGTEYKLNCDW |
| | | | | | | GTYGTPEYLFTDGGKDFRSNHLAEIGLQLGFVCKLRDRP |
| | | | | | | SEGGIVERPFKTLNQSLFSTLPGYTGSNVQERPEDAEKDA |
| | | | | | | QLTLRDLEQLIVRFIVDRYNQSIDARMGDQTRYQRWEAG |
| | | | | | | LQKEPDVISERDLDICLMKMSRRTVQRGGHLQFENVMY |
| | | | | | | LGEYLAGYAGEVVSFRYDPRDITTIWVYRQENDREVFLT |
| | | | | | | RAHAQGLETEQLSVDDAKASAKRLRAAGKTISNQSILQE |
| | | | | | | TIEREVLAERTKSRKHRQKEEQSYKRSPSAAVMVEVESE |
| | | | | | | QLEIESSNEANANSVSADIEVWDYDEMREGLGW |
| MG64 active | 347 | NLS-MG64- | protein | artificial | | MKRPAATKKAGQAKKKKGGGGSGGGGSGGGGSDYKD |
| transposition | | 6-C active | | sequence | | DDDKNATIKENSSQEKPASQIAEELGDFKVDSQLLQIEIA |
| protein | | transposition | | | | RLNKKSIVPLEHIKDLHDWLDEKRKARQSCRLVGESRTG |
| | | protein | | | | KTVACEAYTFRNKPKQEGKQAPTVPVVYIMPPAKCGAK |
| | | | | | | ELFREIIEYLKYRAVRGTVADFRSRAMEVLKGCEVEMIII |
| | | | | | | DEADRLKPETFSDVRDINDKLGIAVVLVGTDRLDAVIKR |
| | | | | | | DEQVYNRFRASRRFGKLTGEDFKRTVEIWEDKVLKMPV |
| | | | | | | ASNLTNKEMLKILLKATEGYIGRLDEILREAAIKSLSRGFR |
| | | | | | | KVEKAVLQEVAREYS |
| MG64 active | 348 | MG64-6 | nucleotide | artificial | | GTCAAAAGCCTCTGAACTGTGTTAAATGGGGGTTAGTT |
| effectors | | effector | | sequence | | TGACTGTTGAAAGACAGTTGTGCTTTCTGACCCTGGTA |
| sgRNA | | engineered | | | | GCTGCCCACCCTGATGCTGCTATCTTTCGGGATAGGAA |
| | | sgRNA 1 | | | | TAAGGTGCGCTCCCAGTAATAGGGGTGTAGATGTACT |
| | | | | | | ACAGTGGTGGCTACTAAATCACCTCCGACCAAGGAGG |
| | | | | | | AATCCATCCGAAAGGATGGGTTGAAAG(N23) |
| MG64 active | | MG64-6 | nucleotide | artificial | | rGTN |
| effectors | | active | | sequence | | |
| single guide | | effector | | | | |
| PAM | | single guide | | | | |
| | | 5′ PAM | | | | |
| MG64 active | 350 | MG64-6 | nucleotide | artificial | | ATAACAGCGCCGCAGGTCATGCCGTCAAAAGCCTGAA |
| effectors | | active | | sequence | | AGGGTTAGTTTGACTGTTGAAAGACAGTTGTGCTTTCT |
| sgRNA | | effector | | | | GACCCTGGTAGCTGCCCACCCTGATGCTGCTATCTTTC |
| | | engineered | | | | GGGATAGGAATAAGGTGCGCTCCCAGTAATAGGGGTG |
| | | sgRNA 2 | | | | TAGATGTACTACAGTGGTGGCTACTAAATCACCTCCGA |
| | | | | | | CCAAGGAGGAATCCAGAAATGGGTTGAAAG(N23) |
| MG64 active | 351 | MG64-6 | nucleotide | artificial | | ATAACAGCGCCGCAGGTCATGCCGTCAAAAGCCTCTG |
| effectors | | active | | sequence | | AACTGTGTTAAATGGGGGTTAGTTTGACTGTTGAAAGA |
| sgRNA | | effector | | | | CAGTTGTGCTTTCTGACCCTGGTAGCTGCCCACCCTGA |
| | | engineered | | | | TGCTGAAAGTAATAGGGGTGTAGATGTACTACAGTGG |
| | | sgRNA 3 | | | | |
| | | | | | | TGGCTACTAAATCACCTCCGACCAAGGAGGAATCCAT |
| | | | | | | CCGAAAGGATGGGTTGAAAG(N23) |
| MG64 active | 352 | MG64-6 | nucleotide | artificial | | ATAACAGCGCCGCAGGTCATGCCGTCAAAAGCCTGAA |
| effectors | | active | | sequence | | AGGGTTAGTTTGACTGTTGAAAGACAGTTGTGCTTTCT |
| sgRNA | | effector | | | | GACCCTGGTAGCTGCCCACCCTGATGCTGTAAGGTGCG |
| | | engineered | | | | CTCCCAGTAATAGGGGTGTAGATGTACTACAGTGGTG |
| | | sgRNA 4 | | | | GCTACTAAATCACCTCCGACCAAGGAGGAATCCAGAA |
| | | | | | | ATGGGTTGAAAG(N23) |
| MG64 active | 353 | MG64-6 | nucleotide | artificial | | ATAACAGCGCCGCAGGTCATGCCGTCAAAAGCCTCTG |
| effectors | | active | | sequence | | AACTGTGTTAAATGGGGGTTAGTTTGACTGTTGAAAGA |
| sgRNA | | effector | | | | CAGTTGTGCTTTCTGACCCTGGTAGCTGCCCACCCTGA |
| | | engineered | | | | TGCTGCTATCTTTCGGGATAGGAATAAGGTGCGCTCCC |
| | | sgRNA 5 | | | | AGTAATAGGGGTGTAGATGTACTACAGTGGTGGCTAC |
| | | | | | | TAAATCACCTCCGACCAAGGAGGAGAAAG(N23) |
| MG64 active | 354 | MG64-6 | nucleotide | artificial | | TGTACAGTGACTAATTATTTGACGTGATGCCAAATTGT |
| transposon | | active | | sequence | | TGTCGCTGATGAAAACTATTGATTTCTCTATATTCTAG |
| end | | transposon | | | | CTGTTTTCCTTGATTAAAGATCGCTAGCCGTTAGTGAC |
| | | end LE 1 | | | | AAATT |
| MG64 active | 355 | MG64-6 | nucleotide | artificial | | TGTACAGTGACTAATTATTTGGTCCTCCCAATCAGTGA |
| transposon | | active | | sequence | | CAATTTAGCTGTCGTCGTTCTCAAAGAAGAGAATTTGA |
| end | | transposon | | | | GGAGTGACAGATTGATTGTCGCTTTCTTTTTTGTAAGC |
| | | end LE 2 | | | | TAGGATAGCATTATGTGTT |
| MG64 active | 356 | MG64-6 | nucleotide | artificial | | TGTACAGTGACTAATTATTTGACGTGATGCCAAATTGT |
| transposon | | active | | sequence | | TGTCGCTGATGAAAACTATTGATTTCTCTATATTCTAG |
| end | | transposon | | | | CTGTTTTCCTTGATTAAAGATCGCTAGCCGTTAGTGAC |
| | | end LE 3 | | | | AAATTAAGTGTCGTCCTCCCAATCAGTGACAA |
| MG64 active | 357 | MG64-6 | nucleotide | artificial | | TGTACAGTGACTAATTATTTGACGTGATGCCAAATTGT |
| transposon | | active | | sequence | | TGTCGCTGATGAAAACTATTGATTTCTCTATATTCTAG |
| end | | transposon | | | | CTGTTTTCCTTGATTAAAGATCGCTAGCCGTTAGTGAC |
| | | end LE 4 | | | | AAATTAAGTGTCGTCCTCCCAATCAGTGACAATTTAGC |
| | | | | | | TGTCGT |
| MG64 active | 358 | MG64-6 | nucleotide | artificial | | TGTACAGTGACTAATTATTTGGCTAGCCGTTAGTGACA |
| transposon | | active | | sequence | | AATTAAGTGTCGTCCTCCCAATCAGTGACAATTTAGCT |
| end | | transposon | | | | GTCGTCGTTCTCAAAGAAGAGAATTTGAGGAGTGACA |
| | | end LE 5 | | | | GATTGATTGTCGCTTTCTTTTTTGTAAGCTAGGATAGC |
| | | | | | | ATTATGTGTT |
| MG64 active | 359 | MG64-6 | nucleotide | artificial | | TGTACAGTGACACATTAATTGTCATCAATGACAGATTG |
| transposon | | active | | sequence | | CTGTCGTGGAGCCAAATTATGTGTCGCTGAGACAAATT |
| end | | transposon | | | | AATGTCGTTTAACTATCAGTGACAAATT |
| | | end RE 1 | | | | |
| MG64 active | 360 | MG64-6 | nucleotide | artificial | | TGTACAGTGACACATTAATTGTCATCAATGACAGATTG |
| transposon | | active | | sequence | | CTGTCGTGGAGCCAAATTATGTGTCGCTGAGACAAATT |
| end | | transposon | | | | AATGTCGTTTAACTATCAGTGACAAATTTTTGTCGCTT |
| | | end RE 2 | | | | TTCACAACAA |
| MG64 active | 361 | MG64-6 | nucleotide | artificial | | TGTACAGTGACACATTAATTGTCATCAATGACAGATTG |
| transposon | | active | | sequence | | CTGTCGTGGAGCCAAATTATGTGTCGCTGAGACAAATT |
| end | | transposon | | | | AATGTCGTTTAACTATCAGTGACAAATTTTTGTCGCTT |
| | | end RE 3 | | | | TTCACAACAA |
| MG64 active | 362 | MG64-6 | nucleotide | artificial | | TGTACAGTGACACATTAATTGTCATCAATGACAGATTG |
| transposon | | active | | sequence | | CTGTCGTGGAGCCAAATTATGTGTCGCTGAGACAAATT |
| end | | transposon | | | | AATGTCGTTTAACTATCAGTGACAAATTTTTGTCGCTT |
| | | end RE 4 | | | | TTCACAACAATAGTGTGAAGGAAGTGCGCCTTTCAATC |
| | | | | | | CATCCTAGAAATT |
| MG64 active | 363 | MG64-6 | nucleotide | artificial | | TGTACAGTGACACATTAATTTCGTTTAACTATCAGTGA |
| transposon | | active | | sequence | | CAAATTTTTGTCGCTTTTCACAACAATAGTGTGAAGGA |
| end | | transposon | | | | AGTGCGCCTTTCAATCCATCCTAGAAATTATAATTCCA |
| | | end RE 5 | | | | ATCCCTACTTACCTAGAATGGTGGTTGAAACTGTAAGA |
| | | | | | | TTCGCGCCGCTAATAAAACTTTCAGCGATTTGGA |
| MG64 | 364 | MG64-9 | nucleotide | artificial | | GTTGCAGGAAGCGATCTGGCGCGAGATAGGATGAAAG |
| effectors | | effector | | sequence | | |
| crRNA | | crRNA | | | | |
| sequence | | sequence | | | | |
| MG64 | 365 | MG64-16 | nucleotide | artificial | | GTTGCATCCGCTTTCCAGCAACCAGGGGGGTGAAAG |
| effectors | | effector | | sequence | | |
| crRNA | | crRNA | | | | |
| sequence | | sequence | | | | |
| MG64 | 366 | MG64-17 | nucleotide | artificial | | GTTGCATCCGCTTTCCAGCAACCAGGGCGGGTGAAAG |
| effectors | | effector | | sequence | | |
| crRNA | | crRNA | | | | |
| sequence | | sequence | | | | |
| MG64 | 367 | MG64-18 | nucleotide | artificial | | GGGGGAATCCTATGCAAGCCATAGTTGCATTGAAAG |
| effectors | | effector | | sequence | | |
| crRNA | | crRNA | | | | |
| sequence | | sequence | | | | |
| MG64 | 368 | MG64-19 | nucleotide | artificial | | GTACCCAAAGCCTTTTTTCCTTAAGCCTATCCGCAC |
| effectors | | effector | | sequence | | |
| crRNA | | crRNA | | | | |
| sequence | | sequence | | | | |
| MG64 | 369 | MG64-21 | nucleotide | artificial | | ATCGCGATCGCCGTCCCAGCTTTGGGCGGGTTGAAAG |
| effectors | | effector | | sequence | | |
| crRNA | | crRNA | | | | |
| sequence | | sequence | | | | |
| MG64 | 370 | MG64-25 | nucleotide | artificial | | GTTTCAACCGCCATCCCAGCTAGGGGTGGGTTGAAAG |
| effectors | | effector | | sequence | | |
| crRNA | | crRNA | | | | |
| sequence | | sequence | | | | |
| MG64 | 371 | MG64-28 | nucleotide | artificial | | GGTTGCATCTGCTTTTCAGCAACTAGGGCGGGGGAAA |
| effectors | | effector | | sequence | | G |
| crRNA | | crRNA | | | | |
| sequence | | sequence | | | | |
| MG64 | 372 | MG64-32 | nucleotide | artificial | | GTTGCATCCGCTTTCCAGCAACCAGGGCGGGTGAAAG |
| effectors | | effector | | sequence | | TT |
| crRNA | | crRNA | | | | |
| sequence | | sequence | | | | |
| MG64 | 373 | MG64-44 | nucleotide | artificial | | GTTGCCTCCCGCTTCGAGGCACGGGAACGATTGAAAG |
| effectors | | effector | | sequence | | |
| crRNA | | crRNA | | | | |
| sequence | | sequence | | | | |
| MG64 | 374 | MG64-46 | nucleotide | artificial | | GTTGCCTCCCGCTTCGAGGCACGGGAACGATTGAAAG |
| effectors | | effector | | sequence | | |
| crRNA | | crRNA | | | | |
| sequence | | sequence | | | | |
| MG64 | 375 | MG64-49 | nucleotide | artificial | | TAAACAAATCTACTACTCAACATAGCTTTGCCAAACG |
| effectors | | effector | | sequence | | |
| crRNA | | crRNA | | | | |
| sequence | | sequence | | | | |
| MG64 | 376 | MG64-57 | nucleotide | artificial | | GTAACAATAACCCTCCCCGTGTAGGGCGGGTTGAAAG |
| effectors | | effector | | sequence | | |
| crRNA | | crRNA | | | | |
| sequence | | sequence | | | | |
| MG64 | 377 | MG64-58 | nucleotide | artificial | | GTTTCAATGCCCCTTCAAGCTTTGGGCGGGTTGAAAG |
| effectors | | effector | | sequence | | |
| crRNA | | crRNA | | | | |
| sequence | | sequence | | | | |
| MG64 | 378 | MG64-59 | nucleotide | artificial | | GTTTCAACGCCGCTTCCAGCTTGAGGCGGGTTGAAAG |
| effectors | | effector | | sequence | | |
| crRNA | | crRNA | | | | |
| sequence | | sequence | | | | |
| MG64 | 379 | MG64-60 | nucleotide | artificial | | GTCGCAATCTGCCTCTCAGAGATGGGTGGGCTGAAAG |
| effectors | | effector | | sequence | | |
| crRNA | | crRNA | | | | |
| sequence | | sequence | | | | |
| MG64 | 380 | MG64-61 | nucleotide | artificial | | GTTTCAACTACCATCCCGACTAGGGGTGGGTTGAAAG |
| effectors | | effector | | sequence | | |
| crRNA | | crRNA | | | | |
| sequence | | sequence | | | | |
| MG64 | 381 | MG64-62 | nucleotide | artificial | | GTTGCATCAGCCCTCCCAGCGTTGGGCGGGTTGAAAG |
| effectors | | effector | | sequence | | AA |
| crRNA | | crRNA | | | | |
| sequence | | sequence | | | | |
| MG64 | 382 | MG64-63 | nucleotide | artificial | | GTTACAATCGCCTGTCCGAATTGGGGCAGGTTGAAAG |
| effectors | | effector | | sequence | | |
| crRNA | | crRNA | | | | |
| sequence | | sequence | | | | |
| MG64 | 383 | MG64-64 | nucleotide | artificial | | GGTTTCAATCGCTCCCATCGATGGGAGCAGGTTGAAA |
| effectors | | effector | | sequence | | G |
| crRNA | | crRNA | | | | |
| sequence | | sequence | | | | |
| MG64 | 384 | MG64-65 | nucleotide | artificial | | GTTTCAACAACCATTCCGGCTAGGGGTGGGTTGAAAG |
| effectors | | effector | | sequence | | |
| crRNA | | crRNA | | | | |
| sequence | | sequence | | | | |
| MG64 | 385 | MG64-66 | nucleotide | artificial | | GTTTCAACAACCATTCCGGCTAGGGGTGGGTTGAAAG |
| effectors | | effector | | sequence | | |
| crRNA | | crRNA | | | | |
| sequence | | sequence | | | | |
| MG64 | 386 | MG64-67 | nucleotide | artificial | | GTTTCAACAACCATCCCGGCTAGGGGTGGGTTGAAAG |
| effectors | | effector | | sequence | | |
| crRNA | | crRNA | | | | |
| sequence | | sequence | | | | |
| MG64 | 387 | MG64-68 | nucleotide | artificial | | CTTTCAACCCACCCCTAGCCGGGATGGTTGTTGAAACT |
| effectors | | effector | | sequence | | |
| crRNA | | crRNA | | | | |
| sequence | | sequence | | | | |
| MG64 | 388 | MG64-69 | nucleotide | artificial | | GTTTCTACTGCCCTCCTGACCTACGGTGGGCTGAAAG |
| effectors | | effector | | sequence | | |
| crRNA | | crRNA | | | | |
| sequence | | sequence | | | | |
| MG64 | 389 | MG64-70 | nucleotide | artificial | | GTTTCATCAACCTTCCCGCTCTTGGGTGGGTTGAAAG |
| effectors | | effector | | sequence | | |
| crRNA | | crRNA | | | | |
| sequence | | sequence | | | | |
| MG64 | 390 | MG64-71 | nucleotide | artificial | | GTAACAATAACCCTCCTGGTGTAGGGTGGGTTGAAAG |
| effectors | | effector | | sequence | | |
| crRNA | | crRNA | | | | |
| sequence | | sequence | | | | |
| MG64 | 391 | MG64-72 | nucleotide | artificial | | GGCGCAATAGCCTTCCTAGACACGGATAAGCTGAAAG |
| effectors | | effector | | sequence | | A |
| crRNA | | crRNA | | | | |
| sequence | | sequence | | | | |
| MG64 | 392 | MG64-73 | nucleotide | artificial | | GTTTCAACAACCATCCCGATACGAGGGTGGGTTGAAA |
| effectors | | effector | | sequence | | GA |
| crRNA | | crRNA | | | | |
| sequence | | sequence | | | | |
| MG64 | 393 | MG64-74 | nucleotide | artificial | | GTCGCAATGGCCGTTTTGGCCGGAGAAGGGATGAAAG |
| effectors | | effector | | sequence | | |
| crRNA | | crRNA | | | | |
| sequence | | sequence | | | | |
| MG64 | 394 | MG64-75 | nucleotide | artificial | | GACGCAATCGCCTTCCCAGAGATGGGTGGGCTGAAAG |
| effectors | | effector | | sequence | | |
| crRNA | | crRNA | | | | |
| sequence | | sequence | | | | |
| MG64 | 395 | MG64-76 | nucleotide | artificial | | GTTTCATCAACCCTCCCGCAACAGGGTGGGTTGAAAG |
| effectors | | effector | | sequence | | |
| crRNA | | crRNA | | | | |
| sequence | | sequence | | | | |
| MG64 | 396 | MG64-77 | nucleotide | artificial | | GTTTCATCAGCCCTCCCGCCTATGGGTGGGTTGAAAG |
| effectors | | effector | | sequence | | |
| crRNA | | crRNA | | | | |
| sequence | | sequence | | | | |
| MG64 | 397 | MG64-78 | nucleotide | artificial | | GTTTCATCAGCCCTCCCGCCTCTGGGTGGGTTGAAAG |
| effectors | | effector | | sequence | | |
| crRNA | | crRNA | | | | |
| sequence | | sequence | | | | |
| MG64 | 398 | MG64-79 | nucleotide | artificial | | GTTTCAACGACCATCCCAGCTAGGGGTGGGTTGAAAG |
| effectors | | effector | | sequence | | |
| crRNA | | crRNA | | | | |
| sequence | | sequence | | | | |
| MG64 | 399 | MG64-80 | nucleotide | artificial | | GTTGCAACAGCCCTCTCAGAGATGCGTGGGTTGAAAG |
| effectors | | effector | | sequence | | |
| crRNA | | crRNA | | | | |
| sequence | | sequence | | | | |
| MG64 | 400 | MG64-81 | nucleotide | artificial | | GTTTCATCAGCCCTCCCGCCTTGGGGTGGGTTGAAAGA |
| effectors | | effector | | sequence | | |
| crRNA | | crRNA | | | | |
| sequence | | sequence | | | | |
| MG64 | 401 | MG64-82 | nucleotide | artificial | | GGCGCAACAGCCCTTTTAGGCATGGGTGAGTTGAAAG |
| effectors | | effector | | sequence | | |
| crRNA | | crRNA | | | | |
| sequence | | sequence | | | | |
| MG64 | 402 | MG64-83 | nucleotide | artificial | | GTTTCAACGACCATCCCAACTAGGGGTGGGTTGAAAG |
| effectors | | effector | | sequence | | |
| crRNA | | crRNA | | | | |
| sequence | | sequence | | | | |
| MG64 | 403 | MG64-84 | nucleotide | artificial | | GTCGCCATCGACTTCCTGGCAACAGGTGGGTTGAAAG |
| effectors | | effector | | sequence | | |
| crRNA | | crRNA | | | | |
| sequence | | sequence | | | | |
| MG64 | 404 | MG64-85 | nucleotide | artificial | | CGCCATCGCCCTCCCAGAGATGGGCGGGCTGAAAG |
| effectors | | effector | | sequence | | |
| crRNA | | crRNA | | | | |
| sequence | | sequence | | | | |
| MG64 | 405 | MG64-86 | nucleotide | artificial | | GTTTCAACGACCATCCCGACTAGGGGTGGGTTGAAAG |
| effectors | | effector | | sequence | | |
| crRNA | | crRNA | | | | |
| sequence | | sequence | | | | |
| MG64 | 406 | MG64-87 | nucleotide | artificial | | GTTTCATCGACCCTCCCGCCTTTGGGTGGGTTGAAAG |
| effectors | | effector | | sequence | | |
| crRNA | | crRNA | | | | |
| sequence | | sequence | | | | |
| MG64 | 407 | MG64-88 | nucleotide | artificial | | GTTGCGATCGCCTTTTCAGCTCGATGAGGGTTGAAAGA |
| effectors | | effector | | sequence | | T |
| crRNA | | crRNA | | | | |
| sequence | | sequence | | | | |
| MG64 | 408 | MG64-89 | nucleotide | artificial | | GTTGCATCCGCTTTCCAGCAACCAGGGCGGGTGAAAG |
| effectors | | effector | | sequence | | |
| crRNA | | crRNA | | | | |
| sequence | | sequence | | | | |
| MG64 | 409 | MG64-90 | nucleotide | artificial | | GTTTCAATGGCCATCTCGATTAGGGGTGGGTTGAAAG |
| effectors | | effector | | sequence | | A |
| crRNA | | crRNA | | | | |
| sequence | | sequence | | | | |
| MG64 | 410 | MG64-91 | nucleotide | artificial | | GTTTCAATGACCCTCCCGAATGGGGGTAGGTTGAAAG |
| effectors | | effector | | sequence | | |
| crRNA | | crRNA | | | | |
| sequence | | sequence | | | | |
| MG64 | 411 | MG64-92 | nucleotide | artificial | | GTCGCAACTGCCCCTTCATTCGAAGGGAGGTTGAAAG |
| effectors | | effector | | sequence | | |
| crRNA | | crRNA | | | | |
| sequence | | sequence | | | | |
| MG64 | 412 | MG64-93 | nucleotide | artificial | | GGTCGCCATCAGCGATTCAGCCGGTGGTGGATAGAAA |
| effectors | | effector | | sequence | | G |
| crRNA | | crRNA | | | | |
| sequence | | sequence | | | | |
| MG64 | 413 | MG64-94 | nucleotide | artificial | | GTCGCAACGGGCTTTTACCGTCTGTGAGGATTGAAAC |
| effectors | | effector | | sequence | | |
| crRNA | | crRNA | | | | |
| sequence | | sequence | | | | |
| MG64 | 414 | MG64-95 | nucleotide | artificial | | GTTGCCTCCCGCTTCGAGGCACGGGAACGATTGAAAG |
| effectors | | effector | | sequence | | |
| crRNA | | crRNA | | | | |
| sequence | | sequence | | | | |
| MG64 | 415 | MG64-96 | nucleotide | artificial | | GTCGCAATCCTCTGCGCGGGTGTGGGGATGTTGAAAG |
| effectors | | effector | | sequence | | |
| crRNA | | crRNA | | | | |
| sequence | | sequence | | | | |
| MG64 | 416 | MG64-97 | nucleotide | artificial | | GTTTCAACACCCCTCCCAGCTAGAGGGGGTTGAAAG |
| effectors | | effector | | sequence | | |
| crRNA | | crRNA | | | | |
| sequence | | sequence | | | | |
| MG64 | 417 | MG64-2 | nucleotide | artificial | | AATTAATAGCGCCGCCGTTCATGCTTCTAGGAGCCTCT |
| effectors | | effector | | sequence | | GAAAGGTGACAAATGCGGGTTAGTTTGGCTGTTGTCA |
| putative | | tracrRNA | | | | GACAGTCTTGCTTTCTGACCCTGGTAGCTGCCCACCCC |
| tracrRNA | | sequence | | | | GAAGCTGCTGTTCCTTGTGAACAGGAATTAGGTGCGCC |
| | | | | | | CCCAGTAATAAGGGTATGGGTTTACCACAGTGGTGGC |
| | | | | | | TACTGAATCACCTCCGAGCAAGGAGGAACCCACT |
| MG64 | 418 | MG64-6 | nucleotide | artificial | | ATAACAGCGCCGCAGGTCATGCCGTCAAAAGCCTCTG |
| effectors | | effector | | sequence | | AACTGTGTTAAATGGGGGTTAGTTTGACTGTTGAAAGA |
| putative | | tracrRNA | | | | CAGTTGTGCTTTCTGACCCTGGTAGCTGCCCACCCTGA |
| tracrRNA | | sequence | | | | TGCTGCTATCTTTCGGGATAGGAATAAGGTGCGCTCCC |
| | | | | | | AGTAATAGGGGTGTAGATGTACTACAGTGGTGGCTAC |
| | | | | | | TAAATCACCTCCGACCAAGGAGGAATCCATCCTTAATT |
| | | | | | | TTTT |
| MG64 | 419 | MG64-18 | nucleotide | artificial | | CTAAAAATCGTGCCGTAGACTATGTGCAAACCTCTGGT |
| effectors | | effector | | sequence | | CTGCGAAAAATAAGACTTAGTTTGGGAAGCTTGATGTT |
| putative | | tracrRNA | | | | TCCCTTGCTTTCTGGGTCTAGCGACTAACCACTCCGAA |
| tracrRNA | | sequence | | | | GCTGCTGCTAGTAAGCGGTGTTCCCACTGGACACAAGT |
| | | | | | | GAATCTGGCAGAATAGGGGCACACCCAGCAAGAGAG |
| | | | | | | GACAGACTACTGTAGTGTTGGTTGCTGCTTCACCCCCG |
| | | | | | | ATCAAGGGGGAATCCTAT |
| MG64 | 420 | MG64-21 | nucleotide | artificial | | AATAGCGCCGCAGTTCATGCTTCTTTGAAGCCTCTGTG |
| effectors | | effector | | sequence | | CTGTGCAAAATGTGGGTTAGTTTGGCTGTTGAAGAAAC |
| putative | | tracrRNA | | | | AGCCTTGCTTTCTGACCCTGGTAGCTGTCCACCCTGAA |
| tracrRNA | | sequence | | | | GCTGCTATCCCCTGTGGATAGGATAGGTGCGCCCCCAG |
| | | | | | | CAATAGGGGAGCGGGTATACCGCAGTGGTGGCTACTG |
| | | | | | | AATCACCTCCAAGCAAGGAGGAATCCACTTTAT |
| MG64 | 421 | MG64-22 | nucleotide | artificial | | AATAGCGCCGCAGTTCATGCTTCTTTGAAGCCTCTGTG |
| effectors | | effector | | sequence | | CTGTGCAAAATGTGGGTTAGTTTGGCTGTTGAAGAAAC |
| putative | | tracrRNA | | | | AGCCTTGCTTTCTGACCCTGGTAGCTGTCCACCCTGAA |
| tracrRNA | | sequence | | | | GCTGCTATCCCCTGTGGATAGGATAGGTGCGCCCCCAG |
| | | | | | | CAATAGGGGAGCGGGTATACCGCAGTGGTGGCTACTG |
| | | | | | | AATCACCTCCAAGCAAGGAGGAATCCACTTTAT |
| MG64 | 422 | MG64-27 | nucleotide | artificial | | AATAGCGCCGCAGTTTAAGCTCAGCAAGCCTCTGGAC |
| effectors | | effector | | sequence | | TGCGAAAAGTATGGGGTAGTTTGACCGTCGGTAAACG |
| putative | | tracrRNA | | | | GTTGTGCTTTCTGCCCCTGGCGACTGCCCACCCCGATG |
| tracrRNA | | sequence | | | | CTGTCGATTTCTTAACTGGGAATCGAGATGAGGTGCGC |
| | | | | | | CCCCAGCAAGAGGGAACGGGTTTACTGGAGTGGTGGT |
| | | | | | | CGCCGAATCACCCCCGAGCAAGGGGGACTCGTCCTTT |
| | | | | | | GC |
| MG64 | 423 | MG64-28 | nucleotide | artificial | | AATAGCGCCGCAGTTTAAGCTCAGCAAGCCTCTGGAC |
| effectors | | effector | | sequence | | TGCGAAAAGTATGGGGTAGTTTGACCGTCGGTAAACG |
| putative | | tracrRNA | | | | GTTGTGCTTTCTGCCCCTGGCGACTGCCCACCCCGATG |
| tracrRNA | | sequence | | | | CTGTCGATTTCTTAACTGGGAATCGAGATGAGGTGCGC |
| | | | | | | CCCCAGCAAGAGGGAACGGGTTTACTGGAGTGGTGGT |
| | | | | | | CGCCGAATCACCCCCGAGCAAGGGGGACTCGTCCTTT |
| | | | | | | GC |
| MG64 | 424 | MG64-44 | nucleotide | artificial | | TCTAGCGCCGCAGCTCATGTCAGCAATGGCCAATGTGT |
| effectors | | effector | | sequence | | TGTGCTAAATGCGAGCTAGTTTGACTGCCTGCTAAGCA |
| putative | | tracrRNA | | | | GTCTTGCTTTCTGGCTCAGGTGACTATCCACCCAAAGG |
| tracrRNA | | sequence | | | | TCGTTGGTGCGCTGGCGATTTGAGGGCACGGGTTCCGG |
| | | | | | | AGTGATAGTTACCATTACACCTCCGGCCAAGGAGGAA |
| | | | | | | TCCACCCCACCCCC |
| MG64 | 425 | MG64-46 | nucleotide | artificial | | TCTAGCGCCGCAGCTCATGTCAGCAATGGCCAATGTGT |
| effectors | | effector | | sequence | | TGTGCTAAATGCGAGCTAGTTTGACTGCCTGCTAAGCA |
| putative | | tracrRNA | | | | GTCTTGCTTTCTGGCTCAGGTGACTATCCACCCAAAGG |
| tracrRNA | | sequence | | | | TCGTTGGTGCGCTGGCGATTTGAGGGCACGGGTTCCGG |
| | | | | | | AGTGATAGTTACCATTACACCTCCGGCCAAGGAGGAA |
| | | | | | | TCCACCCCACCCCC |
| MG64 | 426 | MG64-49 | nucleotide | artificial | | GATTGCGCCTCGATCGATGCTCTATGAGCCGCTCGATC |
| effectors | | effector | | sequence | | GTAGAAAAATGGGTGAGTTTGATTATCTACTTCGTTAG |
| putative | | tracrRNA | | | | ATAATGCTGCTTTCCGACCCTGGCATTCTGTCCGCCCT |
| tracrRNA | | sequence | | | | TGAAGCTGCTTCTCATGGACTAGCGTAAGCTCGTTGGT |
| | | | | | | AAGAAGGAAAAGTCATAATTTAAAGTCACGTCTTTCT |
| | | | | | | AGTATGACATAGGTGCGCTCCCACGCAATATAGGGTT |
| | | | | | | CAGCTTTTATTTTATAAAAGTAGAGACTTTCCTCTAGT |
| | | | | | | GACAGTGCCGAAA |
| MG64 | 427 | MG64-50 | nucleotide | artificial | | CTATTAATCGCGCCGCGTTGAATGTTAGCAATAACCGC |
| effectors | | effector | | sequence | | TCCAACGTGTTAAATGAGGGTTTGTTTGACAGTA |
| putative | | tracrRNA | | | | |
| tracrRNA | | sequence | | | | |
| MG64 | 428 | MG64-53 | nucleotide | artificial | | CTATTAATCGCGCCGCGTTGAATGTTAGCAATAACCGC |
| effectors | | effector | | sequence | | TCCAACGTGTTAAATGAGGGTTTGTTTGACAGTA |
| putative | | tracrRNA | | | | |
| tracrRNA | | sequence | | | | |
| MG64 | 429 | MG64-57 | nucleotide | artificial | | TAAATAATAGCGCCGCAGTTCATGTTCTTAGGAACCGC |
| effectors | | effector | | sequence | | TGAACTGTGAAAAATCTGGGTTAGTTTGACTATTGGAA |
| putative | | tracrRNA | | | | GATAGTCTTGCTTTCTGACCCTAGTAGCTGCTCACCCC |
| tracrRNA | | sequence | | | | GATGCTGCTGTCTGCGGACAGGAATTAGGTGCGCTCCC |
| | | | | | | AGCAAAAAGGGCGCGGATATACTGCTGTAGTGGCTAC |
| | | | | | | CAAATCACCCTCGACCAAGGGGGAACCCATC |
| MG64 | 430 | MG64-58 | nucleotide | artificial | | TAACAAACAGCGCCGCAGTTCATGCGTCTTATGGCGCC |
| effectors | | effector | | sequence | | TCTGTGCTGTGCAAAATGTGGGTTAGTTTGACTGTTGG |
| putative | | tracrRNA | | | | AAGACAGTCTTGCTTTCTGACCCTGGTAGCTGCCCACC |
| tracrRNA | | sequence | | | | TTGAAGCTGCTATCCCTTGTGGATAGGAATCAGGTGCG |
| | | | | | | CCCCCAGTAATAGAGGTGCGGGTTTACCGCAGTGGTG |
| | | | | | | GCTACCGAATCACCTCCGAGCAAGGAGGAACCCACC |
| MG64 | 431 | MG64-59 | nucleotide | artificial | | TAATCAACAGCGCCGTTGTTCATGCGTTTTTACGCCTC |
| effectors | | effector | | sequence | | TGAGCAATGATAAATTTGGGTTAGTTTGACTGTTAGAA |
| putative | | tracrRNA | | | | ATACAGTTTTGCTTTCTGACCCTGGTAGCTGCCCACCT |
| tracrRNA | | sequence | | | | TGAAGCTGCTATCTCTTGTAGATAGGAAATAAGGTGC |
| | | | | | | GCCCCCAGTAATAGAGGTGCGGGTTTACCGCAGTGGT |
| | | | | | | GAGTCCACTTACCGGGAAAACGTTCTTTTCGGTAAAGT |
| | | | | | | GGCGAATCCGAAGGGTGGCTATCGAATCACCTCCGAT |
| | | | | | | CAAGGAGGAACCCACT |
| MG64 | 432 | MG64-60 | nucleotide | artificial | | TGACTAACAGCGCCGCAGTTCATGCTCCTTGAGCCGCT |
| effectors | | effector | | sequence | | GAACTGTGAAAAATGAGGGGTCAGTTTGGTCGTTGTG |
| putative | | tracrRNA | | | | AGACGATCGTGCTTTCCGCCCCTAAGTAGCTGCCCGGT |
| tracrRNA | | sequence | | | | CACTGACTGCCATCTTGGTCGTTGTTCGATTATGAACA |
| | | | | | | ACTGATGGGGATGGGAAGTTGCGTGGATGAGTTGCTC |
| | | | | | | TTCCAATGTAACGCAGGTGCGCGCCCAGCAGAAGTGA |
| | | | | | | TCCCAGCCTTCAGCAATGAAGGTACAGCCTGGTTCTGT |
| | | | | | | AGTGGCAGCTACTGATTGTCTCCGAGCAAGGAGGAGT |
| | | | | | | CCTCC |
| MG64 | 433 | MG64-62 | nucleotide | artificial | | TCAGCAATAGCGCGCCGTTACCAAACGGTGTTAAGTA |
| effectors | | effector | | sequence | | AAGGGGTCAGTTTAATTGCTTTCCGCCCCGGTAGCTGA |
| putative | | tracrRNA | | | | CATCTCTTCCTATGGATTTCCATGGGTACAATGCAGGG |
| tracrRNA | | sequence | | | | TCGCGCCTAGCATTAAAGGGAAATTCTTATTATTAGTG |
| | | | | | | GTTCCCGCCTCTAATAATAAGGATACAGAAATACTTGT |
| | | | | | | CTGTCGGCTACTAAAAGCCCGAGCAAGGGTCCCCCCG |
| | | | | | | AT |
| MG64 | 434 | MG64-63 | nucleotide | artificial | | TATGTAATAGCGCAGCCGTTCATGTTGTTTACAGCCTC |
| effectors | | effector | | sequence | | TGAACTGTAATGGGTTAGTTTGACTGTTGGAAGACAGT |
| putative | | tracrRNA | | | | CGTGCTTTCTGACCCTGGTAGCTGCTCACCCCGATGCT |
| tracrRNA | | sequence | | | | GCTGTCTCTTGAGACAGGATAGGTGCGCTCCCAGCAAT |
| | | | | | | AAGGGCGCGGATGTACCGCTGTAGTGGCTACCGAACC |
| | | | | | | ACCCCCGATCAAGGGGGAACCCGCT |
| MG64 | 435 | MG64-64 | nucleotide | artificial | | GTTTGAACAGCGCCGCAGTTCATGCTTGCCATCAAGCC |
| effectors | | effector | | sequence | | TCTGTGCTGTGAAAAATATGGGTTAGTTTGGTTATTGG |
| putative | | tracrRNA | | | | AAAATAGCCTTGCTTTCTGACCCTAGTGGCTGCTTACC |
| tracrRNA | | sequence | | | | CCGATGCTGCTGTCTCTTGGAACAGGAATAAGGTGCG |
| | | | | | | CTCCCAGCAAAAAGGGCGCGGGTATACCGCTGTAGTG |
| | | | | | | GCTACTGAATCACTCCCGAGCAAGGGAGAACCTTCT |
| MG64 | 436 | MG64-65 | nucleotide | artificial | | AGATGAACAGCGCCGCAGTTCATGCTCTTTGAGCCAAT |
| effectors | | effector | | sequence | | GTGCTGCGATAAATCTGGGTTAGTTTGACGGTTGGAAA |
| putative | | tracrRNA | | | | ACCGTTATGCTTTCTGACCCTGGTAGCTGCCCGCTTCT |
| tracrRNA | | sequence | | | | GATGCTGCCATCTGTAGAATTCTATAGATGGGATAGGT |
| | | | | | | GCGCTCCCAGCAATAAGGAGTAAGGCTTTTAGCTGTA |
| | | | | | | GCCGTTGTTCTCAACGGTGCGGGTTACCGCAGTGGTGG |
| | | | | | | CTACTGAATCACCCCCTTCGTCGGGGGAACCCTCT |
| MG64 | 437 | MG64-66 | nucleotide | artificial | | AAATAAATAGCGCCGCAGTTCATGCTCTTTGAGCCAAT |
| effectors | | effector | | sequence | | GTACTGTGATAAATCTGGGTTAGTTTGACGGTTGGAAA |
| putative | | tracrRNA | | | | ACCGTTTTGCTTTCTGACCCTGGTAGCTGCTCGCTCTTG |
| tracrRNA | | sequence | | | | ATGCTGCTGTCTGGCTTGACTAGGCAGGATATGCCCTT |
| | | | | | | TTGCCATCTTGGATAACTAAGTTTTTGATGTTTTCACCG |
| | | | | | | ACAAGAAAAAACTTTTATACAAGATATATCAAATAGG |
| | | | | | | GACAGGTGCGCTCCCAGCAATAAAGAGTAAAGCTGTA |
| | | | | | | AAGCTTGAGCCGTTTTATAACGGTGGGGATTACCTCAG |
| | | | | | | TGGCGGTTACTGAATCACCCCCTTCGTCGGGGGAACCC |
| | | | | | | TCT |
| MG64 | 438 | MG64-67 | nucleotide | artificial | | AAACAAACAGCGCCGCAGTTCATGCTCTTCGAGCCAA |
| effectors | | effector | | sequence | | TGTACTGTGATAAATCTGGGTTAGTTTGACGGTTGGAA |
| putative | | tracrRNA | | | | AACCGTTTTGCTTTCTGACCCTGGTAGCTGCCCGCTCTT |
| tracrRNA | | sequence | | | | GATGCTGCTGTCTGGCTTAACTAGGCAGGATATGCCCT |
| | | | | | | TTTGGCATCTTGGATAACTAAGTTTTTGATGTTTTTACC |
| | | | | | | GACAATAAAAAACTTTTATACAAGTATATCAAATAGG |
| | | | | | | GACAGGTGCGCTCCCAGCAATAAAGAGTAAAGCTGCA |
| | | | | | | AAGCTTGAGCCGTTTTATAACGGTGGGGTTTACCTCAG |
| | | | | | | TGGTGGCTACTGAATCACCCCCTTCGTCGGGGGAACCC |
| | | | | | | TCT |
| MG64 | 439 | MG64-68 | nucleotide | artificial | | AAAGTAACAGCGCCGCAGTTCATGCTCTTTTGAGTCTC |
| effectors | | effector | | sequence | | TGTACTGTGATAAATCTGGGTTAGTTTGACGGTTGAAA |
| putative | | tracrRNA | | | | GACCGTTTTGCTTTCTGACCCTGGTAGCTGCTCGCTCTT |
| tracrRNA | | sequence | | | | GATGCTGCTGTCGTAAGACAGGATAGGTGCGCTCCCA |
| | | | | | | GCAATAAAGAGTAAAGCTGATAAAGCTTGAGCCGTTG |
| | | | | | | TAAAACGGTGGGGTTTACCTCAATAATGGCTACTGAAT |
| | | | | | | CACCCCCTTCGTCGGGGGAACCCTCC |
| MG64 | 440 | MG64-69 | nucleotide | artificial | | GACTTCATGGCGCGCTGCTTCGGCAGCTAAAAATACTG |
| effectors | | effector | | sequence | | GGTCAGTTTATTTGCTTTCCGTCCCAGGTAGTTGTCCGT |
| putative | | tracrRNA | | | | TTCTGGTAAGTGATGTAAGCGACATCCTGCCTTGTGCA |
| tracrRNA | | sequence | | | | GGAACATAGCTCACTTCATAGATGTACGGTTGCGCCAC |
| | | | | | | TTTACATCAGGAAGCAGTTTTTGTTACTAGAGCTATTA |
| | | | | | | ACCTAGCAACAAGGATACCGAATTACAGGTGCTGCCT |
| | | | | | | GCCAGTAAATTCTTTCTATTTAATAGAGAGGTGCATTG |
| | | | | | | GTAGGCAGGTGGACAGCTACTAAACGCCCCAAGCAAG |
| | | | | | | GGTTGAGCCTACCCTTTTTCTTCATC |
| MG64 | 441 | MG64-70 | nucleotide | artificial | | TGTTCAATAGCGCGTCTCGTTCCCTGTGAACAGATGAT |
| effectors | | effector | | sequence | | AAGTGTATGGGCAGTTTAATTGCTTTCCGCCCTGTGTA |
| putative | | tracrRNA | | | | GTTGTCCGCGTCTCTTTAAATAATTAGAGAGACACGTC |
| tracrRNA | | sequence | | | | GTTACGGAGCTTGCTGCGTAAATGACGTTCCTTCTGCG |
| | | | | | | GAACTTTTCCGTAGATAGGGTGCAGGGTCGCGCCTAG |
| | | | | | | CATCAGGGAGCTATGTTTTTATAACCAGCGGTTAGCGC |
| | | | | | | CTCTGGTTATAAGGATACAGGTGTACGTGTCGTGGCAG |
| | | | | | | CTACCGAATCGCCTCCGAGCAAGGAGGAGTCCTCC |
| MG64 | 442 | MG64-71 | nucleotide | artificial | | TAAGTAATAGCGCCGCAGTTCATGTTCTTAGGAACCGC |
| effectors | | effector | | sequence | | TGAACTGTGAAAAATCTGGGTTAGTTTGACTATTGGAA |
| putative | | tracrRNA | | | | GATAGTCTTGCTTTCTGACCCTAGTAGCTGCTCACCCC |
| tracrRNA | | sequence | | | | GATGCTGCTGTCTGCGGACAGGAATTAGGTGCGCTCCC |
| | | | | | | AGCAAAAAGGGCGCGGATATACTGCTGTAGTGGCTAC |
| | | | | | | CAAATCACCGCTCCGAAAAACCTATG |
| MG64 | 443 | MG64-73 | nucleotide | artificial | | GTATTAATAGCGCCGCAGATTATCTTTATAACTGCGAA |
| effectors | | effector | | sequence | | AATTATGGGTTAGTTTGACCGTCGGTAGGCGGTTGTGC |
| putative | | tracrRNA | | | | TTTCTGGCCCTTGTAGCTGTCCACCCTGATGCCGATCT |
| tracrRNA | | sequence | | | | CTATACTTCTGGAATAGGGATGATTAACCCGAGAGAT |
| | | | | | | GAGGTTCTTAGATACTTCAATTCTATGGGGTAGGTGCG |
| | | | | | | CCCCCGGCAATAAGTGGCGTGGGTTTACCACAGTGAT |
| | | | | | | GGCTATAAAATCACCTCCGAGCAAGGGGGAATCCACT |
| MG64 | 444 | MG64-74 | nucleotide | artificial | | AGCCACATAGTTCATAAGCTCACGCTTCTTGGACTTCC |
| effectors | | effector | | sequence | | TGTGTTCTCTAAAACGGGTTCTGTTTTACCCTTACCAA |
| putative | | tracrRNA | | | | GGGATACTTTCAGATCCGAGTAGCTGCAAGCTCATGG |
| tracrRNA | | sequence | | | | CGGAGTGTCCCCTGACGCTTTGCCACCGTCATAGCGAT |
| | | | | | | GTGATGGCCGTCTGGCGTATGAACGATATTGAGGGTG |
| | | | | | | AGGTTGATTCCTAAAGCCGATCATACAGCGCAACCAG |
| | | | | | | GTGAATGAGTAAGATTCCGCAAGGATCTAAACCTTAA |
| | | | | | | GCAGTCTTCTGCTGAGGTTGGGCATGGTTCTCAGTGTG |
| | | | | | | GCTACTGACCTTCTTGATCTTTT |
| MG64 | 445 | MG64-75 | nucleotide | artificial | | GAAACAATAGCGCCGCAGTTCAAGCTCTTTGAGCCGC |
| effectors | | effector | | sequence | | TGAACTGTGAAAAATGAGGGTCAGTTTGGTCGTCGCA |
| putative | | tracrRNA | | | | AGACAACTGTGCTTTCCGACCCTGGTAGCTGTCCGCTC |
| tracrRNA | | sequence | | | | ACTGACTGCCGTCCTGACGGAGACTTCTTTTTGAGGTT |
| | | | | | | TATGTGGGGATGGGAAGCTGCATTAGTTGAGTCCGTTC |
| | | | | | | TTCAAATGTAGCGCAGGTGCGCACCCAGCAGAAGTGA |
| | | | | | | GTCAAGCCTTTATCGATAGGGGGTACAGGAGCATCAT |
| | | | | | | CACTTCGATTTATTGATGGTGATGGAGTGTGACTGAAG |
| | | | | | | TGGCAGCTACTGAATCGCCTCCGCTCAAGGAGGAGTC |
| | | | | | | CTCC |
| MG64 | 446 | MG64-76 | nucleotide | artificial | | AAATTAACAGCGCCGACCCTTCATGCTCTTCGGAGCCA |
| effectors | | effector | | sequence | | ATGTAGGTGAAAAATGGGTTAGTTTGACGGTTGGAAA |
| putative | | tracrRNA | | | | ACCGTTTTGCTTTCTGACCCTGGTAGCTGCCCGCTTCTG |
| tracrRNA | | sequence | | | | ATGCTGCCGTCTATCGAATTGCTCGTCCAGACGGGAAA |
| | | | | | | TCTTAGCTCTAAATATCTAAATAGTGTCTTACTTCTAG |
| | | | | | | GATATCCAGAGATAAGAGAGGTGCGCTCCCAGCAATA |
| | | | | | | AGGAGTAATGCTTAACTTGCACTAGCCCTTGGTAACAA |
| | | | | | | GGGTGCGGATAACCGCAGTGGTGGCTACTGAATCACC |
| | | | | | | CCCTTCATCGGGGGAACCCTCC |
| MG64 | 447 | MG64-77 | nucleotide | artificial | | TATTCAATAGCGCGTCTCGTTCCCTGAGAACAGACGAT |
| effectors | | effector | | sequence | | AAGTGTAAGGGCAGTTTAACTGCTTTCCGCCCTTGGTA |
| putative | | tracrRNA | | | | GTTGTCCGCTTCTCTCGCTTAAATTGGAGAGAGACGTT |
| tracrRNA | | sequence | | | | CTTTACGGAGCTTGCTCTGTAAGCGACGTTCCTTTTAC |
| | | | | | | GGAAGTTTTCCGTGAATACGGTGCAGGGTCGCGCCTA |
| | | | | | | GCATCAAGGGGCAATGTTTTTATAACAGTGGTAAAGC |
| | | | | | | ACCTCTGGTTATAAGGATACAGGGTTACGTGTCGTGGC |
| | | | | | | AGCTACCCAATCGCCTTCGAGCAAGAAGGAATCCTCC |
| MG64 | 448 | MG64-78 | nucleotide | artificial | | ATGTCAATAGCGCGTCTTGTTCCCTGAGAACATGACAA |
| effectors | | effector | | sequence | | TACAGGGCAGTTTAATTGCTTTCCGCCCTTGGTAGTTG |
| putative | | tracrRNA | | | | TCCGCTTCTCTCGCTTAAATTGGAGAGAGACGTTCTTT |
| tracrRNA | | sequence | | | | ACGGAGCTTGCTCTGTAAGTGACGTTCCTTCTACGGAA |
| | | | | | | TTTTCTCTGTAGATACGGTGCAGGGTCGCGCCTAGCAT |
| | | | | | | CAAGGGGCAATGTTTTTATAACAGTGGTTCGCACCTCT |
| | | | | | | GGTTATAAGGCGACAGGAGTACGTGTCGTGGCAGCTA |
| | | | | | | CCCAATCGCCTTCGAGCAAGAAGGAATCCTCC |
| MG64 | 449 | MG64-79 | nucleotide | artificial | | TATCAAATAGCGCCGCAGATCATGCAGTAAAAAGCCT |
| effectors | | effector | | sequence | | CTGAACTGTGAAAAATGCGGGTTAGTTTGACTGTTGAA |
| putative | | tracrRNA | | | | AAGCAGTCTTGCTTTCTGACCCTGGTAGCTGCCCACCC |
| tracrRNA | | sequence | | | | CGAAGCTGCTGTTCTAAAAGGACAGGAATTAGGTGCG |
| | | | | | | CCCCCAGTAATAAGGGTGCGGGTATACCGCAGTGGTG |
| | | | | | | GCTACTCAATCACCTCCGAGCAAGGAGGAATCCACC |
| MG64 | 450 | MG64-80 | nucleotide | artificial | | AAGAAAACAGCGCCGCAGTAACATGCTCTTCGGAGCC |
| effectors | | effector | | sequence | | TATGTACTGCGATAAGTGAGGGTCAGGTTTACTGTTGT |
| putative | | tracrRNA | | | | TCAGACAGATGCTTTCCGACCCTGGTAGCTGTCAGCCT |
| tracrRNA | | sequence | | | | GTCGGGTCGCCATCTCGCAGCCAGTGCTGTATGTAGAT |
| | | | | | | GGGACACGGCTGTATCATGAGCTTGCTTTCTCAAGGTA |
| | | | | | | TAGCATAGGTGCGCCCCCGGCTACGATAGGCAACGCT |
| | | | | | | CTACCCCTGGCAACAGTGCGGTACAGATATACACTTGT |
| | | | | | | GGCGGCTACCGAATCGCCTCCGAGCAAGGAGGAACCC |
| | | | | | | TCT |
| MG64 | 451 | MG64-81 | nucleotide | artificial | | ATGTCAGTAGCTCGTCTTGTTCCCTGAGAACATGACAA |
| effectors | | effector | | sequence | | TAAAGGGCAGTTTAATTGCTTTCCGCCCTGGTAGCTGT |
| putative | | tracrRNA | | | | CCGCTTCTCTCGCTTAAATTGGAGAGAGACGTTCTTTA |
| tracrRNA | | sequence | | | | TAGAGCTTGCTCTGTAAGTGACGTTCCTTCTGCGGAAC |
| | | | | | | GTTTCCGTAGATACGATGCAGGGTTGCGCCTAGCATCA |
| | | | | | | AGGGGCAATGTTTTTATAACAGTGGTTCACACCTCTGG |
| | | | | | | TTATAAAGATACAGGAGTACGTGTCGTGGCAGCTACC |
| | | | | | | CAATCGCCTTCGAGCAAGAAGGAATCCTCC |
| MG64 | 452 | MG64-82 | nucleotide | artificial | | ATAATGCTGGCGGCCCAGTTAGTGCTTTAGGCACGAA |
| effectors | | effector | | sequence | | CATGTGGAAAAAGGTCAGTTTTACCCTTGGGTGCTTTC |
| putative | | tracrRNA | | | | CGACCTGTGTACTGTCCGCTATTCATGCTGCTGCCTAA |
| tracrRNA | | sequence | | | | TAAGGCAATTCGCCACAGCAATGAGTAGCACCGCTCT |
| | | | | | | ACCGCCTTAAAAAAGCGGTACAGTAATGCAGATGTGG |
| | | | | | | CAGTCCAAATCGCCTACTGAATACGGTAGGATCTCCC |
| MG64 | 453 | MG64-83 | nucleotide | artificial | | TAATCAACAGCGCCGCAGGTCATGTTCATTTGAACCGC |
| effectors | | effector | | sequence | | TGAACTGCGAACAGTATGGGTTAGTTTGACTGTTGGAA |
| putative | | tracrRNA | | | | GACAGTTGTGCTTTCTGACCCTGGTAGCTGTCCACTCT |
| tracrRNA | | sequence | | | | GATGCTGCCGTTGAAAGACGGGAATAAGGTGCGCTCC |
| | | | | | | CAGCAATAGGGGTGTAGACATACTACAGTGATGGCTA |
| | | | | | | CCGAATCACCTCCGAGCAAGGAGGAATCCACT |
| MG64 | 454 | MG64-85 | nucleotide | artificial | | GGAACAATAGCGCCGCAGTTCAAGCTCTTTGAGCCGC |
| effectors | | effector | | sequence | | TGAACTGTGAAAAATGAGGGTCAGTTTGGTCGTCGTG |
| putative | | tracrRNA | | | | AGACAACTGTGCTTTCCGACCCTGGTAGCTGTCCGCTC |
| tracrRNA | | sequence | | | | ACTGACTGCCGTCTTGGCACAGACTCCATTTTGAGGTT |
| | | | | | | TGTGTGGGGATGGGAAGCTGCATTCGTTGAGTCCGTTC |
| | | | | | | TTCAAATGTAGCGCAGGTGCGCGCCCAGCAGAAGTGA |
| | | | | | | GTTCAGCCTTCACTGTATGAAGGTACAGGAGCATCATC |
| | | | | | | ACTT |
| MG64 | 455 | MG64-86 | nucleotide | artificial | | TAGTAAATAGCGCCGCTGGTCATGCTTGCAAAAGCCTC |
| effectors | | effector | | sequence | | TGAACAGTGATAAATGAGGGTTAGTTTGACTATGGGA |
| putative | | tracrRNA | | | | ACATGGTCTTGCTTTCTGGCCCTGGTAGCTGCCCACCC |
| tracrRNA | | sequence | | | | CGATGCTGCTGTCCCTTGCGGACAGGAATTAGGTGCGC |
| | | | | | | CCCCAGTAATAAGGGTGCGGGTTTACCGCAGTGGTGG |
| | | | | | | CTACTCAATCACCTCCGACCAAGGAGGAACCCACC |
| MG64 | 456 | MG64-87 | nucleotide | artificial | | TATTCAATAGCGCGTCTCGTTCCCCGTGAACAGATGAT |
| effectors | | effector | | sequence | | AAGTGTCGGGGCAGTTTAATTGCTTTCCGCCCTTGGTA |
| putative | | tracrRNA | | | | GTTGTCCGCGTCTCTCCAATCTGTAGAGAAGAGAGAC |
| tracrRNA | | sequence | | | | ACGTCGGGAGCAGAGCTTGCTCTGTAATCGACGTTCCT |
| | | | | | | TCTACGAAACTCTTTCGTAGATATGGTGCAGGGTCGCG |
| | | | | | | CCTAGCATCAGGGAGCTATGTTTTTATAACCTTGGTTT |
| | | | | | | AGAGCCTCTGGTTATAAGGATACAGGTTTACGTGTTGT |
| | | | | | | GGCAGCTACCGAATCGCCTTCGAGCAGGAAGGACCTT |
| | | | | | | CCC |
| MG64 | 457 | MG64-88 | nucleotide | artificial | | TATTCAATAGCGCACCGTTCTCTAAGAACAGGTGACA |
| effectors | | effector | | sequence | | ATTAAGGGGCAGTTTAATTGCTTTCCGTCCCAGGTAGT |
| putative | | tracrRNA | | | | TGTCCTCTCTTTTCATAGGCTTACCTATGAATGGGTGC |
| tracrRNA | | sequence | | | | AGGGTCGCGCCTAGCATCAGAGAGCTATGTTTTCATAG |
| | | | | | | GGTGGTTAAGCGCCACTACTATGAGGATACAGGAATA |
| | | | | | | CGTGTAATCGGGCTGCTACCAAACCGCCTCCGAGCAA |
| | | | | | | GGAGGAACCCCTT |
| MG64 | 458 | MG64-89 | nucleotide | artificial | | GTGAAAATAGCGCCGCAGTTTAAGCTCAGCAAGCCTC |
| effectors | | effector | | sequence | | TGGACTGCGAAAAGTATGGGGTAGTTTGACCGTCGGT |
| putative | | tracrRNA | | | | AAACGGTTGTGCTTTCTGCCCCTGGCGACTGCCCACCC |
| tracrRNA | | sequence | | | | CGATGCTGTCGATTTCTTAACTGGGAATCGAGATGAGG |
| | | | | | | TGCGCCCCCAGCAAGAGGGAACGGGTTTACTGGAGTG |
| | | | | | | GTGGTCGCCGAATCACCCCCGAGCAAGGGGGACTCGT |
| | | | | | | CC |
| MG64 | 459 | MG64-90 | nucleotide | artificial | | TAATTAATAGCGCCGCCGGTCATGCTTGCAAGAACCTC |
| effectors | | effector | | sequence | | TGATCGGTGATAAATGAGGGTTAGTTTGACGGTTGGA |
| putative | | tracrRNA | | | | AGACCGTTGTGCTTTCTGACCCTGGTAGCTGCCCACCC |
| tracrRNA | | sequence | | | | CGAAGCTGCTATCCCTTGGGGATAGGAATTAGGTGCG |
| | | | | | | CCCCCAGTAATAAGGGTGCGGGTTTACCGCAGTGGTG |
| | | | | | | GCTACTGAATCACCTCCGACCAAGGGGGAACCCACT |
| MG64 | 460 | MG64-91 | nucleotide | artificial | | AGCGTCGCAGTCCATGCTTTTTAATCAAGCCTCTGCAC |
| effectors | | effector | | sequence | | TGTGAAAAAATTGGGTTAGTTTGACTGTCTGGAGATAG |
| putative | | tracrRNA | | | | TCCTTCTTTCTGACCCTGGTAACTACCCGCAACTGAAG |
| tracrRNA | | sequence | | | | CTGCTATCTCTAAGTCTCAGCTAGGAGATAGGACATAC |
| | | | | | | TTAAAATAAAGAACATCGTATCTTTATCTTATTAGGTT |
| | | | | | | AGGTGCGCTCCCAGCAATTAAGTTGTATAGTTTTAAGA |
| | | | | | | CTGGGAAATATCTTAAAACTTAATCCTGCTAGTTCAGG |
| | | | | | | ATGTAGATGACTACAGTGGCGGTTACTGAATCA |
| MG64 | 461 | MG64-61 | nucleotide | artificial | | ATTCAATATTTGAGCTTTTTGCAAAAATAAACAGATAT |
| putative | | putative | | sequence | | CAAAGTTAGTTTGCTCGAGCGAAATTAATTGTTATTAG |
| transposon | | transposon | | | | CTAAATATAATTTTTCCCCTCATCAAGAGATGCGATCA |
| end | | end LE | | | | CTACCTTAGTAACTTGCTTGCTAACCTCTGATAAATAA |
| | | | | | | CATCCAACCATACAGGAATCACAGCTTTACCTTCATCC |
| | | | | | | AGGGATGCGATCGCGAATCGCTCCGCAGGAATCGCTC |
| | | | | | | TTTTCCTGTCAACTTATACTAACTAATGTATGATCTAA |
| | | | | | | ATTACTCTTCACTGTCCTTTAGTAATCAGTGAATCTAC |
| | | | | | | CTGTCAACGTGTACGTTCGCACATTATATGTCGTATTT |
| | | | | | | CGCAAGTCATGTCGCAACTGCTTTTAAGGACTAAAACC |
| | | | | | | TTTATTCTATAAGAATCATAAGCTATTTACCCCTACAA |
| | | | | | | AAGACAAATTTATATAATTCGCATATTATATGTCGCAA |
| | | | | | | AACTTGATTTCGCAAATTAAACGTCGTTTATTAAGATT |
| | | | | | | TTGGTATTTTGCAAATTAGATGTCGCATTTTTGAGGAA |
| | | | | | | TTCATGGTACATTAGTACCTTAATTATTCATGAGTGGG |
| | | | | | | TTTCACT |
| MG64 | 462 | MG64-61 | nucleotide | artificial | | TGCAACAAACTTTTTTGTTAGTCATATAATGGGAGGAT |
| putative | | putative | | sequence | | TGAAAGGAGCAACTGATTCGTAATCAGGAATTAAAGT |
| transposon | | transposon | | | | AAAATGTTTATGCAACCCGGATTATTAAGTATGAGCAT |
| end | | end RE | | | | CTAACGAATAATGATAAATACCGTAGTGCAATATTAT |
| | | | | | | GCTACACGAAACTTAATCAGTGTTTACATAATTGGCGA |
| | | | | | | CATTAATTTGCGAAATTATATTAACTATCTATTTAATC |
| | | | | | | AATAAAATGTTGATGAGCGACATTAATTTGCGAAGAA |
| | | | | | | CGACATTAATTTGCGAAATGCGACATATAATTTGCGAA |
| | | | | | | TGTACATACTAAAGCCGATGATGGGATTTGAACCCAC |
| | | | | | | GACCTACTGATTACGAATCAGTTGCTCTACCCCTGAGC |
| | | | | | | CACATCGGCATACACAGCTTAACATTATAGCATTATTT |
| | | | | | | ACCT |
| MG64 | 463 | MG64-57 | nucleotide | artificial | | GTTTAGCTTATTGGGGGAATACTAAGAGGATGTCTGTC |
| putative | | putative | | sequence | | TCAAAAGTGTCATTTTGTCATTCTCAGTAGAGCGAAGT |
| transposon | | transposon | | | | GAGACTAGTACTGTTTTGAGGGAAACGTCGGTGGAAG |
| end | | end LE | | | | CAACTGGTGAGTCCAGTCCTGCACCGACGTTTTCCGCA |
| | | | | | | TCTTGGGATTTAACTTTCCCGTTGGGCGGAACGAGGAG |
| | | | | | | TTCCTTTTTACCGTCAAACTTTGAGATACTGATACGTG |
| | | | | | | TGTTGCATTCATACTTGTCACCTTACCCTGCCCTCACG |
| | | | | | | GGCACTTTTCTCCAAGGCATGCCAAGGGGGCACTTGGT |
| | | | | | | GAGATGATTTCGCCGCAACTTGGTATGAGCGTCCAAG |
| | | | | | | GTGCACACTACCCGTTCATGTACATTCACAAATTAAAT |
| | | | | | | GTCGCTATTTCACAAATTAATGTCGTTTTTTTATTCATA |
| | | | | | | AAACCCTTACCATGCAAGGGTTTTATTTTTAAGGTGAT |
| | | | | | | TTTGGCTACTCTTTTATAATTTAACATATTATAAGTCGT |
| | | | | | | TAACACTATTTTTTCACTAATTAAACGTCGCTTATTTGG |
| | | | | | | ATAAGCGGAAAATACACTTAATTATTTTTCATAAATTA |
| | | | | | | AATGTCGTAAAGTGAGACGTTTGATAGTACATTAGTTT |
| | | | | | | TATTAAGAGCGAGAAATCCTCCACTTATTGCTGTGAGG |
| | | | | | | TGAAA |
| MG64 | 464 | MG64-57 | nucleotide | artificial | | GCTTGTTGCTCTGCAAAGAACCTTCTGTCTTTCTGTTCC |
| putative | | putative | | sequence | | TATACCCAGCCGTGTCAAAAAATCTTGCCTGAAAACTG |
| transposon | | transposon | | | | CACCAGATATTTTATATCTAGCCCATAATTTCAAGGTA |
| end | | end RE | | | | GCTTTGGTATATCACTAGTTTATCAATATCGTCAGTGA |
| | | | | | | AAAGAATCACAAATTTGTTTGATTTTCATTACACCCAC |
| | | | | | | AGCGCCTGAAAGTATGTTTATAGGTTGCTAAATCGGCA |
| | | | | | | GTATGATCAAAATGAGATATTGAGAGCGCGATTTGGA |
| | | | | | | CAAATATAACTCTTCACACTAGTATATTGTCAAATCAG |
| | | | | | | CTTGCTTATGGTACAATGACAAATTAGTGTGATGTTCA |
| | | | | | | AAAAAGCCGAATATGCCAGCATAGCACAGTGGTAGTG |
| | | | | | | CATCCGACTTGTAATCGGAAGGTCGTCGGTTCAAATCC |
| | | | | | | GACTGCTGGCTTTTTACTTCCCATATAGCGCTGTGTTGT |
| | | | | | | ACAGGTGTACATTCACAAATTAAATGTCGCTGTTCACA |
| | | | | | | AATTAACGTCGCATTTCACAAATTGATGTCGTATTTCA |
| | | | | | | CAAATTAACGTCGCATATGCATTCTCTGTAGCCGTTAA |
| | | | | | | CAAATTAAATGTCGCTTACTTTGATTTTGATTTAGCTGT |
| | | | | | | ACAAGGATTGCTATAGCAAAGTTGCTACAAGCAAAGT |
| | | | | | | TAGTACGTAGCCTAATATTTTAACTTTACATAAGCTCT |
| | | | | | | TCCAAACAAAGCAGACGGATTTGAACCC |
| MG64 | 465 | MG64-58 | nucleotide | artificial | | ATGTATTGTCAGAATTTGGTGTTGTACATTAACTAATT |
| putative | | putative | | sequence | | ATTTGTCAATTTAACAAATTATTGTCACTACTCTATCA |
| transposon | | transposon | | | | AATGTTGAAAGCCCTGTCATGGAAAGGTTTCTGACGG |
| end | | end LE | | | | GGTTTTTATTATTTTCACATTCTTAACCCTTCAAAGCTT |
| | | | | | | GATTAACATATTAGCTGTCAAATGCCAGAATTATAACA |
| | | | | | | CATTAATTGTCATTTTCCAAAAAACAAACAAACCAATG |
| | | | | | | ATTTTGCAAAATTTAACAAATTCTTTGTCCAGATGTCA |
| | | | | | | GTATAATACAACTATGTTTTCATAAAACACAAA |
| MG64 | 466 | MG64-58 | nucleotide | artificial | | TTTTGCTGGCAGTGAGATACTTTCAATATTTTGATGTTT |
| putative | | putative | | sequence | | TAAGCCTTAGCTAAAACTAAATGGATTAATCAAGAGT |
| transposon | | transposon | | | | GGAAAGAATAATTTAAAATTATTAGTGGTGGGTTGAA |
| end | | end RE | | | | AGCGAATCCAGTTACGTCCTTATTAACTGGTTAGTGAA |
| | | | | | | CTTAGAAAGCAAACAAATCAATTTCAGTTAAAATCAA |
| | | | | | | GAGGCAAATTTTAGTGACTAGCTTTAGGACACTAATCT |
| | | | | | | GTCAAAAGTGACATTAATTTGTCAACGCGGTCAAATA |
| | | | | | | ATTTGGCAATCGACAACACTAAATTATTTAGCCAACAA |
| | | | | | | AGGTGTCATCGGTATAAATTTATGGACGTAACTGGATT |
| | | | | | | CGAACCAGTGACCCCATCCATGTCAAGGATGTACTCTA |
| | | | | | | ACCAACTGAGCTATACGTCCGAATTTTTCGCAACTTGT |
| | | | | | | TAAGTATAGCATAGCTTATTCTACAAGGCAAGATATAT |
| MG64 | 467 | MG64-59 | nucleotide | artificial | | AAATCTACCCCAAAGCTTTTAAGGGACTGTTTTTTATA |
| putative | | putative | | sequence | | GATGTCACCCAATTTCCAACGGACGAACGTGCAATAC |
| transposon | | transposon | | | | TCAACTGAGCAGCTTTTCTCAAATTTTAAATGGTAGCT |
| end | | end LE | | | | AAAACTGTGACTAAATTTGTGACTAAAATCGTGGGTTG |
| | | | | | | ATTCTACCGGATTTGAACTAAAATGGCACAAACGAAG |
| | | | | | | AATCCACTCCTCGAAGCTAGAAGGTGCTTGTGGAGCG |
| | | | | | | GGAAACAACGGAATATAAACTGTACATTAACTAATTA |
| | | | | | | TTTGTCAATTTAACAAAATAATTGTCATATTATTCAAA |
| | | | | | | ATCGCTCAACCCCTGTCATTGCAATAACTATGGCAGGG |
| | | | | | | ATTTTGCTATTAACAGCAGTCAGACTGCTTTCAACTTT |
| | | | | | | GATTCACAAATTAGATGTCAAAATCCAGATTTTTCACA |
| | | | | | | ATTTAATTGTCAAATCCCAAAAATAGTGGAAAATCAT |
| | | | | | | GATTTTGCATAAATTAACAAATTAGTTGTCACTCAGAT |
| | | | | | | AGTATAATACAACTATGTTTTGATAAAACAC |
| MG64 | 468 | MG64-59 | nucleotide | artificial | | CTTTGCTCCATATGTATAGCGAGATTAGAACTTTATTT |
| putative | | putative | | sequence | | ATGGTGGGTTGAGAGGTAAGGCAGAGGTCTGCCATTA |
| transposon | | transposon | | | | TTGGTGTAAGGACAAGAATCCTTAGTTATGAAAAGCC |
| end | | end RE | | | | ATATAGGTTTTCTAAAAGAATGCGGACATTAATTTGTC |
| | | | | | | AAAAGCAACTTCTACCATTAAAAGTTAAATATCAAGG |
| | | | | | | ACACATAATCTGTTAACAGTGACATCAATTCGTTAAAA |
| | | | | | | GTGACACCAATCTGTTAACAGTGACAAATAATGCGTG |
| | | | | | | AATGTACAATAAACAAAGGCGACACCCGGATTTGAAC |
| | | | | | | CGGGGGATGGAGGTTTTGCAGACCTCTGCCTTACCACT |
| | | | | | | TGGCTATGTCGCCGCACTATATATTTATTATTTTACCA |
| | | | | | | ATATTTGATATAAAAATCTACCCCTCCTAATTTATTTG |
| | | | | | | ATTAAATATCCTTAAAGCATTGAGCAGCCAGAGAAAA |
| | | | | | | GGCCATCGAAGGAAAAACATAGCTGCTAAATATTGAA |
| | | | | | | TACTTCACGAAGAAAACTATGTTTTCTCATAACAGTCT |
| | | | | | | AAAAAGGAGTATGACTCTACAATTGCCATTCCAACTTA |
| | | | | | | CGGTTTATCCATAAATAGGAAGCGAGGACATCTAAAA |
| | | | | | | CTCATCCCGACAATTTTTTCGTTTTCCTTCTACCCATTG |
| | | | | | | CATTGCCTCACATTTGTGCTAGGATTTTCGGTTACGTA |
| | | | | | | GAATACATCTTTTTTTTCATAAAAACAGTATTTATACTT |
| | | | | | | TTTAAGATATAAGTACTGCGAAACATGAAAACCAAAT |
| | | | | | | TTTTATATCTTAGGATAGATGAAAAACATAAAAAAAA |
| | | | | | | ATAAAATTAGTATAAGATATGGAAGCTAATTCTCGGTT |
| | | | | | | TATACTTAAAAAATTGAGAAATAAGTAAACATACATA |
| | | | | | | ACCTTAAAAATATCGAAGACTCAACAGTATTAATATG |
| | | | | | | CTACTAAAGAAGCATTAACTACCAGTATACAAAAGGC |
| | | | | | | GTTATGACTACAAAGGAGAATTGGATATTAATACTCGT |
| | | | | | | ATTAGATTGGTATCACCAAAGA |
| E coli | 469 | pJ23119 | nucleotide | artificial | | TTGACAGCTAGCTCAGTCCTAGGTATAATGCTAGC |
| promoter | | | | sequence | | |
| Linker | 470 | MCV IRES | nucleotide | | | CGTTACTGGCCGAAGCCGCTTGGAATAAGGCCGGTGT |
| | | | | | | GCGTTTGTCTATATGTTATTTTCCACCATATTGCCGTCT |
| | | | | | | TTTGGCAATGTGAGGGCCCGGAAACCTGGCCCTGTCTT |
| | | | | | | CTTGACGAGCATTCCTAGGGGTCTTTCCCCTCTCGCCA |
| | | | | | | AAGGAATGCAAGGTCTGTTGAATGTCGTGAAGGAAGC |
| | | | | | | AGTTCCTCTGGAAGCTTCTTGAAGACAAACAACGTCTG |
| | | | | | | TAGCGACCCTTTGCAGGCAGCGGAACCCCCCACCTGG |
| | | | | | | CGACAGGTGCCTCTGCGGCCAAAAGCCACGTGTATAA |
| | | | | | | GATACACCTGCAAAGGCGGCACAACCCCAGTGCCACG |
| | | | | | | TTGTGAGTTGGATAGTTGTGGAAAGAGTCAAATGGCT |
| | | | | | | CCCCTCAAGCGTATTCAACAAGGGGCTGAAGGATGCC |
| | | | | | | CAGAAGGTACCCCATTGTATGGGATCTGATCTGGGGC |
| | | | | | | CTCGGTGCACATGCTTTTCATGTGTTTAGTCGAGGTTA |
| | | | | | | AAAAACGTCTAGGCCCCCCGAACCACGGGGACGTGGT |
| | | | | | | TTTCCTTTGAAAAACACGATGATAATA |
| Nuclear | 471 | Nucleoplasm | protein | | | KRPAATKKAGQAKKKK |
| Localizing | | in NLS | | | | |
| Signal | | | | | | |
| Nuclear | 472 | SV40 2x | protein | | | PKKKRKVDGSPKKKRKVDS |
| Localizing | | NLS | | | | |
| Signal | | | | | | |
| MG64 active | 473 | MG64-2 | nucleotide | artificial | | GAATTAATAGCGCCGCCGTTCATGCTTCTAGGAGCCTC |
| effectors | | active | | sequence | | TGAAAGGTGACAAATGCGGGTTAGTTTGGCTGTTGTCA |
| sgRNA | | effector split | | | | GACAGTCTTGCTTTCTGACCCTGGTAGCTGCCCACCCC |
| | | sgRNA 1-1 | | | | GAAGCTGCTGTTCCTTGTGAACAGGAATTAGGTGCGCC |
| | | | | | | CCCAGTAATAAGGGTATGGGT |
| MG64 active | 474 | MG64-2 | nucleotide | artificial | | GTTACCACAGTGGTGGCTACTGAATCACCTCCGAGCA |
| effectors | | active | | sequence | | AGGAGGAACCCACTGAAAGGTGGGTTGAAAG |
| sgRNA | | effector split | | | | |
| | | sgRNA 1-2 | | | | |
| MG64 active | 475 | MG64-2 | nucleotide | artificial | | GAATTAATAGCGCCGCCGTTCATGCTTCTAGGAGCCTC |
| effectors | | active | | sequence | | TGAAAGGTGACAAATGCGGGTTAGTTTGGCTGTTGTCA |
| sgRNA | | effector split | | | | GACAGTCTTGCTTTCTGACCCTGGTAGCTGCCCACCCC |
| | | sgRNA 2-1 | | | | GAAGCTGCTGTTCCTTGTGAACAGGAATTAGGTGCGCC |
| | | | | | | CCCAGTAATAAGGGTATGGGTTTACCAC |
| MG64 active | 476 | MG64-2 | nucleotide | artificial | | GAGTGGTGGCTACTGAATCACCTCCGAGCAAGGAGGA |
| effectors | | active | | sequence | | ACCCACTGAAAGGTGGGTTGAAAG |
| sgRNA | | effector split | | | | |
| | | sgRNA 2-2 | | | | |
| MG64 active | 477 | MG64-2 | nucleotide | artificial | | GAATTAATAGCGCCGCCGTTCATGCTTCTAGGAGCCTC |
| effectors | | active | | sequence | | TGAAAGGTGACAAATGCGGGTTAGTTTGGCTGTTGTCA |
| sgRNA | | effector split | | | | GACAGTCTTGCTTTCTGACCCTGGTAGC |
| | | sgRNA 3-1 | | | | |
| MG64 active | 478 | MG64-2 | nucleotide | artificial | | GTGCCCACCCCGAAGCTGCTGTTCCTTGTGAACAGGAA |
| effectors | | active | | sequence | | TTAGGTGCGCCCCCAGTAATAAGGGTATGGGTTTACCA |
| sgRNA | | effector split | | | | CAGTGGTGGCTACTGAATCACCTCCGAGCAAGGAGGA |
| | | sgRNA 3-2 | | | | ACCCACTGAAAGGTGGGTTGAAAG |
| MG64 active | 479 | MG64-2 | nucleotide | artificial | | GAATTAATAGCGCCGCCGTTCATGCTTCTAGGAGCCTC |
| effectors | | active | | sequence | | TGAAAGGTGACAAATGCGGGTTAGTTTGGCTGTTGTCA |
| sgRNA | | effector split | | | | GACAGTCTTGCTTTCTGACCCTGGTAGCTGCCCACCCC |
| | | sgRNA 4-1 | | | | G |
| MG64 active | 480 | MG64-2 | nucleotide | artificial | | GAAGCTGCTGTTCCTTGTGAACAGGAATTAGGTGCGCC |
| effectors | | active | | sequence | | CCCAGTAATAAGGGTATGGGTTTACCACAGTGGTGGC |
| sgRNA | | effector split | | | | TACTGAATCACCTCCGAGCAAGGAGGAACCCACTGAA |
| | | sgRNA 4-2 | | | | AGGTGGGTTGAAAG |
| MG64 active | 481 | MG64-2 | nucleotide | artificial | | GAATTAATAGCGCCGCCGTTCATGCTTCTAGGAGCCTC |
| effectors | | active | | sequence | | TGAAAGGTGACAAATGCGGGTTAGTTTGGCTGTTGTCA |
| sgRNA | | effector split | | | | GACAGTCTTGCTTTCTGACCCTGGTAGCTGCCCACCCC |
| | | sgRNA 5-1 | | | | GAAGCTGCTGTTCCT |
| MG64 active | 482 | MG64-2 | nucleotide | artificial | | GTGTGAACAGGAATTAGGTGCGCCCCCAGTAATAAGG |
| effectors | | active | | sequence | | GTATGGGTTTACCACAGTGGTGGCTACTGAATCACCTC |
| sgRNA | | effector split | | | | CGAGCAAGGAGGAACCCACTGAAAGGTGGGTTGAAA |
| | | sgRNA 5-2 | | | | G |
| MG64 active | 483 | MG64-2 | nucleotide | artificial | | AATTAATAGCGCCGCCGTTCATGCTTCTAGGAGCCTCT |
| effectors | | active | | sequence | | GAAAGGTGACAAATGCGGGTTAGTTTGGCTGTTGTCA |
| sgRNA | | effector | | | | GACAGTCTTGCTTTCTGACCCTGGTAGCTGCCCACCCC |
| | | extended | | | | GAAGCTGCTGTTCGGTCGG |
| | | split sgRNA | | | | |
| | | 5-1 | | | | |
| MG64 active | 484 | MG64-2 | nucleotide | artificial | | CCGACCGAACAGGAATTAGGTGCGCCCCCAGTAATAA |
| effectors | | active | | sequence | | GGGTATGGGTTTACCACAGTGGTGGCTACTGAATCACC |
| sgRNA | | effector | | | | TCCGAGCAAGGAGGAACCCACTGAAAGGTGGGTTGAA |
| | | extended | | | | AG(N23) |
| | | split sgRNA | | | | |
| | | 5-2 | | | | |
| MG64 active | 485 | MG64-2 | nucleotide | artificial | | GAATTAATAGCGCCGCCGTTCATGCTTCTAGGAGCCTC |
| effectors | | sgRNA | | sequence | | TGAAAGGTGACAAATGCGGGTTAGTTTGGCTGTTGTCA |
| sgRNA | | truncation 1 | | | | GACAGTCTTGCTTTCTGACCCTGGTAGCTGCCCACCCC |
| | | | | | | GAAGCTGCTGTTCCTTGTGAACAGGAATTAGGTGCGCC |
| | | | | | | CCCAGTAATAAGGGTATGGGTTTACCACAGTGGTGGC |
| | | | | | | TACTGAATCAGAAAG |
| MG64 active | 486 | MG64-2 | nucleotide | artificial | | GAATTAATAGCGCCGCCGTTCATGCTTCTAGGAGCCTC |
| effectors | | sgRNA | | sequence | | TGAAAGGTGACAAATGCGGGTTAGTTTGGCTGTTGTCA |
| sgRNA | | truncation 2 | | | | GACAGTCTTGCTTTCTGACCCTGGTAGCTGCCCACCCG |
| | | | | | | AAAGGGTATGGGTTTACCACAGTGGTGGCTACTGAAT |
| | | | | | | CACCTCCGAGCAAGGAGGAACCCACTGAAAGGTGGGT |
| | | | | | | TGAAAG |
| MG64 active | 487 | MG64-2 | nucleotide | artificial | | GCGCCGCCGTTCAGAAATGAAAGGTGACAAATGCGGG |
| effectors | | sgRNA | | sequence | | TTAGTTTGGCTGTTGTCAGACAGTCTTGCTTTCTGACCC |
| sgRNA | | truncation 3 | | | | TGGTAGCTGCCCACCCCGAAGCTGCTGTTCCTTGTGAA |
| | | | | | | CAGGAATTAGGTGCGCCCCCAGTAATAAGGGTATGGG |
| | | | | | | TTTACCACAGTGGTGGCTACTGAATCACCTCCGAGCAA |
| | | | | | | GGAGGAACCCACTGAAAGGTGGGTTGAAAG |
| MG64 active | 488 | MG64-2 | nucleotide | artificial | | GAATTAATAGCGCCGCCGTTCATGCTTCTAGGAGCCTC |
| effectors | | sgRNA | | sequence | | TGAAAGGTGACAAATGCGGGTTAGTTTGGCTGTTGTCA |
| sgRNA | | truncation 4 | | | | GACAGTCTTGCTTTCTGACCCTGGTAGCTGCCCACCCC |
| | | | | | | GAAGCTGCTGTTCCTTGTGAACAGCAGTAATAAGGGT |
| | | | | | | ATGGGTTTACCACAGTGGTGGCTACTGAATCACCTCCG |
| | | | | | | AGCAAGGAGGAACCCACTGAAAGGTGGGTTGAAAG |
| MG64 active | 489 | MG64-2 | nucleotide | artificial | | GAATTAATAGCGCCGCCGTTCATGCTTCTAGGAGCCTC |
| effectors | | sgRNA | | sequence | | TGAAAGGTGACAAATGCGGGTTAGTTTTGTTAGACAG |
| sgRNA | | truncation 5 | | | | TCTTGCTTTCTGACCCTGGTAGCTGCCCACCCCGAAGC |
| | | | | | | TGCTGTTCCTTGTGAACAGGAATTAGGTGCGCCCCCAG |
| | | | | | | TAATAAGGGTATGGGTTTACCACAGTGGTGGCTACTG |
| | | | | | | AATCACCTCCGAGCAAGGAGGAACCCACTGAAAGGTG |
| | | | | | | GGTTGAAAG |
| MG64 active | 490 | MG64-2 | nucleotide | artificial | | GAATTAATAGCGCCGCCGTTCATGCTTCTAGGAGCCTC |
| effectors | | sgRNA | | sequence | | TGAAAGGTGACAAATGCGGGTTAGTTTGGCTGTTGTCA |
| sgRNA | | truncation 6 | | | | GACAGTCTTGCTTTCTGACCCTGGTAGCTGCCCACCCC |
| | | | | | | GAAGCTGCTGTTCCTTGTGAACAGGAATTAGGTGCGCC |
| | | | | | | CCCAGTAATAAGGGTATGGGTTTACCACAGTGGTGGC |
| | | | | | | TACTGAATCACCTCCGAGCAAGGAGGAGAAAG |
| MG64 | 491 | MG64-57 | nucleotide | artificial | | AATAATAGCGCCGCAGTTCATGTTCTTAGGAACCGCTG |
| effectors | | effector | | sequence | | AACTGTGAAAAATCTGGGTTAGTTTGACTATTGGAAG |
| sgRNA | | sgRNA | | | | ATAGTCTTGCTTTCTGACCCTAGTAGCTGCTCACCCCG |
| | | | | | | ATGCTGCTGTCTGCGGACAGGAATTAGGTGCGCTCCCA |
| | | | | | | GCAAAAAGGGCGCGGATATACTGCTGTAGTGGCTACC |
| | | | | | | AAATCACCCTCGACCAAGGGGGAACCCATCCCGAAAG |
| | | | | | | GGCGGGTTGAAAG(N23) |
| MG64 | 492 | MG64-63 | nucleotide | artificial | | TAATAGCGCAGCCGTTCATGTTGTTTACAGCCTCTGAA |
| effectors | | effector | | sequence | | CTGTAATGGGTTAGTTTGACTGTTGGAAGACAGTCGTG |
| sgRNA | | sgRNA | | | | CTTTCTGACCCTGGTAGCTGCTCACCCCGATGCTGCTG |
| | | | | | | TCTCTTGAGACAGGATAGGTGCGCTCCCAGCAATAAG |
| | | | | | | GGCGCGGATGTACCGCTGTAGTGGCTACCGAACCACC |
| | | | | | | CCCGATCAAGGGGGAACCCGCTTGAAAGGGCAGGTTG |
| | | | | | | AAAG(N23) |
| MG108 | 493 | MG108-2 | nucleotide | artificial | | AATAGCGCCGTAGTTCATGCTTGCTAAAGCCTCTGAAT |
| effectors | | effector | | sequence | | TGCGAAAAGTCCGGGTTAGTGCTGTCGGCAGACAGCG |
| sgRNA | | sgRNA | | | | TTGCTTTCTGACCCTGGTAGCTGCCCACCCCGATGCTG |
| | | | | | | CTGTCCCTTGCAGACAGGAACCAGGTGCGCCCCCAGT |
| | | | | | | AATAAGGGTGTGGGTTTACCACAGTGGTGGCTACTGA |
| | | | | | | ATCACCTCCGAGCAAGGAGGAATCCACCGAAAGGTGG |
| | | | | | | GTTGAAAG(N23) |
| MG190 | 494 | MG190-1 | protein | unknown | uncultivated | MALLQQRKQEIISDYQVHETDTGSSDVQVAMLTERINKL |
| ribosomal | | ribosomal | | | organism | SAHLKGNKKDHASRRGLLKMIGQRKRLLAYILRQDKDR |
| proteins | | protein S15 | | | | YRALITRLGIRG |
| MG190 | 495 | MG190-2 | protein | unknown | uncultivated | MALLQQRKQEIITDYQVHETDTGSSDVQVAMLTERINKL |
| ribosomal | | ribosomal | | | organism | SSHLKGNKKDHASRRGLLKMIGQRKRLLAYIMRQDKDR |
| proteins | | protein S15 | | | | YRALITRLGIRG |
| MG190 | 496 | MG190-3 | protein | unknown | uncultivated | MPLQQERKQTVINDFQTHGTDTGSADVQVALLTARVEQ |
| ribosomal | | ribosomal | | | organism | LSEHLKKNKKDHASRRGLLQIIGRRKRLLAYILKQDRER |
| proteins | | protein S15 | | | | YQALIKKLGIRG |
| MG190 | 497 | MG190-4 | protein | unknown | uncultivated | MPLQQERKQTAINEYQTHSTDTGSAEVQVALLTARVEQL |
| ribosomal | | ribosomal | | | organism | SEHLKKNKKDHSSRRGLLQIIGRRKRLLAYILKNDREHY |
| proteins | | protein S15 | | | | QALIKKLGIRG |
| MG190 | 498 | MG190-5 | protein | unknown | uncultivated | MPLQQERKQTVINDFQTHGTDTGSADVQVALLTARVEQ |
| ribosomal | | ribosomal | | | organism | LSEHLKKNKKDHASRRGLLMIIGRRKRLLAYILKEDRAR |
| proteins | | protein S15 | | | | YQALIKKLGIRG |
| MG190 | 499 | MG190-6 | protein | unknown | uncultivated | MPLKQEIKQKLINEYQAHPTDTGSAELQVAMLTARVQQ |
| ribosomal | | ribosomal | | | organism | LSEHLKANKKDHASRRGLLKIIGRRKRLLAYILKRDRDA |
| proteins | | protein S15 | | | | YQALIQKLGIRG |
| MG190 | 500 | MG190-7 | protein | unknown | uncultivated | MALTQERKQQLITEYQVHETDTGSTNVQIAILTDRINKLS |
| ribosomal | | ribosomal | | | organism | EHLKTNKNDHSSRRGLLKLIGQRKRLLSYISKENKERYQ |
| proteins | | protein S15 | | | | ALIGRLGIRG |
| MG190 | 501 | MG190-8 | protein | unknown | uncultivated | MALTQERKQEIMGQYQAHETDTGSADLQVAMLSDRINK |
| ribosomal | | ribosomal | | | organism | LSAHLKVNQKDFSSRRGLMQLIGRRRRLLSYIQKQDRAR |
| proteins | | protein S15 | | | | YQALIARLGIRG |
| MG190 | 502 | MG190-9 | protein | unknown | uncultivated | MSLTQQRKQEIMTEYQVHETDTGSAEVQVAMMTERINR |
| ribosomal | | ribosomal | | | organism | LSAHLKANHKDHASRRGLLTIIGQRKRLLAYIQKKDQQN |
| proteins | | protein S15 | | | | YQALIGRLGIRG |
| MG190 | 503 | MG190-10 | protein | unknown | uncultivated | MRDCCINLEKSEIESIMALTQQHKQEIISNYQVHETDTGS |
| ribosomal | | ribosomal | | | organism | ADVQIAMLTERINRLSQHLQANKKDHSSRRGLLKLIGQR |
| proteins | | protein S15 | | | | KRLLSYVQQESREKYQALIGRLGIRG |
| MG190 | 504 | MG190-11 | protein | unknown | uncultivated | MALTQERKQEILTQYQVHETDTGSADVQVAMLTARIIRL |
| ribosomal | | ribosomal | | | organism | SEHLQANKKDHSSRRGLLKLIGQRKRLLSYILEENRERYQ |
| proteins | | protein S15 | | | | ALIGRLGIRG |
| MG190 | 505 | MG190-12 | protein | unknown | uncultivated | MALTQQRKQEIITQYQVHETDTGSSDVQVAMLTARIMRL |
| ribosomal | | ribosomal | | | organism | SEHLQGNKKDHSSRRGLLKLIGQRKRLLSYIMQEDRERY |
| proteins | | protein S15 | | | | QALIARLGIRG |
| MG190 | 506 | MG190-13 | protein | unknown | uncultivated | MALTQERKQEIIVNYQVHETDTGSAEVQVAMLTERINRL |
| ribosomal | | ribosomal | | | organism | SLHLQANKKDHSSRRGLLKLIGQRKRLLAYILKDSREKY |
| proteins | | protein S15 | | | | QALIGRLGIRG |
| MG190 | 507 | MG190-14 | protein | unknown | uncultivated | MTLTQQRKQELITQYQVHETDTGSADLQVAMLTERINRL |
| ribosomal | | ribosomal | | | organism | SQHLQANKKDHSSRRGLLKLIGQRKRLLSYIQEGSRERY |
| proteins | | protein S15 | | | | QALIARLGIRG |
| MG190 | 508 | MG190-15 | protein | unknown | uncultivated | MTLTQERKHEIIDGYQVHDTDTGSVDVQVAVLTERINRL |
| ribosomal | | ribosomal | | | organism | SNHLKTNKKDHSSRRGLLQMIGRRKRLLSYLRKEDIARY |
| proteins | | protein S15 | | | | QNLIQRLGIRG |
| MG190 | 509 | MG190-16 | protein | unknown | uncultivated | MTLTQQRKQELITQYQVHETDTGSAEVQVAMLTERINRL |
| ribosomal | | ribosomal | | | organism | SQHLQTNKKDHSSRRGLLKLIGQRKRLLSYIQEGSRERY |
| proteins | | protein S15 | | | | QALIGRLGIRG |
| MG190 | 510 | MG190-17 | protein | unknown | uncultivated | MPLLQQRKQELISDYQVHETDTGSADVQVAMLTERINRL |
| ribosomal | | ribosomal | | | organism | SEHLKTNKKDHASRKGLLRMIGLRKRLLSYIQKQDNARY |
| proteins | | protein S15 | | | | RALITRLGIRG |
| MG190 | 511 | MG190-18 | protein | unknown | uncultivated | MTLTQARKQEIMSAHQLHATDTGSADLQVAMLTERITR |
| ribosomal | | ribosomal | | | organism | LSEHLKANKSDHASRRGLLKMIGQRKRLLAFINTESVQR |
| proteins | | protein S15 | | | | YQSLADSLGIRRVK |
| MG190 | 512 | MG190-19 | protein | unknown | uncultivated | MSLTQERKQEIMSAHQLHATDTGSSDLQVAMLTERINRL |
| ribosomal | | ribosomal | | | organism | SEHLKANKSDHASRRGLLKMIGQRKRLLAFINTESVQRY |
| proteins | | protein S15 | | | | QNLADSLGIRRVK |
| MG190 | 513 | MG190-20 | protein | unknown | uncultivated | MALTQERKQEIMGDYQTHETDTGSPDVQVAMLTDRITK |
| ribosomal | | ribosomal | | | organism | LSAHLKINQKDFASRRGLMMMISRRKRLLAYIQKENVDR |
| proteins | | protein S15 | | | | YKALIARLGIRG |
| MG190 | 514 | MG190-21 | protein | unknown | uncultivated | MALTQERKQEIMGDFQTHETDTGSADVQIAMLSDRISKL |
| ribosomal | | ribosomal | | | organism | SAHLKINQKDFASRRGLMMMISRRKRLLAYLQKENVDR |
| proteins | | protein S15 | | | | YKALIARLGIRG |
| MG190 | 515 | MG190-22 | protein | unknown | uncultivated | MSLVQEDKQKIITDFQKHETDTGSVEVQVAMLTERINRL |
| ribosomal | | ribosomal | | | organism | SGHLKTNKKDHGSRIGLLKMISLRKRLLSYVQKLDFARY |
| proteins | | protein S15 | | | | KTLIGRLGIRG |
| MG190 | 516 | MG190-23 | protein | unknown | uncultivated | MALLQERKQEIISEYQVHETDTGSADVQVAMLTERINRL |
| ribosomal | | ribosomal | | | organism | SAHLKGNKKDHASRRGLLKMIGQRKRLLAYILKQDQER |
| proteins | | protein S15 | | | | YRALVTRLGIRG |
| MG190 | 517 | MG190-24 | protein | unknown | uncultivated | MTLTQEQKQEIINTHQVHATDTGSADVQVAMLTDRISRL |
| ribosomal | | ribosomal | | | organism | SLHLQANKKDYASRQGLLQMIGQRKRLLGYINKRSPEKY |
| proteins | | protein $15 | | | | RALITKLGIRG |
| MG190 | 518 | MG190-25 | protein | unknown | uncultivated | MALLQAQKQEIISGYQTHETDTGSADVQIAILTEKINRLS |
| ribosomal | | ribosomal | | | organism | LHLRTNKKDHASRQGLLRMISKRKSLLAYLISEDQTRYR |
| proteins | | protein S15 | | | | ALVTRLGIRG |
| MG190 | 519 | MG190-26 | protein | unknown | uncultivated | MALLQERKQELISEYQIHETDTGSSEVQVAMLTERINKLS |
| ribosomal | | ribosomal | | | organism | AHLKTNKKDHASRRGLLKMIGQRKRLLAYIQKKSQDEY |
| proteins | | protein S15 | | | | RALITRLGIRG |
| MG190 | 520 | MG190-27 | protein | unknown | uncultivated | MSLLQAQKQEIITNYQTHETDTGSADVQIAILTEKVSRLS |
| ribosomal | | ribosomal | | | organism | LHLRTNKKDHASRQGLLRMISQRKRLLAYVLKHDPERY |
| proteins | | protein S15 | | | | RALVTRLGLRG |
| MG190 | 521 | MG190-28 | protein | unknown | uncultivated | MTLLQERKQEIISDYQVHETDTGSADVQVAILTERINRLS |
| ribosomal | | ribosomal | | | organism | AHLKGNKKDHASRRGLLKMIGQRKRLLAYIMKQDQTRY |
| proteins | | protein S15 | | | | RALIGRLGIRG |
| MG190 | 522 | MG190-29 | protein | unknown | uncultivated | MTLLQERKQEIINDFQKHGTDTGSADVQVAMLTERINKL |
| ribosomal | | ribosomal | | | organism | SAHLRENKKDHASRRGLLKMIGQRKRLLAYIQAADQDR |
| proteins | | protein S15 | | | | YRALITRLGIRG |
| MG190 | 523 | MG190-30 | protein | unknown | uncultivated | MTLLQERKQEIINDFQKHGTDTGSADVQVAMLTERINKL |
| ribosomal | | ribosomal | | | organism | SAHLRENKKDHASRRGLLKMIGQRKRLLAYILKEDQGR |
| proteins | | protein S15 | | | | YRALITRLGIRG |
| MG190 | 524 | MG190-31 | protein | unknown | uncultivated | MTLLQERKQEIINDFQKHGTDTGSADVQVAMLTERINKL |
| ribosomal | | ribosomal | | | organism | SAHLRENKKDHASRRGLLKMIGQRKRLLAYIIKEDQDRY |
| proteins | | protein S15 | | | | RALITRLGIRG |
| MG190 | 525 | MG190-32 | protein | unknown | uncultivated | MTLTQSKKQELITQYQTHETDTGSADLQVAILTERINQLT |
| ribosomal | | ribosomal | | | organism | GHLQANPKDHASRRGLLQMIGRRRGLLKYIQQKDQGRY |
| proteins | | protein S15 | | | | QALIGKLGIRR |
| MG190 | 526 | MG190-33 | protein | unknown | uncultivated | MALTQQRKQELITQYQVHETDTGSSDVQVAMLTARIMR |
| ribosomal | | ribosomal | | | organism | LSEHLQGNKKDHSSRRGLLKLIGQRKRLLSYIMQEDRER |
| proteins | | protein S15 | | | | YQALIARLGIRG |
| MG190 | 527 | MG190-34 | protein | unknown | uncultivated | MTLLQERKQEIINEYQTHETDTGSADVQVAMLTERINKL |
| ribosomal | | ribosomal | | | organism | SAHLRENKKDHASRRGLLKMIGQRKRLLAYILKQDQDR |
| proteins | | protein S15 | | | | YRALITRLGIRG |
| MG190 | 528 | MG190-35 | protein | unknown | uncultivated | MALVQERKQEIIGQYQVHETDTGSADVQVAILTERINKL |
| ribosomal | | ribosomal | | | organism | SLHLRSNKKDHASRTGLLKMIGQRKRLLAYIQKGDVDR |
| proteins | | protein S15 | | | | YRALIGRLGIRG |
| MG190 | 529 | MG190-36 | protein | unknown | uncultivated | MTLLQERKQEIINGFQKHGTDTGSADVQVAMLTERINKL |
| ribosomal | | ribosomal | | | organism | SAHLRENKKDHASRRGLLKMIGQRKRLLAYIIKEDQDRY |
| proteins | | protein S15 | | | | RALITRLGIRG |
| MG190 | 530 | MG190-37 | protein | unknown | uncultivated | MTLLQERKQEIISDYQVHETDTGSADVQVAILTERINRLS |
| ribosomal | | ribosomal | | | organism | AHLKINKKDHASRRGLLKMIGQRKRLLAYILKQDQERYR |
| proteins | | protein S15 | | | | ALIGRLGIRG |
| MG190 | 531 | MG190-38 | protein | unknown | uncultivated | MTLLQERKQEIINDFQKHGTDTGSADVQVAMLTERINKL |
| ribosomal | | ribosomal | | | organism | SAHLRENKKDHASRRGLLKMIGQRKRLLAYILKEDQDR |
| proteins | | protein S15 | | | | YRALITRLGIRG |
| MG190 | 532 | MG190-39 | protein | unknown | uncultivated | MALTQQRKQELISDYQVHETDTGSSEVQIAMLTERINRLS |
| ribosomal | | ribosomal | | | organism | EHLKANQQDHSSRRGLLKIIGQRKQLLAYVQKSNKEKY |
| proteins | | protein S15 | | | | QALIARLGIRG |
| MG190 | 533 | MG190-40 | protein | unknown | uncultivated | MALTQQRKQELITGYQVHETDTGSSEVQIAMLTDRITRL |
| ribosomal | | ribosomal | | | organism | SEHLRANQQDHSSRRGLLKIIGQRKRLLAYVQKQNREKY |
| proteins | | protein S15 | | | | QALIARLGIRG |
| MG190 | 534 | MG190-41 | protein | unknown | uncultivated | MPLLQARKQEIIGEYQVHETDTGSSEVQVAMLTDRIIKLS |
| ribosomal | | ribosomal | | | organism | AHLKTNKKDHASRRGLLKMIGQRKRLLAYINKKDPNNY |
| proteins | | protein S15 | | | | RQLITRLGIRG |
| MG190 | 535 | MG190-42 | protein | unknown | uncultivated | MALLQERKQEVISEYQVHETDTGSADVQVAMLTERINRL |
| ribosomal | | ribosomal | | | organism | SAHLKGNKKDHASRRGLLKMIGQRKRLLAYILKQDQER |
| proteins | | protein S15 | | | | YRALVTRLGIRG |
| MG190 | 536 | MG190-43 | protein | unknown | uncultivated | MTLLQERKQEIISGYQVHETDTGSADVQVAILTERINRLS |
| ribosomal | | ribosomal | | | organism | AHLKINKKDHASRRGLLKMIGQRKRLLAYILKQDQERYR |
| proteins | | protein S15 | | | | ALIGKLGIRG |
| MG190 | 537 | MG190-44 | protein | unknown | uncultivated | MVVTCTVGPQPGPSLRFTPQPMPLTTTKKQELINGHQTH |
| ribosomal | | ribosomal | | | organism | GTDTGSVEVQVAMLSERISQLTGHLQKNKHDFSSRQGLL |
| proteins | | protein S15 | | | | KMIGRRKRLLSYLNGISKERYSALIAKLGIRG |
| MG190 | 538 | MG190-45 | protein | unknown | uncultivated | MALTQQRKQELISGYQVHETDTGSADVQIAMLTDRINRL |
| ribosomal | | ribosomal | | | organism | SQHLQANKKDHSSRRGLLKMIGQRKRLLSYIQQDSREKY |
| proteins | | protein S15 | | | | QALIARLGIRG |
| MG190 | 539 | MG190-46 | protein | unknown | uncultivated | MPLDTTKKQELINSHQTHGTDTGSVEVQVAMLSERVSQL |
| ribosomal | | ribosomal | | | organism | TGHLQQNKHDFSSRQGLLKMIGRRKRLLGYLRAQSEDR |
| proteins | | protein S15 | | | | YAQLIAKLGIRG |
| MG190 | 540 | MG190-47 | protein | unknown | uncultivated | MALVQERKQEIISQYQVHETDTGSADVQVAMLTERINKL |
| ribosomal | | ribosomal | | | organism | SLHLRSNKKDHASRTGLLKMIGQRKRLLAYIQKGDTDRY |
| proteins | | protein S15 | | | | RALITRLGIRG |
| MG190 | 541 | MG190-48 | protein | unknown | uncultivated | MALVQERKQEIITQYQVHETDTGSADVQVAILTERINKLS |
| ribosomal | | ribosomal | | | organism | LHLRSNKKDHASRTGLLKMIGQRKRLLAYIQKGDAAHY |
| proteins | | protein S15 | | | | RELIARLGIRG |
| MG190 | 542 | MG190-49 | protein | unknown | uncultivated | MALLQQRKQEIISDYQVHETDTGSSEVQVAMLTERINKL |
| ribosomal | | ribosomal | | | organism | SLHLRTNKKDHASRMGLLKMIGQRKRLLAYINKRSPEKY |
| proteins | | protein S15 | | | | RALITRLGIRG |
| MG190 | 543 | MG190-50 | protein | unknown | uncultivated | MALTQQRKQELISEYQVHDTDTGSTEVQVAMLTERISRL |
| ribosomal | | ribosomal | | | organism | SEHLRSNQKDHSSRRGLLKLIGQRKRLLSFLQSEDKQKY |
| proteins | | protein S15 | | | | QNLLTRLGIRG |
| MG190 | 544 | MG190-51 | protein | unknown | uncultivated | MALTQQRKQELISEYQVHDTDTGSTEVQIAMLTERINRL |
| ribosomal | | ribosomal | | | organism | SEHLKGNQKDFSSRRGLLKLIGQRKRLLSYLQSENRERY |
| proteins | | protein S15 | | | | QTLISRLSIRG |
| MG190 | 545 | MG190-52 | protein | unknown | uncultivated | MTLTQQRKHELINEHQVHETDTGSADVQIAMLTERINRL |
| ribosomal | | ribosomal | | | organism | SAHLKSNKSDHASRRGLLTIIGRRKRLLAYVQKEDLSRY |
| proteins | | protein S15 | | | | QALIAKLGIRG |
| MG190 | 546 | MG190-53 | protein | unknown | uncultivated | MSLTQERKHEIIEGYQVHETDTGSAEVQIAMLTERINRLS |
| ribosomal | | ribosomal | | | organism | EHLKANSKDHSSRRGLLQLIGRRKRLLAYMRRESAERYP |
| proteins | | protein S15 | | | | ALIQRLGIRG |
| MG190 | 547 | MG190-54 | protein | unknown | uncultivated | MALTQERKQEIIVNYQVHETDTGSADVQIAMLTERINRL |
| ribosomal | | ribosomal | | | organism | SLHLQANKKDHSSRRGLLKLIGQRKRLLAYIQKDSREKY |
| proteins | | protein S15 | | | | QALIGRLGIRG |
| MG190 | 548 | MG190-55 | protein | unknown | uncultivated | MITWEIAGLHELTPSMSLTQERKHELINGYQVHETDTGS |
| ribosomal | | ribosomal | | | organism | ADVQIAMLTERINRLSDHLKNNKKDHSSRRGLLTMIGQR |
| proteins | | protein S15 | | | | KRLLAFLRKENTERYQSLIQRLGIRG |
| MG190 | 549 | MG190-56 | protein | unknown | uncultivated | MSLTQERKHEIIDGYQVHETDTGSADVQIAMLTERINRLS |
| ribosomal | | ribosomal | | | organism | EHLKNNKKDHSSRRGLLQMIGRRKRLLSYLRNENKERY |
| proteins | | protein S15 | | | | QALIQRLGIRG |
| MG190 | 550 | MG190-57 | protein | unknown | uncultivated | MTLTQERKQEILQGYQVHETDTGSADVQIAMLTERINRL |
| ribosomal | | ribosomal | | | organism | SEHLKTNKKDHSSRRGLLKMIGQRKRLLGYLRKENIERY |
| proteins | | protein S15 | | | | QALIQRLGIRG |
| MG190 | 551 | MG190-58 | protein | unknown | uncultivated | MTLTQERKHEIIEGYQVHETDTGSADVQIAMLTERINRLS |
| ribosomal | | ribosomal | | | organism | EHLKSNKKDHSSRRGLLKMIGLRKRLLSYLRKEDTARYQ |
| proteins | | protein S15 | | | | ALIQRLGIRG |
| MG190 | 552 | MG190-59 | protein | unknown | uncultivated | MSLTQERKHEIIEGYQVHETDTGSADVQIAILTERINRLSE |
| ribosomal | | ribosomal | | | organism | HLRANSKDHASRRGLLQLIGRRKRLLAYMRKGDMSHYQ |
| proteins | | protein S15 | | | | ALIQRLGIRG |
| MG190 | 553 | MG190-60 | protein | unknown | uncultivated | MLRVITWETAGLYELTLSMTLTQERKHEIIDGYQVHDTD |
| ribosomal | | ribosomal | | | organism | TGSVDVQVAMLTDRINRLSNHLKTNKKDHSSRRGLLQM |
| proteins | | protein S15 | | | | IGRRKRLLSYLRKEDIQRYQNLIQRLGIRG |
| MG190 | 554 | MG190-61 | protein | unknown | uncultivated | MALTQQRKQELISEYQVHDTDTGSSEVQIAMLTERINRLS |
| ribosomal | | ribosomal | | | organism | EHLRGNQKDYSSRRGLLKLIGQRKCLLSYVQAEDRQKY |
| proteins | | protein S15 | | | | QALIARLGIRG |
| MG190 | 555 | MG190-62 | protein | unknown | uncultivated | MALLQERKQEIITDYQIHETDTGSADIQVAMLTERINKLS |
| ribosomal | | ribosomal | | | organism | AHLRENKKDHSSRRGLLKMIGQRKRLLAYILKHDQERY |
| proteins | | protein S15 | | | | RGLLTRLGIRG |
| MG190 | 556 | MG190-63 | protein | unknown | uncultivated | MSLTQERKHEIIDGFQVHETDTGSAEVQIAMLTERINRLS |
| ribosomal | | ribosomal | | | organism | EHLKINKKDHSSRRGLLQMIGRRKRLLSYIRNENVQRYQ |
| proteins | | protein S15 | | | | ALIQRLGIRG |
| MG190 | 557 | MG190-64 | protein | unknown | uncultivated | MALTQQRKQELISEYQVHETDTGSAEVQIAMLTERINRL |
| ribosomal | | ribosomal | | | organism | SQHLKGNQKDFSSRRGLLKLIGQRKSLLSYVQSEDRQKY |
| proteins | | protein S15 | | | | QALIARLGIRG |
| MG190 | 558 | MG190-65 | protein | unknown | uncultivated | MTLTQERKQEIIEGYQVHETDTGSADVQVAMLTERINRL |
| ribosomal | | ribosomal | | | organism | SMHLRSNKKDHASRRGLLKMIGQRKRLLSYIRNGDTAH |
| proteins | | protein S15 | | | | YQALIQRLGIRG |
| MG190 | 559 | MG190-66 | protein | unknown | uncultivated | MTLTQERKHEIIEGYQVHETDTGSADVQIAMLTERINRLS |
| ribosomal | | ribosomal | | | organism | EHLKSNKKDHSSRRGLLKMIGLRKRLLSYLRKEDAQRY |
| proteins | | protein S15 | | | | QALIQRLGIRG |
| MG190 | 560 | MG190-67 | protein | unknown | uncultivated | MSLTQERKHEIIEGYQVHETDTGSAEVQIAILTERINRLSE |
| ribosomal | | ribosomal | | | organism | HLKANSKDHASRRGLLQLIGRRKRLLSYMRRENAERYP |
| proteins | | protein S15 | | | | ALIQRLGIRG |
| MG190 | 561 | MG190-68 | protein | unknown | uncultivated | MRVITWETAGLYESNLSMTLTQERKHELIDGYQVHDTD |
| ribosomal | | ribosomal | | | organism | TGSVDVQVAMLTERINRLSNHLKTNKKDHSSRRGLLQMI |
| proteins | | protein S15 | | | | GRRKRLLSYLRKEDIARYQNLIQRLGIRG |
| MG190 | 562 | MG190-69 | protein | unknown | uncultivated | MALTQQRKQEIMGDFQTHETDTGSADVQVAMLTDRITK |
| ribosomal | | ribosomal | | | organism | LSAHLKINQKDFASRRGLMMMISRRKRLLAYIQKQNVD |
| proteins | | protein S15 | | | | RYKALIARLGIRG |
| MG190 | 563 | MG190-70 | protein | unknown | uncultivated | MTLTQERKQEILQGYQVHETDTGSADVQIAMLTERINRL |
| ribosomal | | ribosomal | | | organism | SEHLKTNKKDHSSRRGLLKMIGQRKRLLGYLRKENIERY |
| proteins | | protein S15 | | | | QGLIQRLGIRG |
| MG190 | 564 | MG190-71 | protein | unknown | uncultivated | MSLTQERKHELINGYQVHETDTGSADVQIAMLTERINRL |
| ribosomal | | ribosomal | | | organism | SEHLKSNKKDHSSRRGLLQMIGRRKRLLTYLRNENLGRY |
| proteins | | protein S15 | | | | QALIQRLGIRG |
| MG190 | 565 | MG190-72 | protein | unknown | uncultivated | MTLPQQRKHEIMTEYQVHETDTGSTDLQIAMLTERINRL |
| ribosomal | | ribosomal | | | organism | STHLKANKKDHASRRGLLTMIGTRKRLLSYLQQEDRPRY |
| proteins | | protein S15 | | | | QALIGRLGIRG |
| MG190 | 566 | MG190-73 | protein | unknown | uncultivated | MALTQQRKQELITGYQVHETDTGSSEVQIAMLTDRITRL |
| ribosomal | | ribosomal | | | organism | SEHLRANQQDHSSRRGLLKIIGQRKRLLSYVQKQNREKY |
| proteins | | protein S15 | | | | QALIARLGIRG |
| MG190 | 567 | MG190-74 | protein | unknown | uncultivated | MPLLQQRKQELISDYQVHETDTGSADVQVAMLTERINRL |
| ribosomal | | ribosomal | | | organism | SSHLKTNKKDHASRKGLLRMIGLRKRLLSYIQKQDNARY |
| proteins | | protein S15 | | | | RALITRLGVRG |
| MG190 | 568 | MG190-75 | protein | unknown | uncultivated | MSLTQQRKQELMTEYQIHETDTGSADLQIAILTERINRLS |
| ribosomal | | ribosomal | | | organism | AHLKINKKDHASRRGLLQIIGHRKRLLAYIQKGNKERYQ |
| proteins | | protein S15 | | | | ALIARLGIRG |
| MG190 | 569 | MG190-76 | protein | unknown | uncultivated | MTLPQQRKHELMTEYQVHETDTGSTDLQIAMLTERINRL |
| ribosomal | | ribosomal | | | organism | STHLKANKKDHASRRGLLKMIGTRKRLLSYLQKEDKAR |
| proteins | | protein S15 | | | | YQALIGRLGIRG |
| MG190 | 570 | MG190-77 | protein | unknown | uncultivated | MALTQQRKQEIMGDFQTHETDTGSADVQVAMLTDRITK |
| ribosomal | | ribosomal | | | organism | LSAHLKINQKDFASRRGLMMMISRRKRLLAYLQRENVD |
| proteins | | protein S15 | | | | RYKALIARLGIRG |
| MG190 | 571 | MG190-78 | protein | unknown | uncultivated | MSSSKKILSQKSNIIQRHQIHDDDTGSPEVQIAILTAEIKNL |
| ribosomal | | ribosomal | | | organism | TEHLKKNPKDYSSRVGLLRKVGRRARLLRYLSSVSLSRY |
| proteins | | protein S15 | | | | KKTIAANNIKDKLSAGLVASNNSSDDSNNSKDE |
| MG190 | 572 | MG190-79 | protein | unknown | uncultivated | MSLLQERKQELINDYQVHATDTGSPEVQIALLSARISQLS |
| ribosomal | | ribosomal | | | organism | EHLRTHKKDFSSQRGLLRLISQRRQLLLYLRKHHFDRYE |
| proteins | | protein S15 | | | | AVIQRLGIRGLRS |
| MG190 | 573 | MG190-80 | protein | unknown | uncultivated | MPLLQERKQQVIDSYRTHPTDTGSSDVQIALLSDRVLQLT |
| ribosomal | | ribosomal | | | organism | THLKEHPKDFSSRRSLLKIIGQRKRLLAYVRRRNPAHYKE |
| proteins | | protein S15 | | | | LITRLGIRG |
| MG190 | 574 | MG190-81 | protein | unknown | uncultivated | MSLTQERKHEIIEGYQIHETDTGSAHVQVAMLTERINRLS |
| ribosomal | | ribosomal | | | organism | EHLKQNQKDHSSRRGLMKMIGQRKRLLSYLRNEEPDKY |
| proteins | | protein S15 | | | | SALIQRLGLRG |
| MG190 | 575 | MG190-82 | protein | unknown | uncultivated | MPLLQEKKQEILLAYRRHTTDTGSSEVQVALLTGRINQLS |
| ribosomal | | ribosomal | | | organism | EHLKVHPKDFACRRSLLKLIGQRKRLLAYIRREDRQQHK |
| proteins | | protein S15 | | | | DLIEKLGIRG |
| MG190 | 576 | MG190-83 | protein | unknown | uncultivated | MALQHDLKQDIINTYQIHSTDTGSADVQVAILTERIKQLS |
| ribosomal | | ribosomal | | | organism | EHLKTNKKDHASRRGLLKIIGRRKRLLAYINKHDSQRYR |
| proteins | | protein S15 | | | | QLIERLGIRG |
| MG190 | 577 | MG190-84 | protein | unknown | uncultivated | MPLLQEQKQEILSVYQRHSTDTGSSDVQVALLTGRIAQL |
| ribosomal | | ribosomal | | | organism | SNHLKLHPKDFASRRSLLKLIGQRKRLLAYIRREDRNRYR |
| proteins | | protein S15 | | | | ELVQKLGIRG |
| MG190 | 578 | MG190-85 | protein | unknown | uncultivated | MALLQERKHEIIADYQRHETDTGSADVQVAMLTERINRL |
| ribosomal | | ribosomal | | | organism | SEHLKSNKKDHSSRHGLLKMIGLRKRLLSYIQAEDRERY |
| proteins | | protein S15 | | | | KALIGRLGIRG |
| MG190 | 579 | MG190-86 | protein | unknown | uncultivated | MALLQQRKQELISEYQIHETDTGSADLQVAMLSERINRLS |
| ribosomal | | ribosomal | | | organism | LHLRSNKKDHASRMGLMKMIGTRKRLLSYIQKQDEKRY |
| proteins | | protein S15 | | | | RALIAKLGIRG |
| MG190 | 580 | MG190-87 | protein | unknown | uncultivated | MSLNQAAKHSIMENYRVHETDTGSPEVQVAILTEKINRL |
| ribosomal | | ribosomal | | | organism | TQHLKLNKKDYSSQRGLLRMIGQRRRLLSYLQNIDKNRY |
| proteins | | protein S15 | | | | GQLIQRLGIRG |
| MG190 | 581 | MG190-88 | protein | unknown | uncultivated | MFSMSLLQEQKQALINEYQMHATDTGSPEVQIALLSTRI |
| ribosomal | | ribosomal | | | organism | NQLSEHLRTHKKDFSSQRGLLRLISQRRQLLLYLRKHHF |
| proteins | | protein S15 | | | | DRYENVIKRLGIRGLRS |
| MG190 | 582 | MG190-89 | protein | unknown | uncultivated | MALLQQRKQELISEYQIHETDTGSADVQVAMLTERINRL |
| ribosomal | | ribosomal | | | organism | SEHLRGNKKDHSSRMGLLKMIGQRKRLLAYIQKQDRDR |
| proteins | | protein S15 | | | | YKALIGRLGIRG |
| MG190 | 583 | MG190-90 | protein | unknown | uncultivated | MALVQHQKQQIISDYQVHGTDTGSADVQVALLTERINRL |
| ribosomal | | ribosomal | | | organism | SQHLQANKKDHTSRRGLLKMIGRRKQLLAYILRHDEQH |
| proteins | | protein S15 | | | | YRGLIERLGIRG |
| MG190 | 584 | MG190-91 | protein | unknown | uncultivated | MALTQTRKQELITEYQVHETDTGSPDLQVALLTERISQLT |
| ribosomal | | ribosomal | | | organism | SHLQANPKDHASRRGLLKMIGKRRSLLGYINKQEPARYQ |
| proteins | | protein S15 | | | | ALIQRLGIRR |
| MG190 | 585 | MG190-92 | protein | unknown | uncultivated | MALVQQRKQEIITAYQVHETDTGSADVQVAMLTERINRL |
| ribosomal | | ribosomal | | | organism | SEHLKTNKKDHSSRHGLLKMIGLRKRLLAYIQRNDRARY |
| proteins | | protein S15 | | | | RALIERLGIRG |
| MG190 | 586 | MG190-93 | protein | unknown | uncultivated | MALVQEKKQELINSYQIHETDTGSADVQVAMLTERINRL |
| ribosomal | | ribosomal | | | organism | SAHLKTNKKDFSSRRGLLKMIGQRKRLLSYIIKQDQQHY |
| proteins | | protein S15 | | | | RELITRLGIRG |
| MG190 | 587 | MG190-94 | protein | unknown | uncultivated | MSLTQESKQEIIDGYQVHETDTGSADVQIAMLTARISQLS |
| ribosomal | | ribosomal | | | organism | SHLKNNKKDHSSRRGLLKMIGRRKRLMSYLRKQDRERY |
| proteins | | protein S15 | | | | QALIERLGIRG |
| MG190 | 588 | MG190-95 | protein | unknown | uncultivated | MVLVQEQKQNIINEYQIHETDTGSADVQVAMLTERINRL |
| ribosomal | | ribosomal | | | organism | SSHLKTNKKDFSSRRGLLRMIGRRKRLLSYILKQDQARY |
| proteins | | protein S15 | | | | RELITRLGIRG |
| MG190 | 589 | MG190-96 | protein | unknown | uncultivated | MALTQERKQEIIDSYQIHDTDTGSADVQIAMLSDRISRLS |
| ribosomal | | ribosomal | | | organism | THLQANKKDHASRRGLLKMIGQRKRLLSYVREGNPEHY |
| proteins | | protein S15 | | | | QALIKRLGIRG |
| MG190 | 590 | MG190-97 | protein | unknown | uncultivated | MSLTQEQKQEIITEHQVHETDTGSPEIQVAMLTKRINQLS |
| ribosomal | | ribosomal | | | organism | AHLKQNKKDYSSTRGLLKMIGHRKRMLAYIRNKDNDKY |
| proteins | | protein S15 | | | | RALIQRLGIRG |
| MG190 | 591 | MG190-98 | protein | unknown | uncultivated | MSITQERKQELISEYQVHGTDTGSSDVQVAILSDRINSLT |
| ribosomal | | ribosomal | | | organism | QHLKVNKKDHASRLGLLKLIGRRRRLLTYIQKQDYEHY |
| proteins | | protein S15 | | | | QQLIRRLGIRR |
| MG190 | 592 | MG190-99 | protein | unknown | uncultivated | MALLQERKQELISEYQVHETDTGSADVQVAMMTERIDK |
| ribosomal | | ribosomal | | | organism | LSQHLHSNKKDYSSRRGLLKMIGRRKRLLSYIAKKDVNQ |
| proteins | | protein S15 | | | | YRELIGRLGIRR |
| MG190 | 593 | MG190-100 | protein | unknown | uncultivated | MALLQEQKQQIISEYQVHETDTGSADVQVAMLTERINQL |
| ribosomal | | ribosomal | | | organism | SAHLKTNKKDHSSRRGLLKIIGQRKRLLSYILKQDQERYR |
| proteins | | protein S15 | | | | ALIKRLGIRG |
| MG190 | 594 | MG190-101 | protein | unknown | uncultivated | MALLQERKQELISEYQVHETDTGSAEVQVAMLTERINKL |
| ribosomal | | ribosomal | | | organism | SQHLRDNKKDYSSRRGLLKMIGRRKRLLSYIAKKDVERY |
| proteins | | protein S15 | | | | RELIGRLGIRR |
| MG190 | 595 | MG190-102 | protein | unknown | uncultivated | MSLIQEQKQALINEYQIHATDTGSPEVQIALLSARINRLSE |
| ribosomal | | ribosomal | | | organism | HLRTHKKDFSSQRGLLRLISQRKQLLLYLRKHHPDRYEA |
| proteins | | protein S15 | | | | LIQRLGIRGLRA |
| MG190 | 596 | MG190-103 | protein | unknown | uncultivated | MALLQQEKQQIIESYRLHDTDTGSAEVQVALLTSRINQLS |
| ribosomal | | ribosomal | | | organism | QHLQRNPKDFNSRRGLLMMIGRRKRLLNYIAKHSPDRFR |
| proteins | | protein S15 | | | | ELAERLNIRVKK |
| MG190 | 597 | MG190-104 | protein | unknown | uncultivated | MALLQQEKQEIIETYRLHDTDTGSAEVQVALLTSRINQLS |
| ribosomal | | ribosomal | | | organism | QHLQKNPKDFNSRRGLMMMIGRRKRLLNYIAKRSPDRF |
| proteins | | protein S15 | | | | RELAERLNIRVKK |
| MG190 | 598 | MG190-105 | protein | unknown | uncultivated | MALLQKEKQEIIERYRLHDTDTGSADVQVALLTSRINQLS |
| ribosomal | | ribosomal | | | organism | QHLQRNPKDFNSRRGLLMMIGRRKRLLNYIAKHHPERFR |
| proteins | | protein S15 | | | | ELVERLNIRVKK |
| MG190 | 599 | MG190-106 | protein | unknown | uncultivated | MSSSKKILSQKSNIIQQHQIHDDDTGSPEVQIAILTAEIKNL |
| ribosomal | | ribosomal | | | organism | TEHLKKNPKDYSSRVGLLRKVGRRARLLRYLSSVSLSRY |
| proteins | | protein S15 | | | | KKTIAANNIKDKLSAGLVASDNSSDDSNSKDE |
| MG190 | 600 | MG190-107 | protein | unknown | uncultivated | MSSSKKILSQKSNIIQQHQIHDDDTGSPEVQIAILTAEIKNL |
| ribosomal | | ribosomal | | | organism | TEHLKKNPKDYSSRVGLLRKVGRRARLLRYLSSVSLSRY |
| proteins | | protein S15 | | | | KKTIAANNIKDKLSAGLVASDNSSDDSNNNKDE |
| MG190 | 601 | MG190-108 | protein | unknown | uncultivated | MALLQERKQEIISDYQIHETDTGSADVQVAILTERINRLS |
| ribosomal | | ribosomal | | | organism | AHLKENKKDHASRRGLLKMIGQRKRLLAYILKHNPDRY |
| proteins | | protein S15 | | | | RALINRLGIRG |
| MG190 | 602 | MG190-109 | protein | unknown | uncultivated | MALLQERKQEIISDYQVHETDTGSADVQVAILTERINRLS |
| ribosomal | | ribosomal | | | organism | AHLRENKKDHASRRGLLKMIGQRKRLLAYILKQDQERY |
| proteins | | protein S15 | | | | RALIGRLGIRG |
| MG190 | 603 | MG190-110 | protein | unknown | uncultivated | MALTQQRKQEIISQYQVHETDTGSADVQIAMLTERINRLS |
| ribosomal | | ribosomal | | | organism | EHLQVNKKDFSSRRGLLKLIGQRKRLLSYIQKENREHYQ |
| proteins | | protein S15 | | | | ALISRLGIRG |
| MG190 | 604 | MG190-111 | protein | unknown | uncultivated | MPLLQQRKQEIISEYQVHETDTGSAEVQVAMLTERINRLS |
| ribosomal | | ribosomal | | | organism | THLRSNKKDHASRMGLMKMIGARKRLLGYIQKKDEQH |
| proteins | | protein S15 | | | | YRDLIGKLGIRG |
| MG190 | 605 | MG190-112 | protein | unknown | uncultivated | MALLQERKQEIISDYQIHETDTGSADVQVAMLTARINRLS |
| ribosomal | | ribosomal | | | organism | EHLKSNKKDHSSRMGLLKMIGHRKRLLAYIQKQDNDRY |
| proteins | | protein S15 | | | | RALITKLGIRG |
| MG190 | 606 | MG190-113 | protein | unknown | uncultivated | MALTQQRKQEIISQYQVHETDTGSADVQIAMLTERINRLS |
| ribosomal | | ribosomal | | | organism | EHLQANKKDFSSRRGLLKLIGQRKRLLSYIQKENREHYQ |
| proteins | | protein S15 | | | | ALISRLGIRG |
| MG190 | 607 | MG190-114 | protein | unknown | uncultivated | MPLAQERKQELISGYQVHETDTGSPEVQVAILSDRINQLT |
| ribosomal | | ribosomal | | | organism | EHLRAHPKDFSSRRGLLKLIGRRRQLLSYLQKNENDRYR |
| proteins | | protein S15 | | | | ALVERLGLRR |
| MG190 | 608 | MG190-115 | protein | unknown | uncultivated | MALTQQRKQEIISSYQVHETDTGSADVQIAMLTARINRLS |
| ribosomal | | ribosomal | | | organism | EHLQANKKDHSSRRGLLKLIGQRKRLLAYIQQDSREKYQ |
| proteins | | protein S15 | | | | ALIGRLGIRG |
| MG190 | 609 | MG190-116 | protein | unknown | uncultivated | MGLPQQRKQELMLEYQIHETDTGSAEVQVAMLTARINQ |
| ribosomal | | ribosomal | | | organism | LSSHLENNSKDHAGRRGLLKMIGQRKRLLSYILKQDRGR |
| proteins | | protein S15 | | | | YQALIGRLGIRG |
| MG190 | 610 | MG190-117 | protein | unknown | uncultivated | MALTQAEKQAIMADYQVHETDTGSADLQVAMLTKRIN |
| ribosomal | | ribosomal | | | organism | QLTQHLKANKKDHSSRRGLLRMIGRRKRLLAFIEKEDRS |
| proteins | | protein S15 | | | | RYLELIGRLGIRR |
| MG190 | 611 | MG190-118 | protein | unknown | uncultivated | MPLSQARKQELMTEYQIHETDTGSADFQVAVLTERISQL |
| ribosomal | | ribosomal | | | organism | SQHLQKNKKDFASQRGLMQMIGRRKRLLGYIRKQDEER |
| proteins | | protein S15 | | | | YRHLIRRLGIRG |
| MG190 | 612 | MG190-119 | protein | unknown | uncultivated | MALLQEQKQQLISDYQIHETDTGSADVQVAMLTERINRL |
| ribosomal | | ribosomal | | | organism | SAHLKENKKDHASRRGLLKMIGQRKRLLAYILKHDQDR |
| proteins | | protein S15 | | | | YRALIGKLGIRG |
| MG190 | 613 | MG190-120 | protein | unknown | uncultivated | MPLLQARKQELISEYQVHETDTGSAVVQVAMLTERINKL |
| ribosomal | | ribosomal | | | organism | SSHLQSNQKDYSSRRGLLKMIGRRKRLLSYIAKHNVDEY |
| proteins | | protein S15 | | | | RELIGRLGLRR |
| MG190 | 614 | MG190-121 | protein | unknown | uncultivated | MALLQERKQQIISDYQIHETDTGSADVQVAMLTERINRLS |
| ribosomal | | ribosomal | | | organism | AHLKENKKDHASRRGLLKMIGQRKRLLAYIQKHDQDRY |
| proteins | | protein S15 | | | | RALIGKLGIRG |
| MG190 | 615 | MG190-122 | protein | unknown | uncultivated | MPLLQEQKQEILTTYQKHSTDTGSSDVQVALLTGRITQLS |
| ribosomal | | ribosomal | | | organism | NHLKLHPKDFASRRSLLKLIGQRKRLLAYIRREDRNRYR |
| proteins | | protein S15 | | | | ELVQKLGIRG |
| MG190 | 616 | MG190-123 | protein | unknown | uncultivated | MPLLQERKQEVINSFRIHPTDTGSSDVQIALLSDRVVQLT |
| ribosomal | | ribosomal | | | organism | NHLKEHPKDFSSRRSLLKIIGQRKRLLAYVRRRDPAHYQ |
| proteins | | protein S15 | | | | ELITRLGIRG |
| MG190 | 617 | MG190-124 | protein | unknown | uncultivated | MSLLQERKQELINEYQMHATDTGSPEVQIALLTDRINQLS |
| ribosomal | | ribosomal | | | organism | EHLRTHKKDFSSQRGLLRLISQRRQLLLYLRKHHLDRYE |
| proteins | | protein S15 | | | | TLIKRLGIRGLRS |
| MG190 | 618 | MG190-125 | protein | unknown | uncultivated | MSLLQEQKQALINEYQMHATDTGSPEVQIALLTDRINQL |
| ribosomal | | ribosomal | | | organism | SEHLRTHKKDFSSQRGLLRLISQRRQLLLYLRKHHLDRY |
| proteins | | protein S15 | | | | ETLIKRLGIRGLRS |
| MG190 | 619 | MG190-126 | protein | unknown | uncultivated | MPLLQEQKQEILSVYQRHSTDTGSSDVQVALLTGRIAQL |
| ribosomal | | ribosomal | | | organism | SNHLKLHPKDFASRRSLLKLIGQRKRLLAYIRREDRNRHR |
| proteins | | protein S15 | | | | ELVQKLGIRG |
| MG190 | 620 | MG190-127 | protein | unknown | uncultivated | MPLLQEQKKEILSLYQRHSTDTGSPEVQIALLTGRINQLS |
| ribosomal | | ribosomal | | | organism | NHLKLHPKDFDSRRSLLKLIGQRKRLLAYLRREDRSRYQ |
| proteins | | protein S15 | | | | ELVEKLGIRG |
| MG190 | 621 | MG190-128 | protein | unknown | uncultivated | MLSSMGSKHFQSAMPLPTARKQEIMAARQIHPTDTGSPD |
| ribosomal | | ribosomal | | | organism | VQIALLTERINQLSGHLQNNPKDYNSRRGLLMMIGKRKR |
| proteins | | protein S15 | | | | LLSYLAKIDEERYRRLVEELNIRVRK |
| MG190 | 622 | MG190-129 | protein | unknown | uncultivated | MSLIQEQKQALINEYQIHATDTGSPEVQIALLSARINRLSE |
| ribosomal | | ribosomal | | | organism | HLRTHKKDFSSQRGLLRLISQRRQLLLYLRKHHPDRYEA |
| proteins | | protein S15 | | | | LIQRLGIRGLRA |
| MG190 | 623 | MG190-130 | protein | unknown | uncultivated | MTLLQARKQELISDYQVHDTDTGSADVQIAMLTDRINQL |
| ribosomal | | ribosomal | | | organism | SAHLQKNKKDYSSRRGLLKMIGHRKRLMAYLLKQDSER |
| proteins | | protein S15 | | | | YRALIQKLGIRG |
| MG190 | 624 | MG190-131 | protein | unknown | uncultivated | MALLQERKQELISEYQVHETDTGSAEVQVAMLTERINKL |
| ribosomal | | ribosomal | | | organism | SQHLQSNKKDYSSRRGLLKMIGRRKRLLSYIANKDAGKY |
| proteins | | protein S15 | | | | RELIGRLGIRR |
| MG190 | 625 | MG190-132 | protein | unknown | uncultivated | MALTQQKKQEIMTEHQTHETDTGSAEVQVALLSERITSL |
| ribosomal | | ribosomal | | | organism | SAHLKVHKKDYSSTRGLLQIIGRRKRLLSFIRQKNPSGYQ |
| proteins | | protein S15 | | | | DLIKRLGIRG |
| MG190 | 626 | MG190-133 | protein | unknown | uncultivated | MSLTQEQKQQIITEHQVHETDTGSPEVQVAMLTERINQLS |
| ribosomal | | ribosomal | | | organism | AHLKKNKKDYSSTRGLLKMIGHRKRLLAYIRNKDNDKY |
| proteins | | protein S15 | | | | RALIQRLGIRG |
| MG190 | 627 | MG190-134 | protein | unknown | uncultivated | MSLTQERKHEIIDGYQLHETDTGSAEVQVAMLSERISRLT |
| ribosomal | | ribosomal | | | organism | EHLKVNSKDHASRRGLLQIIGRRKRLLAYIRKGDKQRYL |
| proteins | | protein S15 | | | | NLIQRLGIRG |
| MG190 | 628 | MG190-135 | protein | unknown | uncultivated | MSLTQEKKQELITQYQVHETDTGSSEVQVAMLTERINRL |
| ribosomal | | ribosomal | | | organism | SKHLQANKKDHSSRRGLLKMIGQRKRLLSYIQSGDRERY |
| proteins | | protein S15 | | | | KTLIRSLGIRG |
| MG190 | 629 | MG190-136 | protein | unknown | uncultivated | MAVIILETAALIKEKSRVMSLTQERKQELISQYQVHETDT |
| ribosomal | | ribosomal | | | organism | GSSDVQVAMLTDRINRLSKHLQVNKKDHSSRRGLLKMI |
| proteins | | protein S15 | | | | GQRKRLLSYIQKGDRERYKTLIRSLGIRG |
| MG190 | 630 | MG190-137 | protein | unknown | uncultivated | MALTQQRKQEIMSEHQTHETDTGSCEVQVAMLTERISKL |
| ribosomal | | ribosomal | | | organism | SEHLKINKKDHASRRGLLQMISRRKSLLGFLQRLDKSRY |
| proteins | | protein S15 | | | | QALIARLGIRG |
| MG190 | 631 | MG190-138 | protein | unknown | uncultivated | MALTQQRKQEIMGEYQAHETDTGSADLQVAMLSDRINQ |
| ribosomal | | ribosomal | | | organism | LSLHLRANQNDFSSRRGLMQLIGRRRRLLSYIKKQNKER |
| proteins | | protein S15 | | | | YQALIARLGIRG |
| MG190 | 632 | MG190-139 | protein | unknown | uncultivated | MALTQQRKLEIMGEYQTHETDTGSADLQVAMLTDRISK |
| ribosomal | | ribosomal | | | organism | LSAHLKINQKDFASRRGLMLMISRRKRLLSYIQKQSVDR |
| proteins | | protein S15 | | | | YKALIARLGIRG |
| MG190 | 633 | MG190-140 | protein | unknown | uncultivated | MALTQQEKQELMSEYQIHETDTGSADLQVAMLTKRISQL |
| ribosomal | | ribosomal | | | organism | TEHLKINKKDHSSRLGLLKMIGRRKRLLAYIQKGDPQRY |
| proteins | | protein S15 | | | | QSLIARLGIRR |
| MG190 | 634 | MG190-141 | protein | unknown | uncultivated | MSLTQEKKQELISQYQVHETDTGSAQVQVAMLTERINRL |
| ribosomal | | ribosomal | | | organism | SKHLQANKKDHSSRRGLLKMIGQRKRLLSYIQKGDRDR |
| proteins | | protein S15 | | | | YKTLIRSLGIRG |
| MG190 | 635 | MG190-142 | protein | unknown | uncultivated | MALTQQQKQELMTEYQVHETDTGSADLQVAMLTKRIEQ |
| ribosomal | | ribosomal | | | organism | LTQHLKVNKKDHSSRKGLLKMIGRRKRLLAYIQKGDPQ |
| proteins | | protein S15 | | | | RYQTLIGRLGIRR |
| MG190 | 636 | MG190-154 | protein | unknown | uncultivated | MTLLQERKQELIAEYQIHETDTGSVDLQIAMLTERINQLS |
| ribosomal | | ribosomal | | | organism | AHLQKNKKDYSSRRGLLKMIGQRKRLMAYLLKKDTERY |
| proteins | | protein S15 | | | | RNLIQKLGIRG |
| MG190 | 637 | MG190-155 | protein | unknown | uncultivated | MTLLQERKQELISEYQVHETDTGSAEVQVAMLTERINKL |
| ribosomal | | ribosomal | | | organism | SQHLQSNKKDYSSRRGLLKMIGRRKRLLSYIAKKDVNQY |
| proteins | | protein S15 | | | | RELIGRLGIRR |
| MG190 | 638 | MG190-156 | protein | unknown | uncultivated | MSLLQEQKHQIISDYQVHETDTGSADVQVAMLTERINRL |
| ribosomal | | ribosomal | | | organism | SDHLKANKQDHSSRRGLLQMIGRRKRLLAYIRKQDLERY |
| proteins | | protein S15 | | | | QALIKRLGIRG |
| MG190 | 639 | MG190-157 | protein | unknown | uncultivated | MSSKHIQAAKPVIVSKHQIHKTDTGSPEVQVAILTEEITKL |
| ribosomal | | ribosomal | | | organism | TDHLKINPKDHSSRRGLLRKVSRRKKLLNYLLGEDKVRY |
| proteins | | protein S15 | | | | IRTCKKNGIRTNAAVMLTMNHPKKVALAEDEKAE |
| MG190 | 640 | MG190-158 | protein | unknown | uncultivated | MTLLQARKQELISDFQVHETDTGSADLQIAMLTARISQLS |
| ribosomal | | ribosomal | | | organism | EHLQKNKKDYSSRRGLLKMIGQRKRLMGYLQKQDSERY |
| proteins | | protein S15 | | | | RALIQKLGIRG |
| MG190 | 641 | MG190-159 | protein | unknown | uncultivated | MALTQQRKQELISDYQVHETDTGSSEVQIAMLTERINRLS |
| ribosomal | | ribosomal | | | organism | EHLRANQQDHSSRRGLLKLIGQRKQLLAYVQKSNKEKY |
| proteins | | protein S15 | | | | QALIARLGIRG |
| MG190 | 642 | MG190-160 | protein | unknown | uncultivated | MALTQERKQELISSYQVHETDTGSAAVQIAMLTERINRLS |
| ribosomal | | ribosomal | | | organism | EHLKSNKKDHSSRRGLLKIIGQRKRLLSYLQTEDREQYQ |
| proteins | | protein S15 | | | | NLIGRLGIRG |
| MG190 | 643 | MG190-161 | protein | unknown | uncultivated | MSLTQERKHEIIEGYQVHETDTGSAEVQIAILTERINRLSE |
| ribosomal | | ribosomal | | | organism | HLKANSKDHSSRRGLLQLIGRRKRLLAYMRRESAERYPA |
| proteins | | protein S15 | | | | LIQRLGIRG |
| MG190 | 644 | MG190-162 | protein | unknown | uncultivated | MALTQQRKQEIINNFQVHGTDTGSTDVQIAMLTERINRLS |
| ribosomal | | ribosomal | | | organism | EHLQANKKDHSSRRGLLKLIGHRKRLLAYLQQESREKYQ |
| proteins | | protein S15 | | | | ALISRLGIRG |
| MG190 | 645 | MG190-163 | protein | unknown | uncultivated | MGDCYIHLEKLETESIMALTQLRKQEIISNYQVHETDTGS |
| ribosomal | | ribosomal | | | organism | ADVQVAMLTERINRLSEHLQANKKDHSSRRGLLKLIGQR |
| proteins | | protein S15 | | | | KRLLAYISQESREKYQALIARLGIRG |
| MG190 | 646 | MG190-164 | protein | unknown | uncultivated | MALTQQRKQELISGFQVHETDTGSADVQIAMLTDRINRL |
| ribosomal | | ribosomal | | | organism | SQHLQANKKDHSSRRGLLKMIGQRKRLLAYIQQNNREK |
| proteins | | protein S15 | | | | YQALIARLGIRG |
| MG190 | 647 | MG190-165 | protein | unknown | uncultivated | MALNQQRKQEVMTSYQVHETDTGSADVQVALLTERINK |
| ribosomal | | ribosomal | | | organism | LSEHLKANSKDHSSRRGLLKMISLRKRLLAYILKQDQQR |
| proteins | | protein S15 | | | | YRKLIERLGIRG |
| MG190 | 648 | MG190-166 | protein | unknown | uncultivated | MALVQERKQEIITEFQVHETDTGSADVQVAMLTERINKL |
| ribosomal | | ribosomal | | | organism | SLHLRSNKKDHASRTGLLKMIGQRKRLLAYIQKGDKDR |
| proteins | | protein S15 | | | | YRALITRLGIRG |
| MG190 | 649 | MG190-167 | protein | unknown | uncultivated | MALLLERKQELLSSYQTHPTDTGSSQVQVAMLTERVNQ |
| ribosomal | | ribosomal | | | organism | LSSHLKTHPKDFSSRRSLLKMIGQRKRLLAYIKQGSQTDY |
| proteins | | protein S15 | | | | KELIQRLGVRG |
| MG190 | 650 | MG190-168 | protein | unknown | uncultivated | MTLTQERKQEIMSQYQLHATDTGSSALQVAMLTERINRL |
| ribosomal | | ribosomal | | | organism | SEHLKTNKSDHASRRGLLKMIGQRKRLLAFVQAESVQSY |
| proteins | | protein S15 | | | | QNLADSLGIRRVKD |
| MG190 | 651 | MG190-169 | protein | unknown | uncultivated | MALLQQRKQEIITDYQIHETDTGSSEVQVAMLTDRINKLS |
| ribosomal | | ribosomal | | | organism | LHLRTNKKDHASRMGLLKMIGQRKRLLAYINKGSQERY |
| proteins | | protein S15 | | | | RALITRLGIRG |
| MG190 | 652 | MG190-170 | protein | unknown | uncultivated | MALLQERKLEIFAEYQKHPTDTGSSDVQVAMLTERVTQL |
| ribosomal | | ribosomal | | | organism | TVHLKLHPKDFSSRRSLLKIIGQRKRLLAYVRNEDRAHY |
| proteins | | protein S15 | | | | KQLIQSLGVRG |
| MG190 | 653 | MG190-171 | protein | unknown | uncultivated | MPLLQQRKQELISDYQVHETDTGSSDVQVAMLTERINRL |
| ribosomal | | ribosomal | | | organism | SEHLKTNKKDHASRKGLLGMIGLRKRLLSYIQKQDNAR |
| proteins | | protein S15 | | | | YRALITRLGIRG |
| MG190 | 654 | MG190-172 | protein | unknown | uncultivated | MALLQERKLEIFSEYQKHPTDTGSSDVQVALLTERVTQL |
| ribosomal | | ribosomal | | | organism | TAHLKLHPKDFFFFLSLLKIIGQRKRLLAYVRNQDRAHY |
| proteins | | protein S15 | | | | KQLIQSLGVRG |
| MG190 | 655 | MG190-173 | protein | unknown | uncultivated | MALLQQRKHEIIADYQVHEMDTGSSDVQVAMLTERINR |
| ribosomal | | ribosomal | | | organism | LSEHLKVNKKDHASRKGLLGMIGLRKRLLAYIQAQDKA |
| proteins | | protein S15 | | | | RYRALITRLGIRG |
| MG190 | 656 | MG190-174 | protein | unknown | uncultivated | MALLQERKLEIFSEYQKHPTDTGSSDVQVALLTERVTQL |
| ribosomal | | ribosomal | | | organism | TTHLKLHPKDFSSRRSLLKIIGQRKRLLAYVRNQDRAHY |
| proteins | | protein S15 | | | | KQLIQSLGVRG |
| MG190 | 657 | MG190-175 | protein | unknown | uncultivated | MSLVQEDKQKIITDFQKHETDTGSVEVQVAMLTERINRL |
| ribosomal | | ribosomal | | | organism | SGHLKTNKKDHGSRIGLLKMISLRKRLLSYVQKLDYARY |
| proteins | | protein S15 | | | | KTLIGRLGIRG |
| MG190 | 658 | MG190-176 | protein | unknown | uncultivated | MALTQEKKQELIEGFKTHSTDTGSPEVQVAMLTERITQL |
| ribosomal | | ribosomal | | | organism | TQHLRVNPKDFASRRGLLKIISQRKQLLGYVAKMDTPRY |
| proteins | | protein S15 | | | | QKIVERLGLRR |
| MG190 | 659 | MG190-177 | protein | unknown | uncultivated | MALTQQRKQELICNFQVHETDTGSADVQIAMLTERINRL |
| ribosomal | | ribosomal | | | organism | SEHLQANKKDHSSRRGLLKMIGQRKRLLSYIHGENREKY |
| proteins | | protein S15 | | | | QALIGRLGIRG |
| MG64 | 660 | MG64-98 | protein | unknown | uncultivated | MVMKTTRFDVIARKDPKYKKNREKPIDQLEEEALWEVV |
| effectors | | effector | | | organism | QASCHHTPLGIEILKQMEQPSAFPARIEKLKQPQADGILPD |
| | | | | | | IEQEKKWLEAEIKKVCDSLKQQVAFQSLPGRIYSSAVHQS |
| | | | | | | LKPLKGWLEKQWQLLLSISGKKRFLAVVETDADLAQAS |
| | | | | | | DFSWSDIRASAQAILQQTQEEIAAKAEDETAAKDTKQLL |
| | | | | | | NALLKQYEATSDILTCRAIIHLLRNNFKVRRKPENPEKVQ |
| | | | | | | EWLEGKRVEIERLEEQVPRLPRLRNLFPDQAYDEGLEGLI |
| | | | | | | TYPLSGVAVSERIEWLFHYRVLICFFLIYITSVEKSIQLAY |
| | | | | | | CLLHLVRAEVEREEVQFYEWHDDVSDKIDQFLTIPKSLP |
| | | | | | | YPIYFGGDDLRGWQLNQEGRICFKLNGLGDYLFEVRCDR |
| | | | | | | RQLGIVKYFLQDWQTRNKSKKEYSGGLTLLRSAELLVKP |
| | | | | | | KSGKQNAKLPPVDDRQAVVAGYKLSLHCTYDTDYLSRQ |
| | | | | | | GLERVRQRKIAGQLKNLTDKQAKLTKQQAKLQQLEQEM |
| | | | | | | QQEQAGTSPRRRSKRNAQRLEQIEKLKQSISDLQAELERL |
| | | | | | | RPKLERLQQSQLFQRADRPLYEGVANLFVGVCLDLDQH |
| | | | | | | LVVTVVDAMRRKVLTKRTVKQIMGKHYSLLQRYRHLK |
| | | | | | | QEHDKQRQQDQKVGRHNHLSETDLGKQVADAIANGLIA |
| | | | | | | LAQQYKVSTIVLPETKGWRERLYSQLAARAKIKCNGSKK |
| | | | | | | AMARYTKQYGKRLHQWDYNRLSQAIETEAQTVGLTVIF |
| | | | | | | QRPEFRQEAHEQGNQSADEANEQDNQRVNPFELALQIAI |
| | | | | | | AAYDSLQAEDNAEESESADGTLPDSAGDT |
| MG64 | 661 | MG64-99 | protein | unknown | uncultivated | MSQITIQCRLIAPETTRRYLWELASEKNTPLINELIQGVVS |
| effectors | | effector | | | organism | HPDFEIWRQKGRHPTDVVCKHCSQLKAEARFSGQPSRFY |
| | | | | | | MSAEKVVNYIFKSWFKIQNRLQQRLSGKQKWLNILKSDE |
| | | | | | | ELAEICGQPLEKIKKKAIQILKDTKQKYEASQTEEAQSSS |
| | | | | | | QKSFIRSRLFKRYRSAKQPLTQCAIAYLLKNNCKIPKQPE |
| | | | | | | DPQKFSQRRRKSELQAKRLQEQLEARIPKGRDLTGQAWL |
| | | | | | | STLLTAASAVPKDNQEYRRWQDRLLTKPRTIPFPILFETN |
| | | | | | | EDLSWSLNPEGRLCVHENGLREHTLQIYCDQRQLPWFKR |
| | | | | | | FLEDQQTKRANKNKHSSALFTLRSARISWQEIDTKGHPW |
| | | | | | | DNHYLTLSCTVDKRLWSAEGTDEVRQEKAADTAKILTR |
| | | | | | | LNEKDSLSKTQAAYTRRLASTLERLESSFDRPSQPRYQGQ |
| | | | | | | SQIIVGLSLGWDSPLTLAIWKADIQEIVVYRSLQQLLDKD |
| | | | | | | YPLFLKQRREQQKQSHQRHKAQRHGKGNQFGTSNLGQH |
| | | | | | | IDRLLAKAVVKTAKQYGAGSIAIPALDNIRDILQTEIDAR |
| | | | | | | AEQKIPGYLEVQKRYTKQYKSNIHKWSYGRLLDQIISKA |
| | | | | | | NQESLAIEKSKQPLSGTPQAKAKAVAINAYELRKTVHQN |
| MG64 | 662 | MG64-100 | protein | unknown | uncultivated | MSLITIQCRLVADKASLRHLWRLMAEKNTPLINQLLEQL |
| effectors | | effector | | | organism | GQHPNFETWLQKGEVPEDTIKTICNSLKTQERFADQPGRF |
| | | | | | | YTSAVTLVKEAYKSWFALQQQQQRQIKGKERWLKMLK |
| | | | | | | SDIELQQESQCNLNVIRAKATEILDSFFAKFTQDKNKQSK |
| | | | | | | TKKANNTKKNKKISSNTTLFGRLFDTYDKTEDCLSKCAL |
| | | | | | | VYLLKNNCQVSKVDEDPEQYAKNKRKKEIEIERLRNKIK |
| | | | | | | SQNPKGRDITAEKWLGTLEEATKKVPLNEDEAKSWQAS |
| | | | | | | LLKRYNYMPYPIDYESSTDLEWFTNSVDNEKHTGLHNLK |
| | | | | | | NFDSKTKIAIVVFWQIYFLNLALKLKIYSLMKYVYFTMG |
| | | | | | | YYPNKDVNWLNLKNKEGCIFVKFNGLKEKIRNPEFYVCC |
| | | | | | | DSRQLHYFQRFCQDWQILHQDKETYSCGLFILRSARLLW |
| | | | | | | QERKGKGKPWTIHRLILQCSIETRLWTKEETELVRCEKIN |
| | | | | | | KAEKTISKMEQQGNLKKTQVNRLQKELTTRQKLNNPFP |
| | | | | | | GRPSQLLYQGKSNILVGVSFGLDKPATVAVLDATSKKVL |
| | | | | | | TYRSVKQLLGDNYNLLNRQRQQQQRLSHERHKAQKQN |
| | | | | | | APDSGGESELGQYIDRLLADAIVAIAKTYSAGSIVLPKLR |
| | | | | | | DMREIIQSEVQAKAEKKIPGYKEGQQNYAKDYRVSVHR |
| | | | | | | WSYGRLIESIQTQAAKTGISVESGSQPNIGSQQQQARDLA |
| | | | | | | LFAYQERQIEVL |
| MG64 | 663 | MG64-101 | protein | unknown | uncultivated | MSQITIQCRLVASESSRHQLWKFMADLNTPLINELLHQV |
| effectors | | effector | | | organism | NQHPEFETWRQKGKHPNSVVKELCQPLRTDPRFIDQPGR |
| | | | | | | FYDSAIATVNYIYKSWLALMKRLQFQLEGKIRWLGMLK |
| | | | | | | SDAELVEASGVTLESLRAKATEILAQFTLQPDTAEPQSGK |
| | | | | | | EKKRKRTKKSKKLDGESSISDTFQSNTVEEPQPRKEKKR |
| | | | | | | KKTKKSDGERSISDTLFEVYRGTEDNLTRCAISYLLKNGC |
| | | | | | | KISQKDENAEEFANRRRKLEIQIERLIEQLEARTPKGRDLN |
| | | | | | | DAKWLESLLLATHNVPENEAKAKLWQDSLLKKSSKLPF |
| | | | | | | PIGYGSNGDMTWFWQFSLYNVPINLRFFWLWIYIDYLIAI |
| | | | | | | LFLRDALKNEKEWLHNLRINNISLLIKLWDLTVDVNCLA |
| | | | | | | SILFLHESFHNKYKRRICVKFNGLGEHTFKVYCDFRDLH |
| | | | | | | WFYRFLEEQTIKKHYKNKYTTSLFALRTGRLCWQENEG |
| | | | | | | KGKAWNVNRLILYCCVDTRLWTLEGTNEFKKEKAEEIA |
| | | | | | | KSITKTKAKGELNEKQLDSIQRGNTTLANIDNPFPRPSKPL |
| | | | | | | YKGQSHILVGVSFGLENPATVAVVNGSIGKVITYRNIKQL |
| | | | | | | LGNNYRLLNRQRQQKHTLSHKRQIAQKIAAPNQVGESEL |
| | | | | | | GQYVDRLIAQEIVAIAQKYKAGSIVLPKLGDMREQIQSEV |
| | | | | | | QTKAEQKSDLIEVQKKYAKQYRVSVHQWSYGRLIANIQS |
| | | | | | | QAAKAGILIEESKQPIRGSPQEKAKELAIAAYHSREIN |
| MG64 | 664 | MG64-102 | protein | unknown | uncultivated | MPRPPAVPTQVWINPPVTPSPSHRGNTNFSLYSVSFLLTY |
| effectors | | effector | | | organism | SAVRRHLWHLMSEKNTPLVNALLKQVSQQSKFETWQRE |
| | | | | | | STIPRSVIGELCEPLKKIYPDQPQRFYASAILMVTYTYESW |
| | | | | | | LALQQSRRRRLDGKQRWLNVVMSDADLLALSGATLETI |
| | | | | | | QQKAQTILSQLNAEPETQSNSNEKRSKQARRQTNSSNNS |
| | | | | | | SLFARLFEAYEATDDTPTRCAIAYLIKNGGKIPETEEDPEK |
| | | | | | | FARRIHRKQKEVEQLEAQLQARLPKGRDLTGAEFLETLA |
| | | | | | | SATQQLPENVVQAREWQAKLLTRPASLPYPIVFGSSTDV |
| | | | | | | RWGKTAKGRISVSFNGVDKYLKEADSSIQEWFKASKEYP |
| | | | | | | FRLYCDQRQLPFFQRFLQDWQDYQANKDTYPAGLLTLS |
| | | | | | | SAMLAWREGEGKGEPWNVNHLTLYCSFDTRLMTAEGT |
| | | | | | | LAVQQEKATKAQKRLTKPDSDPRIRSTLDRLQNLPKRPS |
| | | | | | | QKPYQSNPEILVGLSIGLASPITAAVINGRTGEVLTYRTLR |
| | | | | | | SLLGDHYQLLNRHRHQQQQNAVQRYRNQKRGVAFQPR |
| | | | | | | ESELGLYVDRLLAKAIIQLAQAYQVGSIIIPNLTHLRELLE |
| | | | | | | SEITAKAEQKCLGSVEAQNQYAKEYRQRIHRWSYNRLIE |
| | | | | | | AIRSQANQLGITVESGFQPIRGNSQEQARDIAIAAYHSRAI |
| | | | | | | AKN |
| MG64 | 665 | MG64-103 | protein | unknown | uncultivated | MSQITIQCRLVASESTRQQLWKLMAGVNSPLINELLEQV |
| effectors | | effector | | | organism | GQHPDFGTWRQKGKLPTGIVEKLCKPLKTDPRFIGQPAR |
| | | | | | | LYKSAIDIVEYIYESRLAQQQRLQHQLEGQTRWLGMLKS |
| | | | | | | DSELVCRLGCSLDTIRTRAAEVLAQATLTNFVKREGEPLL |
| | | | | | | HKSGVESHHSLSKEGSPSLKKTSVTCDPSNSHPSQDKKR |
| | | | | | | KKTKKRNSKNHNRSLSNALFEAYLATEDVLSKCAITYLL |
| | | | | | | KNGCKVSDQEEDIEKFAKRRRKVEIRVERLTEQLASRMP |
| | | | | | | KGRDLKTAKWLETLFVATTTVPQNEAEARTWQASLLKK |
| | | | | | | PKSVPFPIRIKTSDELLWSKNHKGRLCVRFSGFSEHTFEV |
| | | | | | | YCDRRQLHWFQRFLEDQQVKRDSKNQHSSSLFTLRSGQI |
| | | | | | | AWQEDEGEGEPWNVHCLTLYCIVDTRLWTEEGTEQVRQ |
| | | | | | | EKAADIAKVITRMKGKSDLSKTQVGFVKRKHSTLARINN |
| | | | | | | PFPRPSKPLCQGHSHILVGVSLGLEKLATVAVVDASTGK |
| | | | | | | ALTYRSIRQLLGDNYKLLNRASSIQQHNSHERHKAQKRS |
| | | | | | | APNSFGESELGQYVDRLLSGAIIAIAQTHQAGSIVVPKLG |
| | | | | | | EMREIVQSEIQARAEAKCPGCIEAQARYAKQYRCNVHS |
| | | | | | | WSYGRLIQNIQSQAATAGIAVEEEPQPIRGSPQEKARELAI |
| | | | | | | TAYXXXXLTTTLEP |
| MG64 | 666 | MG64-104 | protein | unknown | uncultivated | ALMALWKSLKKEPRFKGLPNCFLKSARLMVHYTYDSWL |
| effectors | | effector | | | organism | ALQKEKQTKLDKIIRWVEVVKSDTELVQISGCSLDTIRAK |
| | | | | | | AQDLLVQINTQNQTQQTKTRRVRKRKSSNSSTANQAKS |
| | | | | | | QFIPEAKQNFNGSAEENNKSTTNQVPEIKYKTFDTLFNTY |
| | | | | | | YMSSDILQKCAIAYLIKNKLKVNNKEEDLKKFTKIVRQK |
| | | | | | | KKKIERLQEQLKSRLPKGRHWIGNEFLDNLEAFGIPESET |
| | | | | | | EWFSLQSALLREHNFLPYPILFGSSDDLSWSKQLKISNNS |
| | | | | | | NLIKENSESEKIRERICVQFKGLKEVIFEISCDRRQLPLFQQ |
| | | | | | | FLKDWTIYSQNTKEHTSSLFLIRSATLIWEDTKKMKNRQ |
| | | | | | | KRQNENKIVNQKSIDSQQEELKKIFQKEIDGEEQPWNRY |
| | | | | | | QLFLHCTVASEFLSKEGTQQLGQKKQELALKAIATLEQKI |
| | | | | | | LELEKEGKSTKNDRESFSRKQGTVRRLNNLDNPFKRPSR |
| | | | | | | PLYQAQPNILLGVSLGSSKLATATVVDVTTEKVLECQGV |
| | | | | | | RCLLGDNYKLLTRKRYLHEMHSHLRSKAQKRGAKNLLR |
| | | | | | | EAELGEHIDRLIATAIIALARKYQASTIVLPNMKDYTEKK |
| | | | | | | QSEIEAFAEQECSGLKFVEKRFTKAQSVKLHQWSYGRLS |
| | | | | | | EIICQQASKVGIAVEIGQQPRHGSSQEQGRAMAIETYHSR |
| | | | | | | KNSLKSKNLRS |
| MG64 | 667 | MG64-105 | protein | unknown | uncultivated | MSVITIQCRLVADDKTLRHLWELMAEKNTPLVNELLDRL |
| effectors | | effector | | | organism | GKHTDFEAWVQAGKVPKTTIKALCDSLKTQEPFIGQPGR |
| | | | | | | FYTSATTLVAYIYKSWLALHKRRQRKIEGKERWLEMLKS |
| | | | | | | DVELEQESNSSLELIRTIATEILSKFSASSTDGRNQKSKGK |
| | | | | | | KSKKVKKDKADEPMSIKPGVLFEAYQKTEDILRRSALVY |
| | | | | | | LIKNNCQVNLAEEDPDKYAKMRRKKEIEIERLKEQLKSR |
| | | | | | | VPKGRDLTGKKWLETLEKAVNSIPQDENEAKSWQAGLL |
| | | | | | | RKSSTVPFPVAYETNEDMHWEISDKGRIFVSFNGLSKLKL |
| | | | | | | EVYCDQRHLPWFQRFVEDQETKRKGKNQHSSGLFTLRS |
| | | | | | | GRLSWLKQEGKAEPWSVNRLILFCSVDTRMWTVEGTQQ |
| | | | | | | VAIEKIADVEQNLTKAKEKGELNSNQQAFVTRQQSTLAK |
| | | | | | | INTPFPRPSKPLYEGKSHILVGVSLGLENPATVAVFDAVN |
| | | | | | | NKVLAYRSVKQLLGNNYNLLNRQQQQKQRLSHDRHKA |
| | | | | | | QKDFTRNDFGESELGQHIDRLLAKEIVAIAVTYFAGSIVL |
| | | | | | | PKLGDMREIIQSEVQARAEKKIPGFKEGQQKYAKEYRKQ |
| | | | | | | VHNWSYGRLIENIQSQAAKVGILIETGQQPIRGSPQEQAR |
| | | | | | | DLALFAYQCRIASSI |
| MG64 | 668 | MG64-106 | protein | unknown | uncultivated | MSVITIQCRLVAPEETLQHLWELMEKKNTPLINELLEQLG |
| effectors | | effector | | | organism | KHPDFETWLQKGKLPTEVVKTLCNSLKTNLCFAGQPGRF |
| | | | | | | YSSAIAFVDYIYKSWFALQKQRQHKIERKERWLSMLKSD |
| | | | | | | GELEQESRCSLDVIRAKAAELLTTVAVQSDSNQNQPTKS |
| | | | | | | NKGSKTKNGKADEVSPTLFNKLFEAYEQTTDTLTRCALA |
| | | | | | | YLLKNGCQVNELEEDSKEFARRRRSKEIEIERLKEQQKSQ |
| | | | | | | IPMGRNLTGIPSLETSEIVIHNASKNQGEAKAGQAVLLRK |
| | | | | | | SNCVPFPINYGSSTDLSWFKNDKGRICVKFNGLGKHSFEI |
| | | | | | | YCDRRQLHWFQRFLEDWQIDHDNKDQYSTSLFALRSAR |
| | | | | | | LLWSEGKGKDDPWNKHHLTLQCSIDTRAWTAEGTELIRS |
| | | | | | | EKIAAADKQLSNQEQKGELNEKQQDYLQRKRSQRERLN |
| | | | | | | NTLPRPSKPLYQGQSSILVGVSFGLDEPATVTVVDATKG |
| | | | | | | KVLAYRNIKQILGKNYPLLTRQRHQQQSLSHKRHKAQK |
| | | | | | | RSAPNQFGESKLGQYVDRLVAKEIVAIAQTYQAGSIVLP |
| | | | | | | KLSDMREIVQSEIQARAEQKVPGYKEGQQKYAKQYRVS |
| | | | | | | VHRWSYGRLSQCIHESAAKVGIVIEIGRQPIRGSPKEKAR |
| | | | | | | DLAIAAYYNRIITQS |
| MG64 | 669 | MG64-107 | protein | unknown | uncultivated | MSFITIQCRLVVGETIRRKLWDLMVNKNTPLVNELLKQV |
| effectors | | effector | | | organism | TQHGDFETWQREGKVSEKPVQDLCKPLRPDPRFENQPGR |
| | | | | | | FYTSANLMVTYTYQSWFALQKKRCRRLDGMRHWLDVV |
| | | | | | | KSDIELVQTSGCDLERLRAKAQAILGQLSAEESSSKARSP |
| | | | | | | KNKKKQNSKADRDLMGRLFLMYETAEDVLSRCAIAHLL |
| | | | | | | KNDCQVNELEENPEKFADRIRNKQKAIEQLEAKLTSRLP |
| | | | | | | KGRDLTGEEFLETLAIATEQIPENEVDQILWQAKLLAKPA |
| | | | | | | TLPYPIVFGSQTDLRWSMNEKSRLCVAFNGVEKFIPELKQ |
| | | | | | | TPFQIYCDQRQLPIFQKFLQDWQAHRANEATYPLSLFLFK |
| | | | | | | TASLGWEQGKGKGDPWQVNRLTLHCTINPDLLTAEGTE |
| | | | | | | QVQQQSIAKFETRLSKVTAQETLTEAQQGSIKRQRSSLAR |
| | | | | | | LQNAPQRPSKPQYQGFPEIMIGVSIGLVCPITVAVINLKTG |
| | | | | | | QALTYRSTRQLLGDNYRLLNRQRQQQQHHTLKRHKNQT |
| | | | | | | KGYIHQPSESELGQYIDRLLANSIIKLAQQFQASCIVLPQT |
| | | | | | | KNLRERLSAEINARAEKKSDSKQVQDKYAKEVRMSIHR |
| | | | | | | WSYNRLLTAISTQAEKTGLAIETIAQPLQGTPQQKAKDV |
| | | | | | | AIAAYHFRQVSSN |
| MG64 | 670 | MG64-108 | protein | unknown | uncultivated | MSFITIQCRLVAPEETLQHLWELMEKKNTPLINELLEQLG |
| effectors | | effector | | | organism | KHPDFETWMQKGKLPTGVVDTLCSSLKTNPCFAGQPGR |
| | | | | | | FYYSASTLVDYIYNSWLALQQKRQRQIEGKERWLSILKS |
| | | | | | | DGELEQESGCSLEVVRAKASQILTQVATQSDSKQNQPPK |
| | | | | | | RKKTKKGNANRPPSTVFNQLFDSYGKTKDSLRRCALTYL |
| | | | | | | LKNDCQVSEVEEDPEKLAHRRRKTEIGIERLKEQLKSRIP |
| | | | | | | KGRDLTGQKWLEALEIANHNVPKDEDEAAAWQAALLR |
| | | | | | | KSSSVPFPINYGSNTYLTWFKNEKGRICVKFNCLGKYPFE |
| | | | | | | IYCDRRQLHWFQRCLEDWQIDHDNKGQYHTGLFTLCSA |
| | | | | | | RLVWLEGKEKGFPWNVYRLTLHCSIDTRAWTAQGTELI |
| | | | | | | RSEKIAAVDKEIRNKEQKGELNEKQQERLQRKYSERQRL |
| | | | | | | NNTFPRPSKPLYQGQPSILVGVSFGLEKPATVSVVDVTKG |
| | | | | | | NVLAYRTVKQLLGDNYKLLTRQRQQQQSSSHQRHKAQ |
| | | | | | | KQSAPNEFGESELGQYVDRLLAKAIVAIAKTYQAGSIILP |
| | | | | | | KLSDMREIVQSEIQARAEQKVPAYKEGQQKYAQQYRVS |
| | | | | | | VHRWSYGRLSQCIHESAAKVGIAIEIGQQAIRGSPQEKAR |
| | | | | | | DLAIAAYHARIATLS |
| MG64 | 671 | MG64-109 | protein | unknown | uncultivated | MSQITIQCQLVASASTRQQLWLLMAQKNTLLINELLQQV |
| effectors | | effector | | | organism | GQHPDFETWRQKGKLQAGIVKALCQPLKTDPRFIGQPAR |
| | | | | | | FYSSAIAVVDYIYRSWLALQKRLQYQLEGQTRWYQMLK |
| | | | | | | SDAELIEICGGSLETLRSKAAEILAQLAPESTSVDPQPTKG |
| | | | | | | KKSNKRKNSSNNPNLSAALFEAYRQTEDILSSCAINYLLK |
| | | | | | | NGCQVSEKEEDPEKFAKRRRSVEIRIERLKEQLASRMPKG |
| | | | | | | RDLTDEKWLETLLVASSTVPNSEFQAKSWQDNLLRKSSL |
| | | | | | | VPFPVAYETNEDMTWFKNSKGRICVKFNGLSEQTFEIYC |
| | | | | | | DFRQLNWFQRFLEDQQIKRNSKSQHSSSLFTLRSGRIAWS |
| | | | | | | EGEGKGDPWNIHRLTLYCSVDTRLWTTEGTEQVRHEKA |
| | | | | | | DEITRIITKTKEKADLNEQQQAFIKRKTSTLARINSSFPRPS |
| | | | | | | KPVYQGHSHILVGVSLGLDKPATLAVIDAIANKVIAYRSI |
| | | | | | | RQLLGDKYQLLNRQRQQQHQNAHKRKIAQRQGIPNQFS |
| | | | | | | ESELGQYIDRLLAKTIVAVAKAYQAGSIVLPKLGDVRESI |
| | | | | | | ESEIKARAEQKCPDLVEVQKQYAKQYRSSIHRWSYARLI |
| | | | | | | DSIKSQASQVGILIEEGKQPVRGSLPEKARELAITAYHSRL |
| | | | | | | NTKS |
| MG64 | 672 | MG64-110 | protein | unknown | uncultivated | MNMPMFTIPCRLCASEETRRDFWQWMEKYTLLVNELLE |
| effectors | | effector | | | organism | KIAEHPQFQEWQKKGDISRKAVREILNPLEKNPCYEGLPR |
| | | | | | | RFYTSAELISCDTYKSWLALQQQRHFQLIGKKRWLQAVE |
| | | | | | | SEFELSAITDFKPDQVCAKAGEIREEALQSLNRQGSKHKS |
| | | | | | | LMGVLLDMHGNTAEAPLNRRAINHLLINELQVTDKEQN |
| | | | | | | LDELSKRLDKKRVEIRRLEEQLTSRLPKGRDPTGQRYLQ |
| | | | | | | MLCHITALPELSDDLEKLEAELDRWSQQQQLPLGKELLY |
| | | | | | | PIRFDSSSDLYWLLKSQETSNPSETDNQIHQELPKSEKPQK |
| | | | | | | KHRQQSKERIHVQFKGTKDYTFKIQCDRRQLPLFRQFLID |
| | | | | | | YQTYKQLPEAERFSEGLFALRSAKLIWRKDDTIHGSNKN |
| | | | | | | RGINKQDDQLKPWNTHRLHLHCTVDRRLLTAEGTEQVR |
| | | | | | | EEKKREVIKKLKGQDQLEESQLQELGLTKNQISDVKRKC |
| | | | | | | STLNRLENYSPSRPSTQPYEGQPHIVMGVSFSRRQPVAIA |
| | | | | | | VVNVETQEVLECQSAKEILNRGEAQYICRHGKKEPLIAD |
| | | | | | | GAERQHPNGGKLYIRKRKRVQGKPYRLVQQLHQRHQH |
| | | | | | | NSRQKVQQQQQNCYREDNSDSNLGVYVDRLIASRIVELA |
| | | | | | | LRRKAGTIVIPQLKGIRESVESDIRAQAERQFPHDKERQK |
| | | | | | | EYAKHYRVSFHGWSYQRLSEFIKECATREGMAVIVRKQP |
| | | | | | | CGGDMEQKAIAIALSP |
| MG64 | 673 | MG64-111 | protein | unknown | uncultivated | MAIFTVQCRLDAWGETPESREAIRRYIWEFMAETYTPLV |
| effectors | | effector | | | organism | NELISEVAEHPAFKTWQQEGSLDSGALMTLWKSLKKEPR |
| | | | | | | FKGLPDRLLVSARLMVHYTYDAWIALQNEKQTKLDKNI |
| | | | | | | RWVEVVKSDAELVQISGCNLDTIRARAKELLAQINTQNQ |
| | | | | | | TQQTTDRRVRKRKASNSSTANQAKSQFIPEAKQNFNGSA |
| | | | | | | EENNKSTTNQVPEIKYKTFDTLFNTYYMSSDILQKCAIAY |
| | | | | | | LIKNKLKVNNKEEDLKKFTKIVRQKKKKIERLQEQLKSR |
| | | | | | | LPKGRHWIGNEFLDNLEAFGIPESETEWFSLQSALLREHN |
| | | | | | | FLPYPILFGSSDDLSWSKQLKISNNSNLIKENSESEKIRERI |
| | | | | | | CVQFKGLKEVIFEISCDRRQLPLFQQFLKDWTIYSQNTKE |
| | | | | | | HTSSLFLIRSATLIWEDTKKMKNRQKRQNENKIVNQKSID |
| | | | | | | TQQEELKKIFQKEIDGEEQPWNRYQLFLHCTVASEFLSKE |
| | | | | | | GTQQLGQKKQELALKAIATLEQKILELEKEGKSTKNDRE |
| | | | | | | SFSRKQGTVRRLNNLDNPFERPSRPLYQAQPNILLGVSLG |
| | | | | | | SSKLATATVVDVTTEKVLECQGVRCLLGDNYKLLTRKR |
| | | | | | | YLHEMHSHLRSKAQKRGAKNLLREAELGEHIDRLIATAII |
| | | | | | | ALARKYQASTIVLPNMKDYTEKKQSEIEAFAEQECSGWK |
| | | | | | | CVEKRFTKAQSVKLHRWSYSRLSKIICQQASKVGIAVEIG |
| | | | | | | QQPRHGSSQEQGRAMAIETYHSRKNSLKSKNLRS |
| MG64 | 674 | MG64-112 | protein | unknown | uncultivated | MNMSMFTIQGRLCASEETRRYFWEKMEKYTLLVNELLE |
| effectors | | effector | | | organism | KIPQQSQFQEWEKKGNISRKIVREILSPLKDNHSYAGLPA |
| | | | | | | RFYTSVELISCETYNSWLALQQKRLFKMLSKKRWLQAV |
| | | | | | | ESEFELLATTDFNPNAVFAKARDIQNEAMHRLNGQGSKQ |
| | | | | | | ESLIGILLDMHDDTAENPLSRRAINHLLINHLKISEEEENL |
| | | | | | | DKLSERLDKKRVEIRRLEEQLNSRLPKGRDPTGQRYLQIL |
| | | | | | | SYITSLPELSNDPQKLEAELDKLTIQQQLPLFNELPYPIRFE |
| | | | | | | SSDDLYWSVLSQDSPNSSHTEHGVHQELPKSEKLQTKHR |
| | | | | | | PQPKERICVQFKGTQNFTFKIQCDHRQLPIFRQFLVDYQT |
| | | | | | | YKKLPETERFSQGLFALRSACLIWRKDDRKHGSKKKRAN |
| | | | | | | DKQEDQLKPWNTHRLYLHCTVDRRLLTAEGTEQVCEEK |
| | | | | | | KNRAIQALKGKQELEVSELQKLGLTKAQISDVERKRSTL |
| | | | | | | NRLENNPYPPRPNTIPYEGQPHIVIGVSFSRNEPVTIVIVDV |
| | | | | | | EKQEVLERQSAEELLNRGEPQYIWRNGKKEPLLRDGTER |
| | | | | | | QHPNGGKLYIRKGKYAQCKPYRLVEQLHRQHQRNCKQR |
| | | | | | | IQEQKQDRYRETNSDSSLGLYVDRLIASRIVSLALQRKAG |
| | | | | | | TIAIPQLKGVRENVESDIRAKAERLFPNEM |
| MG64 | 675 | MG64-113 | protein | unknown | uncultivated | MLTPDLRNFMSIITIPCRLVSREFSRYCLWKLMADQNTP |
| effectors | | effector | | | organism | MINELLALVGNHTEFEAWQQQGKLPAKLVKNLCDSLKS |
| | | | | | | DPRFEGQPSRFYSWAVATVNYIYKSLFAIQQKLRRDLER |
| | | | | | | KSYWLSRLETASSFIQTNKLSWSDLYRKAEEILATAIAQN |
| | | | | | | STDEAQQRNGKKEKKDKTPRKLATILFGLYNQTEDPAEQ |
| | | | | | | CIITCVIANGCCLRKPEEDLPDFTELRREKEIEIERIQEKLK |
| | | | | | | TSRLPKGRDLTGKDWLEALELATFHQLDNEQHQAVQAN |
| | | | | | | LLRESSSLPFPVSYESPPDFTWSRNDKGRICVKLSGLAKH |
| | | | | | | HTFEVYCDRRHLPWFERFLKDQETRHDSEDKLSTSLYTL |
| | | | | | | RSGQLIWKEGEVSKSSSVAPSLFKWLFYYGLQILCSLPEV |
| | | | | | | SLWQSGELKLGLLAYLLLKNRKREPWHVHHLHLHCAVE |
| | | | | | | TLLWTAEGTQQVANEKAARADKTISKMEEQQKKGELNE |
| | | | | | | AQQASLIRTRSMRDRLNNPFPRPSQPLYQGQSNLVVGVS |
| | | | | | | MGLDKPATVAVVDVNTGKVLTYRNVKQLLGKNYKLLN |
| | | | | | | QQRQRQHRDARRRHKAQKKDAPNQFGASKLGQYVDRL |
| | | | | | | LAKAIVAIALTYKASSIALPKLGEVSERVNSQIQARAEKE |
| | | | | | | CLGHKKSYQKYGKQYRSRIHSWSYGRLIENIQQAAAKV |
| | | | | | | GIAIVEGQQPSKGTPQEKAGNVALSAYQNRMRAVS |
| MG64 | 676 | MG64-114 | protein | unknown | uncultivated | MSFVTIQCRLVVSESIRCQLWNLMANKNTPLVSELLKCV |
| effectors | | effector | | | organism | SQHDEFEAWQRSGKVPVKTVRELGEPFKMDSRFEGQPG |
| | | | | | | RFYTSASLMVAYTYKSWLALQRQRRQRLDGKLRWLNIV |
| | | | | | | KSDAQLVQDSGCDLDTIRTKAGEILAHLNAQASPEPPRST |
| | | | | | | KQKRKKKKQQSLAADPRLMNLLFQAYEAMEDTLSRCAI |
| | | | | | | AHLLKNDCQVSAQQEDPEAFTQRIHRKKKEIERLQAQLL |
| | | | | | | SRLPKGRDLTGERFLESLAIAVGKVPDDAIEQLLWQAKL |
| | | | | | | LAKRAAMPYPILFGSQDDLRWSINERGRICVAFNGLDKAI |
| | | | | | | PELKRNPCQIYCDRRQLPLFQRFLADWQTFQANQDTYPL |
| | | | | | | GLFLFQTGLLGWEEGQGKGEPWSVNRLTLHCTLDTHPLT |
| | | | | | | AEGTEQLRQAEIARLTEKLSAVQNPEALTQNQQAWLKR |
| | | | | | | QHSTLARLQNPPQRPSKLVYQGDPEILLGISIGLAHPATA |
| | | | | | | AVVNVRTGKVLEYRTTRQLLGKNYRLLNRQRQQQQLH |
| | | | | | | ALQRHKHQIKGRTSQLGESELGQYVDRLLAKSIVELAHQ |
| | | | | | | FQASCIVLPQTNNLREHLAAEISARAEQKSDSKQAQDKY |
| | | | | | | AKQFRISIHRWSYARLLMTIRHQAEKAGISIETGSQPLRGT |
| | | | | | | SNKKAKELAIATYHLRQVACN |
| MG64 | 677 | MG64-115 | protein | unknown | uncultivated | MTMPMLTIQSLLCASEETRQLFWVWMEKYTFLVNELLE |
| effectors | | effector | | | organism | KIPQHSQFQEWQKKGDIPLKTVREILSPLKGAPQYAGLPG |
| | | | | | | RFYTSAELMSCNTYKSWLALQKERQIRLTGKKRWLHAV |
| | | | | | | ESEFELSAITEFNPDKIRSKAGSLLKKATQKLEKEGGKQK |
| | | | | | | ELIEILLEEHDKTAKNPLSRRAINHLLINDLKISEEEQNLSE |
| | | | | | | LSERLEKKKVEIRRLEEQLTSRLPKGRDPTGQRYLQILCHI |
| | | | | | | SASPELRDDPEKLEAELDKLTEQQQLPLFNTLPYPIRFDSS |
| | | | | | | GDLHWSLENLKSKHWKHPKERICLDFKGVKGRIFKIQCS |
| | | | | | | RRQLPVFRQFLNDYQAHESLLEEERFSEGLFALRSACLIW |
| | | | | | | HKDEMRHRSKKKKQIDQQEDQLKPWNTHRLYLHCTVD |
| | | | | | | RRLLTAEGTEQVREEKKKKTIEALKGKENLEESHLKQLG |
| | | | | | | LNKNQISSVKRQKTTLNRLENYSPPPRPNAKPSEGQSHIV |
| | | | | | | VGVSFSRYEPVTIAIVDVEKKEVLECQSAKELLNRGEAH |
| | | | | | | YIWRNGKKELLKIDGTERQHPNGGKLYIRKGKQVQWKP |
| | | | | | | YRLVKQLHQRHQHNWRQRAKQQQQNRYRQDNSDSNL |
| | | | | | | GLYVDRIISSEIVELALKRKAGTIVIPQLHGIRESIESDIRAQ |
| | | | | | | AERRFPHDKERQKEYLKDYRSSFHRWSYGRLSECIKERA |
| | | | | | | QAESIAVVVQKQPSGGNLEQKAIAMALSSYNVKIS |
| MG64 | 678 | MG64-116 | protein | unknown | uncultivated | MNMSMFTIQGRLCASEETRRYFWEKMEKYTLLVNELLE |
| effectors | | effector | | | organism | KIPQQSQFQEWEKKGNISRKIVREILSPLKDNHSYAGLPA |
| | | | | | | RFYTSVELISCETYNSWLALQQKRLFKMLGIKRWLQAAE |
| | | | | | | SEFELLATTDFNPNTVLAKARDIQNEAMHRLNGQGSKQE |
| | | | | | | SLIGILLDMHDDTAENPLSRRAINHLLINHLKISEEEENLD |
| | | | | | | KLSERLDKKRVEIRRLEEQLNSRLPKGRDPTGQRYLQILS |
| | | | | | | YITSLPELSNDPQKLEAELDKLTIQQQLPLFNELPYPIRFES |
| | | | | | | SDDLYWSVLSQDSPNSSHTEHGVHQELPKSEKLQTKHRP |
| | | | | | | QPKERICVQFKGTQNFTFKIQCAHRQLPIFRQFLVDYQTY |
| | | | | | | KKLPETERFSQGLFALRSACLIWRKDDRKHGSKKKRAND |
| | | | | | | KQEDQLKPWNTHRLYLHCTVDRRLLTAEGTEQVCEEKK |
| | | | | | | NRAIQALKDKQKLEVSELQKLGLTKAQISDVERKRSTLN |
| | | | | | | RLENNPYPPRPNTIPYEGQPHIVIGVSFSRNEPVTIVIVDVE |
| | | | | | | KQEVLERQSAEELLNRGEPQYIWRNGKKEPLLRDGTERQ |
| | | | | | | HPNGGKLYIRKGKYAQCKPYRLVEQLHRQHQRNCKQRI |
| | | | | | | QEQKQDRYRETNSDSSLGLYVDRLIASRIVSLALQRKAG |
| | | | | | | TIAIPQLKGVRENVESDIRAKAERLFPNEKELQKKYAKDY |
| | | | | | | RDSFHRWSYARISECIRECAKKEGIAVVLRKQPSQGNLEE |
| | | | | | | KAKAIALSS |
| MG64 | 679 | MG64-117 | protein | unknown | uncultivated | YTYKSWLSSHKRDYNKLQGKKKWLAIMDHDLEVAKST |
| effectors | | effector | | | organism | DFSSQLIQTKAAEMLSQAYAQRENLQQNQTQQIKKKPSE |
| | | | | | | KLENCPSIMTILFGMYDKSQNSLECRALAHLLKNGCQVN |
| | | | | | | EEEEDTEALKFRLEKKRIQIARLEKQLESRLPDGRDPTGE |
| | | | | | | RFASNLDNAIALPEYSTFISSKFWANWYQALQTNPDNNS |
| | | | | | | QLLILFLSFISYKQNIAAEFDAWAQEEPQRLALTSTIYETL |
| | | | | | | PYPIRFGSTEDLYWSSEQVDPNPKATREERHARQCPKRR |
| | | | | | | RKPKHKQKVNERICVRFKGKGLENHRFRINCDRRQLPVF |
| | | | | | | KQFLTDRETQKQRKKAEKFMGGAFTLRSACLVWKKDTE |
| | | | | | | GLHRKRTSATSLLWMKFLFAIQWKETITDKELDSWSISLL |
| | | | | | | CINTTFPWQTHHLYLHCTIDSRLLTAEGTEEVIAGKAAAA |
| | | | | | | QQYLDESKLKASTSKRQAVERMDEEKQQTPQAKGETTA |
| | | | | | | KKTKTTLARSKNPPPARPSRPVYKGQSYVEAVVVVSRFA |
| | | | | | | IVAIAVVNTQTQEILEFQEVKDLLTEHRSDVLEKRLKKHQ |
| | | | | | | GMLKGRQSLEQLQLQQYRLVKRWRKHRKKNLTQRQEE |
| | | | | | | QKRGLYKRSNQESNLAQYLNRLLAKRIVQLCQTYQVGTI |
| | | | | | | VLPELGNIRENFECEIQAKARQKFPDDNVQLQKQYAKEV |
| | | | | | | RMRVHRWNYKNLLHAIRQCATHTRIPVTTRWQPKEGTL |
| | | | | | | RDKATIMTRPVPIGVP |
| MG64 | 680 | MG64-118 | protein | unknown | uncultivated | MLAENEFIIWLGMPMFTIQSRISASRETRQYFWEVMHKH |
| effectors | | effector | | | organism | TLLVNEVQEKIAQHSQFQEWQKKGNISRETVRGILAPLK |
| | | | | | | ENPSYTGLPGRFYSSAELISCYTYKSWLALQQQRQLRLL |
| | | | | | | GKKRWLQAVESEFELLATTNFNPDEVRVKAREIHNEAIQ |
| | | | | | | KLNGGGRKYKAPSLIGIVLEMHDRTAEAPLSRRAINHLLI |
| | | | | | | NNLKISEEEQNLEKLSEHLGKKRKEIERLEEQLISRLPKGR |
| | | | | | | DPTEQRYLETLYQVTALPELSNDPEKLATELKTLTVQQQ |
| | | | | | | LPLFKELPYPIQFGSSGDLYWSVETQGTSNPADAENGINQ |
| | | | | | | ELPKSKKRQKKCCKRPQERICVQFKGVIDHTFKIQCDRR |
| | | | | | | QLPIFRQFLIDYQTHQELPDEERFSEAEFALRSACLIWRKD |
| | | | | | | DTGQSSNKKRTSDEQEDQLKPWNTHRLYLHCTVERRLL |
| | | | | | | TAEGTEQVREEKKKEVIKTLKGQEKLQELELEQLGLTKT |
| | | | | | | QIEYVRRKRSTLNYLENNSPPPRPNAKPYEGQPHIVIGVSF |
| | | | | | | SRHEPVAIAVVDVKKEEVLECQNAKELLNRGKAQYIWR |
| | | | | | | NGKKEPLIKDGIEQRHPNGGKLNIRKGKRVRRKPHRLVQ |
| | | | | | | QLHQRHQQNSRRRSEEQKQDRYRSSNSDSNLGLYVERLI |
| | | | | | | ASKIVELALQRKASTIAIPQLKGIRESVESDIQARAERLFP |
| | | | | | | NEKERQKEYAKHYRASFHRWSYNRLSECIKECASSEGIA |
| | | | | | | VVIRQQSSVGELEQKAIAIALSFSNVKTS |
| MG64 | 681 | MG64-119 | protein | unknown | uncultivated | MSRNSKKNSTSPILQTIRCHLHASEDVLCKVWEEMTQKN |
| effectors | | effector | | | organism | TPLIVQLLSGVSEQPEFEVNKENGKISKPEITELRRFLTKD |
| | | | | | | SDLEKQSGRLRSSADTFVTEVYSSWLTLYQKRKSQKEGK |
| | | | | | | EYFVKNILKSDVELIAESNCDLQTIRDKAQEILAQPEKILK |
| | | | | | | QIVASDENSKQTNSNQKEDKKKSKKNSSTKQKSNIVAQQ |
| | | | | | | KDNDSKTLTNILYEIHKKTQDVLTRCAVAYLIKNHNKTS |
| | | | | | | DLKEDIEKLKERRTKKEVEIKRLEKQLQDNRLPNGRDITG |
| | | | | | | TTYAEAFDNLIKQVPKNHEECTTWIADLLKKISPIPYPINY |
| | | | | | | LYSDLSWYKNDKEQICIYFSGWAKYHFQICCNKRQRHLF |
| | | | | | | ERFLEDHIAFEASEKGEEKLSGSLVTLRSVQLLWQQAEG |
| | | | | | | KGEPWKVHKLALHCTYDARLWTAEGTEEVIKEKTDKAQ |
| | | | | | | KKVSNAEKNENLDNNQQTQLNKNKSSLSRLSNSFNRPSK |
| | | | | | | PVYQYQNNIIVGISFHPVELATVAVIDINTHKVLDNKTVK |
| | | | | | | QLLGDDYHLLSRRRRQQVHFRKEREKAQKKDASCNIGE |
| | | | | | | SELGEYVDKLLAKRIVEIAKHYRAASIVLPKLKDMREIRT |
| | | | | | | SEIQAKAETKFSGDINAQKLYIKEYNHQIHNWSYNRLQE |
| | | | | | | CIKSKAAQLRICIEFGIQPNYGTLQEKARDLAFSAYQSRT |
| | | | | | | NDIGK |
| MG64 | 682 | MG64-120 | protein | unknown | uncultivated | MSQKTIQAQLVATESSRQVIWQLMAERNTPLINALLEQIA |
| effectors | | effector | | | organism | TSSGFEEACSKGSISQSIVKECCQKLREDSQFSGQPGRFYT |
| | | | | | | SAIVTVSRIFKSWIHIRRKIIYQLEGQTRWLLILQSDEELTE |
| | | | | | | ACNCDLDILRSKAAEVLAKVESQTHQKGQFNILFELYRD |
| | | | | | | AKDSLTSGAIAYLLKNGSKLPDKPEDPKKFVKRRRKAEI |
| | | | | | | RAERLTTTLKRLRIPSGRDLTGQKWEETLAVAASSVPEN |
| | | | | | | EDEAALWQAVLLSETKKLPFPILYETNEDLTWFLNDEGR |
| | | | | | | LCVNFNGLSEHSFEIYCDQRQLHWFKRFFEDQETKKASK |
| | | | | | | NQHSSALFTLRSARLAWQKGEGKEELWNVHRLVLSCTV |
| | | | | | | ETRLWTAEGTEQVREEKATEYAKVIAGTKAKGNLNKNQ |
| | | | | | | EKFVQSREKTLALIKNPFPRPNRPLYKGQPTILAGVSYGL |
| | | | | | | DRPATLAIVDITTGKAITYRSIRQLLGDNYDLLNGYRLRQ |
| | | | | | | QRNAHRRHNRQRTGASNQIQESDQGEYLDCLIAQAIVST |
| | | | | | | AQDYQASSIVLPDLGNIREIVQAEVQARAEQKIVGYLEG |
| | | | | | | QKHYAKQYRASIHRWSYSRLNEKIQGQSAQIGIAIEQTKQ |
| | | | | | | ALQGTPQEKAKNLVLEAYNSRQESSLTRK |
| MG64 | 683 | MG64-121 | protein | unknown | uncultivated | MSIITIRCRLVAGVKQQIDKKSIKNFSAEDRALLQKLLDD |
| effectors | | effector | | | organism | KSHADRDNPKDKEKIFLAQSSEAVRQNLWQLFLTSSALI |
| | | | | | | DELLDRLSQHPNIHTWQQQGNLPDDELKACWLELKASP |
| | | | | | | LYDEKLPGRFFSSVQSMVKNIYASWLALNQQKQRRLNG |
| | | | | | | LNRLTEIAYSDEALLEMCDLTFTQLQANAESMLAEIDKEI |
| | | | | | | AGSEKPLSRINLLFQKYTELPDSDILGRSAIAYLIRHGCKIE |
| | | | | | | SKIEPAAEFKKWFKTKLKQARRLENQLAGHFPRGRDLNG |
| | | | | | | TAFLNVLEIATNDEPQDNQELMLWQSQILRDPSSLPHPIE |
| | | | | | | FNSNTDLRWLKLDRKQYKCQRVASGESIESIELTQRLFVE |
| | | | | | | FNGLTRGTNYVFEVYCDRRQLAIFQQFFNDDRLSRNSSS |
| | | | | | | DEKYSSSLFTLRSAHLLWDRNESQDRHRTLATQTADEPW |
| | | | | | | NSNQLYLHCAIETKSLTAEGMREIKQQKTQKVNNTLVKQ |
| | | | | | | SKNIDPSIDQQQSYRKNQTSLALLDRPLPRPSRPKYQGNP |
| | | | | | | QIIVGLIFDPVRPIYLAVVDVTTGKTITCRSTRQLLGDKHP |
| | | | | | | KLSEYRLKQQQNSSLRRKQNQQGQFNQPTESTQGEHLD |
| | | | | | | RLLAKAVIRVAQEFKAASIALPPVNNSIEKNQSELEAYAE |
| | | | | | | EEIPEDIVTQQQLTRKTSVVIHKWSYNRLSGYIRNNATKL |
| | | | | | | DIAVETASSPSPGTPLQQAAESAISAYNSRKHIKK |
| MG64 | 684 | MG64-122 | protein | unknown | uncultivated | MKDVGTLLIEIKKPVGDVPLVYKRLRALTRSGAQALNM |
| effectors | | effector | | | organism | TMQDSHPNAVAQLRAVWDGEAKQTRTSSEDWRARVRT |
| | | | | | | VLARNWNARLERERLDRKAEPDAEMVYAGINGSALAEE |
| | | | | | | TATNITSQFNGENMKEMIALRKGFPEFGIASSFYCGGRYC |
| | | | | | | AVVGTLRETLKRTCATKDDADAAVKELGEGHFARRAGD |
| | | | | | | KWQVVHEDSARVALPLWGTGKKVTELIIGPAGGHVRST |
| | | | | | | WRQLVAHFERRDEIVQAEKELDAIYRTEAEKAALASLKA |
| | | | | | | VKKTERDKTKRREIDGQIRAVVEASNKRMQSLRDPIEAK |
| | | | | | | LYGLTKIGRIGVVWKERRRKWFVTISYTRYTPSIETKGQA |
| | | | | | | AAVNFGINVFLQAFAADESAFHVDGAQILHKRLAYNAA |
| | | | | | | RKRIQKSKRQFGRGSRGRGKRRRELPLTKRQGDETNWT |
| | | | | | | QTFIRQLASDLIAWCKRRGVADLYLEDLSGVRDEFEQAT |
| | | | | | | GGQAHPEVKRRIHSWPYAETRMAIVREGTQHGVRVHAK |
| | | | | | | GSRFVSQRCPSCTHTVPENVQEVTIPGPVLPLQSLGKGRW |
| | | | | | | REVPYQRFQDSESKAAQYDLYRRQDKTYRFECVACGMK |
| | | | | | | GQADIVACLNHLQDLGVVPAGKPTPPGTPTPLAKMQEA |
| | | | | | | ARASVSNDKRKKVRRTGT |
| MG64 | 685 | MG64-123 | protein | unknown | uncultivated | MKDVGTLLIEIKKPVGDVPLVYKRLRALTRTGAQALNM |
| effectors | | effector | | | organism | TMQDSHPNAVAQLRANWDGEAKQTRTLSEAWRGQVRT |
| | | | | | | VLARNWNARLERERLDRKVEPDAEMVYAGINGSALAEE |
| | | | | | | TATNITSQFNGANVGEMIALRKGFPEFGIATSFYCGGRYC |
| | | | | | | SVAGTLRETLKRTCATKDDADAAVKELGEGHFARRAGD |
| | | | | | | KWRVVHEDSARVALPLWGTGKKVTELIIGPAGGHVRST |
| | | | | | | WRQLAAHFDRRDDIVLAEKQLDAIHRTEAEKEALASLK |
| | | | | | | AARKAERDRKKRREIDGQIQAVVGAMNKRMQSLRDPIE |
| | | | | | | AKLYGLTKIGRIGVVWKERRRKWFVTISYTRYTPPIETKG |
| | | | | | | QAAAVNFGINVFLQAFAADESAFHVDGAQILHKRLAYN |
| | | | | | | AARKRIQKSKRQFGRGSRGRGKRRRELPLTKRQGDETN |
| | | | | | | WTQTFIRQLASDLIAWCKRRGVADLYLEDLSGVRDEFEQ |
| | | | | | | ATGGQAHPEVKRRIHSWPYAETRMAIVREGTQHGVRVH |
| | | | | | | AKGSRFVSQRCPSCTHTVPENVQEVTIPGPVLPLQSLGKG |
| | | | | | | RWREVPYQRFQDSESKAAQYDLYRRQDKTYRFECVACG |
| | | | | | | MKGQADIVACLNHLQDLGVVPAGKPTPPGTPTPLAKMQ |
| | | | | | | EAARASVSNDKRKKVRRTGT |
| MG64 | 686 | MG64-124 | protein | unknown | uncultivated | MSVITIQCRLVASEETRRYLWSVMVEKNTPLINELLKRV |
| effectors | | effector | | | organism | KQHPDFEKWRRRGKPSKEAVRELCEPLRNDPQFNGQPG |
| | | | | | | RFYTSAIATVQQIYESWMATQLGLQRSLDGKVHWLEVV |
| | | | | | | ESDAELVTQSNVDADAIRAKAREILLDIASGCLPQKTQEK |
| | | | | | | KAKASQNKEKPRQGKKQQKEKPEVPQNLVGVLLDFYDE |
| | | | | | | TKEVVKRRALIHLLRNDCEVNEKEDAKKLIDKLNNKRAE |
| | | | | | | IERLKERLEGRLPKGRDLTDQDFLEALEIATSIPADDTLSE |
| | | | | | | FLAWENKVIPNLPKLVKAPKSMPYPIYYETNTDFNVWKR |
| | | | | | | NEKGRICFELNGLSNHIFEVYCDRRQLHFFEQFLRDYETK |
| | | | | | | KTGGKQHSTGLFLLRSAQLLWCEDKTRIKKKKVKVQNP |
| | | | | | | LGGKAKKKAEKTKIVEPWNYNYLSLHCAVDTRLLTEEG |
| | | | | | | TQQVREELKEDIAETLERQRTKLSNLSDEQVEEKKNLRQ |
| | | | | | | SIQSKESTLSRSSGSFSRPSRIPYSQFSRSNVLVGVCFEFHN |
| | | | | | | LVTVAVVDASQNQVLAYRSTRQLLTNERVEIPEAAEGSA |
| | | | | | | KRVSRRLRYQNYRLLNRHRQRQQRNARQRREDRKRRIY |
| | | | | | | RNSPKSQSGQHLDRLIASSIISLAQKHRAGSIVLPETKGLL |
| | | | | | | ERTEGKVRALAEQKVPDYKQGQEKFAKQHRKRYNRWN |
| | | | | | | YARLLQTVQQQAAKLGISVELGRQISEGSSQEKARDLAL |
| | | | | | | AIYHSRQVVDA |
| MG64 | 687 | MG64-125 | protein | unknown | uncultivated | MSQITIQCRLVSSPGTRQQLWTLMAERNTPLINALIEQISQ |
| effectors | | effector | | | organism | HPEFEIWRRKGKISSTLVAQLCKSLKTEVRFNGQPARFYT |
| | | | | | | SAEHAADYIFKSWLAIQKQLQQRLDGKLRWLEMLKSDE |
| | | | | | | ELAQAGEVELNVIRDRASAILSQLQPTTPNDESPSHTKKK |
| | | | | | | GKQIKKKVASTDRSLVSQLFDRYRDSKQVLERCAIAFLL |
| | | | | | | KNGCKIPQDNEDPQKFAQQRRKAEIQVKRLQEQIESRIPH |
| | | | | | | GRDLTGASWLNTLETATQTVPKDNAEAKRWQNRLLTNP |
| | | | | | | SVLPFPLIFETNEDLVWKRNAKGRLCVHENGLSDYTFAIY |
| | | | | | | CDNRQLHWFQRFLEDQETKRSSKNQHSSGLFTLRSARLA |
| | | | | | | WQVGEGKGNPWDVHQLTLYCTIDPRLWTAEGTEQVRQ |
| | | | | | | EKAAEVAKKITQMESKGDLLVTQQVYVKRLNSTLTRINT |
| | | | | | | PFDRPSKPLYQGQSHIVVGLSLGLEKPATVAVWNADTNQ |
| | | | | | | VLAQYGIRQLLGENYRLFTRRRTEQLKTAHQRHKAQKR |
| | | | | | | EAPKQLGESELGQYVDRLLAKSIVTIAKTFKSGSIAVPKL |
| | | | | | | GNIREIVEAEIKAKAEEKCPGFVEGQRKYAKQYRTNVHR |
| | | | | | | WSYGRLIESIRSQATKLGIVIEEAKQPLTGKSEEKAKDVAI |
| | | | | | | AAYQART |
| MG64 | 688 | MG64-126 | protein | unknown | uncultivated | MKDESTSVGASSLLQHGATPRPEGEEVSGHVPLRRKEAS |
| effectors | | effector | | | organism | PETLAKSGTSVPTRSDPAATPEGGEEAPTDVDSGGGSGG |
| | | | | | | HGRSAKRRPKGKRTSIDGKPPDGIVEGEPLPEPTITRATTK |
| | | | | | | FSIRLDALSENLNRARKKGQKRARSLAEDEAREALIVASR |
| | | | | | | YATTAQNVGMRLLYRADSDVLDAFMAEHGRLPIPGEVI |
| | | | | | | WPSAKAYRADSPVYVYRRMVMAVPELSSSIAATLSKKV |
| | | | | | | VEKWASERVDTLVRQQRSAPHYRLGGPLPIPAQVTQWT |
| | | | | | | WDPSSKRAMVDVTLFSTKKRKGVHRVTIPVEARDDRQA |
| | | | | | | KELEMLALGDGNPAVGWRPGEVTIQRDRLRPQKWFLRC |
| | | | | | | AYTRVAVARKEGVAAAINLGMRCLLAFYVEGGPSDIYE |
| | | | | | | ARDIEAYLRGIQRRRRDRQNVYRWTDGSRRGHGRPRAL |
| | | | | | | QSIEVLAERAANYRKTRMQTLARRFARRLVEMGVNVLY |
| | | | | | | VQKFDGIRDALPELLMASMPRSQWIWERIQEWPYSEMQ |
| | | | | | | ARIVTCCQDEGIRCVEMAAQYNSQRCPVCGFVSKDLRDL |
| | | | | | | VNWRLKHAGCINRHLDVGHAMNAIARGAATDPDGSRKI |
| | | | | | | KGLAEWNLNLDETARETSGKKAKEKGNGETGGDG |
| MG64 | 689 | MG64-127 | protein | unknown | uncultivated | MSIKTIECRIVAEPESLKALWIIMTQKNTPLINELLAQVPN |
| effectors | | effector | | | organism | HPDFEQWLEYGNLPQKPIKELCAPLREKEPFANQPGRFY |
| | | | | | | TSAIALVHYIFKSWLNVQKKLRQRIEGKQRWLDMLKSD |
| | | | | | | RELQDESGKDLAEIRLLAVEILQQIEQHLACQKVNLKKK |
| | | | | | | QKKRKKTKSKKTSSTIFNSLHERYDKTDDYATKCAIVYL |
| | | | | | | LKNGCQISEAPEDEEDPEEYALGRRKKEIEIENLQKQLRS |
| | | | | | | RRLPSGRDLKHEVYSQALEAIEDNHVPEDNTESKLLQDN |
| | | | | | | LSRKSVAVPYPVAYESNTDLTWLEDKNGNLSVKFNGLG |
| | | | | | | KHEFEIRCDTRQIDWFRRFLEDQKLKEKSKQDKKKGLRD |
| | | | | | | SEISSALFGLRSGRILWRKGEQKSDRRKKTLDRAFFLLSL |
| | | | | | | SKDYKLALALLQGYGQYKRRLRQEQPWTLHRLYLQCSI |
| | | | | | | ETKLWTQEGTKLVAKQKNTKSNNTITQTKEKGDLDSEQ |
| | | | | | | QKHIKRKQAQLANLKNIPQRPCKDLYKGNSSMILGVSLG |
| | | | | | | LEKPVTVAVVDVVSGRVLTYRSAKQLLGKNYKLLNRQR |
| | | | | | | QEKLRLSIKRHKSQKRNASNNFGESNLGKYVDRLFADAII |
| | | | | | | KLAQSFKVSSIAIPEVRQIREITTSEVRAKAERKIKGYKEG |
| | | | | | | QKKYAKQYRVNVHNWSYGRLTNFISQKAGIYGITIEKAR |
| | | | | | | QPLGSSNQEQARNIALSVYNSRLENVV |
| MG64 | 690 | MG64-98 | nucleotide | artificial | | GGTCGCAATGGCCGTTTTGGCCGGAGAAGGGATGAAA |
| effectors | | crRNA | | sequence | | G |
| crRNA | | sequence | | | | |
| sequence | | | | | | |
| MG64 | 691 | MG64-99 | nucleotide | artificial | | GTCGCAAAACCCTACCCTGGCCAGGGTGGGTTGAAAG |
| effectors | | crRNA | | sequence | | |
| crRNA | | sequence | | | | |
| sequence | | | | | | |
| MG64 | 692 | MG64-100 | nucleotide | artificial | | GTTTCAACAACTATCCCGGCTAGGGGTGGGTTGAAAG |
| effectors | | crRNA | | sequence | | |
| crRNA | | sequence | | | | |
| sequence | | | | | | |
| MG64 | 693 | MG64-101 | nucleotide | artificial | | GTTTCAACACCCCTCCCAGCGAGAGGCGGGTTGAAAG |
| effectors | | crRNA | | sequence | | |
| crRNA | | sequence | | | | |
| sequence | | | | | | |
| MG64 | 694 | MG64-122 | nucleotide | artificial | | GTCGGGATGGATCTGGAGAAGGAATGGTGTTGGAAG |
| effectors | | crRNA | | sequence | | |
| crRNA | | sequence | | | | |
| sequence | | | | | | |
| MG64 | 695 | MG64-126 | nucleotide | artificial | | GTGGCGACGGGTGAGGAGGCCGGATCGGGTTGGAGG |
| effectors | | crRNA | | sequence | | |
| crRNA | | sequence | | | | |
| sequence | | | | | | |
| MG64 | 696 | MG64-102 | nucleotide | artificial | | GTCGCAATCGCTCTTCCAGAAATGGGGGGGCTGAAAG |
| effectors | | crRNA | | sequence | | |
| crRNA | | sequence | | | | |
| sequence | | | | | | |
| MG64 | 697 | MG64-124 | nucleotide | artificial | | GTCGCAATCGCTCTCTCGAACAGGGGGAGATTGAAAG |
| effectors | | crRNA | | sequence | | |
| crRNA | | sequence | | | | |
| sequence | | | | | | |
| MG64 | 698 | MG64-114 | nucleotide | artificial | | GTCGCAATGGCCCTCCTAGTGATAGGTGGGCTGAAAG |
| effectors | | crRNA | | sequence | | |
| crRNA | | sequence | | | | |
| sequence | | | | | | |
| MG64 | 699 | MG64-108 | nucleotide | artificial | | GTTTCATCAACCCTCCCGCCTTGGGGTGGGTTGAAAG |
| effectors | | crRNA | | sequence | | |
| crRNA | | sequence | | | | |
| sequence | | | | | | |
| MG64 | 700 | MG64-110 | nucleotide | artificial | | GTTTCATCGACCCTCCCGCATTTGGGTGGGTTGAAAG |
| effectors | | crRNA | | sequence | | |
| crRNA | | sequence | | | | |
| sequence | | | | | | |
| MG64 | 701 | MG64-115 | nucleotide | artificial | | GTTTCATCAACCCTCCCGCCTATGGGTGGGTTGAAAG |
| effectors | | crRNA | | sequence | | |
| crRNA | | sequence | | | | |
| sequence | | | | | | |
| MG64 | 702 | MG64-106 | nucleotide | artificial | | GTTTCATCAACCCTGCCGCAACAGGGTGGGTTGAAAG |
| effectors | | crRNA | | sequence | | |
| crRNA | | sequence | | | | |
| sequence | | | | | | |
| MG64 | 703 | MG64-117 | nucleotide | artificial | | GTTGCAATCGCCCTCCCGCCAACGGGTGGGTTGAAAG |
| effectors | | crRNA | | sequence | | |
| crRNA | | sequence | | | | |
| sequence | | | | | | |
| MG64 | 704 | MG64-104 | nucleotide | artificial | | GGGCGCAACAGCCCTTTTAGCCACGGGTGAGTTGAAA |
| effectors | | crRNA | | sequence | | G |
| crRNA | | sequence | | | | |
| sequence | | | | | | |
| MG64 | 705 | MG64-111 | nucleotide | artificial | | GGCGCAACAGCCCTTTTAGCCACGGGTGAGTTGAAAG |
| effectors | | crRNA | | sequence | | |
| crRNA | | sequence | | | | |
| sequence | | | | | | |
| MG64 | 706 | MG64-109 | nucleotide | artificial | | GTTTCAATGACCATCCCAACTAGGGGTGGGTTGAAA |
| effectors | | crRNA | | sequence | | |
| crRNA | | sequence | | | | |
| sequence | | | | | | |
| MG64 | 707 | MG64-120 | nucleotide | artificial | | GTCGCGATCGCCGTTTCAGCCTTGGGCAGATTGAAAG |
| effectors | | crRNA | | sequence | | |
| crRNA | | sequence | | | | |
| sequence | | | | | | |
| MG64 | 708 | MG64-125 | nucleotide | artificial | | GTCGCAATCACCTGTCCGAATTAGGGCAGGTTGAAAG |
| effectors | | crRNA | | sequence | | |
| crRNA | | sequence | | | | |
| sequence | | | | | | |
| MG64 | 709 | MG64-107 | nucleotide | artificial | | AGTCGCAATCGGTCTCCCAGAGATGGGCGGGCTGAAA |
| effectors | | crRNA | | sequence | | G |
| crRNA | | sequence | | | | |
| sequence | | | | | | |
| MG64 | 710 | MG64-103 | nucleotide | artificial | | GTTGCAATAGCCCTCCTAAATTAGGGTGGGTTGAAAG |
| effectors | | crRNA | | sequence | | G |
| crRNA | | sequence | | | | |
| sequence | | | | | | |
| MG64 | 711 | MG64-118 | nucleotide | artificial | | GTTTCATTAGCCTTCCCGCTTTTGGGTAGGTTGAAAGA |
| effectors | | crRNA | | sequence | | |
| crRNA | | sequence | | | | |
| sequence | | | | | | |
| MG64 | 712 | MG64-127 | nucleotide | artificial | | GTTGATTAGACCTTCCCGAACTGGGATGGGTTGAAAG |
| effectors | | crRNA | | sequence | | A |
| crRNA | | sequence | | | | |
| sequence | | | | | | |
| MG64 | 713 | MG64-113 | nucleotide | artificial | | AGTTTCAATACCCCTTCCGGCTAGGAGTAGGTTGAAAG |
| effectors | | crRNA | | sequence | | |
| crRNA | | sequence | | | | |
| sequence | | | | | | |
| MG64 | 714 | MG64-121 | nucleotide | artificial | | AGTTGCAATACTCCTTCTGACAAAAGGTGGTTTGAAAG |
| effectors | | crRNA | | sequence | | |
| crRNA | | sequence | | | | |
| sequence | | | | | | |
| MG64 | 715 | MG64-98 | nucleotide | artificial | | CCAGGTGAATGAGTAAGATCCCCCAAGGATCTAAACC |
| effectors | | predicted | | sequence | | TTGAGCAGTCTTCTGCTGAGGTTGGGCATGGTTCTCAT |
| putative | | tracrRNA | | | | TGTGGCTACTGACCTTCTCGATTTTTTGGCAAAGCGGA |
| tracrRNA | | | | | | GCGGGGCAAAAATCCCGGTATCCTTGCCAAGTTCCTCA |
| | | | | | | TTTAGTGCCAGCCATGGGTTTGCGCTCGCGCTCATTGA |
| | | | | | | ATCGTTGCTAGTCAGCGAAGCGCAAAAGCGATGCAAA |
| | | | | | | CATCAGGCTTTGCCAATGCGAGGCTTGGAAAGGTTGC |
| | | | | | | AGCACATTGAATGGA |
| MG64 | 716 | MG64-99 | nucleotide | artificial | | TTTCTAATAGCGCCGCGACTCATGCTCTGCCTCTGAGT |
| effectors | | predicted | | sequence | | CGTGTTAAATGAGGGTTAGTTTGACTGCCGGAAGGCA |
| putative | | tracrRNA | | | | GTTTTGCTTTCTGACCCTGGTAGCTGCTCACCCTGATG |
| tracrRNA | | | | | | CTGTCGGATAAGAAGCTTAGGCTTTTTAGAAGAGATTA |
| | | | | | | AGTCTGATAGAACATTGCTTTCCTATTATGACGTAGGT |
| | | | | | | GCGCTCCCAGCAACAAGGGCGCGGATATACTGCTGTA |
| | | | | | | GTGGCTACTGAATCACCTCCGATCAAGGGGGAACCCG |
| | | | | | | CC |
| MG64 | 717 | MG64-100 | nucleotide | artificial | | AGAATAATAGCGCCGCAGTTTATGTTCTTTAGAGCCAA |
| effectors | | predicted | | sequence | | TATACTGTGAAAAATCTGGGTTAGTTTGACGGTTGTCA |
| putative | | tracrRNA | | | | GACCGTTTTGCTTTCTGACCCTAGTAGCTGCCCGCTTCT |
| tracrRNA | | | | | | GATGCTGCTGTCGCAAGACAGGATAGGTGCGCTCCCT |
| | | | | | | GCAATAAGAAGTAAGGCTTGAAAAAGTAATAGCCGTT |
| | | | | | | GCTAGCAACGGTGCGGGTTACCGCAGTTGGTGGCTAC |
| | | | | | | TGAATCACCCCCTTCGTCGGGGGAACCCTCC |
| MG64 | 718 | MG64-101 | nucleotide | artificial | | AAAATTCAACAGCGCCGCAGTTCATGCTTGTTATAAGC |
| effectors | | predicted | | sequence | | CTCTGTGCTGTGTAAATTTGGGTTAGTTTGACTGTTGTT |
| putative | | tracrRNA | | | | AAACAGTCTTGCTTTCTGACCCTGGTAGCTGCCCACCT |
| tracrRNA | | | | | | TGATGCTGCTGTCCCTTGTGGATAGGAATTAGGTGCGC |
| | | | | | | CCCCAGTAATAGAGGTGCGGGTTTACCGCAGTGGTGG |
| | | | | | | CTACTGAATCACCTCCGAGCAAGGAGGAACCCACCTT |
| | | | | | | AATTATTTTTTGGCG |
| MG64 | 719 | MG64-102 | nucleotide | artificial | | GATTCAACAGCGCCGCAGTTCAAGCTCTTTGAGCCGCT |
| effectors | | predicted | | sequence | | GAACTGTAAAATGGGTCAGTTTGGTCGTTGTGAAACG |
| putative | | tracrRNA | | | | GCTGTACTTTCCGACCCTAGTGGCTCTCCGCTCACTGA |
| tracrRNA | | | | | | CTGCCATCCTGGTACAGGTTTCATGAGGAATTTGTGTG |
| | | | | | | GGGATGGGAAGCTGCATTGGTTGAGTTCGTTCTTCAAA |
| | | | | | | TGTAGCGCAGGTGCGCGCCCAGCAGAAGTGAGTCAAG |
| | | | | | | CCTTCATTCTGTGAAGGTACGGGAGCGTTGTCTCTTCA |
| | | | | | | AGTAGTTGATGCTGATGACGTGACCGGAGCGACAGCC |
| | | | | | | ACTGAATCGCCTCCGATCAAGGAGGAGTCCTCC |
| MG64 | 720 | MG64-103 | nucleotide | artificial | | TGTTTAATAGCGCCGCAGGTCATGCTTTTCGGAGCCTC |
| effectors | | predicted | | sequence | | TGAACTGTGATAAATGAGGGTTAGTTTGACTGTTGTGA |
| putative | | tracrRNA | | | | GACAGTCTTGCTTTCTGACCCTGGTAGCTGCTCACCCC |
| tracrRNA | | | | | | GATGCTGCTGTCACATGACAGGATAGGTGCGCTCCCA |
| | | | | | | GCAAAAAGGGTGCGGGTATACTGCTGTAGTGGCTACT |
| | | | | | | GAATCACCCCCGACCAAGGGGGAACCCTCC |
| MG64 | 721 | MG64-104 | nucleotide | artificial | | ACAATAATGCTGGCGACCCAGTTAGTGCTTTAGGCAC |
| effectors | | predicted | | sequence | | GAACAAGTGGAAAAGGTCAGTTTTACCCTTGGGTGCTT |
| putative | | tracrRNA | | | | TCCGACCTGTGTACTGTCCGCTATTCATGCTGCTGCCT |
| tracrRNA | | | | | | AATAAGGCAATTCGCCACAGCAATGAATAGCACCGCT |
| | | | | | | CTACCGCCTTAAAAAAGCGGTACAGTAATGCAGATGT |
| | | | | | | GGCAGTCCAAATCGCCTACTGAATACGGTAGGATCTC |
| | | | | | | CCCACAAATTTTT |
| MG64 | 722 | MG64-108 | nucleotide | artificial | | AAATTAACAGCGCCGTCGTTCATGCTCTTCGGAGCCTC |
| effectors | | predicted | | sequence | | TGTACGGTGAAAAATCTGGGTTAGTTTGACTATCGGAA |
| putative | | tracrRNA | | | | GATAGTTTTGCTTTCTGACCCTAGTAGCTGCCCGCTCC |
| tracrRNA | | | | | | TGATGCTGCTGTCTATCGAATTGTTCGTCCAGACGGGA |
| | | | | | | AATCTTAGCTCTAAATATCTAAAGAGTATTTTACTTCT |
| | | | | | | AGGATATCCAGAGATAAGAGAGGTGCGCTCCCAGCAA |
| | | | | | | TAAGGAGTAATGCATGACTTGCACTAGCCCTTGGTAAC |
| | | | | | | AAGGGTGCGGATAACCGCAGTGGTGGCTACTAAATCA |
| | | | | | | CCCCCTTCATCGGGGGAACCCTCC |
| MG64 | 723 | MG64-110 | nucleotide | artificial | | TATTCAATAGCGCGTCTTGTTCCCCGCGAACAGATGAT |
| effectors | | predicted | | sequence | | AAGTGTCAGGGCAGTTTAATTGCTTTCCGCCCTTGGTA |
| putative | | tracrRNA | | | | GTTGTCCGCGTCTCTCTAGAAATTGGAGAGACACGTCC |
| tracrRNA | | | | | | TTTACGGAGCTTGCTGTGTAATCGACGTTCCTTCTACG |
| | | | | | | GTACTTTTCCGTAGATCTGGTGCAGGGTCGCGCCTAGC |
| | | | | | | ATCAAGGAGCTATGTTTTTATAACCAGTGGTACAACGC |
| | | | | | | TTCTGGTTATAAGGATACAGGAGTACGTGTCGTGGCA |
| | | | | | | GCTACCCAATCGCCTTCGAGCAAGAAGGAATCCTCC |
| MG64 | 724 | MG64-112 | nucleotide | artificial | | TATTCAATAGCGCGTCTTGTTCCGTGAGAGCAGATAAT |
| effectors | | predicted | | sequence | | AAGTATTAGGGCAGTTTAATTGCTTTCCGCCCTTGGTA |
| putative | | tracrRNA | | | | GTTGTCCGCGTCTCTCTAATCTTTAGAGAAGAGAGACA |
| tracrRNA | | | | | | CGTCGGGAGCAGAGCTTACTCTGTAATCGACGTTCCTT |
| | | | | | | CTACGGAATTTTTTCGTAGATACGGTGCAGGCTCGCGC |
| | | | | | | CTAGCATCAAGGAGCTATGTTTTTATAATCAGTGGTAT |
| | | | | | | AACGCCTCTGGTTATAAGGATACAGGAGTACGTGTCG |
| | | | | | | TGGTAGCTACCCAATCGCCTTCGAGCAAGAAGGAGTC |
| | | | | | | CTCC |
| MG64 | 725 | MG64-113 | nucleotide | artificial | | ATGATACTAATGCGCCGTGGTTCATGCTCGAATAGAGC |
| effectors | | predicted | | sequence | | CAATGTGCTGCGTCTGAGTGTGGGTTAGGTTAATTGCT |
| putative | | tracrRNA | | | | TTCTGACCCAGGTAGCTGCCAGCCCTGAAGGTGGTATG |
| tracrRNA | | | | | | CGCTTTCACCAATAGGGGTGTTGATGTACTTCCGCAGC |
| | | | | | | GGCTACTGAATCACCCCTGAGCAAAGGGGAATCCACT |
| | | | | | | CCAATTTTTTGATTT |
| MG64 | 726 | MG64-106 | nucleotide | artificial | | GAATTAACAGCGCCGACCCTTCATGCTCTTCGGAGCCA |
| effectors | | predicted | | sequence | | ATGTAGGTGAAAAATGGGTTAGTTTGACTATCGGAAG |
| putative | | tracrRNA | | | | ATAGTCTTGCTTTCTGACCCTAGTAGCTACCCGCTTCT |
| tracrRNA | | | | | | GATGCTGCCGTCTATTGAATTGTCGTCCAGACGGGAAA |
| | | | | | | TCTTAGCTCTAAATATCTAAGATAGTATCTTACTTCTA |
| | | | | | | GGATATCCAGAGATAGGAGAGGTGCGCTCCCAGCAAT |
| | | | | | | AAGGAGTAATGCATGACTTGCACTAGCCCTTGGCAAC |
| | | | | | | AAGGGTGCGGATAGCCGCAGTGGTATCTACTGAATCA |
| | | | | | | CCCCCTTCATCGGGGGAACCCTCC |
| MG64 | 727 | MG64-114 | nucleotide | artificial | | ATTTGAATGATGATTAGCTTGTTGGGTCAGTTTGACTG |
| effectors | | predicted | | sequence | | TTGTGAGACAGTTATGCTTTCCGGCCCTGGTAGCTGCC |
| putative | | tracrRNA | | | | CGCTCGCTGACTGCCATCCTGGTACGTGTTCTCTTGCA |
| tracrRNA | | | | | | GATGCACTATGGGGATGGGAAGTTGCAGTTGTAGAAT |
| | | | | | | CTTTCTTTAACTGTAGCGTAGGTGCGCGCCCAGCAGAA |
| | | | | | | GTGAGTCCAGCCTTCCATAAAGAAGGTACAGCCTAGC |
| | | | | | | CCTGTAGTGGTGGCTACCGAATCGCCTCCGAGCAAGG |
| | | | | | | AGGGTCCTCCAAATATTTTTGGCAAAC |
| MG64 | 728 | MG64-115 | nucleotide | artificial | | TATTCAATAGCGCGTCTGGTTCCCTGAGAACAGATGAT |
| effectors | | predicted | | sequence | | AAGTGTAAGGGCAGTTTAATTGCTTTCCGCCCTTGGTA |
| putative | | tracrRNA | | | | GTTGTCCGCGTTTCTCTAGAGATTAGAGAGACACGTTA |
| tracrRNA | | | | | | CTTGCGGAGCTTGCTCTGTAAGCGACGTTCCTCTACGG |
| | | | | | | ATGTCAATCCGTAGATATGGTGCAGGGTCGCGCCTAG |
| | | | | | | CATCAAGGAGCTATGTTTTTATAACCAGCGGTTTACGA |
| | | | | | | TCTCTGGTTATAAGGATACAGGAGTACATGTCATGGCA |
| | | | | | | GCTACCCAATCGCCTTCGAGCAAGAAGGAGTCCTCC |
| MG64 | 729 | MG64-116 | nucleotide | artificial | | TATTCAATAGCGCGTCTTGTTCCGTGAGAGCAGATAAT |
| effectors | | predicted | | sequence | | AAGTATTAGGGCAGTTTAATTGCTTTCCGCCCTTGGTA |
| putative | | tracrRNA | | | | GTTGTCCGCGTCTCTCTAATCTTTAGAGAAGAGAGACA |
| tracrRNA | | | | | | CGTCGGGAGCAGAGCTTGCTCTGTAATCGACGTTCCTT |
| | | | | | | CTACGGAATTTTTTCGTAGATACGGTGCAGGCTCGCGC |
| | | | | | | CTAGCATCAAGGAGCTATGTTTTTATAATCAGTGGTAT |
| | | | | | | AACGCCTCTGGTTATAAGGATACAGGAGTACGTGTCG |
| | | | | | | TGGTAGCTACCCAATCGCCTTCGAGCAAGAAGGAGTC |
| | | | | | | CTCC |
| MG64 | 730 | MG64-117 | nucleotide | artificial | | TTTTGACGCGCTGCTATAGCAGCTAAATGGGTCAGTTT |
| effectors | | predicted | | sequence | | CAGTACTTTCCGTCCCAAGTAGTTGTCCGCTTCTGTCA |
| putative | | tracrRNA | | | | AGTAGTGCGATACAGCTTTCCTGTTTGATGCAGGAAAA |
| tracrRNA | | | | | | AGCACTTACAAGACGCGGGGTGAGCTTGCCTCAGTCG |
| | | | | | | GGGTGAGCATGCGTCAGCATCTCAGGAAGCAGTTCTT |
| | | | | | | AGGAATACAAGGATACTTACTTCTTAAGGATACAGGG |
| | | | | | | ATACGTGTGATATCTACAACTTCATTGTGTTGTTGCAA |
| | | | | | | CATAGAATTTGTAGGTGGACAGCTACTATACGCCCCA |
| | | | | | | AGCATGGGTTGA |
| MG64 | 731 | MG64-109 | nucleotide | artificial | | AAATAGTTAATAGCGCCGCTGTTCATGCTTCTCGGAGC |
| effectors | | predicted | | sequence | | CTCTGAACTGTGCAAAATGCGGGTTAGTTTGGCTGTTG |
| putative | | tracrRNA | | | | TCAGACAGTCTTGCTTTCTGACCCTGGTAGCTGCCCAC |
| tracrRNA | | | | | | CCCGAAGCTGCTGTTCCTTGTGAACAGGATATTAGGTG |
| | | | | | | CGCCCCCAGTAATAAGGGTGTGGGTTTACCACAGTGG |
| | | | | | | TGGCTACTGAATCACCTCCGAGCAAGGAGGAACCCAC |
| | | | | | | TTTAATTTTTTTCGTAAAG |
| MG64 | 732 | MG64-118 | nucleotide | artificial | | TATTTAATAGCGTGCCTCGTTCCCCGTGAACAGGTAAG |
| effectors | | predicted | | sequence | | AAGTGTAAGGGCAGTTTAATTGCTTTCCGCCCTGTGTA |
| putative | | tracrRNA | | | | GTTGTCCGCGTCTCTCTAATTTGTTAGAGAGACACGTC |
| tracrRNA | | | | | | GTTAACGGAGCTTGCTCCGTGAATGACGTTCCTTCTGC |
| | | | | | | GGAACTTTTCCGTAGATATGGTGCAGGGTCGCGCCTAG |
| | | | | | | CATCCAGGAGCTATGTTTTTATAACAGTCGTACAACAC |
| | | | | | | TTCTGGTTATAAGGATACAGCTTTACGTGTCGTGGCAG |
| | | | | | | CTACCGAATCGCCTCCGAGCAAGGAGGAGTCCTCCCC |
| | | | | | | ATCTATTTTTTGACG |
| MG64 | 733 | MG64-119 | nucleotide | artificial | | CATAATACATGCTCTTTTGAGCTTCTGTATAATGCTAC |
| effectors | | predicted | | sequence | | AGTATTAACCCTTTTGTAGATACTGTGGAATGGGTTAG |
| putative | | tracrRNA | | | | TTTAACGCTTGAAAAAGCGTATTCTTTCTGACCCTAGT |
| tracrRNA | | | | | | AGCTGCCAACTCTACCCGTGTGGTCATCTGATTGTTTG |
| | | | | | | TTAGCAGTAATTGCTTGGGTAAGCAGATGCTGTTTTTA |
| | | | | | | G |
| MG64 | 734 | MG64-120 | nucleotide | artificial | | ATAATTCTATTACGCCACGGCTCATGCCAGTAATGGTC |
| effectors | | predicted | | sequence | | TCTGTGCTGATGCTAAACGAGTTAGGTTGACTATTGGA |
| putative | | tracrRNA | | | | AGATAGTCTTGCTTTCTGGCTCTGGCGACTGTCCACCT |
| tracrRNA | | | | | | CAGAAGTTGGGTGCGTTCCCAGCAAAAAGGTGCGGGT |
| | | | | | | CTACCGCAGTGGTGGTTGCCGTCTTCACCTCCGAGCAA |
| | | | | | | GGAGGAATCTACCCTAAAAATTT |
| MG64 | 735 | MG64-121 | nucleotide | artificial | | AATGTGATTGCGCCTCGATAGATGCTCTATGAGCCGCT |
| effectors | | predicted | | sequence | | CGGTCGTAGAAAAATGGGTGAGTTTGACTATCTACTTC |
| putative | | tracrRNA | | | | GTTAGATAGTGCTGCTTTCCGACCCTGGCATTCTGTCC |
| tracrRNA | | | | | | GCCCTTGCAGCTGCTTCTCATGGGCAAGTGAAAACTTG |
| | | | | | | CTGGTGAGAGGGAAAAGTCATAATTTAAAGTCTCGTC |
| | | | | | | TTTCTAGTATGACATAGGTGCGCTCTCACGCAATATAG |
| | | | | | | GGTTCAGCTTTTATTTTATAGAAGTAGAGACTTTCCTC |
| | | | | | | TAGTGACAGTGCCGAAATGACCCCGTGCGAGGGGTAA |
| | | | | | | CTACCT |
| MG64 | 736 | MG64-124 | nucleotide | artificial | | TATTGAACAATAGCGTCGCAGTTCATGCTTTATGCTGA |
| effectors | | predicted | | sequence | | TGTGCTGCAACAAAAATGGGTCAGGTTGCCGCTGCAC |
| putative | | tracrRNA | | | | AGGCGGATTTGCTTTCCGGCCCTGGTTGCTACCCGTCC |
| tracrRNA | | | | | | TGGGAGATTCTTCCGGCGCTTAGGACATTACGGGTAGT |
| | | | | | | TACTGAAGCGCCTCCGAGCAAGGAGGAATCCTTCCTA |
| | | | | | | TTTTTTGGCAAACC |
| MG64 | 737 | MG64-107 | nucleotide | artificial | | GAGACAATAGCGCAGCAGTTCATGCTCATCGAGCCGC |
| effectors | | predicted | | sequence | | TGAACTGTGAAAAATGAGGGGTCAGTTTGACCGTTGT |
| putative | | tracrRNA | | | | GAGACGGTCGTGCTTTCCGCCCCTGAATAGCTGCCCGG |
| tracrRNA | | | | | | TCACTGACTGCCATCCTGGTTATTGTTCATTTTTGAGCA |
| | | | | | | ATGGATGGGGATGGGAAGAAATTACATTGAAAAGAGT |
| | | | | | | CTCTTCTCCAATGTAACGTAGGTGCGCACCCAGCAGAA |
| | | | | | | GTTGATCCTAGCCTTCAGCAATGAAGGTACAGCCTAGT |
| | | | | | | CTGTAGCGGCAGCTATTAATTGCCTCCGATCAAGGAG |
| | | | | | | GAGTCCTCC |
| MG64 | 738 | MG64-125 | nucleotide | artificial | | ATGTAAATAGCGCCGCAACGTATTCTGCTTGCAGCGTA |
| effectors | | predicted | | sequence | | CGTTCCGTGAAAAATGAGGGTTAGTTTGGCTGTCGGCA |
| putative | | tracrRNA | | | | GGCAGTCTTGCTTTCTGACCCTGGTAGCTGCTCACCCT |
| tracrRNA | | | | | | GATGCTGTCAGGAAAGAGACTTCGGTTTCTCAACTGA |
| | | | | | | GATTAAGTCGTAATTGAAGAGCTATTCTCCTTTAATTA |
| | | | | | | TGGCGTAGGTGCGCTCCCAGCAAAAAGGGCGCAGATA |
| | | | | | | TACTGCTGTAGTGGCTACCGAATCACCCCCAACCAAG |
| | | | | | | GGGGAACCCGCT |
| MG64 | 739 | MG64-127 | nucleotide | artificial | | TAACTAAAAGCGTCGCAATTCATGGCTTATTAATAAGT |
| effectors | | predicted | | sequence | | CCTCTGCATCGCCGAAAAATAGGGTTAGTTTGATTGTC |
| putative | | tracrRNA | | | | GGAAGATGATTTTTCTTTCTGACCCTGGTAAGTGCCCA |
| tracrRNA | | | | | | CTTCTGAAGCTGCTGTCTCTAGCCCTCGCTAATGTAGA |
| | | | | | | TAGGAAAGTGCCTTAATTTAGATCTCGTAACTCTATAT |
| | | | | | | TAACGGTCAGGTGCGCTCCCAGCAATAAGAAGTAAGT |
| | | | | | | CTTCAAGATGAGGAACAACTTGAGACTAATCCCTAAG |
| | | | | | | CGGGATACAGATTACTGGAGTGGTAGTTACTGAATCA |
| | | | | | | CCTCCTTCATCGGAGGAATCCTTC |

What is claimed is:

1. A system for transposing a cargo nucleotide sequence into a target nucleic acid site in a target nucleic acid comprising:
a) a Cas effector complex comprising a class 2, type II Cas effector, a small prokaryotic ribosomal protein subunit S15, and an engineered guide polynucleotide configured to hybridize to the target nucleic acid site;
b) a recombinase or transposase complex configured to bind the Cas effector complex; and
c) a double-stranded nucleic acid configured to interact with the recombinase or transposase complex and comprising the cargo nucleotide sequence.

2. The system of claim 1, wherein the Cas effector complex binds non-covalently to the recombinase or transposase complex.

3. The system of claim 1, wherein the Cas effector complex is covalently linked to the recombinase or transposase complex.

4. The system of claim 1, wherein the Cas effector complex is fused to the recombinase or transposase complex.

5. The system of any one of claims 1-4, wherein the cargo nucleotide sequence is flanked by a left-hand transposase recognition sequence and a right-hand transposase recognition sequence recognized by the recombinase or transposase complex.

6. The system of claim 5, wherein the left-hand recombinase sequence comprises a sequence having at least 80% identity to any one of SEQ ID NOs: 17-18.

7. The system of any one of claim 5, wherein the right-hand recombinase sequence comprises a sequence having at least 80% identity to SEQ ID NO: 19.

8. The system of claim 1-7, wherein the target nucleic acid comprises a PAM sequence compatible with the Cas effector complex.

9. The system of claim 8, wherein the PAM sequence is located about 50 to about 70 base pairs from the target nucleic acid site.

10. The system of claim 9, wherein the PAM sequence is located 3′ of the target nucleic acid site.

11. The system of claim 9, wherein the PAM sequence is located 5′ of the target nucleic acid site.

12. The system of any one of claims 1-11, wherein the class 2, type II Cas effector is not a Cas12k effector.

13. The system of any one of claims 1-11, wherein the class 2, type II Cas effector comprises a polypeptide comprising a sequence having at least 80% identity to SEQ ID NO: 1.

14. The system of any one of claims 1-11, wherein the class 2, type II Cas effector comprises a polypeptide comprising a sequence having at least 90% identity to SEQ ID NO: 1.

15. The system of any one of claims 1-11, wherein the class 2, type II Cas effector comprises a polypeptide comprising a sequence of SEQ ID NO: 1.

16. The system of any one of claims 1-15, wherein the recombinase or transposase complex comprises at least one polypeptide comprising a sequence having at least 80% identity to any one of SEQ ID NOs: 2-5.

17. The system of any one of claims 1-15, wherein the recombinase or transposase complex comprises at least one polypeptide comprising a sequence having at least 90% identity to any one of SEQ ID NOs: 2-5.

18. The system of any one of claims 1-17, wherein the recombinase or transposase complex comprises at least one polypeptide comprising a sequence of any one of SEQ ID NOs: 2-5.

19. The system of any one of claims 1-18, wherein the engineered guide polynucleotide comprises a sequence comprising at least about 46-80 consecutive nucleotides having at least 80% identity to SEQ ID NO: 12.

20. The system of any one of claims 1-18, wherein the engineered guide polynucleotide comprises a sequence having at least 80% sequence identity to SEQ ID NO: 11.

21. The system of any one of claims 1-20, wherein the small prokaryotic ribosomal protein subunit S15 comprises a sequence having at least 80% sequence identity to any one of any one of SEQ ID NOs: 494-659.

22. The system of any one of claims 1-21, wherein the class 2, type II Cas effector and the recombinase or transposase complex are encoded by polynucleotide sequences comprising fewer than about 10 kilobases.

23. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide polynucleotide configured to hybridize to the target nucleic acid site;
b) a Tn7 type transposase complex configured to bind the Cas effector complex and comprising a TnsA, TnsB, TnsC, and TniQ component; and
c) a double-stranded nucleic acid configured to interact with the Tn7 type transposase complex and comprising the cargo nucleotide sequence.

24. The system of claim 23, wherein the Cas effector complex binds non-covalently to the Tn7 type transposase complex.

25. The system of claim 23, wherein the Cas effector complex is covalently linked to the Tn7 type transposase complex.

26. The system of claim 23, wherein the Cas effector complex is fused to the Tn7 type transposase complex.

27. The system of any one of claims 23-26, wherein the cargo nucleotide sequence is flanked by a left-hand transposase recognition sequence and a right-hand transposase recognition sequence recognized by the recombinase or transposase complex.

28. The system of claim 27, wherein the left-hand recombinase sequence comprises a sequence having at least 80% identity to any one of SEQ ID NOs: 20.

29. The system of claim 27, wherein the right-hand recombinase sequence comprises a sequence having at least 80% identity to SEQ ID NO: 21.

30. The system of any one of claims 23-29, wherein the target nucleic acid comprises a PAM sequence compatible with the Cas effector complex.

31. The system of claim 30, wherein the PAM sequence is located about 50 to about 70 base pairs from the target nucleic acid site.

32. The system of claim 31, wherein the PAM sequence is located 3′ of the target nucleic acid site.

33. The system of claim 31, wherein the PAM sequence is located 5′ of the target nucleic acid site.

34. The system of any one of claims 23-33, wherein the class 2, type V Cas effector is not a Cas12k effector.

35. The system of any one of claims 23-34, wherein the TnsA component comprises a polypeptide comprising a sequence having at least 80% identity to SEQ ID NO: 7.

36. The system of any one of claims 23-25, wherein the Tn7 type transposase complex comprises at least one polypeptide comprising a sequence having at least 80% identity to any one of SEQ ID NOs: 8-10.

37. The system of any one of claims 23-36, wherein the engineered guide polynucleotide comprises a sequence comprising at least about 46-80 consecutive nucleotides having at least 80% identity to any one of SEQ ID NOs: 13-16.

38. The system of any one of claims 23-37, wherein the small prokaryotic ribosomal protein subunit S15 comprises a sequence having at least 80% sequence identity to any one of any one of SEQ ID NOs: 494-659.

39. The system of any one of claims 23-38, wherein the class 2, type II Cas effector and the recombinase or transposase complex are encoded by polynucleotide sequences comprising fewer than about 10 kilobases.

40. A system for transposing a cargo nucleotide sequence into a target nucleic acid site in a target nucleic acid comprising:
a) a Cas effector complex comprising a class 1, type I-F Cas effector, a small prokaryotic ribosomal protein subunit S15, and an engineered guide polynucleotide configured to hybridize to the target nucleic acid site;
b) a Tn7 type transposase complex configured to bind the Cas effector complex and comprising a TnsA, TnsB, TnsC, and TniQ component; and
c) a double-stranded nucleic acid configured to interact with the Tn7 type transposase complex and comprising a cargo nucleotide sequence.

41. The system of claim 40, wherein the Cas effector complex binds non-covalently to the Tn7 type transposase complex.

42. The system of claim 40, wherein the Cas effector complex is covalently linked to the Tn7 type transposase complex.

43. The system of claim 40, wherein the Cas effector complex is fused to the Tn7 type transposase complex.

44. The system of any one of claims 40-43, wherein the cargo nucleotide sequence is flanked by a left-hand transposase recognition sequence and a right-hand transposase recognition sequence recognized by the recombinase or transposase complex.

45. The system of claim 44, wherein the left-hand recombinase sequence comprises a sequence having at least 80% identity to any one of SEQ ID NOs: 136 and 138.

46. The system of any one of claim 44, wherein the right-hand recombinase sequence comprises a sequence having at least 80% identity to SEQ ID NO: 137 and 139.

47. The system of claim 40-46, wherein the target nucleic acid comprises a PAM sequence compatible with the Cas effector complex.

48. The system of claim 47, wherein the PAM sequence is located about 50 to about 70 base pairs from the target nucleic acid site.

49. The system of claim 48, wherein the PAM sequence is located 3′ of the target nucleic acid site.

50. The system of claim 48, wherein the PAM sequence is located 5′ of the target nucleic acid site.

51. The system of any one of claims 40-50, wherein the class 1, type I-F Cas effector comprises a polypeptide comprising a sequence having at least 80% identity to any one of SEQ ID NOs: 41-43 and 48-50.

52. The system of any one of claims 40-50, wherein the class 1, type I-F Cas effector comprises a polypeptide comprising a sequence having at least 90% identity to any one of SEQ ID NOs: 41-43 and 48-50.

53. The system of any one of claims 40-50, wherein the class 1, type I-F Cas effector comprises a polypeptide comprising a sequence of any one of SEQ ID NOs: 41-43 and 48-50.

54. The system of any one of claims 40-53, wherein the Tn7 type transposase complex comprises at least one polypeptide comprising a sequence having at least 80% identity to any one of SEQ ID NOs: 44-47 and 51-54.

55. The system of any one of claims 40-53, wherein the Tn7 type transposase complex comprises at least one polypeptide comprising a sequence having at least 90% identity to any one of SEQ ID NOs: 44-47 and 51-54.

56. The system of any one of claims 40-53, wherein the Tn7 type transposase complex comprises at least one polypeptide comprising a sequence of any one of SEQ ID NOs: 44-47 and 51-54.

57. The system of any one of claims 40-56, wherein the small prokaryotic ribosomal protein subunit S15 comprises a sequence having at least 80% sequence identity to any one of any one of SEQ ID NOs: 494-659.

58. The system of any one of claims 40-57, wherein the class 2, type II Cas effector and the recombinase or transposase complex are encoded by polynucleotide sequences comprising fewer than about 10 kilobases.

59. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to any one of SEQ ID NOs: 22, 26, 30, 34, 55-89, 104, 147, 264-304, and 660-689; and
ii) an engineered guide polynucleotide comprising a sequence having at least 80% sequence identity to any one of SEQ ID NOs: 90-93, 111-114, 117, 151, 156-181, 201-204, 209-234, 255-258, 262, 263, 348, 350-353, 417-460, 491-492, and 715-739;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 23-25, 27-29, 31-33, 35-37, 101-103, 105-107, 148-150, 305-343, and 345-347; and
c) a double-stranded nucleic acid configured to interact with the Tn7 type transposase complex and comprising in 5′ to 3′ order:
i) a left-hand recombinase sequence comprising a sequence having at least 80% sequence identity to any one of SEQ ID NOs: 123, 125, 127, 129, 131, 133, 153, 354-358, 461, 463, 465, and 467;
ii) the cargo nucleotide sequence; and
iii) a right-hand recombinase sequence comprising a sequence having at least 80% identity to any one of SEQ ID NOs: 124, 126, 128, 130, 132, 154, 155, 359-363, 462, 464, 466, and 468.

60. A system for transposing a cargo nucleotide sequence into a target nucleic acid site comprising:
a) a Cas effector complex configured to hybridize to the target nucleic acid site and comprising:
i) a class 2, type V Cas effector comprising a polypeptide having a sequence having at least 80% sequence identity to SEQ ID NO: 22; and
ii) an engineered guide polynucleotide comprising a sequence having at least 80% sequence identity to any one of SEQ ID NOs: 90, 112, and 202;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 23-25; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 125;
ii) the cargo nucleotide sequence; and
iii) a right-hand recombinase sequence comprising a sequence having at least 80% identity to any one of SEQ ID NOs: 126 and 155.

61. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to SEQ ID NO: 26; and
ii) an engineered guide polynucleotide comprising a sequence having at least 80% sequence identity to any one of SEQ ID NOs: 91, 113, 156, 203, and 209;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 27-29; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 127;
ii) the cargo nucleotide sequence; and
iii) a right-hand recombinase sequence comprising a sequence having at least 80% identity to SEQ ID NO: 128.

62. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to SEQ ID NO: 60; and
ii) an engineered guide polynucleotide comprising a sequence having at least 80% sequence identity to any one of SEQ ID NOs: 117, 119, 161, and 214;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 101-103; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 131;
ii) the cargo nucleotide sequence; and
iii) a right-hand recombinase sequence comprising a sequence having at least 80% identity to SEQ ID NO: 132.

63. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to SEQ ID NO: 147; and
ii) an engineered guide polynucleotide comprising a sequence having at least 80% sequence identity to any one of SEQ ID NOs: 151, 181, and 234;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 148-150; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 153;
ii) the cargo nucleotide sequence; and
iii) a right-hand recombinase sequence comprising a sequence having at least 80% identity to SEQ ID NO: 154.

64. A system for transposing a cargo nucleotide sequence into a target nucleic acid site comprising:
a) a Cas effector complex configured to hybridize to the target nucleic acid site in a target nucleic acid and comprising:
i) a class 2, type V Cas effector comprising a polypeptide having a sequence having at least 80% sequence identity to SEQ ID NO: 34; and
ii) an engineered guide polynucleotide comprising a sequence having at least 80% sequence identity to any one of SEQ ID NOs: 93, 114, 157, 204, and 210;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 148-150; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 129;
ii) the cargo nucleotide sequence; and
iii) a right-hand recombinase sequence comprising a sequence having at least 80% identity to SEQ ID NO: 130.

65. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to SEQ ID NO: 30; and
ii) an engineered guide polynucleotide comprising a sequence having at least 80% sequence identity to any one of SEQ ID NOs: 92, 111, and 201;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 31-33; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 123;
ii) the cargo nucleotide sequence; and
iii) a right-hand recombinase sequence comprising a sequence having at least 80% identity to SEQ ID NO: 124.

66. A system for transposing a cargo nucleotide sequence into a target nucleic acid site in a target nucleic acid comprising:
a) a Cas effector complex configured to hybridize to the target nucleic acid site and comprising:
i) a class 2, type V Cas effector comprising a polypeptide having a sequence having at least 80% sequence identity to SEQ ID NO: 38; and
ii) an engineered guide polynucleotide comprising a sequence having at least 80% sequence identity to any one of SEQ ID NOs: 98, 115-116, 182, 205-206, 235, and 493;
b) a Tn7 type transposase complex configured to bind the Cas effector complex and comprising TnsB, TnsC, and TniQ components, the TnsB, TnsC, or TniQ component comprising a sequence having at least 80% identity to any one of SEQ ID NOs: 39 and 40; and
c) a double-stranded nucleic acid configured to interact with the Tn7 type transposase complex and comprising in 5′ to 3′ order:
i) a left-hand recombinase sequence comprising a sequence having at least 80% sequence identity to SEQ ID NO: 134;
ii) the cargo nucleotide sequence; and
iii) a right-hand recombinase sequence comprising a sequence having at least 80% identity to SEQ ID NO: 135.

67. The system of any one of claims 59-66, wherein the class 2, type V Cas effector is a Cas12k effector.

68. The system of any one of claims 59-67, wherein the target nucleic acid comprises a PAM sequence compatible with the Cas effector complex.

69. The system of claim 68, wherein the PAM sequence is located 5′ of the target nucleic acid site.

70. The system of any one of claims 68-69, wherein the PAM sequence comprises 5′-nGTn-3′ or 5′-nGTt-3′.

71. The system of any one of claims 59-70, wherein the Cas effector complex further comprises a small prokaryotic ribosomal protein subunit S15.

72. The system of claim 71, wherein the small prokaryotic ribosomal protein subunit S15 comprises a sequence having at least 80% sequence identity to any one of SEQ ID NOs: 494-659.

73. The system of claim 71, wherein the class 2, type V Cas effector and the Tn7 type transposase complex are encoded by polynucleotide sequences comprising fewer than about 10 kilobases.

74. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide polynucleotide configured to hybridize to the target nucleic acid site;
b) a Tn7 type transposase complex configured to bind the Cas effector complex and comprising TnsB and TnsC components but not a TnsA and/or TniQ component; and
c) a double-stranded nucleic acid configured to interact with the Tn7 type transposase complex and comprising the cargo nucleotide sequence.

75. The system of claim 74, wherein the Cas effector complex binds non-covalently to the Tn7 type transposase complex.

76. The system of claim 74, wherein the Cas effector complex is covalently linked to the Tn7 type transposase complex.

77. The system of claim 74, wherein the Cas effector complex is fused to the Tn7 type transposase complex.

78. The system of any one of claims 74-77, wherein the cargo nucleotide sequence is flanked by a left-hand transposase recognition sequence and a right-hand transposase recognition sequence recognized by the recombinase or transposase complex.

79. The system of claim 78, wherein the left-hand recombinase sequence comprises a sequence having at least 80% identity to any one of SEQ ID NOs: 134.

80. The system of claim 78, wherein the right-hand recombinase sequence comprises a sequence having at least 80% identity to SEQ ID NO: 135.

81. The system of any one of claims 74-80, wherein the target nucleic acid comprises a PAM sequence compatible with the Cas effector complex.

82. The system of claim 81, wherein the PAM sequence is located about 50 to about 70 base pairs from the target nucleic acid site.

83. The system of claim 82, wherein the PAM sequence is located 3′ of the target nucleic acid site.

84. The system of claim 82, wherein the PAM sequence is located 5′ of the target nucleic acid site.

85. The system of any one of claims 74-84, wherein the class 2, type V Cas effector is a Cas12k effector.

86. The system of any one of claims 74-85, wherein the class 2, type V Cas effector comprises a polypeptide comprising a sequence having at least 80% identity to any one of SEQ ID NOs: 38 and 108.

87. The system of any one of claims 74-85, wherein the class 2, type V Cas effector comprises a polypeptide comprising a sequence having at least 90% identity to any one of SEQ ID NOs: 38 and 108.

88. The system of any one of claims 74-85, wherein the class 2, type V Cas effector comprises a polypeptide comprising a sequence of any one of SEQ ID NOs: 38 and 108.

89. The system of any one of claims 74-88, wherein the TnsB subunit comprises a polypeptide comprising a sequence having at least 80% identity to SEQ ID NOs: 40 or 109.

90. The system of any one of claims 74-89, wherein the TnsC subunit comprises a polypeptide comprising a sequence having at least 80% identity to SEQ ID NOs: 39 or 110.

91. The system of any one of claims 74-90, wherein the Tn7 type transposase complex comprises at least one polypeptide comprising a sequence having at least 80% identity to any one of SEQ ID NOs: 39-40, 109-110, and 344.

92. The system of any one of claims 74-91, wherein the engineered guide polynucleotide comprises a sequence having at least 80% identity to any one of SEQ ID NOs: 115, 116, 205, 206, 261, 235, 260, and 236.

93. The system of any one of claims 74-91, wherein the engineered guide polynucleotide comprises a sequence comprising at least about 46-80 consecutive nucleotides having at least 80% identity to any one of SEQ ID NOs: 118, 182, 183, 235, and 236.

94. The system of any one of claims 74-93, wherein the small prokaryotic ribosomal protein subunit S15 comprises a sequence having at least 80% sequence identity to any one of SEQ ID NOs: 494-659.

95. The system of any one of claims 74-94, wherein the class 2, type II Cas effector and the recombinase or transposase complex are encoded by polynucleotide sequences comprising fewer than about 10 kilobases.

96. A system for transposing a cargo nucleotide sequence into a target nucleic acid site in a target nucleic acid comprising:
a) a Cas effector complex comprising a class 2, type II Cas effector, a small prokaryotic ribosomal protein subunit S15, and an engineered guide polynucleotide, the engineered guide polynucleotide capable of hybridizing to the target nucleic acid;
b) a recombinase or transposase complex operably linked to the Cas effector complex; and
c) a double-stranded nucleic acid comprising in 5′ to 3′ order:
i) a left-hand recombinase recognition sequence;
ii) the cargo nucleotide sequence; and
iii) a right-hand recombinase recognition sequence, the left-hand recombinase recognition sequence and the right-hand recombinase recognition sequence capable of being recognized by the recombinase or transposase complex.

97. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide polynucleotide, the engineered guide polynucleotide capable of hybridizing to the target nucleic acid;
b) a Tn7 type transposase complex operably linked to the Cas effector complex and comprising a TnsA, TnsB, TnsC, and TniQ component; and
c) a double-stranded nucleic acid comprising in 5′ to 3′ order:
i) a left-hand recombinase recognition sequence;
ii) the cargo nucleotide sequence; and
iii) a right-hand recombinase recognition sequence, the left-hand recombinase recognition sequence and the right-hand recombinase recognition sequence capable of being recognized by the Tn7 type transposase complex.

98. A system for transposing a cargo nucleotide sequence into a target nucleic acid site in a target nucleic acid comprising:
a) a Cas effector complex comprising a class 1, type I-F Cas effector, a small prokaryotic ribosomal protein subunit $15, and an engineered guide polynucleotide, the engineered guide polynucleotide capable of hybridizing to the target nucleic acid;
b) a Tn7 type transposase complex operably linked to the Cas effector complex and comprising a TnsA, TnsB, TnsC, and TniQ component; and
c) a double-stranded nucleic acid comprising in 5′ to 3′ order:
i) a left-hand recombinase recognition sequence;
ii) the cargo nucleotide sequence; and
iii) a right-hand recombinase recognition sequence, the left-hand recombinase recognition sequence and the right-hand recombinase recognition sequence capable of being recognized by the Tn7 type transposase complex.

99. An engineered nuclease system comprising:
an endonuclease comprising a RuvC domain and an HNH domain, wherein the endonuclease is derived from an uncultivated microorganism, wherein the endonuclease is a Class 2, type II endonuclease comprising a sequence having at least 80% identity to SEQ ID NO: 1; and
an engineered guide polynucleotide, wherein the engineered guide RNA is configured to form a complex with the endonuclease and the engineered guide RNA comprises a spacer sequence configured to hybridize into a target nucleic acid sequence.

100. The engineered nuclease system of claim 99, wherein the engineered guide polynucleotide comprises at least 60-80 consecutive nucleotides having at least 80% identity to SEQ ID NO: 12.

101. The engineered nuclease system of claim 99, wherein the engineered guide polynucleotide comprises a sequence having at least 80% identity to SEQ ID NO: 11.

102. An engineered nuclease system comprising:
an endonuclease comprising a RuvC domain, wherein the endonuclease is derived from an uncultivated microorganism, and wherein the endonuclease is a Class 2, type V endonuclease having at least 80% identity to SEQ ID NO: 6; and
an engineered guide RNA, wherein the engineered guide RNA is configured to form a complex with the endonuclease and the engineered guide RNA comprises a spacer sequence configured to hybridize into a target nucleic acid sequence.

103. The engineered nuclease system of claim 102, wherein the engineered guide polynucleotide comprises a sequence comprising at least about 46-80 consecutive nucleotides having at least 80% identity to any one of SEQ ID NOs: 13-16.

104. An engineered nuclease system comprising:
an endonuclease comprising a RuvC domain, wherein the endonuclease is derived from an uncultivated microorganism, and wherein the endonuclease is a Class 2, type V-K endonuclease having at least 80% identity to any one of SEQ ID NOs: 22, 26, 30, 34, 55-89, 104, 147, 264-304, and 660-689; and
an engineered guide RNA, wherein the engineered guide RNA is configured to form a complex with the endonuclease and the engineered guide RNA comprises a spacer sequence configured to hybridize into a target nucleic acid sequence.

105. The engineered nuclease system of claim 104, wherein the engineered guide polynucleotide comprises a sequence comprising at least about 46-80 consecutive nucleotides having at least 80% identity to any one of SEQ ID NOs: 90-93, 117, 151, 156-181, 209-234, 417-460, and 715-739.

106. The engineered nuclease system of claim 104 or 105, wherein the engineered guide polynucleotide comprises a sequence having at least 80% sequence identity to any one of SEQ ID NOs: 111-114, 201-206, 209, 210, 255-258, 262, 263, 348, 350-353, and 473-492.

107. An engineered nuclease system comprising:
an endonuclease comprising a RuvC domain, wherein the endonuclease is derived from an uncultivated microorganism, and wherein the endonuclease is a Class 2, type V-K endonuclease having at least 80% identity to SEQ ID NO: 38 or SEQ ID NO: 108; and
an engineered guide RNA, wherein the engineered guide RNA is configured to form a complex with the endonuclease and the engineered guide RNA comprises a spacer sequence configured to hybridize into a target nucleic acid sequence.

108. The engineered nuclease system of claim 107, wherein the engineered guide polynucleotide comprises a sequence comprising at least about 46-80 consecutive nucleotides having at least 80% identity to any one of SEQ ID NOs: 118, 182, 183, 235, and 236.

109. The engineered nuclease system of claim 107, wherein the engineered guide polynucleotide comprises a sequence having at least 80% identity to any one of SEQ ID NOs: 111-114, 115, 116, 201-206, 209, 210, 235, 236, 255-258, 260-263, 348, and 350-353.

110. An engineered nuclease system comprising:
a Class 1, type I-F Cas endonuclease comprising at least one Cas6, Cas7, or Cas8 polypeptide comprising a sequence having at least 80% identity to any one of SEQ ID NOs: 41-43 and 48-50; and
an engineered guide RNA, wherein the engineered guide RNA is configured to form a complex with the endonuclease and the engineered guide RNA comprises a spacer sequence configured to hybridize into a target nucleic acid sequence.

111. The engineered nuclease system of claim 110, wherein the engineered guide polynucleotide comprises a sequence having at least 80% identity to any one of SEQ ID NOs: 121, 122, 207, and 208.

112. A method for transposing a cargo nucleotide sequence into a target nucleic acid site comprising introducing the system of any one of claims 1-111 to a cell.

113. A cell comprising the system of any one of claims 1-111.

114. The cell of claim 113, wherein the cell is a eukaryotic cell.

115. The cell of claim 113, wherein the cell is a mammalian cell.

116. The cell of claim 113, wherein the cell is an immortalized cell.

117. The cell of claim 113, wherein the cell is an insect cell.

118. The cell of claim 113, wherein the cell is a yeast cell.

119. The cell of claim 113, wherein the cell is a plant cell.

120. The cell of claim 113, wherein the cell is a fungal cell.

121. The cell of claim 113, wherein the cell is a prokaryotic cell.

122. The cell of claim 113, wherein the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof.

123. The cell of claim 113, wherein the cell is an engineered cell.

124. The cell of claim 113, wherein the cell is a stable cell.
